(19) United States
(12) Patent Application Publication
(10) Pub. No.: US 2024/0010636 A1
(43) Pub. Date: Jan. 11, 2024

(54) HETEROCYCLIC COMPOUND, ORGANIC LIGHT-EMITTING DEVICE INCLUDING THE SAME, AND ELECTRONIC APPARATUS INCLUDING THE ORGANIC LIGHT-EMITTING DEVICE

(71) Applicant: Samsung Electronics Co., Ltd., Suwon-si (KR)

(72) Inventors: Yeon Sook Chung, Suwon-si (KR); Eunjeong Choi, Suwon-si (KR); Eunhye AN, Osan-si (KR); Heechoon Ahn, Seoul (KR); Giwook Kang, Yongin-si (KR); Eunsuk Kwon, Suwon-si (KR); Yeseul Lee, Hwaseong-si (KR); Dongjin Jang, Suwon-si (KR); Seowon Cho, Hwaseong-si (KR); Yongsik Jung, Suwon-si (KR); Hwang Suk Kim, Suwon-si, Gyeonggi-do (KR); Hyeonho Choi, Suwon-si (KR); Byoungki Choi, Suwon-si (KR)

(21) Appl. No.: 18/312,185

(22) Filed: May 4, 2023

(30) Foreign Application Priority Data
May 4, 2022 (KR) 10-2022-0055745
May 3, 2023 (KR) 10-2023-0057772

(51) Int. Cl. C07D 403/14; H10K 85/60; H10K 50/12; C09K 11/06; H10K 50/15; H10K 50/16

(52) U.S. Cl. CPC C07D 403/14; H10K 85/6572; H10K 50/12; C07B 2200/05; H10K 50/15; H10K 50/16; C09K 2211/1059; C09K 11/06

(57) ABSTRACT

A heterocyclic compound represented by Formula 1:

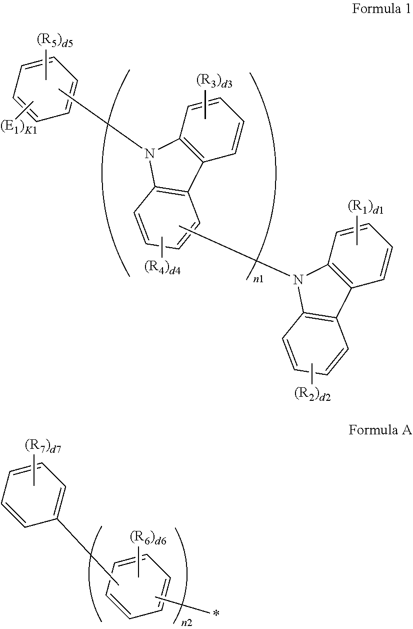

wherein E₁ is a group represented by Formula A; k1 is an integer from 1 to 5; n1 is 2 or 3; n2 is an integer from 0 to 3; d1, d2, d3, d5, and d6 are each independently an integer from 0 to 4; d4 is an integer from 0 to 3; d7 is an integer from 0 to 5; wherein the heterocyclic compound represented by Formula 1 comprises at least one deuterium; * indicates a binding site to an adjacent atom; and the remaining substituents are as defined herein.

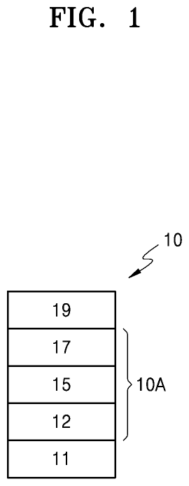

### CROSS-REFERENCE TO RELATED APPLICATION

This application claims priority to and the benefit of Korean Patent Application Nos. 10-2022-0055745, filed on May 4, 2022, and 10-2023-0057772, filed on May 3, 2023, in the Korean Intellectual Property Office, and all the benefits accruing therefrom under 35 U.S.C. § 119, the entire contents of which are incorporated by reference herein.

### BACKGROUND

### 1. Field

The present subject matter relates to a heterocyclic compound, an organic light-emitting device including the same, and an electronic apparatus including the organic light-emitting device.

### 2. Description of the Related Art

Organic light-emitting devices (OLEDs) are self-emissive devices, which have improved characteristics in terms of viewing angles, response time, brightness, driving voltage, and response speed. In addition, OLEDs produce full-color images.

In an example, an organic light-emitting device includes an anode, a cathode, and an organic layer that is arranged between the anode and the cathode and includes an emission layer. A hole transport region may be provided between the anode and the emission layer, and an electron transport region may be provided between the emission layer and the cathode. Holes provided from the anode may move toward the emission layer through the hole transport region, and electrons provided from the cathode may move toward the emission layer through the electron transport region. The holes and the electrons may recombine in the emission layer to produce excitons. The excitons may transition from an excited state to a ground state, thereby generating light.

### SUMMARY

Provided are a heterocyclic compound, an organic light-emitting device including the same, and an electronic apparatus including the organic light-emitting device.

Additional aspects will be set forth in part in the detailed description that follows and, in part, will be apparent from the detailed description, or may be learned by practice of the presented exemplary embodiments provided herein.

According to an aspect, there is provided a heterocyclic compound represented by Formula 1:

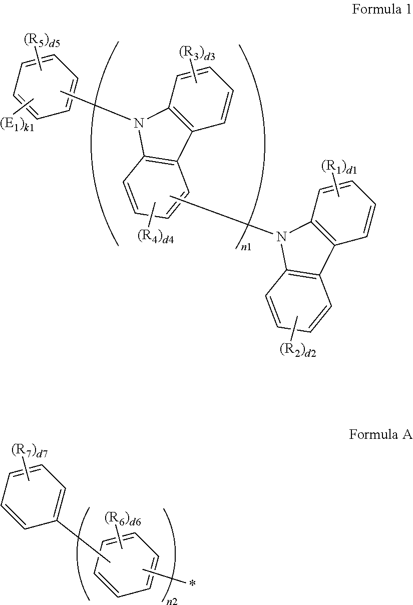

wherein, in Formulae 1 and A, E₁ is a group represented by Formula A,
k1 is an integer from 1 to 5,
n1 is 2 or 3,
n2 is an integer from 0 to 3,
R₁ to R₆ are each independently hydrogen, deuterium, —F, —Cl, —Br, —I, —SF₅, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₁-C₆₀ alkylthio group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₇-C₆₀ alkyl aryl group, a substituted or unsubstituted C₇-C₆₀ aryl alkyl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted C₂-C₆₀ alkyl heteroaryl group, a substituted or unsubstituted C₂-C₆₀ heteroaryl alkyl group, a substituted or unsubstituted C₁-C₆₀ heteroaryloxy group, a substituted or unsubstituted C₁-C₆₀ heteroarylthio group, a substituted or unsubstituted monovalent non-aromatic condensed polycyclic group, a substituted or unsubstituted monovalent non-aromatic condensed heteropolycyclic group, —N(Q₁)(Q₂), —Si(Q₃)(Q₄)(Q₅), —Ge(Q₃)(Q₄)(Q₅), —B(Q₆)(Q₇), —P(═O)(Q₈)(Q₉), or —P(Q₈)(Q₉),
R₇ is hydrogen, deuterium, —F, —Cl, —Br, —I, —SF₅, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₁-C₆₀ alkylthio group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryloxy group, a substituted or unsubstituted C₁-C₆₀ heteroaryloxy group, a substituted or unsubstituted C₁-C₆₀ heteroarylthio group, a substituted or unsubstituted C₆-C₆₀ arylthio group, —N(Q₁)(Q₂), —Si(Q₃)(Q₄)(Q₅), —Ge(Q₃)(Q₄)(Q₅), —B(Q₆)(Q₇), —P(═O)(Q₈)(Q₉), or —P(Q₈)(Q₉),
d1, d2, d3, d5, and d6 are each independently an integer from 0 to 4,
d4 is an integer from 0 to 3,
d7 is an integer from 0 to 5,
the heterocyclic compound represented by Formula 1 includes at least one deuterium,
at least one substituent of the substituted C₁-C₆₀ alkyl group, the substituted C₂-C₆₀ alkenyl group, the substituted C₂-C₆₀ alkynyl group, the substituted C₁-C₆₀ alkoxy group, the substituted C₁-C₆₀ alkylthio group, the substituted C₃-C₁₀ cycloalkyl group, the substituted C₁-C₁₀ heterocycloalkyl group, the substituted C₃-C₁₀ cycloalkenyl group, the substituted C₁-C₁₀ heterocycloalkenyl group, the substituted C₆-C₆₀ aryl group, the substituted C₇-C₆₀ alkyl aryl group, the substituted C₇-C₆₀ aryl alkyl group, the substituted C₆-C₆₀ aryloxy group, the substituted C₆-C₆₀ arylthio group, the substituted C₁-C₆₀ heteroaryl group, the substituted C₂-C₆₀ alkyl heteroaryl group, the substituted C₂-C₆₀ heteroaryl alkyl group, the substituted C₁-C₆₀ heteroaryloxy group, the substituted C₁-C₆₀ heteroarylthio group, the substituted monovalent non-aromatic condensed polycyclic group, and the substituted monovalent non-aromatic condensed heteropolycyclic group is:
deuterium, —F, —Cl, —Br, —I, —SF₅, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, or a C₁-C₆₀ alkylthio group,
a C₁-C₆₀ alkyl group, a C₂-C₆₀ alkenyl group, a C₂-C₆₀ alkynyl group, a C₁-C₆₀ alkoxy group, or a C₁-C₆₀ alkylthio group, each substituted with at least one of deuterium, —F, —Cl, —Br, —I, —SF₅, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₇-C₆₀ alkyl aryl group, a C₆-C₆₀ aryloxy group, a C₆-C₆₀ arylthio group, a C₁-C₆₀ heteroaryl group, a C₂-C₆₀ alkyl heteroaryl group, a C₁-C₆₀ heteroaryloxy group, a C₁-C₆₀ heteroarylthio group, a monovalent non-aromatic condensed polycyclic group, a monovalent non-aromatic condensed heteropolycyclic group, —N(Q₁₁)(Q₁₂), —Si(Q₁₃)(Q₁₄)(Q₁₅), —Ge(Q₁₃)(Q₁₄)(Q₁₅), —B(Q₁₆)(Q₁₇), —P(═O)(Q₁₈)(Q₁₉), —P(Q₁₈)(Q₁₉), or a combination thereof;
a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₇-C₆₀ alkyl aryl group, a C₆-C₆₀ aryloxy group, a C₆-C₆₀ arylthio group, a C₁-C₆₀ heteroaryl group, a C₂-C₆₀ alkyl heteroaryl group, a C₁-C₆₀ heteroaryloxy group, a C₁-C₆₀ heteroarylthio group, a monovalent non-aromatic condensed polycyclic group, or a monovalent non-aromatic condensed heteropolycyclic group, each unsubstituted or substituted with at least one of deuterium, —F, —Cl, —Br, —I, —SF₅, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₁-C₆₀ alkylthio group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₇-C₆₀ alkyl aryl group, a C₆-C₆₀ aryloxy group, a C₆-C₆₀ arylthio group, a C₁-C₆₀ heteroaryl group, a C₂-C₆₀ alkyl heteroaryl group, a C₁-C₆₀ heteroaryloxy group, a C₁-C₆₀ heteroarylthio group, a monovalent non-aromatic condensed polycyclic group, a monovalent non-aromatic condensed heteropolycyclic group, —N(Q₂₁)(Q₂₂), —Si(Q₂₃)(Q₂₄)(Q₂₅), —Ge(Q₂₃)(Q₂₄)(Q₂₅), —B(Q₂₆)(Q₂₇), —P(═O)(Q₂₈)(Q₂₉), —P(Q₂₈)(Q₂₉) or a combination thereof;
—N(Q₃₁)(Q₃₂), —Si(Q₃₃)(Q₃₄)(Q₃₅), —Ge(Q₃₃)(Q₃₄)(Q₃₅), —B(Q₃₆)(Q₃₇), —P(═O)(Q₃₈)(Q₃₉), or —P(Q₃₈)(Q₃₉); or
a combination thereof, and
Q₁ to Q₉, Q₁₁ to Q₁₉, Q₂₁ to Q₂₉, and Q₃₁ to Q₃₉ are each independently hydrogen, deuterium, —F, —Cl, —Br, —I, —SF₅, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₁-C₆₀ alkylthio group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₇-C₆₀ alkyl aryl group, a substituted or unsubstituted C₇-C₆₀ aryl alkyl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted C₂-C₆₀ alkyl heteroaryl group, a substituted or unsubstituted C₂-C₆₀ heteroaryl alkyl group, a substituted or unsubstituted C₁-C₆₀ heteroaryloxy group, a substituted or unsubstituted C₁-C₆₀ heteroarylthio group, a substituted or unsubstituted monovalent non-aromatic condensed polycyclic group, or a substituted or unsubstituted monovalent non-aromatic condensed heteropolycyclic group.

According to another aspect, an organic light-emitting device includes a first electrode, a second electrode, and an organic layer arranged between the first electrode and the second electrode, wherein the organic layer includes an emission layer, and wherein the organic layer further includes at least one of the heterocyclic compounds represented by Formula 1.

According to another aspect, an electronic apparatus includes the organic light-emitting device.

### BRIEF DESCRIPTION OF THE DRAWING

The above and other aspects, features, and advantages of certain exemplary embodiments will be more apparent from the following detailed description taken in conjunction with the FIGURE, which is a schematic cross-sectional view of an organic light-emitting device 10 according to one or more embodiments.

### DETAILED DESCRIPTION

Reference will now be made in further detail to exemplary embodiments, examples of which are illustrated in the accompanying drawing, wherein like reference numerals refer to like elements throughout. In this regard, the present exemplary embodiments may have different forms and should not be construed as being limited to the detailed descriptions set forth herein. Accordingly, the exemplary embodiments are merely described in further detail below, and by referring to the FIGURE, to explain certain aspects. As used herein, the term “and/or” includes any and all combinations of one or more of the associated listed items. Expressions such as “at least one of,” when preceding a list of elements, modify the entire list of elements and do not modify the individual elements of the list.

The terminology used herein is for the purpose of describing one or more exemplary embodiments only and is not intended to be limiting. As used herein, the singular forms “a,” “an,” and “the” are intended to include the plural forms as well, unless the context clearly indicates otherwise. The term “or” means “and/or.” It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

It will be understood that, although the terms first, second, third etc. may be used herein to describe various elements, components, regions, layers, and/or sections, these elements, components, regions, layers, and/or sections should not be limited by these terms. These terms are only used to distinguish one element, component, region, layer, or section from another element, component, region, layer, or section. Thus, a first element, component, region, layer, or section discussed below could be termed a second element, component, region, layer, or section without departing from the teachings of the present embodiments.

Exemplary embodiments are described herein with reference to cross section illustrations that are schematic illustrations of idealized embodiments. As such, variations from the shapes of the illustrations as a result, for example, of manufacturing techniques and/or tolerances, are to be expected. Thus, embodiments described herein should not be construed as limited to the particular shapes of regions as illustrated herein but are to include deviations in shapes that result, for example, from manufacturing. For example, a region illustrated or described as flat may, typically, have rough and/or nonlinear features. Moreover, sharp angles that are illustrated may be rounded. Thus, the regions illustrated in the figures are schematic in nature and their shapes are not intended to illustrate the precise shape of a region and are not intended to limit the scope of the present claims.

It will be understood that when an element is referred to as being “on” another element, it can be directly in contact with the other element or intervening elements may be present therebetween. In contrast, when an element is referred to as being “directly on” another element, there are no intervening elements present.

Unless otherwise defined, all terms (including technical and scientific terms) used herein have the same meaning as commonly understood by one of ordinary skill in the art to which this general inventive concept belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

“About” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within ±30%, 20%, 10%, 5% of the stated value.

As used herein, when a molecular fragment is described as being a substituent or otherwise attached to another moiety, its name may be written as if it were a fragment (for example, phenyl, naphthyl, or the like) or as if it were the whole molecule (for example, benzene, naphthalene, or the like). It is to be understood that the nomenclature may be used interchangeably herein.

As used herein, an “energy level” (e.g., a highest occupied molecular orbital (HOMO) energy level or a triplet (Ti) energy level) is expressed as an absolute value from a vacuum level. In addition, when the energy level is referred to as being “deep,” “high,” or “large,” the energy level has a large absolute value based on “0 electron Volts (eV)” of the vacuum level, and when the energy level is referred to as being “shallow,” “low,” or “small,” the energy level has a small absolute value based on “0 eV” of the vacuum level.

A heterocyclic compound according to an aspect is represented by Formula 1:

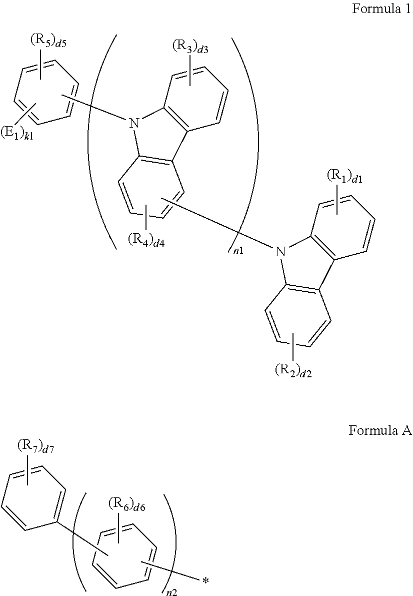

wherein, in Formulae 1,
E₁ is a group represented by Formula A, and
k1 is an integer from 1 to 5.

In one or more embodiments, k1 may be an integer from 1 to 3.

In Formulae 1 and A,
n1 is an integer from 1 to 3.

In one or more embodiments, n1 may be 2 or 3.

n2 is an integer from 0 to 3.

In one or more embodiments, n2 may be 0 or 1.

In Formulae 1 and A, R₁ to R₆ are each independently hydrogen, deuterium, —F, —Cl, —Br, —I, —SF₅, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₁-C₆₀ alkylthio group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₇-C₆₀ alkyl aryl group, a substituted or unsubstituted C₇-C₆₀ aryl alkyl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted C₂-C₆₀ alkyl heteroaryl group, a substituted or unsubstituted C₂-C₆₀ heteroaryl alkyl group, a substituted or unsubstituted C₁-C₆ heteroaryloxy group, a substituted or unsubstituted C₁-C₆₀ heteroarylthio group, a substituted or unsubstituted monovalent non-aromatic condensed polycyclic group, a substituted or unsubstituted monovalent non-aromatic condensed heteropolycyclic group, —N(Q₁)(Q₂), —Si(Q₃)(Q₄)(Q₅), —Ge(Q₃)(Q₄)(Q₅), —B(Q₆)(Q₇), —P(═O)(Q₈)(Q₉), or —P(Q₈)(Q₉).

In Formula A, R₇ is hydrogen, deuterium, —F, —Cl, —Br, —I, —SF₅, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₁-C₆₀ alkylthio group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryloxy group, a substituted or unsubstituted C₆-C₆₀ arylthio group, a substituted or unsubstituted C₁-C₆₀ heteroaryloxy group, a substituted or unsubstituted C₁-C₆₀ heteroarylthio group, —N(Q₁)(Q₂), —Si(Q₃)(Q₄)(Q₅), —Ge(Q₃)(Q₄)(Q₅), —B(Q₆)(Q₇), —P(═O)(Q₈)(Q₉), or —P(Q₈)(Q₉).

In one or more embodiments, R₅ to R₇ may each independently be hydrogen, deuterium, —F, —Cl, —Br, —I, —SF₅, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₁-C₆₀ alkylthio group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryloxy group, a substituted or unsubstituted C₆-C₆₀ arylthio group, a substituted or unsubstituted C₁-C₆₀ heteroaryloxy group, a substituted or unsubstituted C₁-C₆₀ heteroarylthio group, —N(Q₁)(Q₂), —Si(Q₃)(Q₄)(Q₅), —Ge(Q₃)(Q₄)(Q₅), —B(Q₆)(Q₇), —P(═O)(Q₈)(Q₉), or —P(Q₈)(Q₉).

In one or more embodiments, R₁ to R₄ may each independently be hydrogen, deuterium, —F, —Cl, —Br, —I, —SF₅, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₁-C₆₀ alkylthio group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryloxy group, a substituted or unsubstituted C₆-C₆₀ arylthio group, —N(Q₁)(Q₂), —Si(Q₃)(Q₄)(Q₅), —Ge(Q₃)(Q₄)(Q₅), —B(Q₆)(Q₇), —P(═O)(Q₈)(Q₉), or —P(Q₈)(Q₉).

At least one substituent of the substituted C₁-C₆₀ alkyl group, the substituted C₂-C₆₀ alkenyl group, the substituted C₂-C₆₀ alkynyl group, the substituted C₁-C₆₀ alkoxy group, the substituted C₁-C₆₀ alkylthio group, the substituted C₃-C₁₀ cycloalkyl group, the substituted C₁-C₁₀ heterocycloalkyl group, the substituted C₃-C₁₀ cycloalkenyl group, the substituted C₁-C₁₀ heterocycloalkenyl group, the substituted C₆-C₆₀ aryl group, the substituted C₇-C₆₀ alkyl aryl group, the substituted C₇-C₆₀ aryl alkyl group, the substituted C₆-C₆₀ aryloxy group, the substituted C₆-C₆₀ arylthio group, the substituted C₁-C₆₀ heteroaryl group, the substituted C₂-C₆₀ alkyl heteroaryl group, the substituted C₂-C₆₀ heteroaryl alkyl group, the substituted C₁-C₆₀ heteroaryloxy group, the substituted C₁-C₆₀ heteroarylthio group, the substituted monovalent non-aromatic condensed polycyclic group, and the substituted monovalent non-aromatic condensed heteropolycyclic group is:
deuterium, —F, —Cl, —Br, —I, —SF₅, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, or a C₁-C₆₀ alkylthio group;
a C₁-C₆₀ alkyl group, a C₂-C₆₀ alkenyl group, a C₂-C₆₀ alkynyl group, a C₁-C₆₀ alkoxy group, or a C₁-C₆₀ alkylthio group, each substituted with at least one of deuterium, —F, —Cl, —Br, —I, —SF₅, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₇-C₆₀ alkyl aryl group, a C₆-C₆₀ aryloxy group, a C₆-C₆₀ arylthio group, a C₁-C₆₀ heteroaryl group, a C₂-C₆₀ alkyl heteroaryl group, a C₁-C₆₀ heteroaryloxy group, a C₁-C₆₀ heteroarylthio group, a monovalent non-aromatic condensed polycyclic group, a monovalent non-aromatic condensed heteropolycyclic group, —N(Q₁₁)(Q₁₂), —Si(Q₁₃)(Q₁₄)(Q₁₅), —Ge(Q₁₃)(Q₁₄)(Q₁₅), —B(Q₁₆)(Q₁₇), —P(═O)(Q₁₈)(Q₁₉), —P(Q₁₈)(Q₁₉), or a combination thereof;
a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₇-C₆₀ alkyl aryl group, a C₆-C₆₀ aryloxy group, a C₆-C₆₀ arylthio group, a C₁-C₆₀ heteroaryl group, a C₂-C₆₀ alkyl heteroaryl group, a C₁-C₆₀ heteroaryloxy group, a C₁-C₆₀ heteroarylthio group, a monovalent non-aromatic condensed polycyclic group, or a monovalent non-aromatic condensed heteropolycyclic group, each unsubstituted or substituted with at least one of deuterium, —F, —Cl, —Br, —I, —SF₅, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₁-C₆₀ alkylthio group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₇-C₆₀ alkyl aryl group, a C₇-C₆₀ aryl alkyl group, a C₆-C₆₀ aryloxy group, a C₆-C₆₀ arylthio group, a C₁-C₆₀ heteroaryl group, a C₂-C₆₀ alkyl heteroaryl group, a C₂-C₆₀ heteroaryl alkyl group, a C₁-C₆₀ heteroaryloxy group, a C₁-C₆₀ heteroarylthio group, a monovalent non-aromatic condensed polycyclic group, a monovalent non-aromatic condensed heteropolycyclic group, —N(Q₂₁)(Q₂₂), —Si(Q₂₃)(Q₂₄)(Q₂₅), —Ge(Q₂₃)(Q₂₄)(Q₂₅), —B(Q₂₆)(Q₂₇), —P(═O)(Q₂₈)(Q₂₉), —P(Q₂₈)(Q₂₉) or a combination thereof;
—N(Q₃₁)(Q₃₂), —Si(Q₃₃)(Q₃₄)(Q₃₅), —Ge(Q₃₃)(Q₃₄)(Q₃₅), —B(Q₃₆)(Q₃₇), —P(═O)(Q₃₈)(Q₃₉), or —P(Q₃₈)(Q₃₉); or
a combination thereof.

Q₁ to Q₉, Q₁₁ to Q₁₉, Q₂₁ to Q₂₉, and Q₃₁ to Q₃₉ are each independently hydrogen, deuterium, —F, —Cl, —Br, —I, —SF₅, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₁-C₆₀ alkylthio group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₇-C₆₀ alkyl aryl group, a substituted or unsubstituted C₇-C₆₀ aryl alkyl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted C₂-C₆₀ alkyl heteroaryl group, a substituted or unsubstituted C₂-C₆₀ heteroaryl alkyl group, a substituted or unsubstituted C₁-C₆₀ heteroaryloxy group, a substituted or unsubstituted C₁-C₆₀ heteroarylthio group, a substituted or unsubstituted monovalent non-aromatic condensed polycyclic group, or a substituted or unsubstituted monovalent non-aromatic condensed heteropolycyclic group.

In Formula 1, d1, d2, d3, d5, and d6 indicate the number of R₁(s), R₂(s), R₃(s), R₅(s), and R₆(s), respectively, and are each independently a number of 0 to 4 (e.g., 0, 1, 2, 3, or 4). When d1 is 2 or greater, two or more of R₁ may be identical to or different from each other, when d2 is 2 or greater, two or more of R₂ may be identical to or different from each other, when d3 is 2 or greater, two or more of R₃ may be identical to or different from each other, when d5 is 2 or greater, two or more of R₅ may be identical to or different from each other, and when d6 is 2 or greater, two or more of R₆ may be identical to or different from each other.

In Formula 1, d4 indicates the number of R₃(s), and is an integer from 0 to 3 (e.g., 0, 1, 2, or 3). When d4 is 2 or greater, two or more of R₄ may be identical to or different from each other.

In Formula 1, d7 indicates the number of R₇(s), and is an integer from 0 to 5 (e.g., 0, 1, 2, 3, 4, or 5). When d7 is 2 or greater, two or more of R₇ may be identical to or different from each other.

In one or more embodiments, at least one of d2 and d1 may be an integer of 1 or greater. In one or more embodiments, at least one of d2 and d1 may be 4.

In one or more embodiments, R₁ to R₆ may each independently be:
hydrogen, deuterium, —F, —Cl, —Br, —I, —SF₅, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, or a C₁-C₂₀ alkylthio group;
a C₁-C₂₀ alkyl group, a C₁-C₂₀ alkoxy group, or a C₁-C₂₀ alkylthio group, each substituted with at least one of deuterium, —F, —Cl, —Br, —I, —SF₅, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a naphthyl group, a pyridinyl group, a pyrimidinyl group, or a combination thereof;
a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, or an imidazopyrimidinyl group;
a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, or an imidazopyrimidinyl group, each substituted with at least one of deuterium, —F, —Cl, —Br, —I, —SF₅, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a C₁-C₂₀ alkylthio group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, an imidazopyrimidinyl group, —Si(Q₃₃)(Q₃₄)(Q₃₅), or a combination thereof; or
—N(Q₁)(Q₂), —Si(Q₃)(Q₄)(Q₅), —Ge(Q₃)(Q₄)(Q₅), —B(Q₆)(Q₇), —P(═O)(Q₈)(Q₉), or —P(Q₈)(Q₉), and
Q₁ to Q₉ and Q₃₁ to Q₃₃ may each independently be:
—CH₃, —CD₃, —CD₂H, —CDH₂, —CH₂CH₃, —CH₂CD₃, —CH₂CD₂H, —CH₂CDH₂, —CHDCH₃, —CHDCD₂H, —CHDCDH₂, —CHDCD₃, —CD₂CD₃, —CD₂CD₂H, or —CD₂CDH₂;
an n-propyl group, an isopropyl group, an n-butyl group, an isobutyl group, a sec-butyl group, a tert-butyl group, an n-pentyl group, an isopentyl group, a sec-pentyl group, a tert-pentyl group, a phenyl group, or a naphthyl group; or
an n-propyl group, an isopropyl group, an n-butyl group, an isobutyl group, a sec-butyl group, a tert-butyl group, an n-pentyl group, an isopentyl group, a sec-pentyl group, a tert-pentyl group, a phenyl group, or a naphthyl group, each substituted with at least one of deuterium, a C₁-C₁₀ alkyl group, a phenyl group, or a combination thereof.

In one or more embodiments, R₁ to R₇ may each independently be:
hydrogen, deuterium, —F, —Cl, —Br, —I, —SF₅, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, or a C₁-C₂₀ alkylthio group;
a C₁-C₂₀ alkyl group, a C₁-C₂₀ alkoxy group, or a C₁-C₂₀ alkylthio group, each substituted with at least one of deuterium, —F, —Cl, —Br, —I, —SF₅, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a naphthyl group, a pyridinyl group, a pyrimidinyl group, or a combination thereof; or
—N(Q₁)(Q₂), —Si(Q₃)(Q₄)(Q₅), —Ge(Q₃)(Q₄)(Q₅), —B(Q₆)(Q₇), —P(═O)(Q₈)(Q₉), or —P(Q₈)(Q₉), and
Q₁ to Q₉ and Q₃₁ to Q₃₃ may each independently be:
—CH₃, —CD₃, —CD₂H, —CDH₂, —CH₂CH₃, —CH₂CD₃, —CH₂CD₂H, —CH₂CDH₂, —CHDCH₃, —CHDCD₂H, —CHDCDH₂, —CHDCD₃, —CD₂CD₃, —CD₂CD₂H, or —CD₂CDH₂;
an n-propyl group, an isopropyl group, an n-butyl group, an isobutyl group, a sec-butyl group, a tert-butyl group, an n-pentyl group, an isopentyl group, a sec-pentyl group, a tert-pentyl group, a phenyl group, or a naphthyl group; or
an n-propyl group, an isopropyl group, an n-butyl group, an isobutyl group, a sec-butyl group, a tert-butyl group, an n-pentyl group, an isopentyl group, a sec-pentyl group, a tert-pentyl group, a phenyl group, or a naphthyl group, each substituted with at least one of deuterium, a C₁-C₁₀ alkyl group, a phenyl group, or a combination thereof.

In one or more embodiments, R₁ to R₆ may each independently be:
hydrogen, deuterium, —F, —Cl, —Br, —I, —SF₅, —CD₃, —CD₂H, —CDH₂, —CF₃, —CF₂H, —CFH₂, a C₁-C₆₀ alkyl group, a C₂-C₆₀ alkenyl group, a C₂-C₆₀ alkynyl group, a C₁-C₆₀ alkoxy group, or a C₁-C₆₀ alkylthio group; or
a group represented by one of Formulae 9-1 to 9-61, 9-201 to 9-240, 10-1 to 10-129, or 10-201 to 10-355; or
—N(Q₁)(Q₂), —Si(Q₃)(Q₄)(Q₅) or —Ge(Q₃)(Q₄)(Q₅), and
Q₁ to Q₅ are each as described herein:

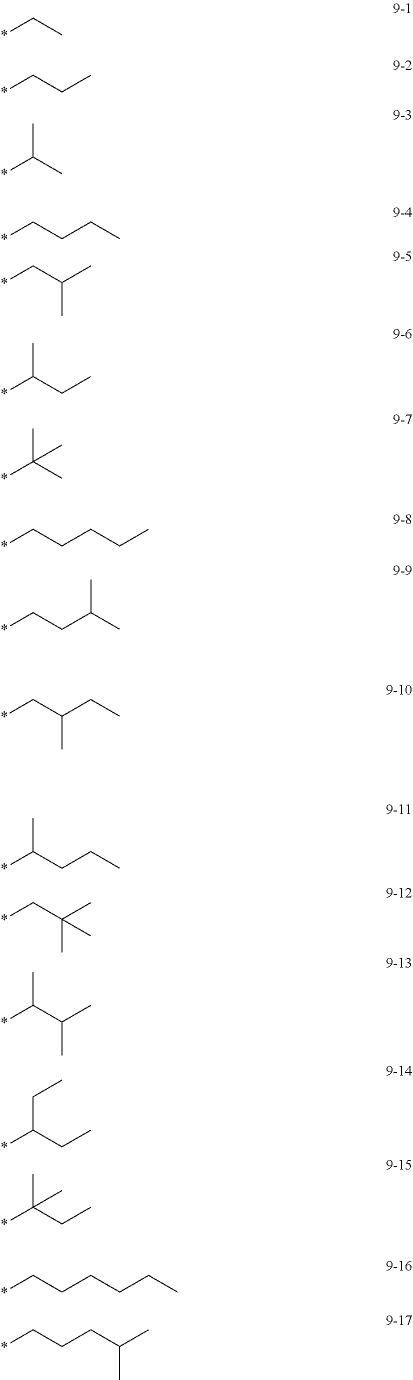

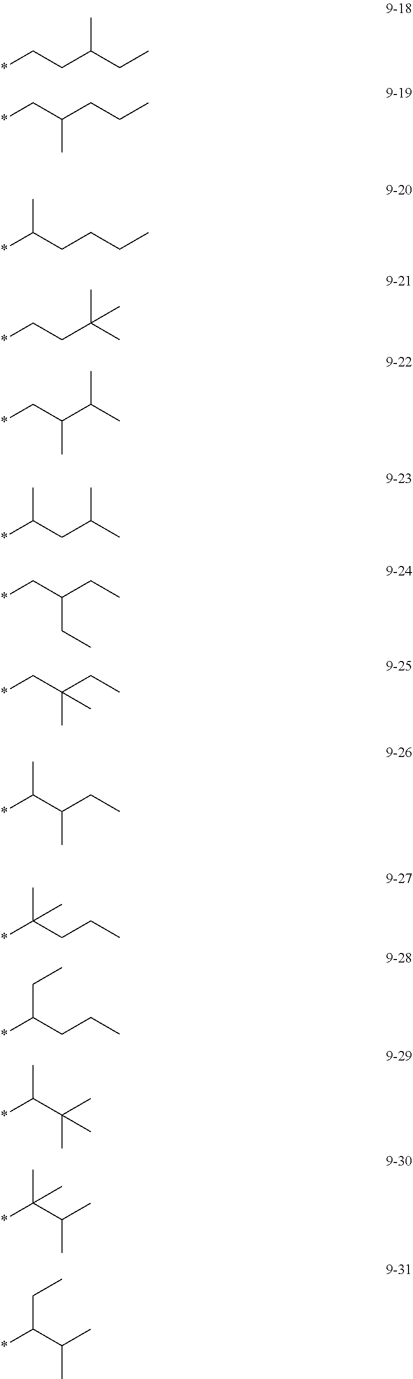

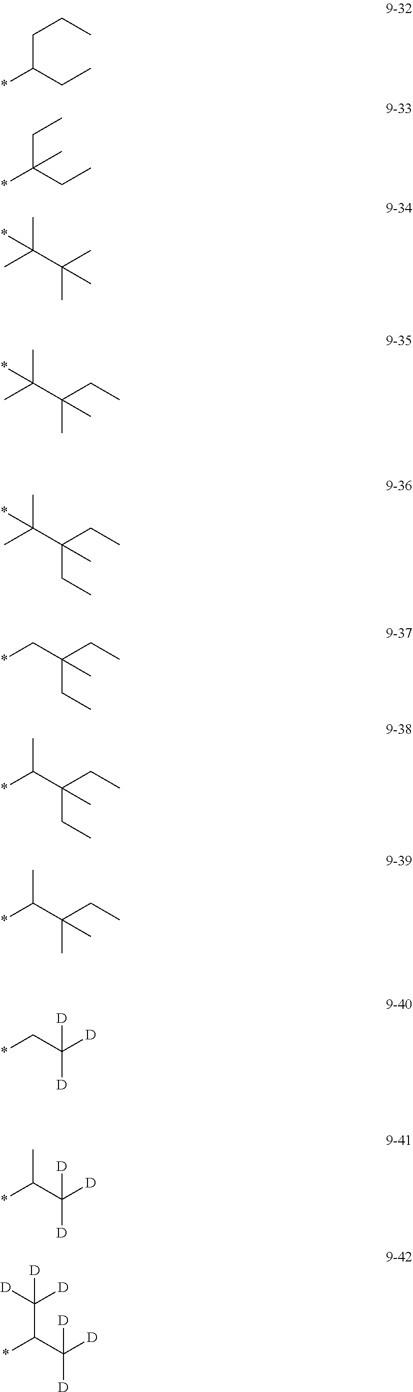

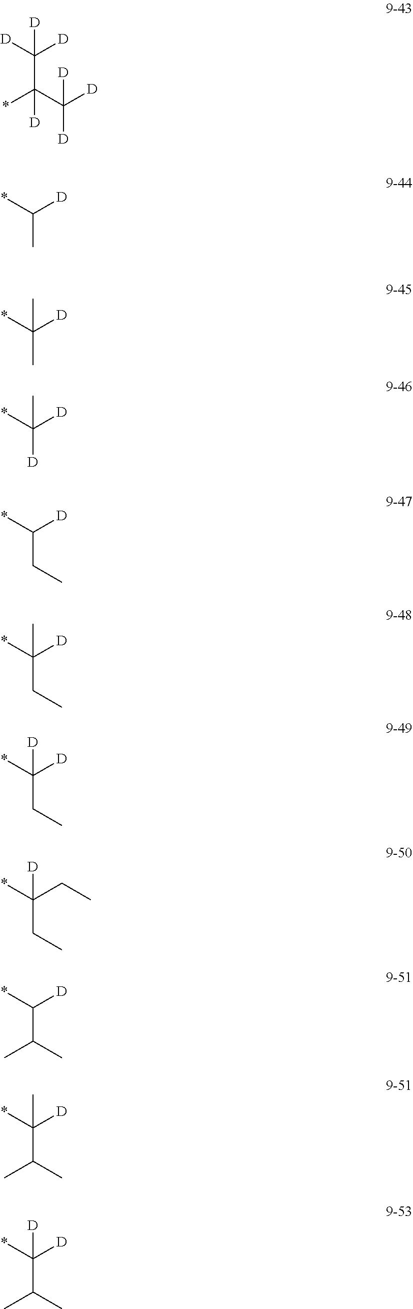

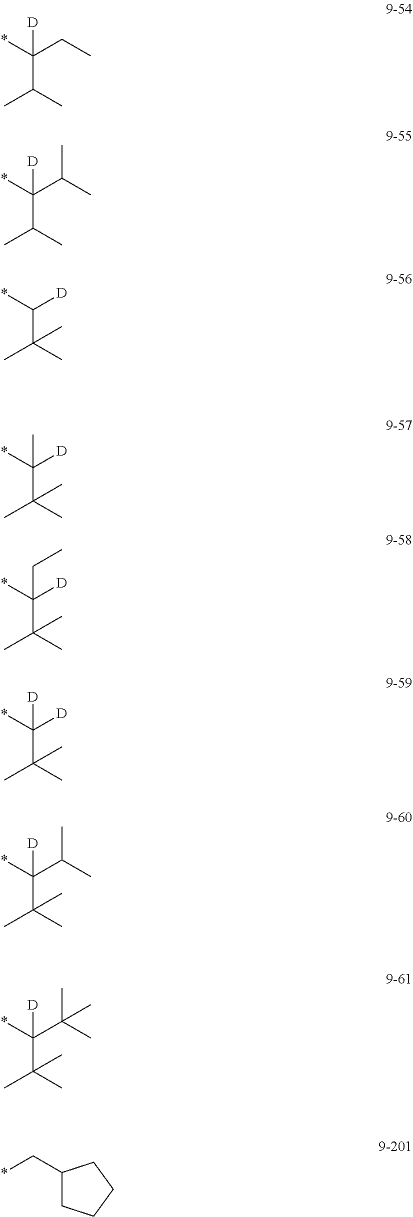

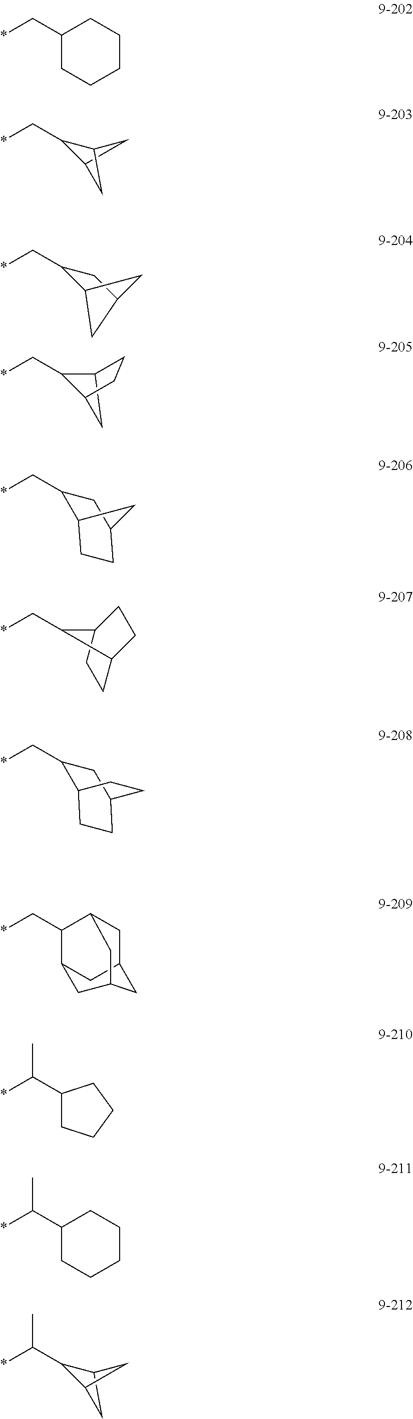

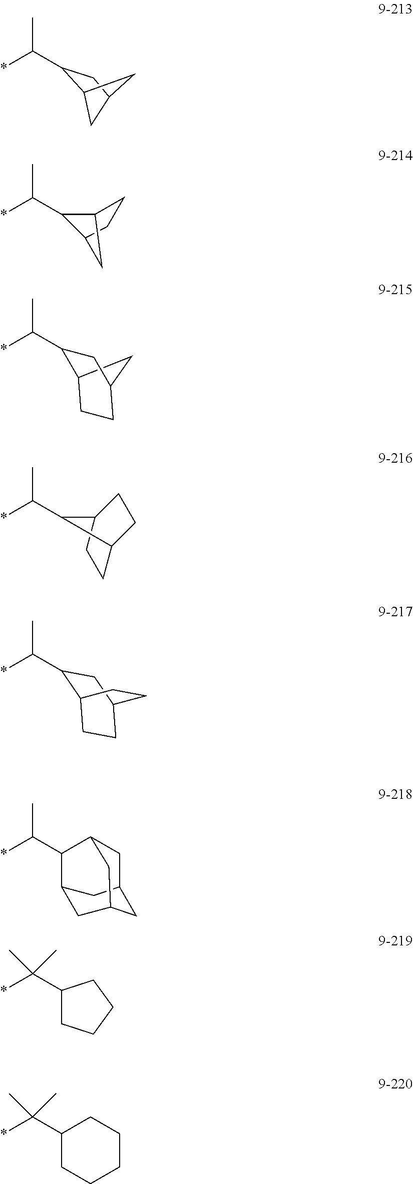

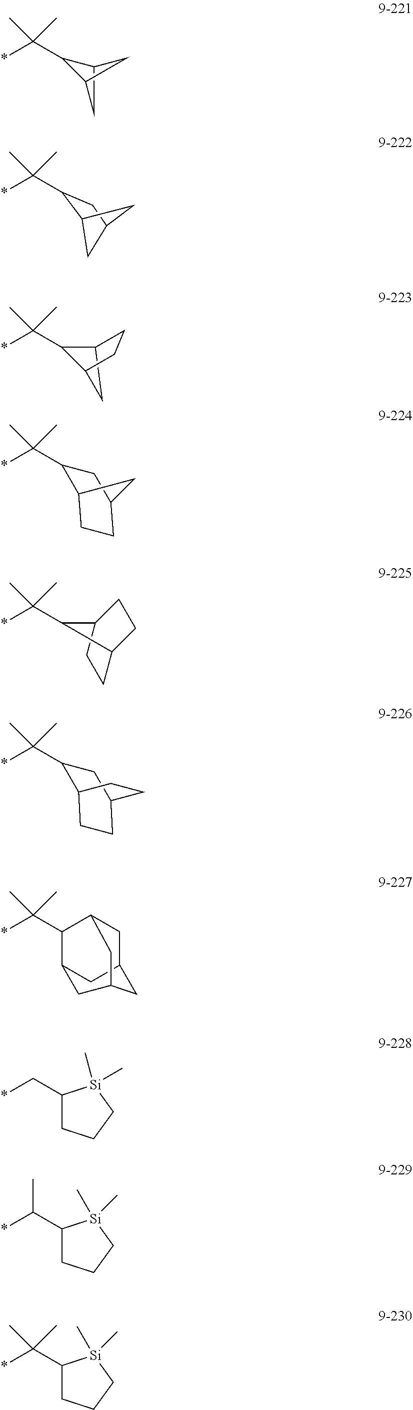

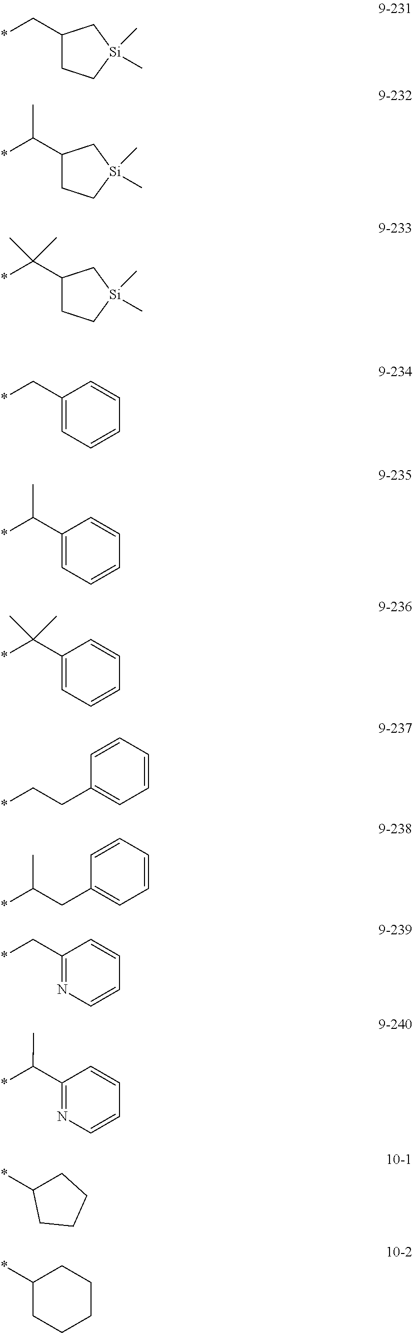

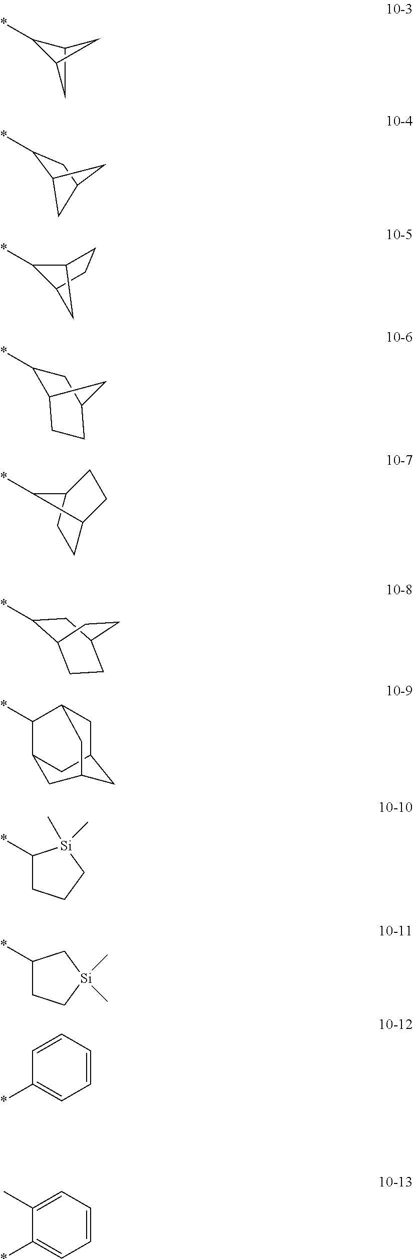

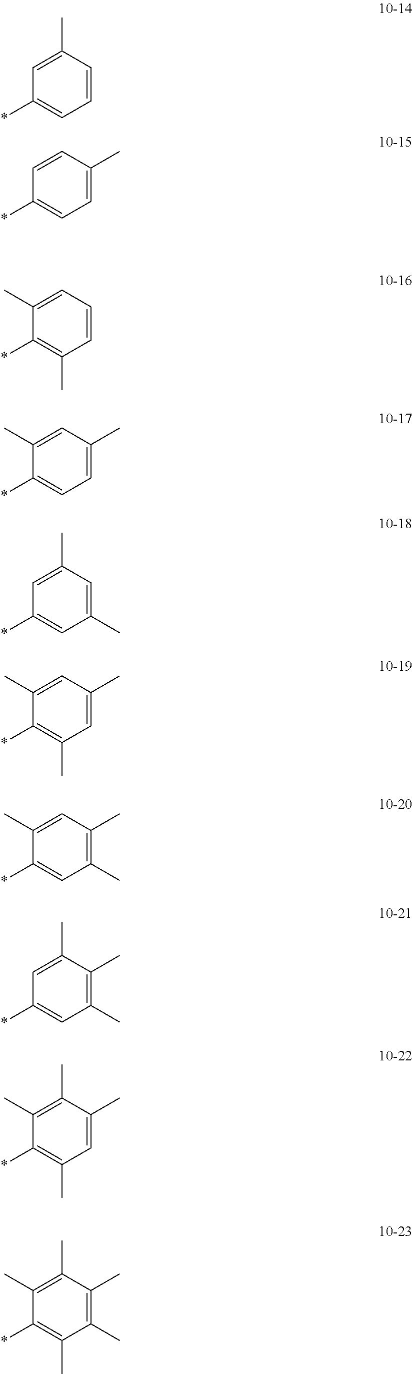

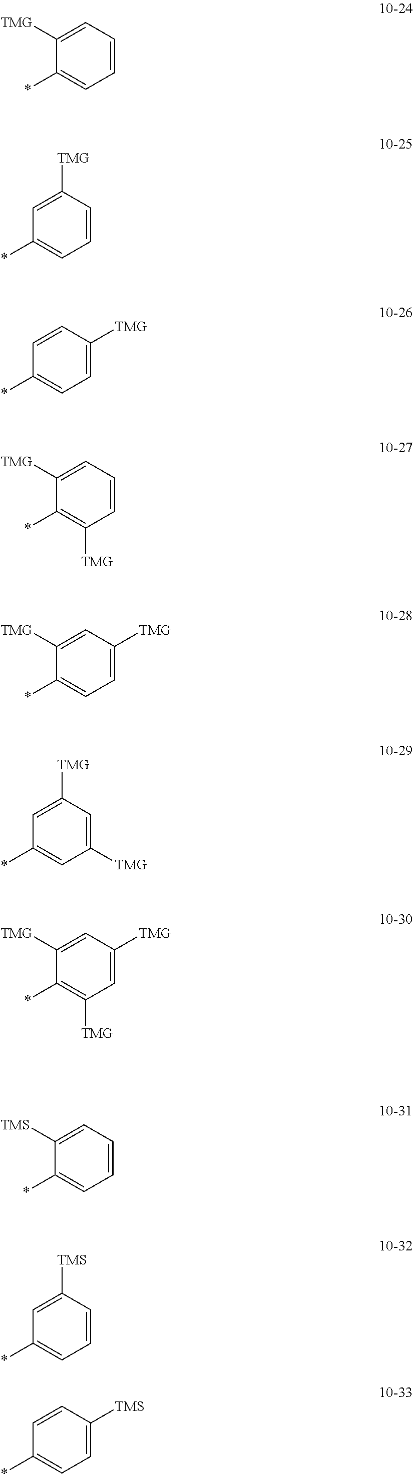

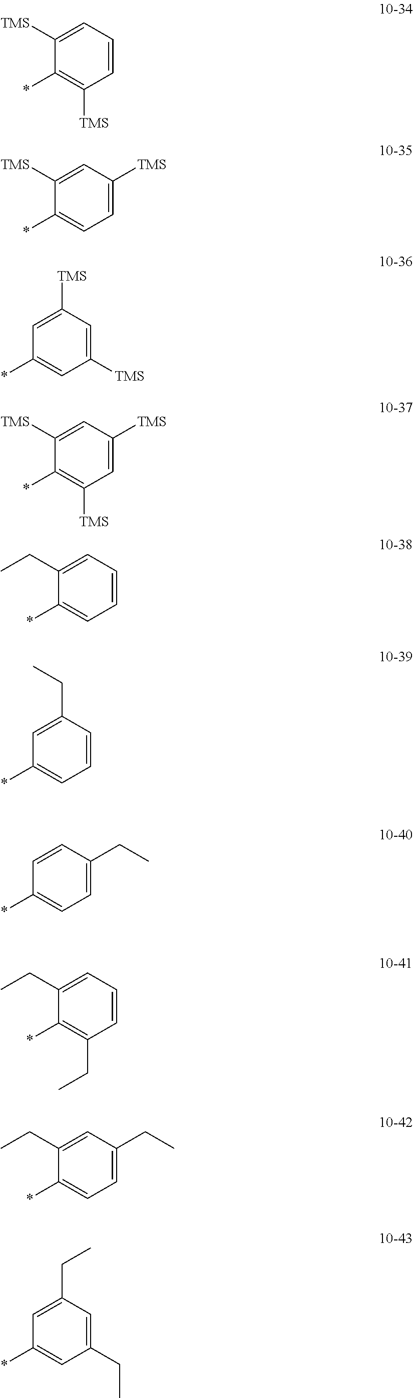

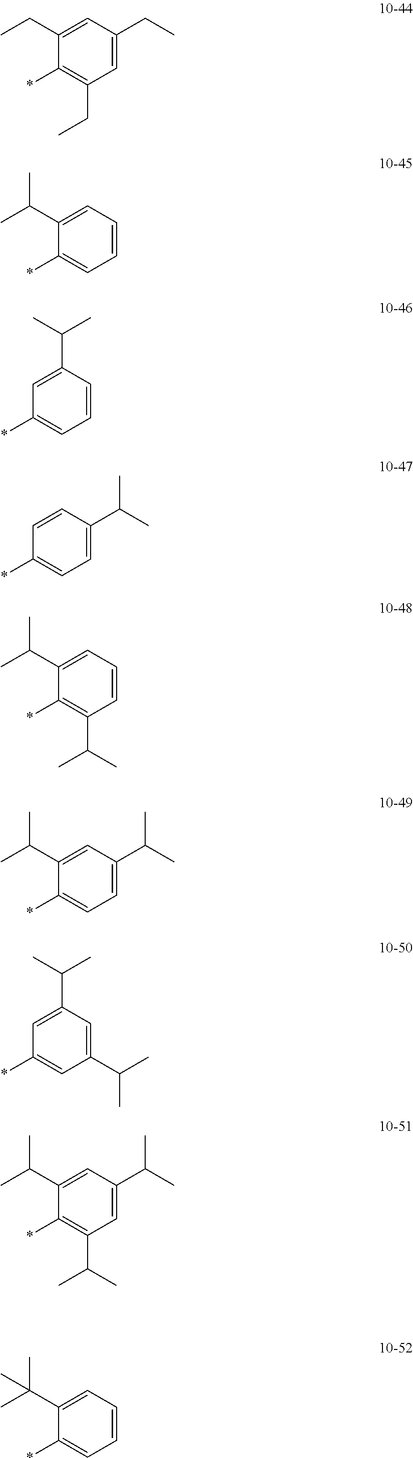

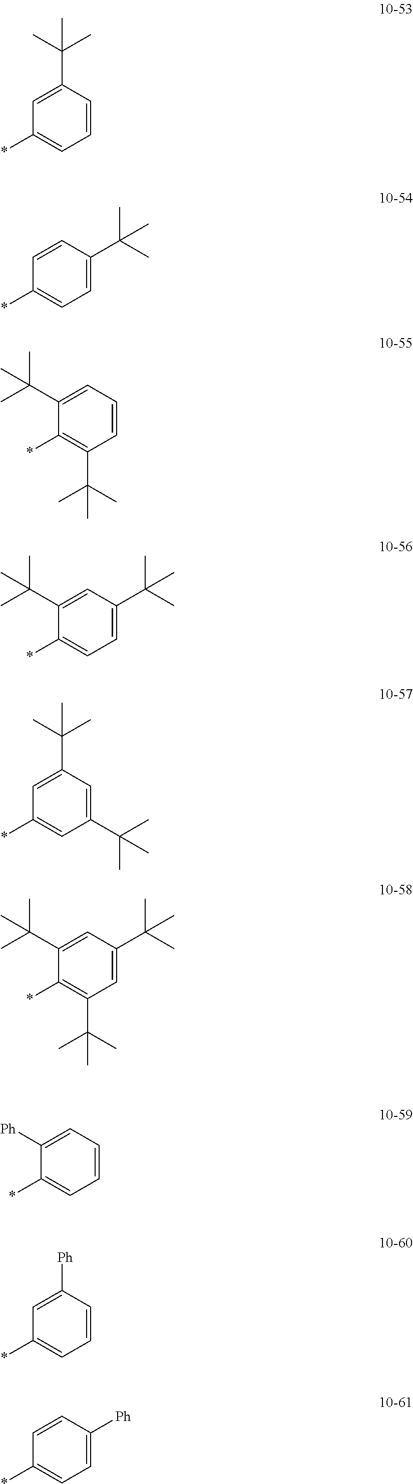

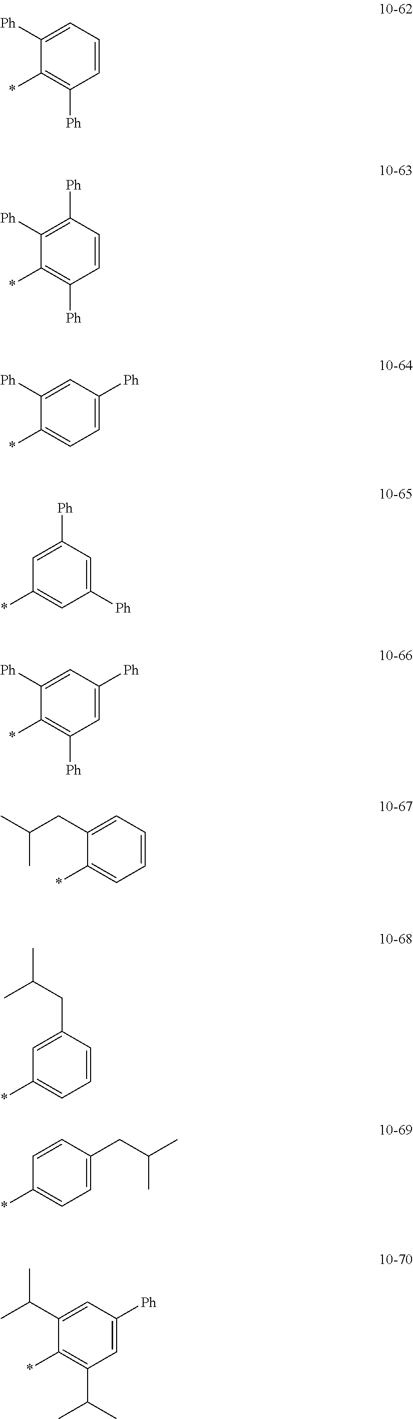

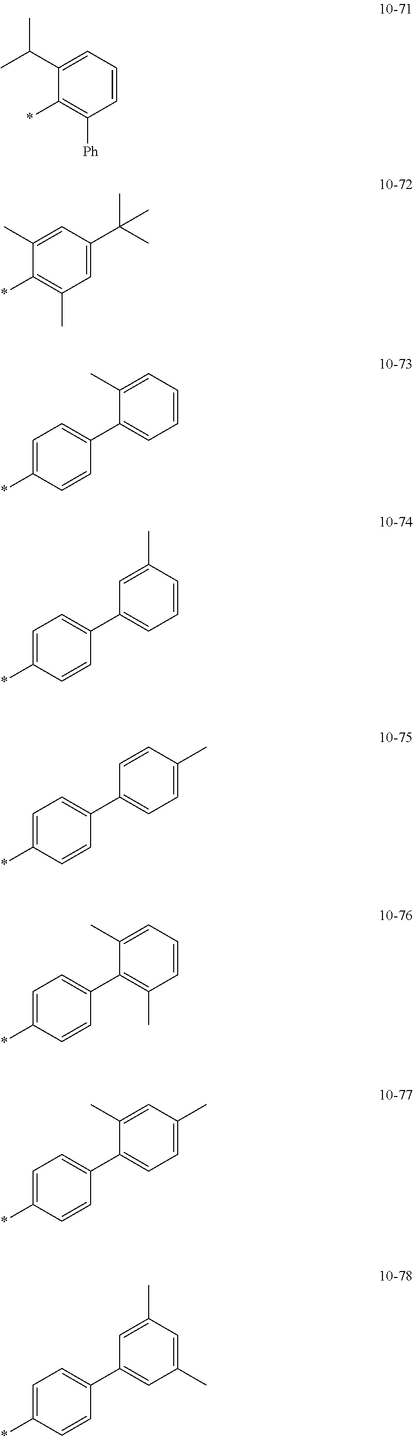

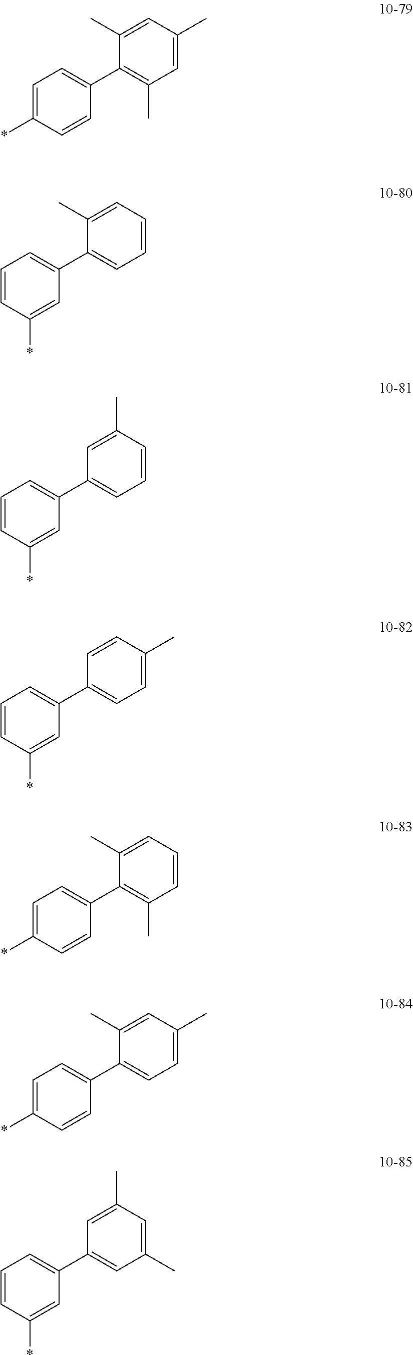

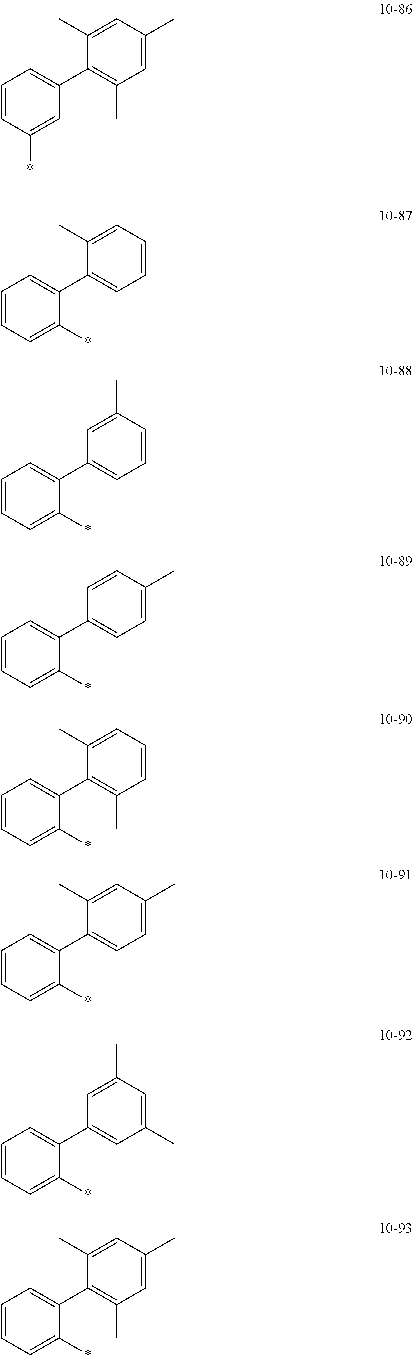

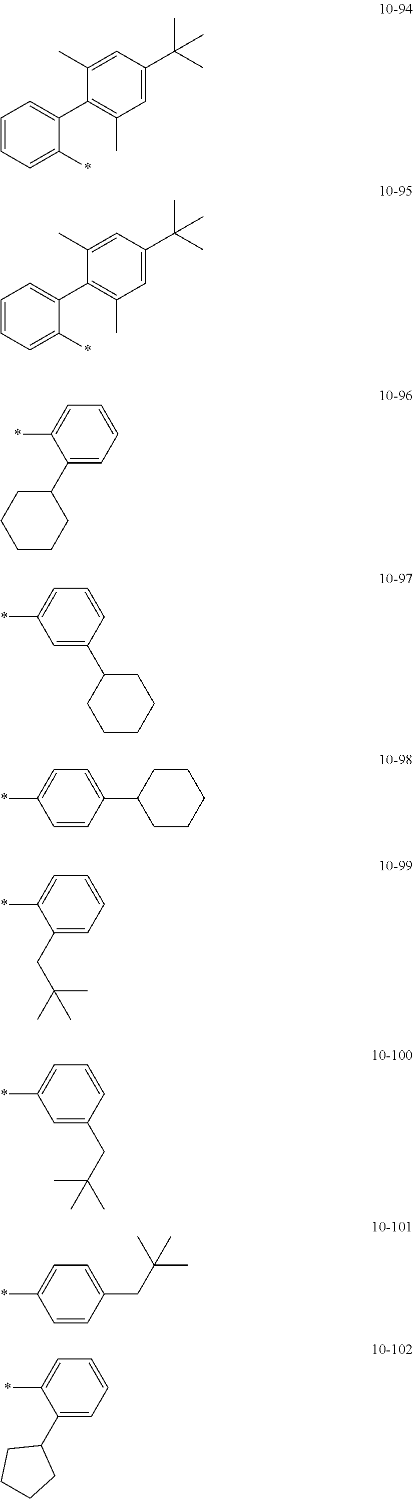

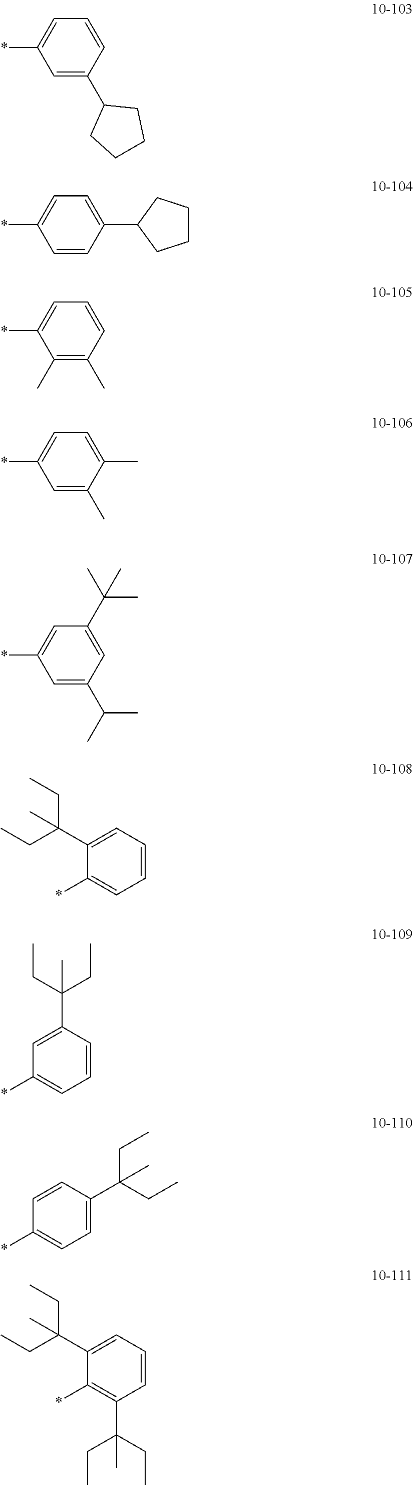

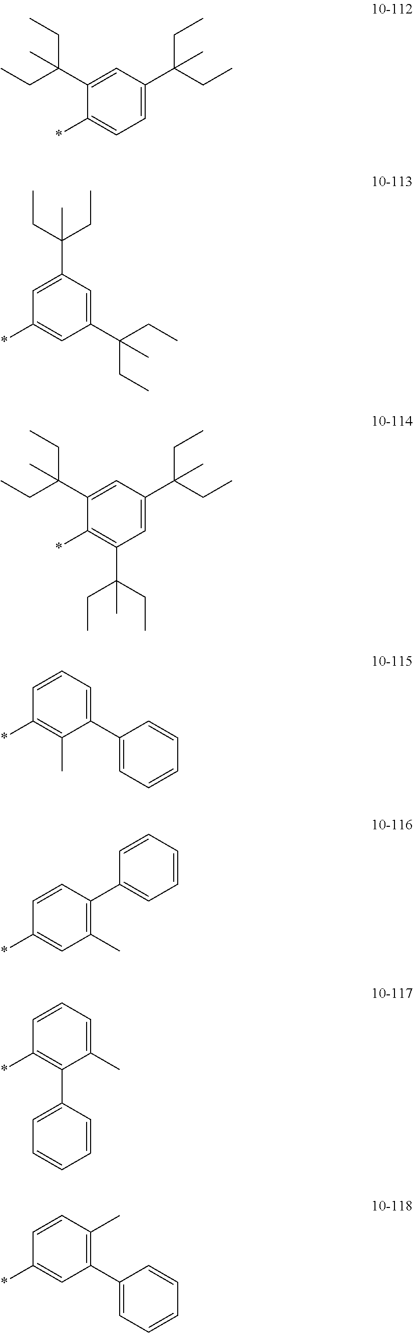

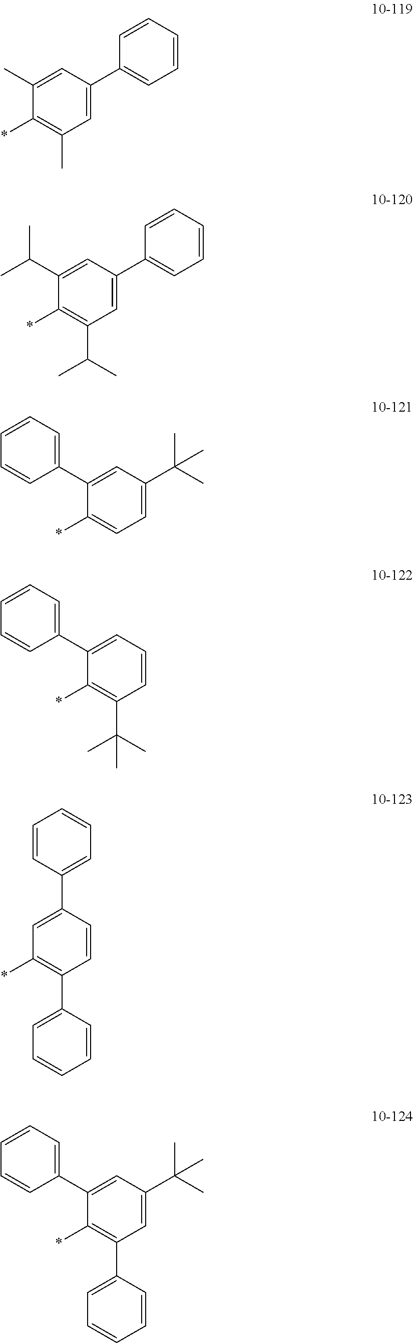

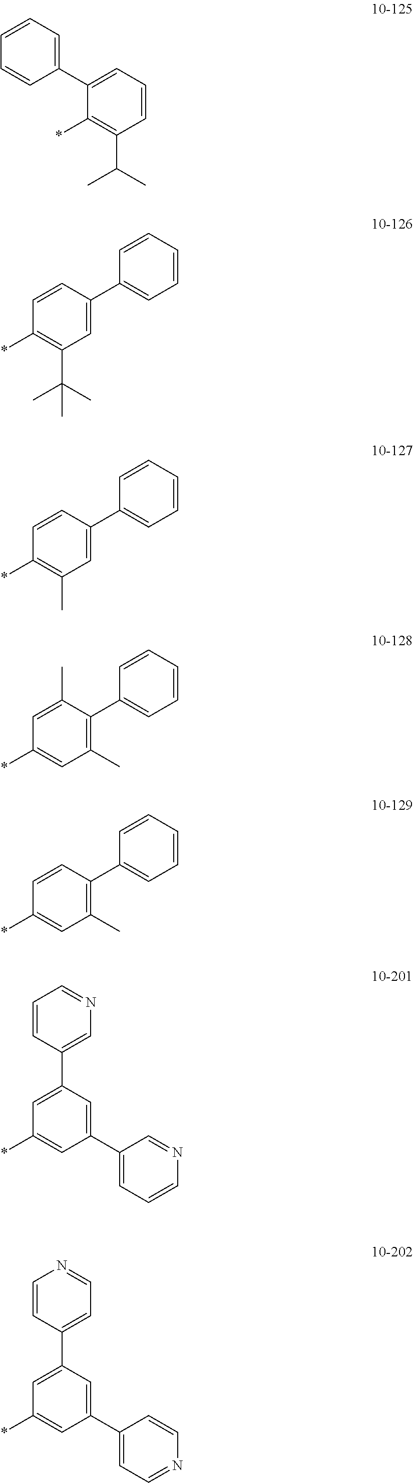

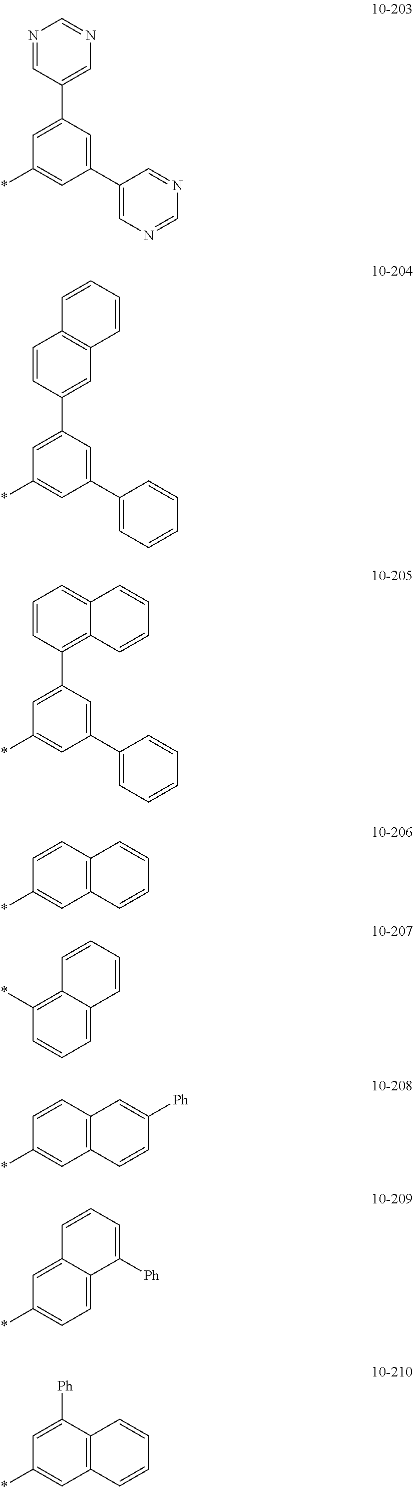

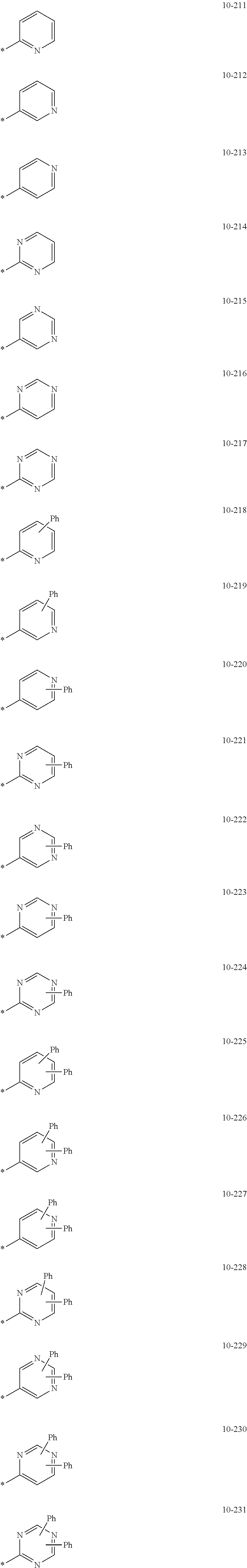

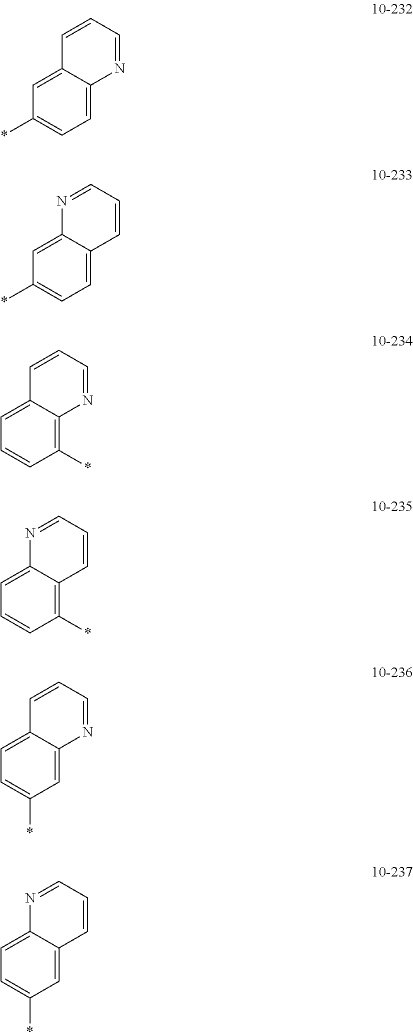

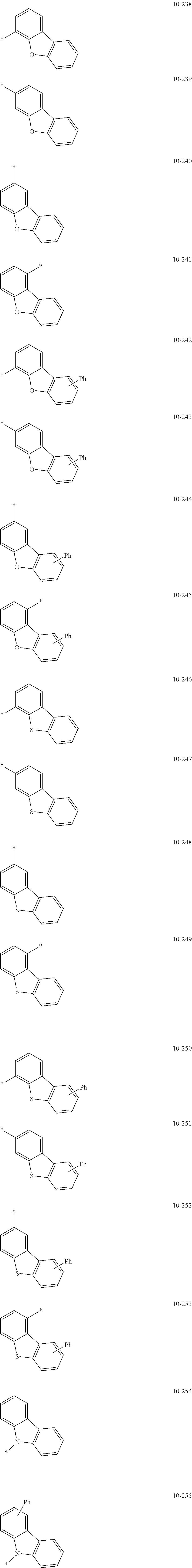

[chemical structure]

[chemical structure]

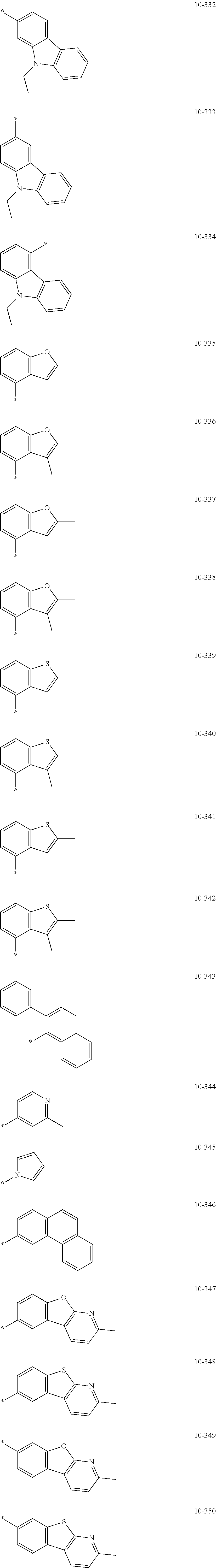

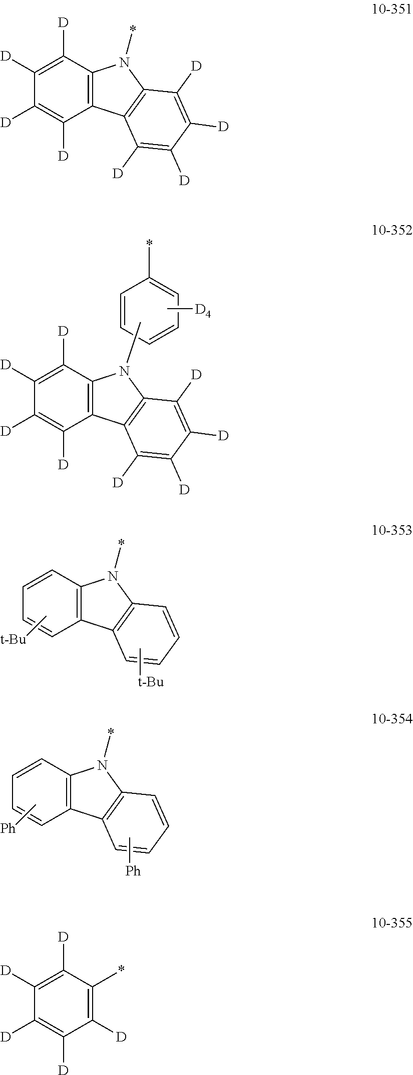

wherein, in Formulae 9-1 to 9-61, 9-201 to 9-240, 10-1 to 10-129, and 10-201 to 10-355, * indicates a binding site to a neighboring atom, “Ph” represents a phenyl group, “TMS” represents a trimethylsilyl group, “TMG” represents a trimethylgermyl group, and “t-Bu” represents a t-butyl group.

In one or more embodiments, R₁ to R₆ may each independently be:
hydrogen, deuterium, —F, —Cl, —Br, —I, —SF₅, —CD₃, —CD₂H, —CDH₂, —CF₃, —CF₂H, —CFH₂, a C₁-C₆₀ alkyl group, a C₂-C₆₀ alkenyl group, a C₂-C₆₀ alkynyl group, a C₁-C₆₀ alkoxy group, or a C₁-C₆₀ alkylthio group; or
a group represented by one of Formulae 9-1 to 9-61 or 9-201 to 9-240; or
—N(Q₁)(Q₂), —Si(Q₃)(Q₄)(Q₅) or —Ge(Q₃)(Q₄)(Q₅), and
Q₁ to Q₅ are each as described herein:

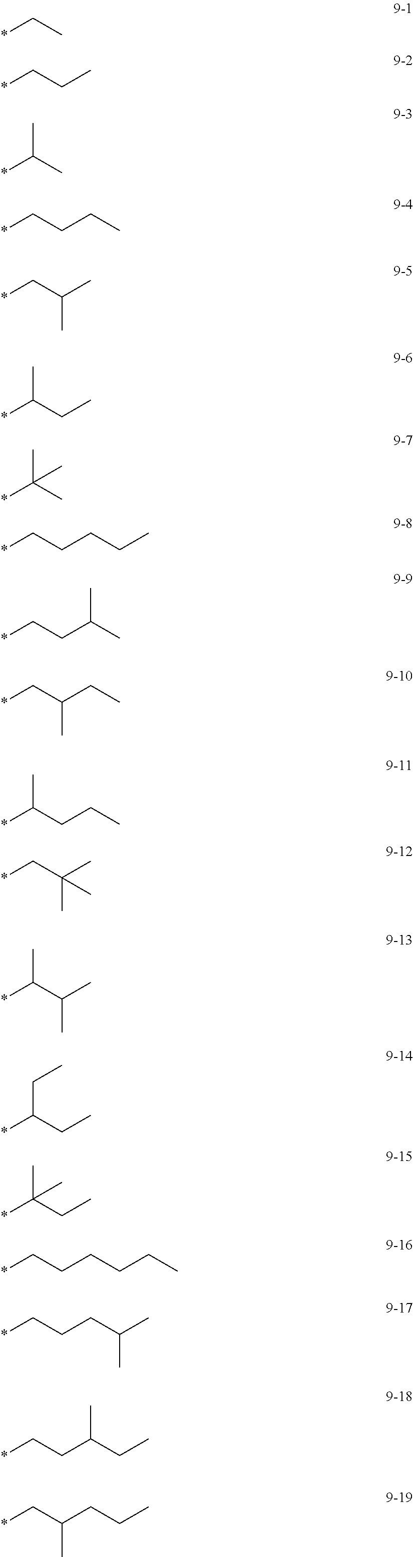

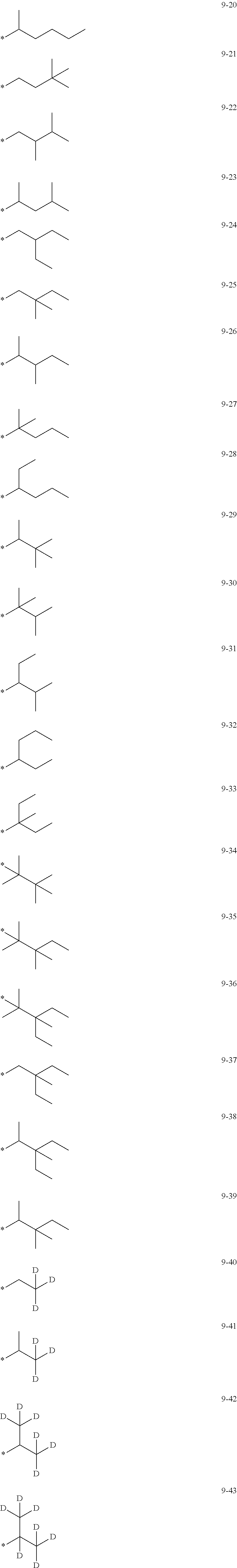

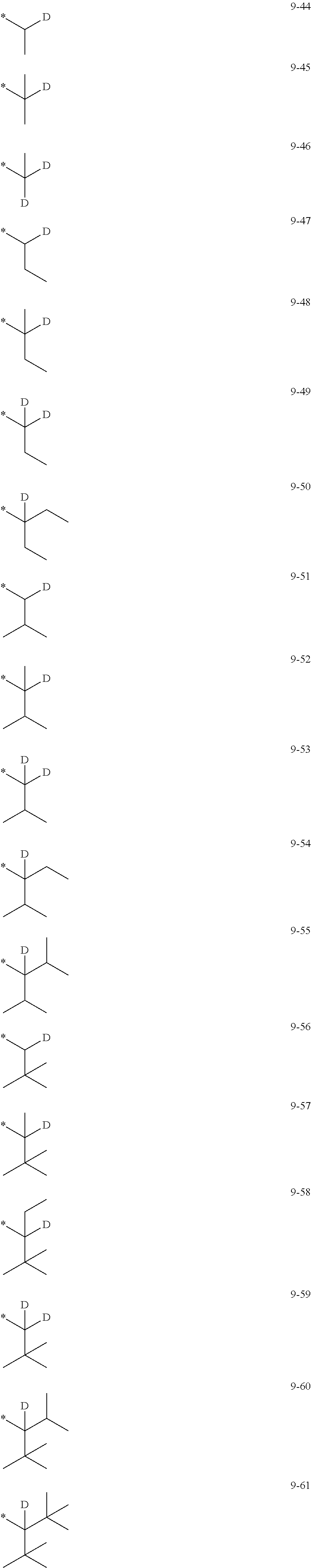

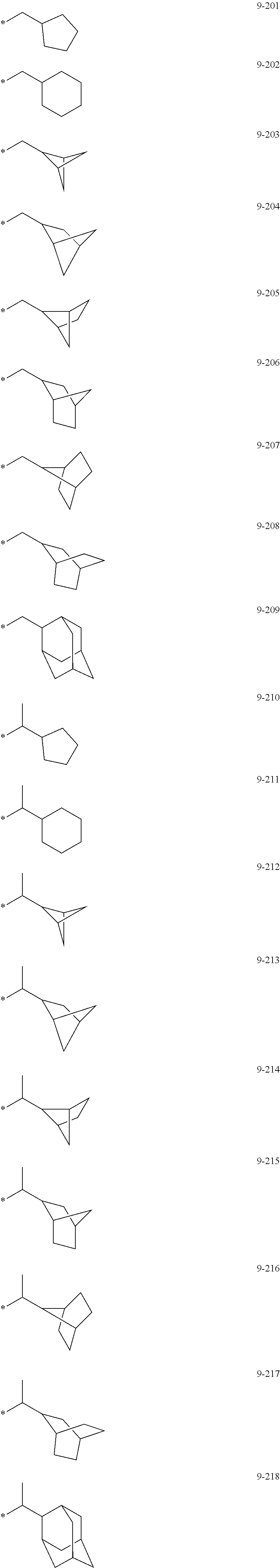

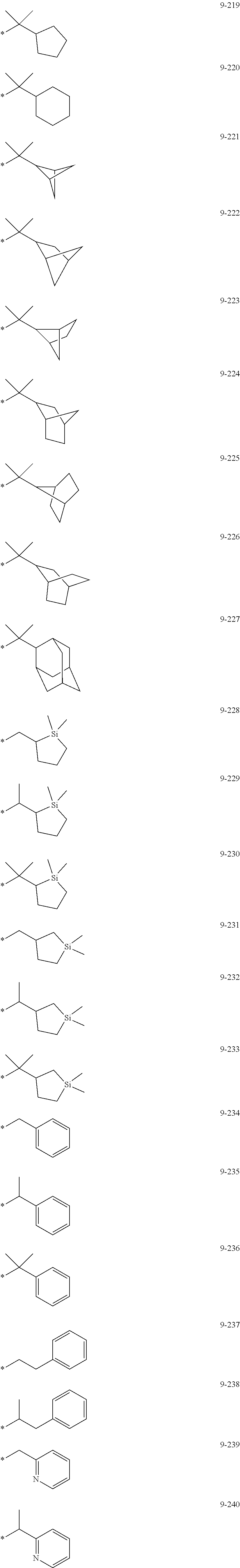

wherein, in Formulae 9-1 to 9-61 and 9-201 to 9-240, * indicates a binding site to a neighboring atom.

The heterocyclic compound represented by Formula 1 includes at least one deuterium.

In one or more embodiments, the heterocyclic compound may include deuterium as a substituent or may include deuterium as a part of a substituent group (i.e., a group that is substituted with at least one deuterium). For example, one or more of R₁ to R₇ may be deuterium, and/or one or more of R₁ to R₇ may be a group that is substituted with at least one deuterium (for example, —CH₂D, —CHD₂, —CD₃, or the like).

In one or more embodiments, the heterocyclic compound represented by Formula 1 may satisfy at least one of Conditions 1 to 7:
Condition 1
at least one R₁(s) is deuterium;
Condition 2
at least one R₂ is deuterium;
Condition 3
at least one R₃ is deuterium;
Condition 4
at least one R₄ is deuterium;
Condition 5
at least one of R₅ is deuterium;
Condition 6
at least one of R₆ is deuterium;
Condition 7
at least one of R₇ is deuterium.

For example, the heterocyclic compound may satisfy at least one of Conditions 1 and 2.

In one or more embodiments, the heterocyclic compound may satisfy at least two of Conditions 1 to 7.

In one or more embodiments, the heterocyclic compound may satisfy at least three of Conditions 1 to 7.

In one or more embodiments, the heterocyclic compound may satisfy at least four of Conditions 1 to 7.

In one or more embodiments, the heterocyclic compound may satisfy at least five of Conditions 1 to 7.

In one or more embodiments, the heterocyclic compound may satisfy at least six of Conditions 1 to 7.

In one or more embodiments, the heterocyclic compound may satisfy all of Conditions 1 to 7.

In one or more embodiments, the heterocyclic compound represented by Formula 1 may satisfy at least one of Conditions 1′ to 7′:
Condition 1′
each R₁ is deuterium, and
d1 is 4;
Condition 2′
each R₂ is deuterium, and
d2 is 4;
Condition 3′
each R₃ is deuterium, and
d3 is 4;
Condition 4′
each R₄ is deuterium, and
d4 is 3;
Condition 5′
each R₅ is deuterium, and
k1 is 1, and d5 is 4; or k1 is 2, and d5 is 3;
Condition 6′
each R₆ is deuterium, and
d6 is 4;
Condition 7′
each R₇ is deuterium, and
d7 is 4.

In one or more embodiments, the heterocyclic compound may satisfy at least two of Conditions 1′ to 7′.

In one or more embodiments, the heterocyclic compound may satisfy at least three of Conditions 1′ to 7′.

In one or more embodiments, the heterocyclic compound may satisfy at least four of Conditions 1′ to 7′.

In one or more embodiments, the heterocyclic compound may satisfy at least five of Conditions 1′ to 7′.

In one or more embodiments, the heterocyclic compound may satisfy at least six of Conditions 1′ to 7′.

In one or more embodiments, the heterocyclic compound may satisfy all of Conditions 1′ to 7′.

In one or more embodiments, the substitution ratio of deuterium of the heterocyclic compound represented by Formula 1 may be greater than about 0% and less than or equal to about 100%.

In one or more embodiments, the substitution ratio of deuterium of the heterocyclic compound represented by Formula 1 may be greater than or equal to about 10% and less than or equal to about 100%. For example, the substitution ratio of deuterium may be greater than or equal to about 10% and less than or equal to about 100%, greater than or equal to about 10% and less than or equal to about 90%, greater than or equal to about 15% and less than or equal to about 100%, greater than or equal to about 15% and less than or equal to about 90%, greater than or equal to about 20% and less than or equal to about 100%, greater than or equal to about 20% and less than or equal to about 90%, greater than or equal to about 40% and less than or equal to about 100%, or greater than or equal to about 40% and less than or equal to about 90%.

In one or more embodiments, the substitution ratio of deuterium of heterocyclic compound may be greater than or equal to about 40% and less than or equal to about 90%.

The term “substitution ratio of deuterium of the heterocyclic compound represented by Formula 1” as used herein refers to the ratio of a number of substituents substituted with deuterium to the number of substituents that can be substituted in the core of the heterocyclic compound represented by Formula 1. That is, the substitution rate of deuterium refers to, in Formulae 1 and A, the ratio of the number of deuterium in R₁ to R₇ to the total number of R₁ to R₇.

In one or more embodiments, among R₁, R₂, R₃, R₄, R₅, R₆, and R₇, at least about 10% of the substituents may be deuterium.

In one or more embodiments, among R₁, R₂, R₃, R₄, R₅, R₆, and R₇, at least about 20% of the substituents may be deuterium.

In one or more embodiments, among R₁, R₂, R₃, R₄, R₅, R₆, and R₇, at least about 30% of the substituents may be deuterium.

In one or more embodiments, among R₁, R₂, R₃, R₄, R₅, R₆, and R₇, at least about 40% of the substituents may be deuterium.

In one or more embodiments, among R₁, R₂, R₃, R₄, R₅, R₆, and R₇, at least about 80% of the substituents may be deuterium.

In one or more embodiments, among R₁, R₂, R₃, R₄, R₅, R₆, and R₇, at least about 90% of the substituents may be deuterium.

In one or more embodiments, the heterocyclic compound represented by Formula 1 may be represented by Formula 1A:

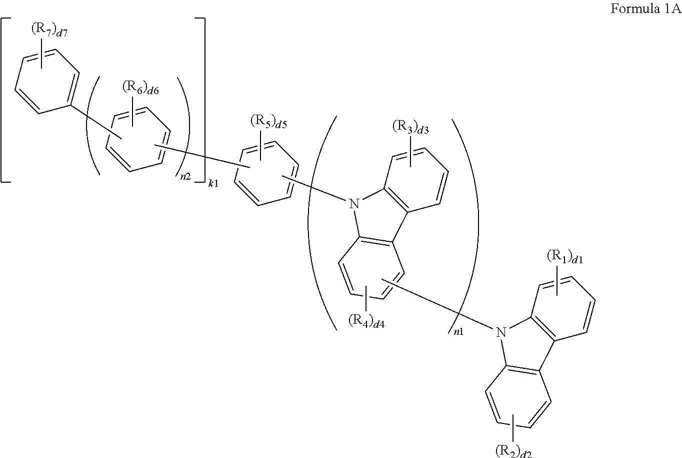

wherein, in Formula 1A,
k1, n1, n2, R₁ to R₇, and d1 to d7 are each as described herein.

In one or more embodiments, a moiety represented by

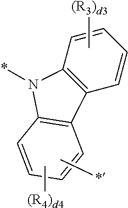

in Formula 1 may be a group represented by one of Formulae 2-1 to 2-4:

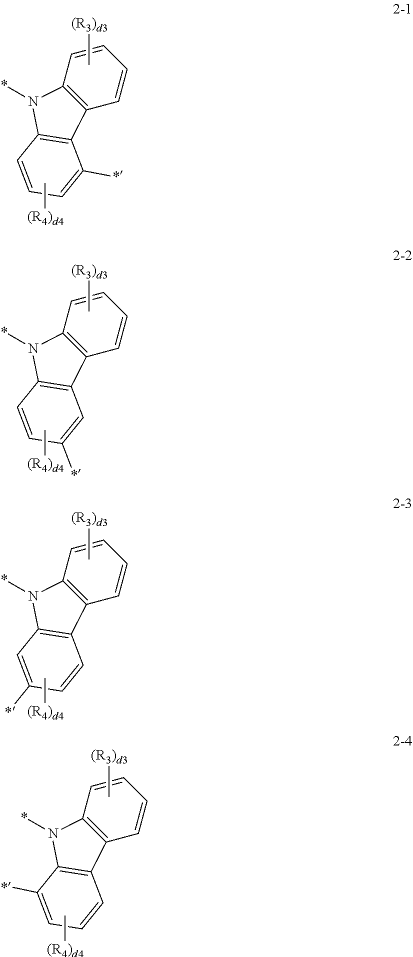

wherein, in Formulae 2-1 to 2-4,
R₃, R₄, d3, and d4 are each as described herein, and
* and *′ each indicate a binding site to a neighboring atom.

In one or more embodiments, a moiety represented by

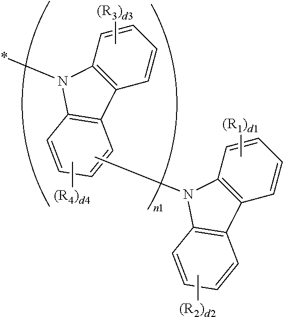

in Formula 1 may be a group represented by one of Formulae 3-1 to 3-16:

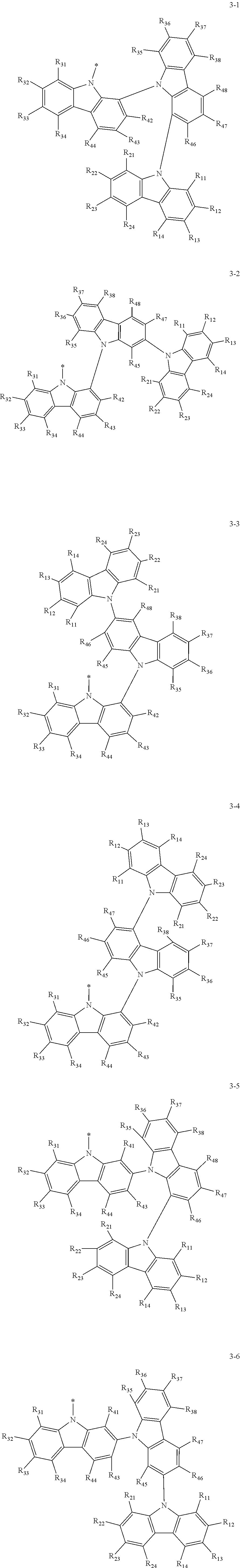

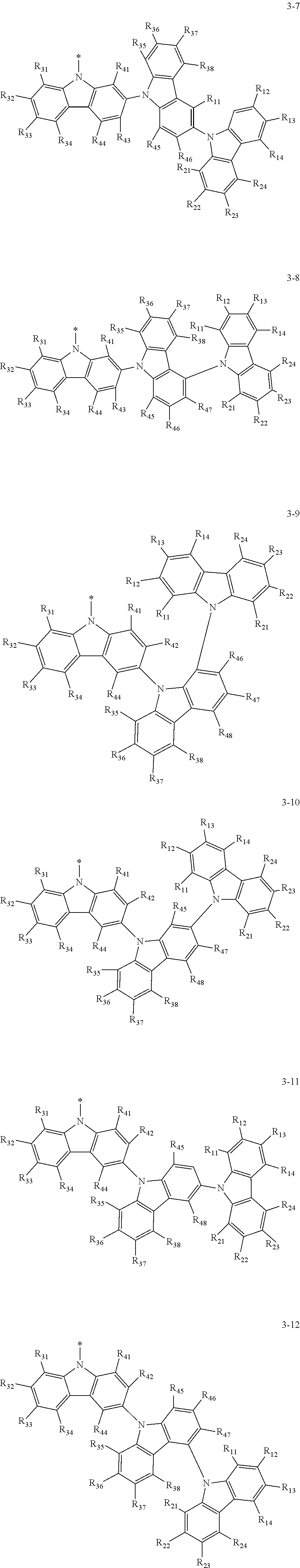

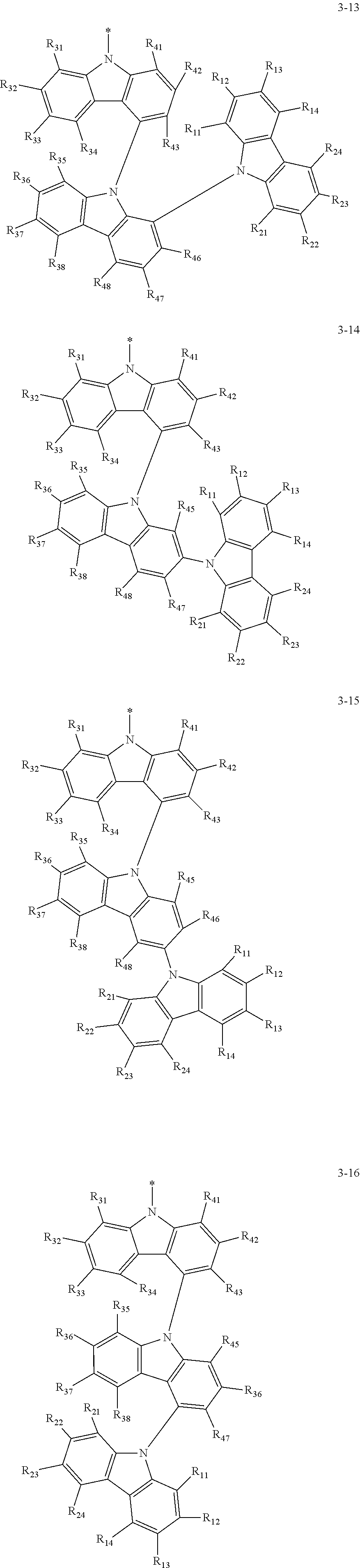

wherein, in Formulae 3-1 to 3-16
R₁₁ to R₁₄ are each as described in connection with R₁,
R₂₁ to R₂₄ are each as described in connection with R₂,
R₃₁ to R₃₈ are each as described in connection with R₃,
R₄₁ to R₄₈ are each as described in connection with R₄, and
* indicates a binding site to a neighboring atom.

In one or more embodiments, in Formulae 3-1 to 3-4, at least one of R₁₁ to R₁₄, R₂₁ to R₂₄, R₃₁ to R₃₄, and R₄₁ to R₄₄ may be deuterium.

In one or more embodiments, in Formulae 3-1 to 3-4, some or all of R₁₁ to R₁₄, R₂₁ to R₂₄, R₃₁ to R₃₄, and R₄₁ to R₄₄ may be deuterium.

In one or more embodiments, in Formulae 3-5 to 3-16, at least one of R₁₁ to R₁₄, R₂₁ to R₂₄, R₃₁ to R₃₈, and R₄₁ to R₄₈ may be deuterium.

In one or more embodiments, in Formulae 3-5 to 3-16, some or all of R₁₁ to R₁₄, R₂₁ to R₂₄, R₃₁ to R₃₈, and R₄₁ to R₄₈ may be deuterium.

In one or more embodiments, the group represented by Formula A may be a group represented by one of Formulae A-1 to A-13:

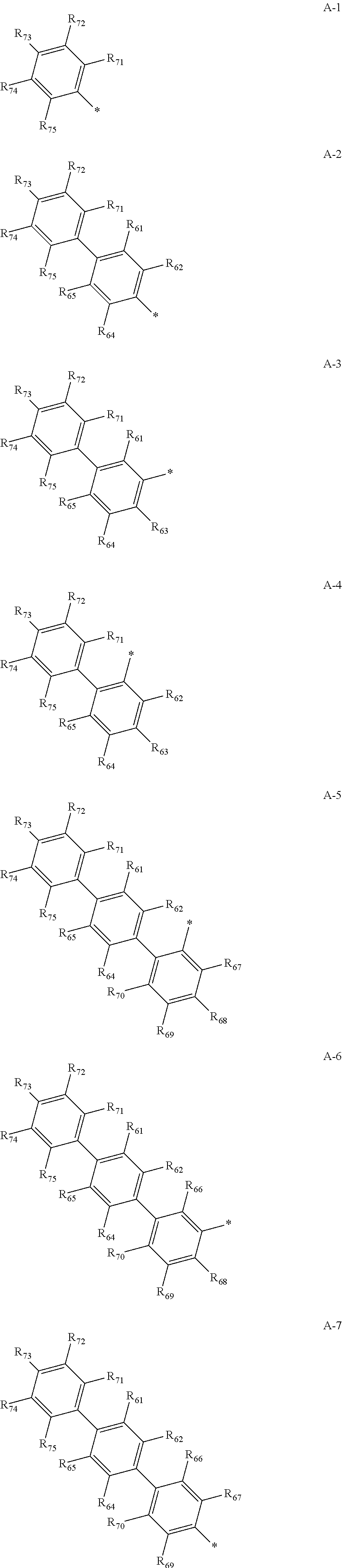

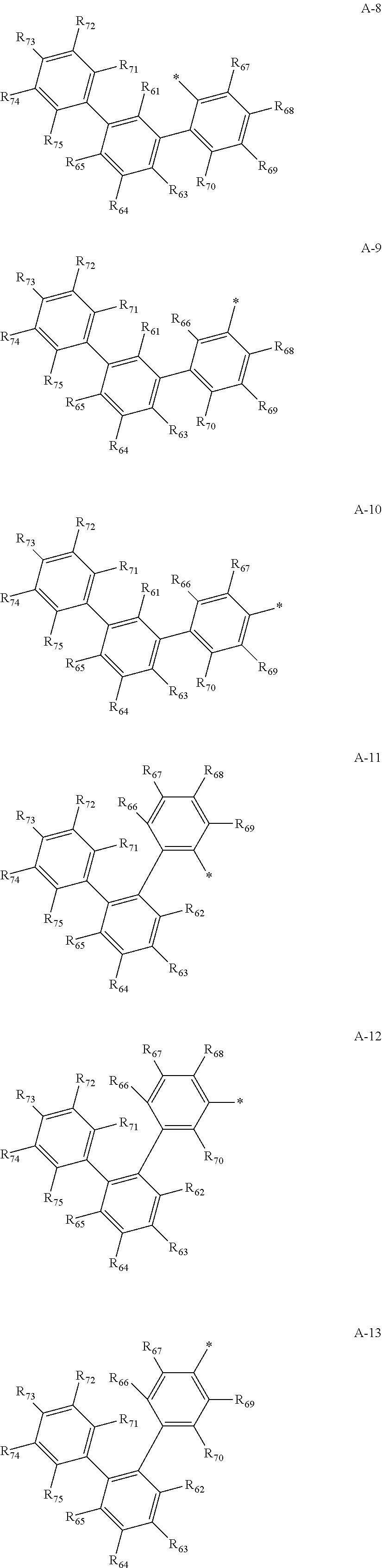

wherein, in Formulae A-1 to A-13,
R₇₁ to R₇₅ are each as described in connection with R₇, and
R₆₁ to R₇₀ each as described in connection with R₆, and
* indicates a binding site to a neighboring atom.

In one or more embodiments, in Formulae A-1 to A-4, at least one of R₆₁ to R₆₅ and R₇₁ to R₇₅ may be deuterium.

In one or more embodiments, in Formulae A-1 to A-4, some or all of R₆₁ to R₆₅ and R₇₁ to R₇ s may be deuterium.

In one or more embodiments, in Formulae A-5 to A-13, at least one of R₆₁ to R₇₀ and R₇₁ to R₇₅ may be deuterium.

In one or more embodiments, in Formulae A-5 to A-13, some or all of R₆₁ to R₆₅ and R₇₁ to R₇₅ may be deuterium.

In one or more embodiments, a moiety represented by

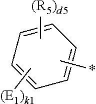

in Formula 1 may be a group represented by one of Formulae 4-1 to 4-17:

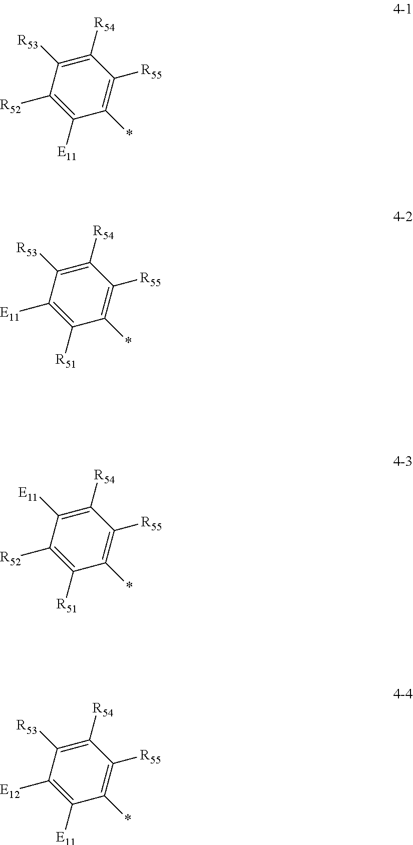

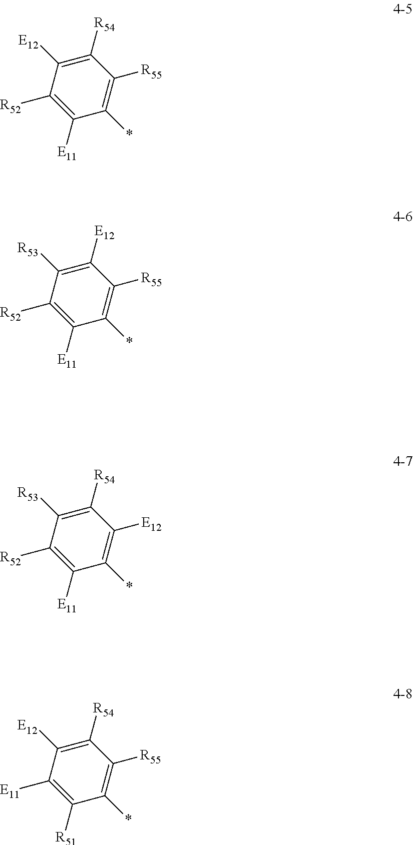

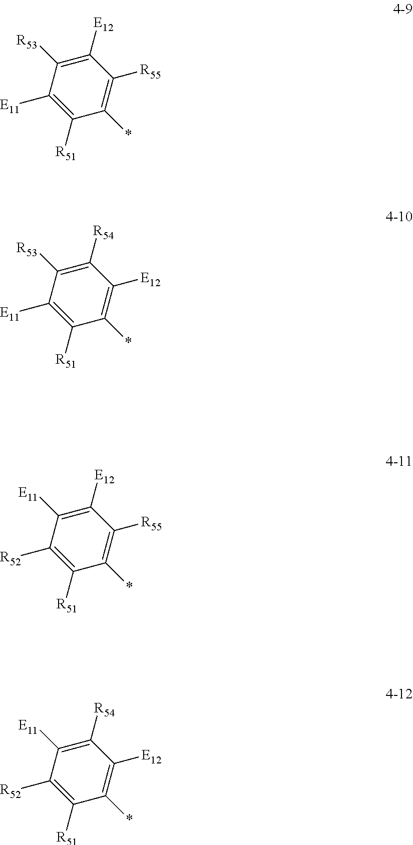

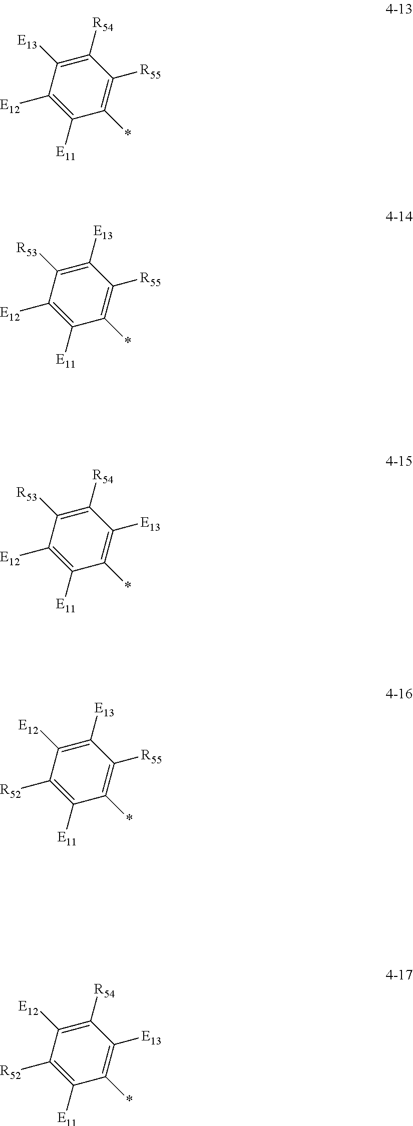

wherein, in Formulae 4-1 to 4-17,
E₁₁ to E₁₃ are each as described in connection with E₁,
R₅₁ to R₅₅ are each as described in connection with R₅, and
* indicates a binding site to a neighboring atom.

In one or more embodiments, at least one of R₅₁ to R₅₅ in Formulae 4-1 and 4-17 may be deuterium.

In one or more embodiments, some or all of R₅₁ to R₅₅ in Formula 4-1 to 4-17 may be deuterium.

In one or more embodiments, in Formulae 4-4 to 4-12, E₁₁ and E₁₂ may be identical to or different from each other.

In one or more embodiments, in Formulae 4-13 to 4-17, E₁₁ and E₁₃ may be identical to or different from each other.

In one or more embodiments, a moiety represented by

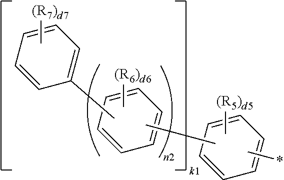

in Formula 1A may be a group represented by one of Formulae 5-1 to 5-15:

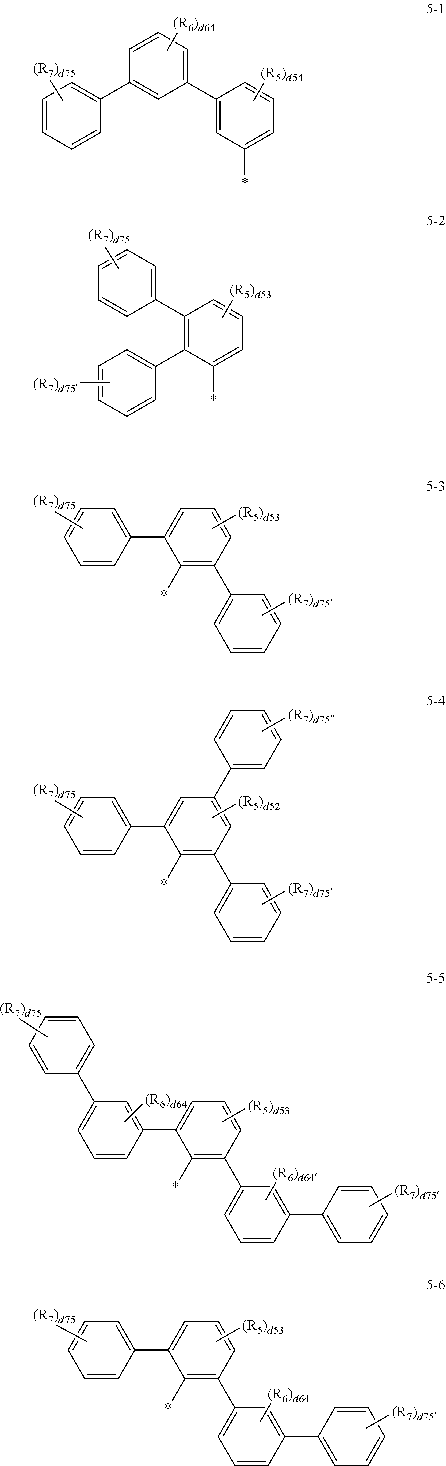

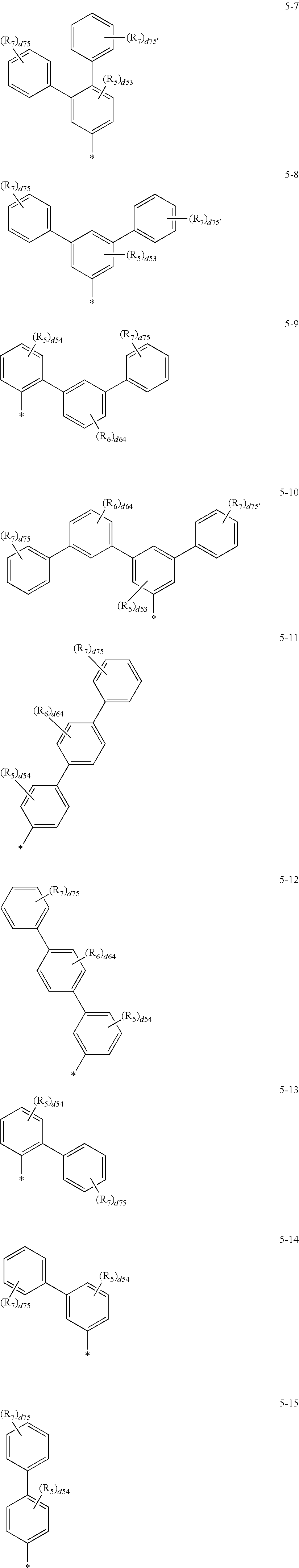

wherein, in Formulae 5-1 to 5-15,
R₅ to R₇ are each as described herein,
d75, d75′, and d75″ may each independently be an integer from 0 to 5,
d54, d64, and d64′ may each independently be an integer from 0 to 4,
d53 may be an integer from 0 to 3, and
d52 may be an integer from 0 to 2.

In one or more embodiments, when n1 in Formula 1A is 2, a moiety represented by

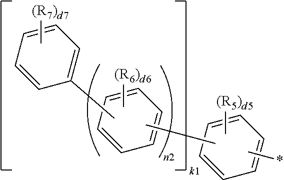

in Formula 1A may be a group represented by one of Formulae 5-10 to 5-15.

In one or more embodiments, when n1 in Formula 1A is 3, a moiety represented by

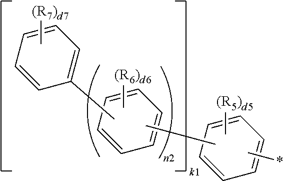

in Formula 1A may be a group represented by one of Formulae 5-1 to 5-15.

The heterocyclic compound represented by Formula 1 may be represented by one of Formulae 1-1 to 1-36, 1-73 to 1-111, 1-145 to 1-180, or 1-217 to 1-252:

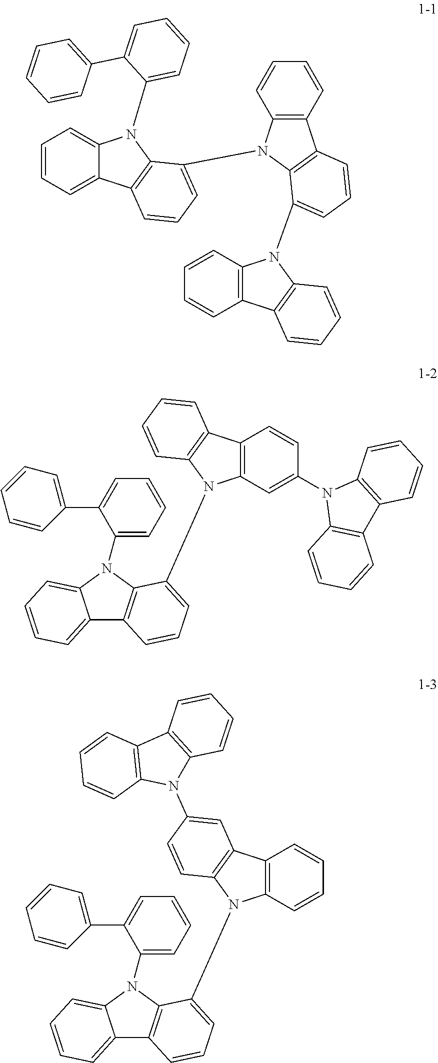

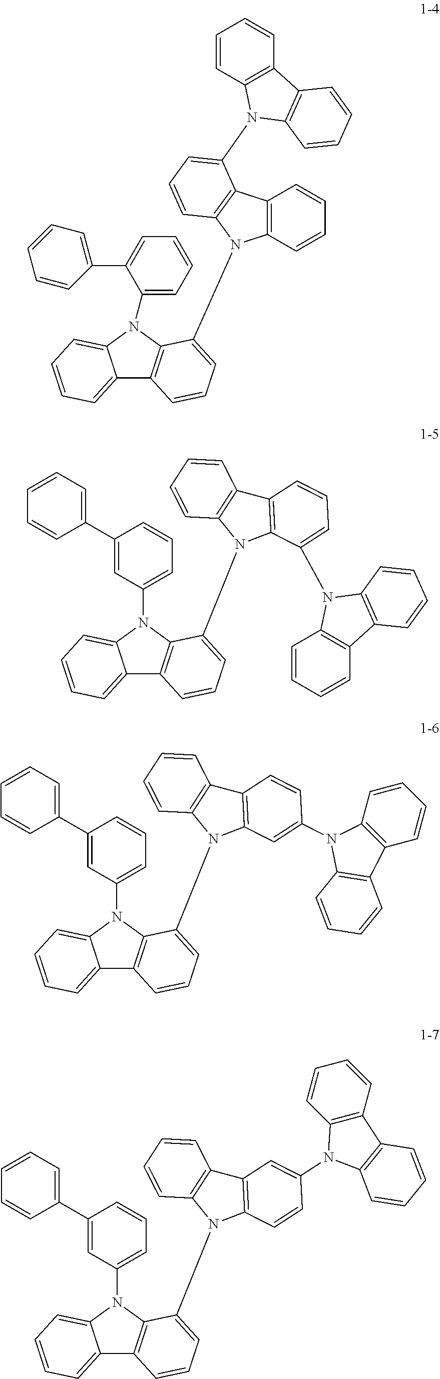

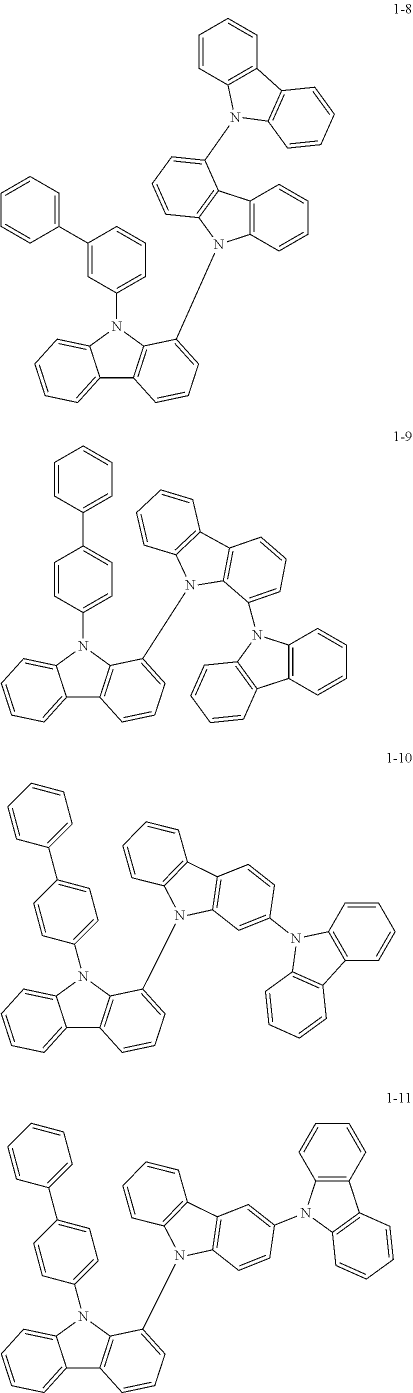

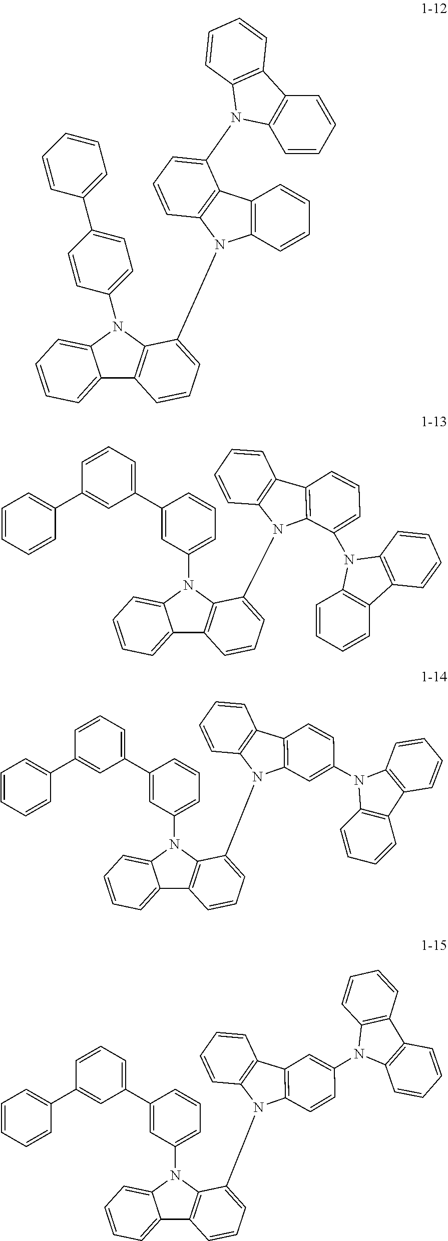

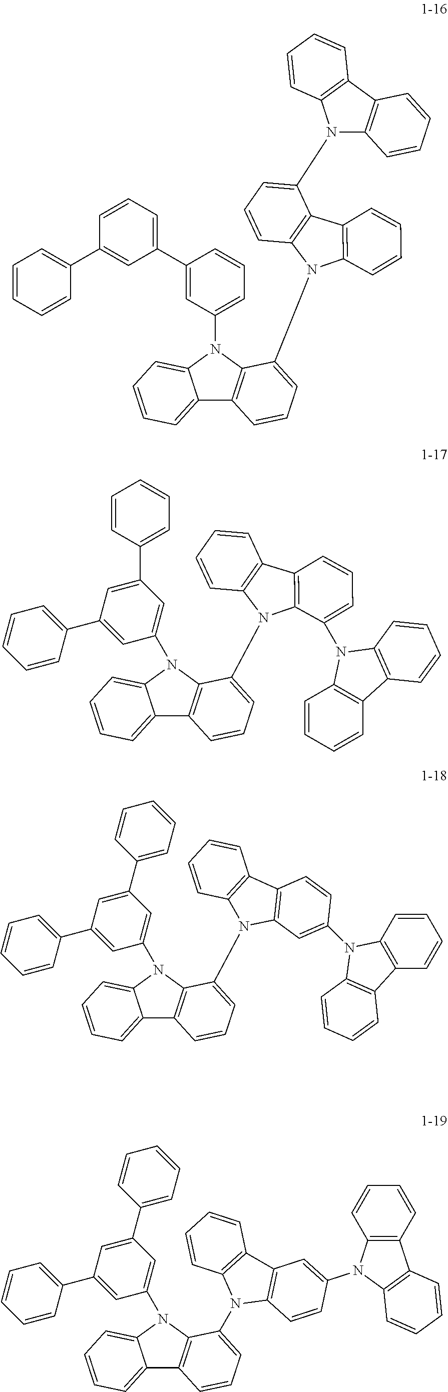

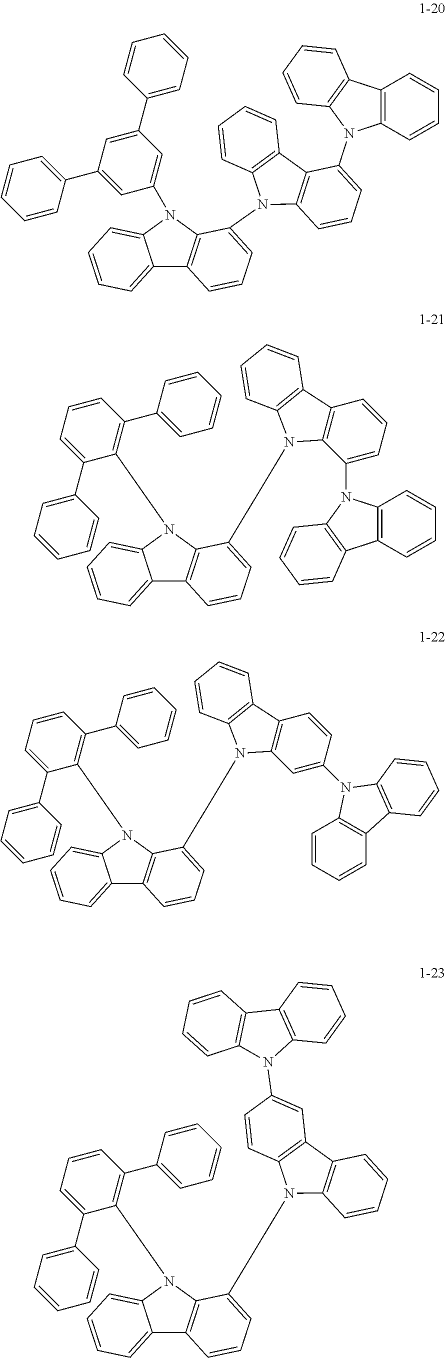

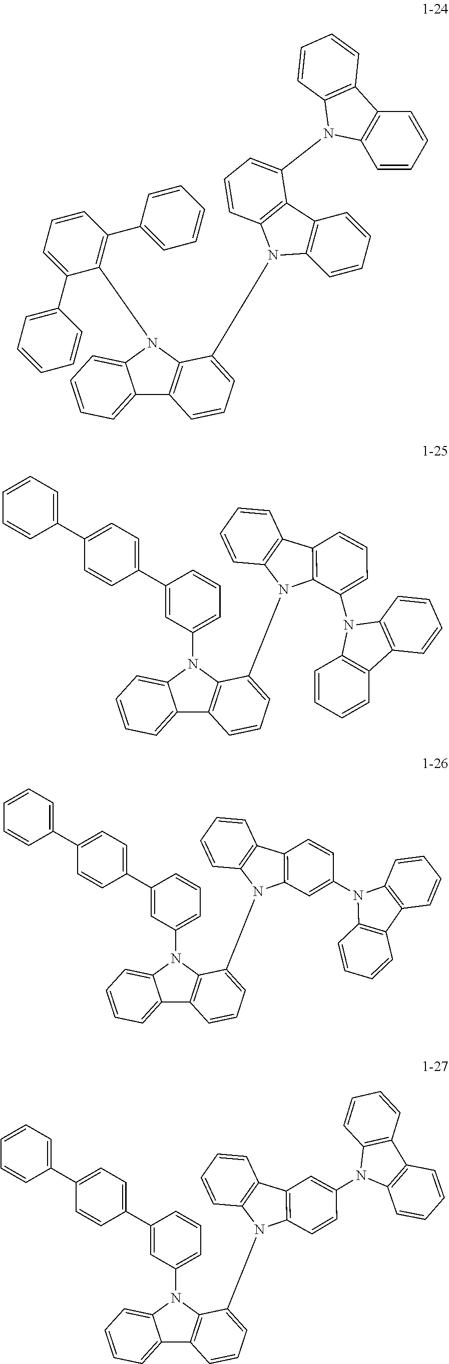

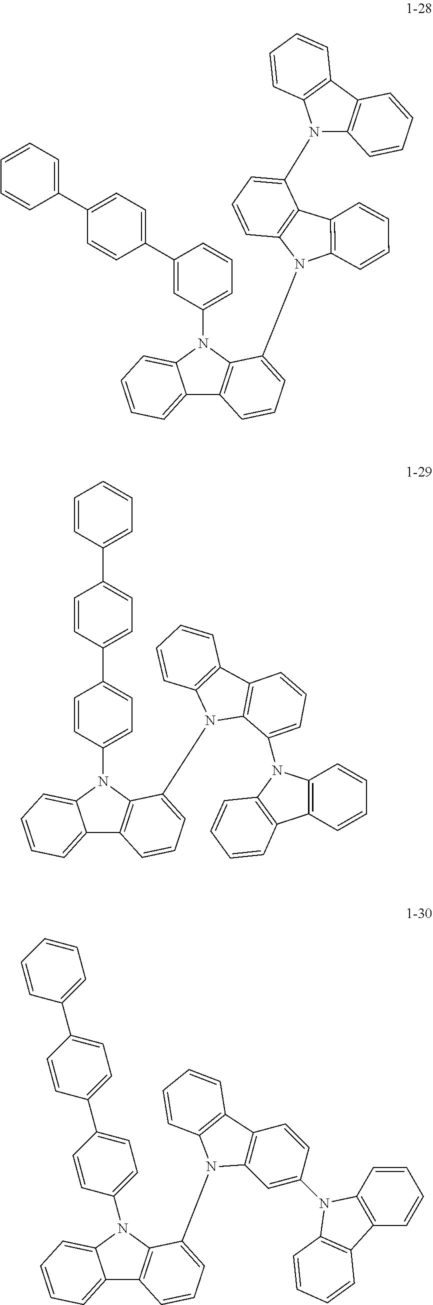

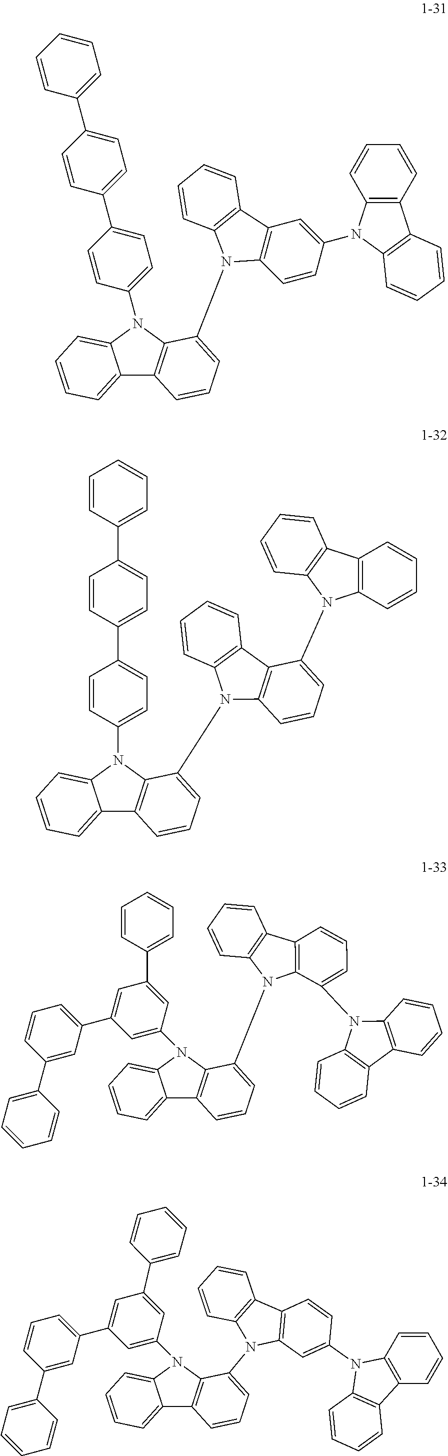

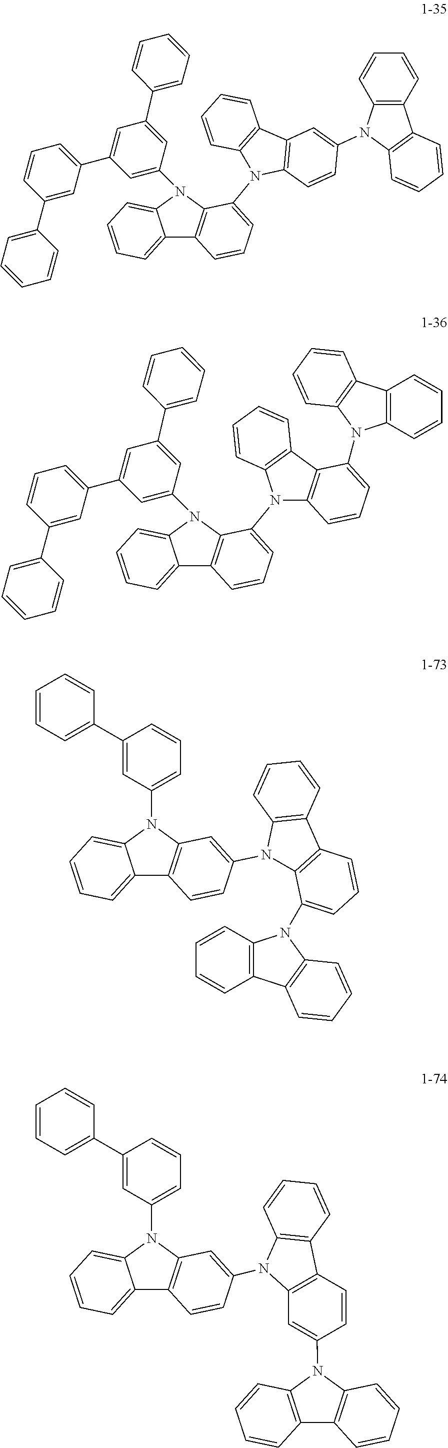

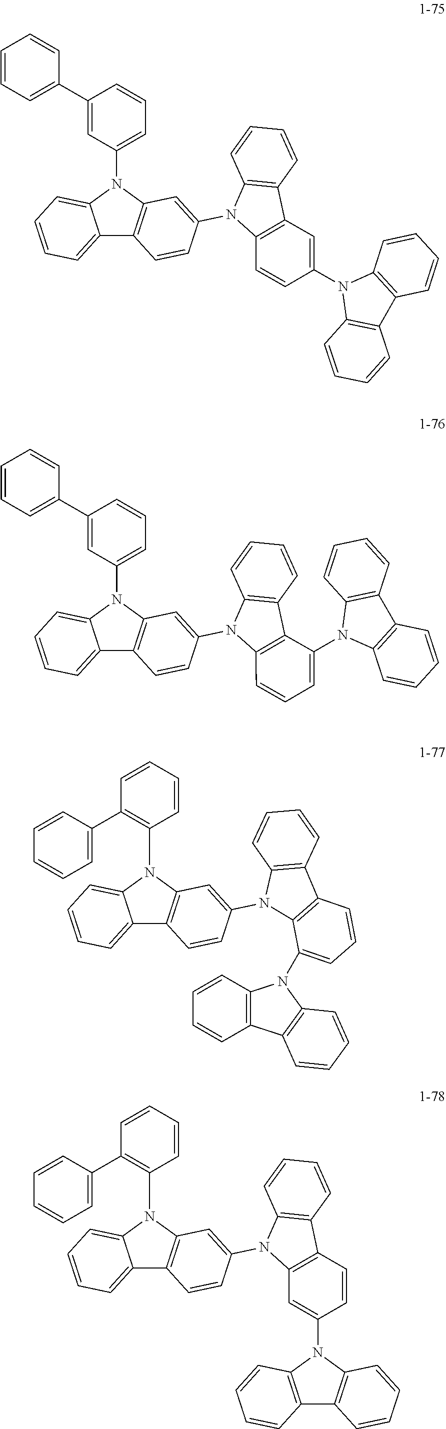

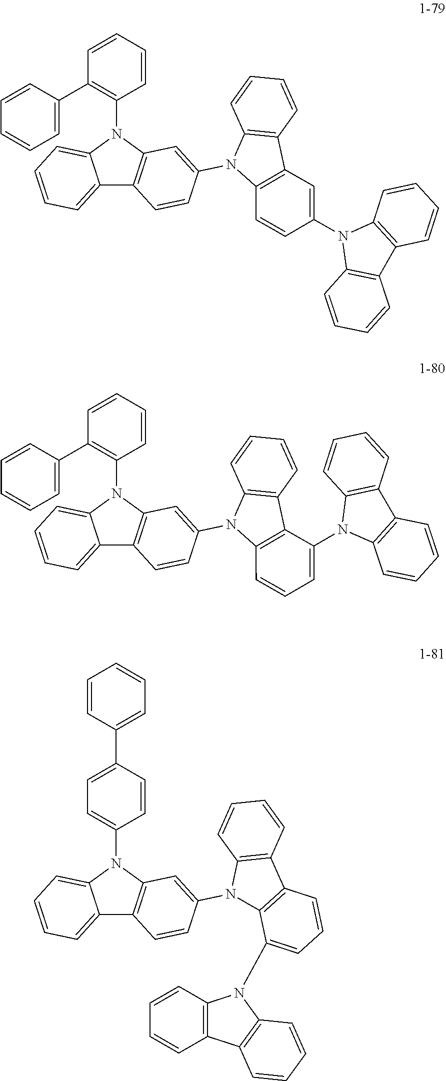

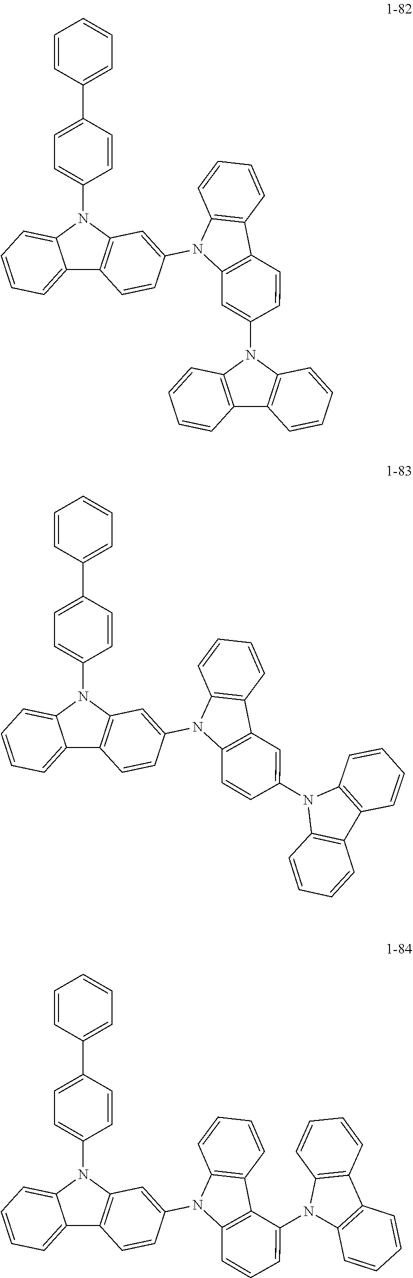

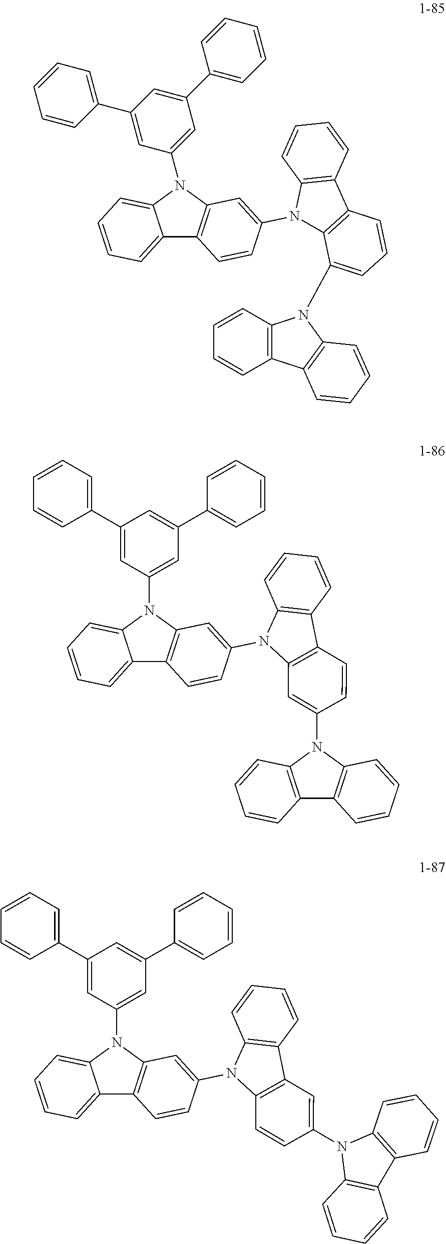

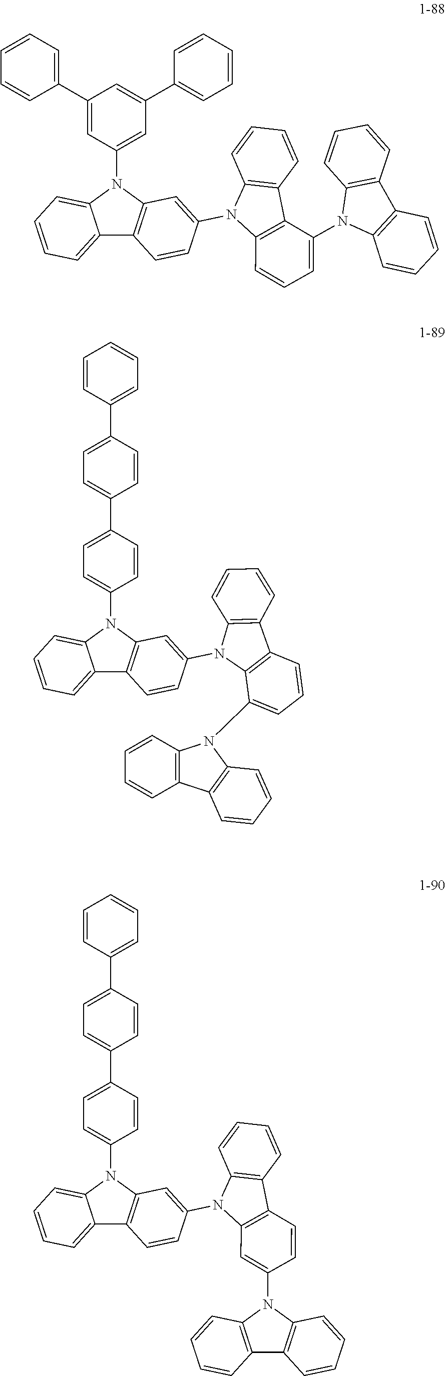

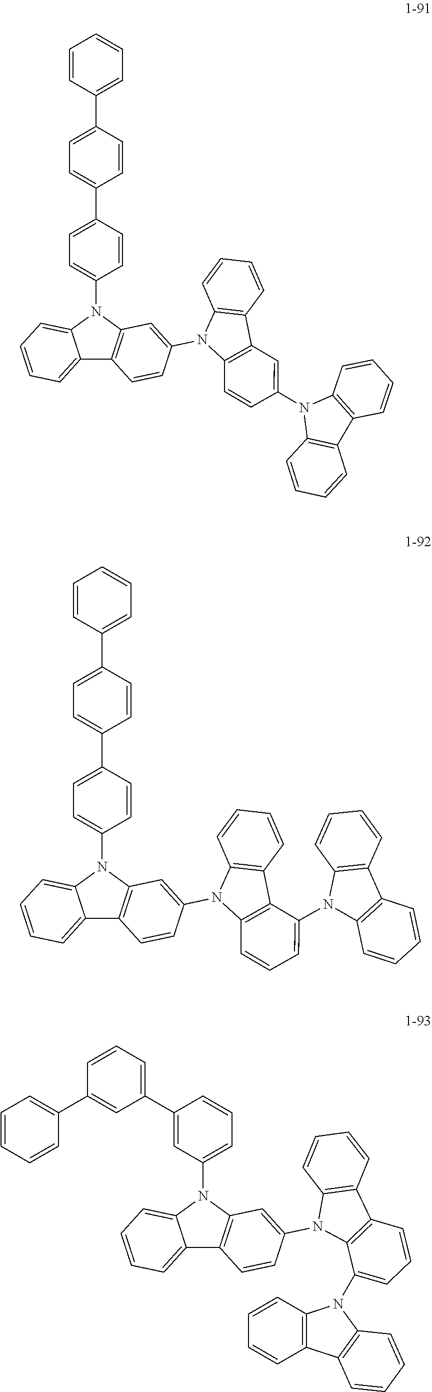

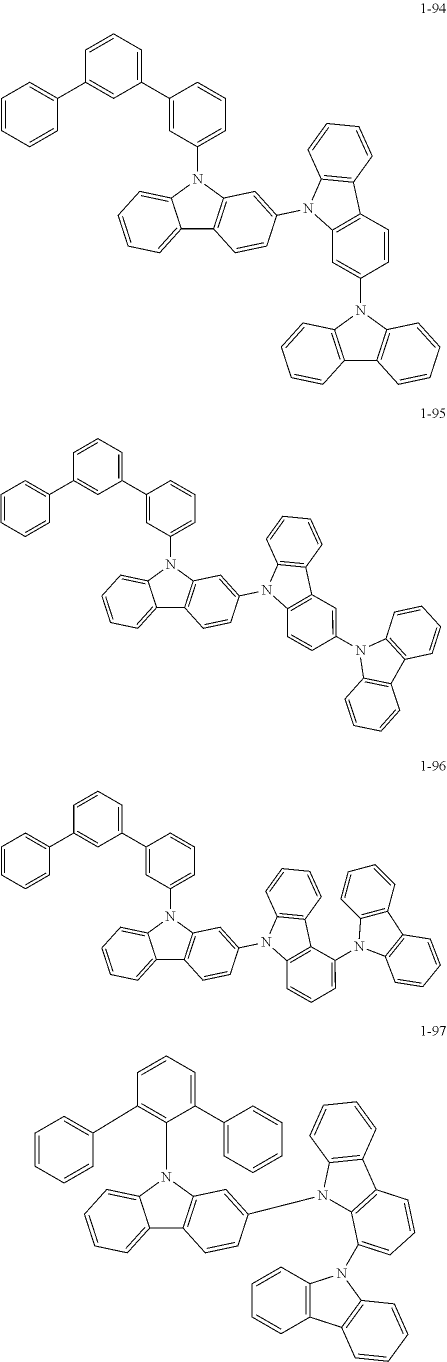

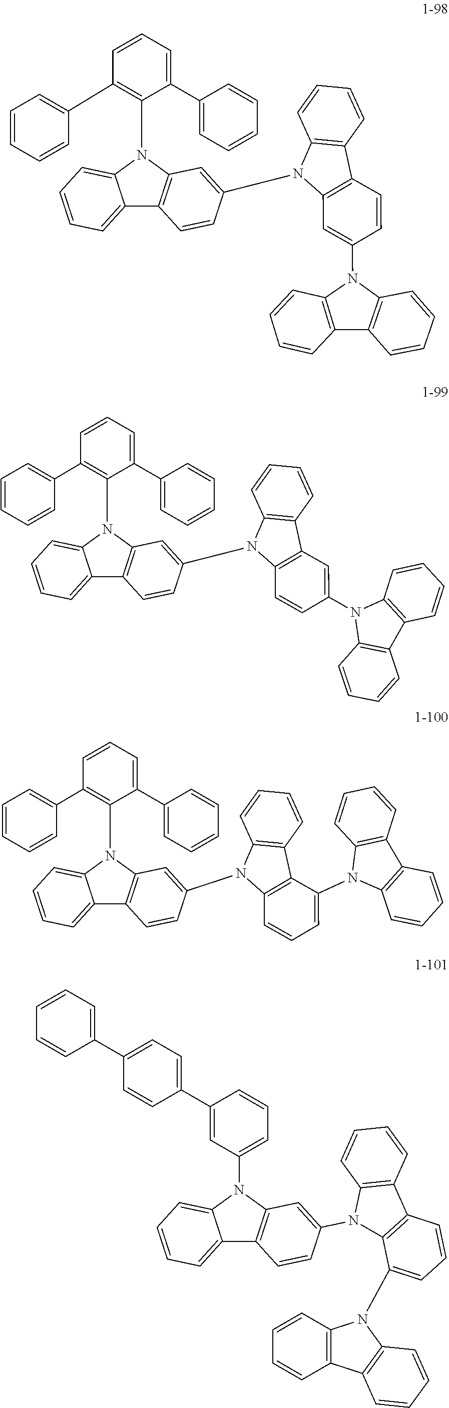

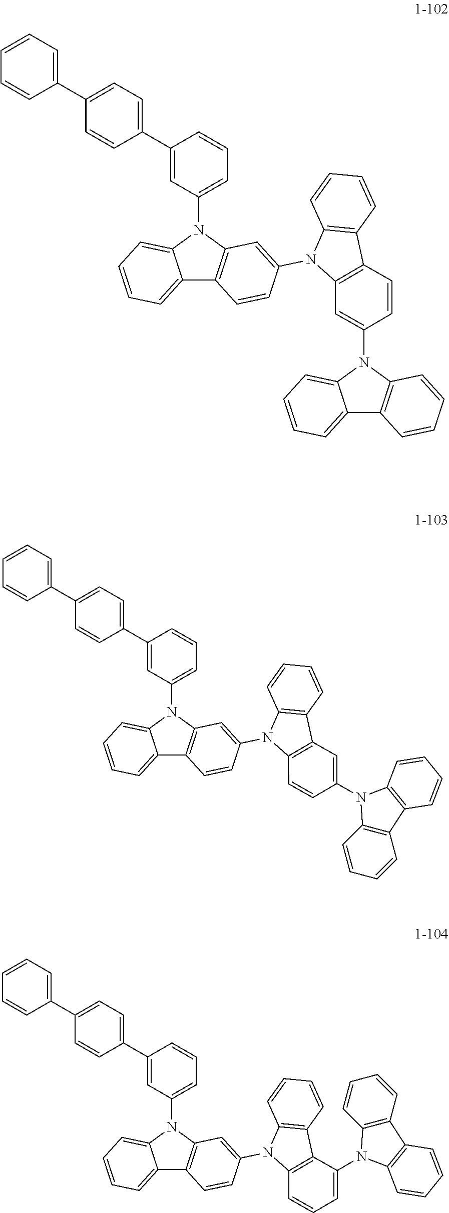

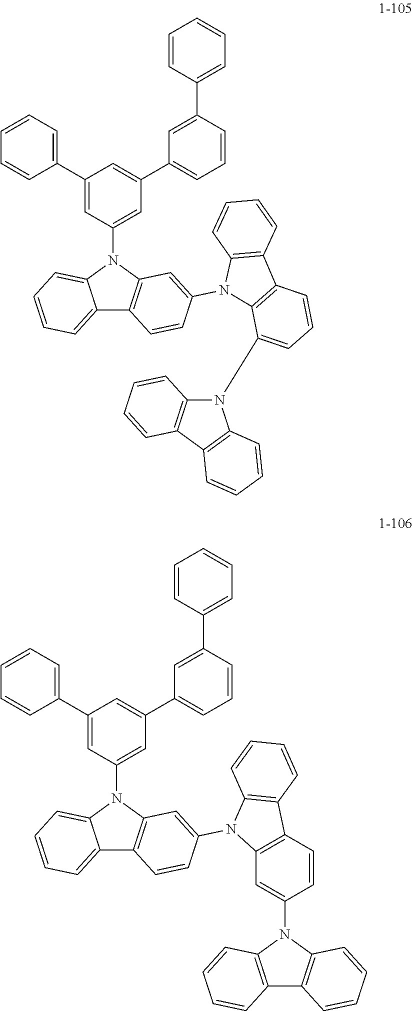

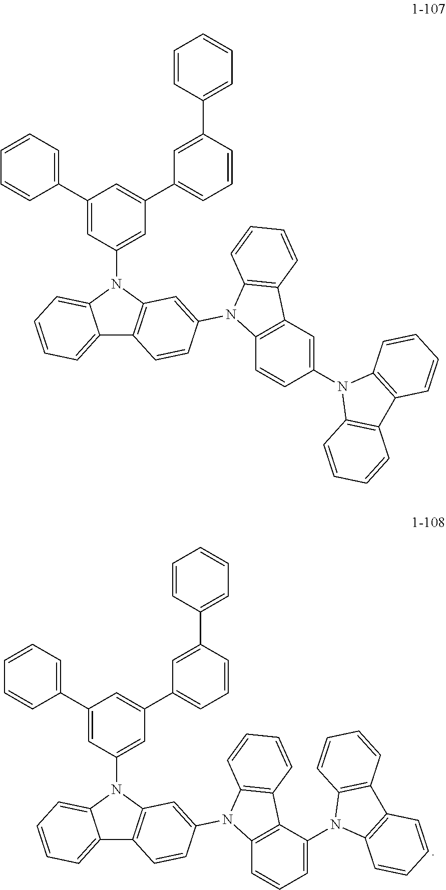

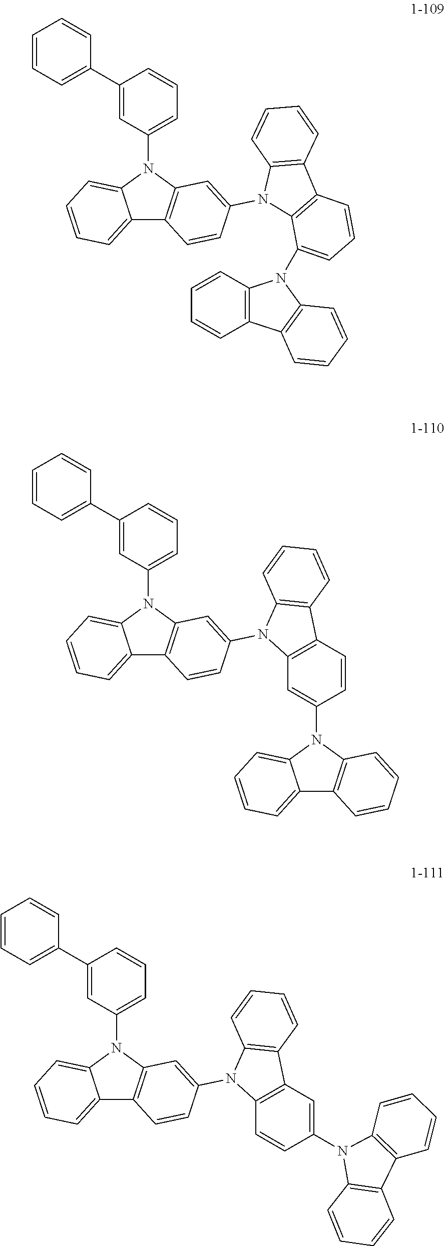

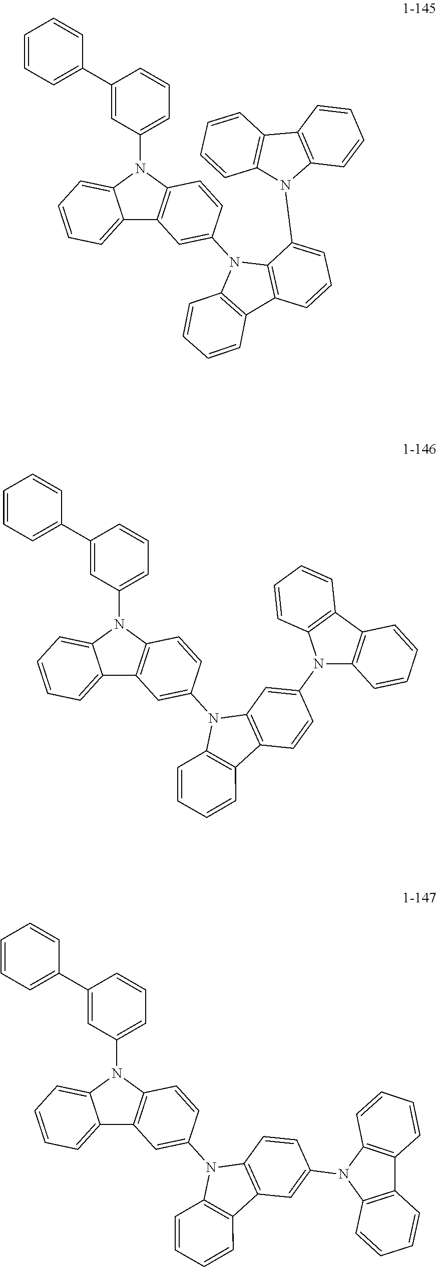

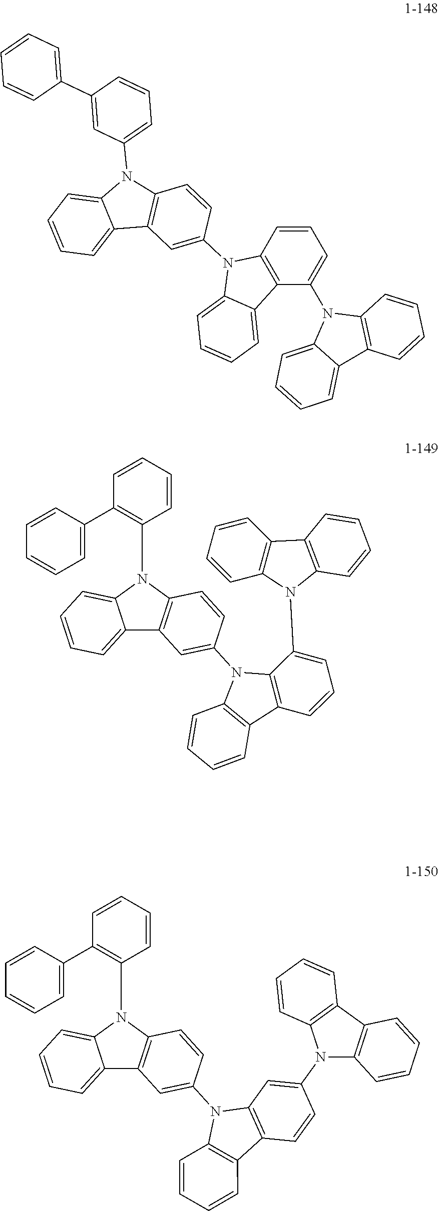

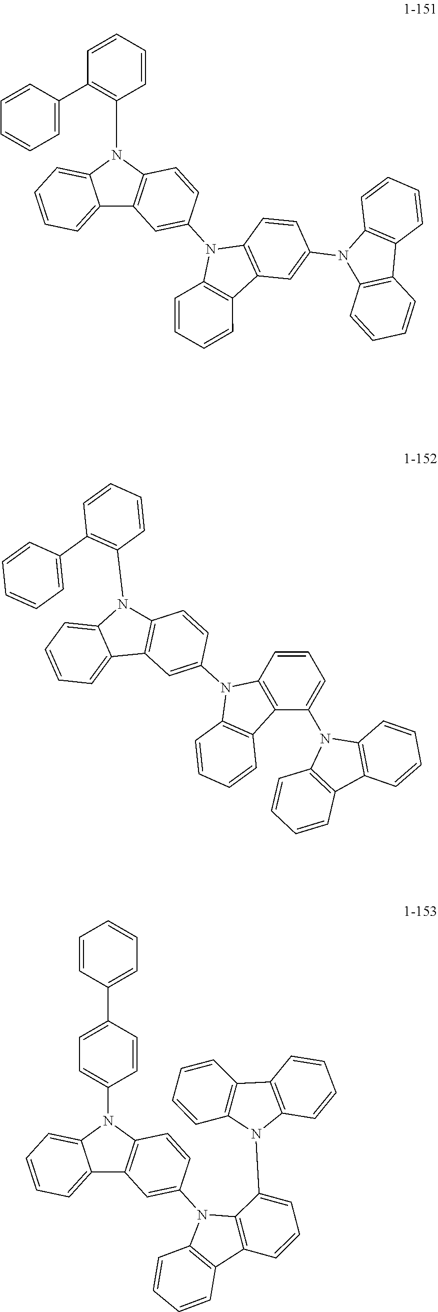

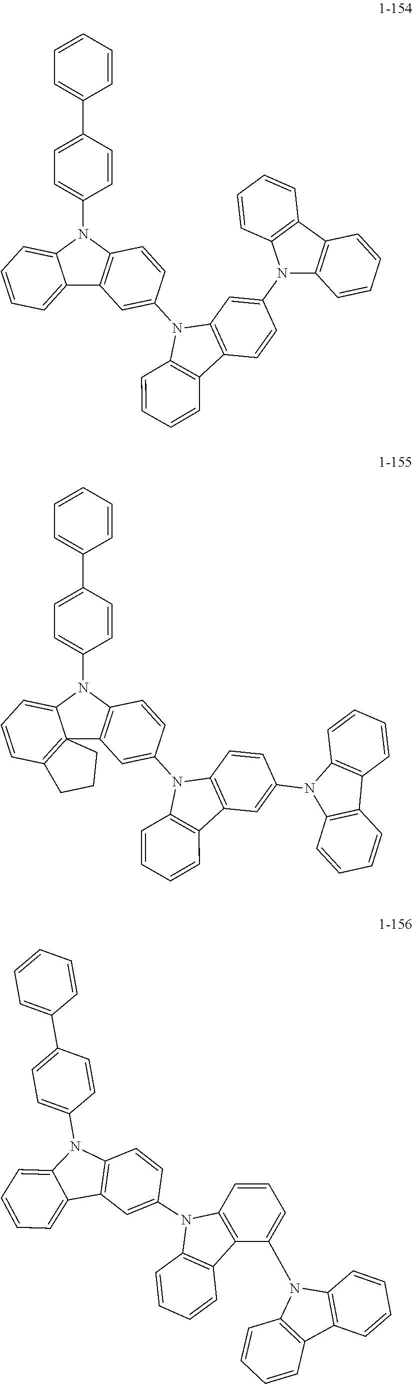

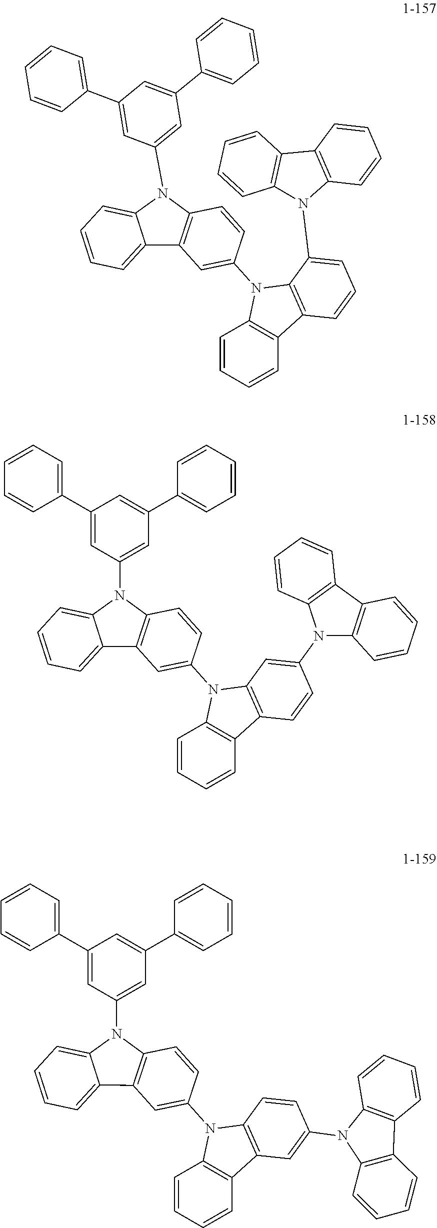

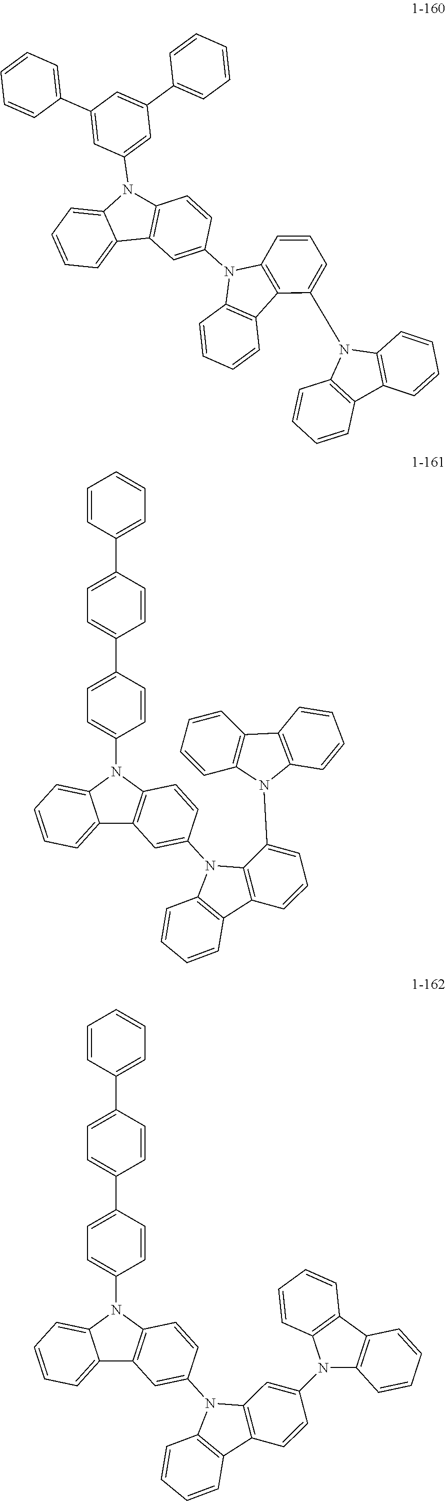

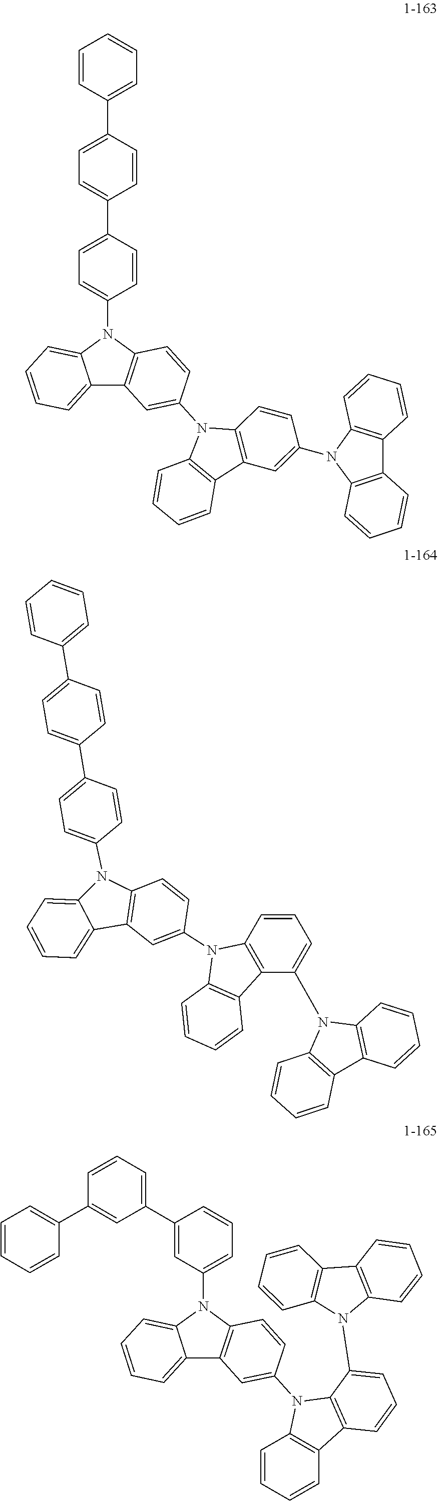

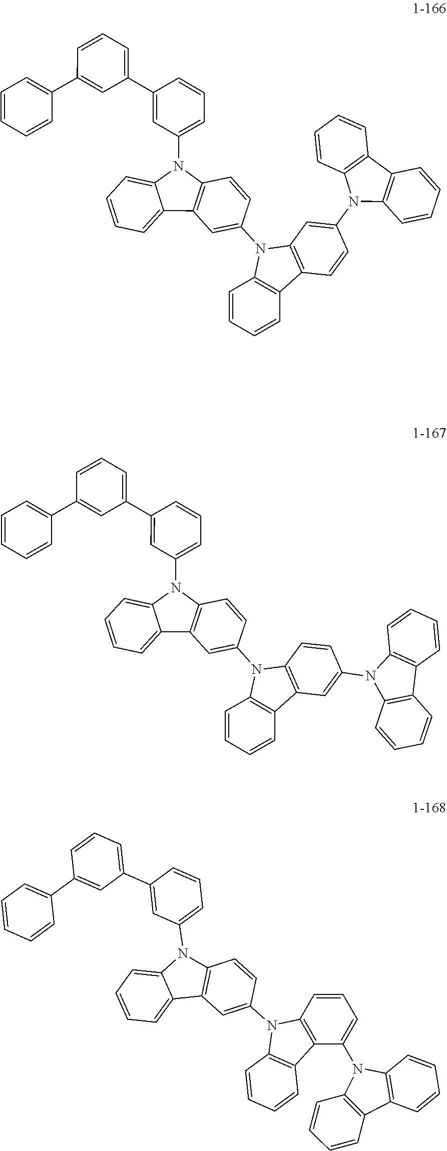

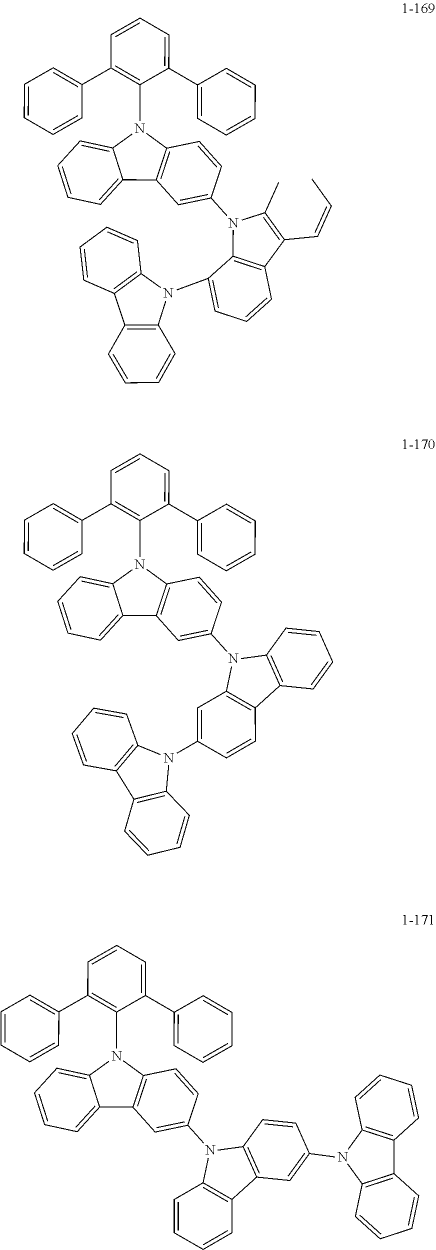

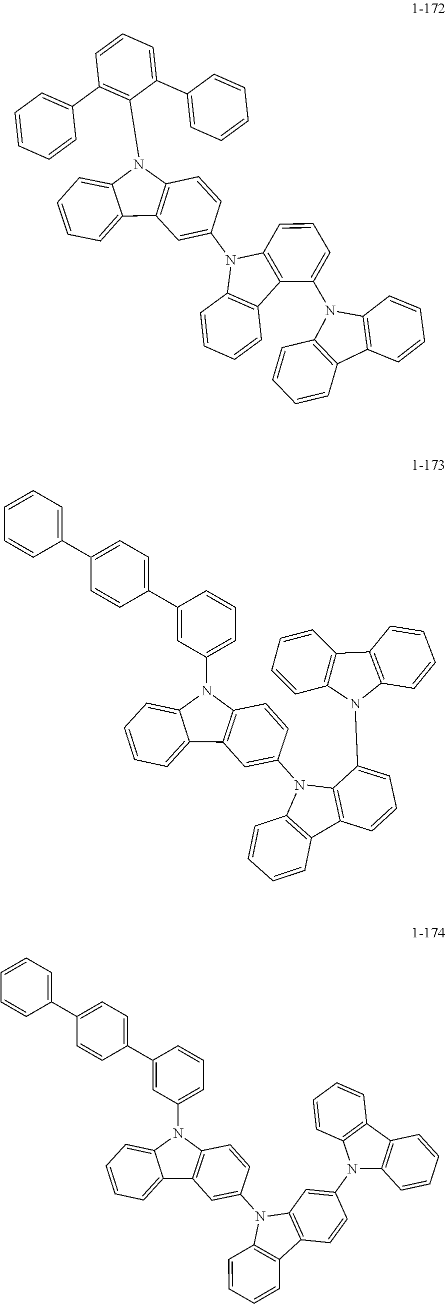

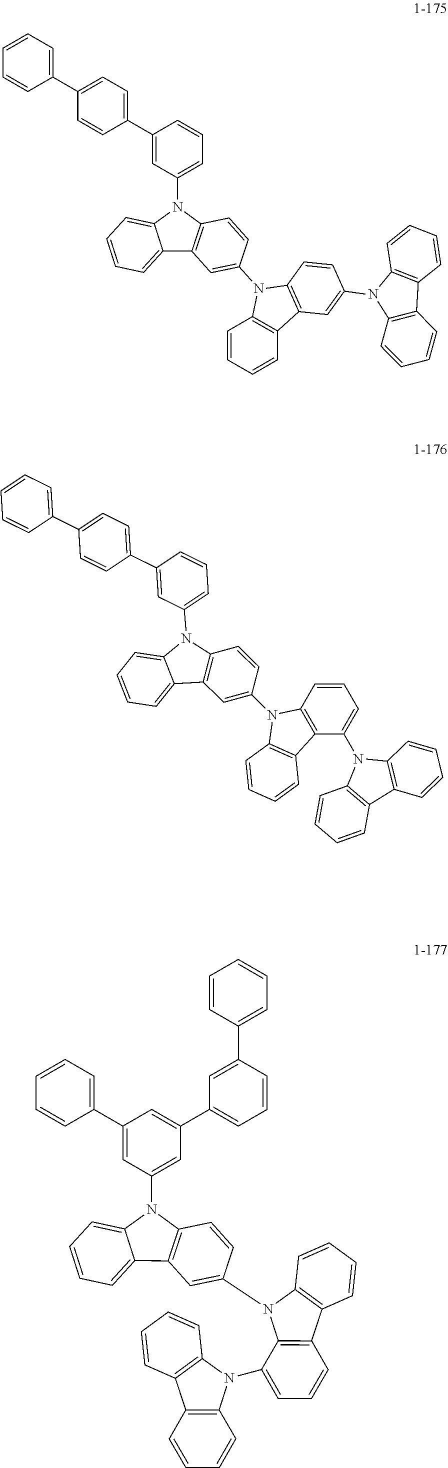

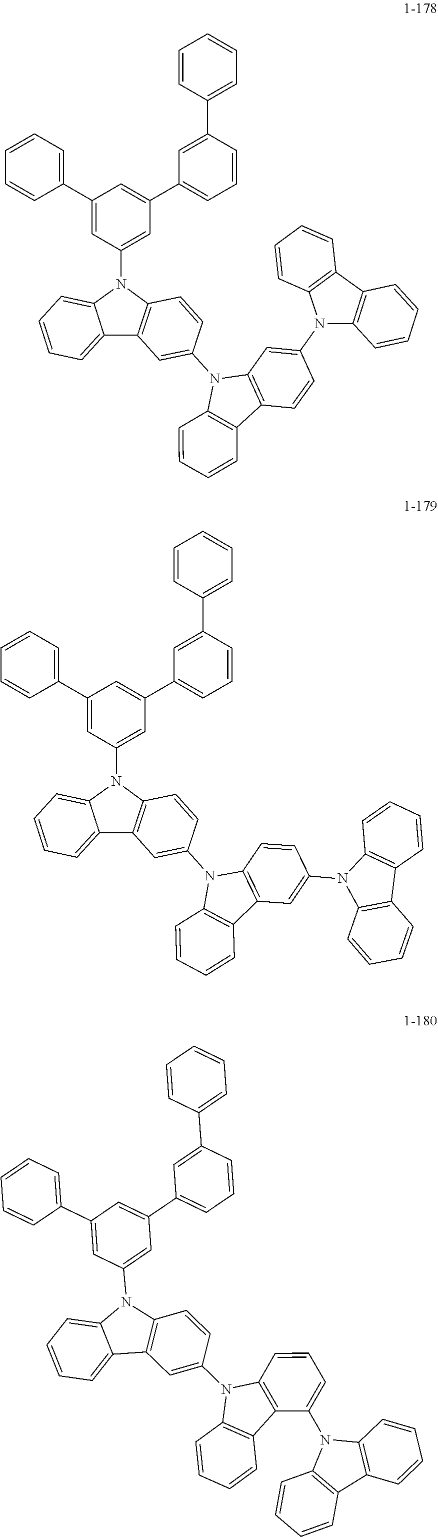

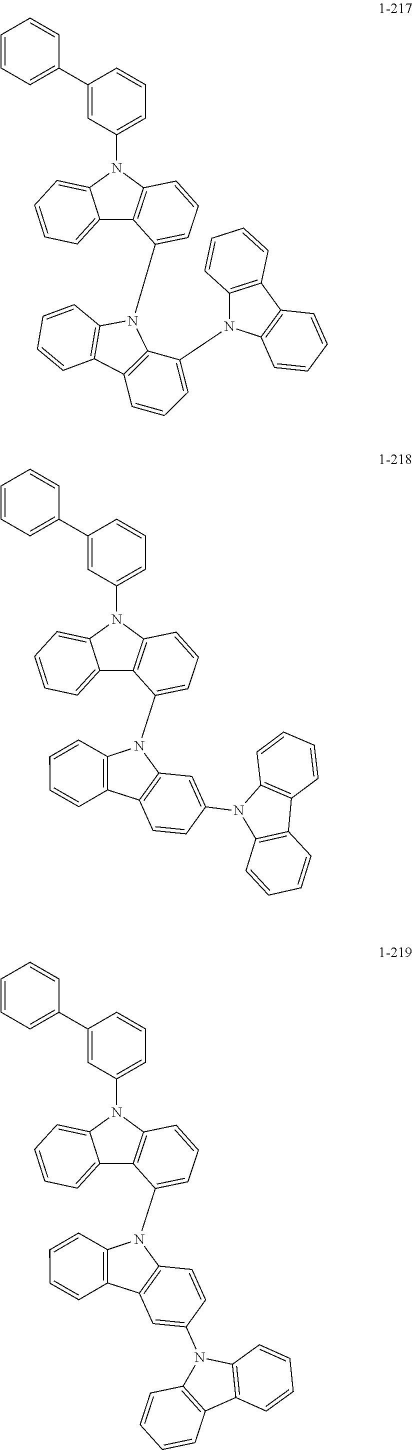

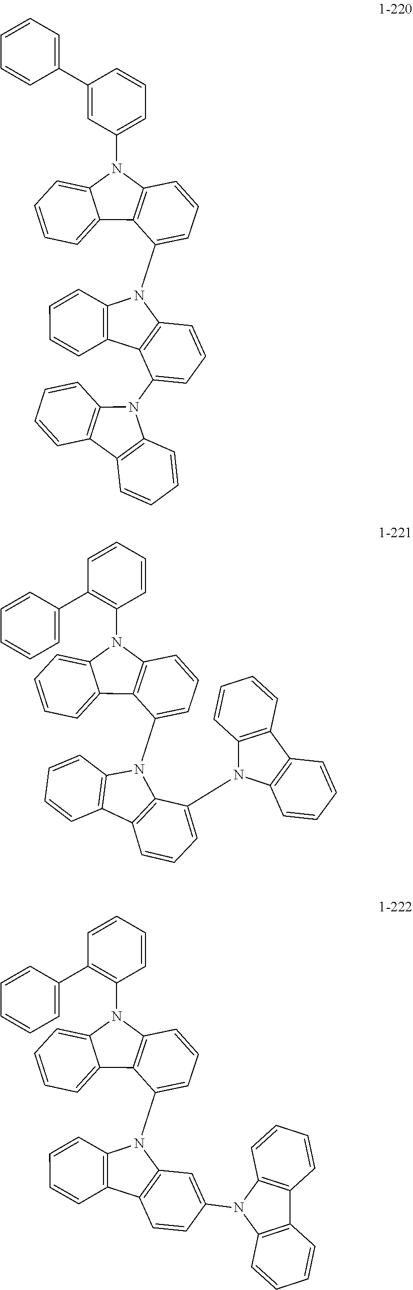

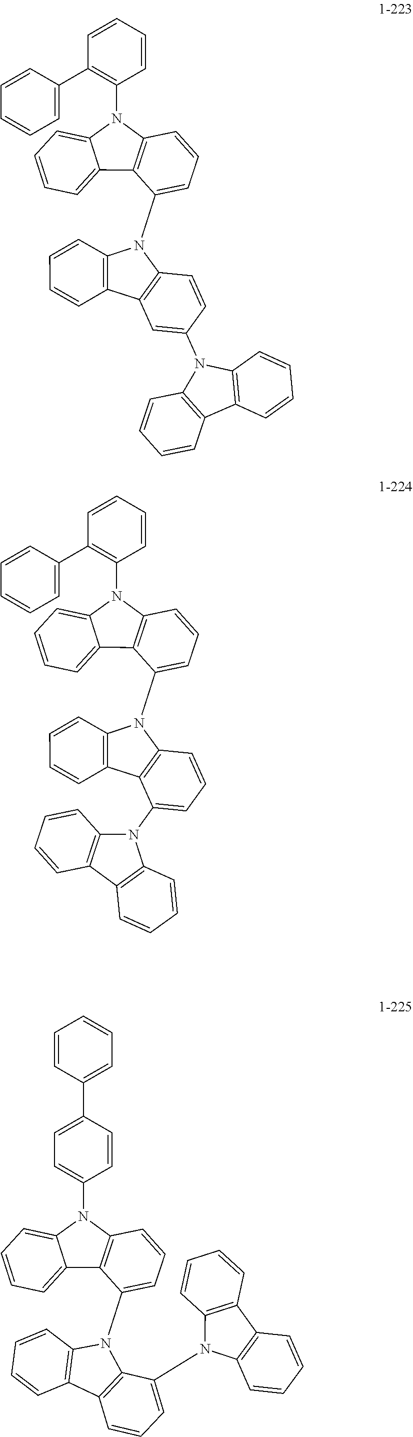

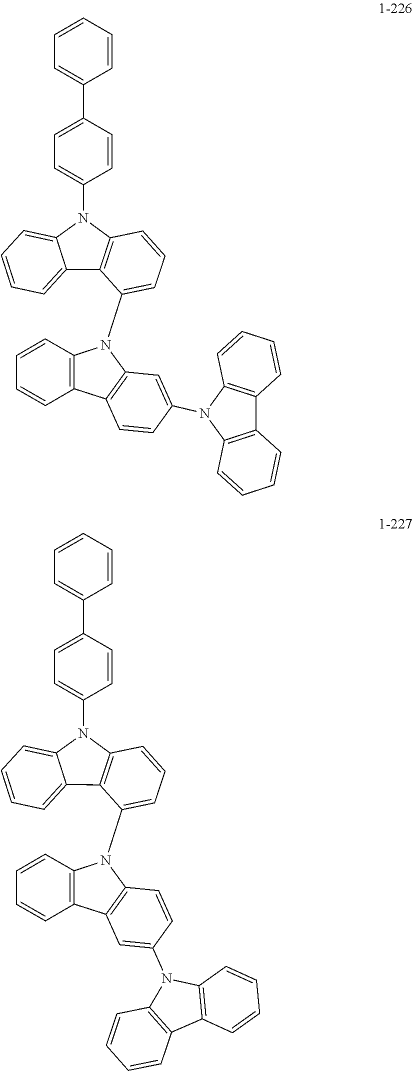

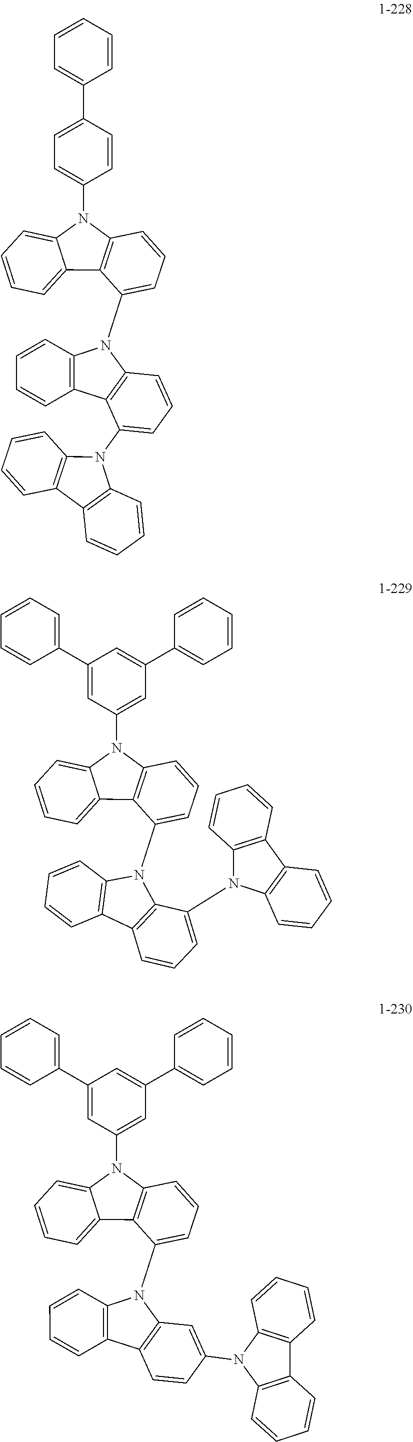

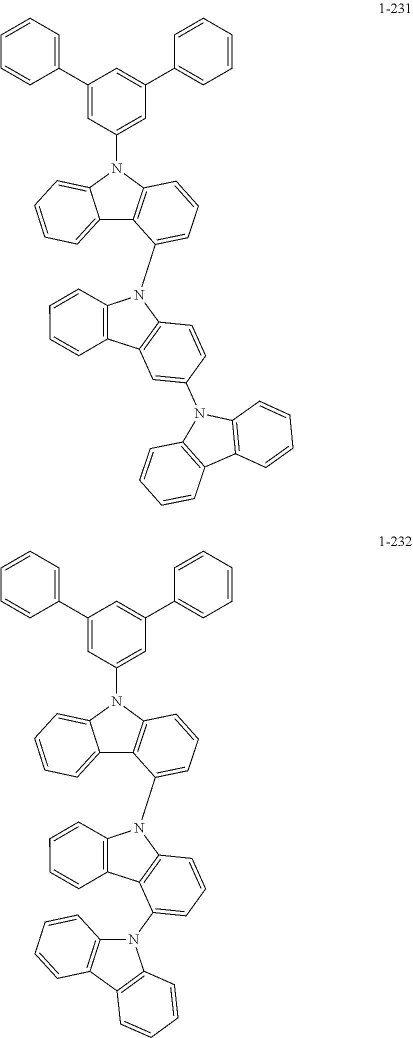

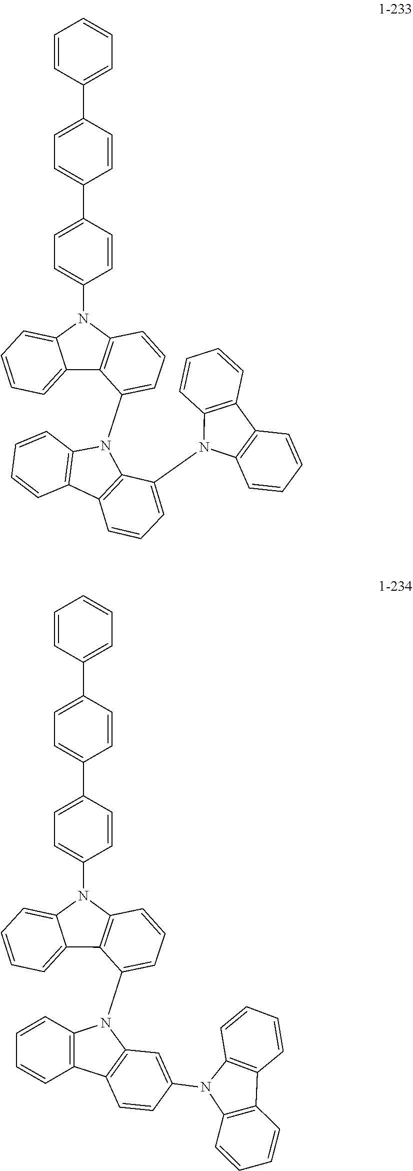

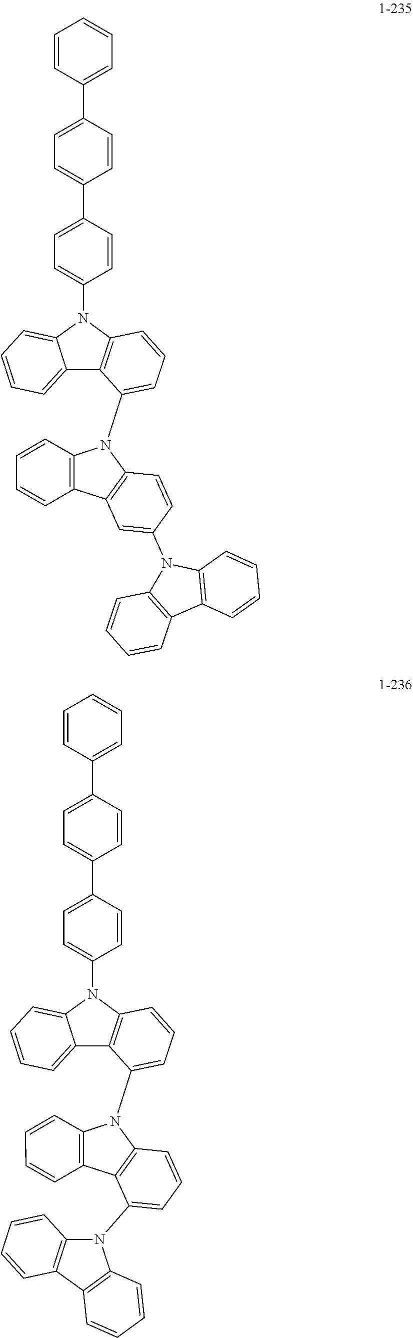

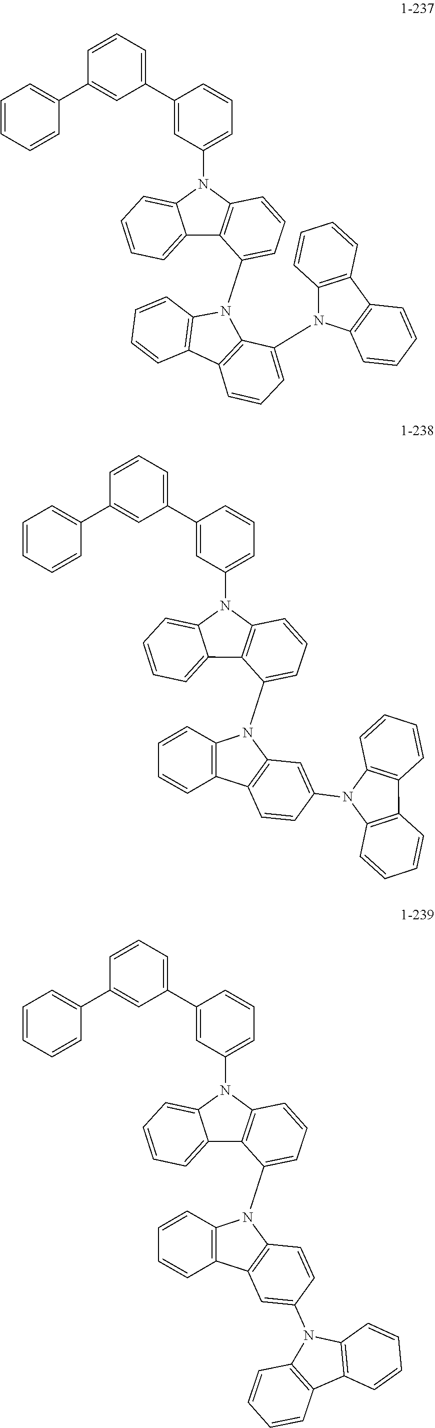

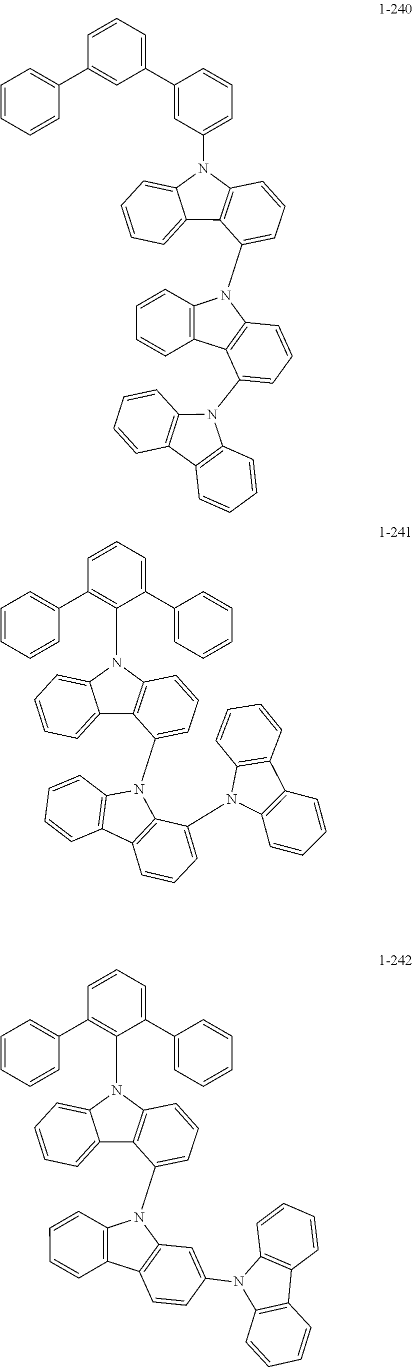

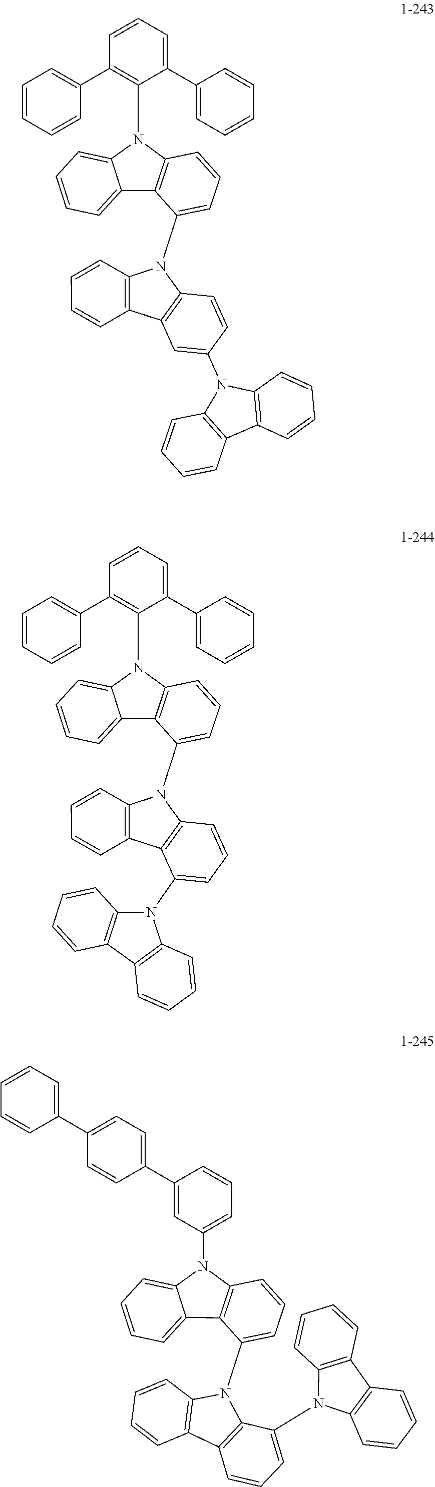

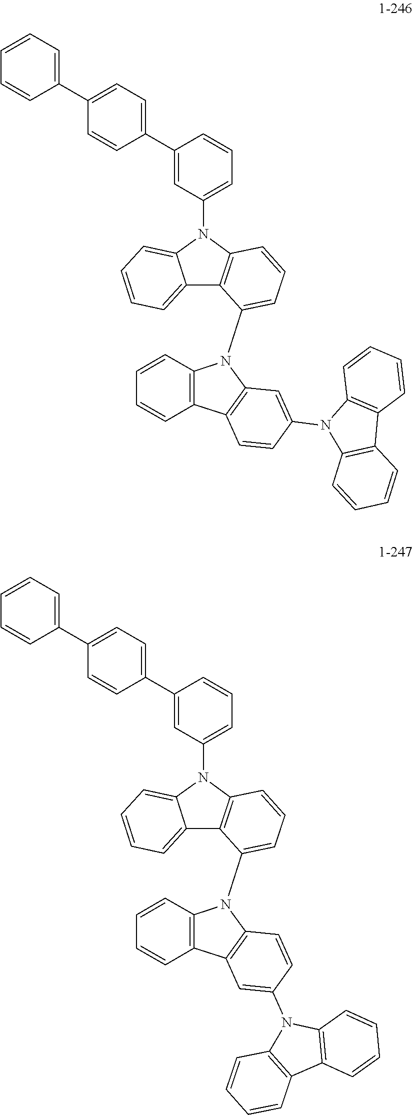

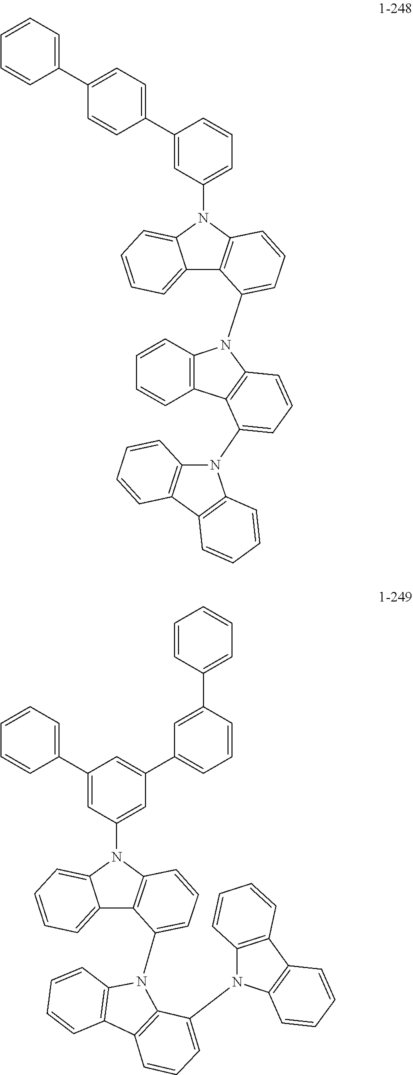

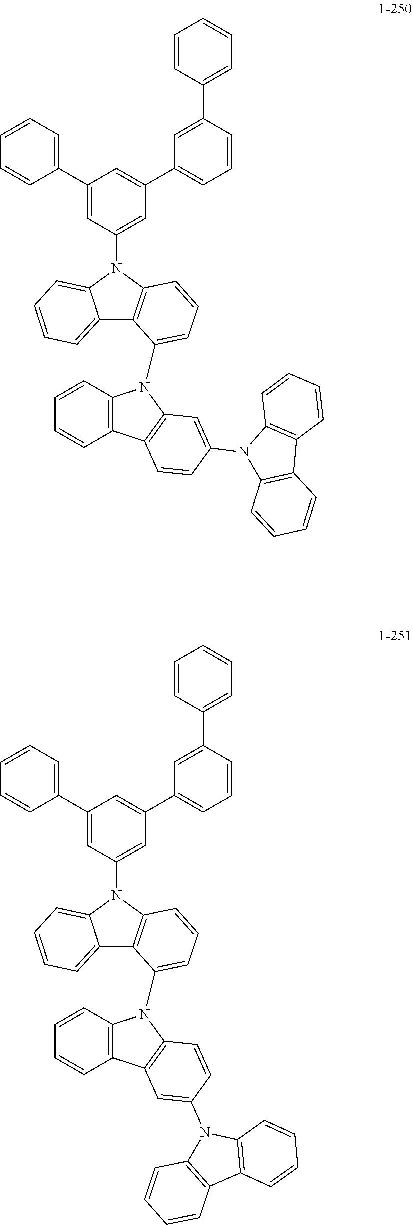

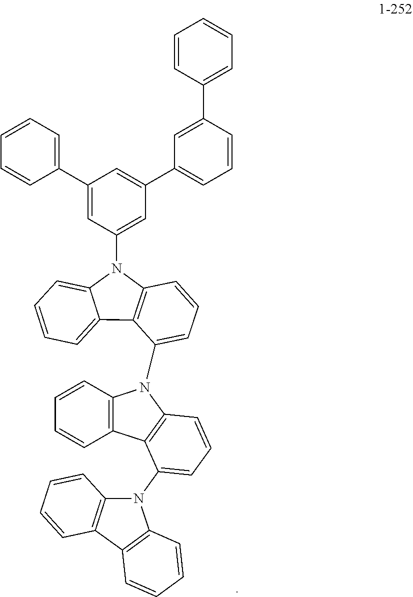

wherein, in Formulae 1-1 to 1-36, 1-73 to 1-111, 1-145 to 1-180, and 1-217 to 1-252, at least one hydrogen is substituted with deuterium.

In one or more embodiments, in Formulae 1-1 to 1-36, 1-73 to 1-111, 1-145 to 1-180, and 1-217 to 1-252, all hydrogen atoms except for those substituted with deuterium may be substituted with a substituted Rx, wherein Rx is as described in connection with R₁.

In one or more embodiments, the heterocyclic compound represented by Formula 1 may be one of Compounds 1 to 580:

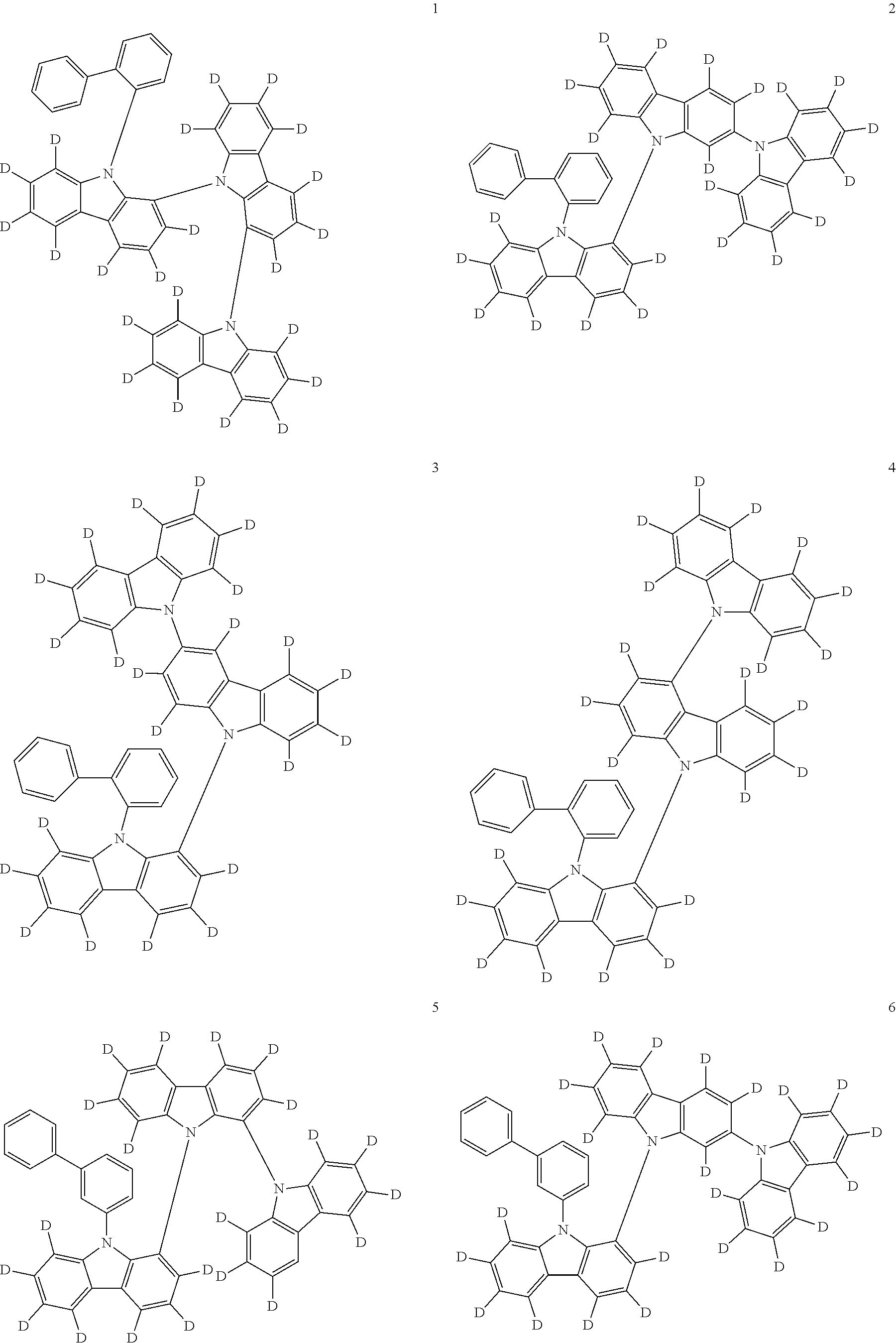

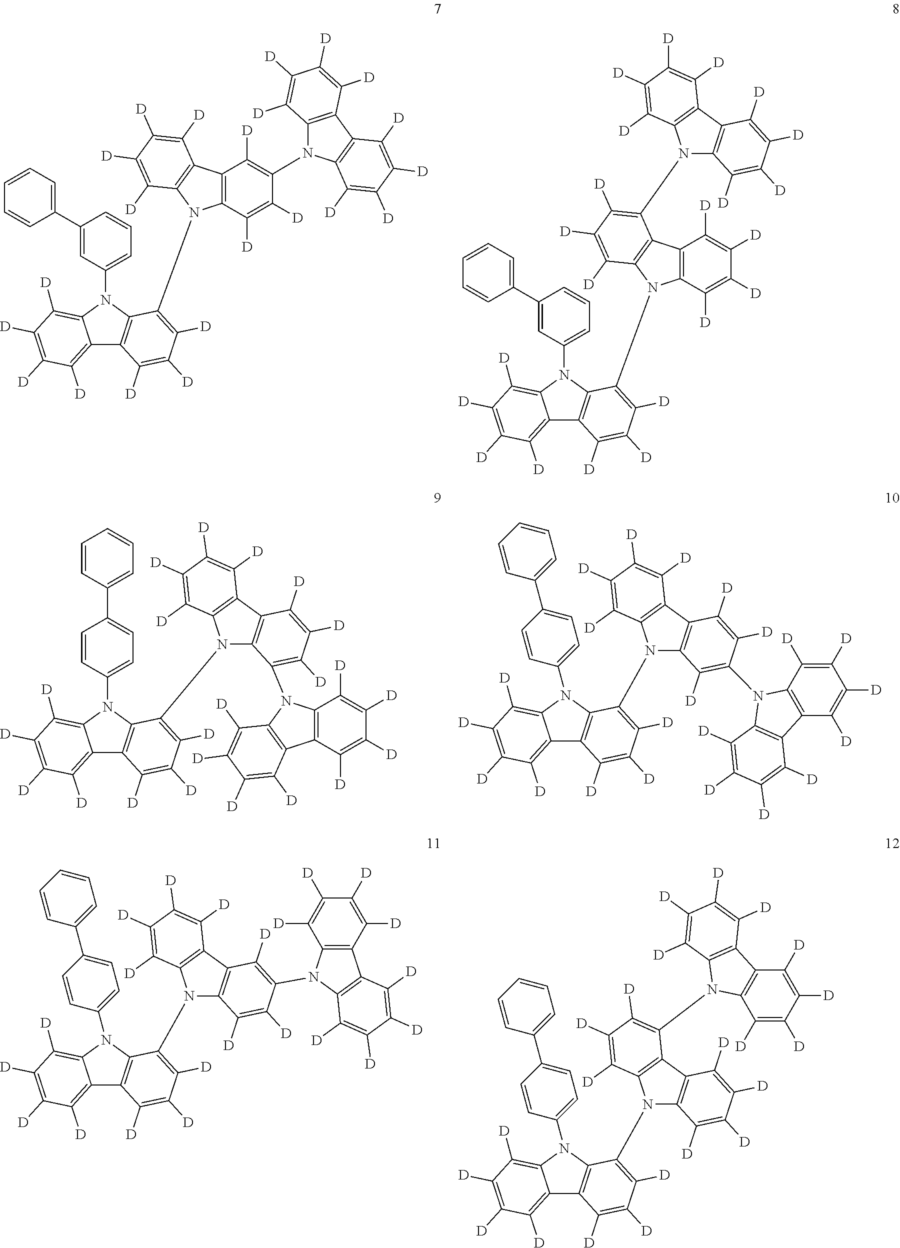

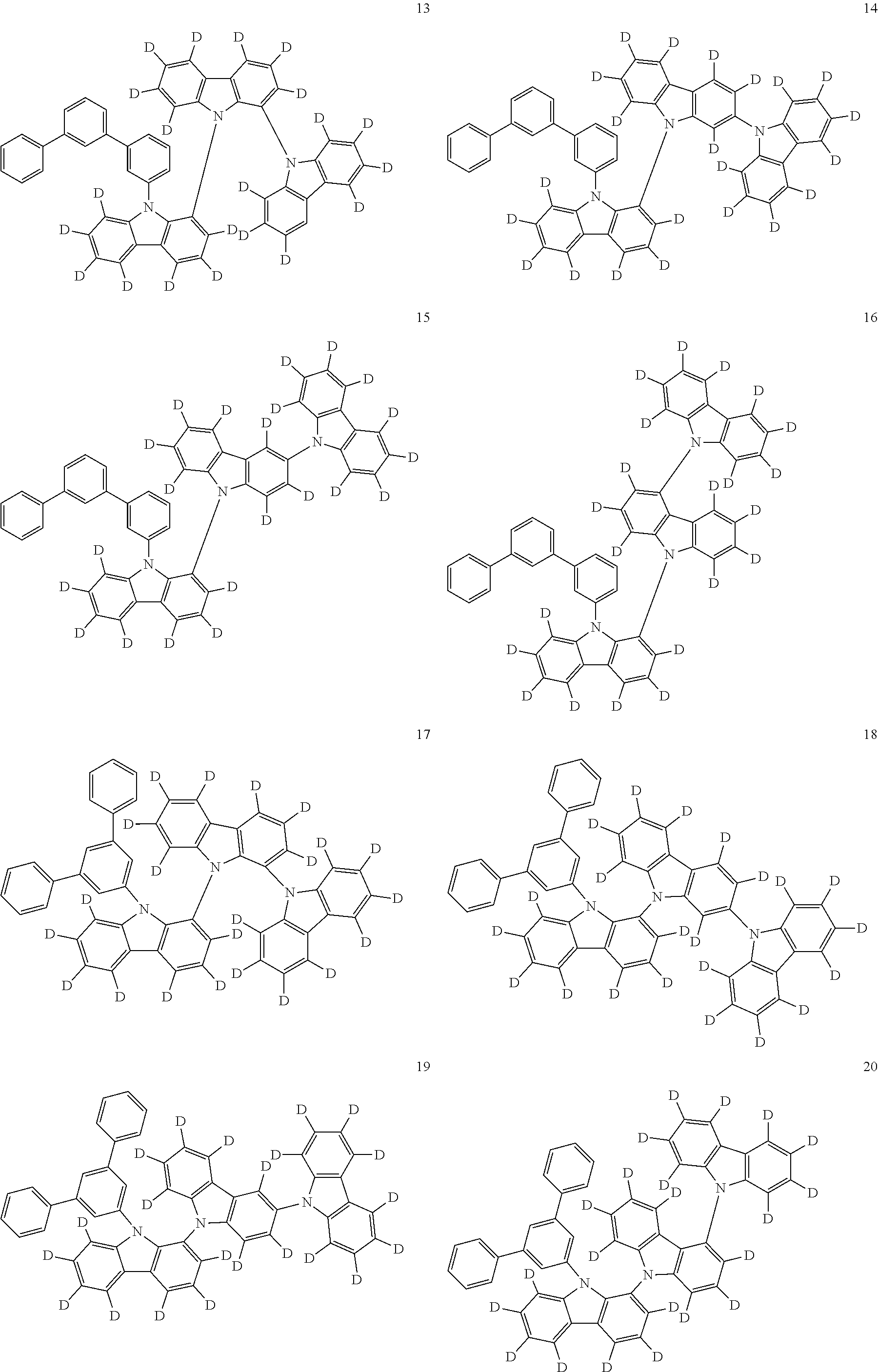

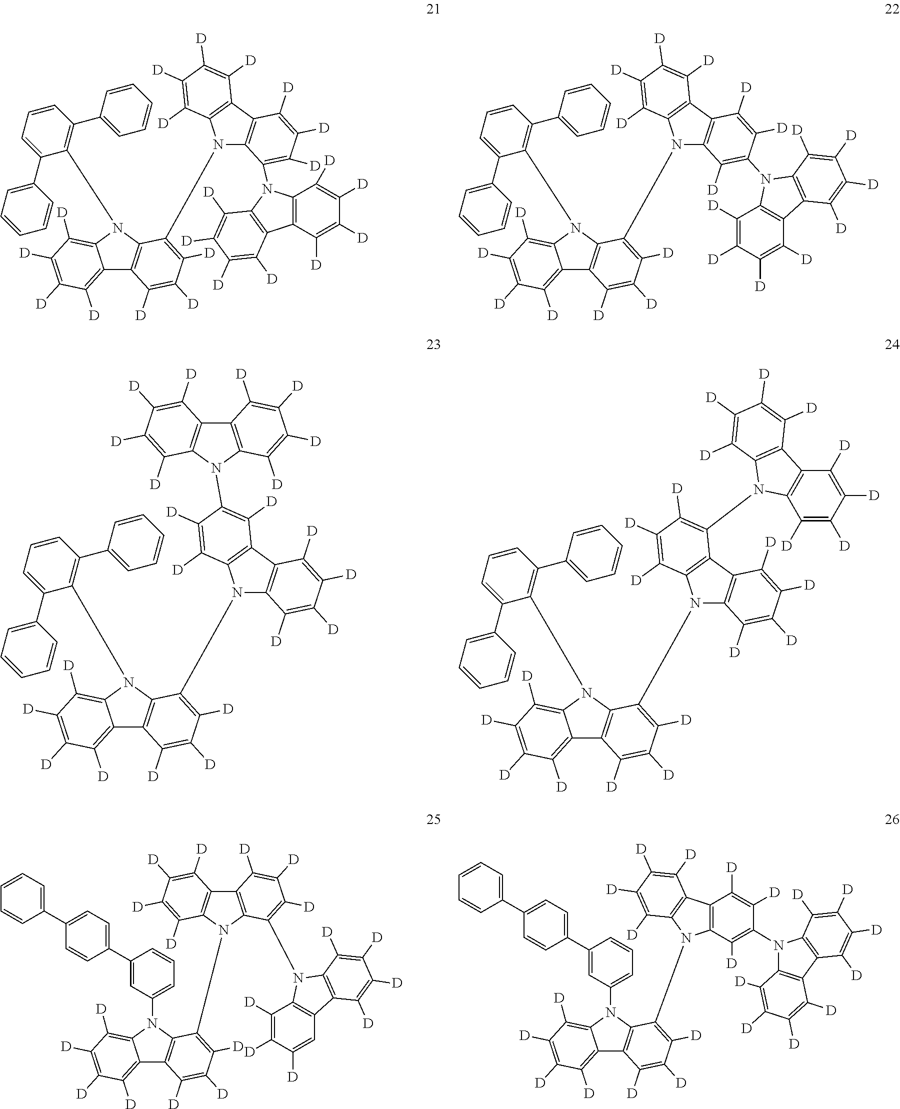

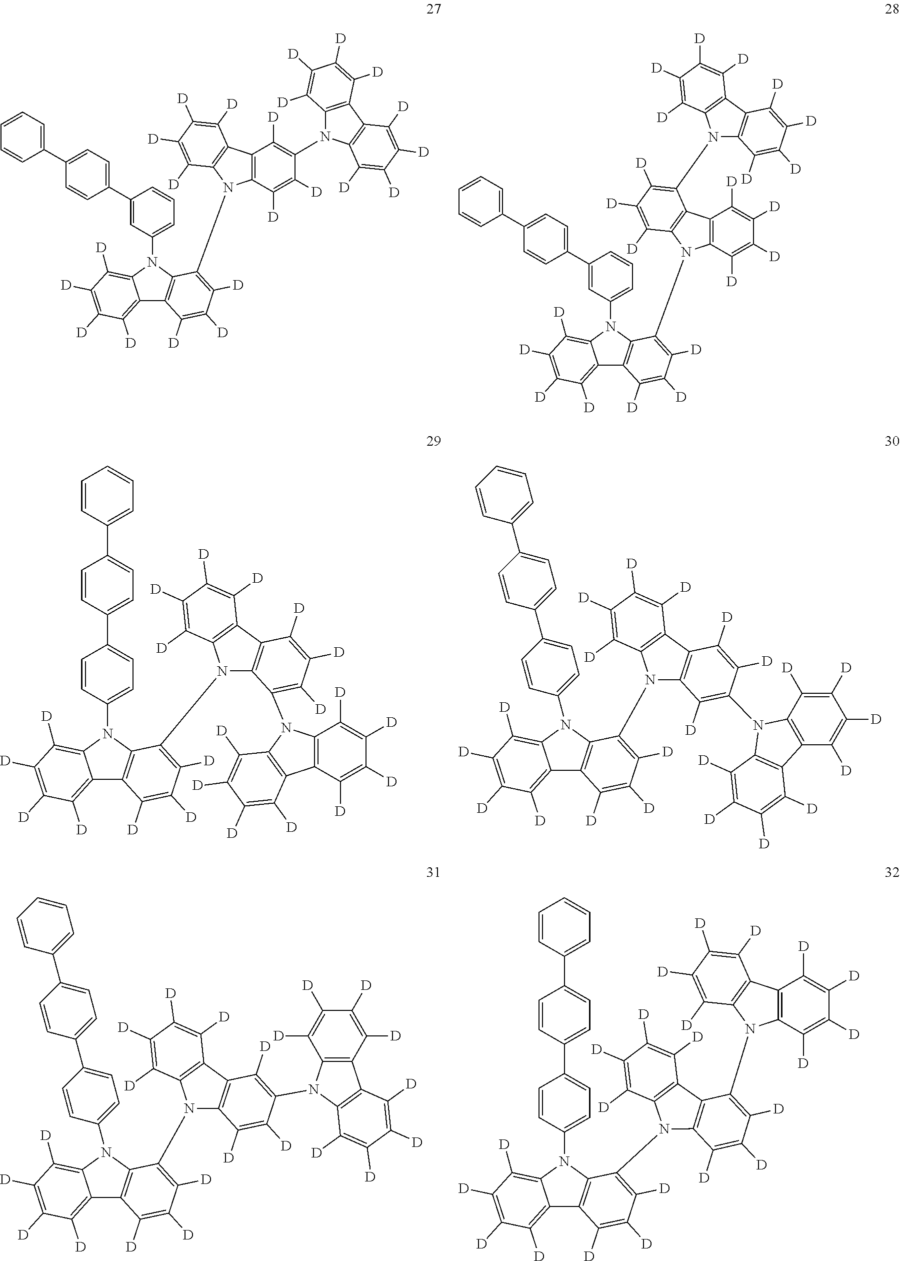

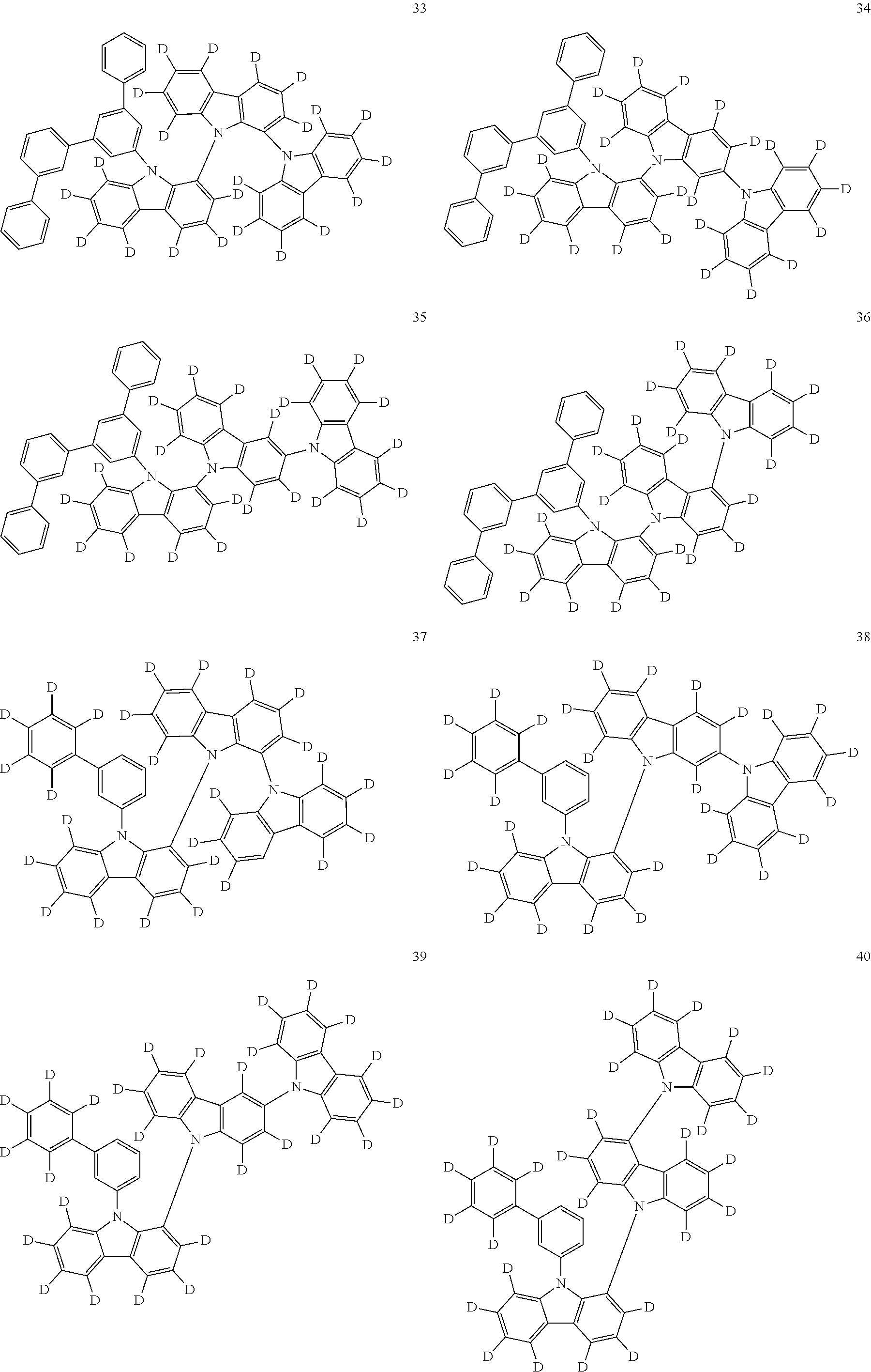

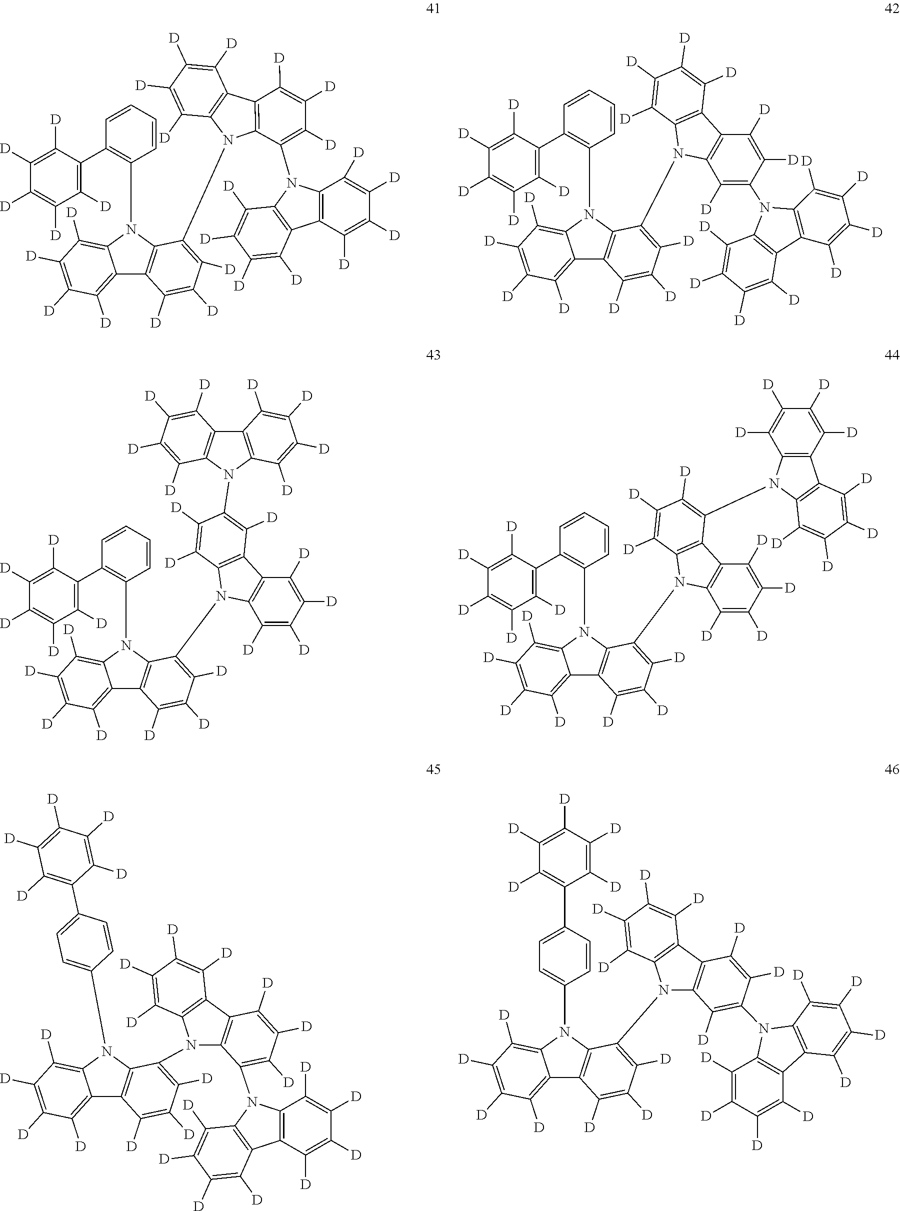

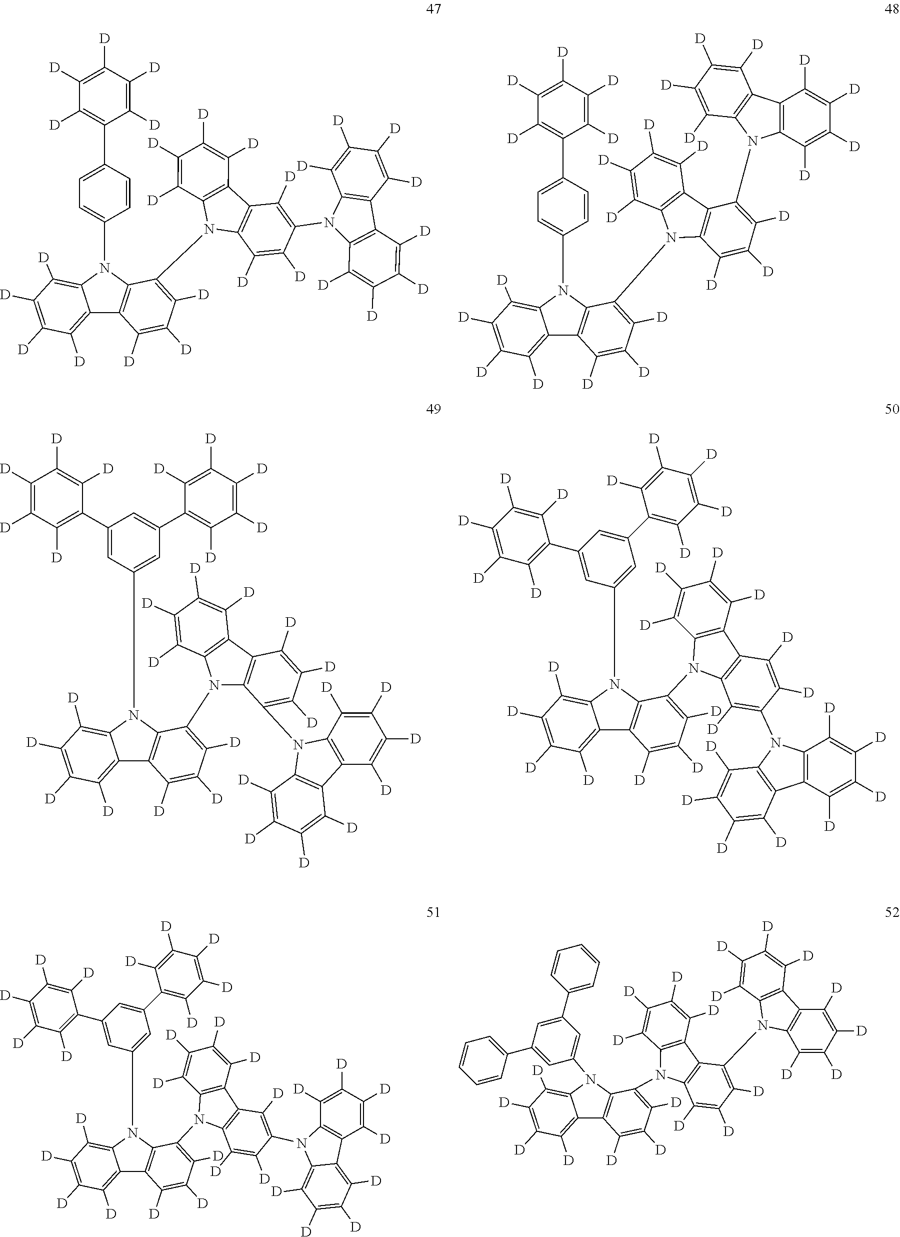

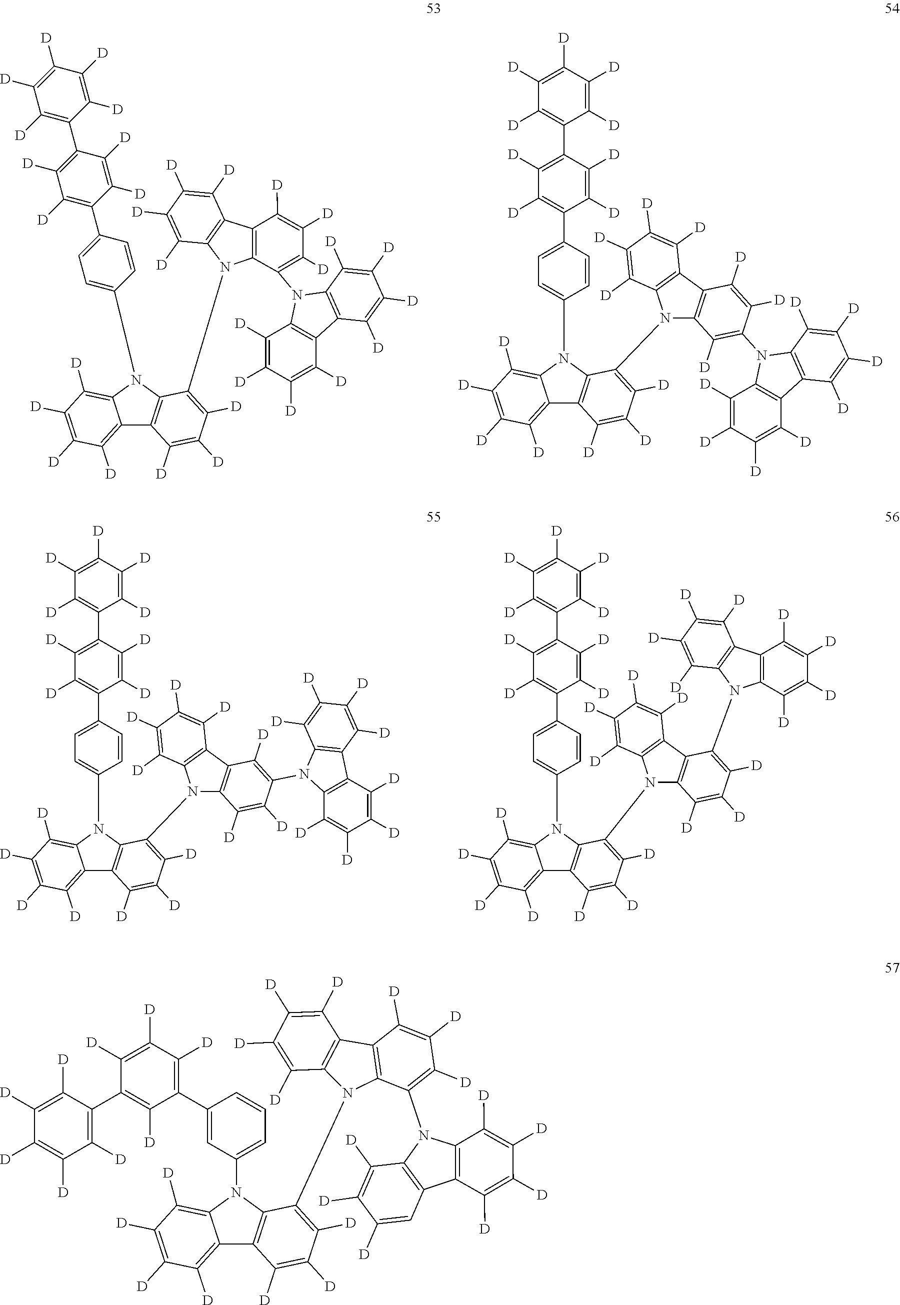

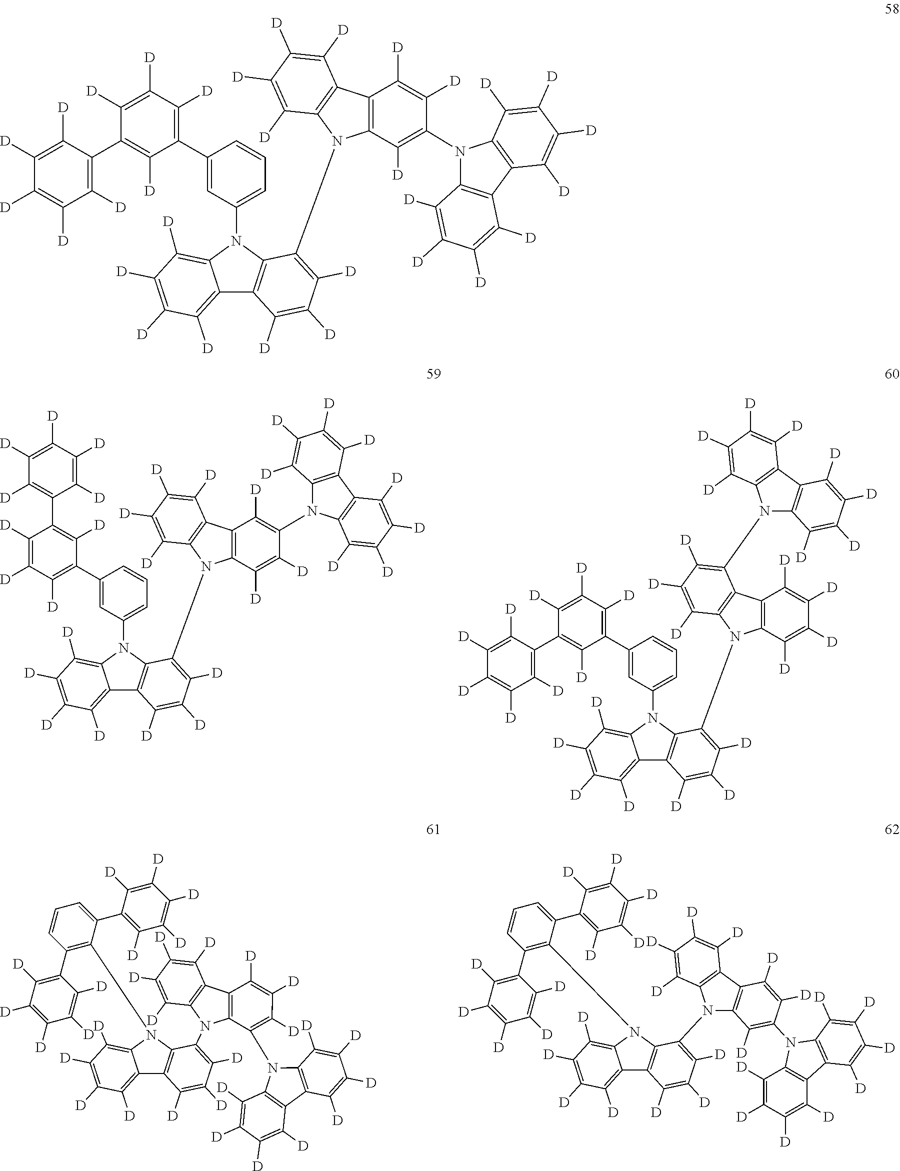

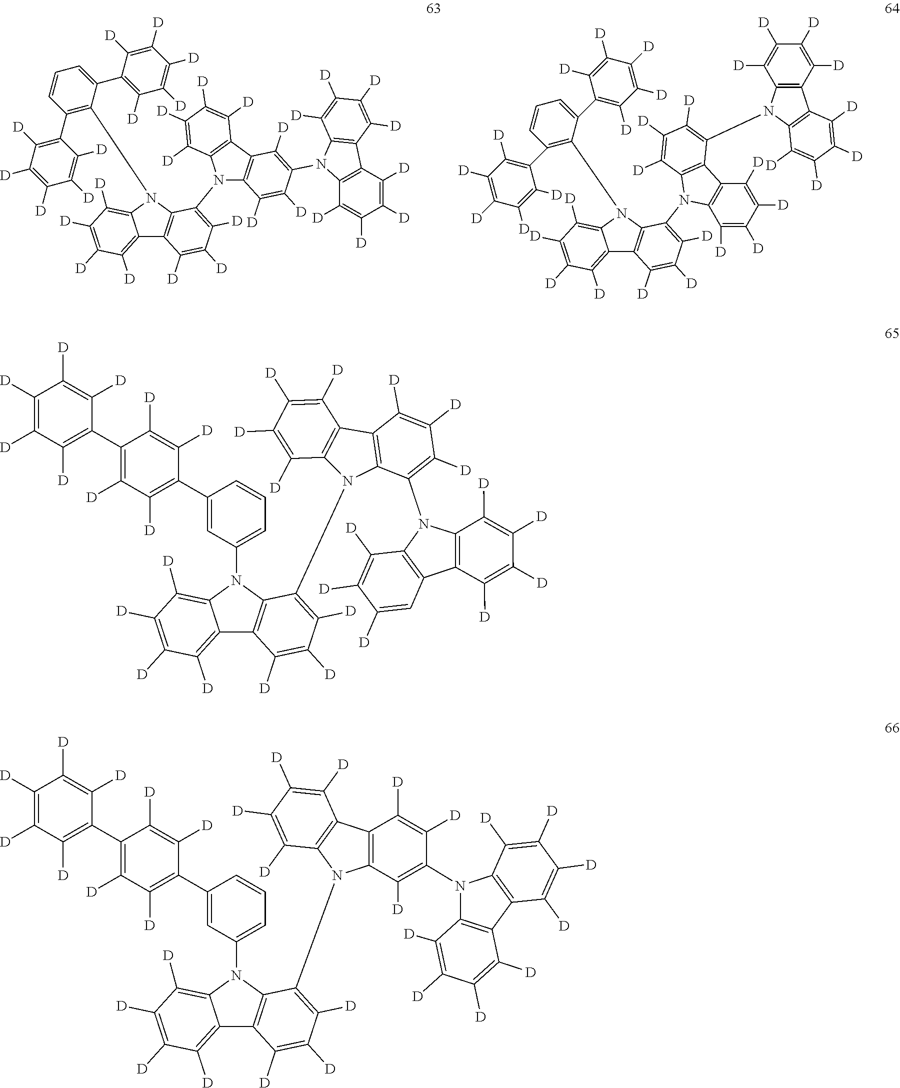

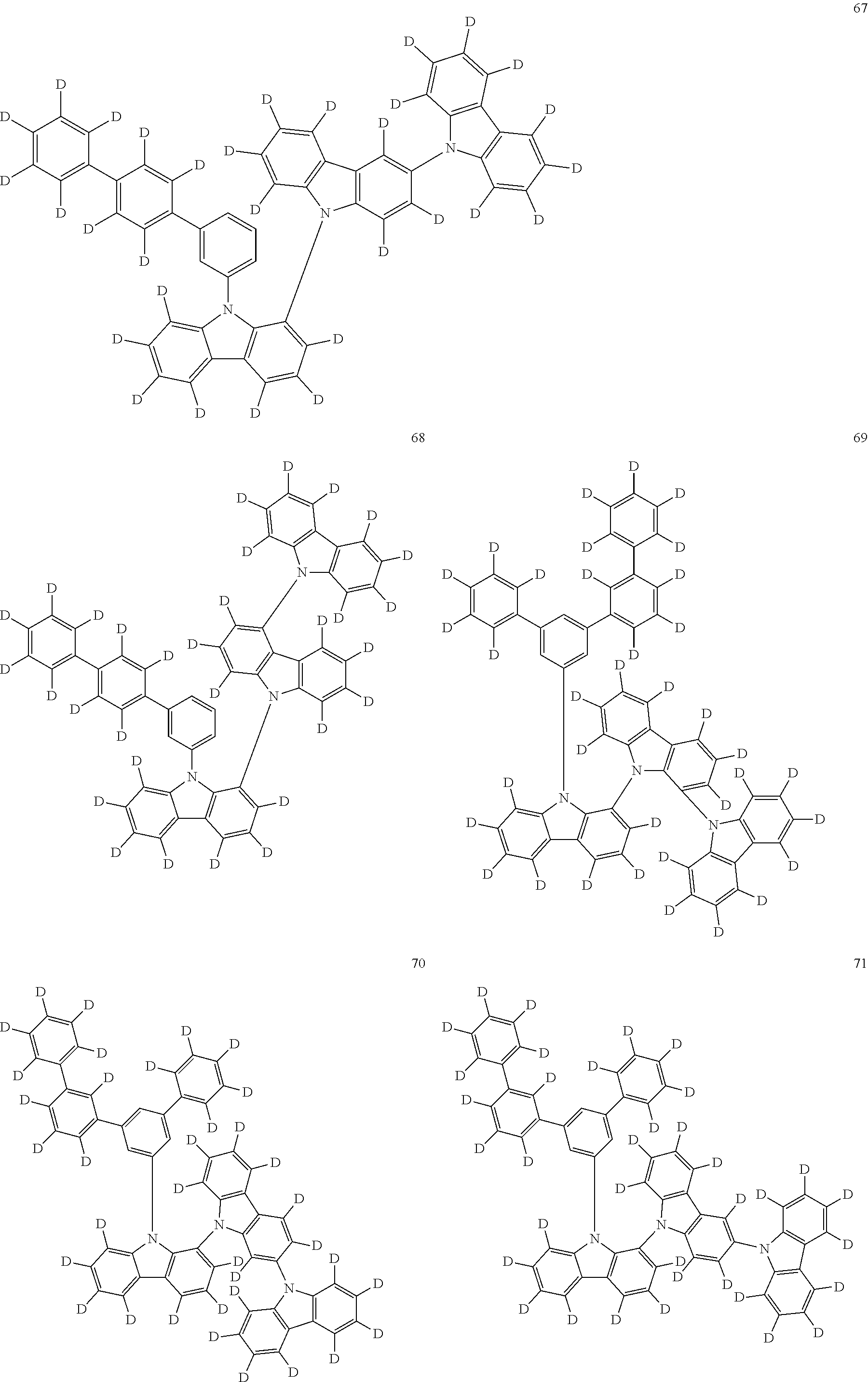

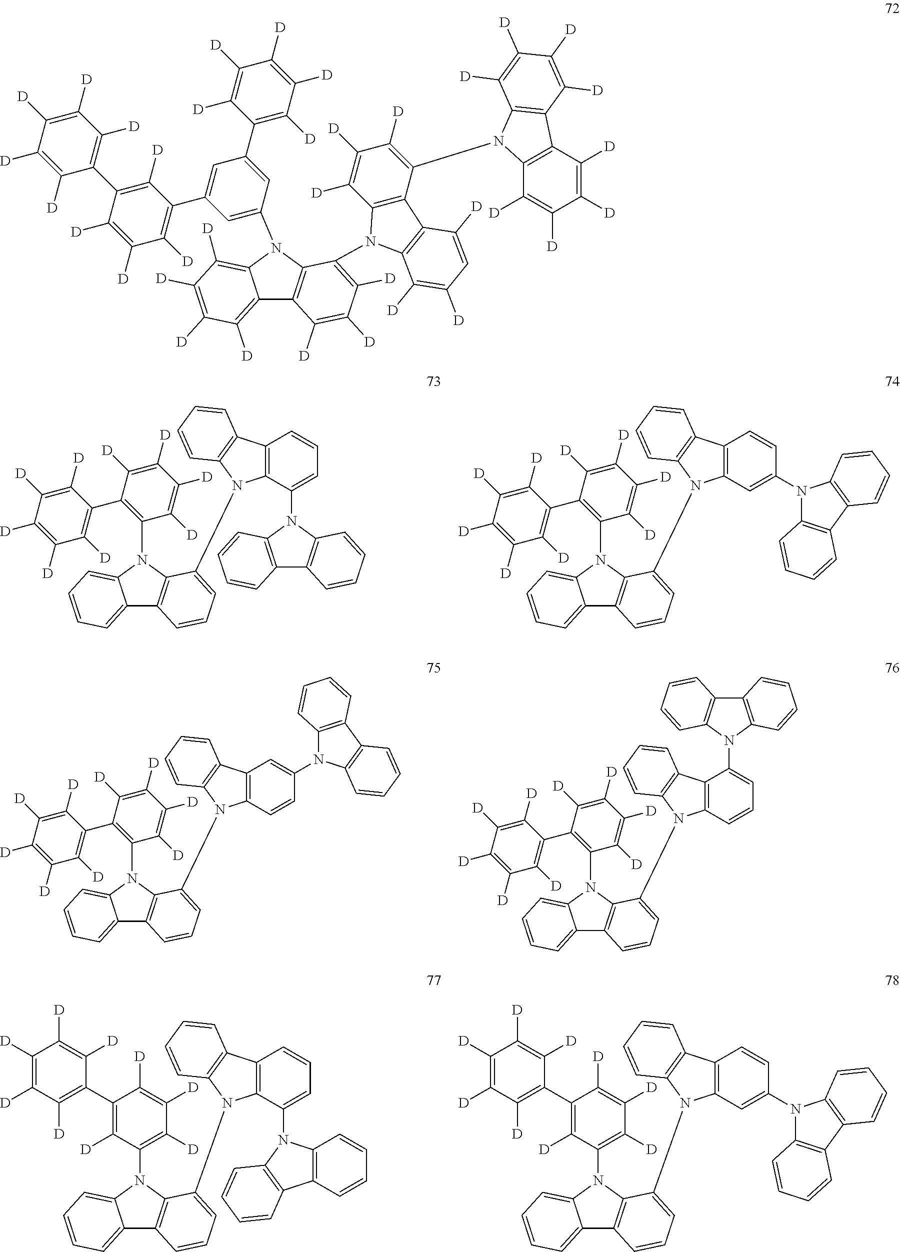

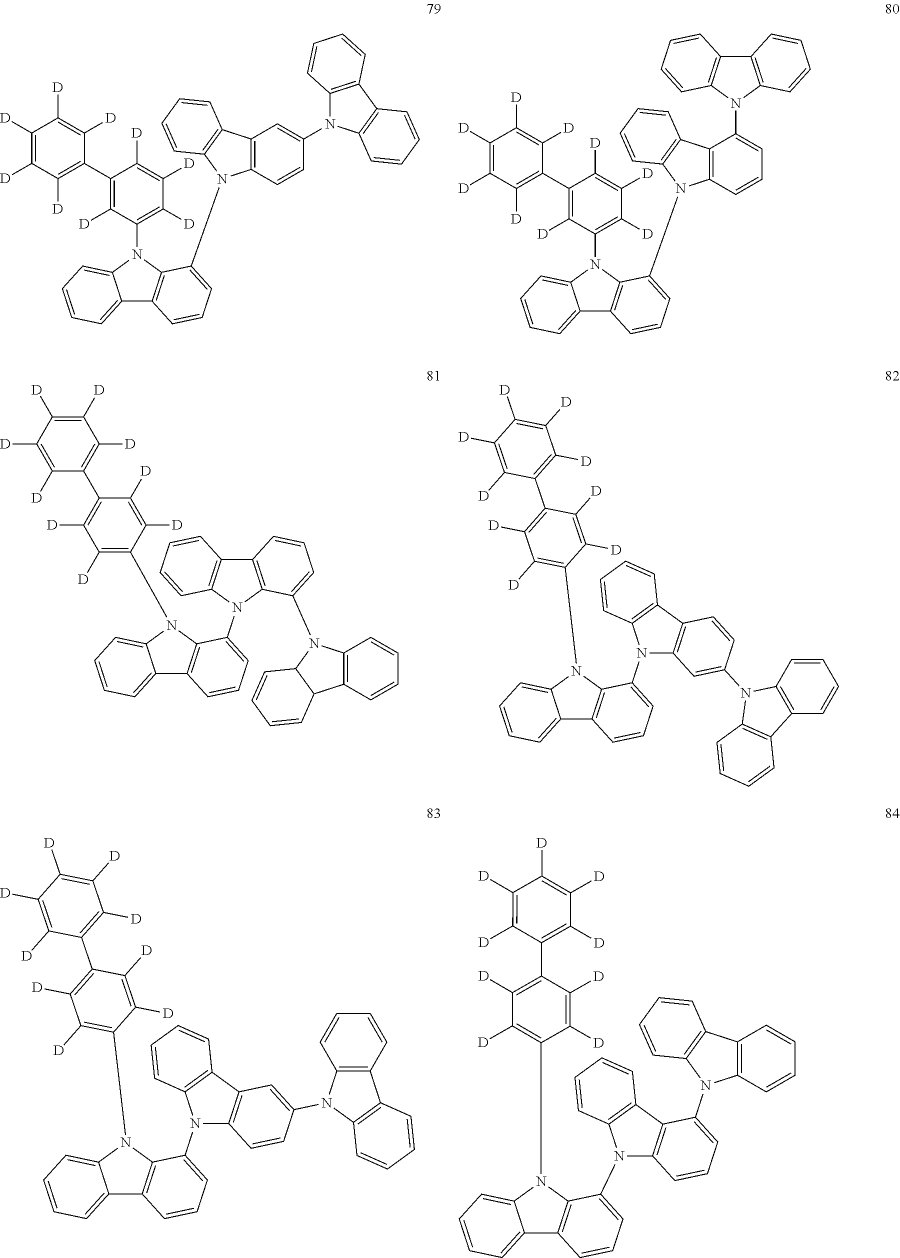

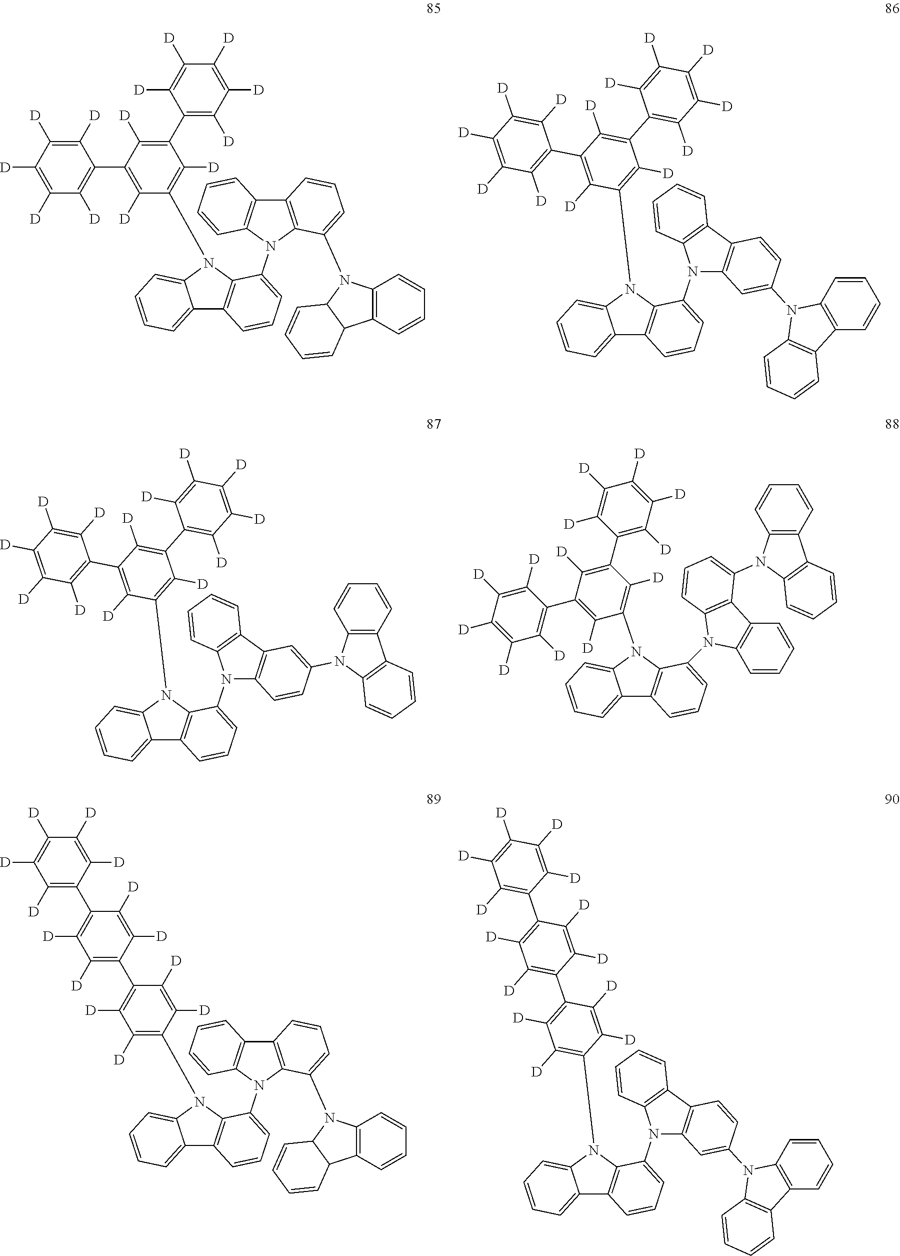

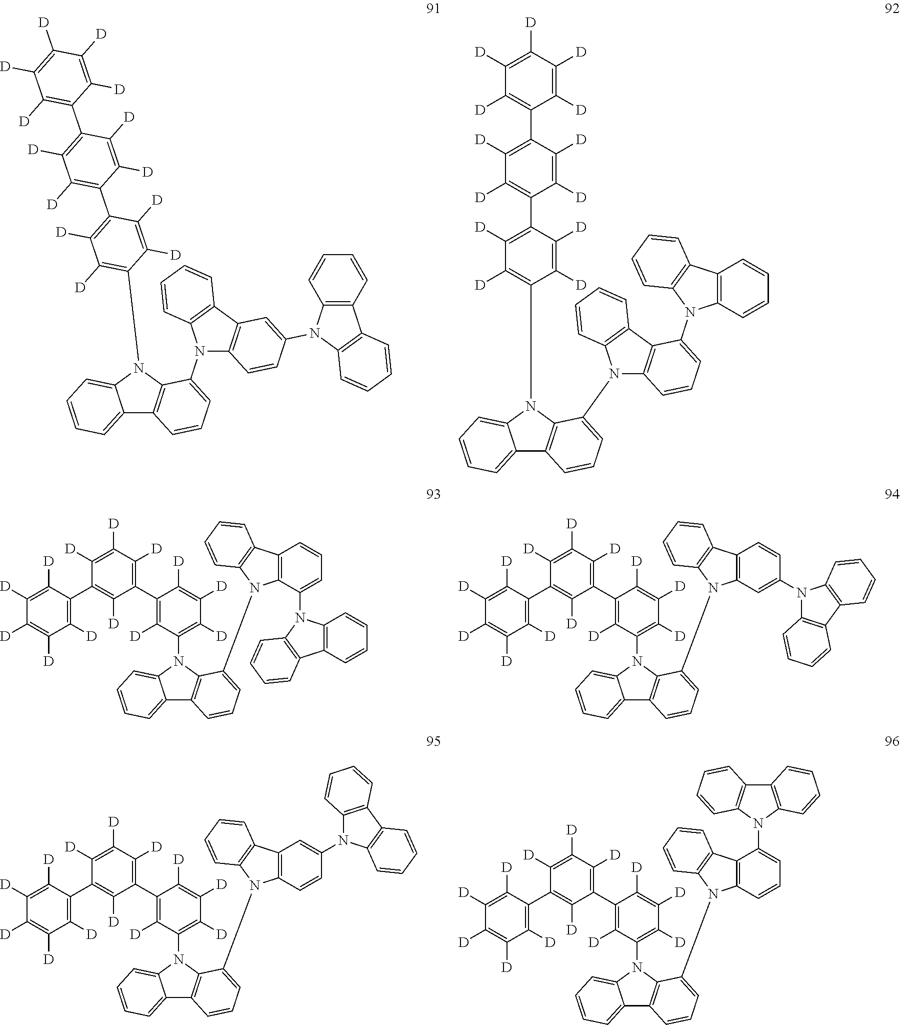

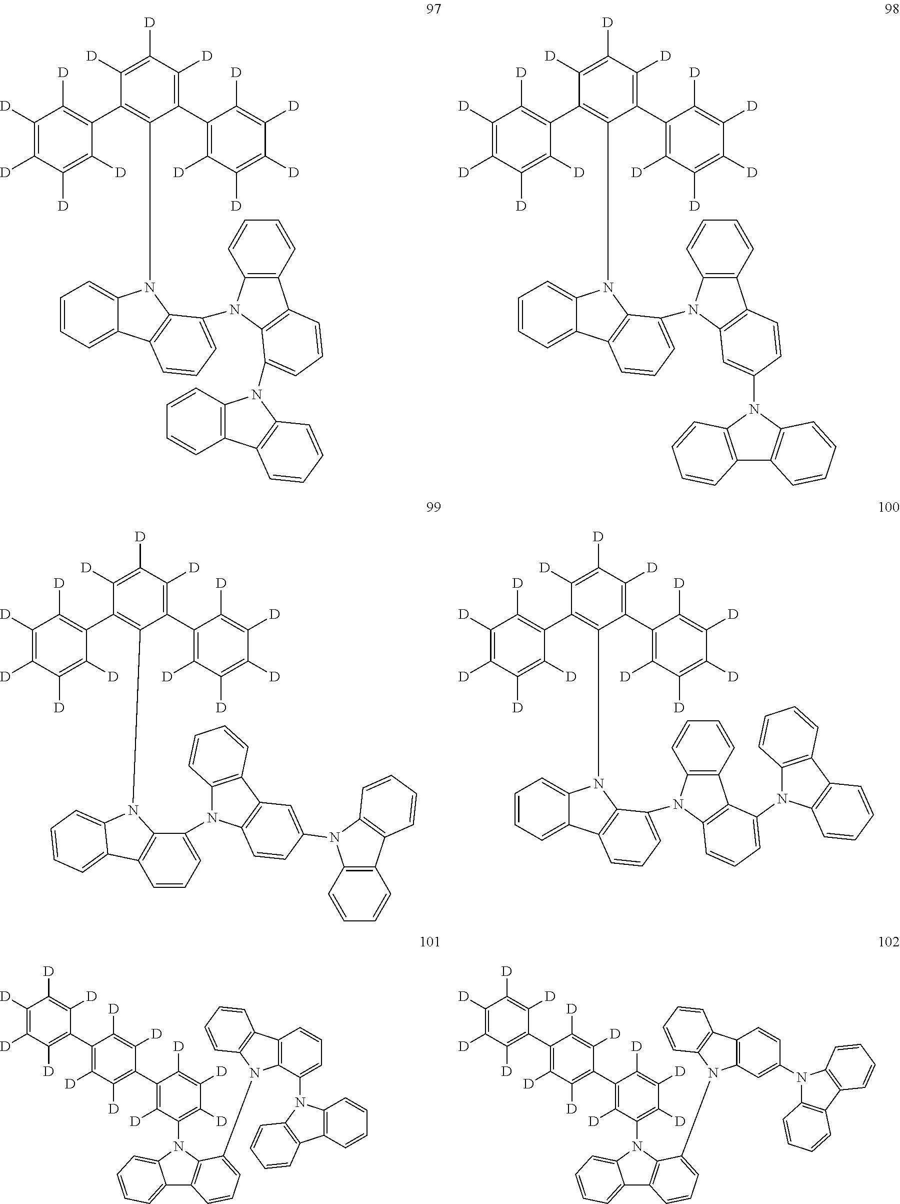

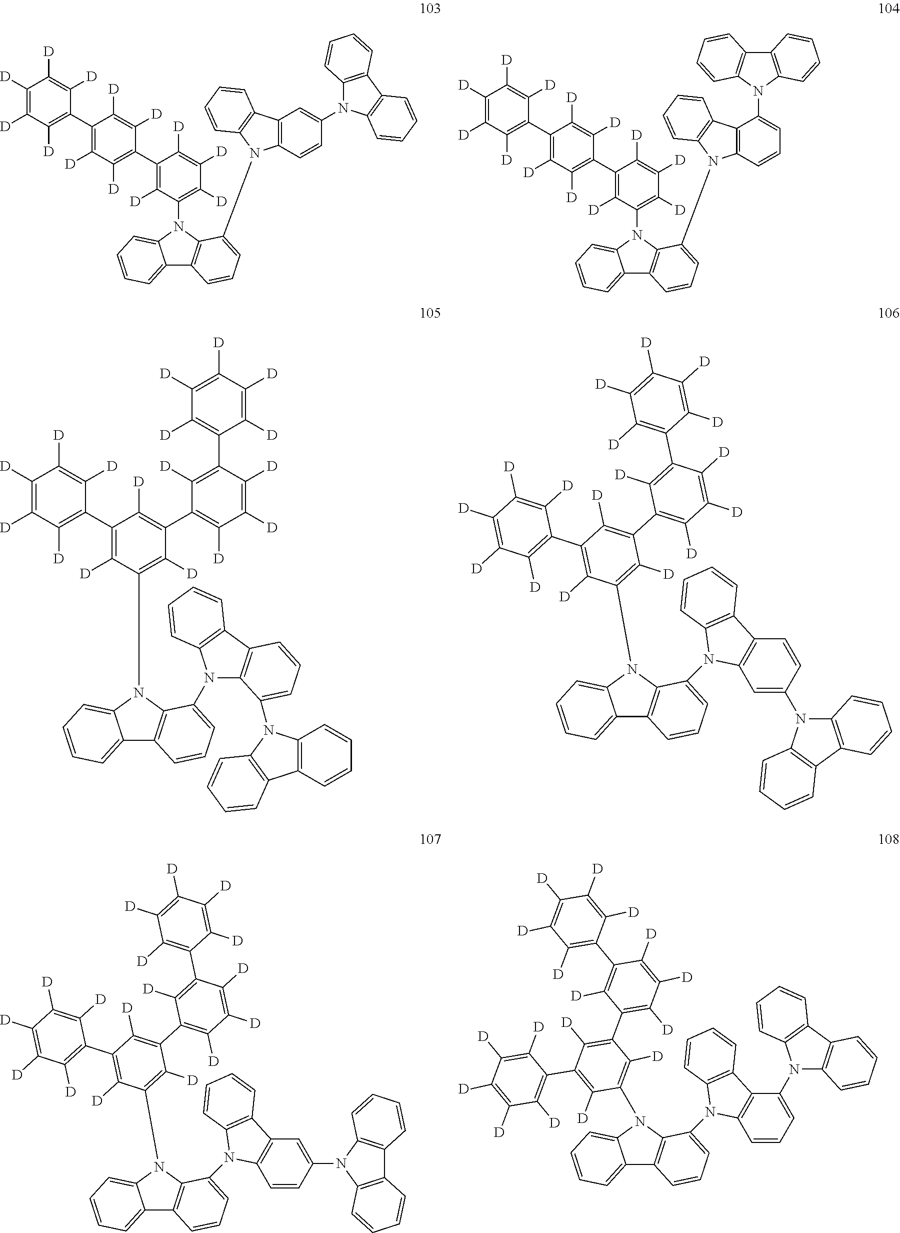

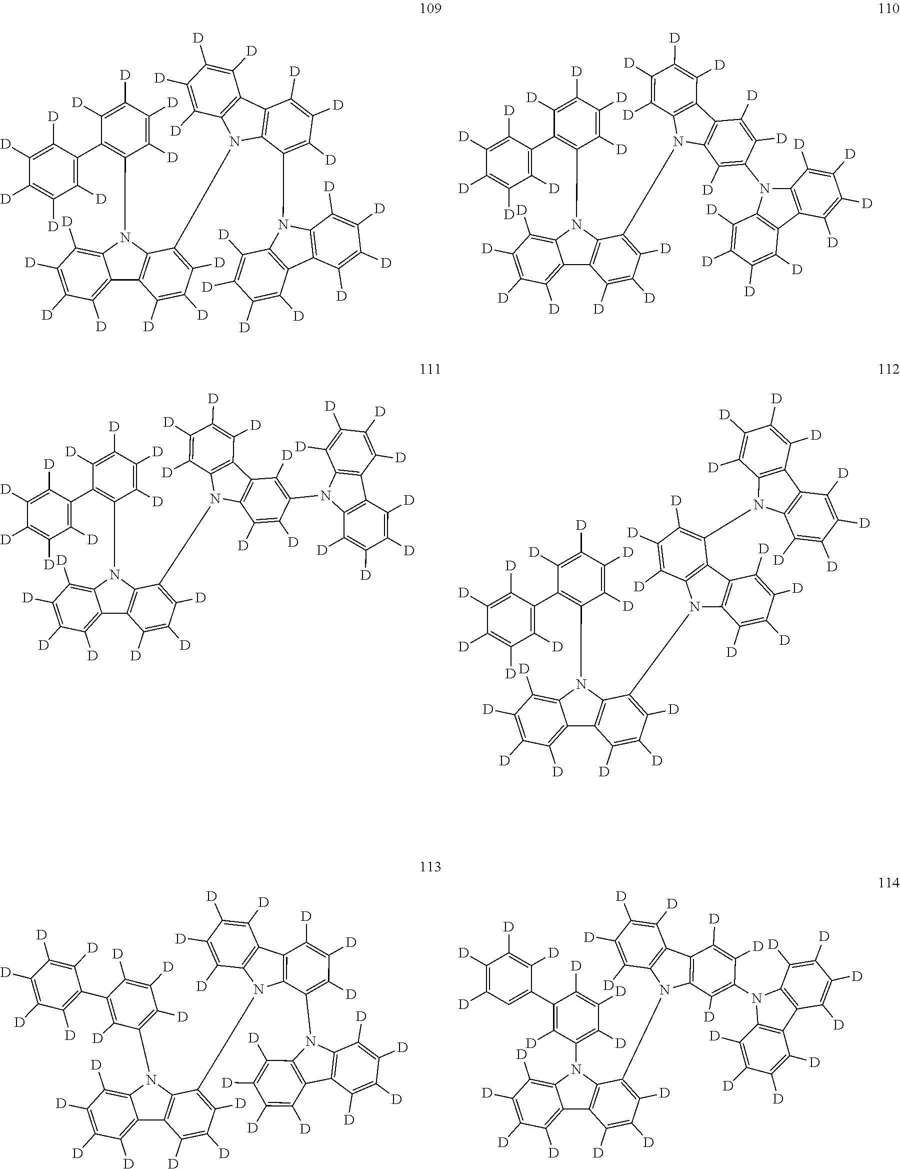

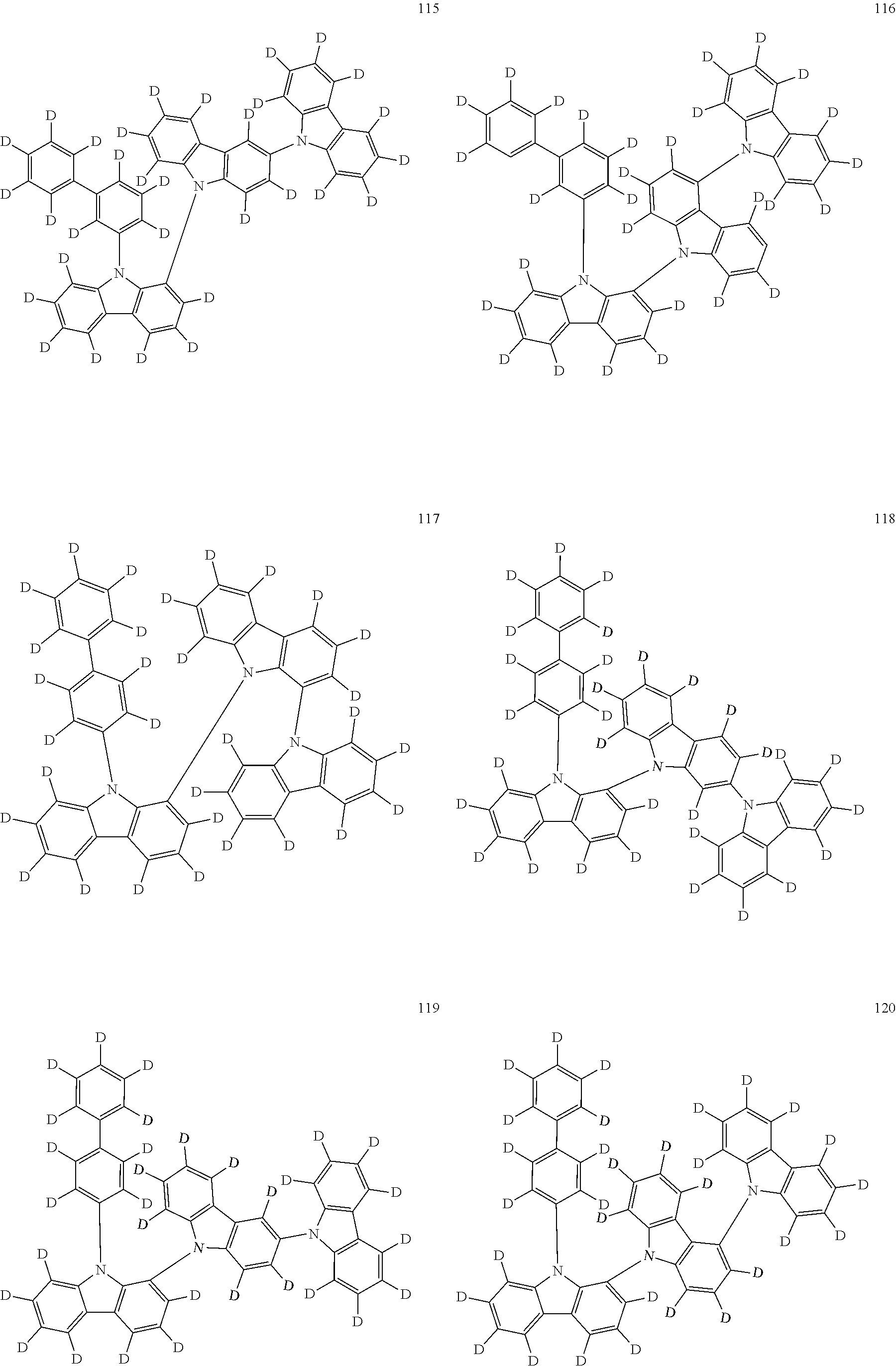

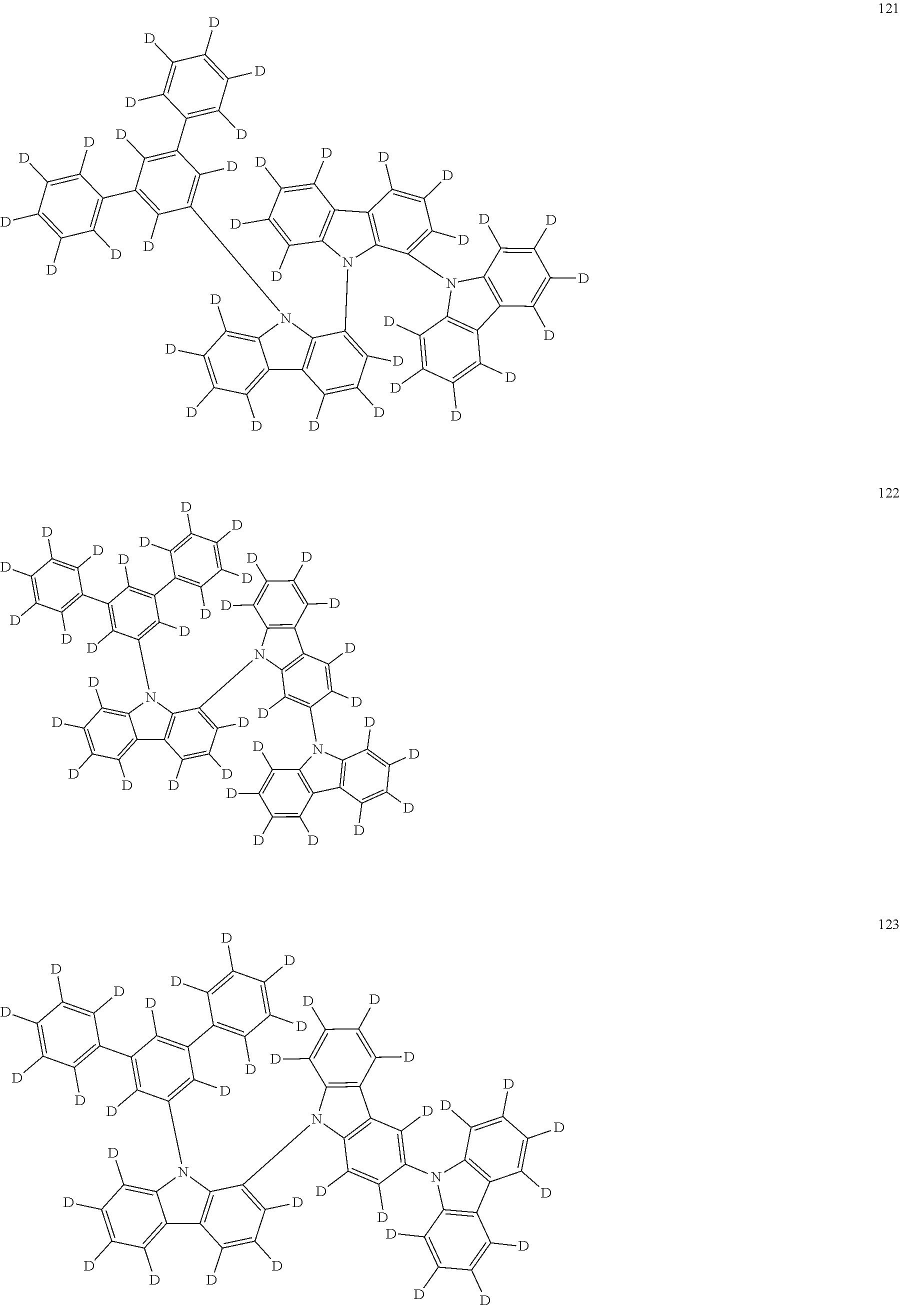

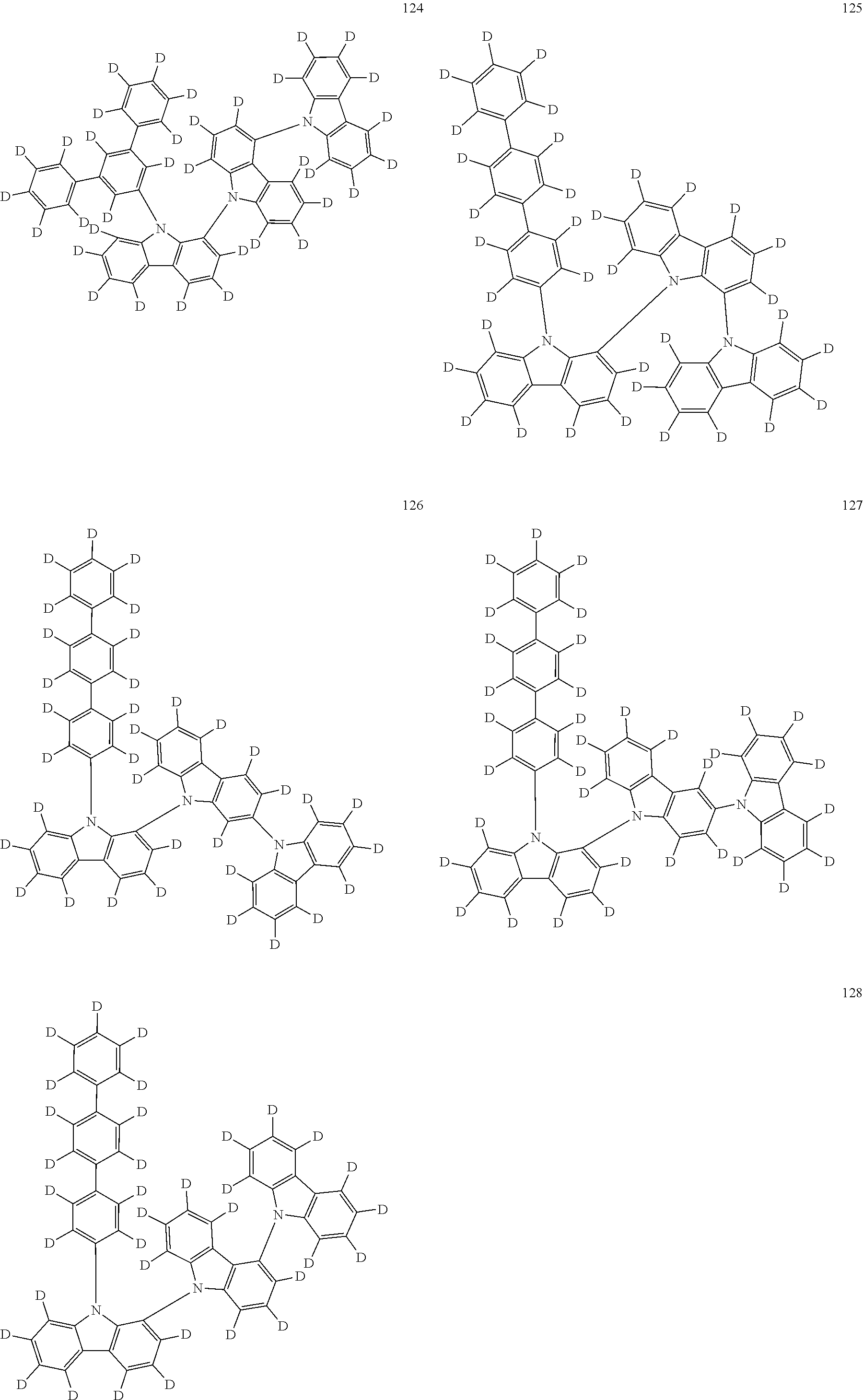

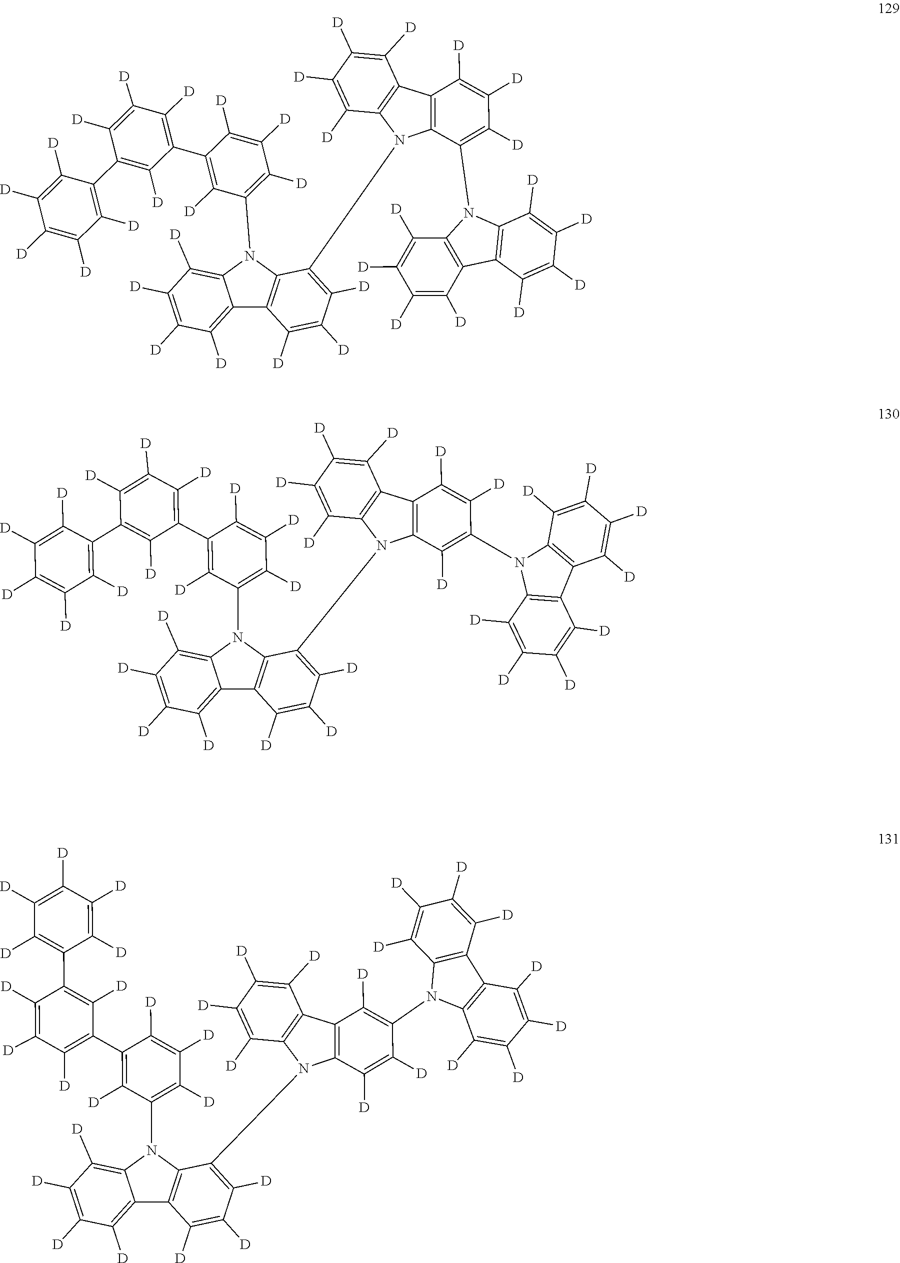

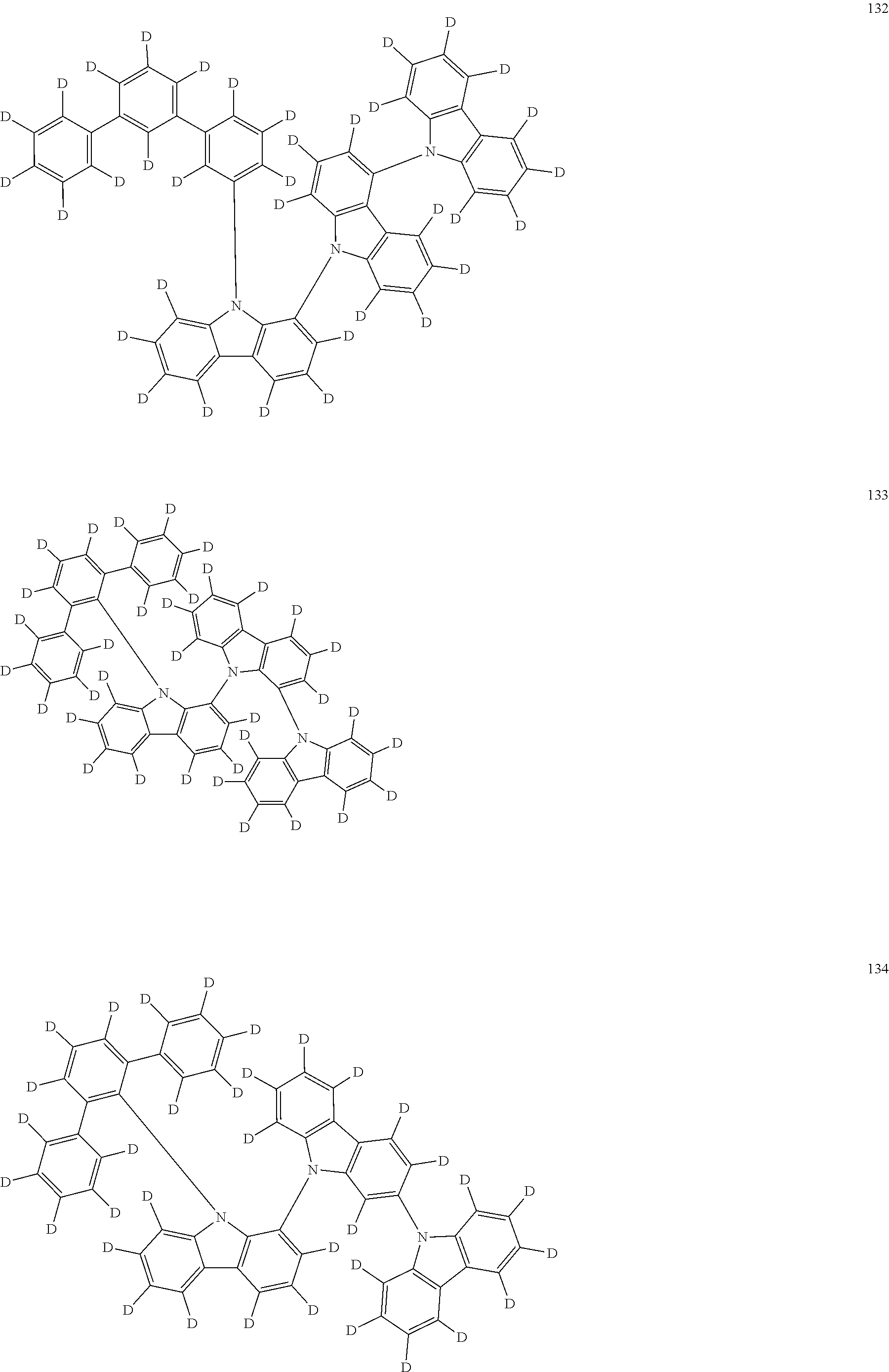

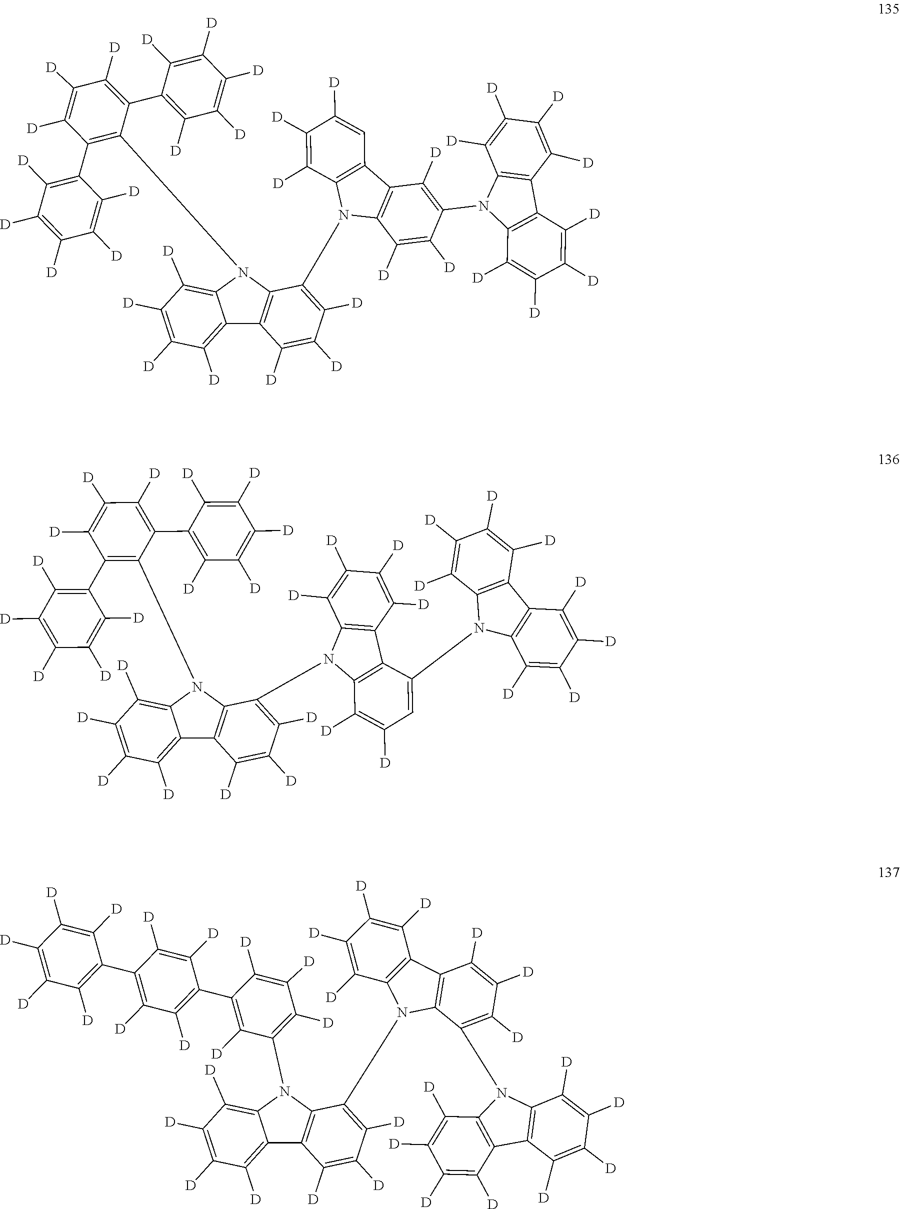

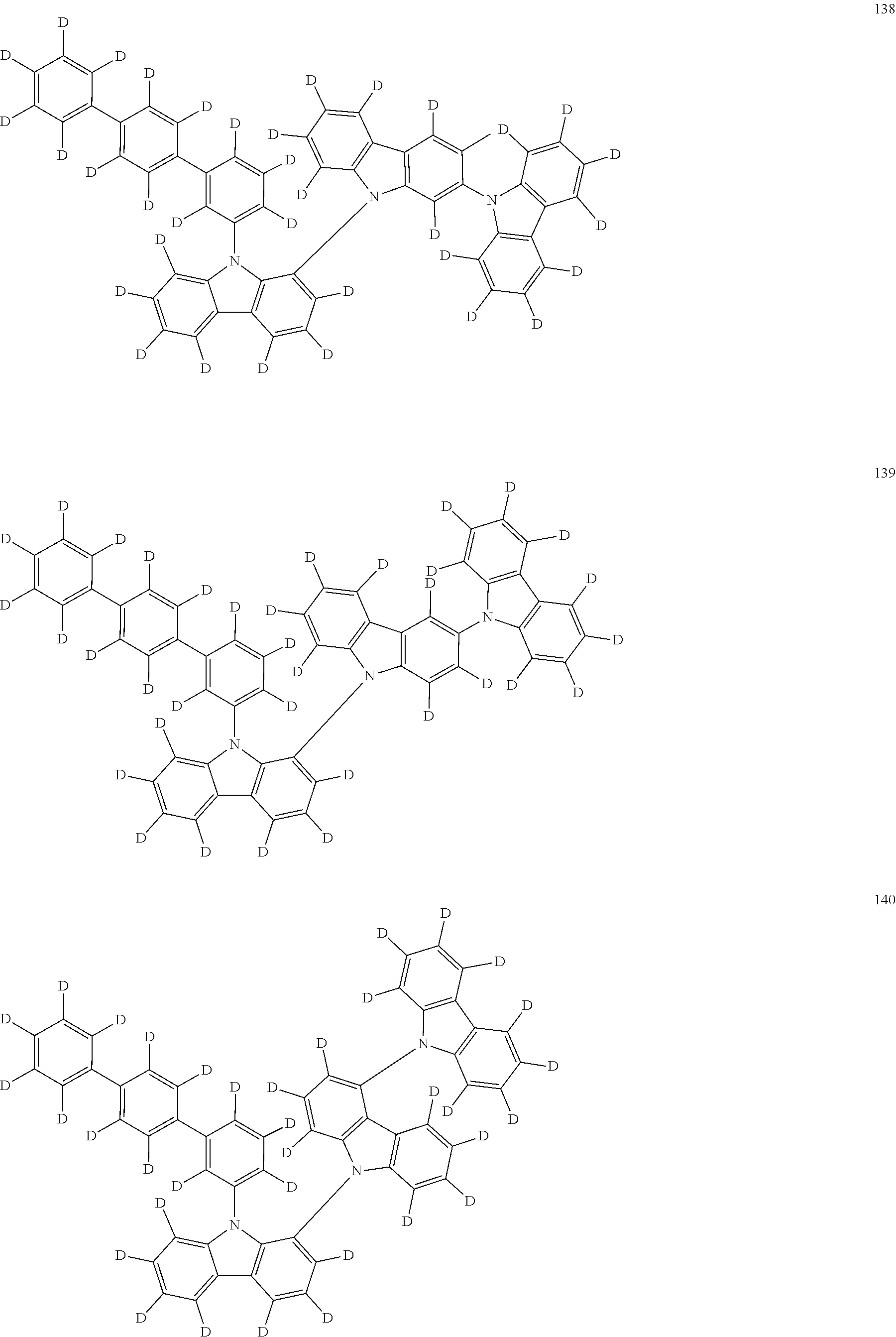

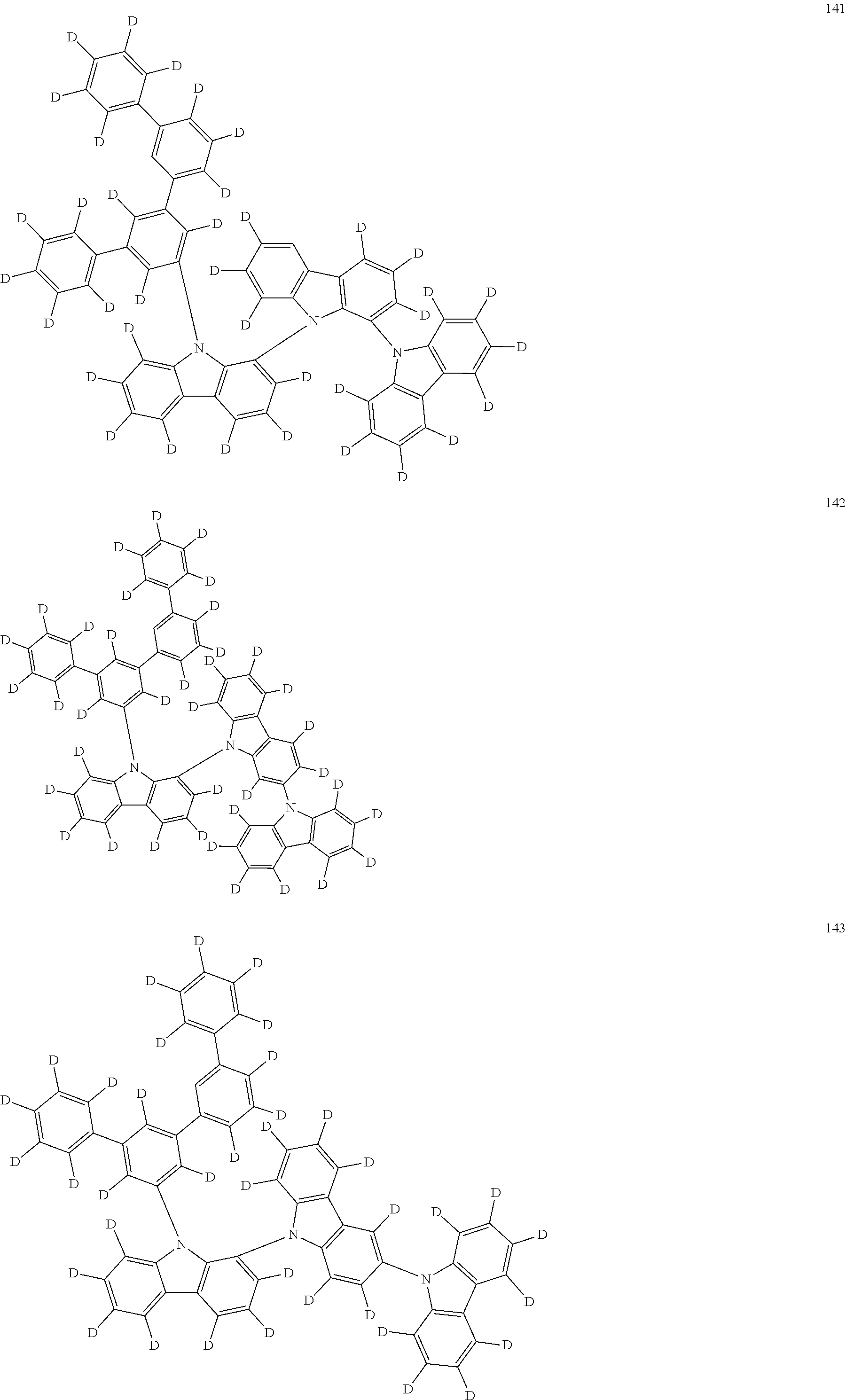

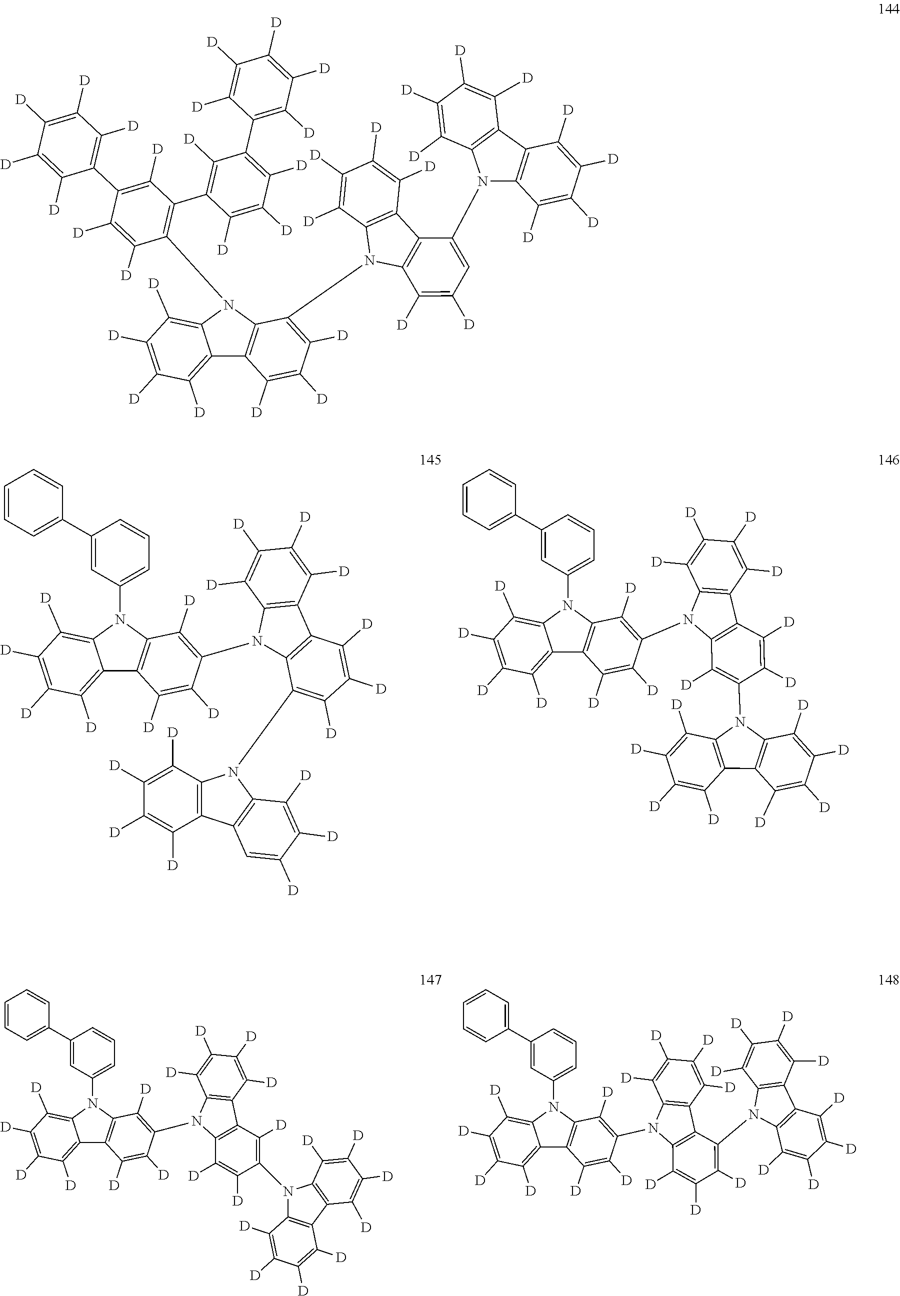

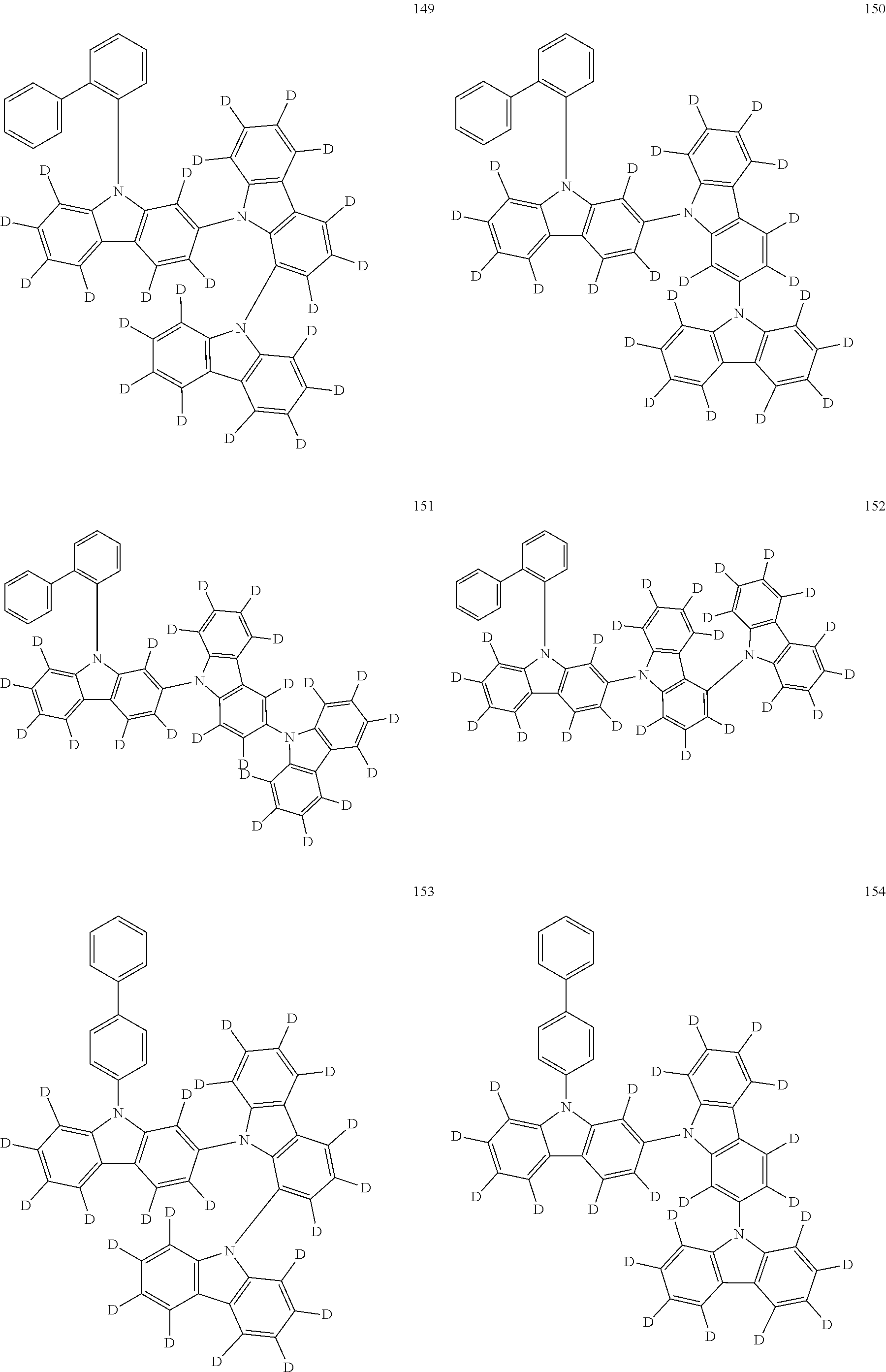

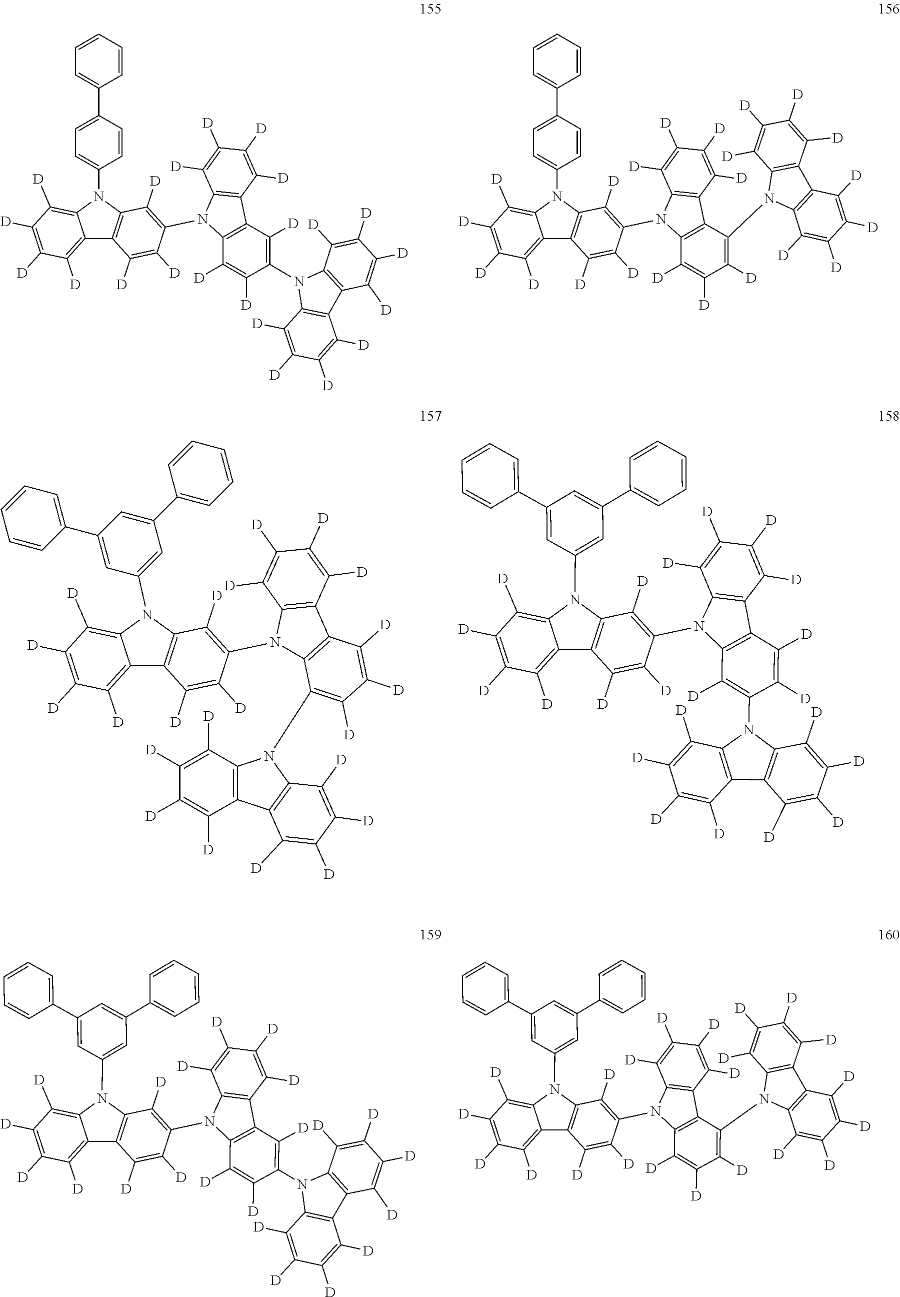

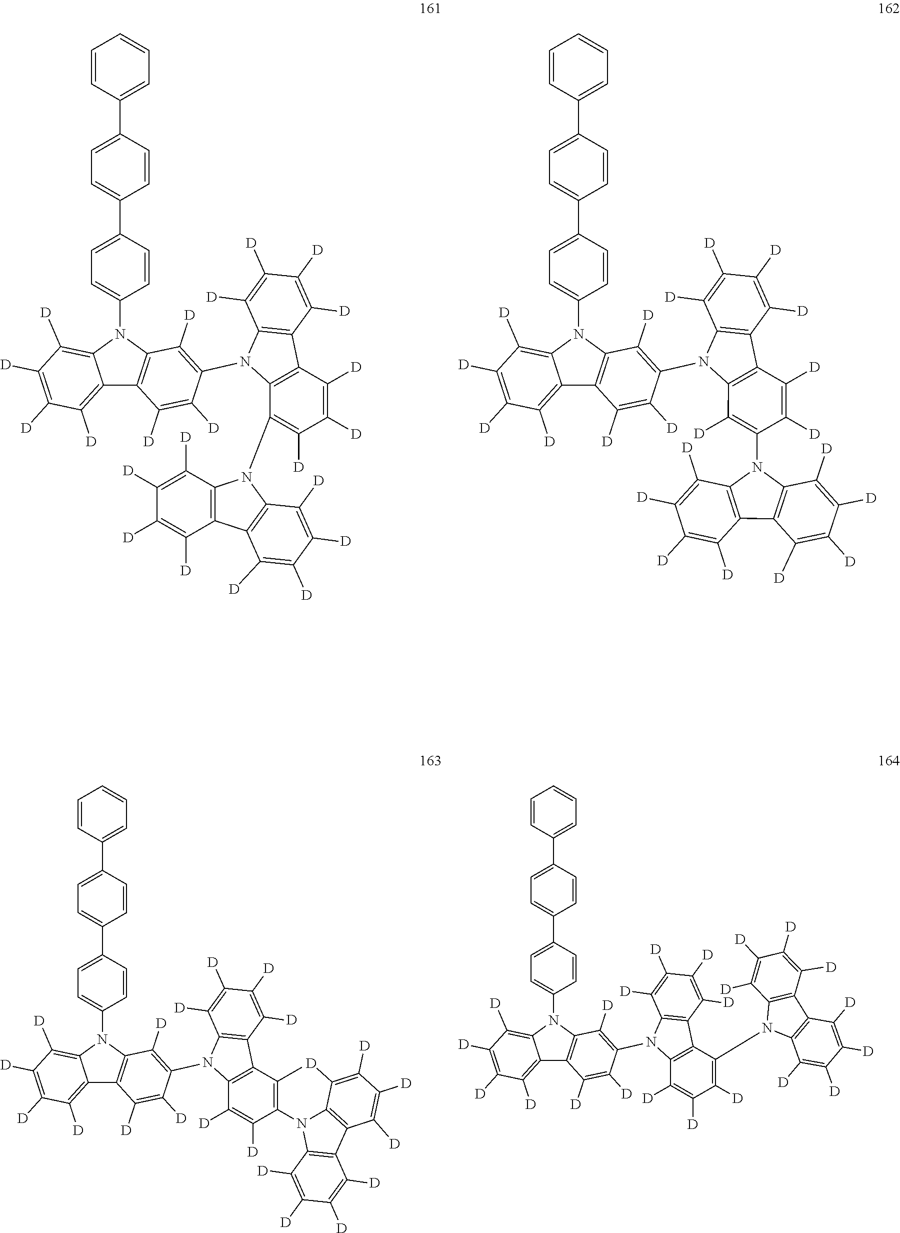

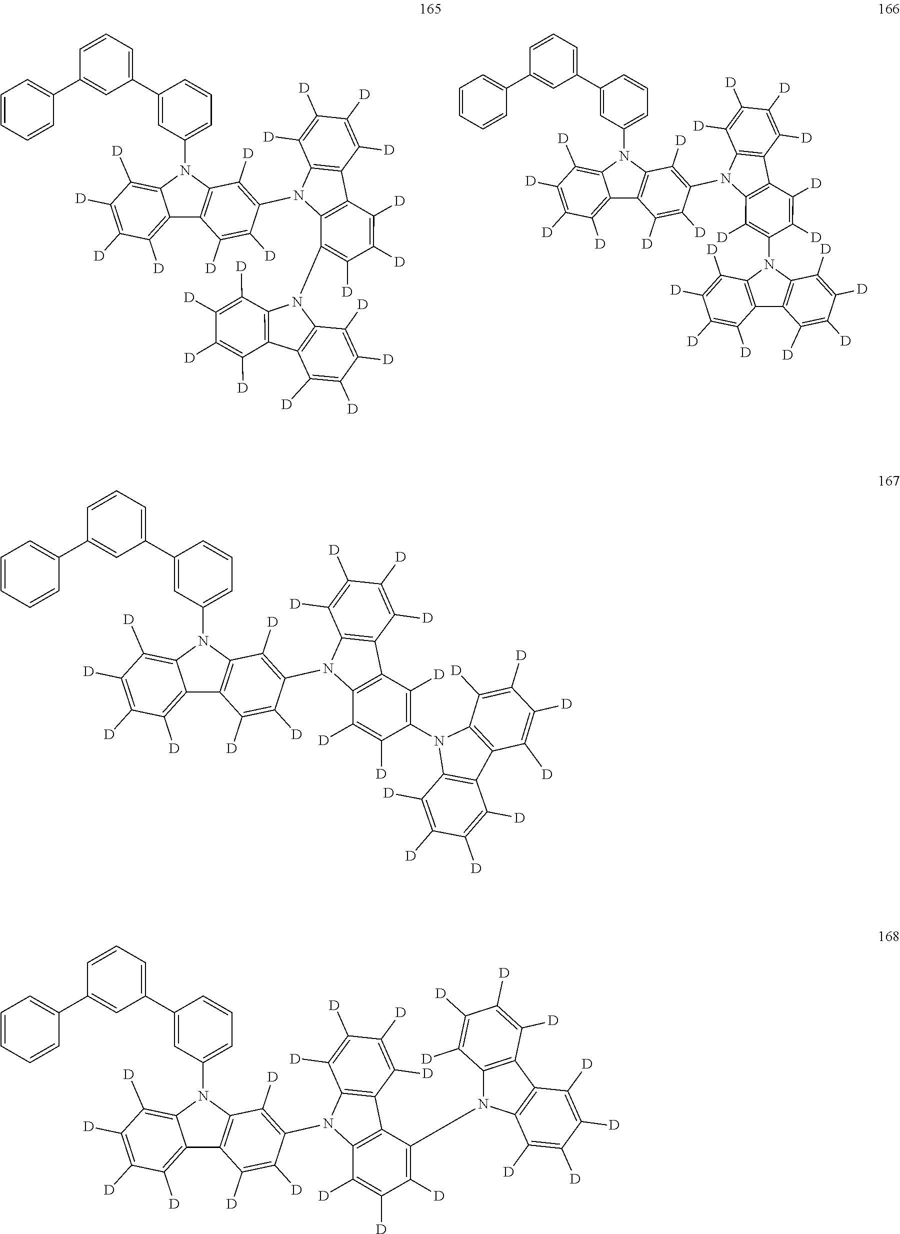

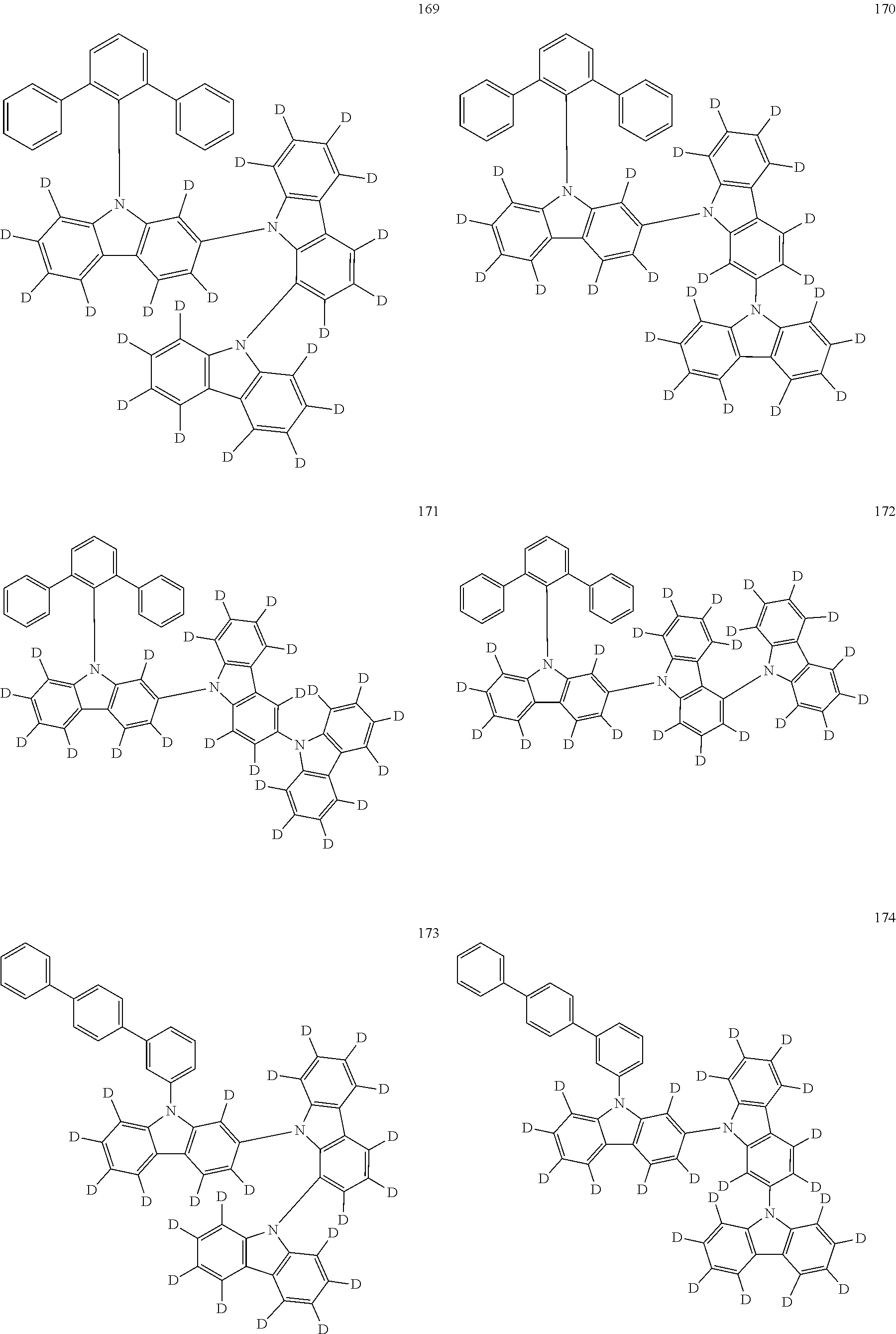

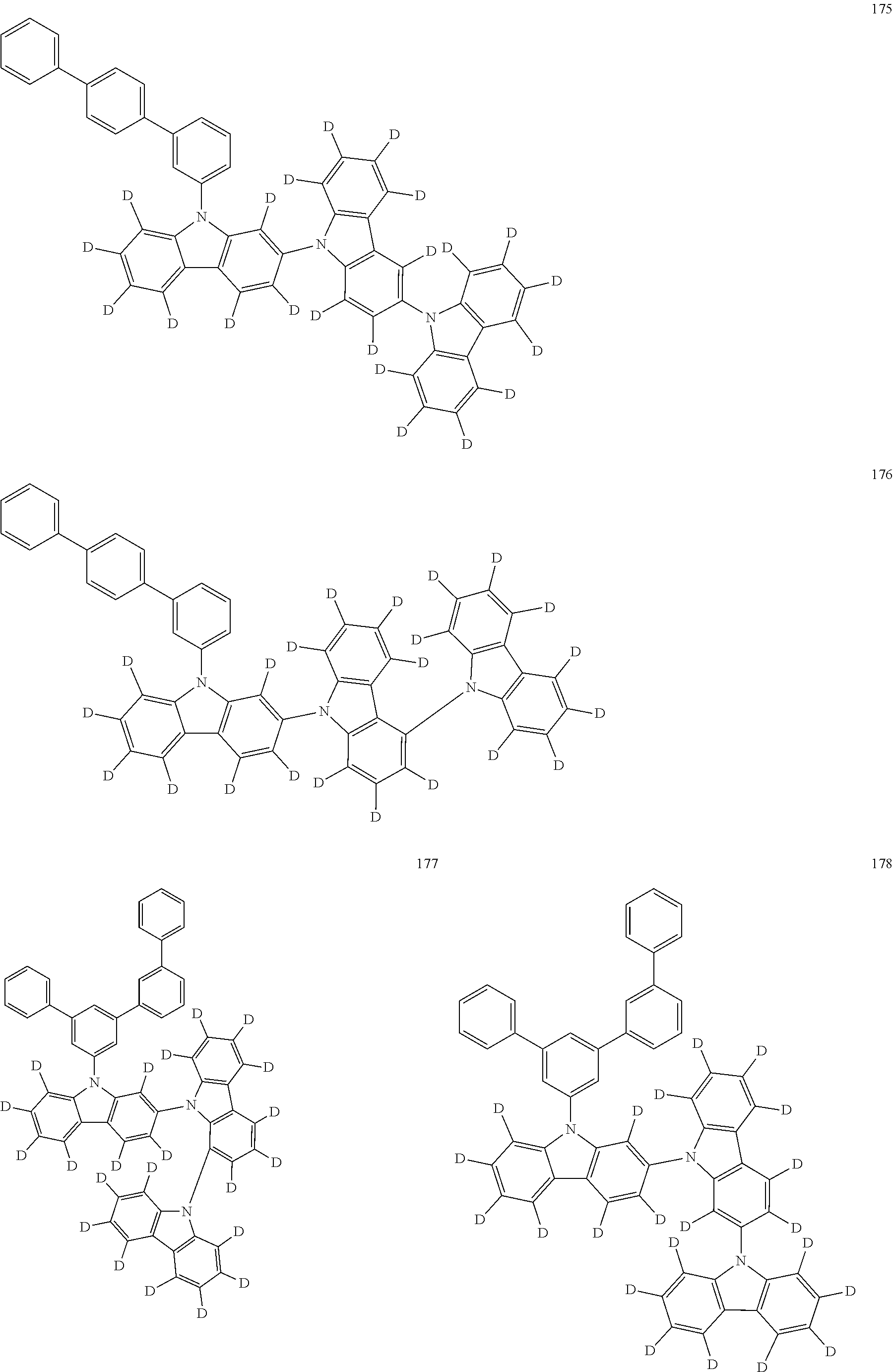

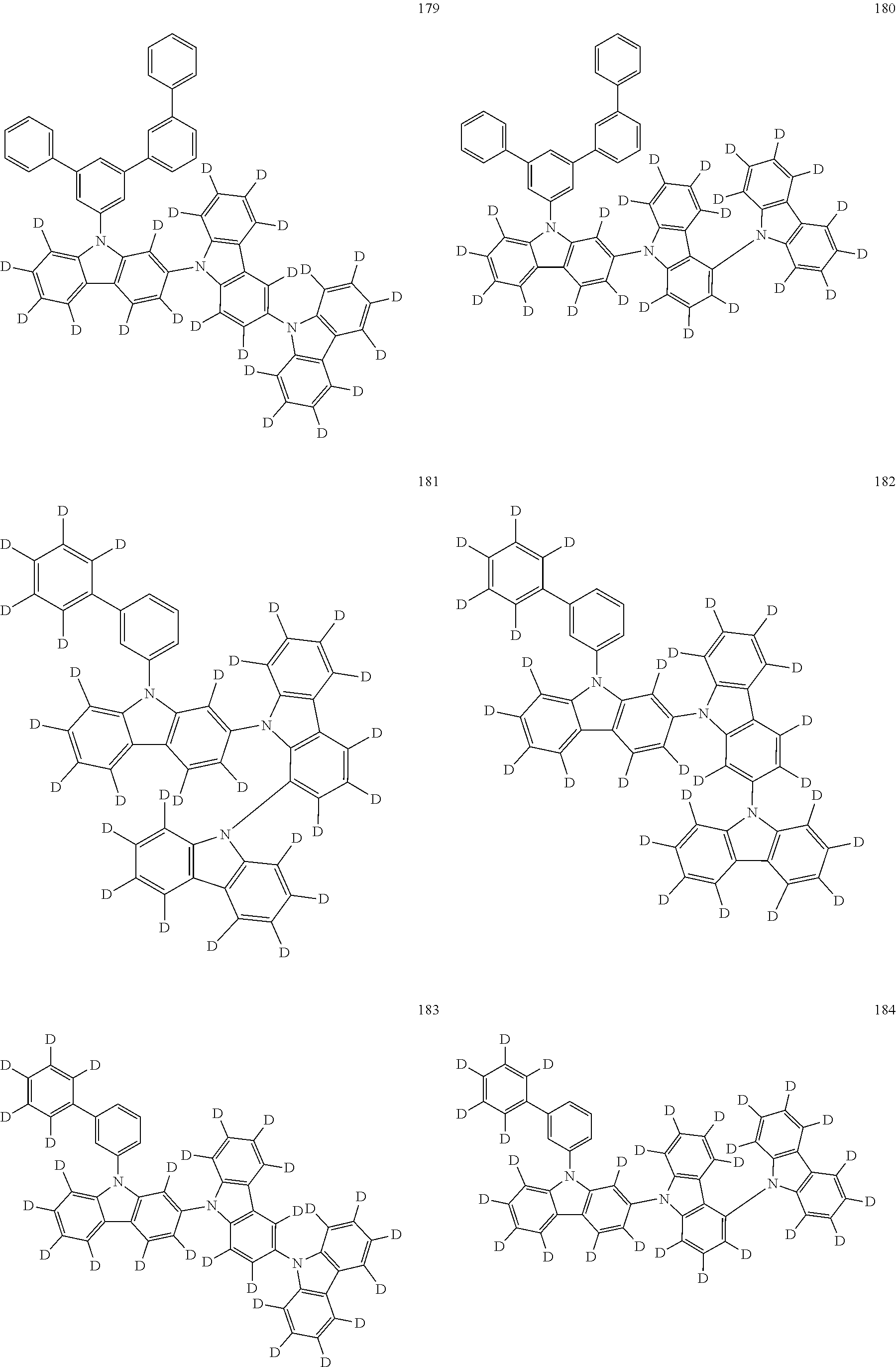

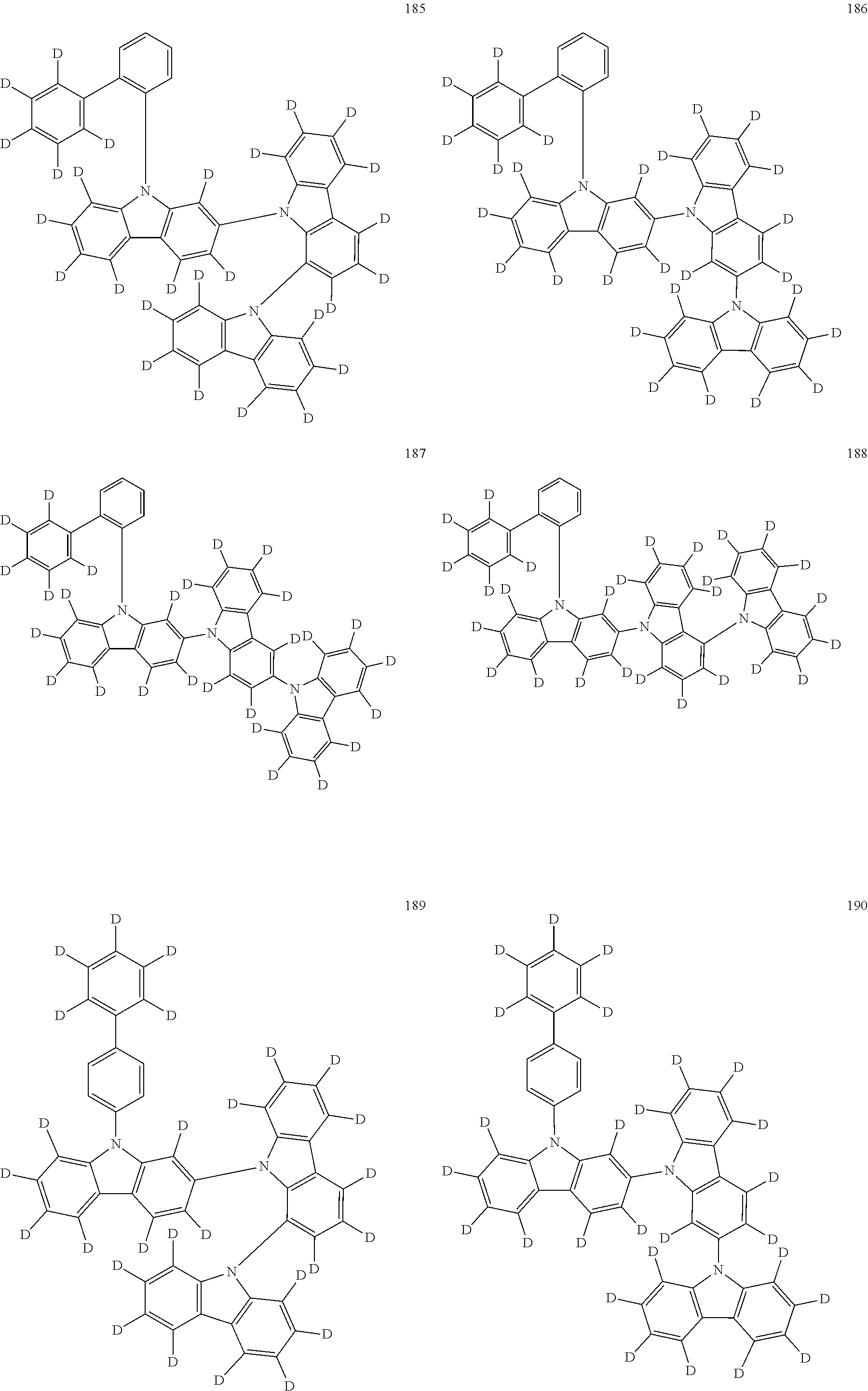

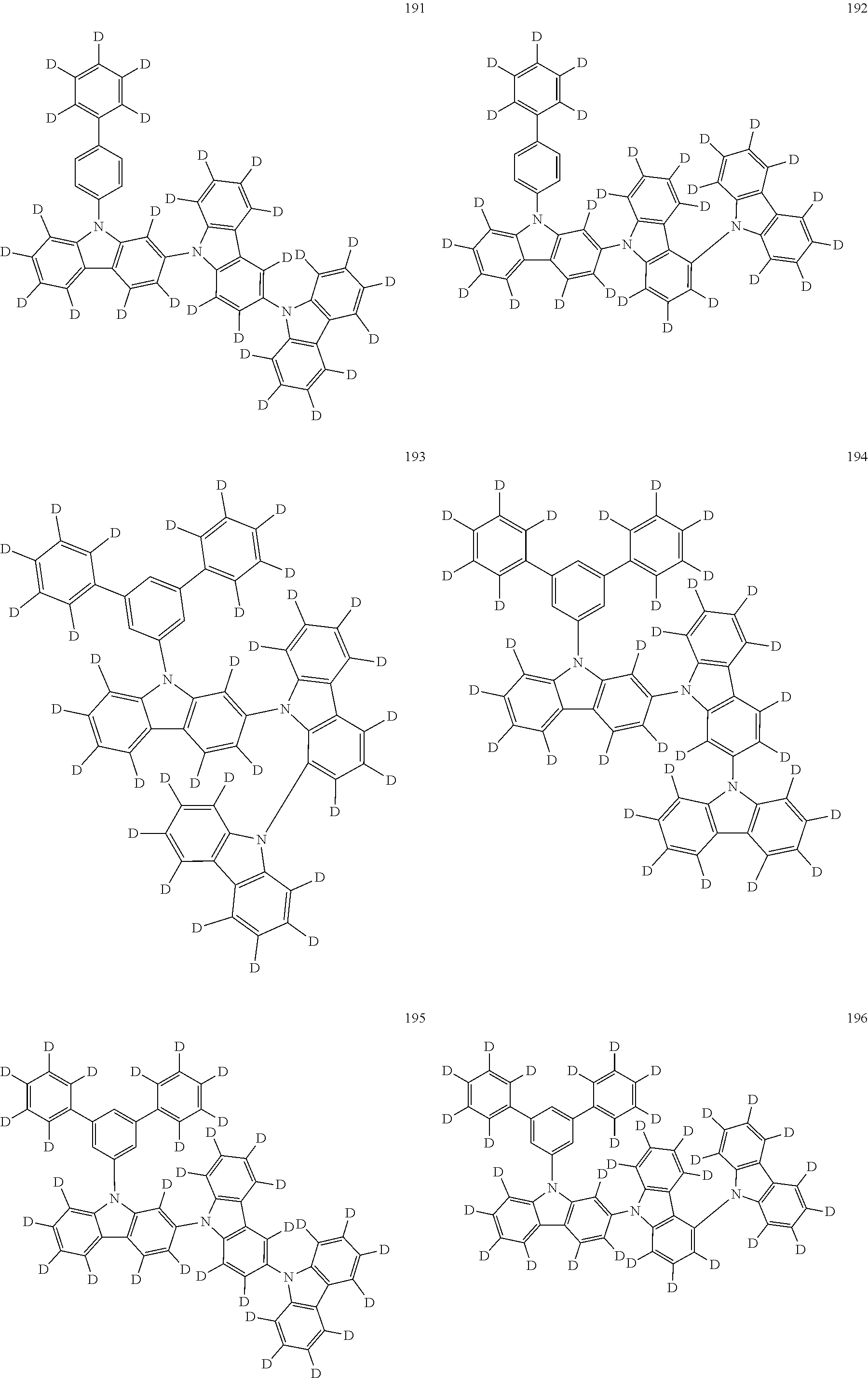

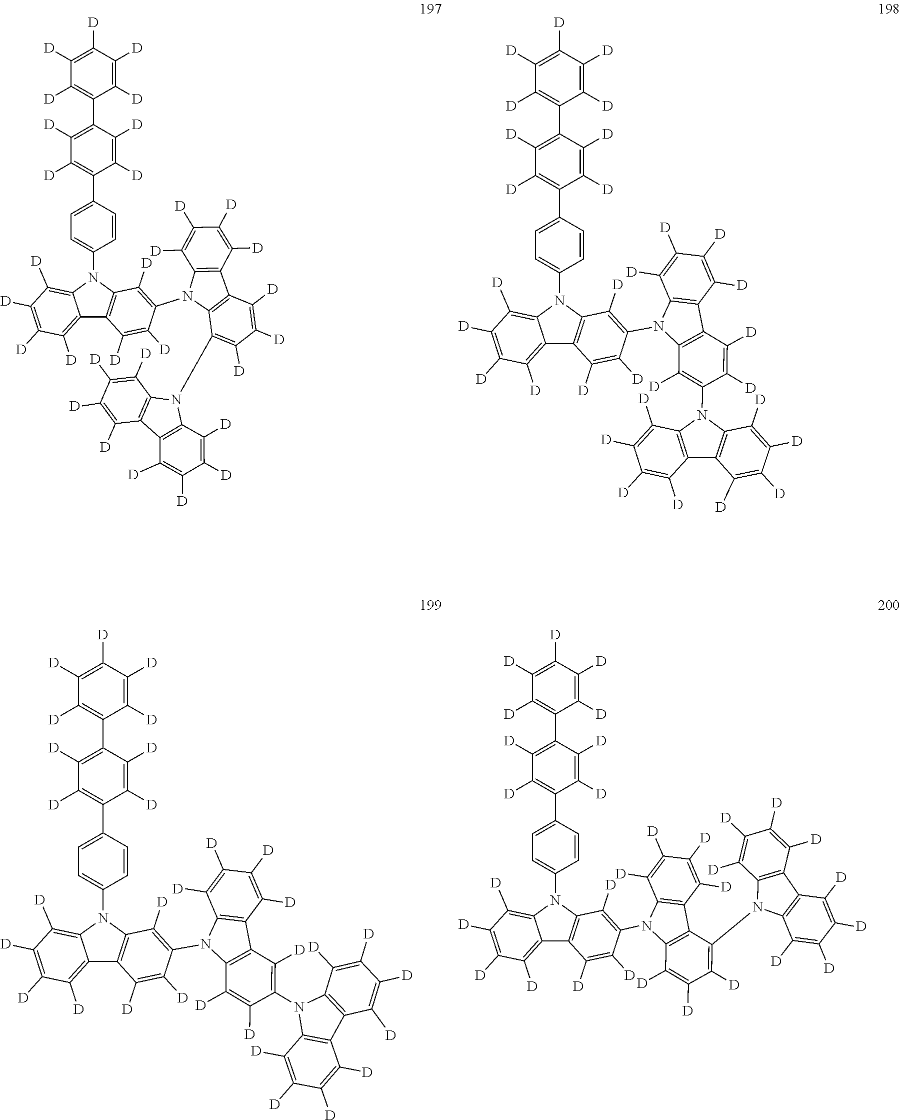

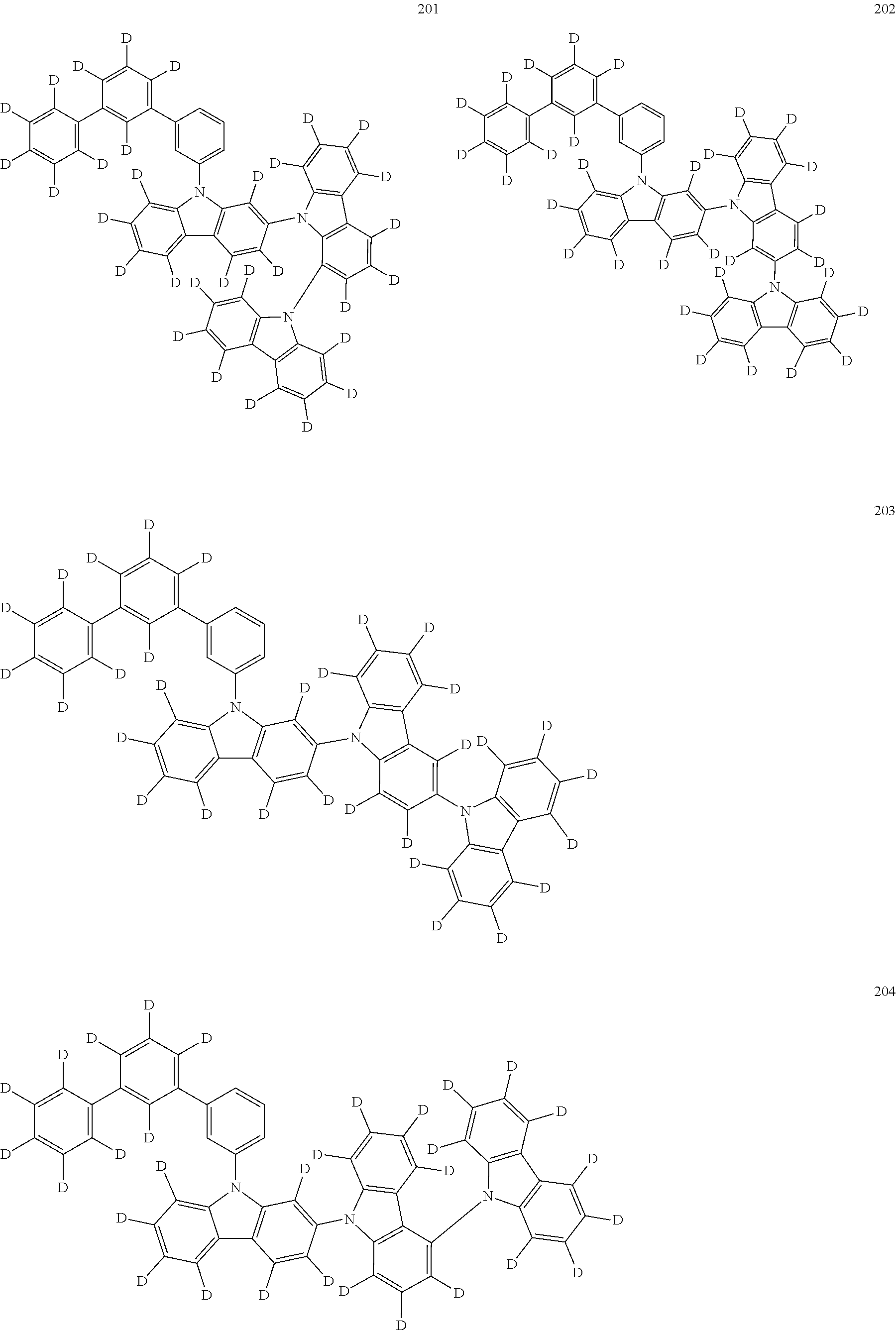

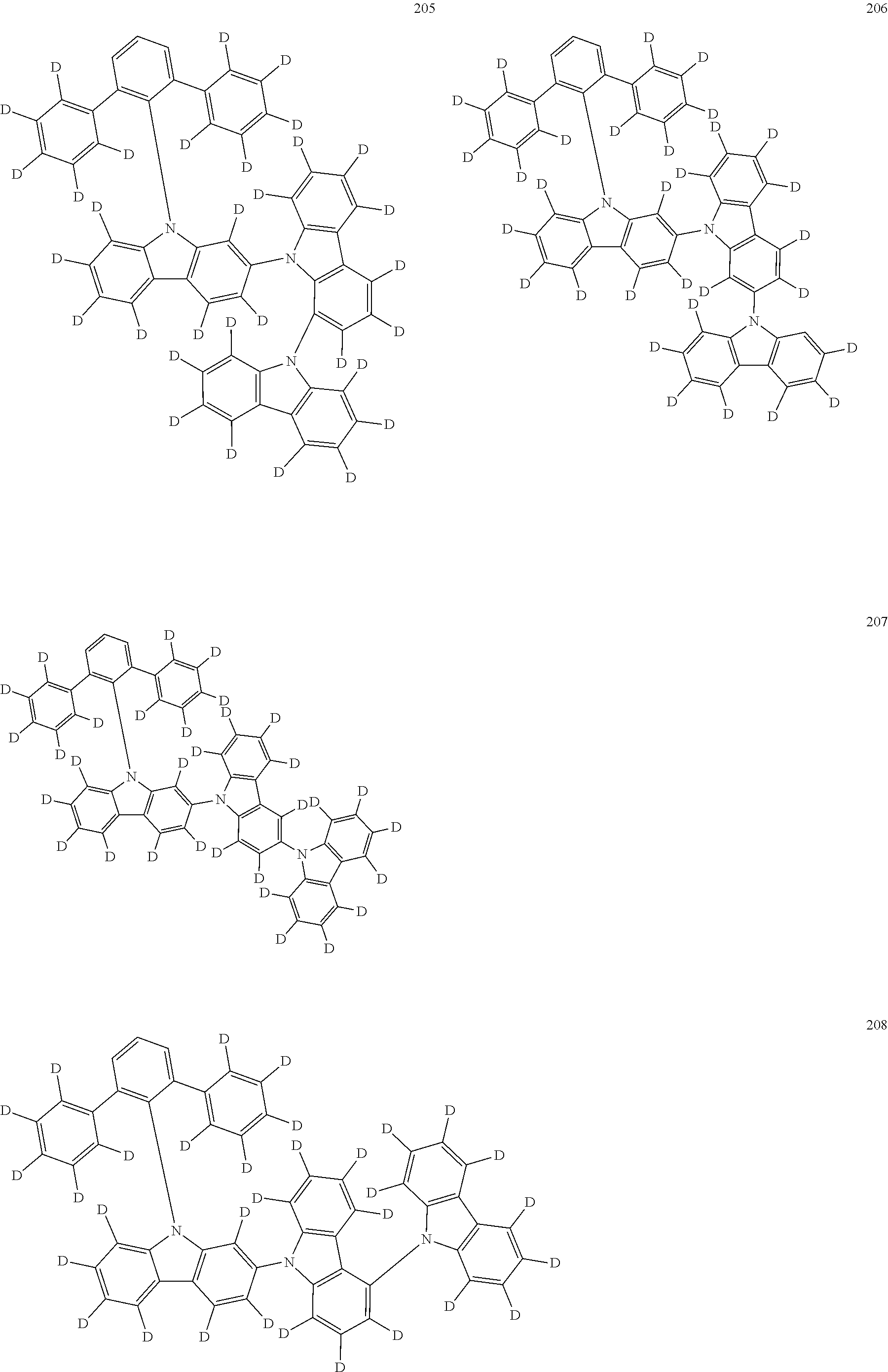

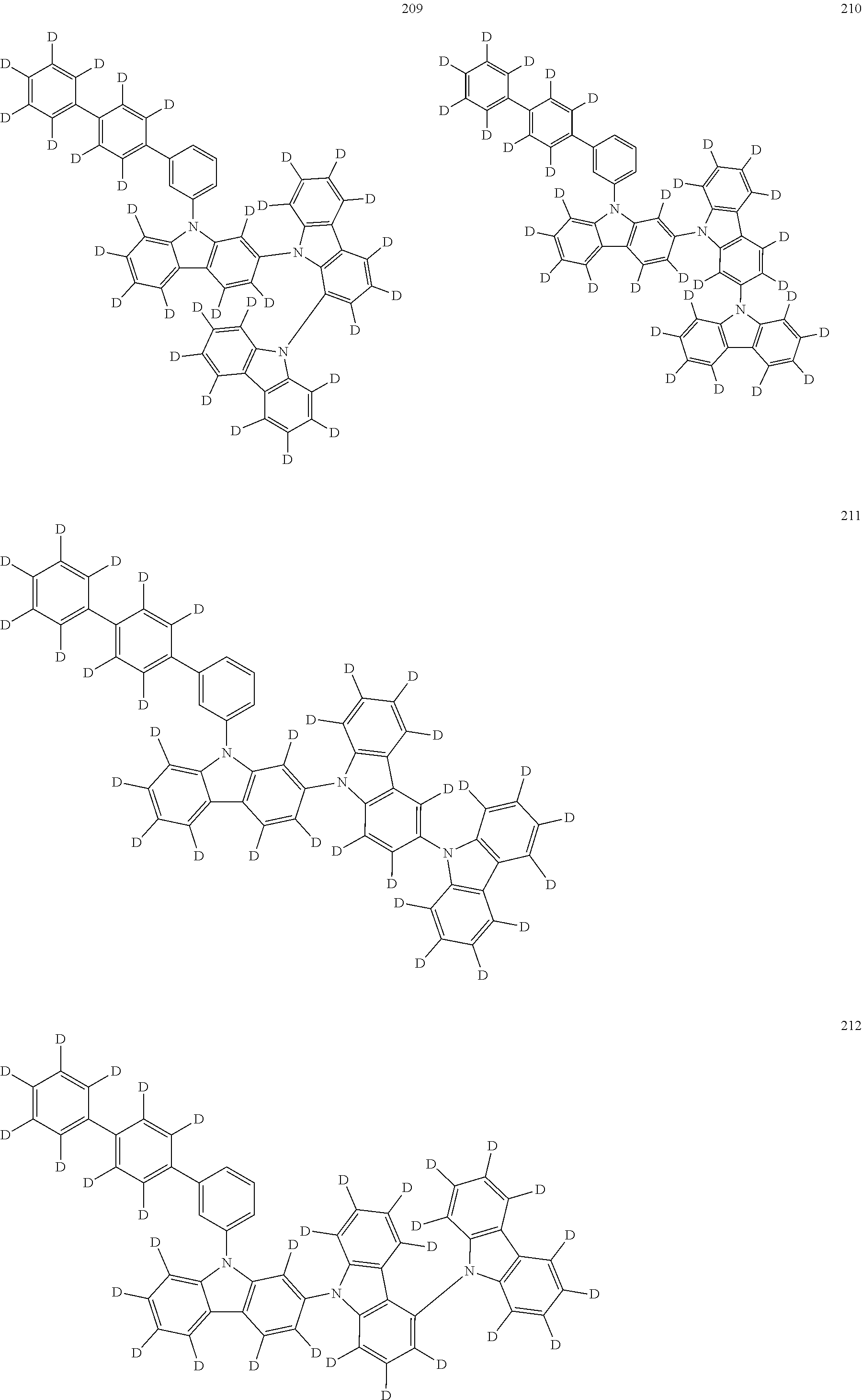

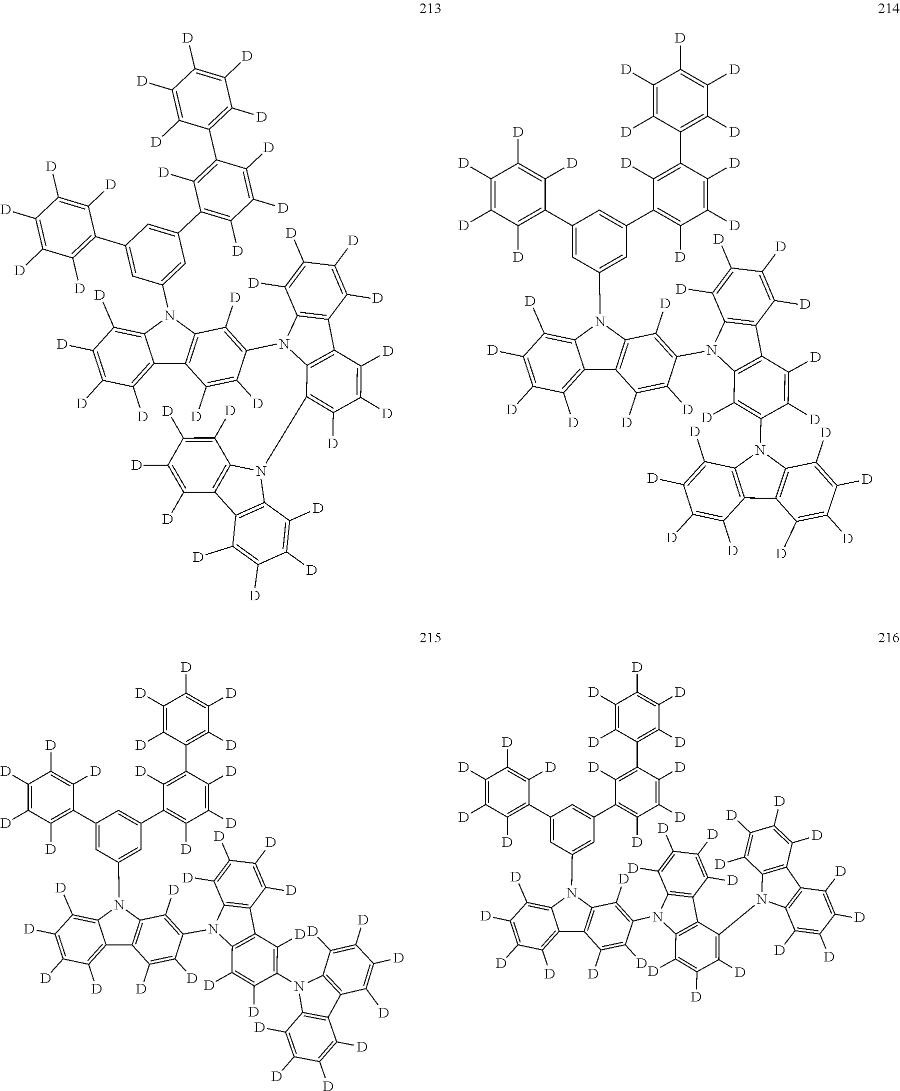

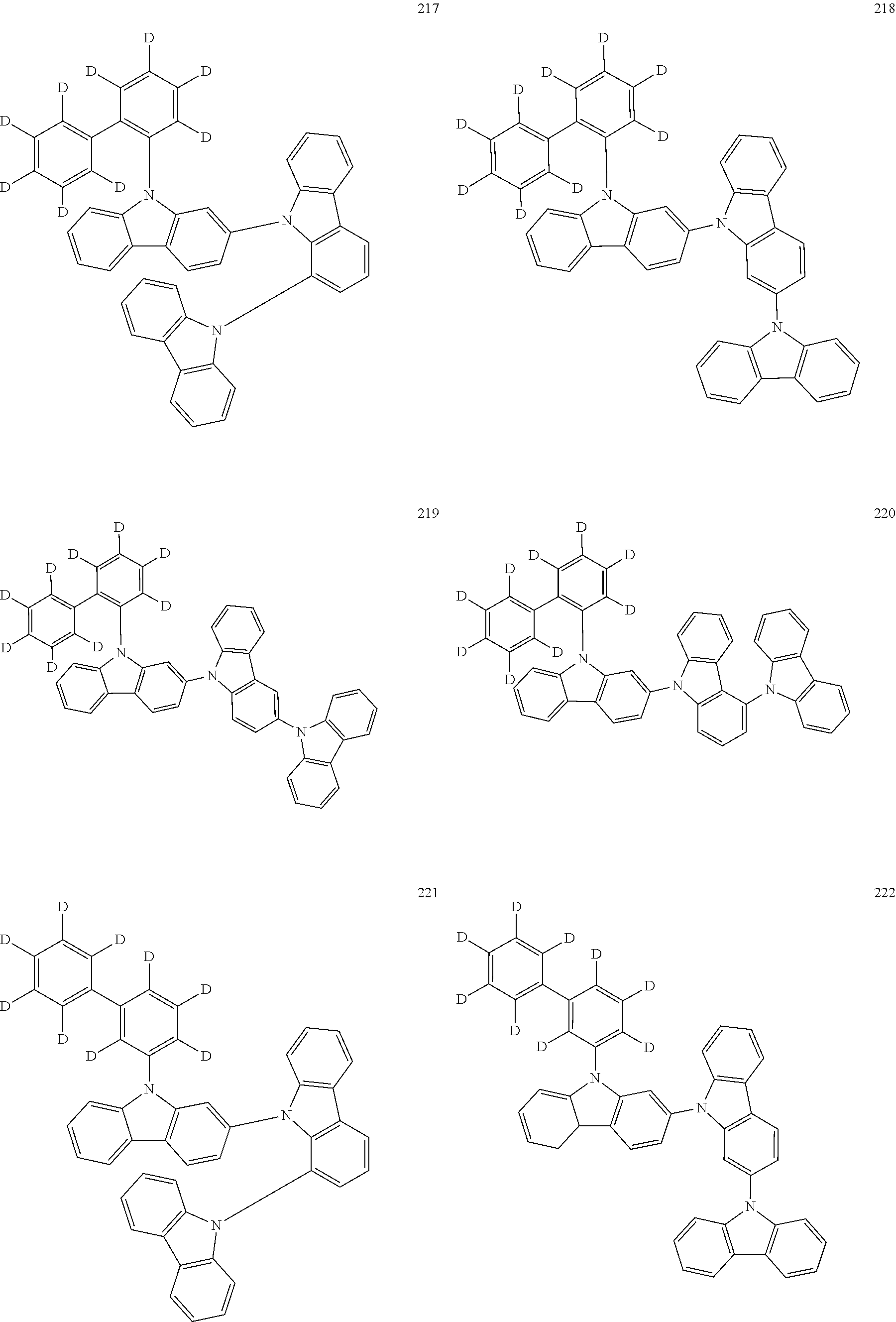

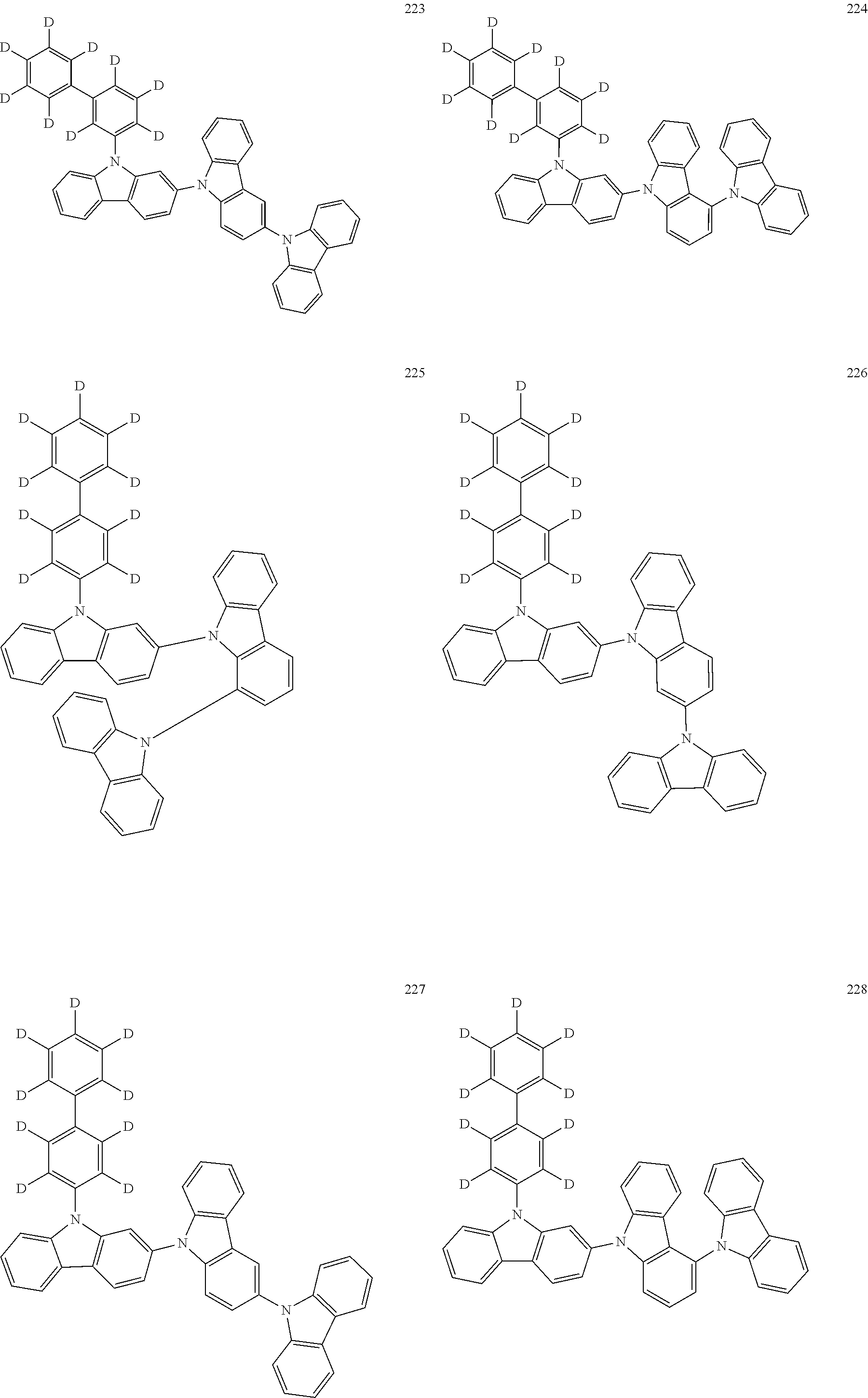

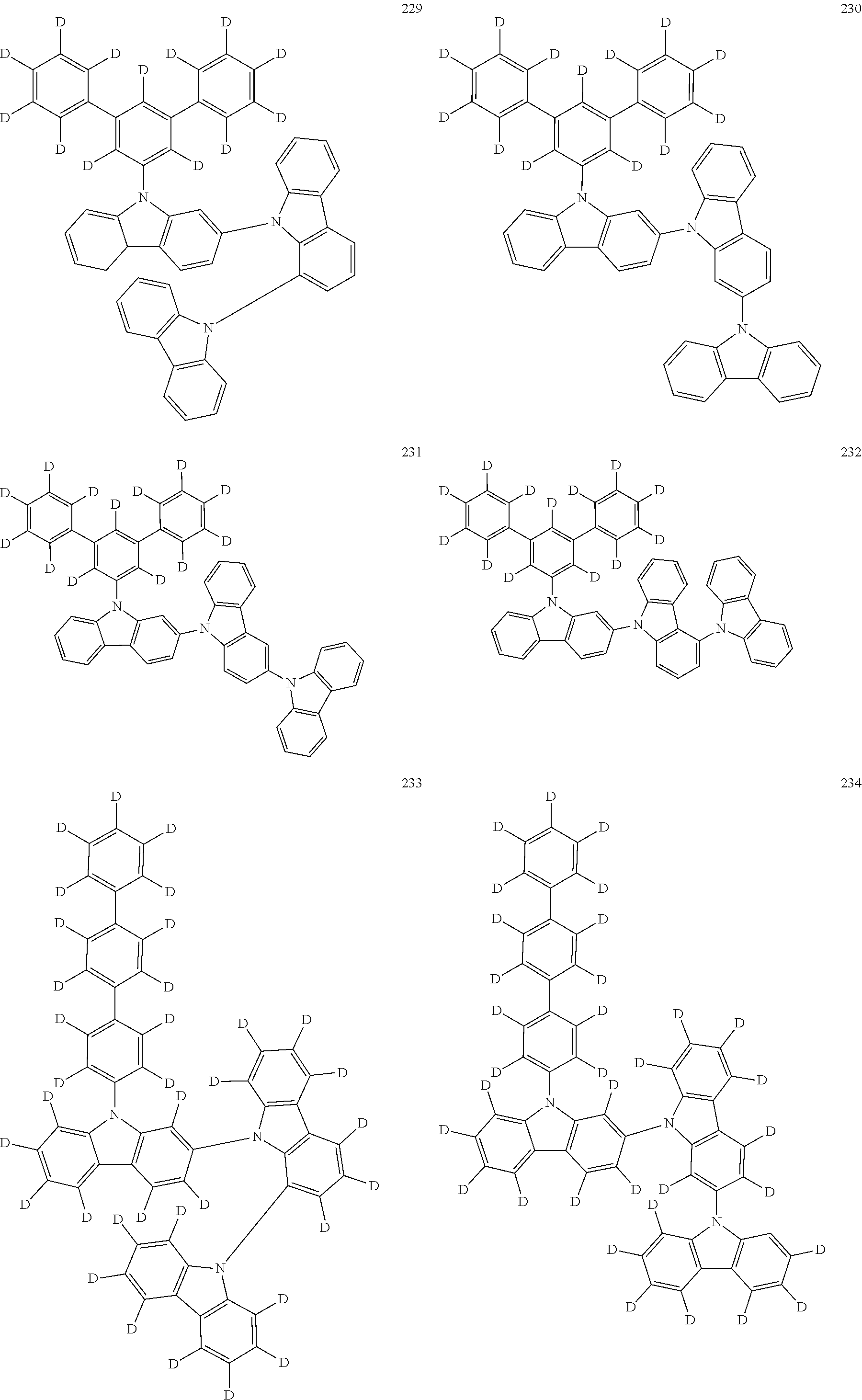

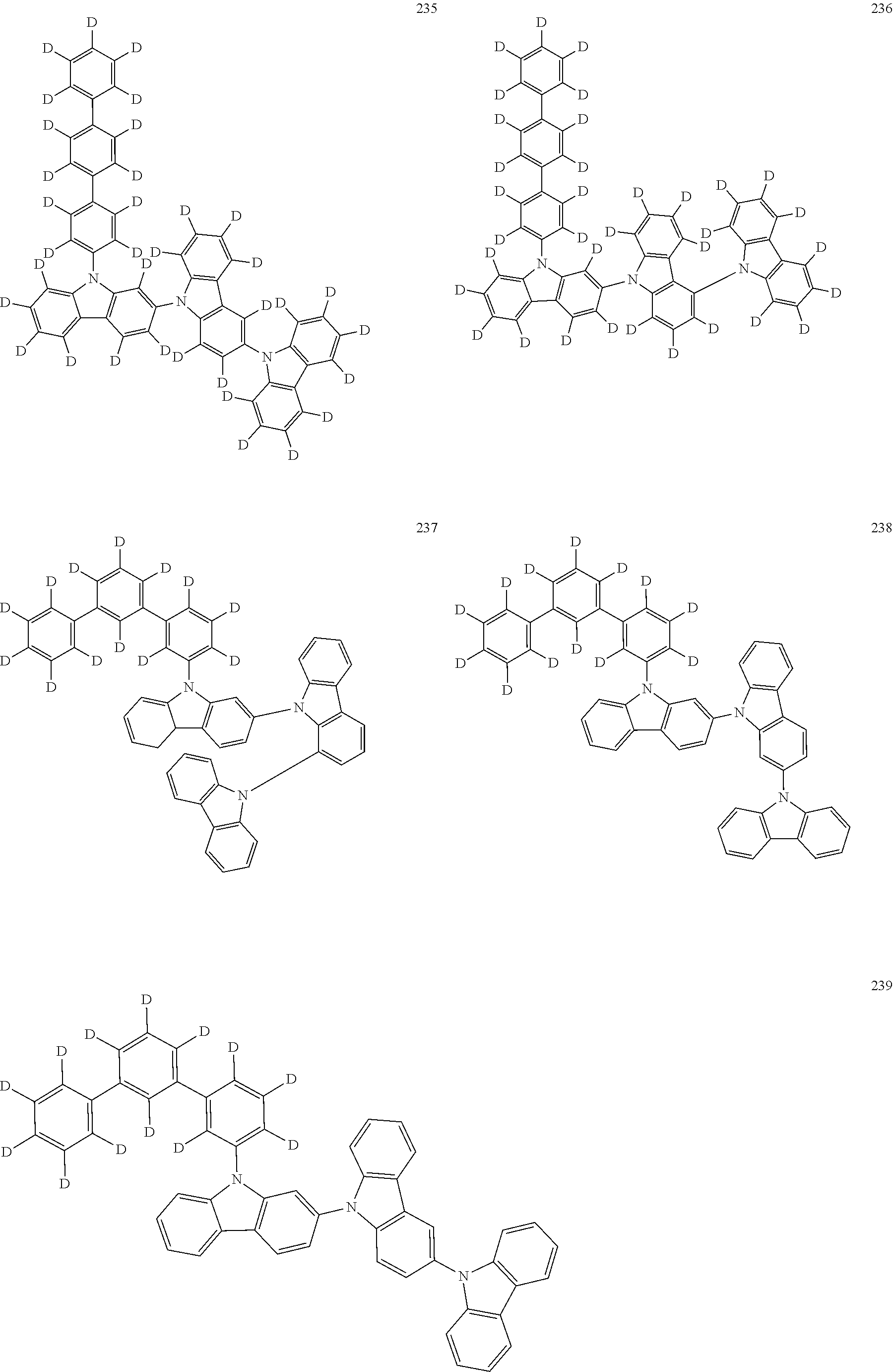

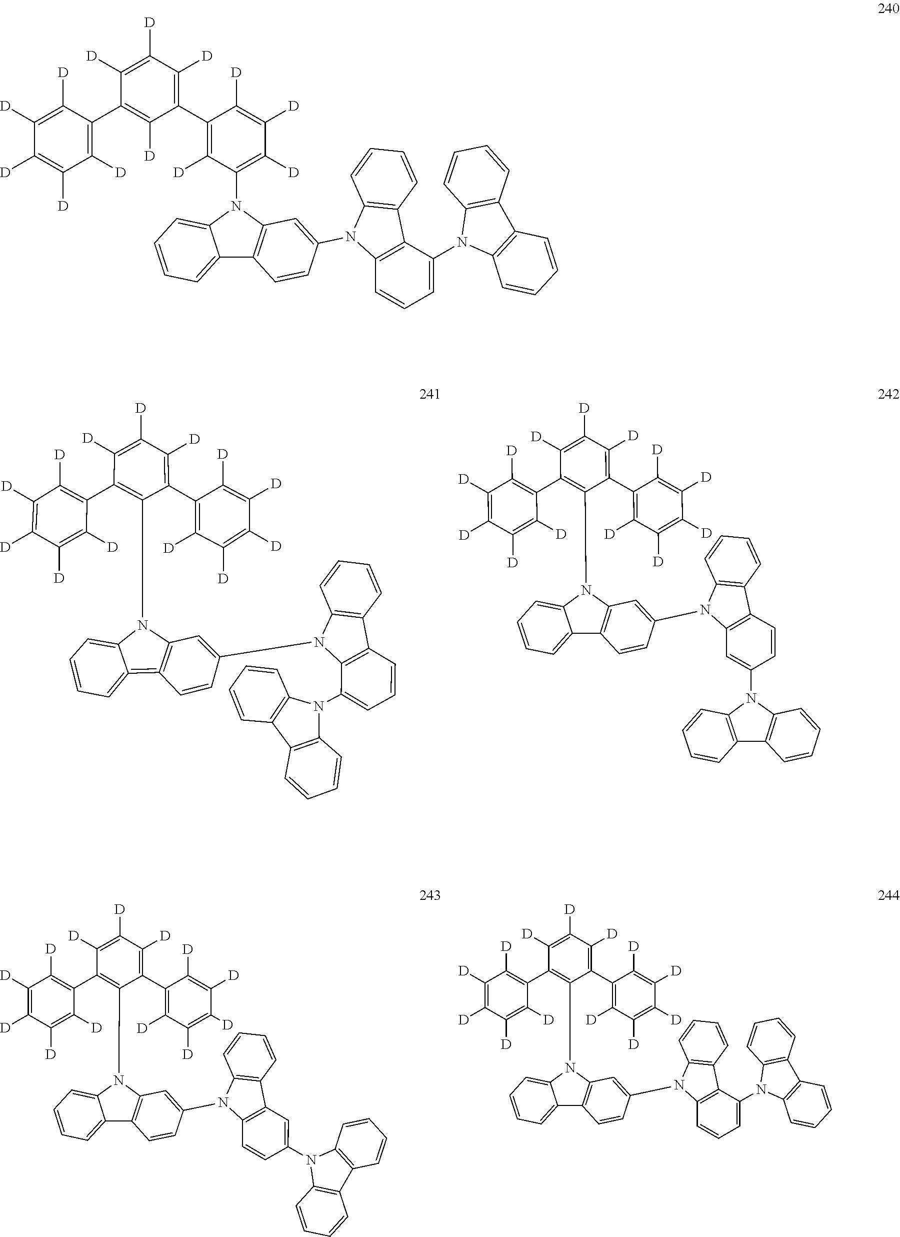

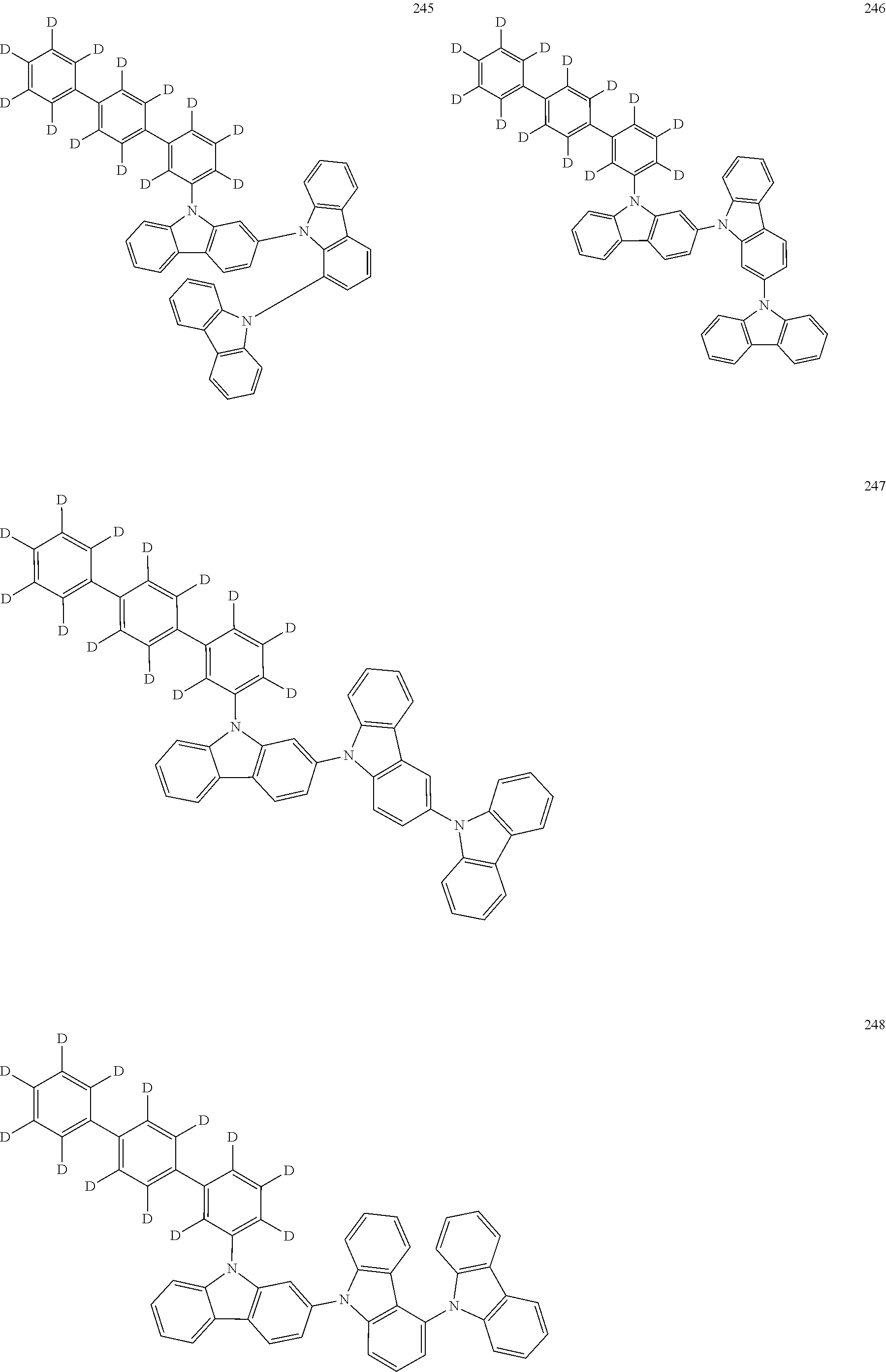

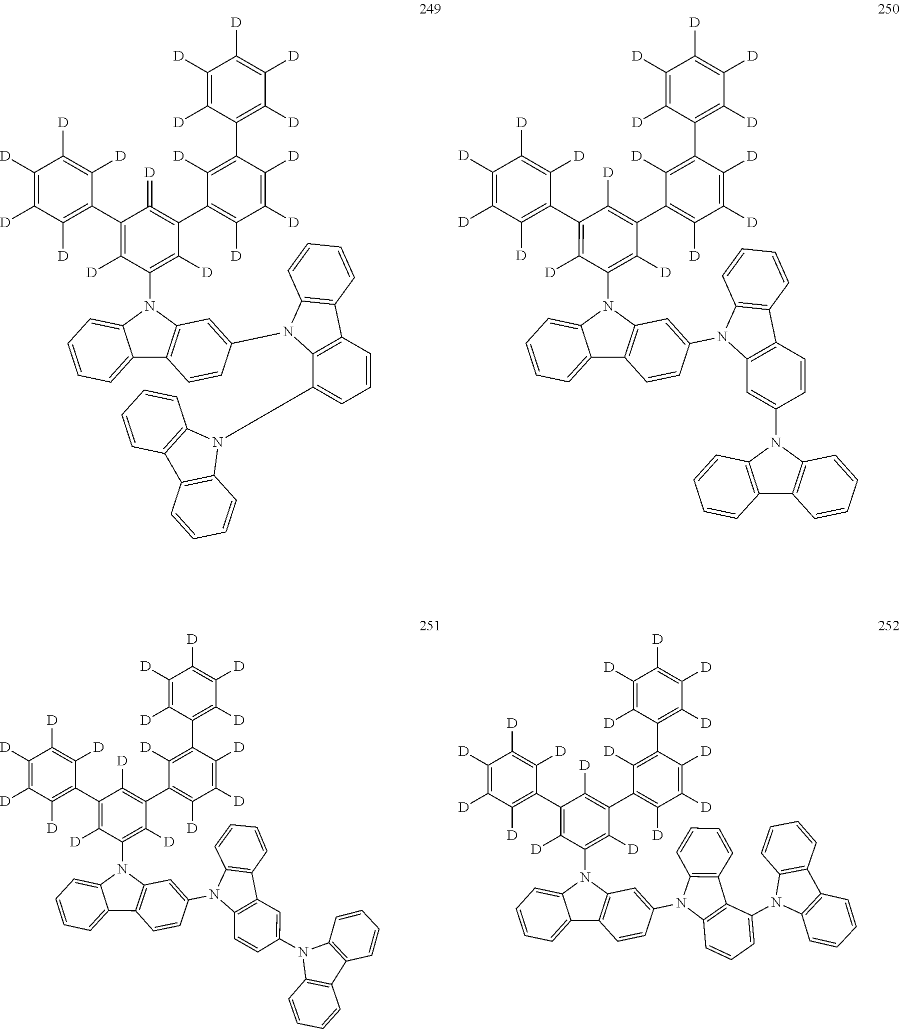

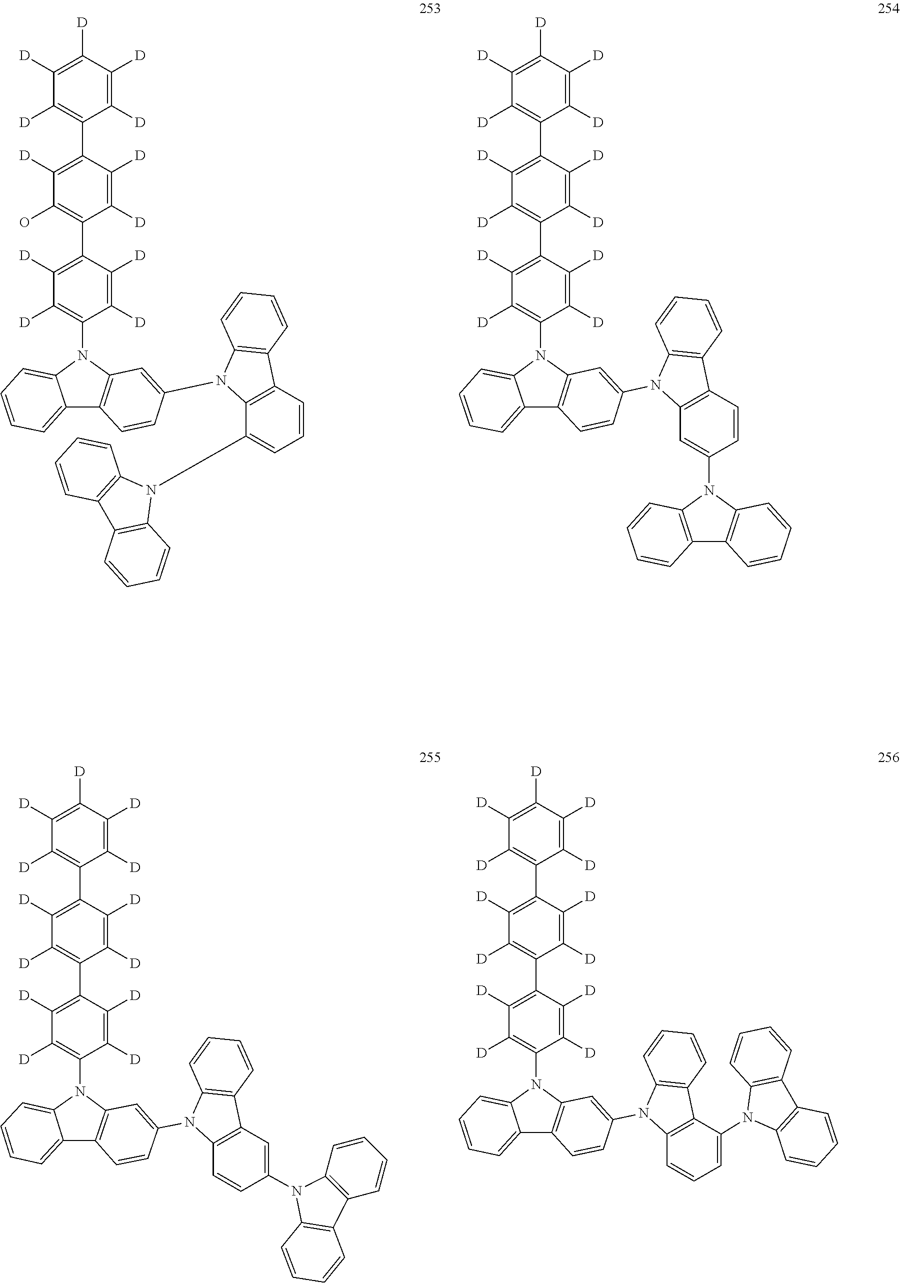

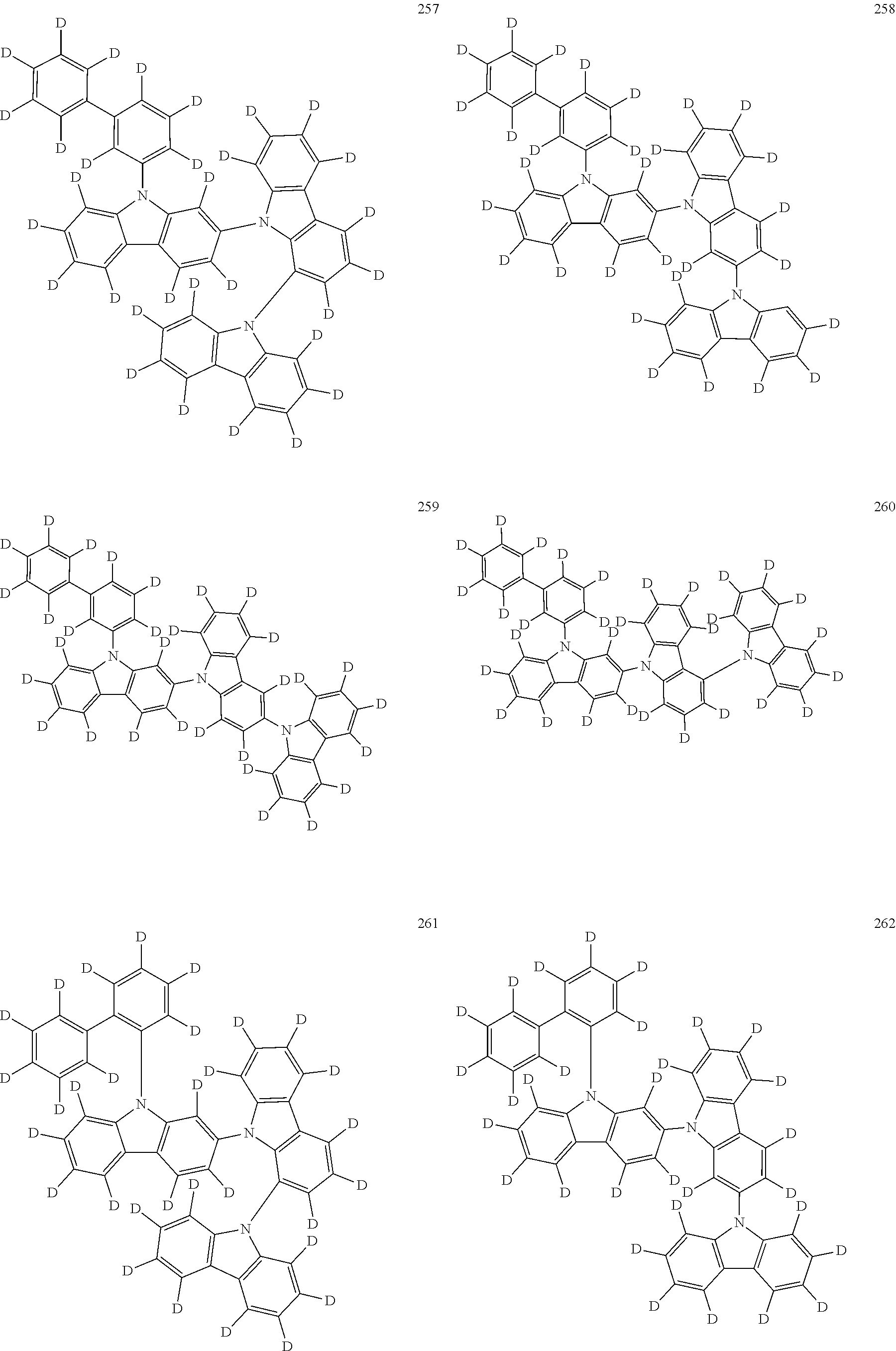

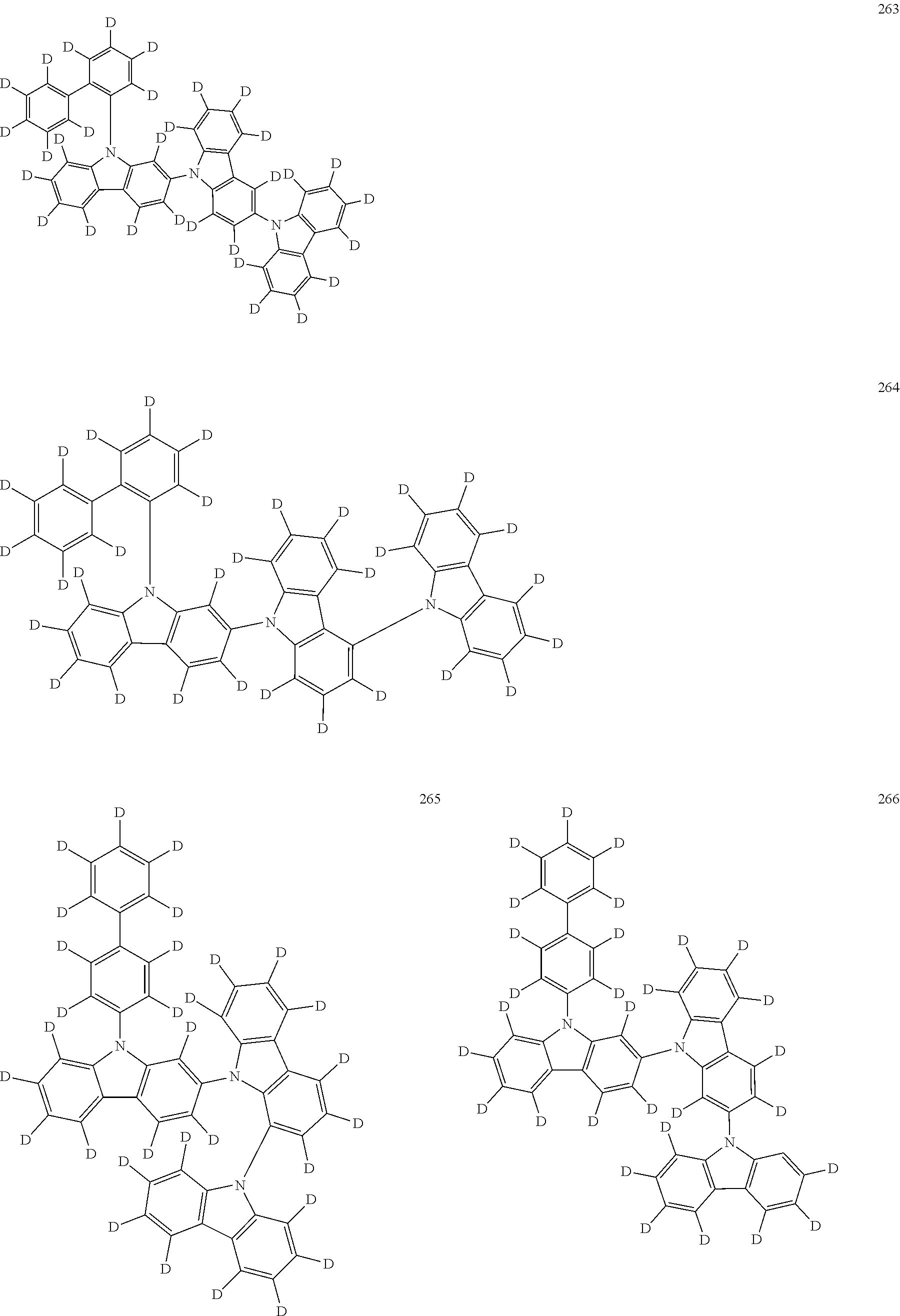

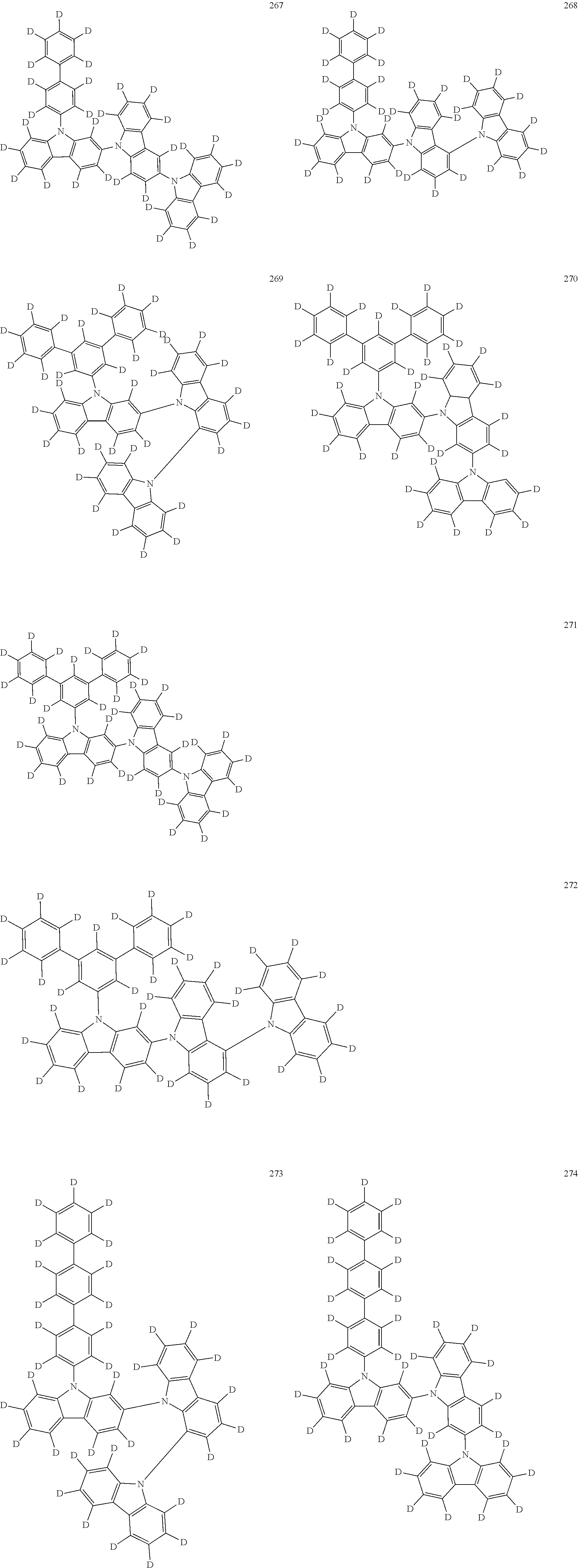

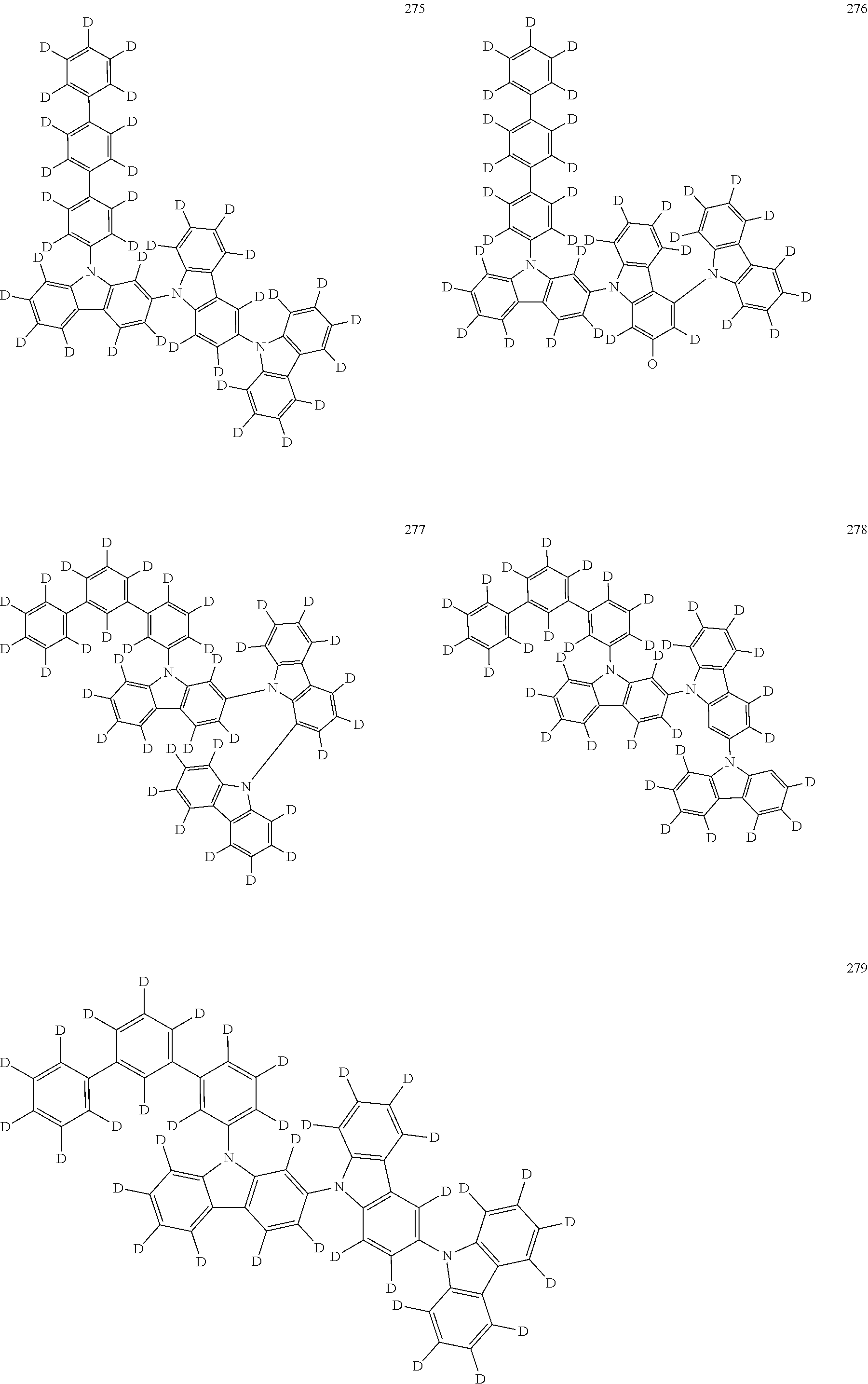

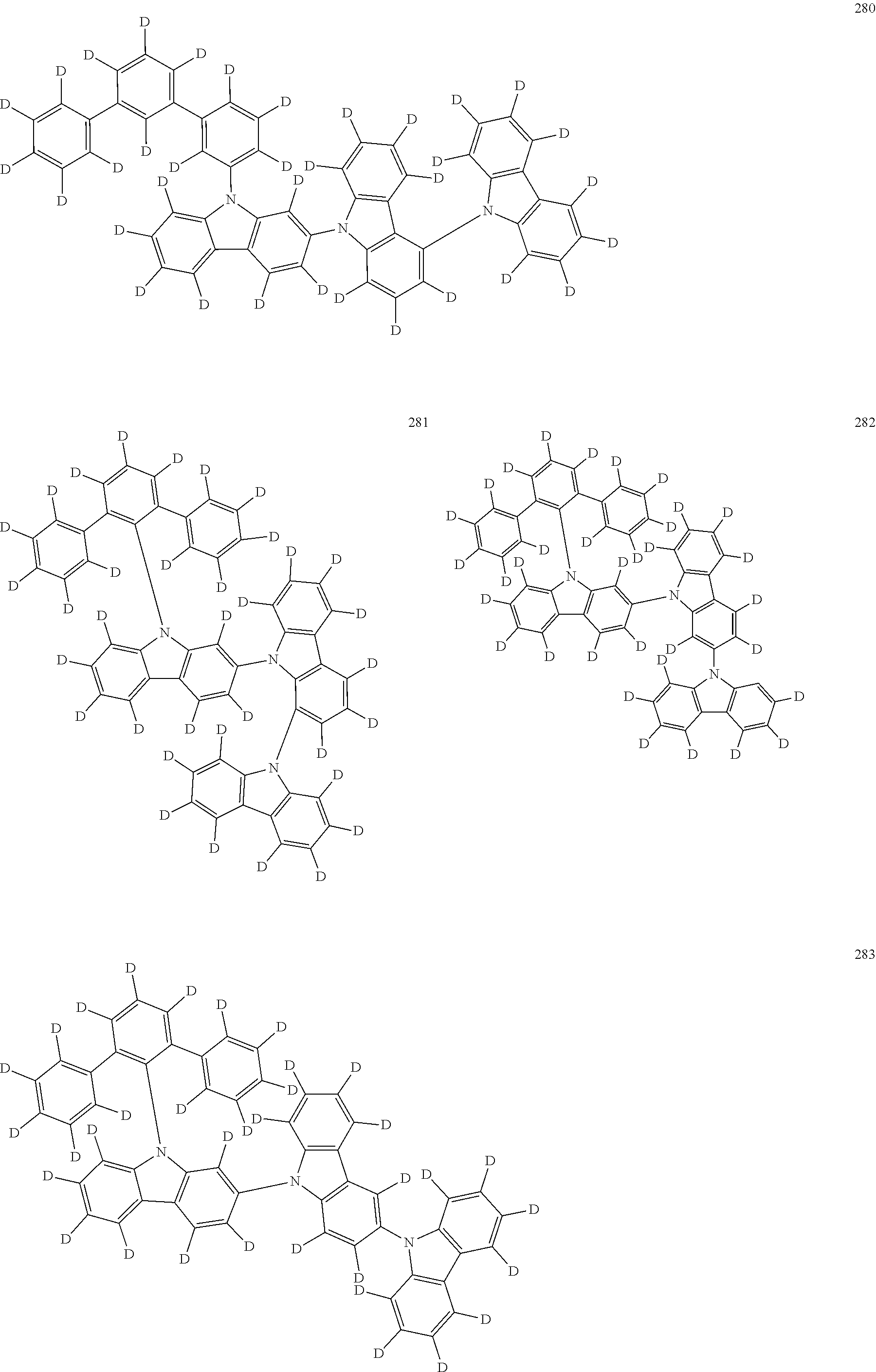

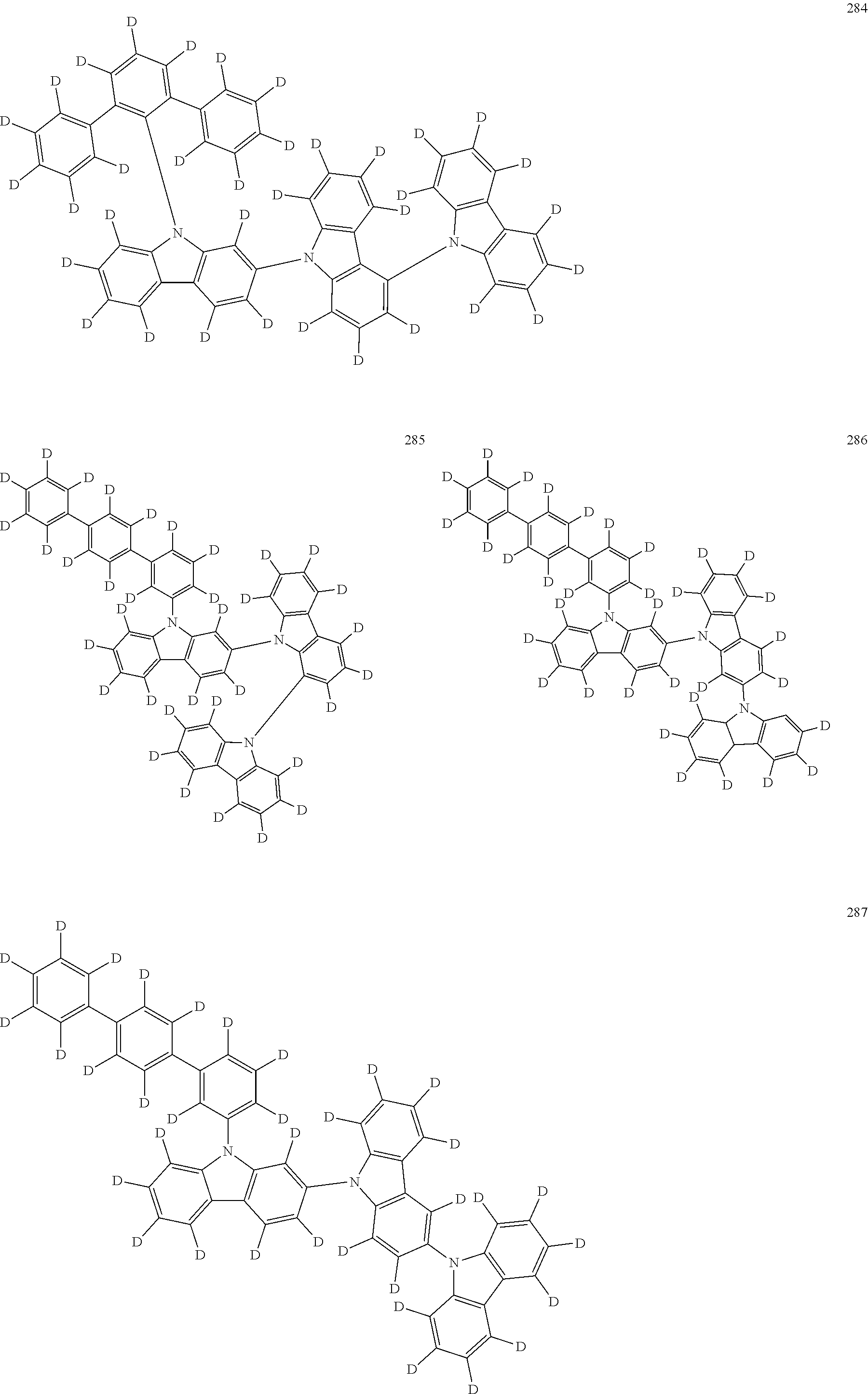

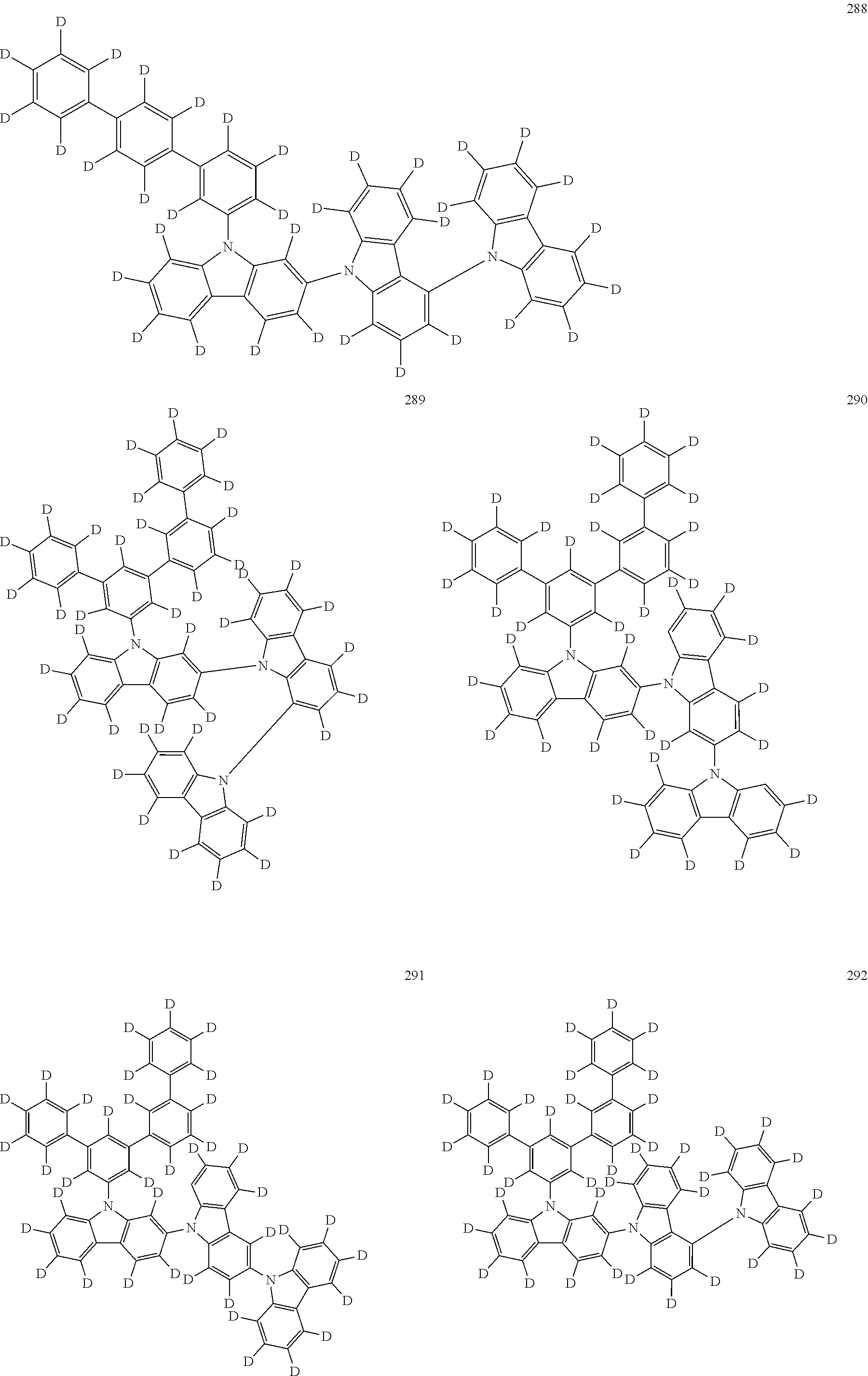

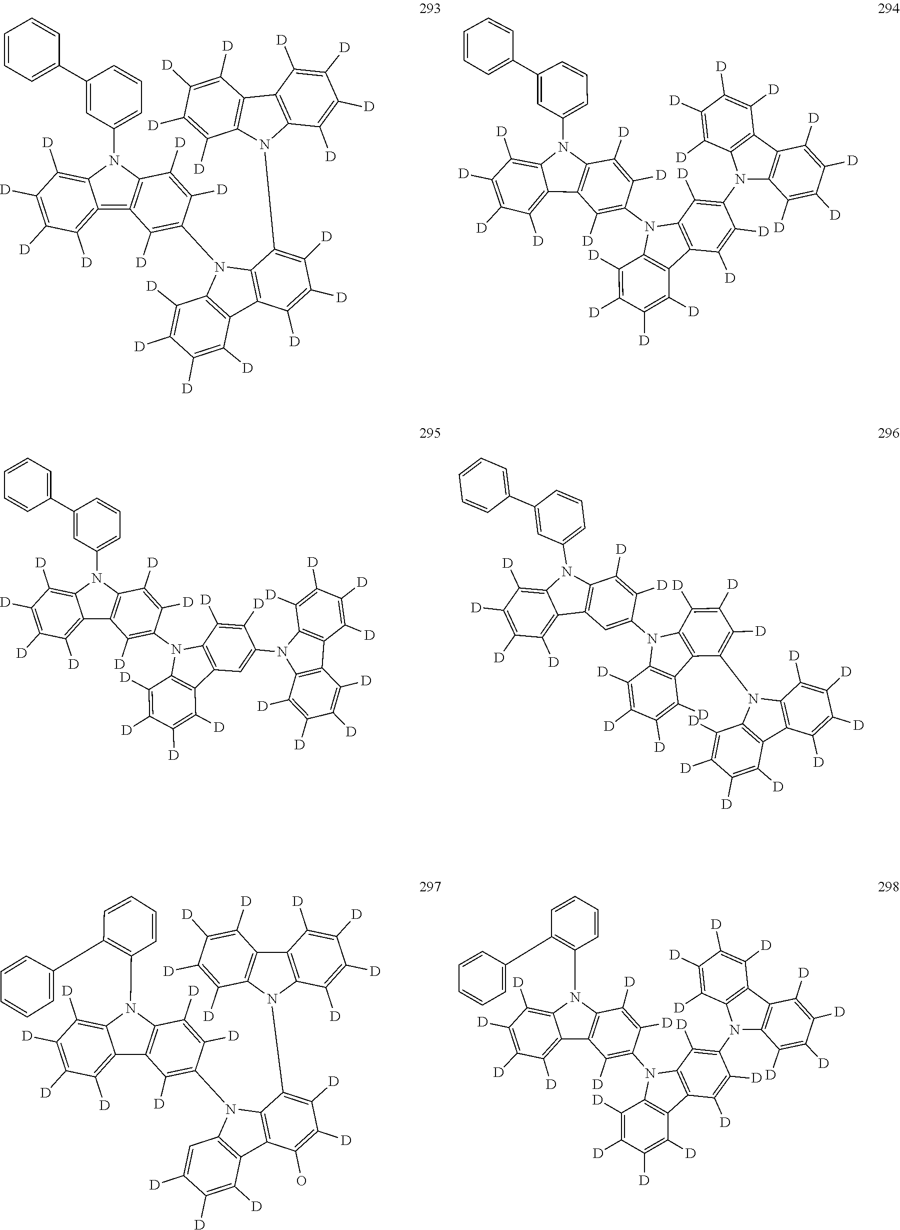

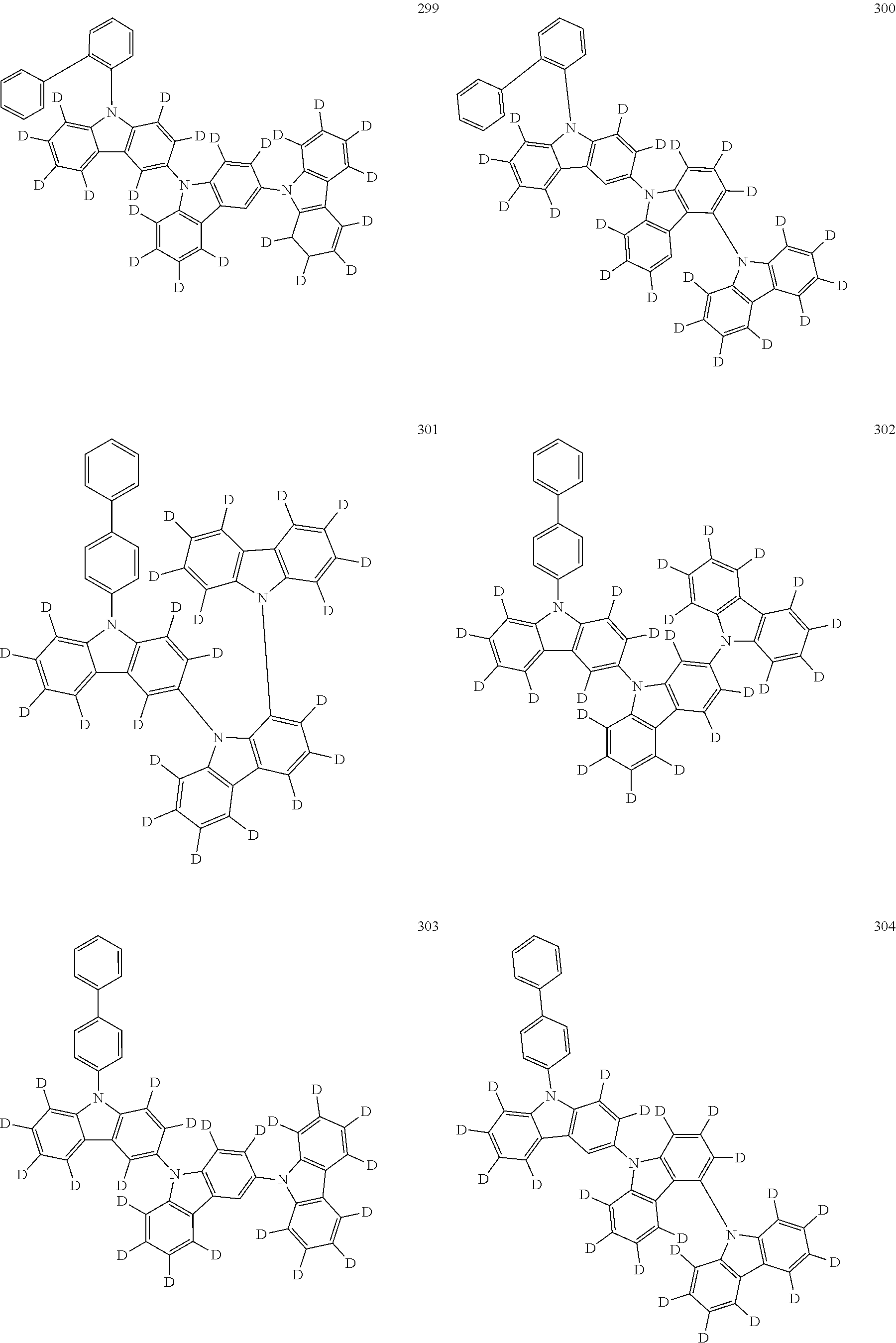

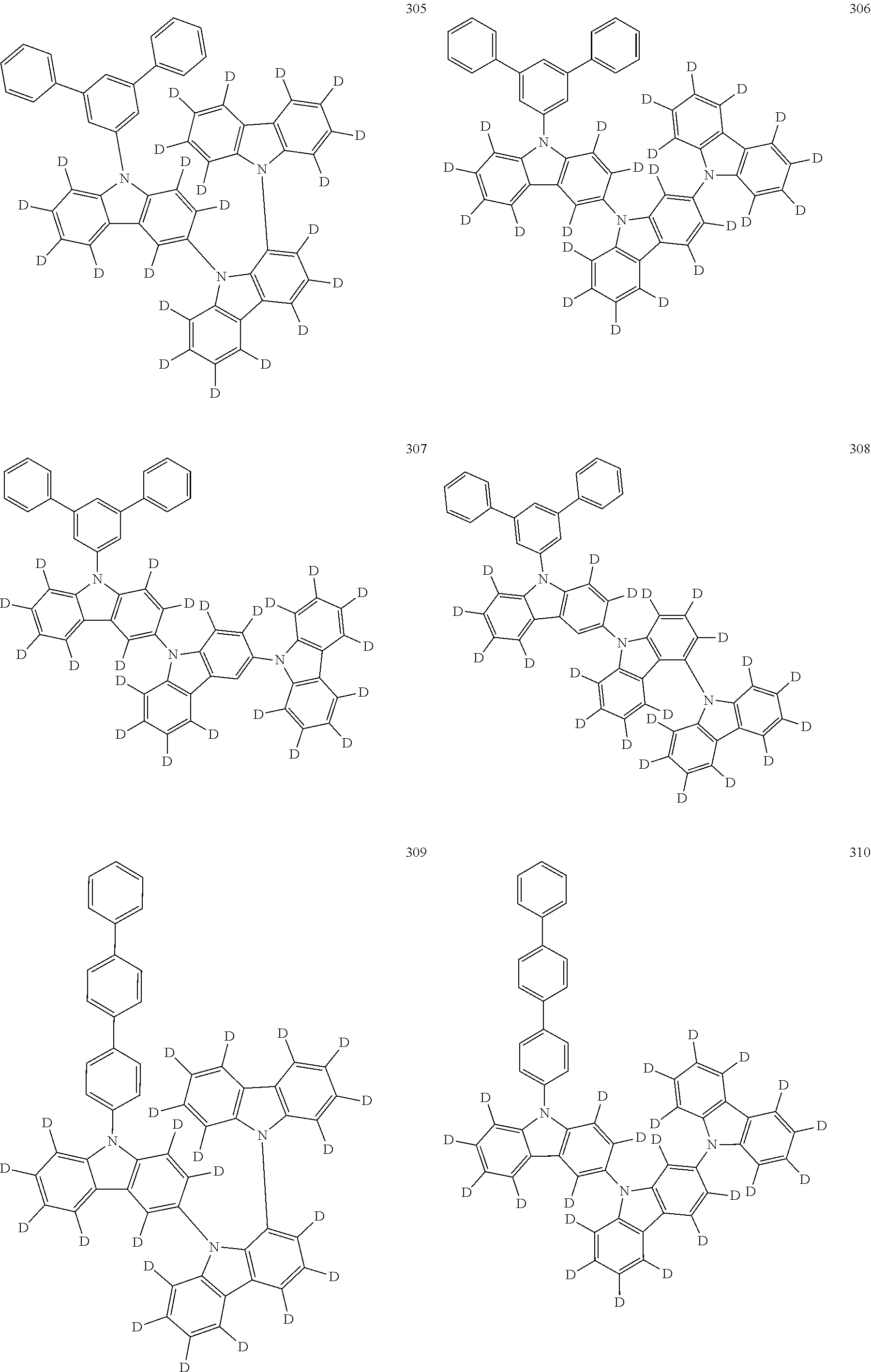

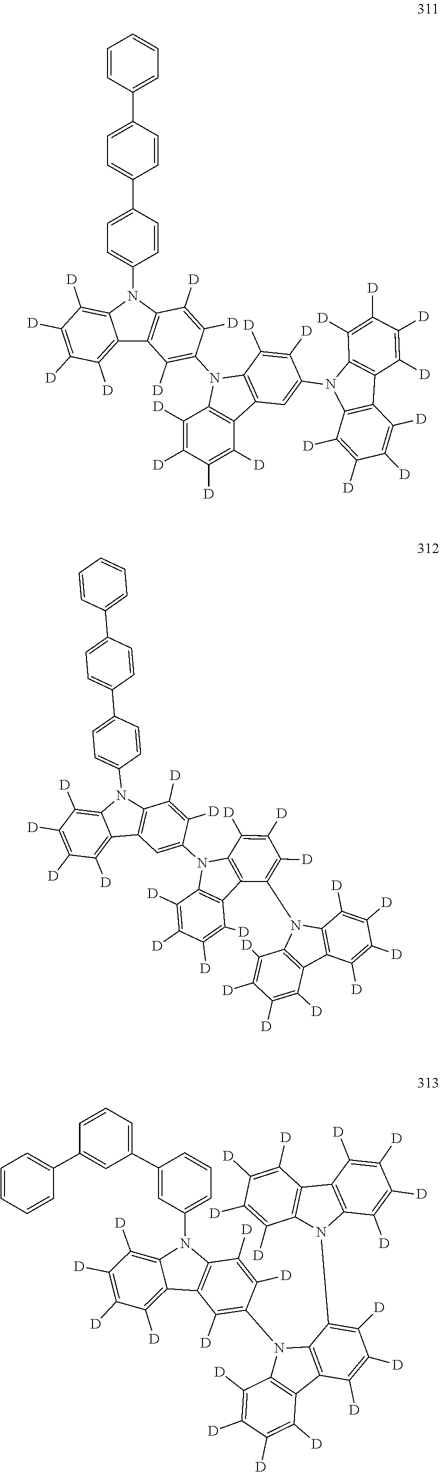

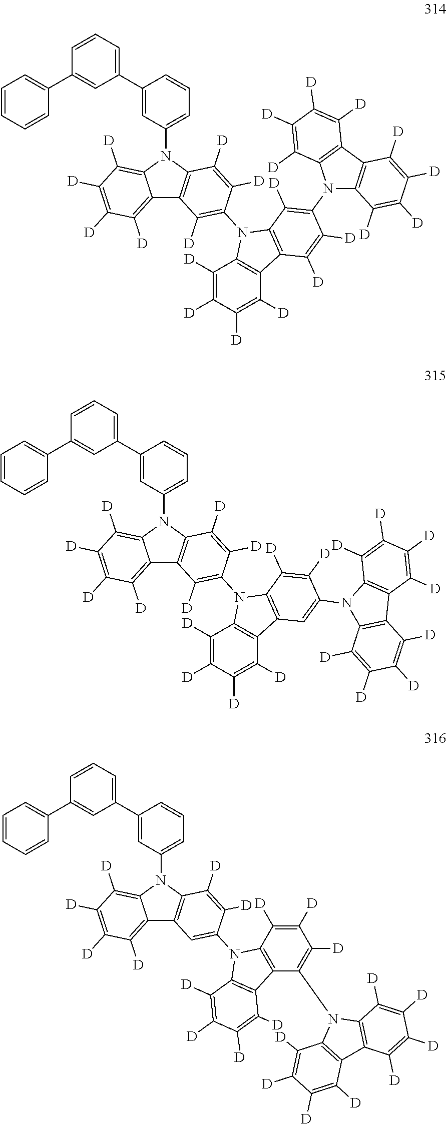

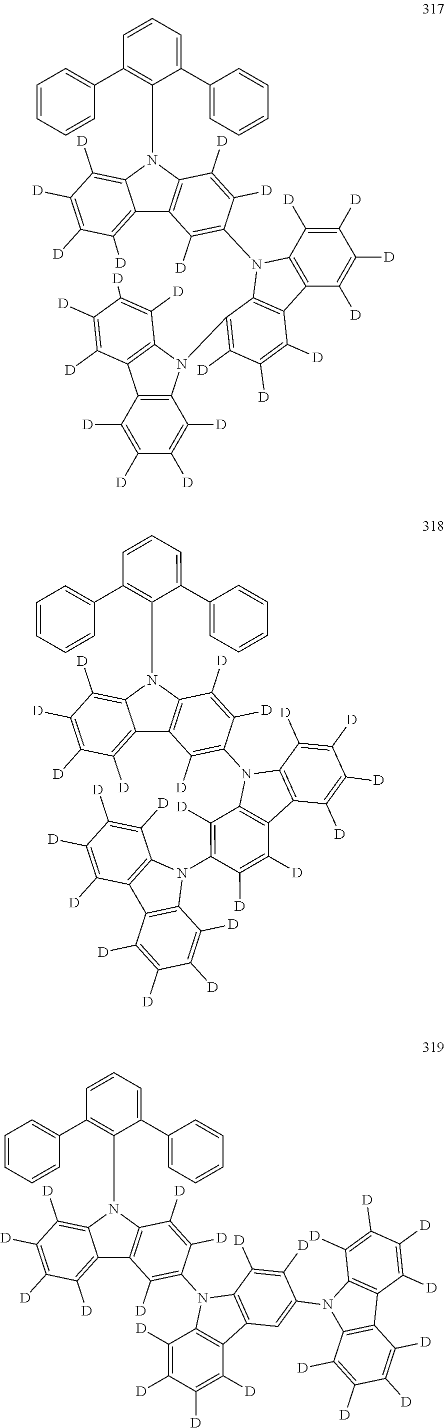

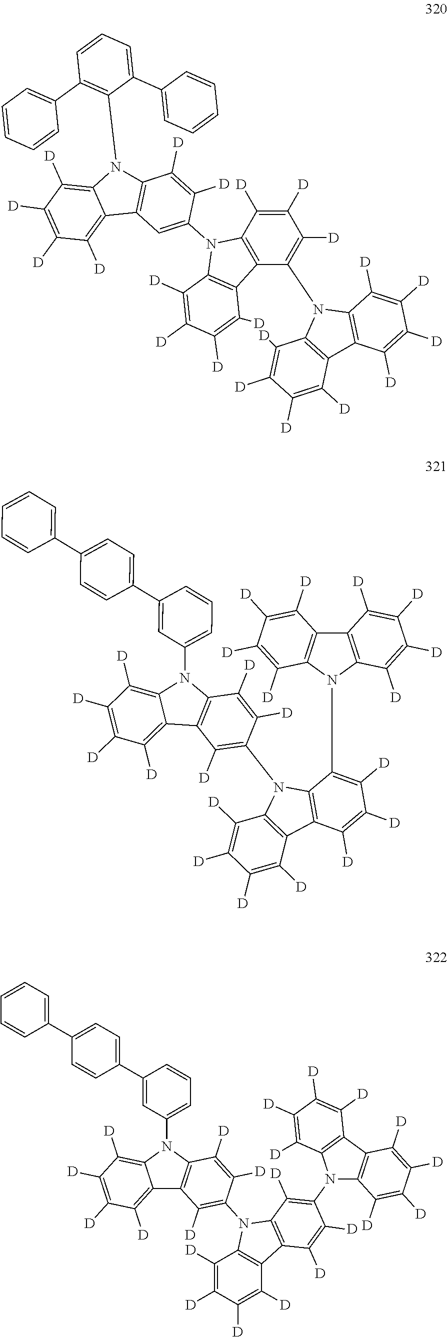

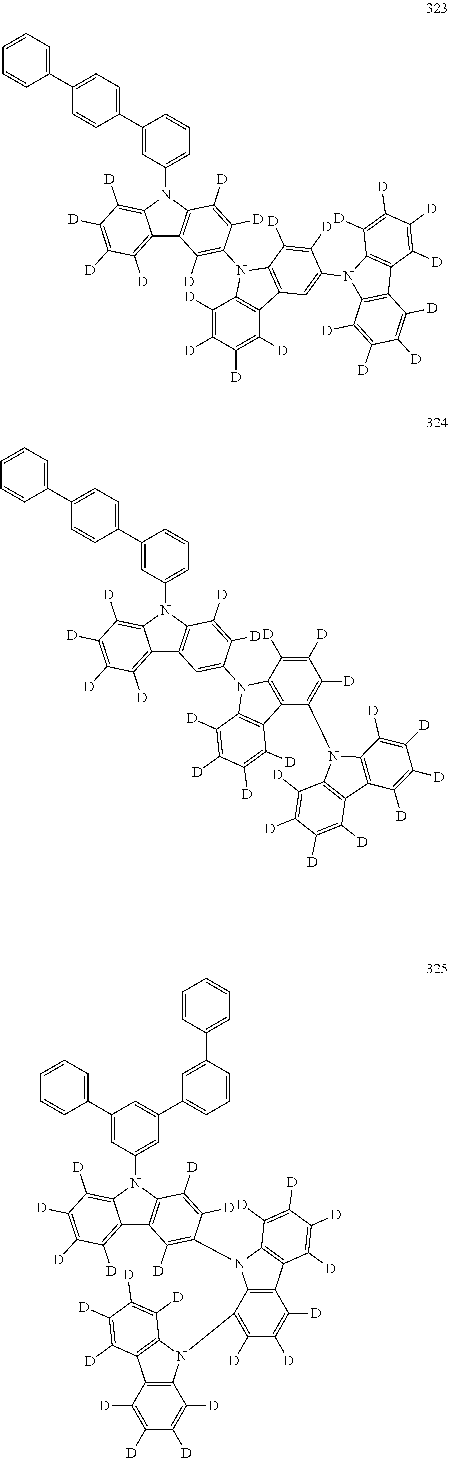

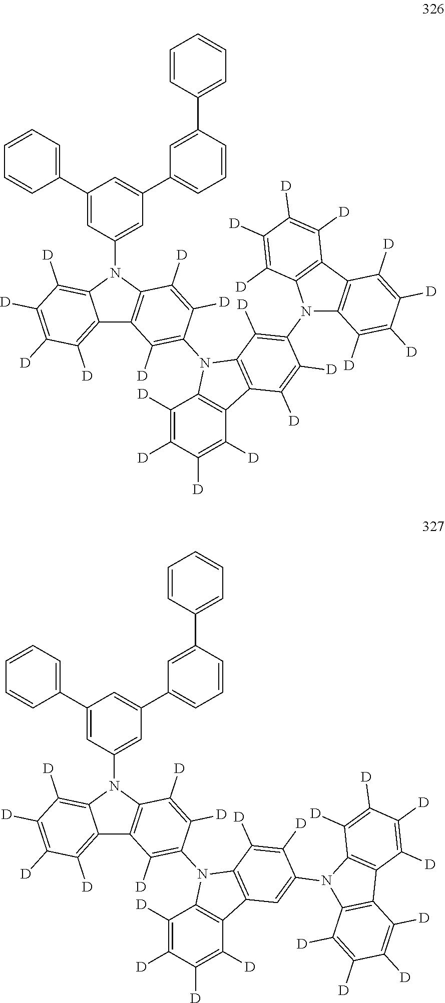 [SMILES: [2H]c1c([2H])c([2H])c2c(c1[2H])c1c([2H])c(-n3c4c([2H])c([2H])c([2H])c([2H])c4c4c([2H])c([2H])c(-n5c6c([2H])c([2H])c([2H])c([2H])c6c6c([2H])c([2H])c([2H])c([2H])c65)c([2H])c43)c([2H])c([2H])c1n2-c1cc(-c2ccccc2)cc(-c2cccc(-c3ccccc3)c2)c1 ; [2H]c1c([2H])c([2H])c2c(c1[2H])c1cc(-n3c4c([2H])c([2H])c([2H])c([2H])c4c4c([2H])c([2H])c([2H])c([2H])c43)c([2H])c([2H])c1n2-c1c([2H])c([2H])c2c(c1[2H])c1c([2H])c([2H])c([2H])c([2H])c1n2-c1cc(-c2ccccc2)cc(-c2cccc(-c3ccccc3)c2)c1]

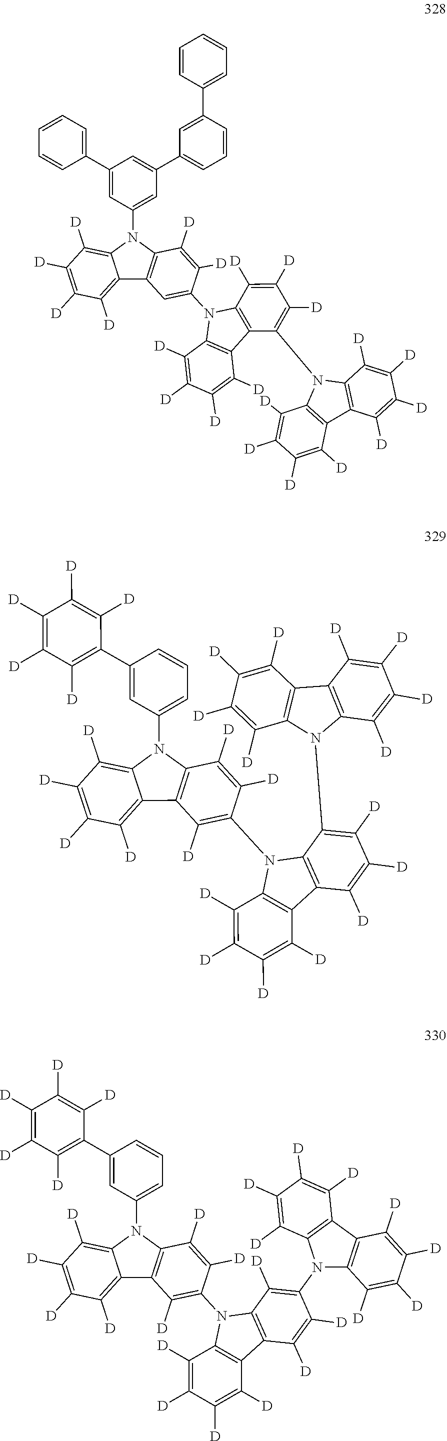

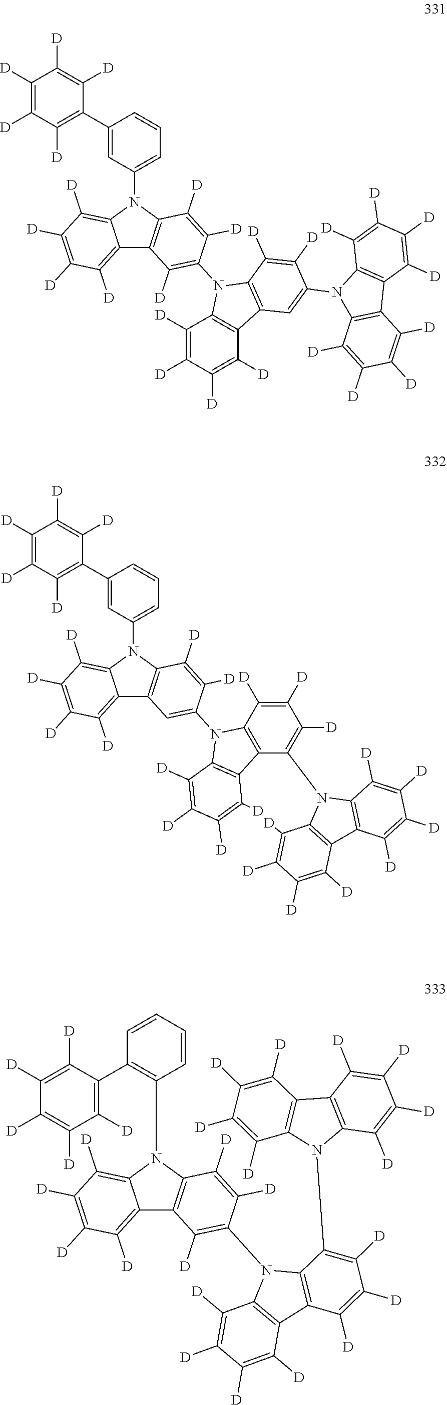

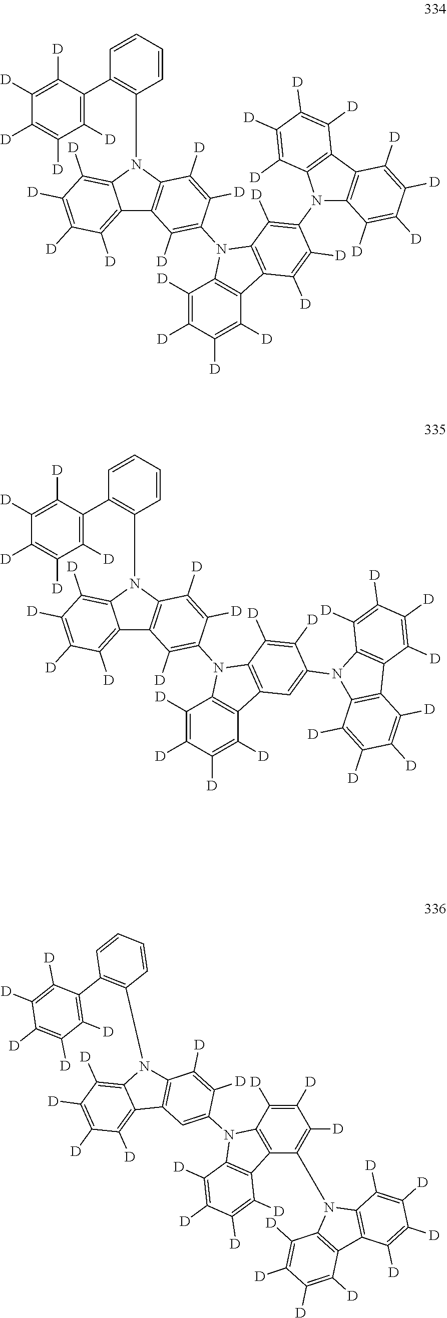

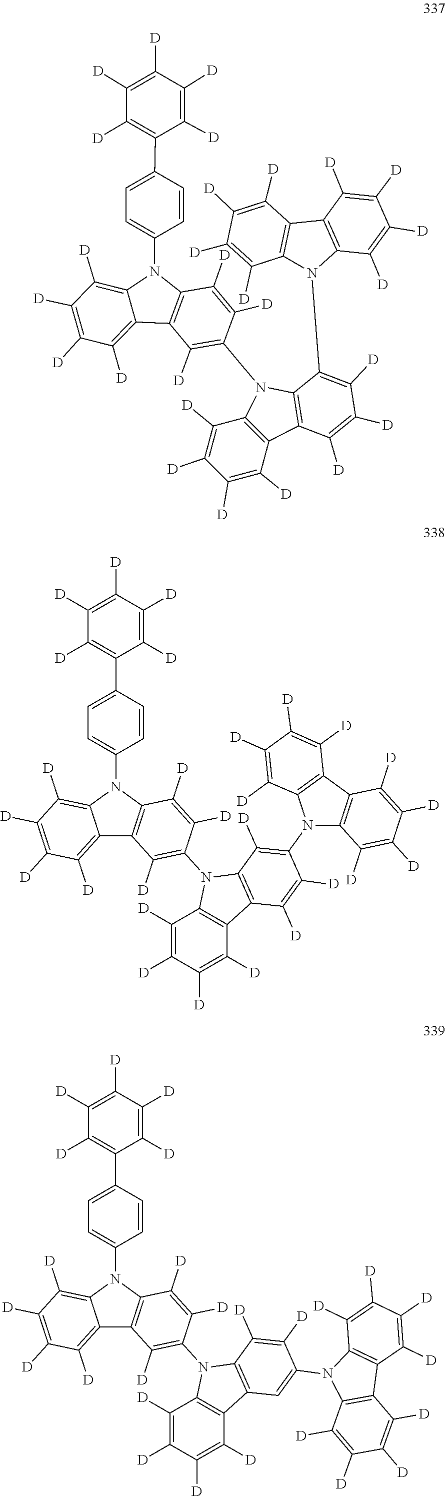

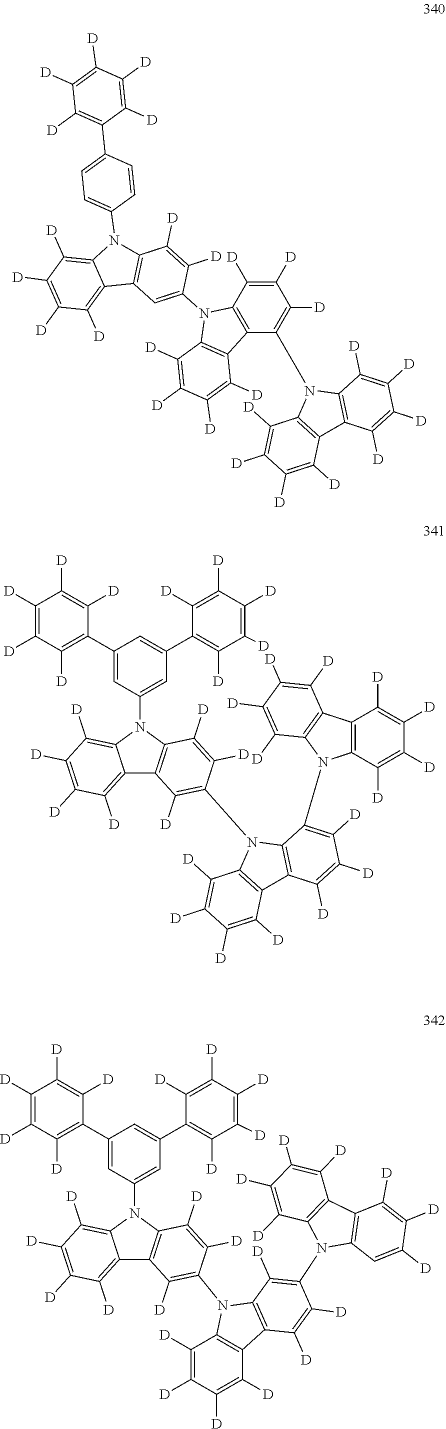

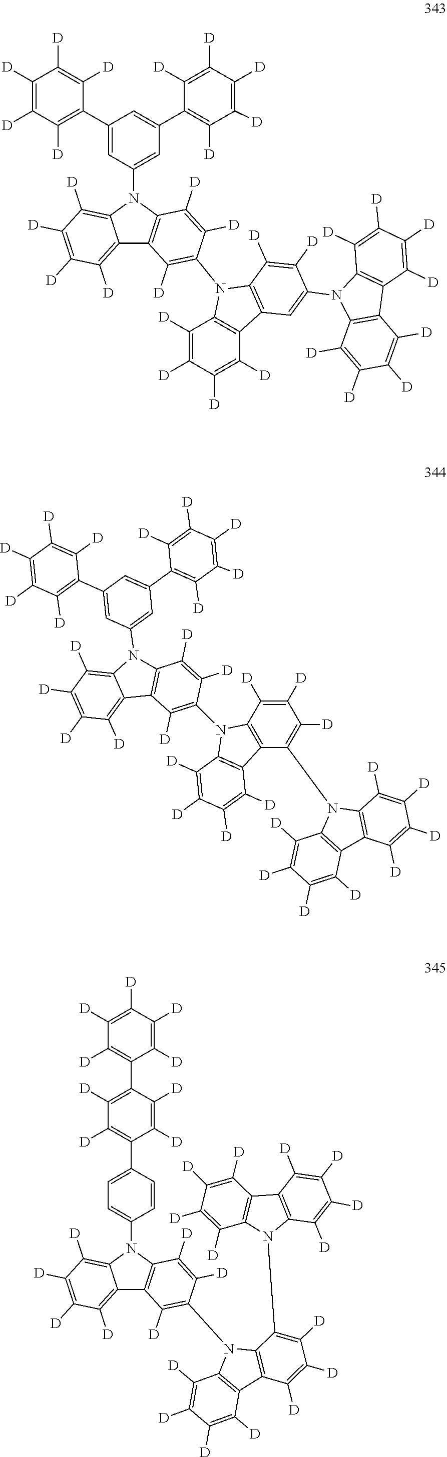

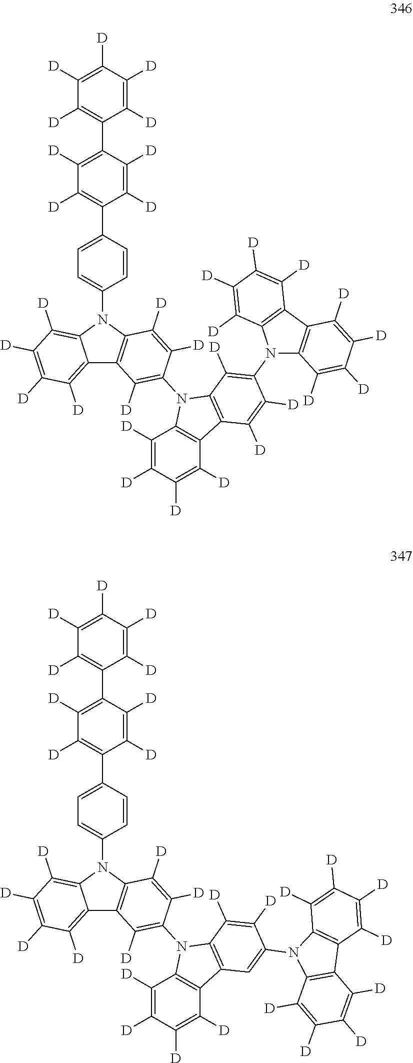

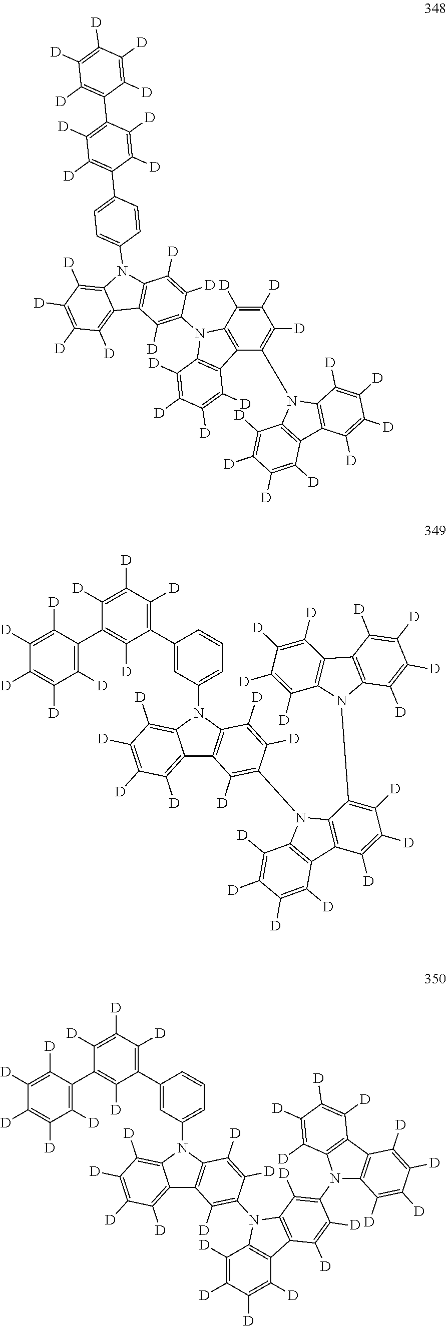

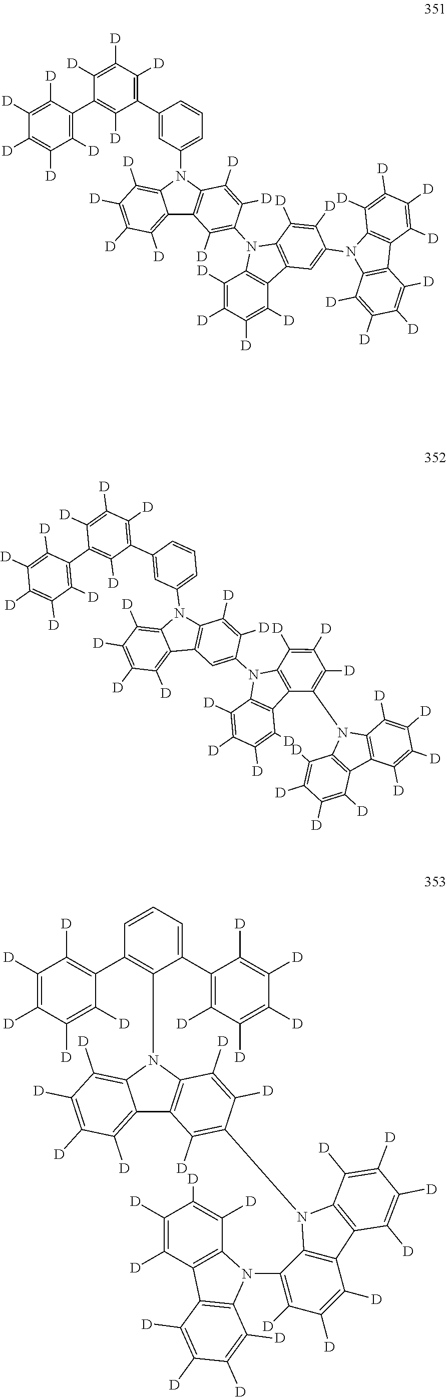

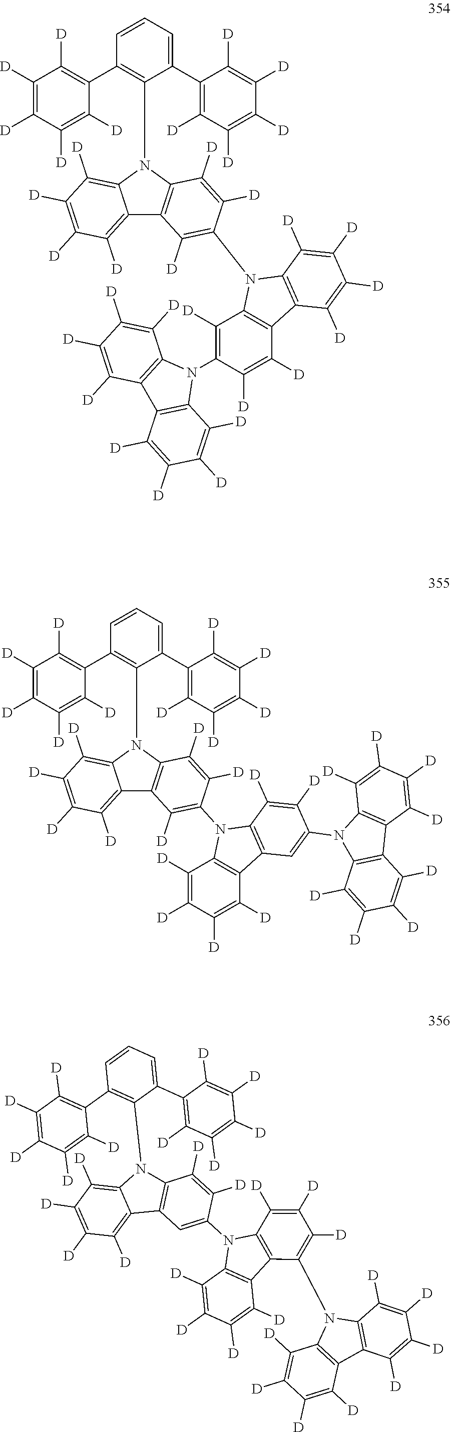

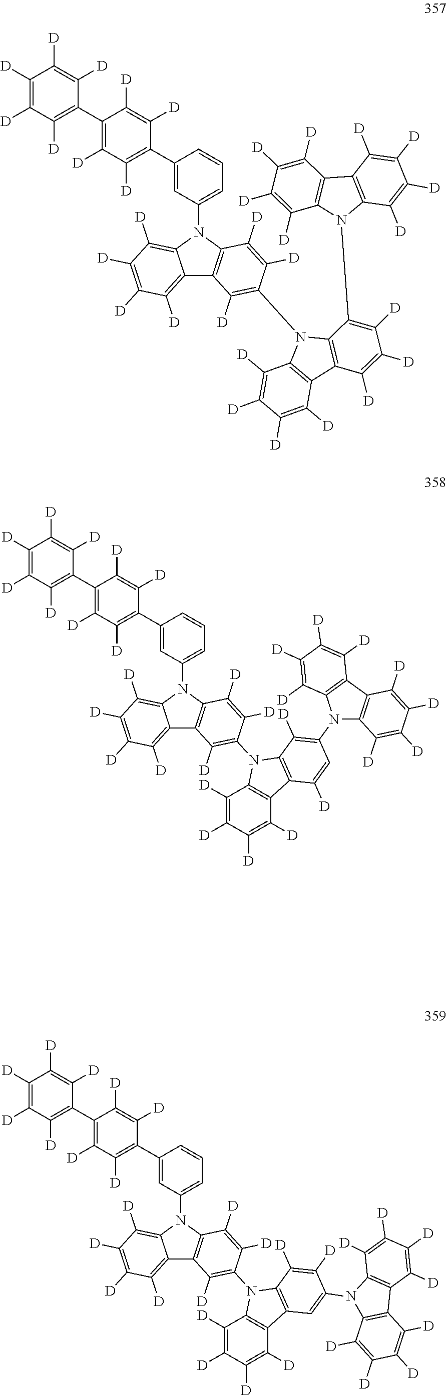

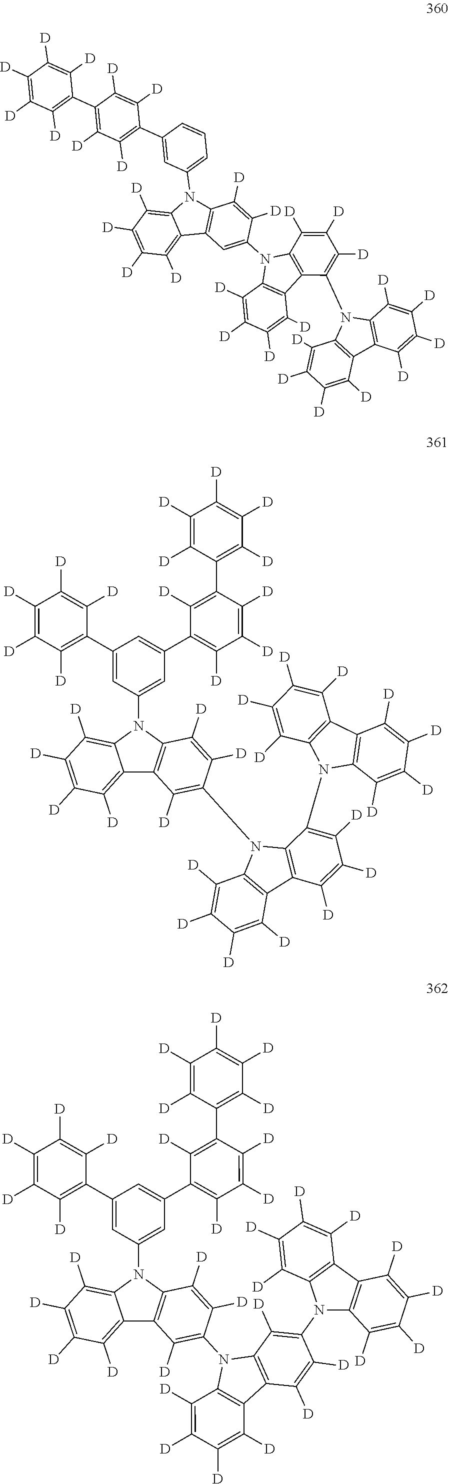

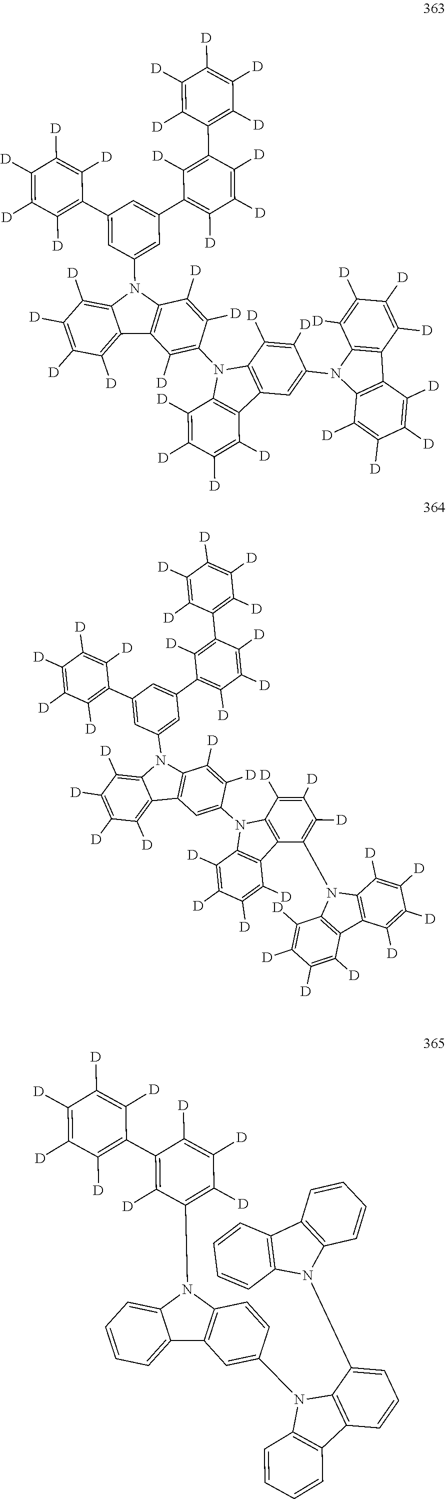

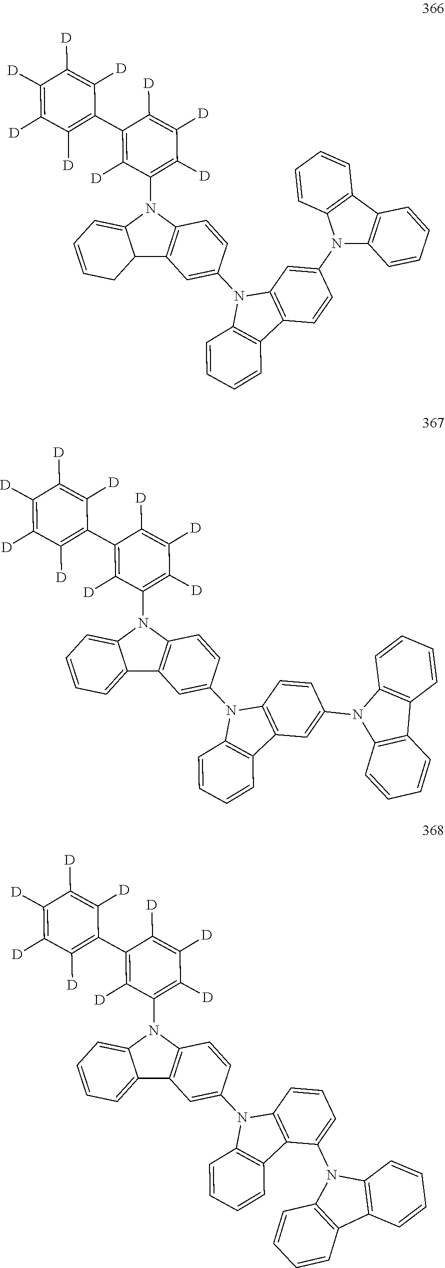 [SMILES: [2H]c1c([2H])c([2H])c(-c2c([2H])c([2H])c([2H])c(N3C4=CC=CCC4c4cc(-n5c6ccccc6c6ccc(-n7c8ccccc8c8ccccc87)cc65)ccc43)c2[2H])c([2H])c1[2H] ; [2H]c1c([2H])c([2H])c(-c2c([2H])c([2H])c([2H])c(-n3c4ccccc4c4cc(-n5c6ccccc6c6cc(-n7c8ccccc8c8ccccc87)ccc65)ccc43)c2[2H])c([2H])c1[2H] ; [2H]c1c([2H])c([2H])c(-c2c([2H])c([2H])c([2H])c(-n3c4ccccc4c4cc(-n5c6ccccc6c6c(-n7c8ccccc8c8ccccc87)cccc65)ccc43)c2[2H])c([2H])c1[2H]]

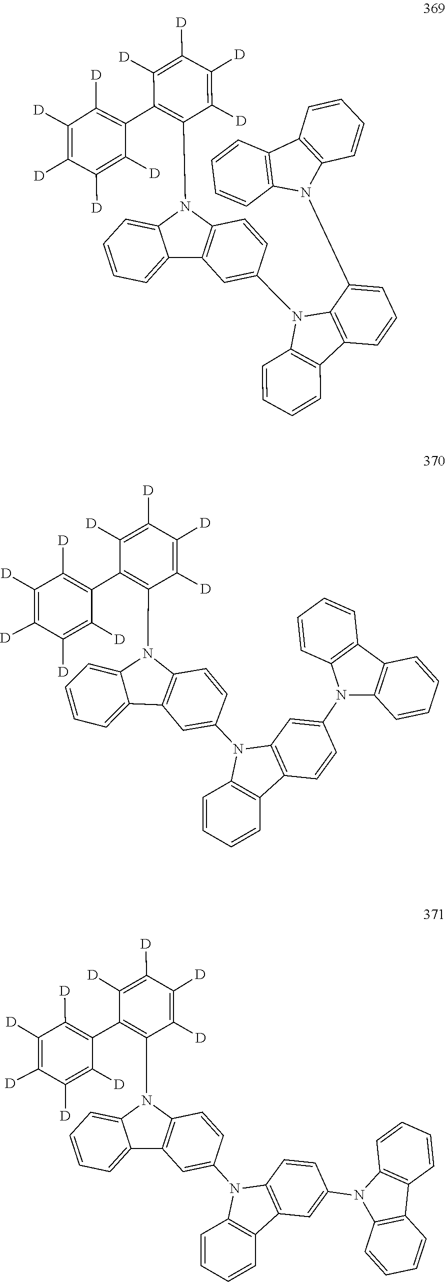 [SMILES: [2H]c1c([2H])c([2H])c(-c2c([2H])c([2H])c([2H])c([2H])c2-n2c3ccccc3c3cc(-n4c5ccccc5c5cccc(-n6c7ccccc7c7ccccc76)c54)ccc32)c([2H])c1[2H] ; [2H]c1c([2H])c([2H])c(-c2c([2H])c([2H])c([2H])c([2H])c2-n2c3ccccc3c3cc(-n4c5ccccc5c5ccc(-n6c7ccccc7c7ccccc76)cc54)ccc32)c([2H])c1[2H] ; [2H]c1c([2H])c([2H])c(-c2c([2H])c([2H])c([2H])c([2H])c2Cn2c3ccccc3c3cc(-n4c5ccccc5c5cc(-n6c7ccccc7c7ccccc76)ccc54)ccc32)c([2H])c1[2H]]

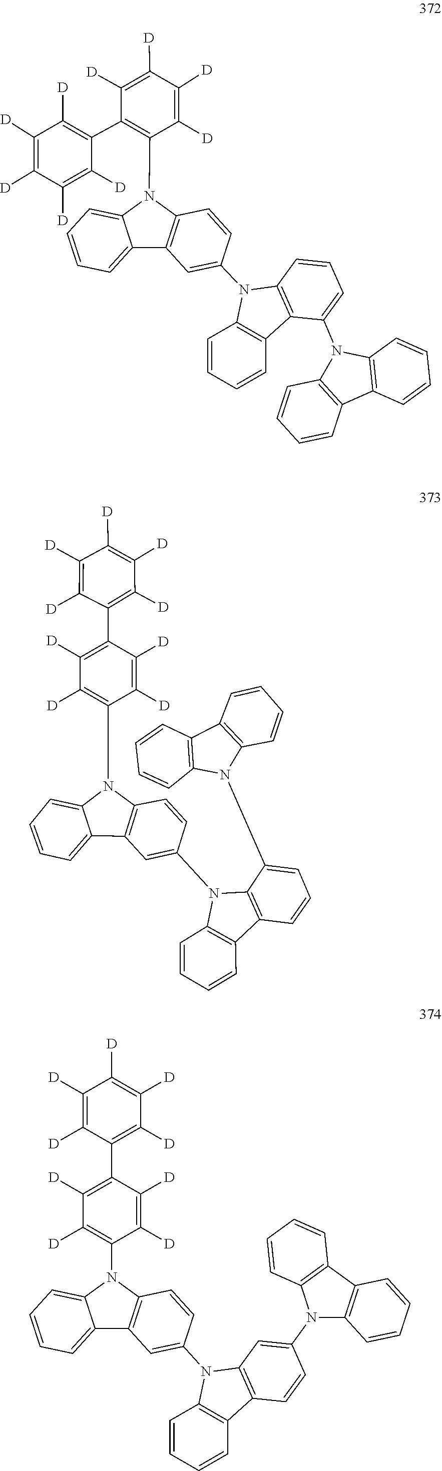 [SMILES: [2H]c1c([2H])c([2H])c(-c2c([2H])c([2H])c([2H])c([2H])c2-n2c3ccccc3c3cc(-n4c5ccccc5c5c(-n6c7ccccc7c7ccccc76)cccc54)ccc32)c([2H])c1[2H] ; [2H]c1c([2H])c([2H])c(-c2c([2H])c([2H])c(-n3c4ccccc4c4cc(-n5c6ccccc6c6cccc(-n7c8ccccc8c8ccccc87)c65)ccc43)c([2H])c2[2H])c([2H])c1[2H] ; [2H]c1c([2H])c([2H])c(-c2c([2H])c([2H])c(-n3c4ccccc4c4cc(-n5c6ccccc6c6ccc(-n7c8ccccc8c8ccccc87)cc65)ccc43)c([2H])c2[2H])c([2H])c1[2H]]

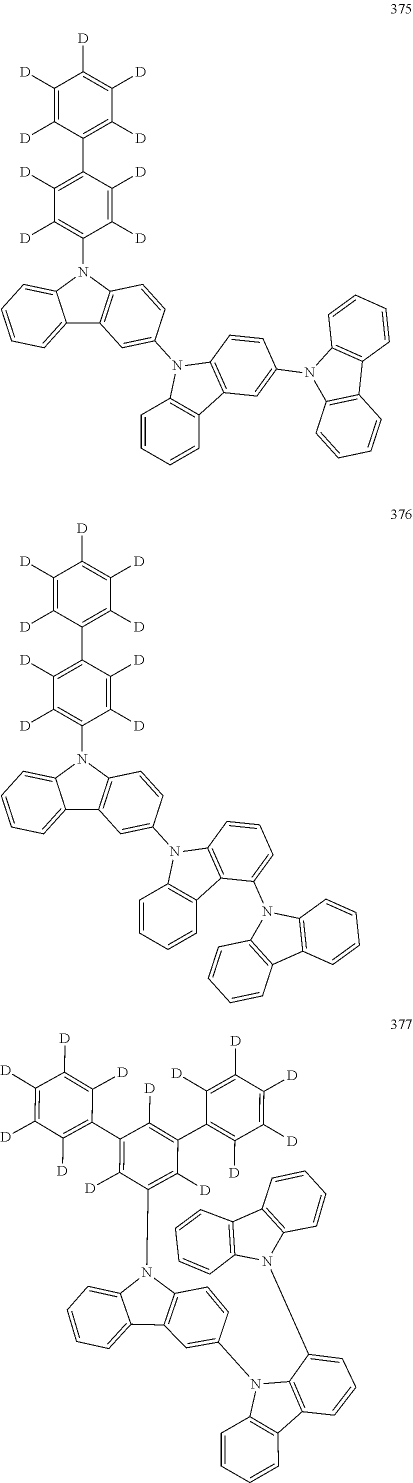

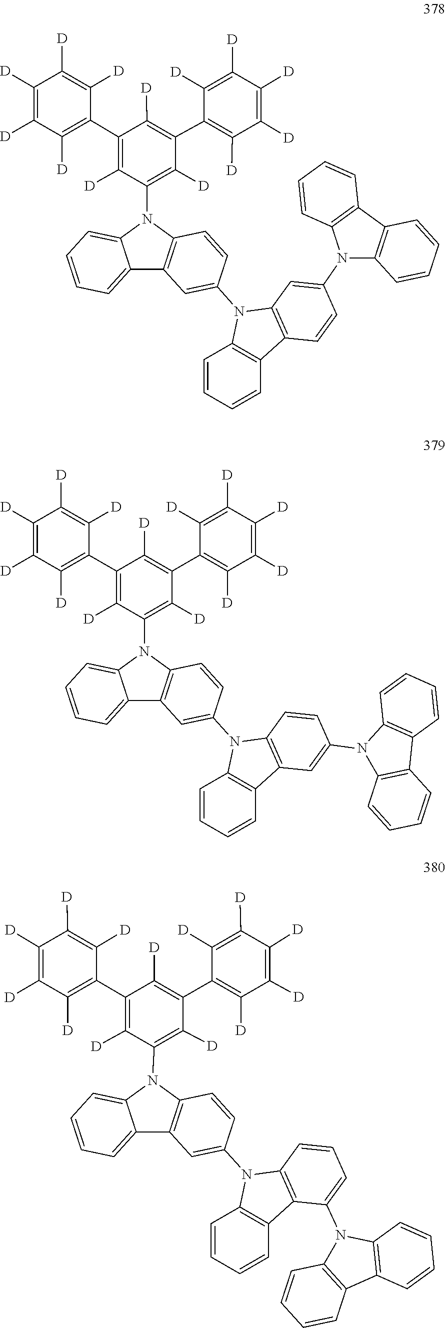

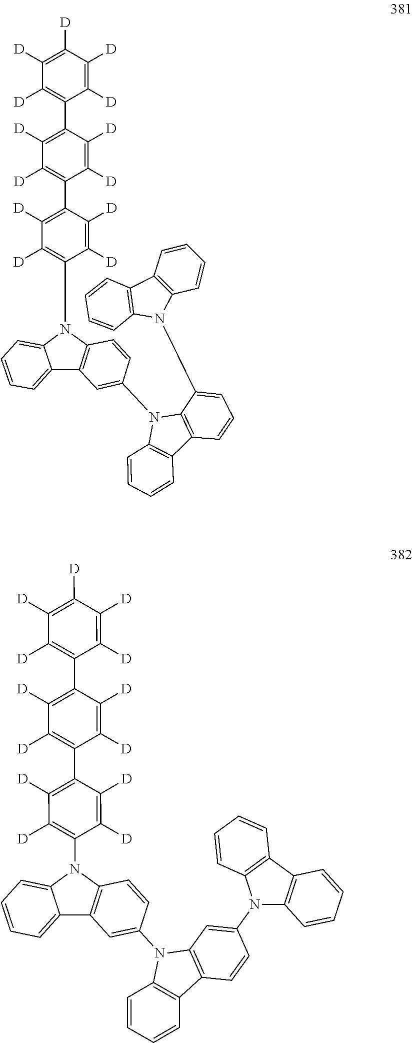 [SMILES: [2H]c1c([2H])c([2H])c(-c2c([2H])c([2H])c(-c3c([2H])c([2H])c(-n4c5ccccc5c5cc(-n6c7ccccc7c7cccc(-n8c9ccccc9c9ccccc98)c76)ccc54)c([2H])c3[2H])c([2H])c2[2H])c([2H])c1[2H] ; [2H]c1c([2H])c([2H])c(-c2c([2H])c([2H])c(-c3c([2H])c([2H])c(-n4c5ccccc5c5cc(-n6c7ccccc7c7ccc(-n8c9ccccc9c9ccccc98)cc76)ccc54)c([2H])c3[2H])c([2H])c2[2H])c([2H])c1[2H]]

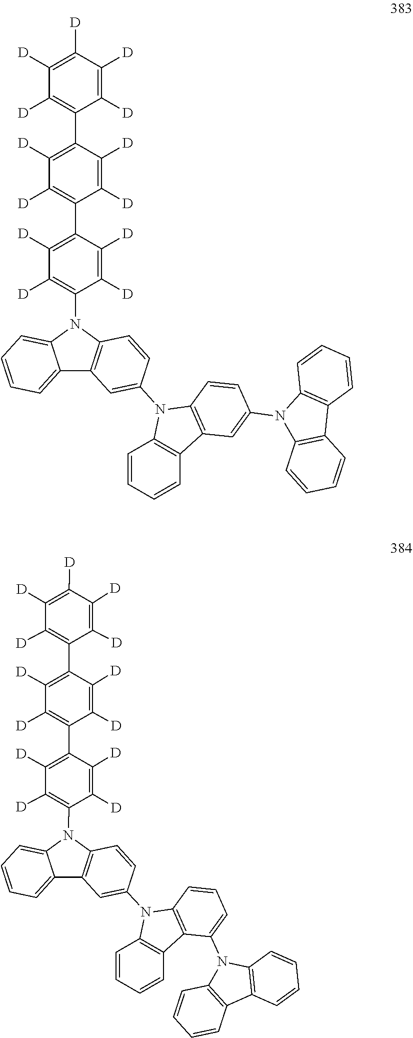 [SMILES: [2H]c1c([2H])c([2H])c(-c2c([2H])c([2H])c(-c3c([2H])c([2H])c(-n4c5ccccc5c5cc(-n6c7ccccc7c7cc(-n8c9ccccc9c9ccccc98)ccc76)ccc54)c([2H])c3[2H])c([2H])c2[2H])c([2H])c1[2H] ; [2H]c1c([2H])c([2H])c(-c2c([2H])c([2H])c(-c3c([2H])c([2H])c(-n4c5ccccc5c5cc(-n6c7ccccc7c7c(-n8c9ccccc9c9ccccc98)cccc76)ccc54)c([2H])c3[2H])c([2H])c2[2H])c([2H])c1[2H]]

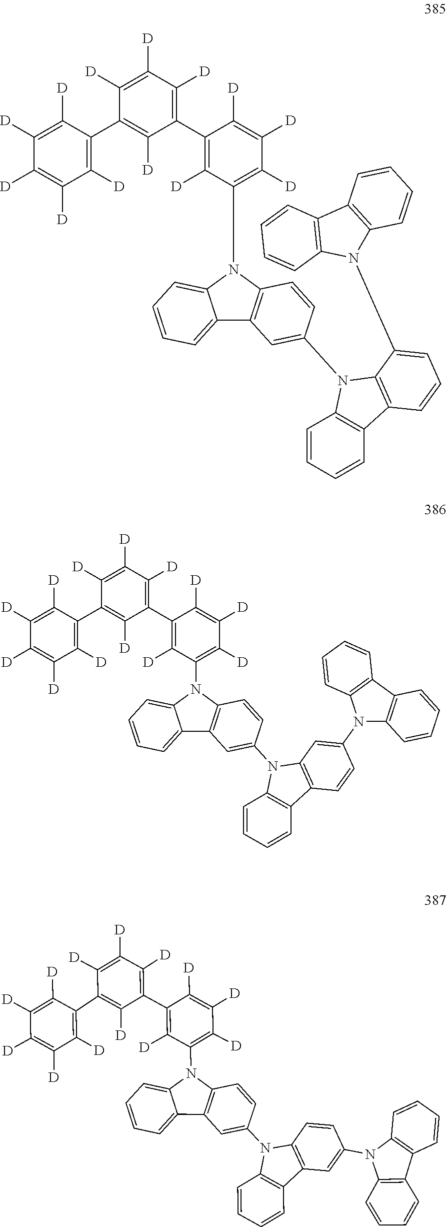

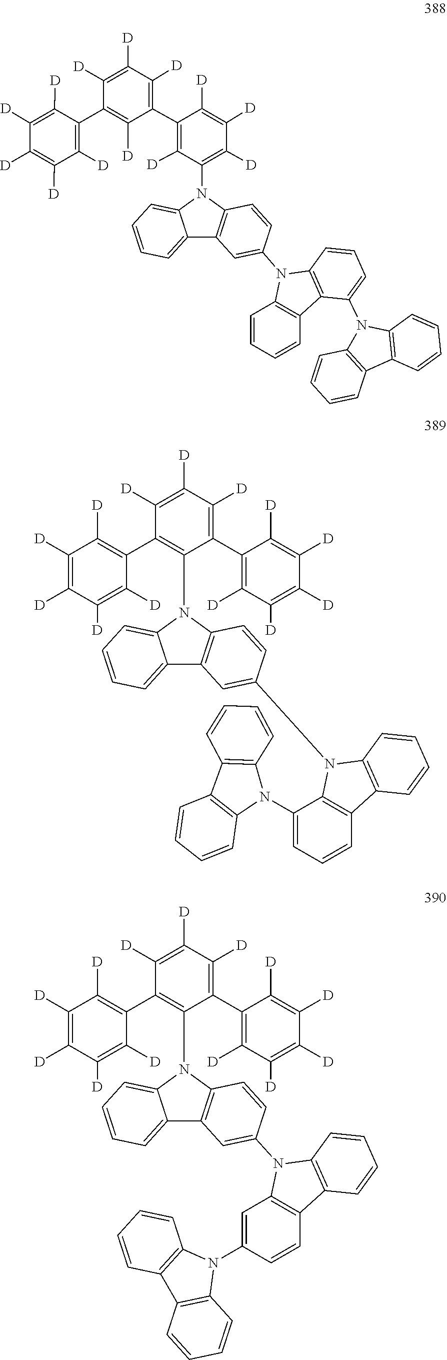

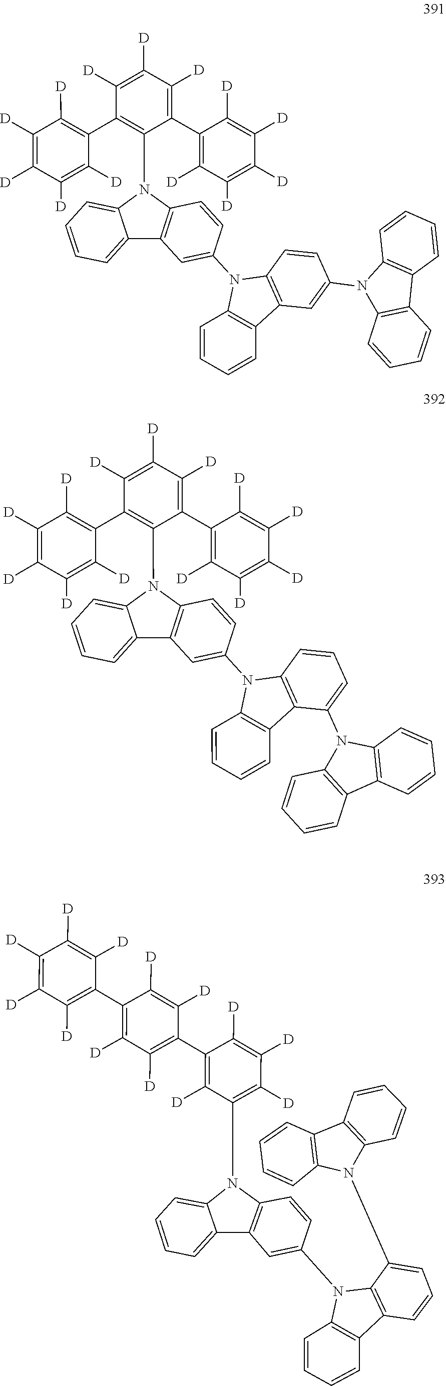

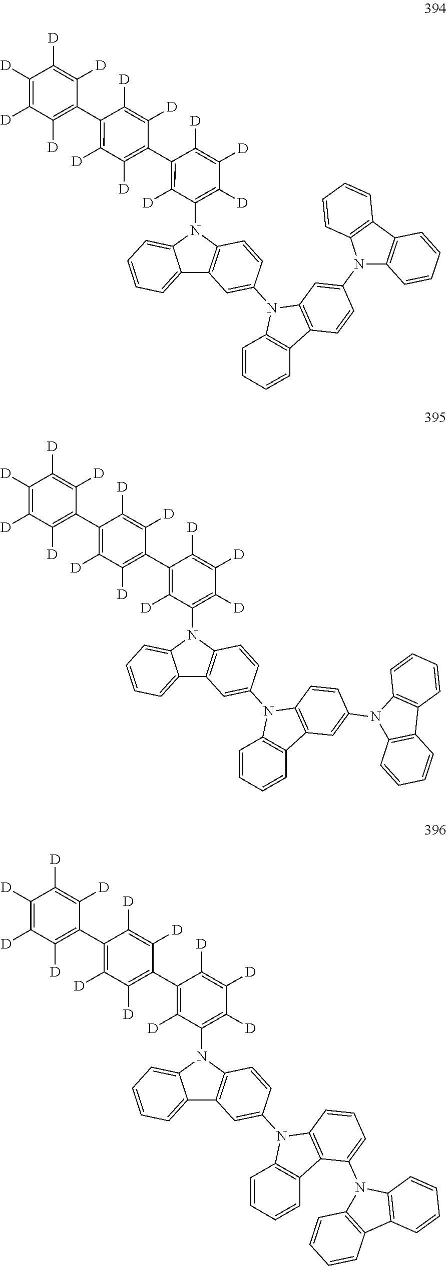

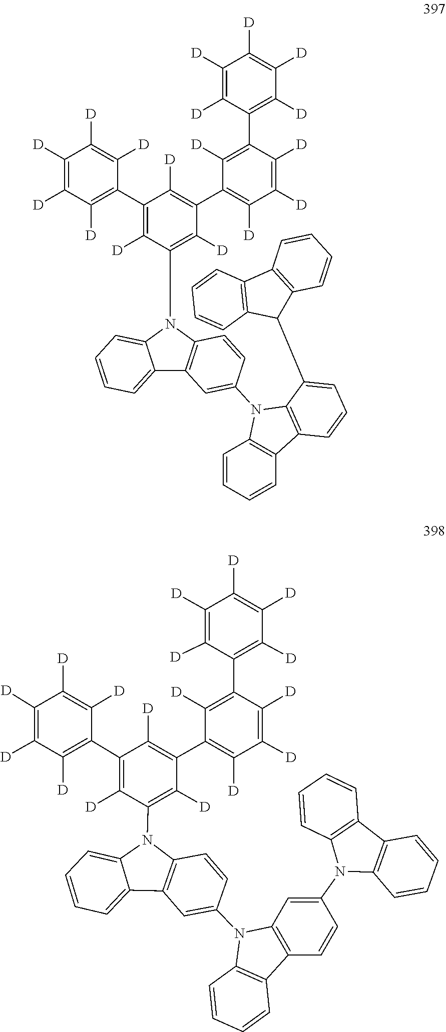 [SMILES: [2H]c1c([2H])c([2H])c(-c2c([2H])c([2H])c([2H])c(-c3c([2H])c(-c4c([2H])c([2H])c([2H])c([2H])c4[2H])c([2H])c(-n4c5ccccc5c5cc(-n6c7ccccc7c7cccc(C8c9ccccc9-c9ccccc98)c76)ccc54)c3[2H])c2[2H])c([2H])c1[2H] ; [2H]c1c([2H])c([2H])c(-c2c([2H])c([2H])c([2H])c(-c3c([2H])c(-c4c([2H])c([2H])c([2H])c([2H])c4[2H])c([2H])c(-n4c5ccccc5c5cc(-n6c7ccccc7c7ccc(-n8c9ccccc9c9ccccc98)cc76)ccc54)c3[2H])c2[2H])c([2H])c1[2H]]

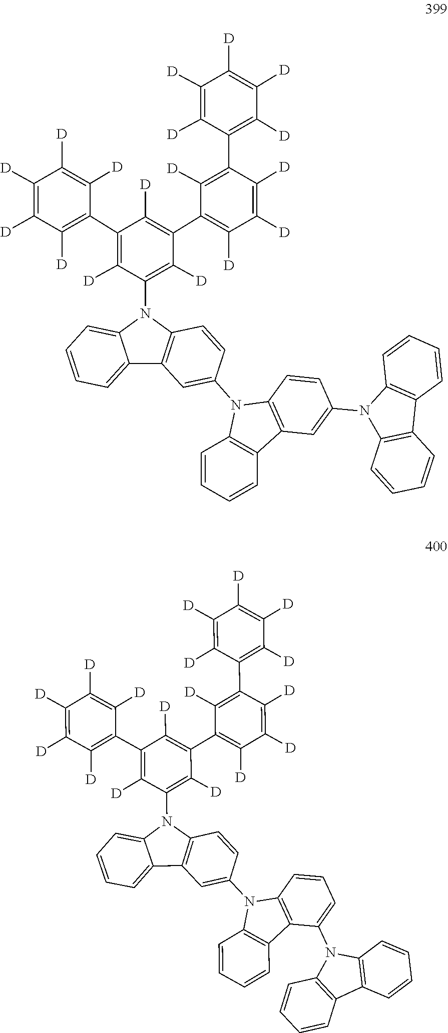 [SMILES: [2H]c1c([2H])c([2H])c(-c2c([2H])c([2H])c([2H])c(-c3c([2H])c(-c4c([2H])c([2H])c([2H])c([2H])c4[2H])c([2H])c(-n4c5ccccc5c5cc(-n6c7ccccc7c7cc(-n8c9ccccc9c9ccccc98)ccc76)ccc54)c3[2H])c2[2H])c([2H])c1[2H] ; [2H]c1c([2H])c([2H])c(-c2c([2H])c([2H])c([2H])c(-c3c([2H])c(-c4c([2H])c([2H])c([2H])c([2H])c4[2H])c([2H])c(-n4c5ccccc5c5cc(-n6c7ccccc7c7c(-n8c9ccccc9c9ccccc98)cccc76)ccc54)c3[2H])c2[2H])c([2H])c1[2H]]

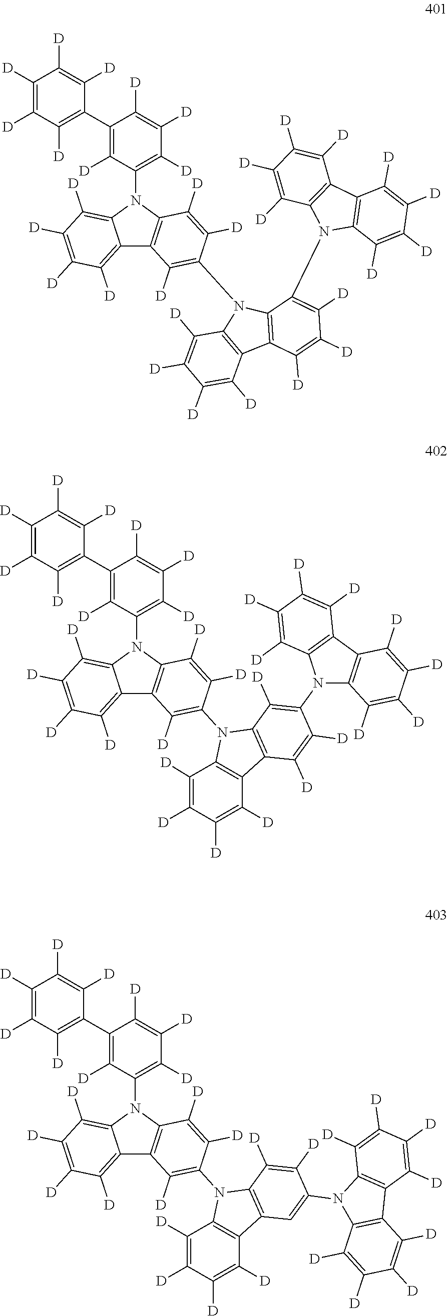

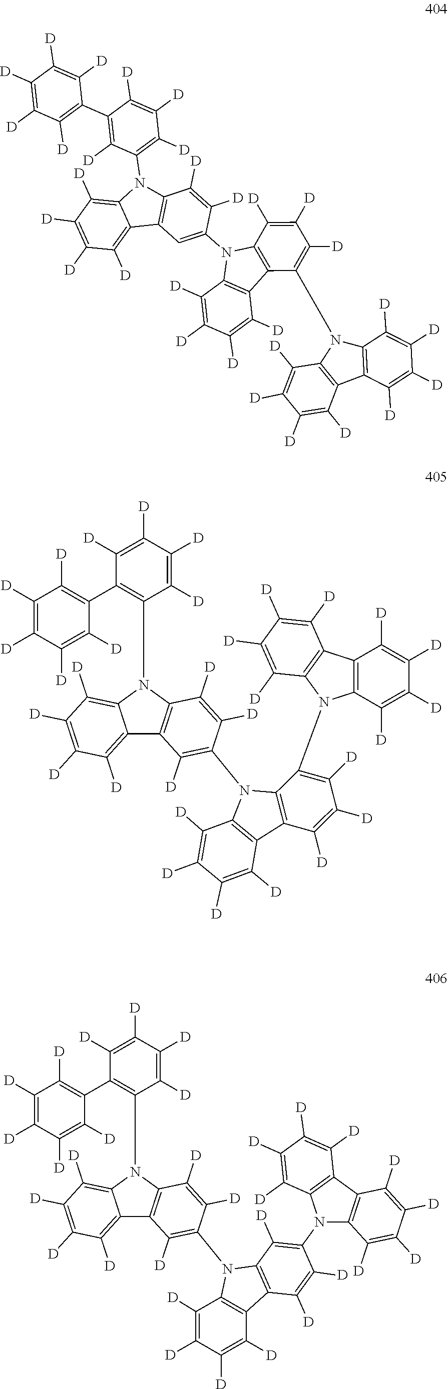

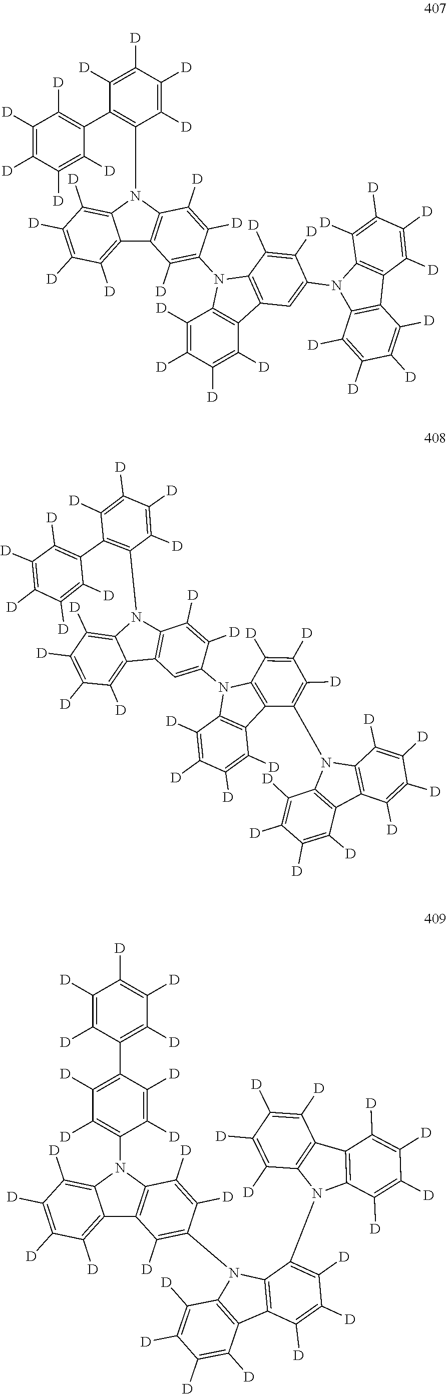

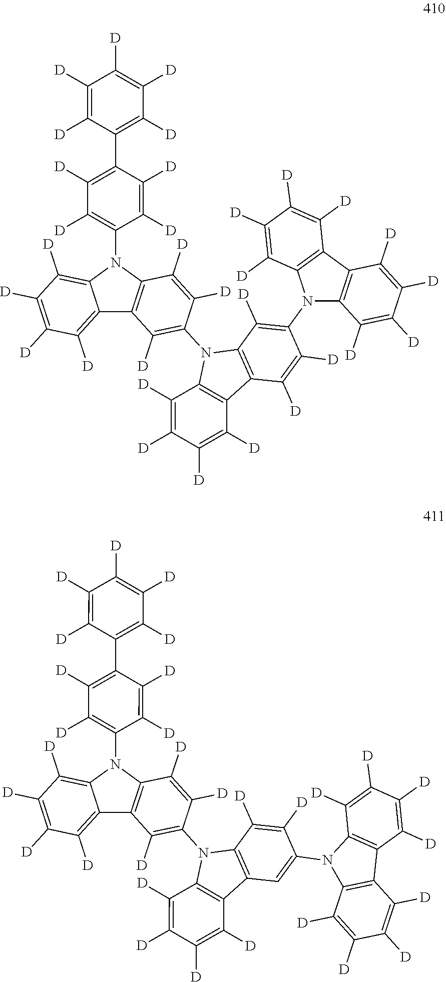

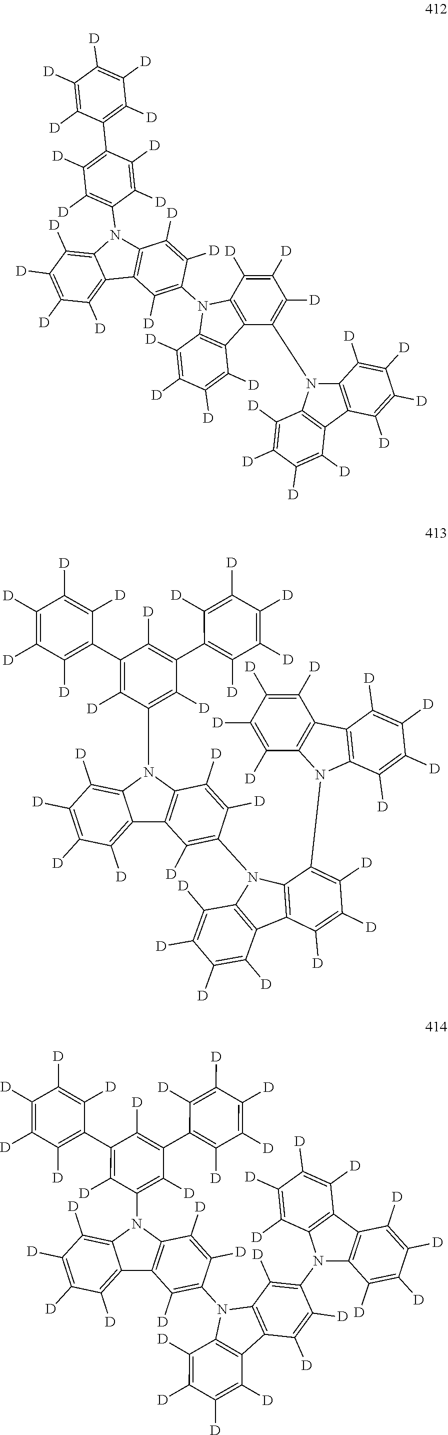

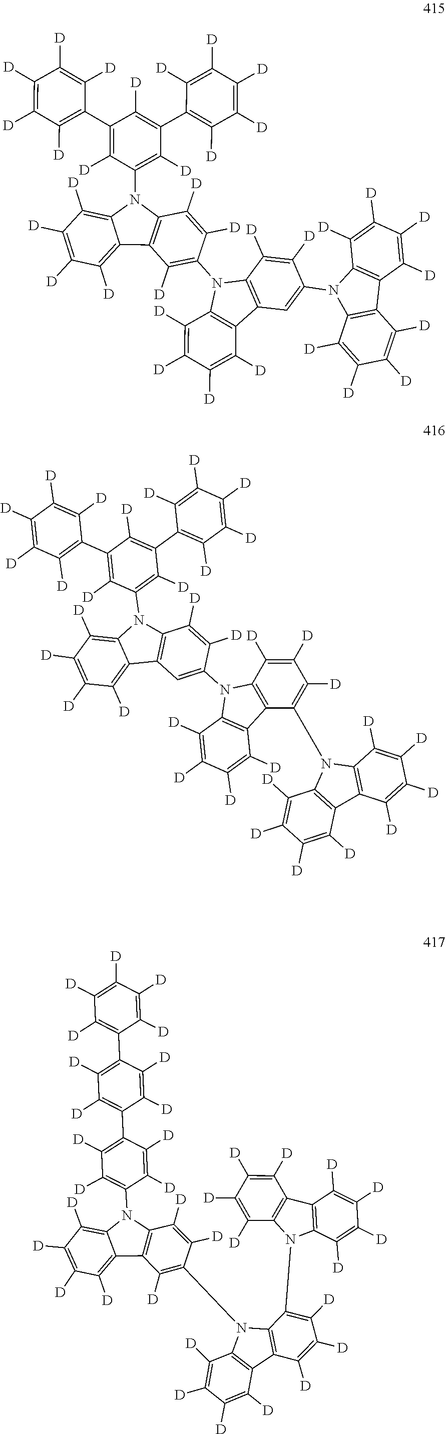

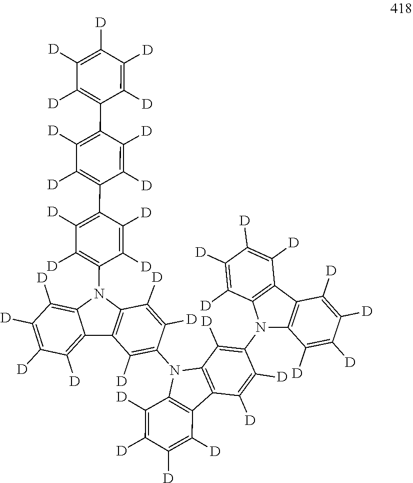 [SMILES: [2H]c1c([2H])c([2H])c(-c2c([2H])c([2H])c(-c3c([2H])c([2H])c(-n4c5c([2H])c([2H])c([2H])c([2H])c5c5c([2H])c(-n6c7c([2H])c([2H])c([2H])c([2H])c7c7c([2H])c([2H])c(-n8c9c([2H])c([2H])c([2H])c([2H])c9c9c([2H])c([2H])c([2H])c([2H])c98)c([2H])c76)c([2H])c([2H])c54)c([2H])c3[2H])c([2H])c2[2H])c([2H])c1[2H]]

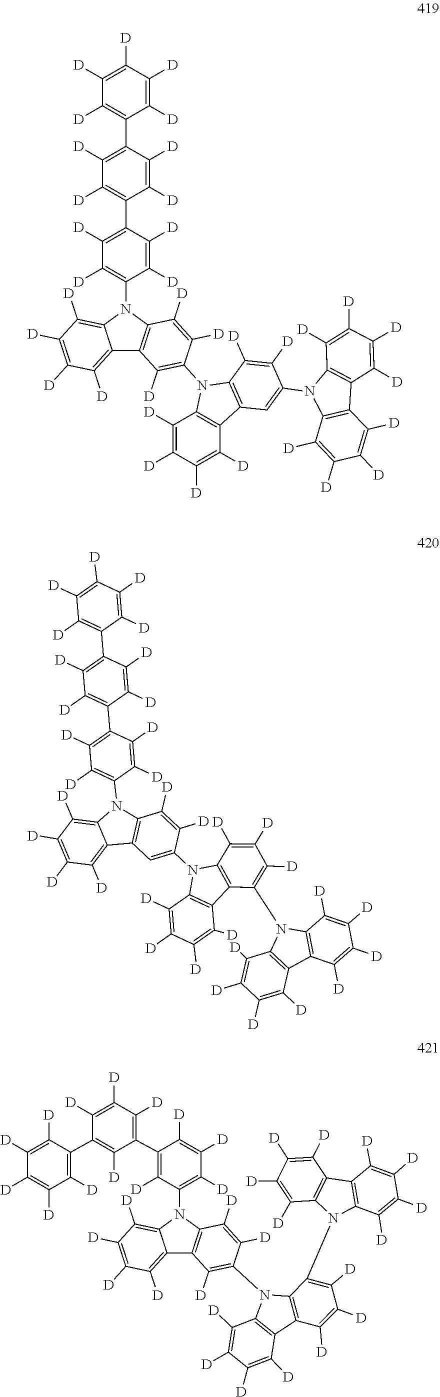

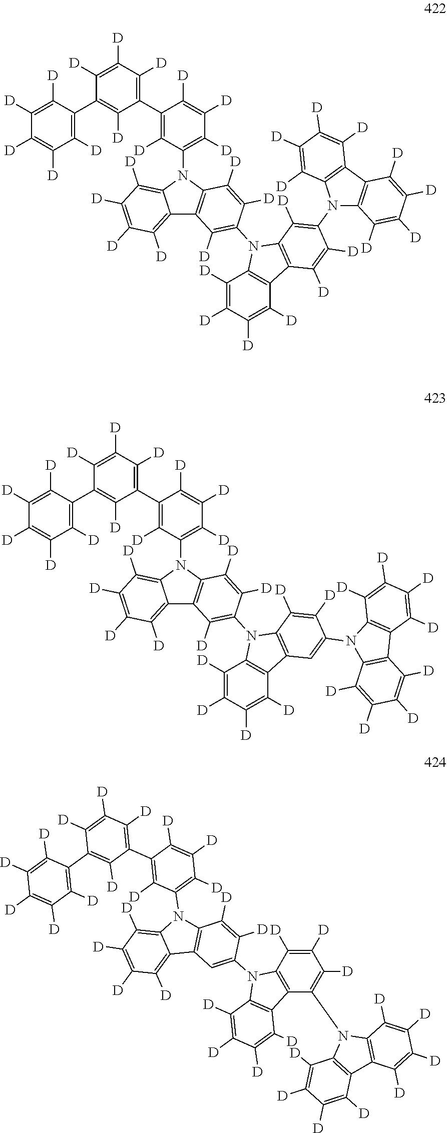

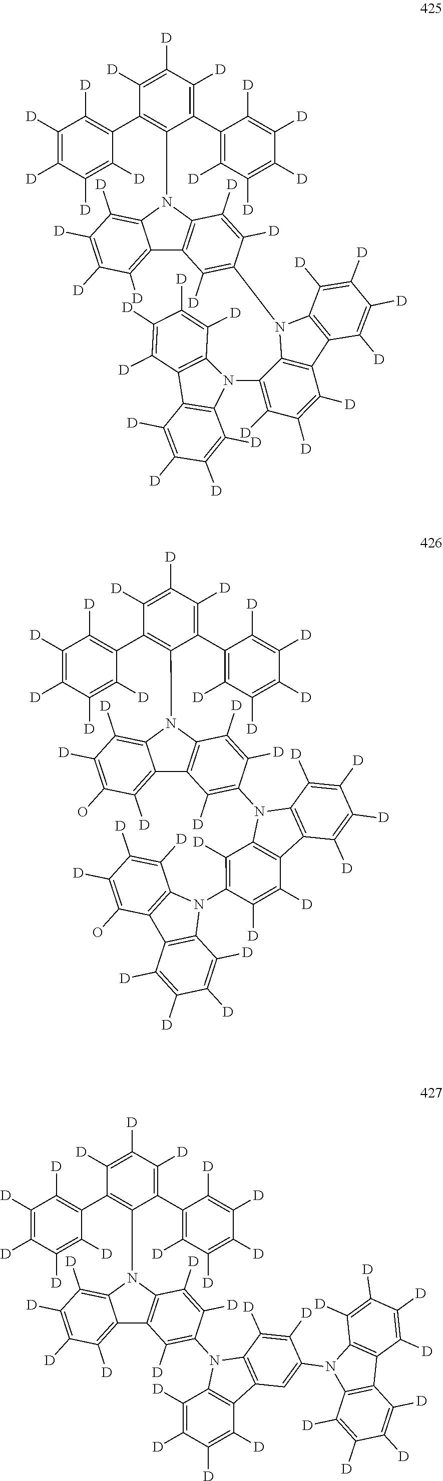

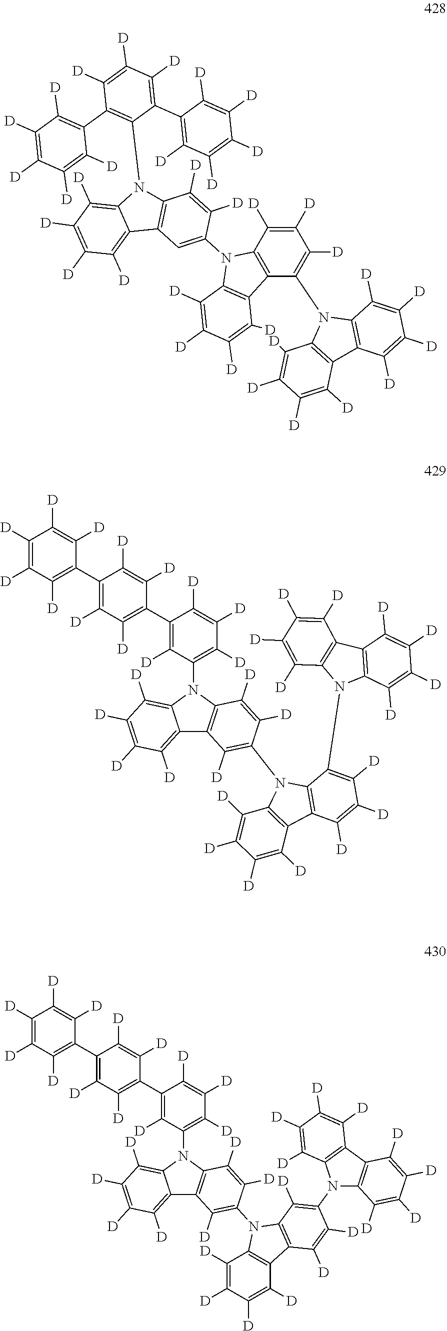

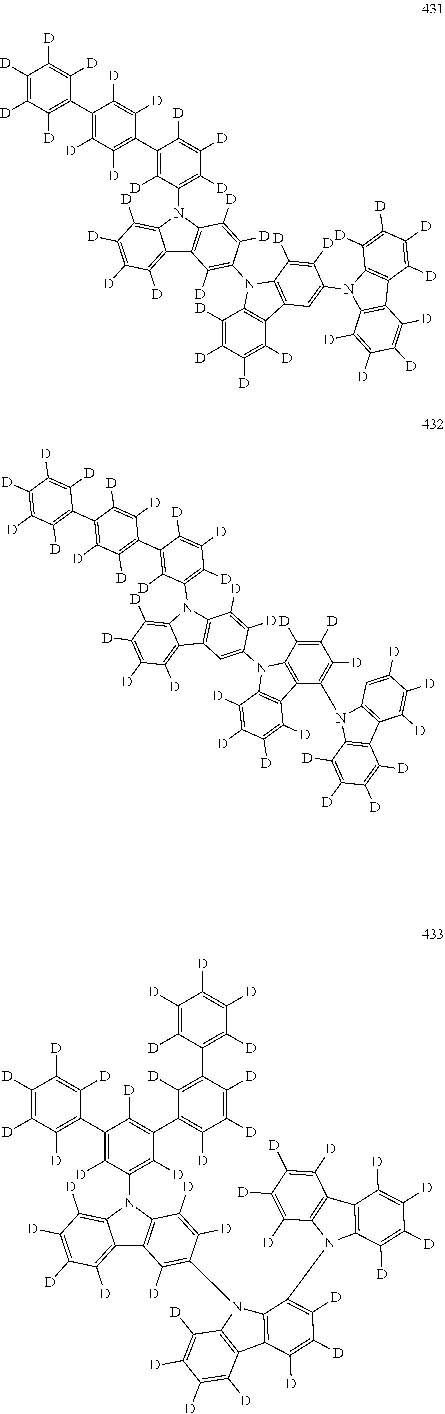

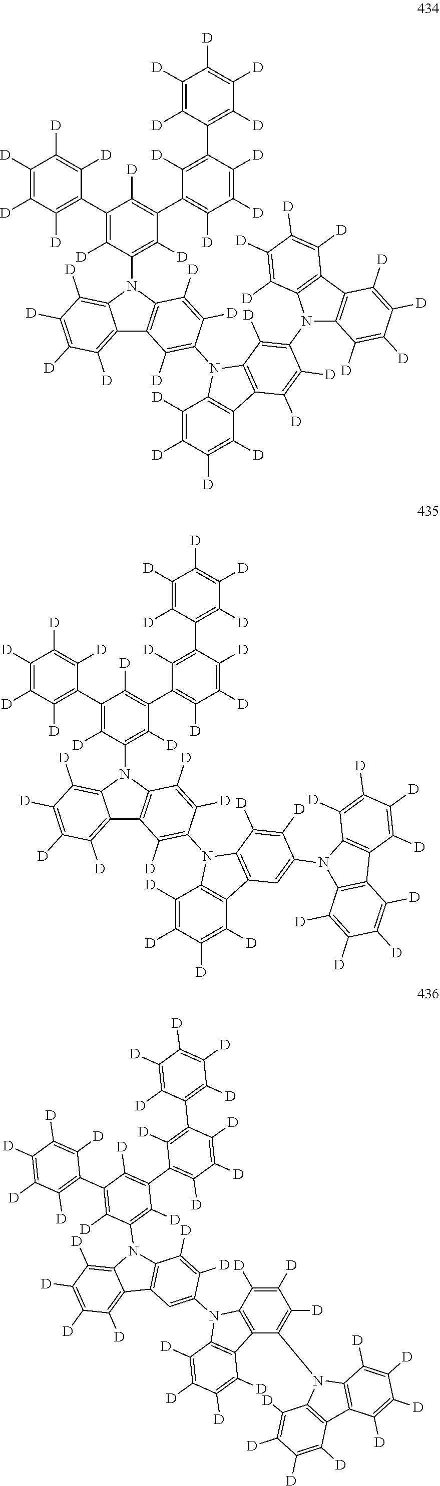

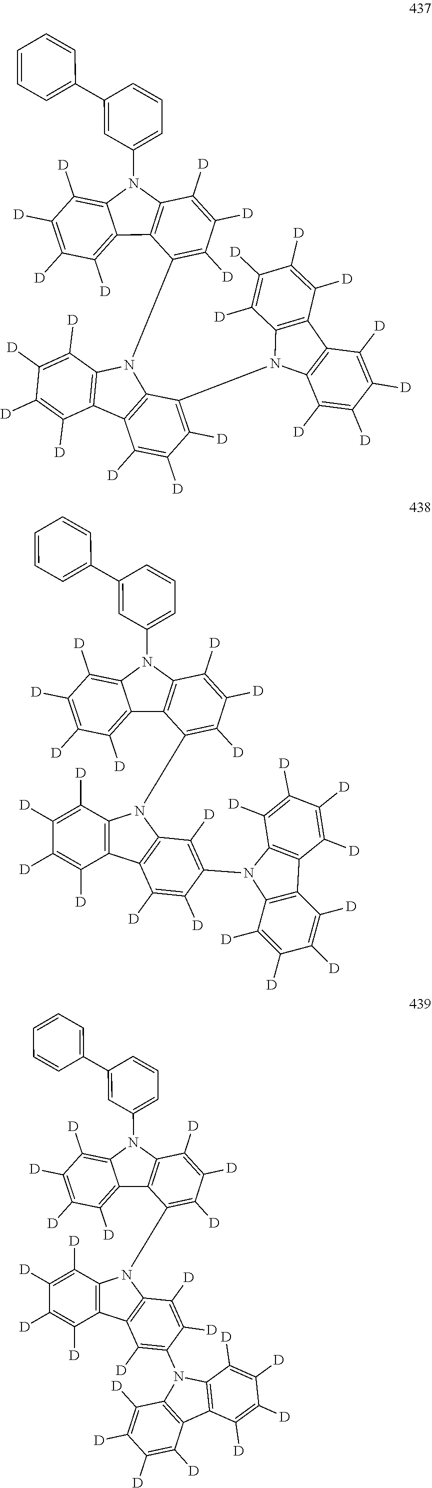

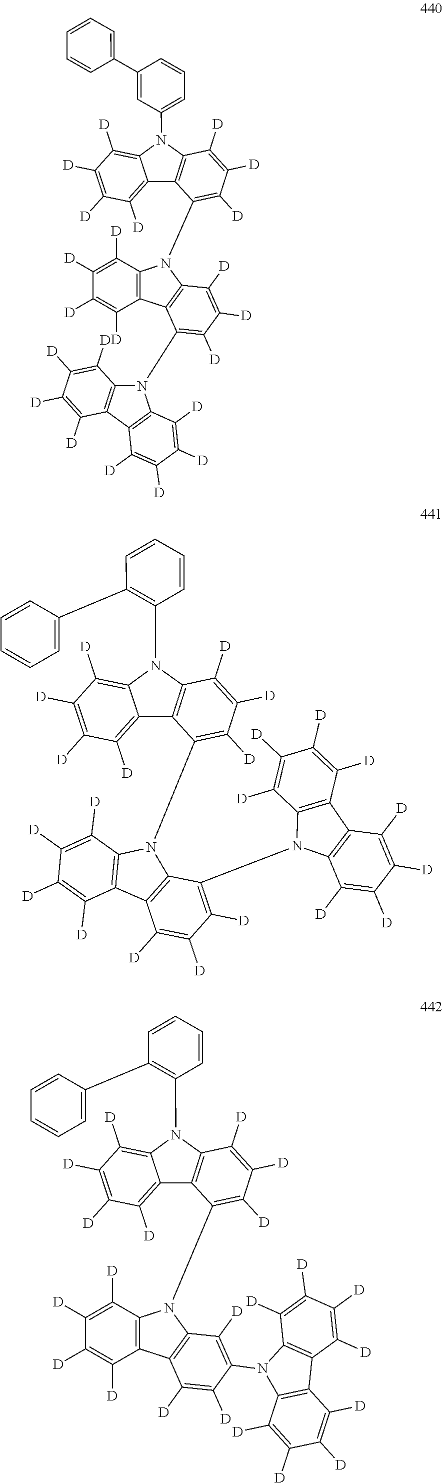

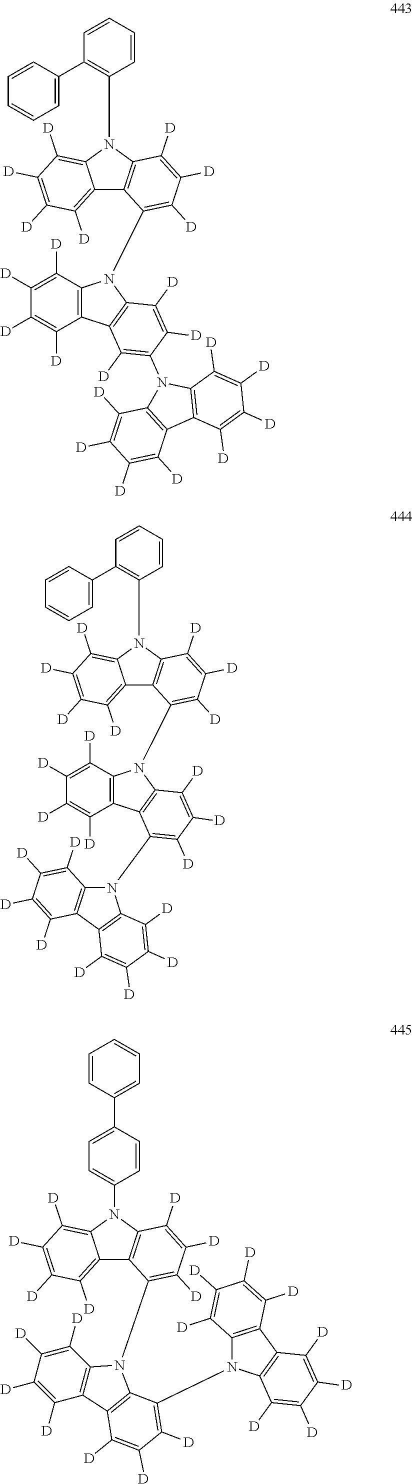

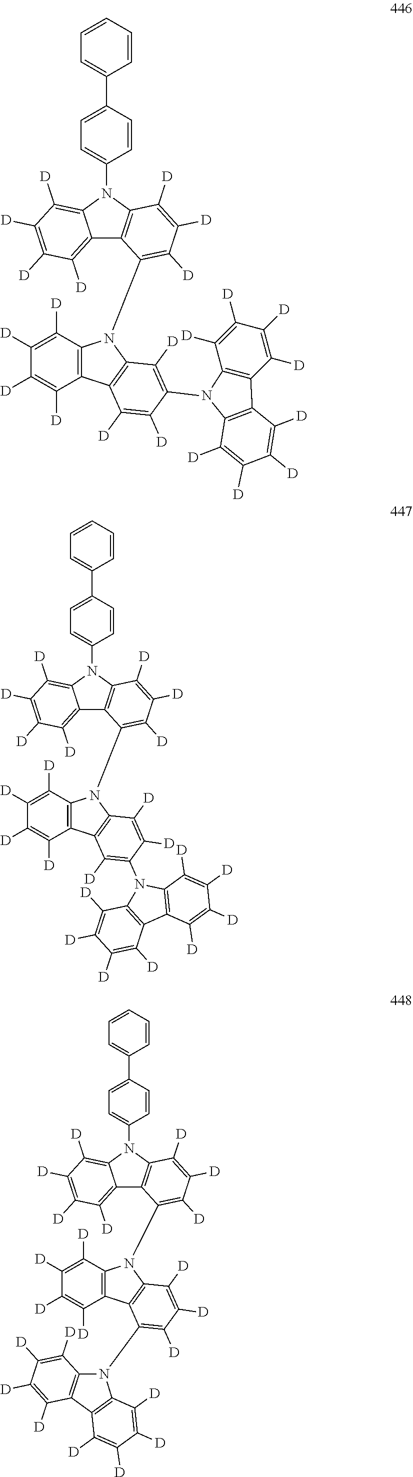

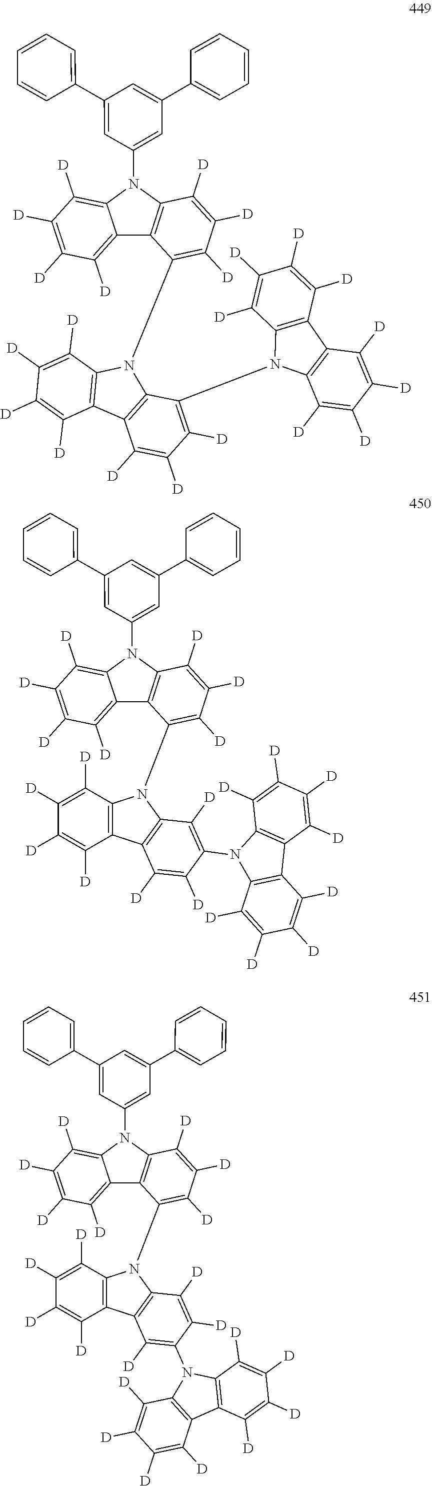

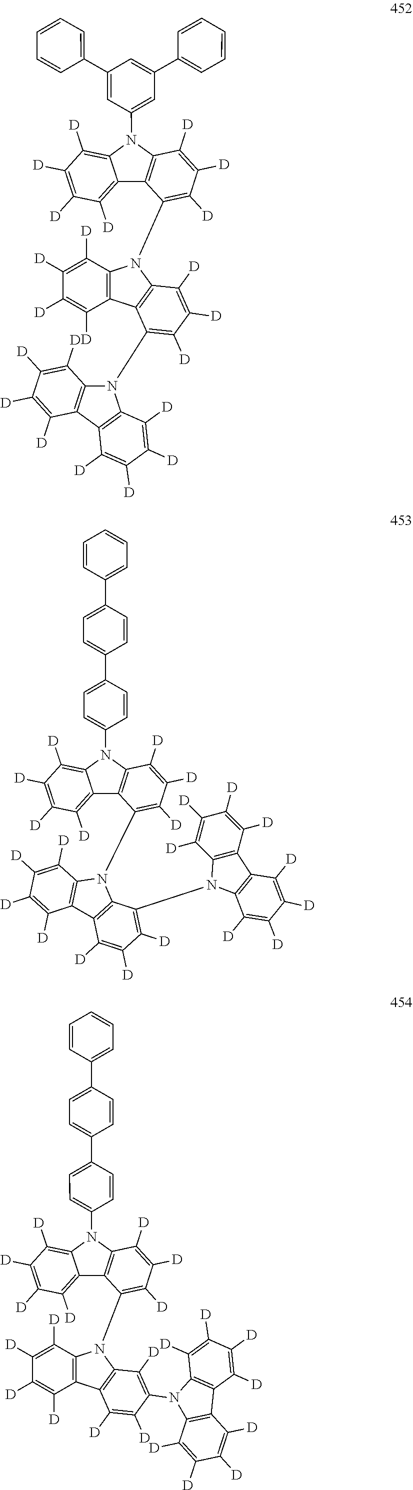

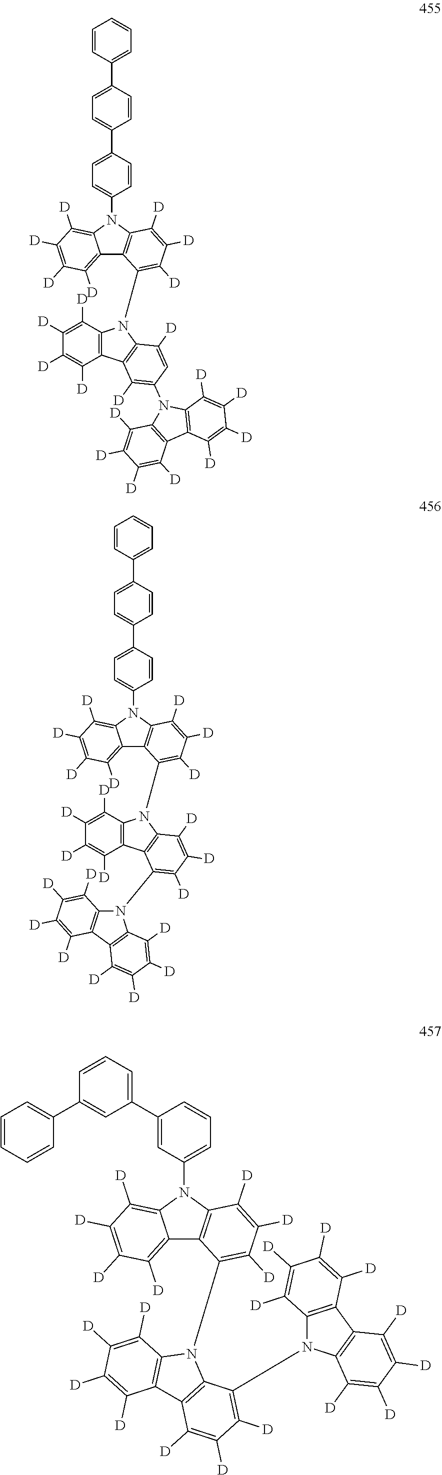

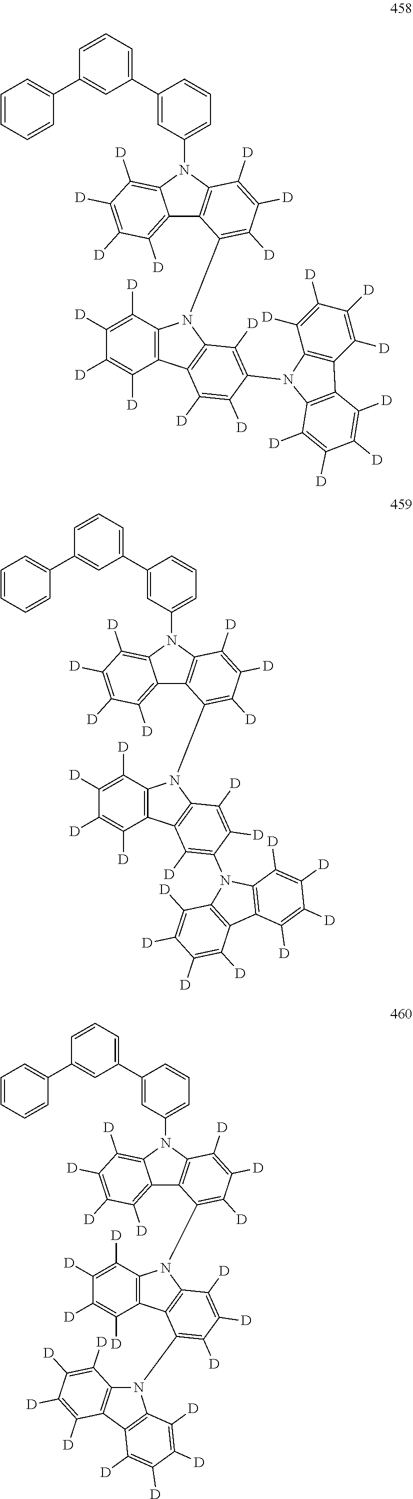

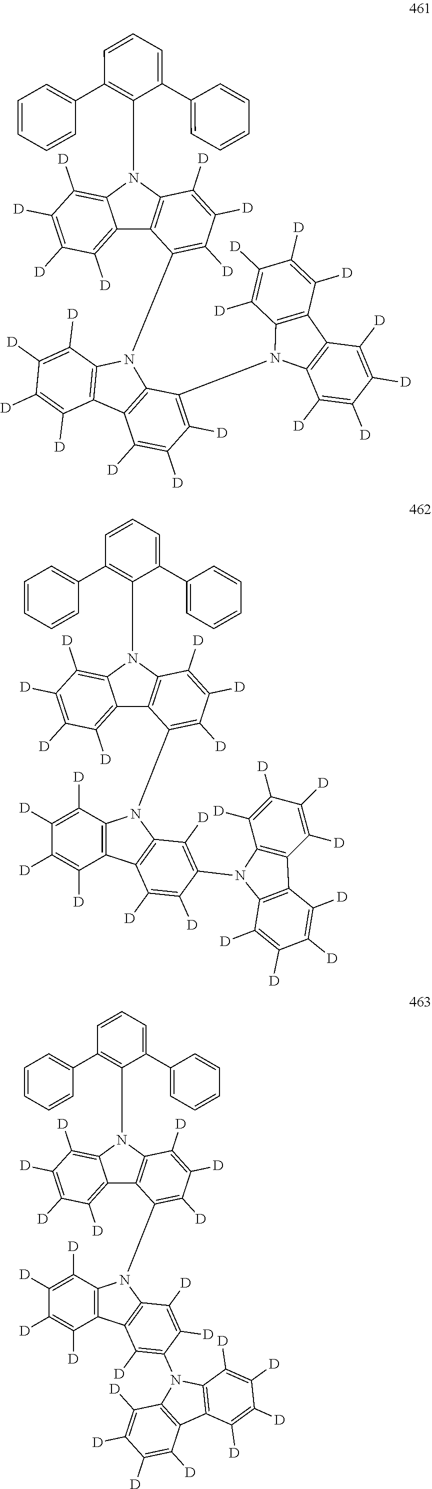

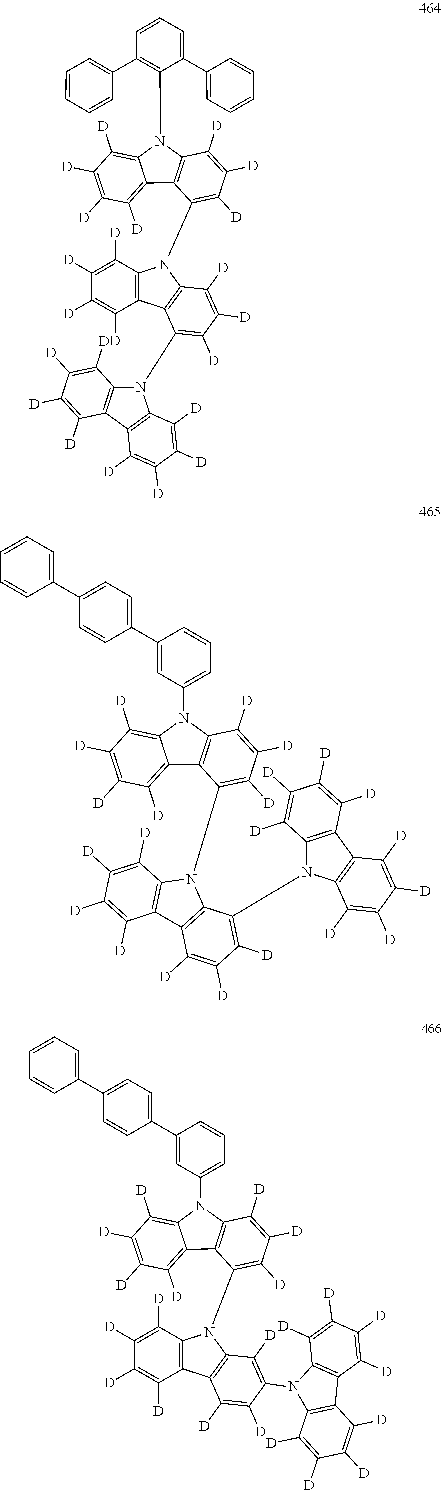

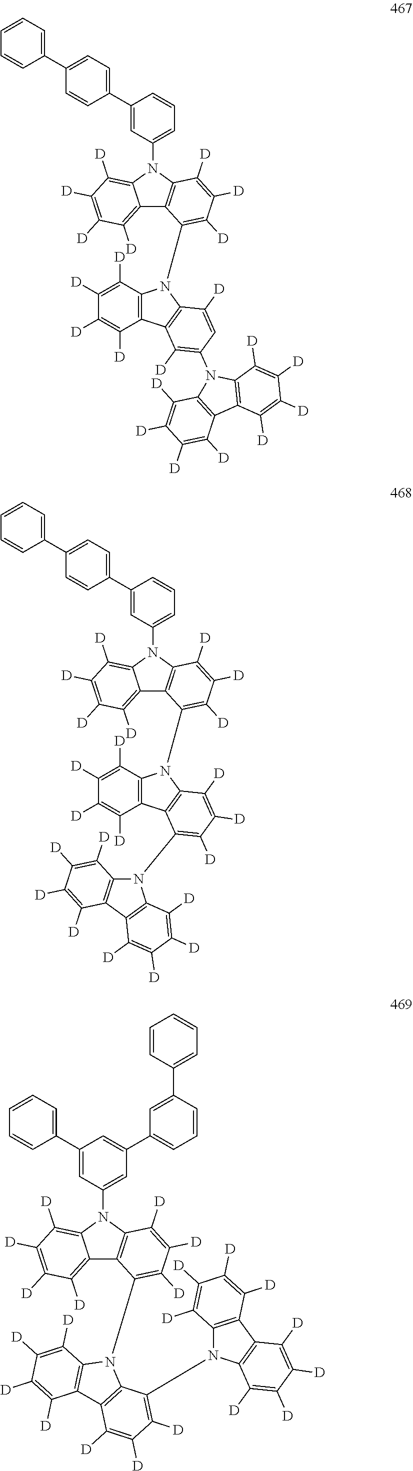

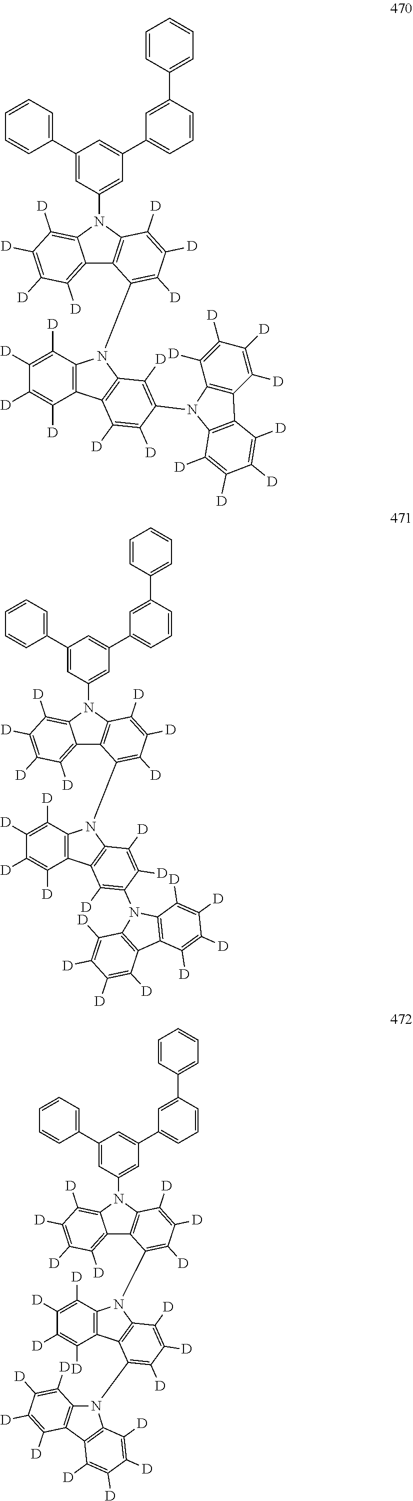

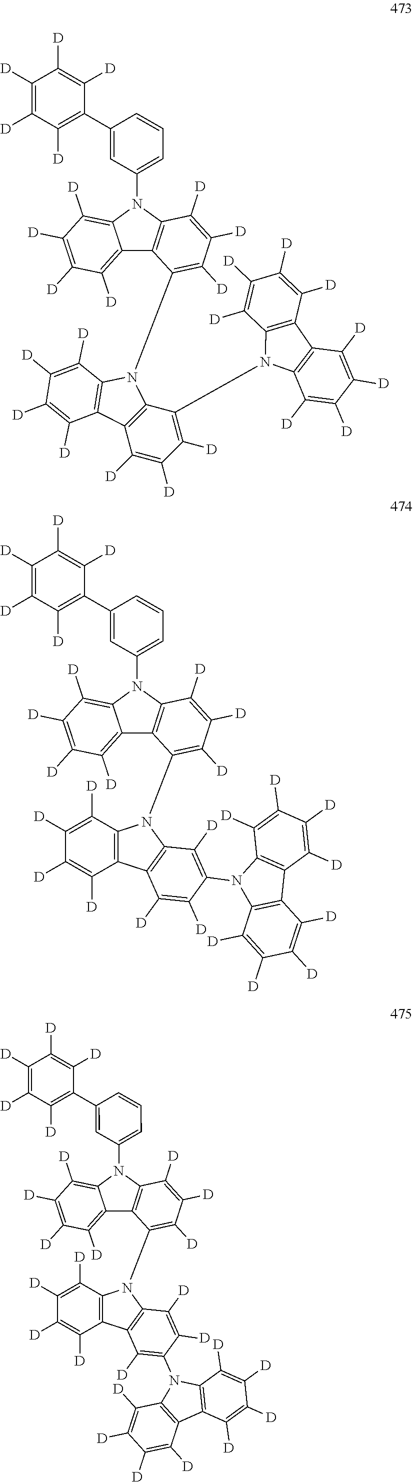

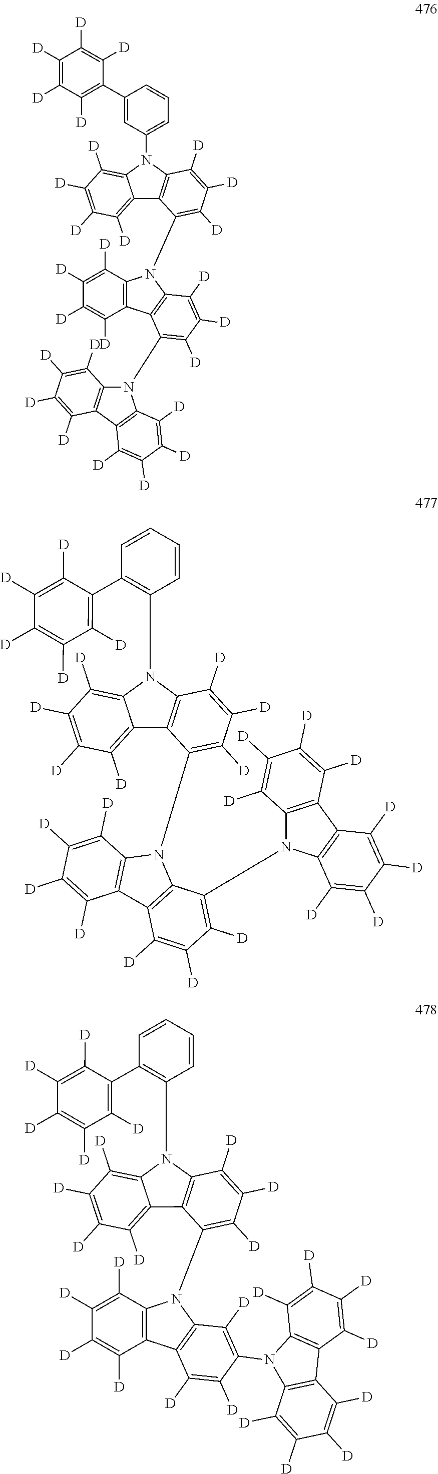

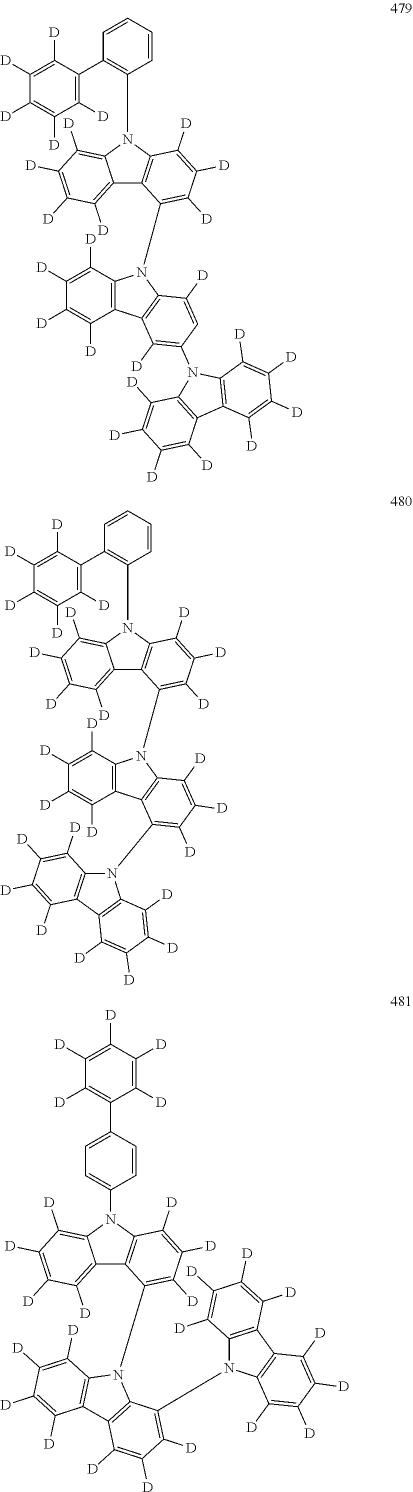

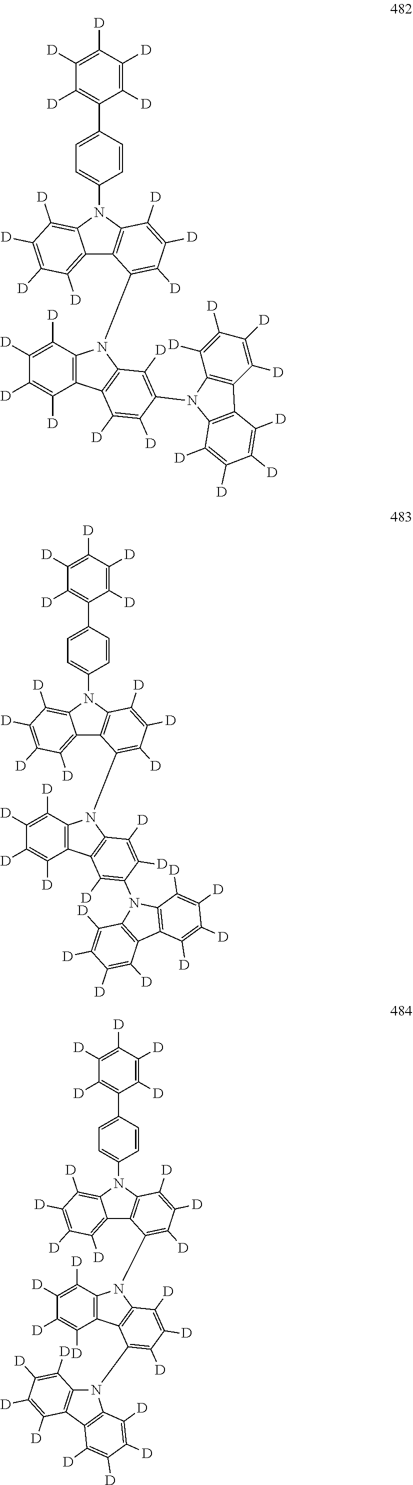

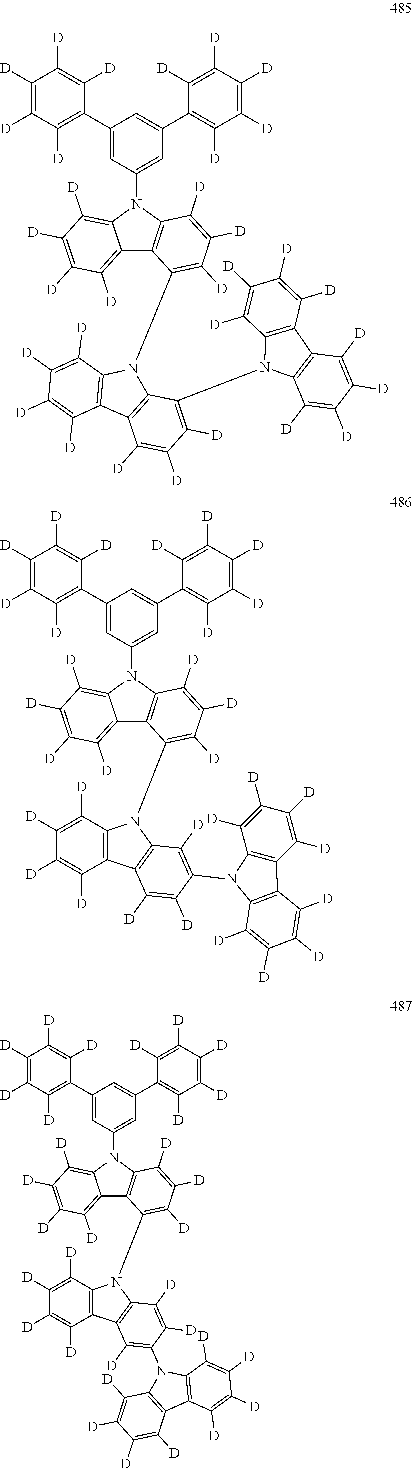

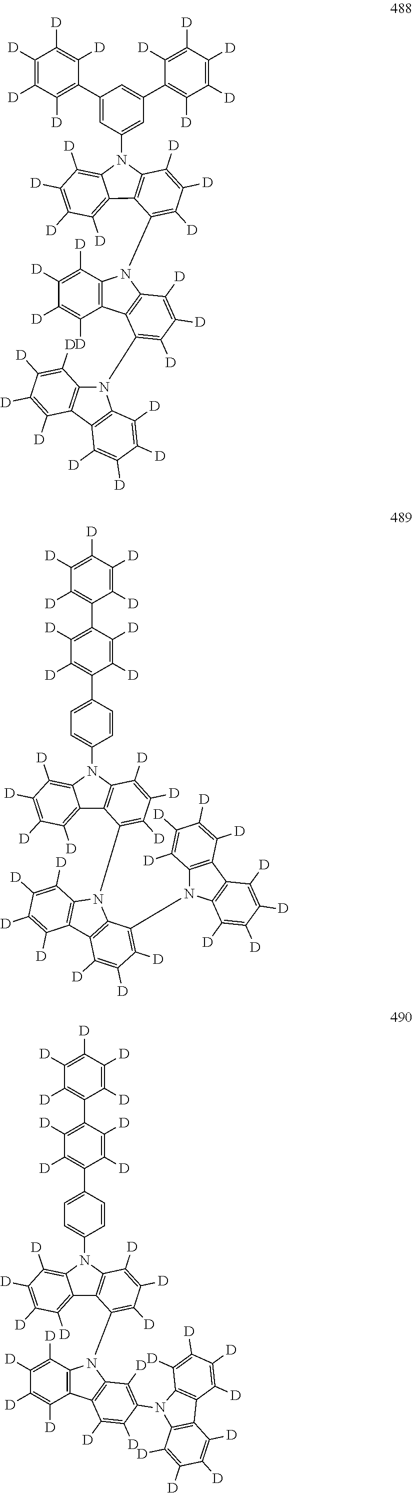

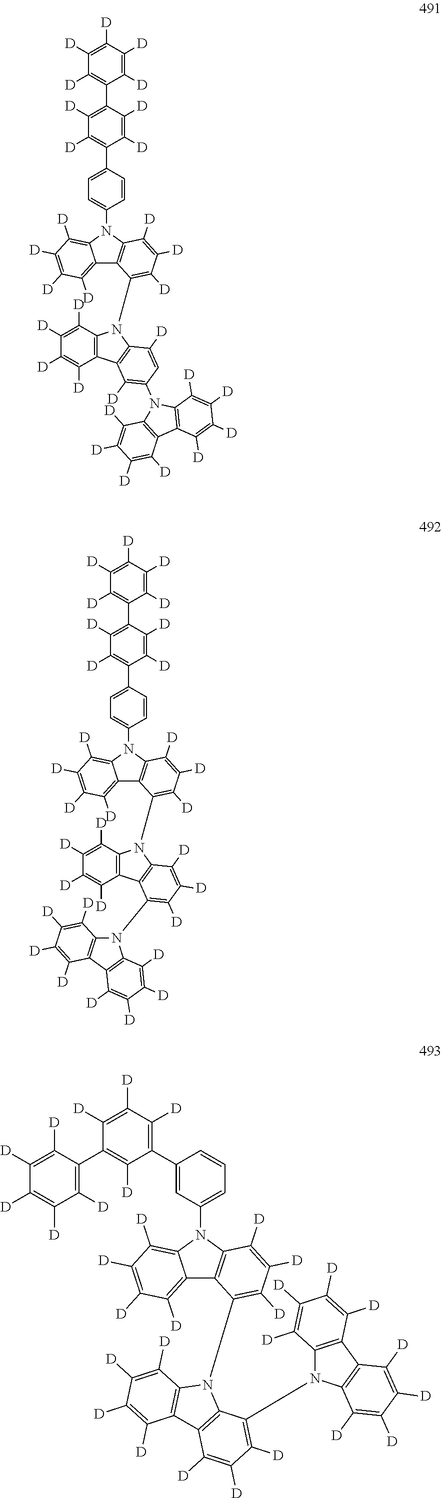

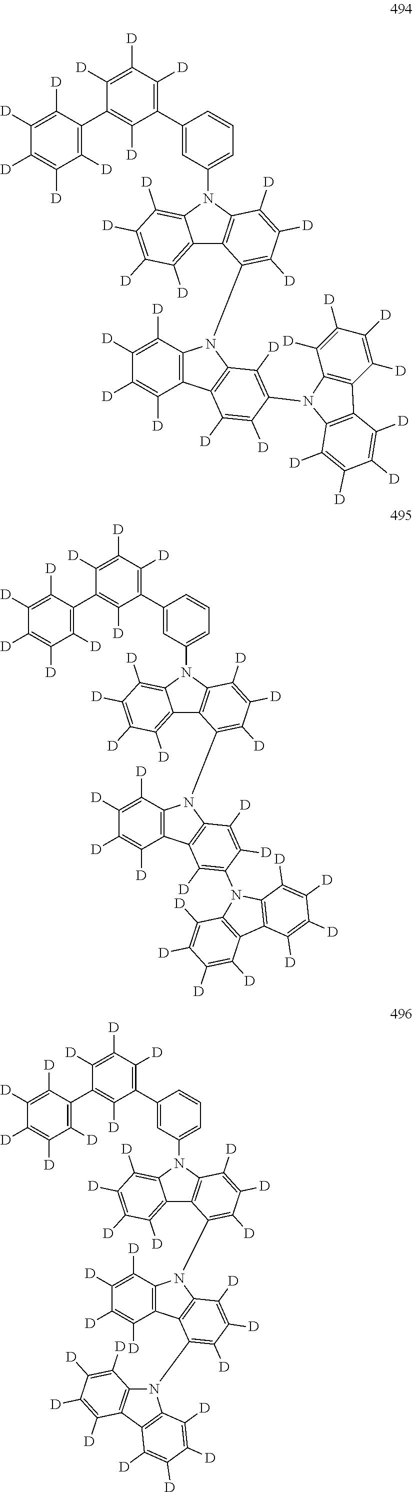

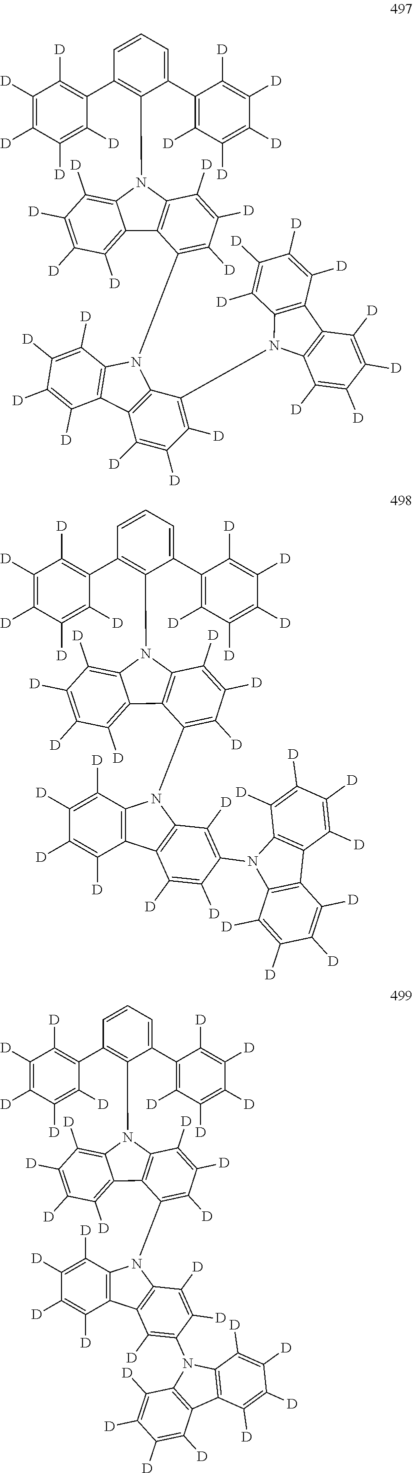

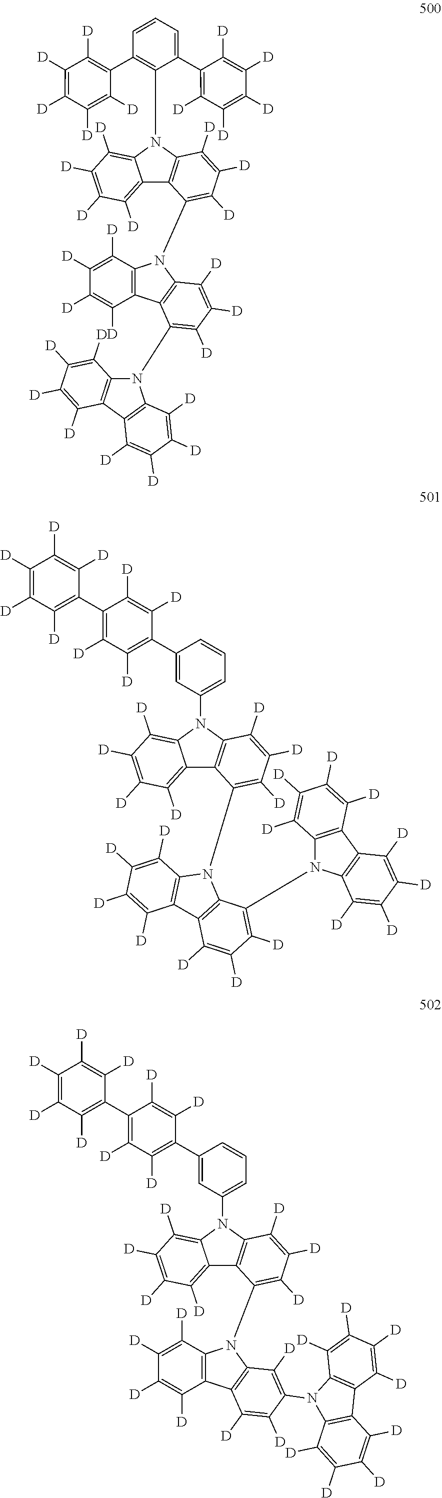

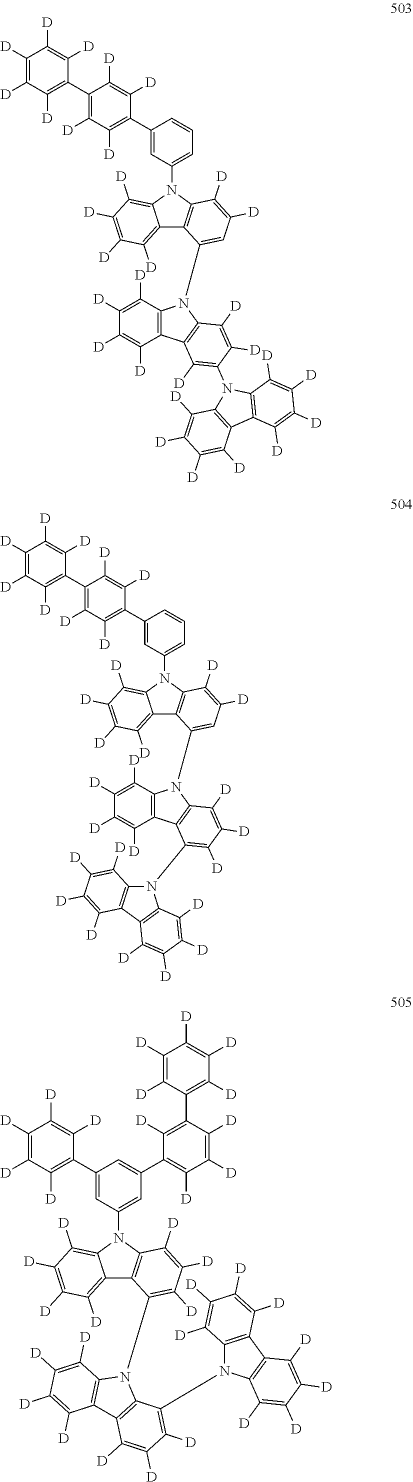

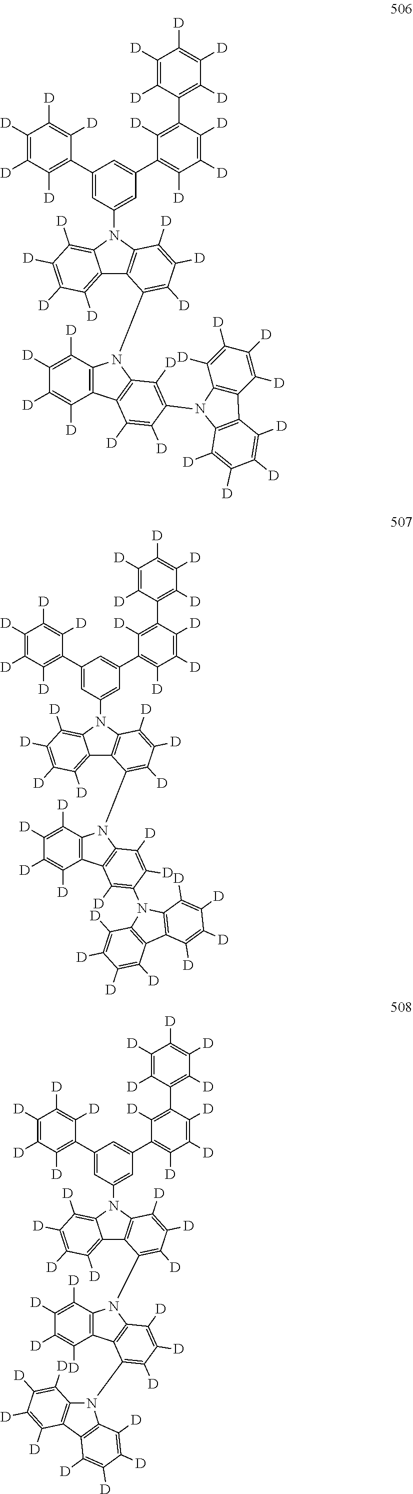

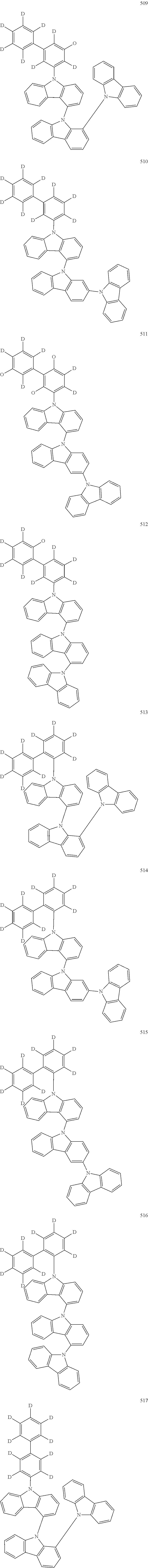

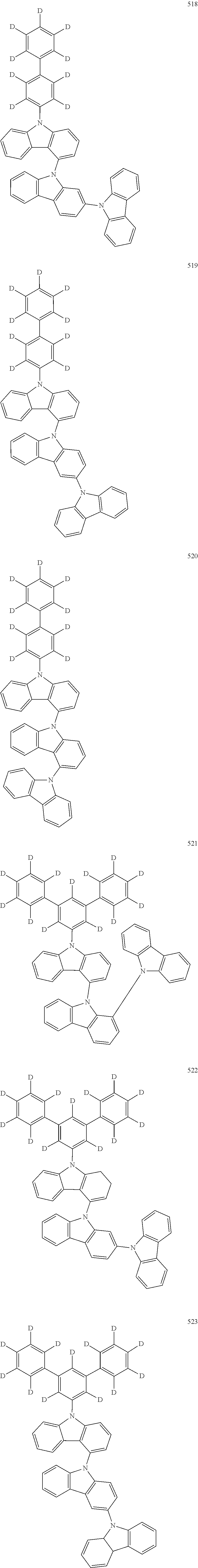

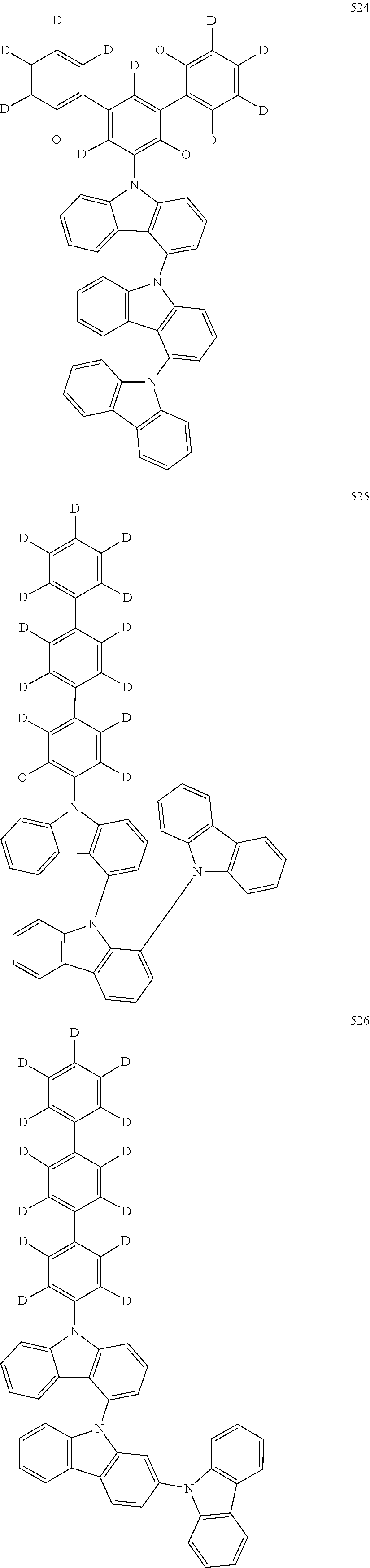

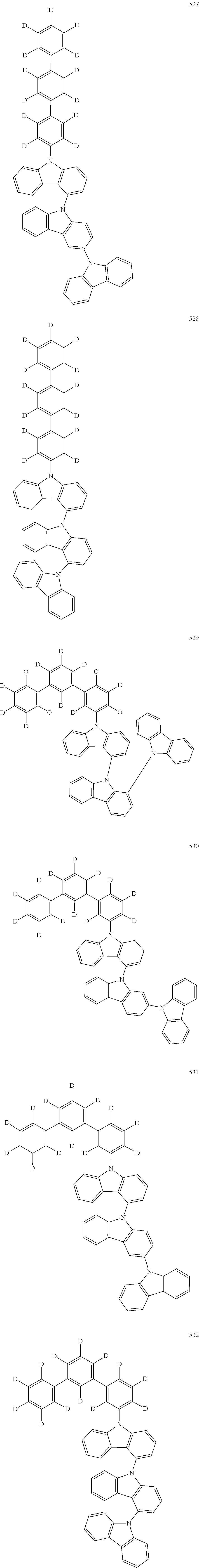

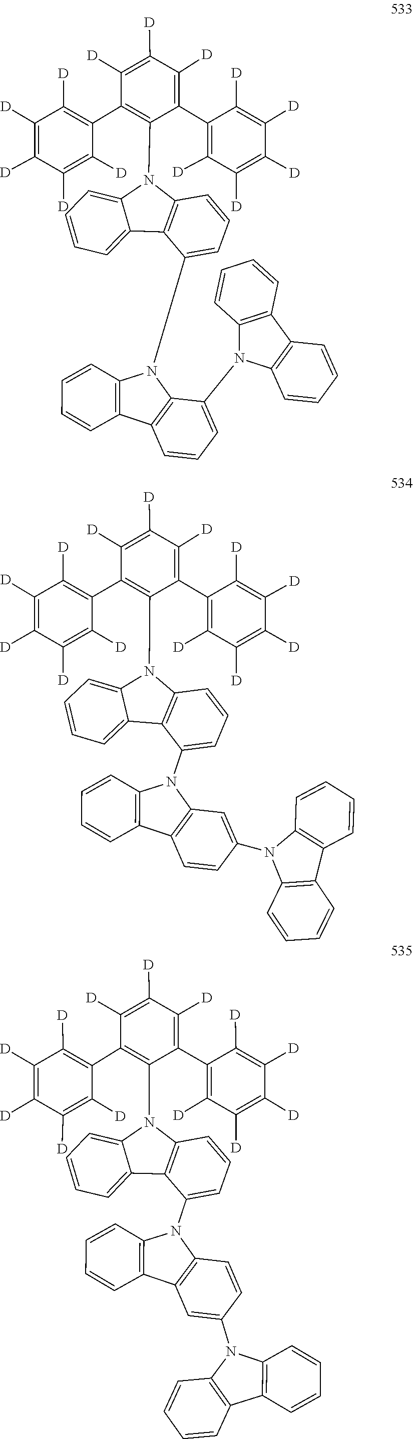

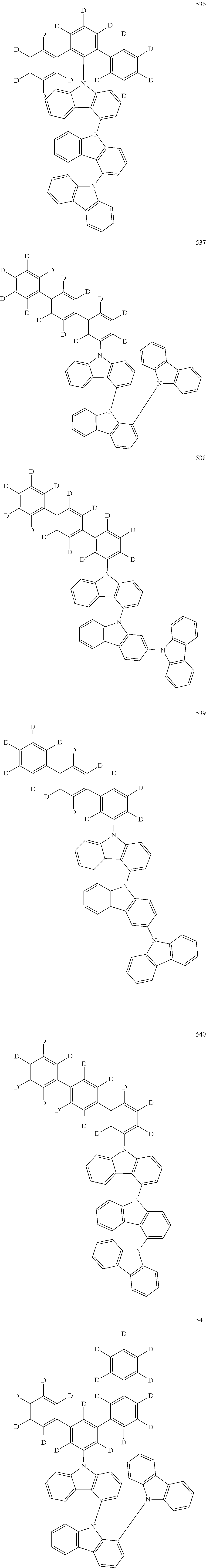

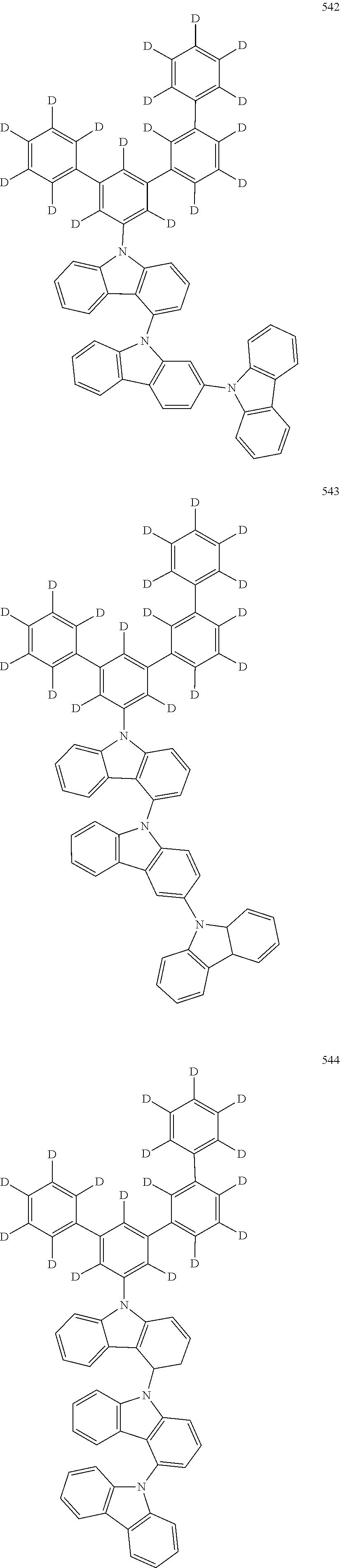

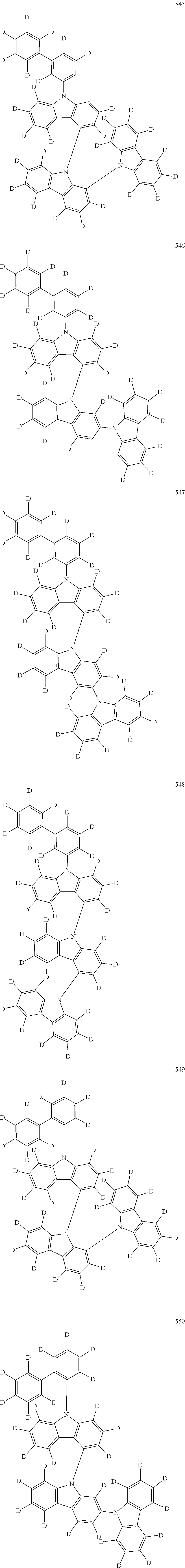

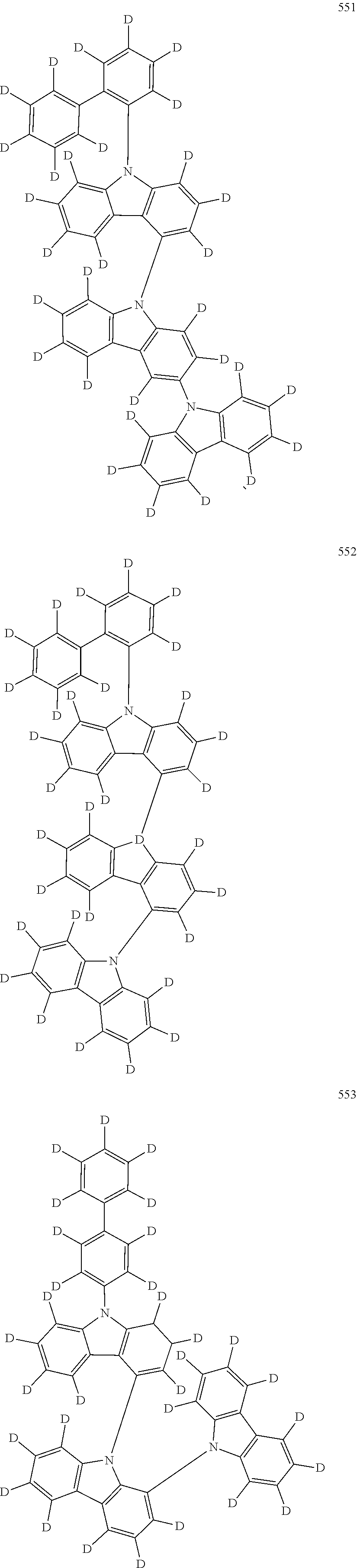

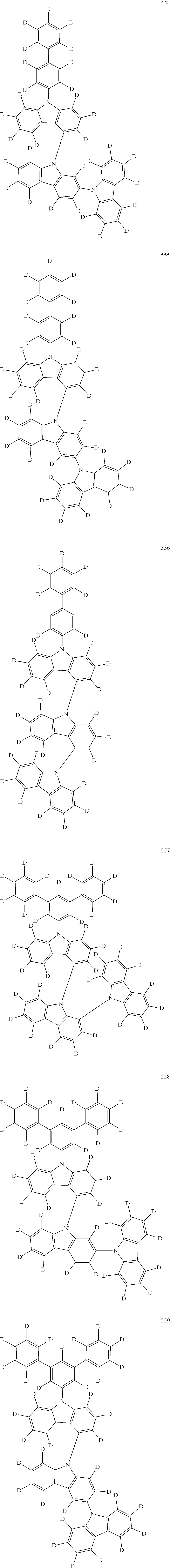

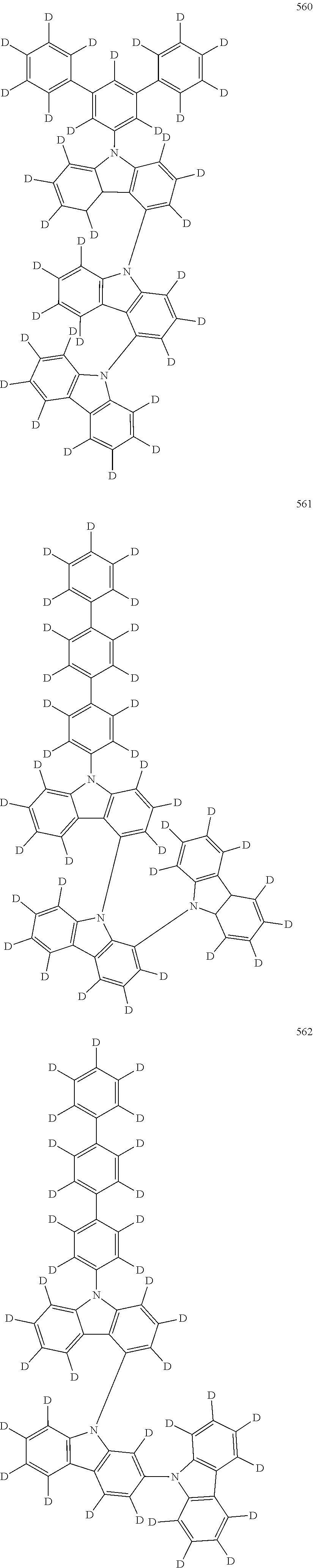

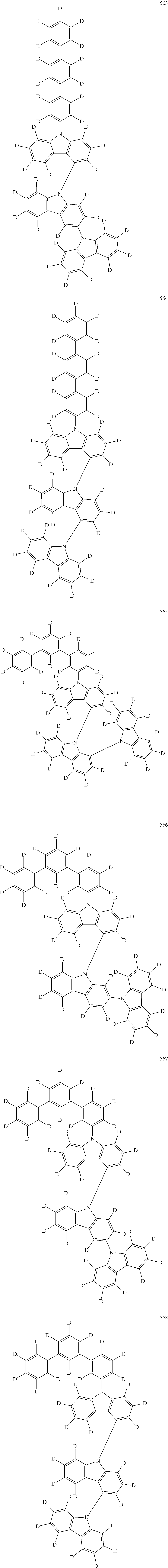

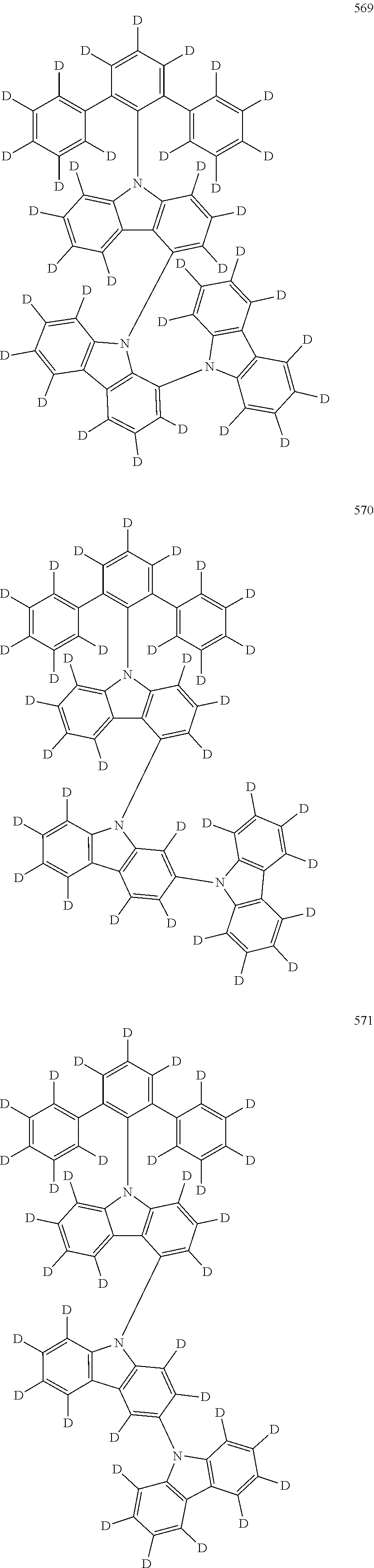

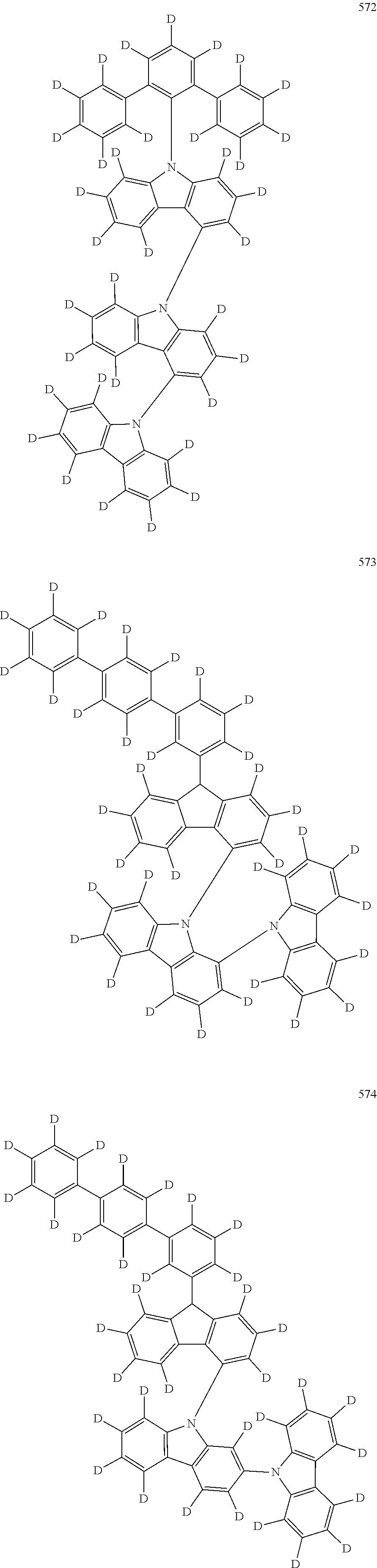

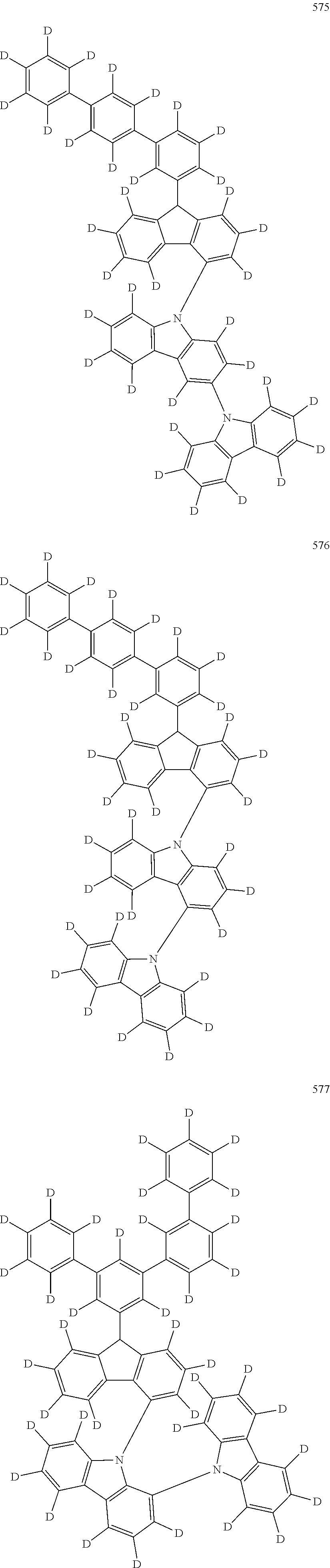

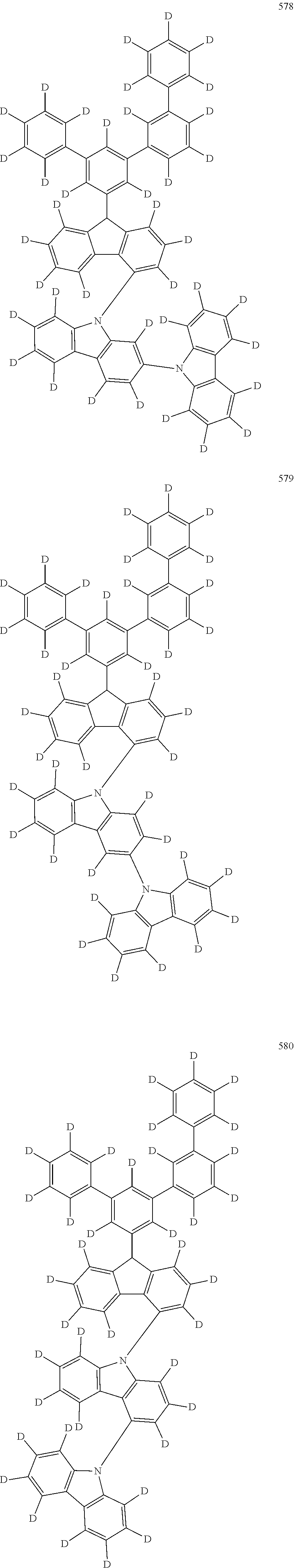

In the heterocyclic compound represented by Formula 1, a first benzene group, a second benzene group, a first carbazole group, and a second carbazole group are sequentially bonded through a single bond, and moreover, a benzene group and a carbazole group may be further included, wherein “N” in the first carbazole group is bonded to the benzene group, and “N” in the second carbazole group is bonded to the first carbazole group (see, e.g., Formula 1′). Accordingly, the heterocyclic compound represented by Formula 1 may have a relatively high triplet (Ti) energy level.

In addition, the heterocyclic compound represented by Formula 1 includes at least one deuterium. A distance of the carbon-deuterium (C-D) bond is shorter than a distance of the carbon-hydrogen (C—H) bond, the bond energy is high, and the frequency is large so that the range of structural fluctuation is small, and thus the heterocyclic compound may have structural stability. In addition, due to the kinetic isotope effect, the heterocyclic compound may have hole stability in structures related to hole movement.

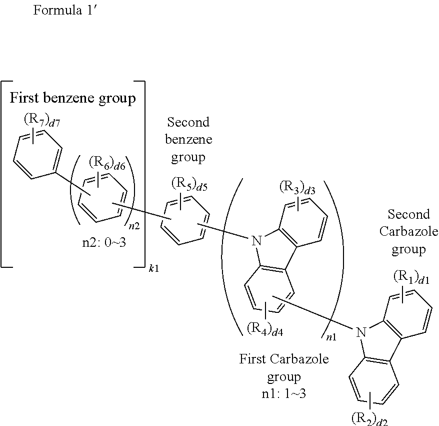

In one or more embodiments, the heterocyclic compound represented by Formula 1 may have a triplet (Ti) energy level of greater than or equal to about 2.8 eV, or about 2.8 eV to about 3.5 eV (e.g., see Table 1).

In one or more embodiments, the heterocyclic compound represented by Formula 1 may have an absolute value of a highest occupied molecular orbital (HOMO) energy level of less than or equal to about 5.2 eV, or about 4.8 eV to about 5.2 eV or about 5.0 eV to about 5.2 eV (e.g., see Table 1).

The triplet (Ti) energy level and the HOMO energy level may be evaluated based on the density functional theory (DFT).

For example, regarding Compounds 331, 339 and 183 belonging to the heterocyclic compound represented by Formula 1, the HOMO energy level, lowest unoccupied molecular orbital (LUMO) energy level, singlet (Si) energy level, triplet (Ti) energy level were calculated using a DFT method of the Gaussian 09 program with the molecular structure optimized at the B3LYP/6-31 G(d,p) levels, and results thereof are shown in Table 1.

TABLE 1

| Compound | HOMO | LUMO | S₁ | T₁ |
|---|---|---|---|---|
| No. | (eV) | (eV) | (eV) | (eV) |
| 331 | −5.039 | −1.167 | 3.757 | 3.252 |
| 339 | −5.042 | −1.190 | 3.758 | 3.255 |
| 183 | −5.147 | −1.074 | 3.886 | 3.207 |

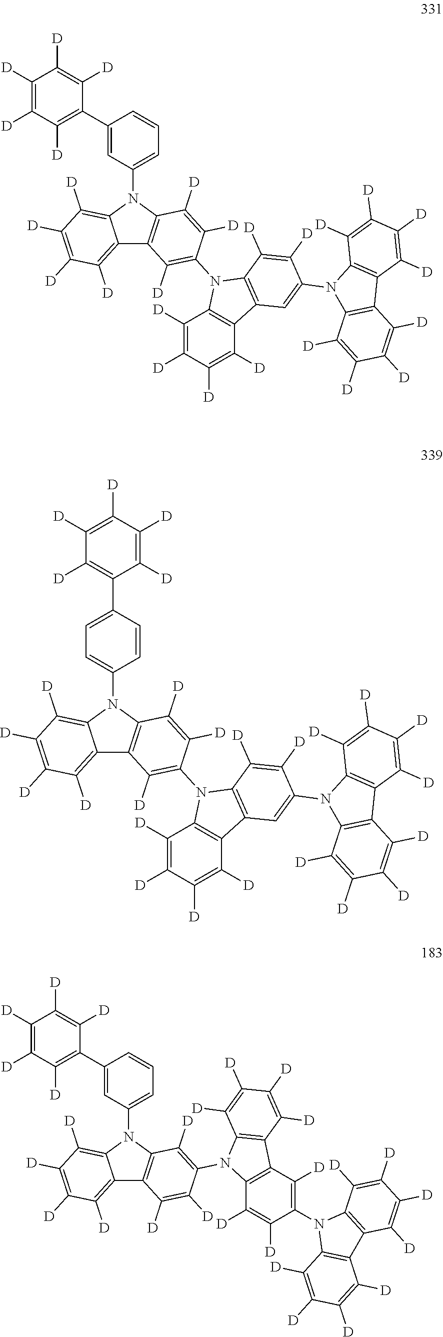

The method of synthesizing the heterocyclic compound represented by Formula 1 may be recognized by those skilled in the art and with reference to the Synthesis Examples described herein.

According to another aspect, an organic light-emitting device includes a first electrode; a second electrode; and an organic layer arranged between the first electrode and the second electrode, wherein the organic layer includes an emission layer, and wherein the organic layer further includes at least one of the heterocyclic compounds represented by Formula 1.

When the organic light-emitting device includes such an organic layer including at least one of the aforementioned heterocyclic compounds represented by Formula 1, long lifespan characteristics or the like may be achieved.

The first electrode may be an anode, which is a hole injection electrode, and the second electrode may be a cathode, which is an electron injection electrode; or the first electrode may be a cathode, which is an electron injection electrode, and the second electrode may be an anode, which is a hole injection electrode.

For example, in the organic light-emitting device, the first electrode may be an anode, the second electrode may be a cathode, and the organic layer may further include a hole transport region arranged between the first electrode and the emission layer, and an electron transport region arranged between the emission layer and the second electrode, wherein the hole transport region may include a hole injection layer, a hole transport layer, an electron blocking layer, an auxiliary layer, or a combination thereof, and the electron transport region may include a buffer layer, a hole blocking layer, an electron transport layer, an electron injection layer, or a combination thereof.

The heterocyclic compound represented by Formula 1 may be used between a pair of electrodes of an organic light-emitting device. In one or more embodiments, at least one of the heterocyclic compounds represented by Formula 1 may be included in the emission layer. In this regard, the heterocyclic compound may act as a host, and the emission layer may further include a dopant (that is, the amount of the at least one of the heterocyclic compound represented by Formula 1 in the emission layer may be greater than an amount of the dopant in the emission layer, based on weight). The emission layer may emit, for example, a blue light.

In one or more embodiments, the heterocyclic compound may be included in the hole transport region. For example, the heterocyclic compound may be included in the auxiliary layer of the hole transport region.

In one or more embodiments, the heterocyclic compound may be included in the electron transport region. For example, the heterocyclic compound may be included in the buffer layer of the electron transport region.

In one or more embodiments, the emission layer may include a host and a dopant, and the heterocyclic compound may be included in the host. In one or more embodiments, the amount (weight) of the host in the emission layer may be greater than the amount (weight) of the dopant in the emission layer.

In one or more embodiments, the emission layer may emit a blue light, but embodiments are not limited thereto.

The dopant may be a fluorescent dopant, a phosphorescent dopant, or a combination thereof. In one or more embodiments, the dopant may be a phosphorescent dopant.

In one or more embodiments, the emission layer may include a host, a fluorescent dopant, and a phosphorescent dopant, and the host may include at least one of the heterocyclic compounds represented by Formula 1. In this regard, the phosphorescent dopant may be a sensitizer compound that is used together with a fluorescent dopant to transfer excitons to the fluorescent dopant.

The expression that an “(organic layer) includes at least one of the heterocyclic compounds of Formula 1” as used herein may be construed as meaning that the “(organic layer) may include one heterocyclic compound of Formula 1 or two or more different heterocyclic compounds of Formula 1”.

In one or more embodiments, the organic layer may include only Compound 1 as the at least one of the heterocyclic compounds of Formula 1. In this embodiment, Compound 1 may be included in the emission layer of the organic light-emitting device. In one or more embodiments, the organic layer may include Compounds 1 and 2 as the at least one of the heterocyclic compounds of Formula 1. In this embodiment, Compound 1 and Compound 2 may exist in an identical layer (for example, Compound 1 and Compound 2 all may exist in an emission layer).

The term “organic layer” as used herein refers to a single layer and/or a plurality of layers that are located between the first electrode and the second electrode of the organic light-emitting device. The “organic layer” may include, in addition to an organic compound, an organometallic complex including a metal.

### DESCRIPTION OF THE FIGURE

The FIGURE is a schematic cross-sectional view of an organic light-emitting device 10 according to one or more embodiments. Hereinafter, the structure and manufacturing method of the organic light-emitting device 10 according to one or more embodiments of the present disclosure will be described in connection with the FIGURE, but embodiments are not limited thereto.

In the FIGURE, an organic light-emitting device 10 includes a first electrode 11, a second electrode 19 facing the first electrode 11, and an organic layer 10A arranged between the first electrode 11 and the second electrode 19.

In the FIGURE, the organic layer 15 may include an emission layer 15, a hole transport region 12 is between the first electrode 11 and an emission layer 15, and an electron transport region 17 is between the emission layer 15 and the second electrode 19.

A substrate may be additionally disposed under the first electrode 11 or on the second electrode 19. The substrate may be a conventional substrate used in organic light-emitting devices, for example, a glass substrate or a transparent plastic substrate, each having excellent mechanical strength, thermal stability, transparency, surface smoothness, ease of handling, and/or water repellency.

### First Electrode 11

The first electrode 11 may be formed by, for example, depositing or sputtering, onto the substrate, a material for forming the first electrode 11. The first electrode 11 may be an anode. The material for forming the first electrode 11 may include materials with a high work function to facilitate hole injection.

The first electrode 11 may be a reflective electrode, a semi-transmissive electrode, or a transmissive electrode. In one or more embodiments, when the first electrode 11 may be a transmissive electrode, the material for forming the first electrode 11 may include indium tin oxide (ITO), indium zinc oxide (IZO), tin oxide (SnO₂), zinc oxide (ZnO), or a combination thereof. In one or more embodiments, when the first electrode 11 may be a semi-transmissive electrode or a reflective electrode, the material for forming the first electrode 11 may include magnesium (Mg), silver (Ag), aluminum (Al), aluminum-lithium (Al—Li), calcium (Ca), magnesium-indium (Mg—In), magnesium-silver (Mg—Ag), or a combination thereof.

The first electrode 11 may have a single-layer structure or a multi-layer structure including a plurality of layers.

### Emission Layer 15

A thickness of the emission layer 15 may be about 100 an angstroms (Å) to about 1,000 Å, for example, about 200 Å to about 600 Å. Without wishing to be bound to theory, when the thickness of the emission layer 15 is within these ranges, excellent luminescence characteristics may be obtained without a substantial increase in driving voltage.

### Host in Emission Layer 15

A host in the emission layer 15 may include at least one of the heterocyclic compounds represented by Formula 1.

In addition to the heterocyclic compound represented by Formula 1 (for example, refer to the first host in Examples described herein), an arbitrary host (for example, refer to the second host in the Examples) may be further included. Hereinafter, a host that may be included in the emission layer 15 in addition to at least one of the heterocyclic compounds represented by Formula 1 will be described herein.

The host may not include a transition metal.

The host may be one kind of compound, or a mixture of two or more different types of compounds.

In one or more embodiments, the host may include at least one of a bipolar host, an electron-transporting host, or a hole-transporting host. The bipolar host, the electron-transporting host, and the hole-transporting host may be different from each other.

The electron-transporting host may include at least one electron-transporting group.

The hole-transporting host may not include an electron-transporting group.

The term “electron-transporting group” as used herein may include a cyano group, a π electron-deficient nitrogen-containing C₁-C₆₀ cyclic group, a group represented by one of the following formulae, or a combination thereof:

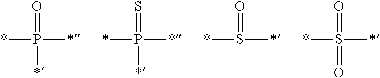

wherein, in the formulae above, *, *′, and *″ each indicate a binding site to a neighboring atom.

In one or more embodiments, the electron-transporting host may include at least one of a cyano group and a r-electron deficient nitrogen-containing C₁-C₆₀ cyclic group.

In one or more embodiments, the electron-transporting host may include at least one cyano group.

In one or more embodiments, the electron-transporting host may include at least one cyano group and a π electron deficient nitrogen-containing C₁-C₆₀ cyclic group.

In one or more embodiments, the host may include a bipolar host.

In one or more embodiments, the host may include an electron-transporting host.

In one or more embodiments, the host may include a hole-transporting host.

In one or more embodiments, the hole-transporting host may not include 1,3-bis(9-carbazolyl)benzene (mCP), tris(4-carbazoyl-9-ylphenyl)amine (TCTA), 4,4′-bis(N-carbazolyl)-1,1′-biphenyl (CBP), 3,3-bis(carbazol-9-yl)biphenyl (mCBP), N,N′-di(1-naphthyl)-N,N′-diphenyl-(1,1′-biphenyl)-4,4′-diamine (NPB), 4,4′,4″-tris[phenyl(m-tolyl)amino]triphenylamine (m-MTDATA), or N,N′-bis(3-methylphenyl)-N,N′-diphenylbenzidine (TPD).

In one or more embodiments,
the host may include an electron-transporting host and a hole-transporting host,
the electron-transporting host may include at least one π electron-rich C₃-C₆₀ ring group and at least one electron-transporting group,
the hole-transporting host may not include at least one π electron-rich C₃-C₆₀ ring group, and may not include an electron-transporting group, and
the electron-transporting group may include a cyano group, a π electron deficient nitrogen-containing C₁-C₆₀ cyclic group, or a combination thereof.

In one or more embodiments, the electron-transporting host may include i) at least one of a cyano group, a pyrimidine group, a pyrazine group, or a triazine group, and ii) at least one of a triphenylene group or a carbazole group.

In one or more embodiments, the hole-transporting host may include at least one carbazole group.

In one or more embodiments, the electron-transporting host may include a compound represented by Formula E-1, and
the hole-transporting host may include a compound represented by Formula H-1:

[Ar₃₀₁]_{xb11}-[(L₃₀₁)_{xb1}-R₃₀₁]_{xb21} Formula E-1

wherein, in Formula E-1,
Ar₃₀₁ may be a C₅-C₆₀ carbocyclic group unsubstituted or substituted with at least one R₃₀₁ₐ, or a C₁-C₆₀ heterocyclic group unsubstituted or substituted with at least one R₃₀₁ₐ,
xb11 may be 1, 2, or 3,
L₃₀₁ may each independently be a single bond, a group represented by one of the following formulae, a C₅-C₆₀ carbocyclic group unsubstituted or substituted with at least one R₃₀₁ₐ, or a C₁-C₆₀ heterocyclic group unsubstituted or substituted with at least one R₃₀₁ₐ, and
*, *′, and *″ in the following formulae each indicate a binding site to a neighboring atom,

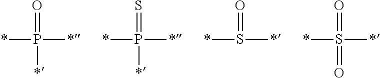

xb1 may be an integer from 1 to 5,
R₃₀₁ₐ and R₃₀₁ may each independently be hydrogen, deuterium, —F, —Cl, —Br, —I, —SF₅, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₁-C₆₀ alkylthio group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀heterocycloalkenyl group, a substituted or unsubstituted C₆-C₆₀ aryl group, a substituted or unsubstituted C₇-C₆₀ alkyl aryl group, a substituted or unsubstituted C₇-C₆₀ aryl alkyl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted C₂-C₆₀ alkyl heteroaryl group, a substituted or unsubstituted C₂-C₆₀ heteroaryl alkyl group, a substituted or unsubstituted C₁-C₆₀ heteroaryloxy group, a substituted or unsubstituted C₁-C₆₀ heteroarylthio group, a substituted or unsubstituted monovalent non-aromatic condensed polycyclic group, a substituted or unsubstituted monovalent non-aromatic condensed heteropolycyclic group, —Si(Q₃₀₁)(Q₃₀₂)(Q₃₀₃), —N(Q₃₀₁)(Q₃₀₂), —B(Q₃₀₁)(Q₃₀₂), —C(═O)(Q₃₀₁), —S(═O)₂(Q₃₀₁), —S(═O)(Q₃₀₁), —P(═O)(Q₃₀₁)(Q₃₀₂), —P(Q₃₀₁)(Q₃₀₂), or —P(═S)(Q₃₀₁)(Q₃₀₂),
xb21 may be an integer from 1 to 5,
Q₃₀₁ to Q₃₀₃ may each independently be a C₁-C₁₀ alkyl group, a C₁-C₁₀ alkoxy group, a C₁-C₆₀ alkylthio group, a phenyl group, a biphenyl group, a terphenyl group, or a naphthyl group, and
at least one of Conditions 1″ to 3″ may be satisfied:
Condition 1″
Ar₃₀₁, L₃₀₁, and R₃₀₁ in Formula E-1 may each independently include a π electron-deficient nitrogen-containing C₁-C₆₀ cyclic group.
Condition 2″
wherein, in Formula E-1, L₃₀₁ may be a group represented by one of the following Formulae:

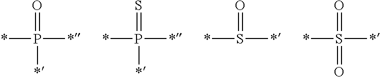

Condition 3″
R₃₀₁ in Formula E-1 is a cyano group, —S(═O)₂(Q₃₀₁), —S(═O)(Q₃₀₁), —P(═O)(Q₃₀₁)(Q₃₀₂), —P(Q₃₀₁)(Q₃₀₂), or —P(═S)(Q₃₀₁)(Q₃₀₂)

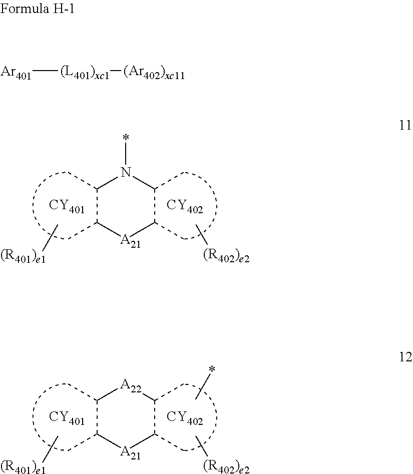

wherein, in Formulae H-1, 11, and 12,
L₄₀₁ may be:
a single bond; or
a π electron-rich C₃-C₆₀ cyclic group, unsubstituted or substituted with at least one of deuterium, a C₁-C₁₀ alkyl group, a C₁-C₁₀ alkoxy group, a C₁-C₆₀ alkylthio group, a phenyl group, a naphthyl group, a fluorenyl group, a carbazolyl group, a dibenzofuranyl group, a dibenzothiophenyl group, a triphenylenyl group, a biphenyl group, a terphenyl group, a tetraphenyl group, —Si(Q₄₀₁)(Q₄₀₂)(Q₄₀₃), or a combination thereof,
xc1 may be an integer from 1 to 10, wherein, when xc1 is 2 or greater, two or more of L₄₀₁ may be identical to or different from each other,
Ar₄₀₁ may be a group represented by Formula 11 or 12,
Ar₄₀₂ may be:
a group represented by Formula 11 or 12; or
a π electron-rich C₃-C₆₀ cyclic group (for example, a phenyl group, a naphthyl group, a fluorenyl group, a carbazolyl group, a dibenzofuranyl group, a dibenzothiophenyl group, a biphenyl group, a terphenyl group, a triphenylenyl group, or the like), each unsubstituted or substituted with at least one of deuterium, a C₁-C₁₀ alkyl group, a C₁-C₁₀ alkoxy group, a C₁-C₆₀ alkylthio group, a phenyl group, a naphthyl group, a fluorenyl group, a carbazolyl group, a dibenzofuranyl group, a dibenzothiophenyl group, a triphenylenyl group, a biphenyl group, a terphenyl group, a tetraphenyl group, or a combination thereof,
xc11 may be an integer from 1 to 10, wherein, when xc11 is 2 or greater, two or more of Ar₄₀₂ may be identical to or different from each other,
ring CY₄₀₁ and ring CY₄₀₂ may each independently be a π electron-rich C₃-C₆₀ cyclic group (for example, a benzene group, a naphthalene group, a fluorene group, a carbazole group, a benzocarbazole group, an indolocarbazole group, a dibenzofuran group, a dibenzothiophene group, a dibenzosilole group, a benzonaphthofuran group, a benzonaphthothiophene group, a benzonaphthosilole group, or the like),
A₂₁ may be a single bond, O, S, N(R₄₁₁), C(R₄₁₁)(R₄₁₂), or Si(R₄₁₁)(R₄₁₂),
A₂₂ may be a single bond, O, S, N(R₄₁₁), C(R₄₁₁)(R₄₁₂), or Si(R₄₁₁)(R₄₁₂),
at least one of A₂₁ and A₂₂ in Formula 12 may not be a single bond,
R₄₀₁, R₄₀₂, R₄₁₁, and R₄₁₂ may each independently be:
hydrogen, deuterium, a C₁-C₂₀ alkyl group, a C₁-C₂₀ alkoxy group, or a C₁-C₂₀ alkylthio group;
a C₁-C₂₀ alkyl group, a C₁-C₂₀ alkoxy group, or a C₁-C₂₀ alkylthio group, each substituted with at least one of deuterium, a phenyl group, a naphthyl group, a fluorenyl group, a carbazolyl group, a dibenzofuranyl group, a dibenzothiophenyl group, or a combination thereof;
π electron-rich C₃-C₆₀ cyclic group, unsubstituted or substituted with at least one of deuterium, a C₁-C₂₀ alkyl group, a C₁-C₂₀ alkoxy group, a C₁-C₂₀ alkylthio group, a phenyl group, a naphthyl group, a fluorenyl group, a carbazolyl group, a dibenzofuranyl group, a dibenzothiophenyl group, a biphenyl group, or a combination thereof; or
—Si(Q₄₀₄)(Q₄₀₅)(Q₄₀₆),
e1 and e2 may each independently be an integer from 0 to 10,
Q₄₀₁ to Q₄₀₆ may each independently be hydrogen, deuterium, a C₁-C₂₀ alkyl group, a phenyl group, a naphthyl group, a fluorenyl group, a carbazolyl group, a dibenzofuranyl group, a dibenzothiophenyl group, a biphenyl group, a terphenyl group, or a triphenylenyl group, and
* indicates a binding site to a neighboring atom.

In one or more embodiments, Ar₃₀₁ and L₃₀₁ in Formula E-1 may each independently be a benzene group, a naphthalene group, a fluorene group, a spiro-bifluorene group, a benzofluorene group, a dibenzofluorene group, a phenalene group, a phenanthrene group, an anthracene group, a fluoranthene group, a triphenylene group, a pyrene group, a chrysene group, a naphthacene group, a picene group, a perylene group, a pentaphene group, an indenoanthracene group, a dibenzofuran group, a dibenzothiophene group, an imidazole group, a pyrazole group, a thiazole group, an isothiazole group, an oxazole group, an isoxazole group, a pyridine group, a pyrazine group, a pyridazine group, a pyrimidine group, an indazole group, a purine group, a quinoline group, an isoquinoline group, a benzoquinoline group, a phthalazine group, a naphthyridine group, a quinoxaline group, a quinazoline group, a cinnoline group, a phenanthridine group, an acridine group, a phenanthroline group, a phenazine group, a benzimidazole group, an iso-benzothiazole group, a benzoxazole group, a benzoisoxazole group, a triazole group, a tetrazole group, an oxadiazole group, a triazine group, a thiadiazole group, an imidazopyridine group, an imidazopyrimidine group, or an azacarbazole group, each unsubstituted or substituted with at least one of deuterium, —F, —Cl, —Br, —I, —SF₅, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a C₁-C₂₀ alkylthio group, a phenyl group, a biphenyl group, a terphenyl group, a naphthyl group, a cyano group-containing phenyl group, a cyano group-containing biphenyl group, a cyano group-containing terphenyl group, a cyano group-containing naphthyl group, a pyridinyl group, a phenylpyridinyl group, a diphenylpyridinyl group, a biphenylpyridinyl group, a di(biphenyl)pyridinyl group, a pyrazinyl group, a phenylpyrazinyl group, a diphenylpyrazinyl group, a biphenylpyrazinyl group, a di(biphenyl)pyrazinyl group, a pyridazinyl group, a phenylpyridazinyl group, a diphenylpyridazinyl group, a biphenylpyridazinyl group, a di(biphenyl)pyridazinyl group, a pyrimidinyl group, phenylpyrimidinyl group, a diphenylpyrimidinyl group, a biphenylpyrimidinyl group, a di(biphenyl)pyrimidinyl group, a triazinyl group, a phenyltriazinyl group, a diphenyltriazinyl group, a biphenyltriazinyl group, a di(biphenyl)triazinyl group, —Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), —P(═O)(Q₃₁)(Q₃₂), —P(Q₃₁)(Q₃₂), or a combination thereof,
at least one L₃₀₁ may each independently be an imidazole group, a pyrazole group, a thiazole group, an isothiazole group, an oxazole group, an isoxazole group, a pyridine group, a pyrazine group, a pyridazine group, a pyrimidine group, an indazole group, a purine group, a quinoline group, an isoquinoline group, a benzoquinoline group, a phthalazine group, a naphthyridine group, a quinoxaline group, a quinazoline group, a cinnoline group, a phenanthridine group, an acridine group, a phenanthroline group, a phenazine group, a benzimidazole group, an isobenzothiazole group, a benzoxazole group, an isobenzoxazole group, a triazole group, a tetrazole group, an oxadiazole group, a triazine group, a thiadiazole group, an imidazopyridine group, an imidazopyrimidine group, or an azacarbazole group, each unsubstituted or substituted with at least one of deuterium, —F, —Cl, —Br, —I, —SF₅, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a C₁-C₂₀ alkylthio group, a phenyl group, a biphenyl group, a terphenyl group, a naphthyl group, a cyano-containing phenyl group, a cyano-containing a biphenyl group, a cyano-containing terphenyl group, a cyano-containing a naphthyl group, a pyridinyl group, a phenylpyridinyl group, a diphenylpyridinyl group, a biphenylpyridinyl group, a di(biphenyl)pyridinyl group, a pyrazinyl group, a phenylpyrazinyl group, a diphenylpyrazinyl group, a biphenylpyrazinyl group, a di(biphenyl)pyrazinyl group, a pyridazinyl group, a phenylpyridazinyl group, a diphenylpyridazinyl group, a biphenylpyridazinyl group, a di(biphenyl)pyridazinyl group, a pyrimidinyl group, a phenylpyrimidinyl group, a diphenylpyrimidinyl group, a biphenylpyrimidinyl group, a di(biphenyl)pyrimidinyl group, a triazinyl group, a phenyltriazinyl group, a diphenyltriazinyl group, a biphenyltriazinyl group, a di(biphenyl)triazinyl group, —Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), —P(═O)(Q₃₁)(Q₃₂), —P(Q₃₁)(Q₃₂), or a combination thereof,
R₃₀₁ may be hydrogen, deuterium, —F, —Cl, —Br, —I, —SF₅, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a C₁-C₂₀ alkylthio group, a phenyl group, a biphenyl group, a terphenyl group, a tetraphenyl group, a naphthyl group, a cyano group-containing phenyl group, a cyano group-containing biphenyl group, a cyano group-containing terphenyl group, a cyano group-containing tetraphenyl group, a cyano group-containing naphthyl group, a pyridinyl group, a phenylpyridinyl group, a diphenylpyridinyl group, a biphenylpyridinyl group, a di(biphenyl)pyridinyl group, a pyrazinyl group, a phenylpyrazinyl group, a diphenylpyrazinyl group, a biphenylpyrazinyl group, a di(biphenyl)pyrazinyl group, a pyridazinyl group, a phenylpyridazinyl group, a diphenylpyridazinyl group, a biphenylpyridazinyl group, a di(biphenyl)pyridazinyl group, a pyrimidinyl group, a phenylpyrimidinyl group, a diphenylpyrimidinyl group, a biphenylpyrimidinyl group, a di(biphenyl)pyrimidinyl group, a triazinyl group, a phenyltriazinyl group, a diphenyltriazinyl group, a biphenyltriazinyl group, a di(biphenyl)triazinyl group, —Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), —P(═O)(Q₃₁)(Q₃₂), or —P(Q₃₁)(Q₃₂), and
Q₃₁ to Q₃₃ may each independently be a C₁-C₁₀ alkyl group, a C₁-C₁₀ alkoxy group, a C₁-C₁₀ alkylthio group, a phenyl group, a biphenyl group, a terphenyl group, or a naphthyl group.

In one or more embodiments,
Ar₃₀₁ may be a benzene group, a naphthalene group, a fluorene group, a spiro-bifluorene group, a benzofluorene group, a dibenzofluorene group, a phenalene group, a phenanthrene group, an anthracene group, a fluoranthene group, a triphenylene group, a pyrene group, a chrysene group, a naphthacene group, a picene group, a perylene group, a pentaphene group, an indenoanthracene group, a dibenzofuran group, or a dibenzothiophene group, each unsubstituted or substituted with at least one of deuterium, —F, —Cl, —Br, —I, —SF₅, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a C₁-C₂₀ alkylthio group, a phenyl group, a biphenyl group, a terphenyl group, a naphthyl group, a cyano group-containing phenyl group, a cyano group-containing biphenyl group, a cyano group-containing terphenyl group, a cyano group-containing naphthyl group, a pyridinyl group, a phenylpyridinyl group, a diphenylpyridinyl group, a biphenylpyridinyl group, a di(biphenyl)pyridinyl group, a pyrazinyl group, a phenylpyrazinyl group, a diphenylpyrazinyl group, a biphenylpyrazinyl group, a di(biphenyl)pyrazinyl group, a pyridazinyl group, a phenylpyridazinyl group, a diphenylpyridazinyl group, a biphenylpyridazinyl group, a di(biphenyl)pyridazinyl group, a pyrimidinyl group, a phenylpyrimidinyl group, a diphenylpyrimidinyl group, a biphenylpyrimidinyl group, a di(biphenyl)pyrimidinyl group, a triazinyl group, a phenyltriazinyl group, a diphenyltriazinyl group, a biphenyltriazinyl group, a di(biphenyl)triazinyl group, —Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), —P(═O)(Q₃₁)(Q₃₂), —P(Q₃₁)(Q₃₂), or a combination thereof; or
a group represented by one of Formulae 5-1 to 5-3 or 6-1 to 6-33, and
L₃₀₁ may be a group represented by one Formulae 5-1 to 5-3 or Formula 6-1 to 6-33:

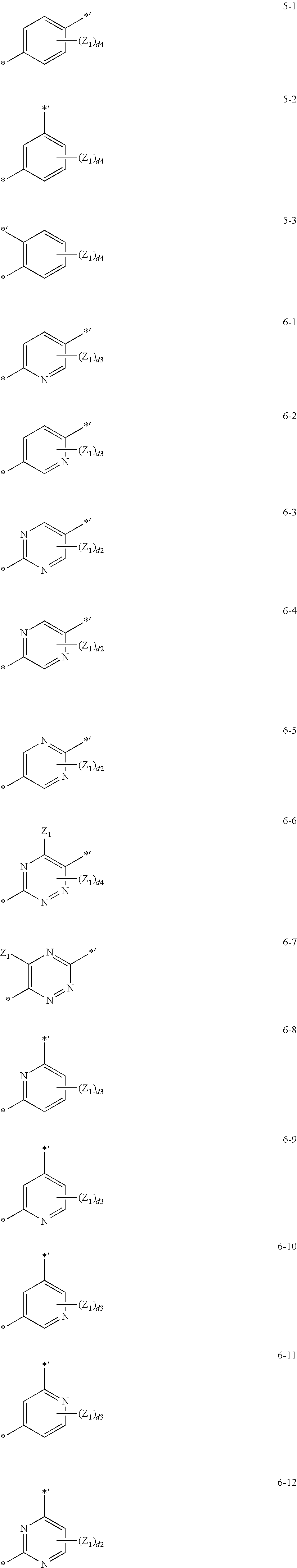

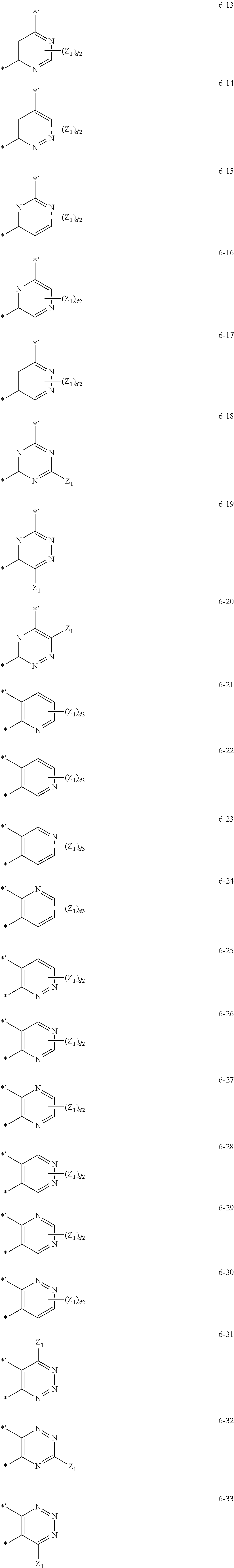

wherein, in Formulae 5-1 to 5-3 and 6-1 to 6-33,
Z₁ may be hydrogen, deuterium, —F, —Cl, —Br, —I, —SF₅, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a C₁-C₂₀ alkylthio group, a phenyl group, a biphenyl group, a terphenyl group, a naphthyl group, a cyano group-containing phenyl group, a cyano group-containing biphenyl group, a cyano group-containing terphenyl group, a cyano group-containing naphthyl group, a pyridinyl group, a phenylpyridinyl group, a diphenylpyridinyl group, a biphenylpyridinyl group, a di(biphenyl)pyridinyl group, a pyrazinyl group, a phenylpyrazinyl group, a diphenylpyrazinyl group, a biphenylpyrazinyl group, a di(biphenyl)pyrazinyl group, a pyridazinyl group, a phenylpyridazinyl group, a diphenylpyridazinyl group, a biphenylpyridazinyl group, a di(biphenyl)pyridazinyl group, a pyrimidinyl group, a phenylpyrimidinyl group, a diphenylpyrimidinyl group, a biphenylpyrimidinyl group, a di(biphenyl)pyrimidinyl group, a triazinyl group, a phenyltriazinyl group, a diphenyltriazinyl group, a biphenyltriazinyl group, a di(biphenyl)triazinyl group, —Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), —P(═O)(Q₃₁)(Q₃₂), or —P(Q₃₁)(Q₃₂),
d4 may be 0, 1, 2, 3, or 4,
d3 may be 0, 1, 2, or 3,
d2 may be 0, 1, or 2, and
* and *′ each indicate a binding site to a neighboring atom.

Q₃₁ to Q₃₃ may each be as described herein.

In one or more embodiments, L₃₀₁ may be a group represented by one of Formulae 5-2, 5-3, or 6-8 to 6-33.

In one or more embodiments, R₃₀₁ may be a cyano group or a group represented by one of Formulae 7-1 to 7-18, and at least one Ar₄₀₂ may be a group represented by one of Formulae 7-1 to 7-18:

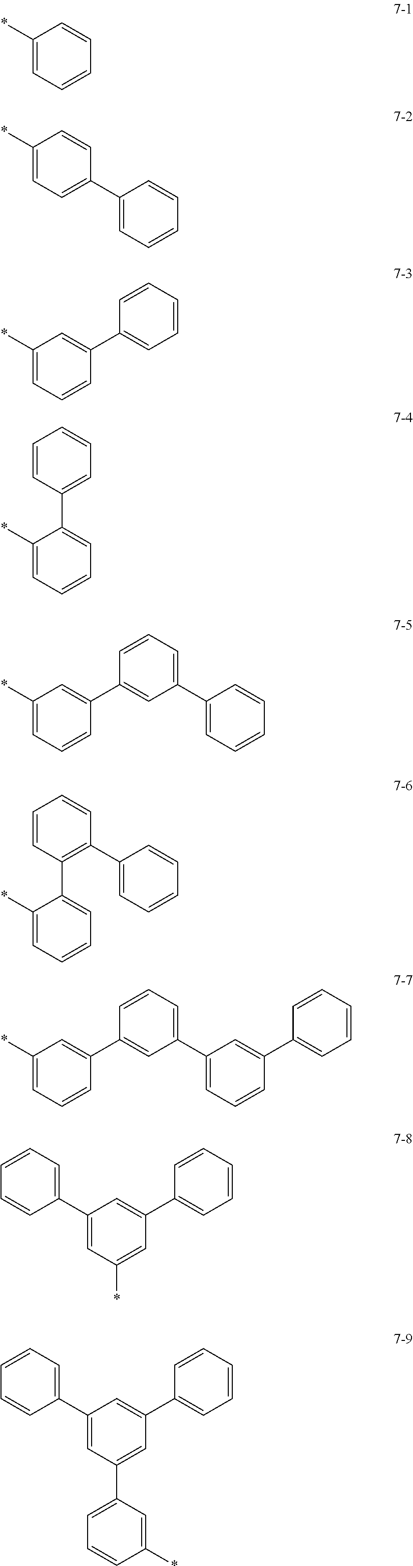

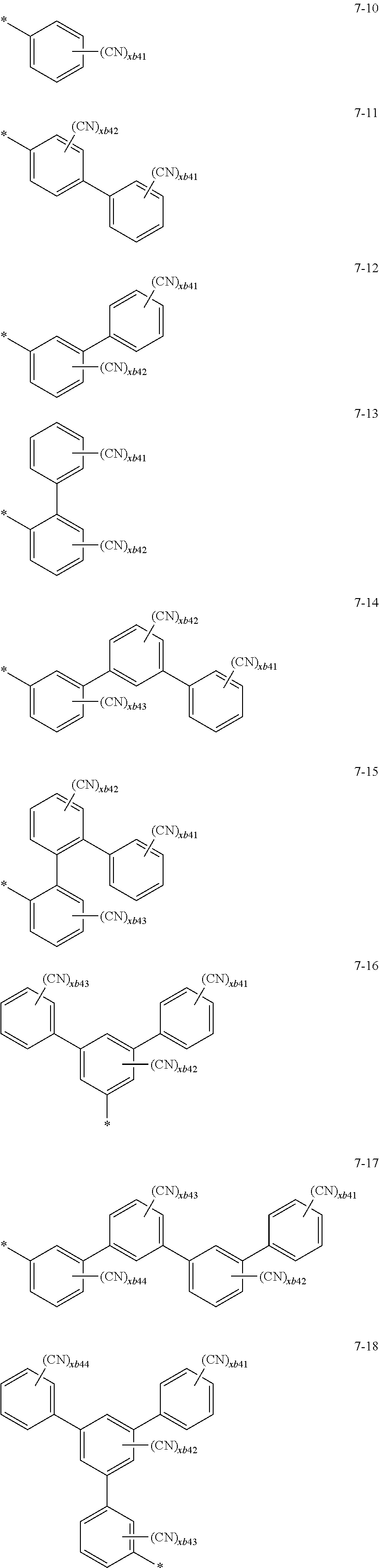

wherein, in Formulae 7-1 to 7-18,
xb41 to xb44 may each be 0, 1, or 2, wherein xb41 in Formula 7-10 may not be 0, xb41+xb42 in Formulae 7-11 to 7-13 may not be 0, xb41+xb42+xb43 in Formulae 7-14 to 7-16 may not be 0, xb41+xb42+xb43+xb44 in Formulae 7-17 and 7-18 may not be 0, and * indicates a binding site to a neighboring atom.

In Formula E-1, two or more of Ar₃₀₁ may be identical to or different from each other, and two or more of L₃₀₁ may be identical to or different from each other. In Formula H-1, two or more of L₄₀₁ may be identical to or different from each other, and two or more of Ar₄₀₂ may be identical to or different from each other.

In one or more embodiments, the electron-transporting host may be represented by Formula E-2, but embodiments are not limited thereto:

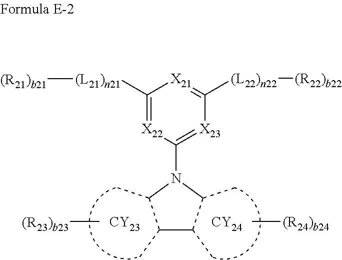

In one or more embodiments, in Formula E-2, ring CY₂₃ and ring CY₂₄ may each independently be a C₅-C₃₀ carbocyclic group or a C₁-C₃₀ heterocyclic group.

In one or more embodiments, ring CY₂₃ and ring CY₂₄ may each independently be a benzene group, a naphthalene group, an anthracene group, a phenanthrene group, a triphenylene group, a pyrene group, a chrysene group, a cyclopentadiene group, a 1,2,3,4-tetrahydronaphthalene group, a thiophene group, a furan group, an indole group, a benzoborole group, a benzophosphole group, an indene group, a benzosilole group, a benzogermole group, a benzothiophene group, a benzoselenophene group, a benzofuran group, a carbazole group, a dibenzoborole group, a dibenzophosphole group, a fluorene group, a dibenzosilole group, a dibenzogermole group, a dibenzothiophene group, a dibenzoselenophene group, a dibenzofuran group, a dibenzothiophene 5-oxide group, a 9H-fluorene-9-one group, a dibenzothiophene 5,5-dioxide group, an azaindole group, an azabenzoborole group, an azabenzophosphole group, an azaindene group, an azabenzosilole group, an azabenzogermole group, an azabenzothiophene group, an azabenzoselenophene group, an azabenzofuran group, an azacarbazole group, an azadibenzoborole group, an azadibenzophosphole group, an azafluorene group, an azadibenzosilole group, an azadibenzogermole group, an azadibenzothiophene group, an azadibenzoselenophene group, an azadibenzofuran group, an azadibenzothiophene 5-oxide group, an aza-9H-fluorene-9-one group, an azadibenzothiophene 5,5-dioxide group, a pyridine group, a pyrimidine group, a pyrazine group, a pyridazine group, a triazine group, a quinoline group, an isoquinoline group, a quinoxaline group, a quinazoline group, a phenanthroline group, a pyrrole group, a pyrazole group, an imidazole group, a triazole group, an oxazole group, an isoxazole group, a thiazole group, an isothiazole group, an oxadiazole group, a thiadiazole group, a benzopyrazole group, a benzimidazole group, a benzoxazole group, a benzothiazole group, a benzoxadiazole group, a benzothiadiazole group, a 5,6,7,8-tetrahydroisoquinoline group, or a 5,6,7,8-tetrahydroquinoline group, each unsubstituted or substituted with at least one R₁₀ₐ. R₁₀ₐ may be as described herein in connection with R₂₃.

In one or more embodiments, ring CY₂₃ and ring CY₂₄ may each independently be a benzene group, a naphthalene group, or a pyridine group.

In one or more embodiments, in Formula E-2, X₂₁ may be N or C(R₂₁ₐ), X₂₂ may be N or C(R₂₂ₐ), and X₂₃ may be N or C(R₂₃ₐ).

In one or more embodiments, in Formula E-2, at least one of X₂₁ to X₂₃ may be N.

For example, one of X₂₁ to X₂₃ may be N, two of X₂₁ to X₂₃ may each be N, and X₂₁ to X₂₃ may each be N.

In one or more embodiments, in Formula E-2, L₂₁ and L₂₂ may each independently be a single bond, a substituted or unsubstituted C₃-C₆₀ carbocyclic group, or a substituted or unsubstituted C₁-C₆₀ heterocyclic group.

For example, L₂₁ and L₂₂ may each independently be:
a single bond; or
a benzene group, a naphthalene group, an anthracene group, a phenanthrene group, a triphenylene group, a pyrene group, a chrysene group, a cyclopentadiene group, a 1,2,3,4-tetrahydronaphthalene group, a thiophene group, a furan group, an indole group, a benzoborole group, a benzophosphole group, an indene group, a benzosilole group, a benzogermole group, a benzothiophene group, a benzoselenophene group, a benzofuran group, a carbazole group, a dibenzoborole group, a dibenzophosphole group, a fluorene group, a dibenzosilole group, a dibenzogermole group, a dibenzothiophene group, a dibenzoselenophene group, a dibenzofuran group, a dibenzothiophene 5-oxide group, a 9H-a fluorene-9-one group, a dibenzothiophene 5,5-dioxide group, an azaindole group, an azabenzoborole group, an azabenzophosphole group, an azaindene group, an azabenzosilole group, an azabenzogermole group, an azabenzothiophene group, an azabenzoselenophene group, an azabenzofuran group, an azacarbazole group, an azadibenzoborole group, an azadibenzophosphole group, an azafluorene group, an azadibenzosilole group, an azadibenzogermole group, an azadibenzothiophene group, an azadibenzoselenophene group, an azadibenzofuran group, an azadibenzothiophene 5-oxide group, an aza-9H-fluoren-9-one group, an azadibenzothiophene 5,5-dioxide group, a pyridine group, a pyrimidine group, a pyrazine group, a pyridazine group, a triazine group, a quinoline group, an isoquinoline group, a quinoxaline group, a quinazoline group, a phenanthroline group, a pyrrole group, a pyrazole group, an imidazole group, a triazole group, an oxazole group, an isoxazole group, a thiazole group, an isothiazole group, an oxadiazole group, a thiadiazole group, a benzopyrazole group, a benzimidazole group, a benzoxazole group, a benzothiazole group, a benzoxadiazole group, a benzothiadiazole group, a 5,6,7,8-tetrahydroisoquinoline group, or a 5,6,7,8-tetrahydroquinoline group, each unsubstituted or substituted with at least one R₁₀ₐ. R₁₀ₐ may be as described herein in connection with R₂₃.

In one or more embodiments, in Formula E-2, L₂₁ and L₂₂ may each independently be:
a single bond; or
a group represented by one of Formulae L-1 to L-12:

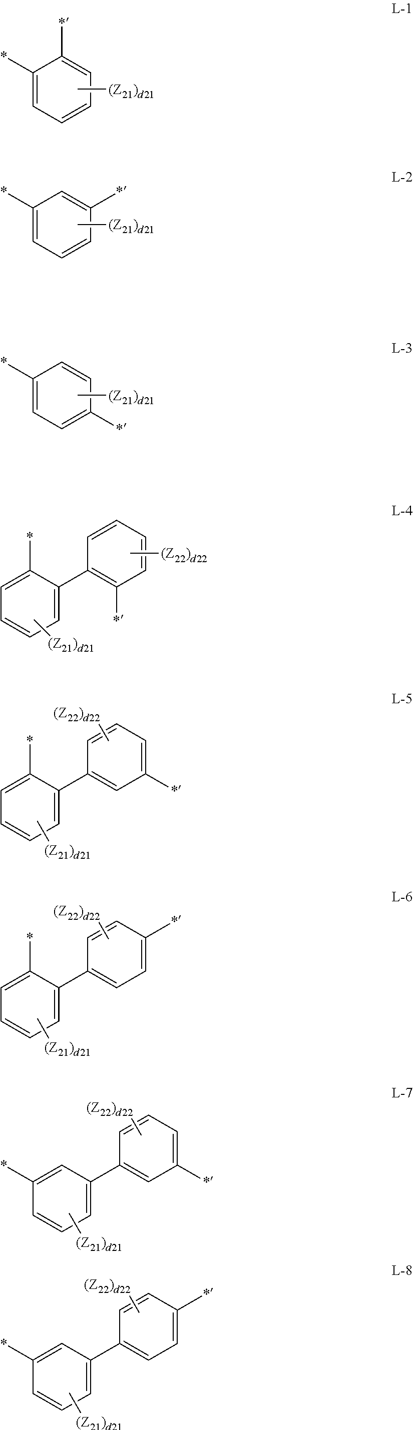

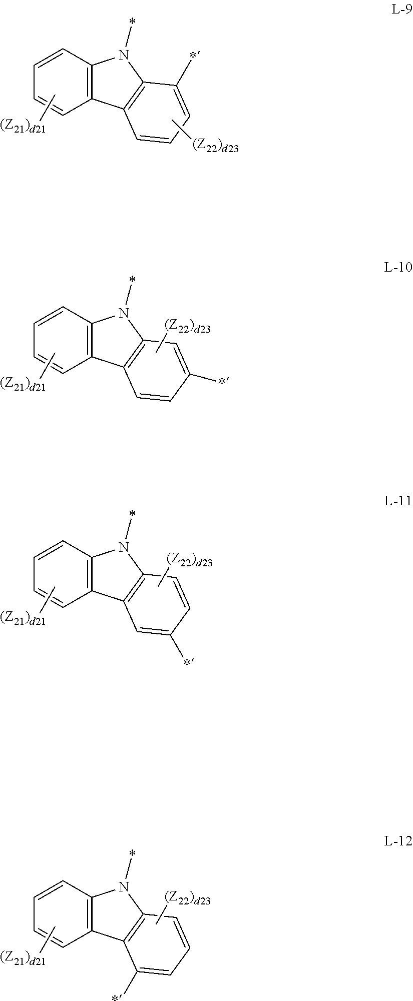

wherein, in Formulae L-1 to L-12,
Z₂₁ and Z₂₂ are each as described herein in connection with R₂₁,
d21 and d22 may each independently be an integer from 0 to 4,
d23 may be an integer from 0 to 3, and
* and *′ each indicate a binding site to a neighboring atom.

In one or more embodiments, in Formula E-2, n21 and n22 each indicate the number of L₂₁(s) and L₂₂(s), respectively, and may each independently be an integer from 0 to 5 (for example, 0, 1, 2, or 3). When n21 is 2 or greater, two or more of L₂₁ may be identical to or different from each other, and when n22 is 2 or greater, two or more of L₂₂ may be identical to or different from each other.

In one or more embodiments, in Formula E-2, R₂₁ to R₂₄, R₂₁ₐ, R₂₂ₐ, and R₂₃ₐ may each independently be hydrogen, deuterium, —F, —Cl, —Br, —I, —SF₅, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₁-C₆₀ alkylthio group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₇-C₆₀ alkyl aryl group, a substituted or unsubstituted C₇-C₆₀ aryl alkyl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted C₂-C₆₀ alkyl heteroaryl group, a substituted or unsubstituted C₂-C₆₀ heteroaryl alkyl group, a substituted or unsubstituted C₁-C₆₀ heteroaryloxy group, a substituted or unsubstituted C₁-C₆₀ heteroarylthio group, a substituted or unsubstituted monovalent non-aromatic condensed polycyclic group, a substituted or unsubstituted monovalent non-aromatic condensed heteropolycyclic group, —N(Q₁)(Q₂), —Si(Q₃)(Q₄)(Q₅), —Ge(Q₃)(Q₄)(Q₅), —B(Q₆)(Q₇), —P(═O)(Q₈)(Q₉), or —P(Q₈)(Q₉).

At least one substituent of the substituted C₅-C₃₀ carbocyclic group, the substituted C₁-C₃₀ heterocyclic group, the substituted C₁-C₆₀ alkyl group, the substituted C₂-C₆₀ alkenyl group, the substituted C₂-C₆₀ alkynyl group, the substituted C₁-C₆₀ alkoxy group, the substituted C₁-C₆₀ alkylthio group, the substituted C₃-C₁₀ cycloalkyl group, the substituted C₁-C₁₀ heterocycloalkyl group, the substituted C₃-C₁₀ cycloalkenyl group, the substituted C₁-C₁₀ heterocycloalkenyl group, the substituted C₆-C₆₀ aryl group, the substituted C₇-C₆₀ alkyl aryl group, the substituted C₇-C₆₀ aryl alkyl group, the substituted C₆-C₆₀ aryloxy group, the substituted C₆-C₆₀ arylthio group, the substituted C₁-C₆₀ heteroaryl group, the substituted C₂-C₆₀ alkyl heteroaryl group, the substituted C₂-C₆₀ heteroaryl alkyl group, the substituted C₁-C₆₀ heteroaryloxy group, the substituted C₁-C₆₀ heteroarylthio group, the substituted monovalent non-aromatic condensed polycyclic group, and the substituted monovalent non-aromatic condensed heteropolycyclic group may be:
deuterium, —F, —Cl, —Br, —I, —SF₅, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, or a C₁-C₆₀ alkylthio group;
a C₁-C₆₀ alkyl group, a C₂-C₆₀ alkenyl group, a C₂-C₆₀ alkynyl group, a C₁-C₆₀ alkoxy group, or a C₁-C₆₀ alkylthio group, each substituted with at least one of deuterium, —F, —Cl, —Br, —I, —SF₅, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₇-C₆₀ alkyl aryl group, a C₆-C₆₀ aryloxy group, a C₆-C₆₀ arylthio group, a C₁-C₆₀ heteroaryl group, a C₂-C₆₀ alkyl heteroaryl group, a C₁-C₆₀ heteroaryloxy group, a C₁-C₆₀ heteroarylthio group, a monovalent non-aromatic condensed polycyclic group, a monovalent non-aromatic condensed heteropolycyclic group, —N(Q₁₁)(Q₁₂), —Si(Q₁₃)(Q₁₄)(Q₁₅), —Ge(Q₁₃)(Q₁₄)(Q₁₅), —B(Q₁₆)(Q₁₇), —P(═O)(Q₁₈)(Q₁₉), —P(Q₁₈)(Q₁₉), or a combination thereof;
a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₇-C₆₀ alkyl aryl group, a C₆-C₆₀ aryloxy group, a C₆-C₆₀ arylthio group, a C₁-C₆₀ heteroaryl group, a C₂-C₆₀ alkyl heteroaryl group, a C₁-C₆₀ heteroaryloxy group, a C₁-C₆₀ heteroarylthio group, a monovalent non-aromatic condensed polycyclic group, or a monovalent non-aromatic condensed heteropolycyclic group;
a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₇-C₆₀ alkyl aryl group, a C₆-C₆₀ aryloxy group, a C₆-C₆₀ arylthio group, a C₁-C₆₀ heteroaryl group, a C₂-C₆₀ alkyl heteroaryl group, a C₁-C₆₀ heteroaryloxy group, a C₁-C₆₀ heteroarylthio group, a monovalent non-aromatic condensed polycyclic group, or a monovalent non-aromatic condensed heteropolycyclic group, each substituted with at least one of deuterium, —F, —Cl, —Br, —I, —SF₅, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₁-C₆₀ alkylthio group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₇-C₆₀ alkyl aryl group, a C₇-C₆₀ aryl alkyl group, a C₆-C₆₀ aryloxy group, a C₆-C₆₀ arylthio group, a C₁-C₆₀ heteroaryl group, a C₂-C₆₀ alkyl heteroaryl group, a C₂-C₆₀ heteroaryl alkyl group, a C₁-C₆₀ heteroaryloxy group, a C₁-C₆₀ heteroarylthio group, a monovalent non-aromatic condensed polycyclic group, a monovalent non-aromatic condensed heteropolycyclic group, —N(Q₂₁)(Q₂₂), —Si(Q₂₃)(Q₂₄)(Q₂₅), —Ge(Q₂₃)(Q₂₄)(Q₂₅), —B(Q₂₆)(Q₂₇), —P(═O)(Q₂₈)(Q₂₉), —P(Q₂₈)(Q₂₉), or a combination thereof; or
—N(Q₃₁)(Q₃₂), —Si(Q₃₃)(Q₃₄)(Q₃₅), —Ge(Q₃₃)(Q₃₄)(Q₃₅), —B(Q₃₆)(Q₃₇), —P(═O)(Q₃₈)(Q₃₉), or —P(Q₃₈)(Q₃₉), and
Q₁ to Q₉, Q₁₁ to Q₁₉, Q₂₁ to Q₂₉, and Q₃₁ to Q₃₉ may each independently be hydrogen, deuterium, —F, —Cl, —Br, —I, —SF₅, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₁-C₆₀ alkylthio group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₇-C₆₀ alkyl aryl group, a substituted or unsubstituted C₇-C₆₀ aryl alkyl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted C₂-C₆₀ alkyl heteroaryl group, a substituted or unsubstituted C₂-C₆₀ heteroaryl alkyl group, a substituted or unsubstituted C₁-C₆₀ heteroaryloxy group, a substituted or unsubstituted C₁-C₆₀ heteroarylthio group, a substituted or unsubstituted monovalent non-aromatic condensed polycyclic group, or a substituted or unsubstituted monovalent non-aromatic condensed heteropolycyclic group.

In Formula E-2, b21 to b24 each indicate the number of R₂₁(s) to R₂₄(s), respectively, and may each independently be an integer from 0 to 10 (for example, 0, 1, 2, or 3). When b21 is 2 or greater, two or more of R₂₁ may be identical to or different from each other, when b22 is 2 or greater, two or more of R₂₂ may be identical to or different from each other, when b23 is 2 or greater, two or more of R₂₃ may be identical to or different from each other, and when b24 is 2 or greater, two or more of R₂₄ may be identical to or different from each other.

For example, in Formula E-2, R₂₁ to R₂₄, R₂₁ₐ, R₂₂ₐ, and R₂₃ₐ may each independently be:
hydrogen, deuterium, —F, —Cl, —Br, —I, —SF₅, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, —SF₅, a C₁-C₂₀ alkyl group, a C₁-C₂₀ alkoxy group, or a C₁-C₂₀ alkylthio group;
a C₁-C₂₀ alkyl group, a C₁-C₂₀ alkoxy group, or a C₁-C₂₀ alkylthio group, each substituted with at least one of deuterium, —F, —Cl, —Br, —I, —SF₅, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a naphthyl group, a pyridinyl group, a pyrimidinyl group, or a combination thereof;
a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, or an imidazopyrimidinyl group;
a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, or an imidazopyrimidinyl group, each substituted with at least one of deuterium, —F, —Cl, —Br, —I, —SF₅, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, a C₁-C₂₀ alkylthio group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a phenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, an isobenzothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, an imidazopyrimidinyl group, —Si(Q₃₃)(Q₃₄)(Q₃₅), or a combination thereof; or
—N(Q₁)(Q₂), —Si(Q₃)(Q₄)(Q₅), —Ge(Q₃)(Q₄)(Q₅), —B(Q₆)(Q₇), —P(═O)(Q₈)(Q₉), or —P(Q₈)(Q₉), and
Q₁ to Q₉ and Q₃₁ to Q₃₃ may each independently be:
—CH₃, —CD₃, —CD₂H, —CDH₂, —CH₂CH₃, —CH₂CD₃, —CH₂CD₂H, —CH₂CDH₂, —CHDCH₃, —CHDCD₂H, —CHDCDH₂, —CHDCD₃, —CD₂CD₃, —CD₂CD₂H, or —CD₂CDH₂;
an n-propyl group, an isopropyl group, an n-butyl group, an isobutyl group, a sec-butyl group, a tert-butyl group, an n-pentyl group, an isopentyl group, a sec-pentyl group, a tert-pentyl group, a phenyl group, or a naphthyl group; or
an n-propyl group, an isopropyl group, an n-butyl group, an isobutyl group, a sec-butyl group, a tert-butyl group, an n-pentyl group, an isopentyl group, a sec-pentyl group, a tert-pentyl group, a phenyl group, or a naphthyl group, each substituted with at least one of deuterium, a C₁-C₁₀ alkyl group, a phenyl group, or a combination thereof.

In one or more embodiments, R₂₁ to R₂₄, R₂₁ₐ, R₂₂ₐ, and R₂₃ₐ may each independently be:
hydrogen, deuterium, —F, —Cl, —Br, —I, —SF₅, —CD₃, —CD₂H, —CDH₂, —CF₃, —CF₂H, —CFH₂, a C₁-C₆₀ alkyl group, a C₂-C₆₀ alkenyl group, a C₂-C₆₀ alkynyl group, a C₁-C₆₀ alkoxy group, or a C₁-C₆₀ alkylthio group; or
a group represented by one of Formulae 9-1 to 9-61, 9-201 to 9-240, 10-1 to 10-129, or 10-201 to 10-355; or
—N(Q₁)(Q₂), —Si(Q₃)(Q₄)(Q₅) or —Ge(Q₃)(Q₄)(Q₅), and
Q₁ to Q₅ are each as described herein:

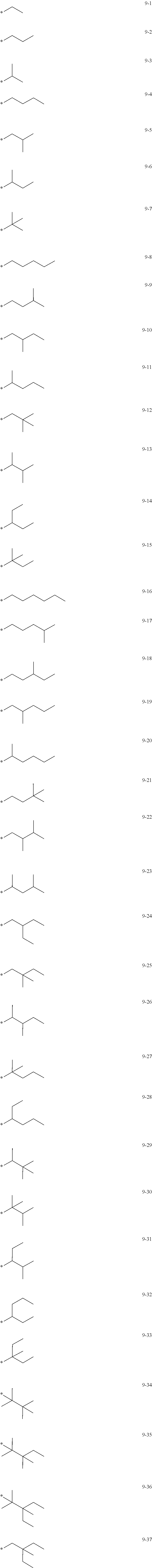

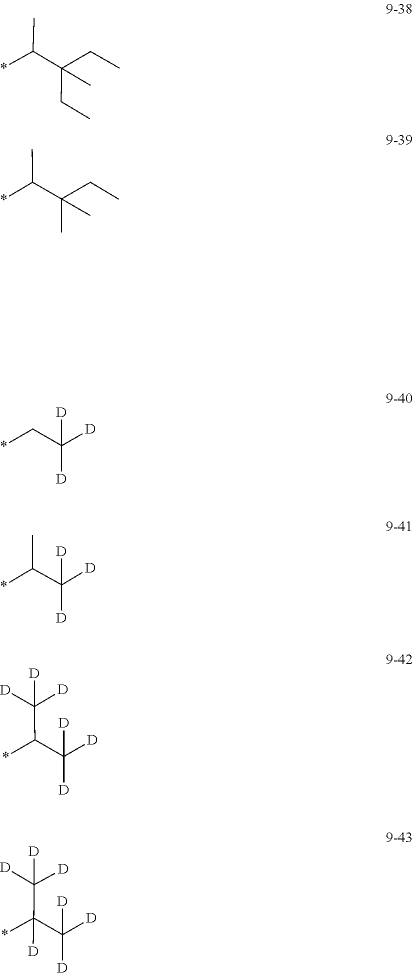

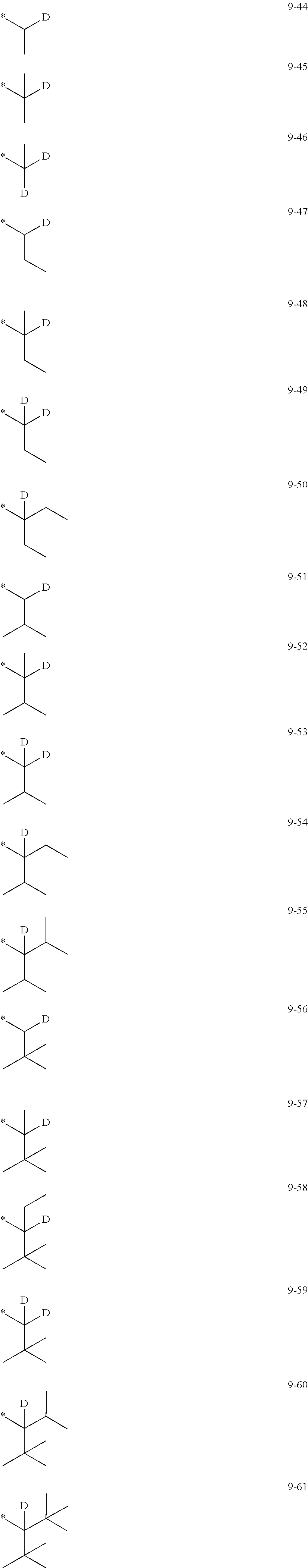

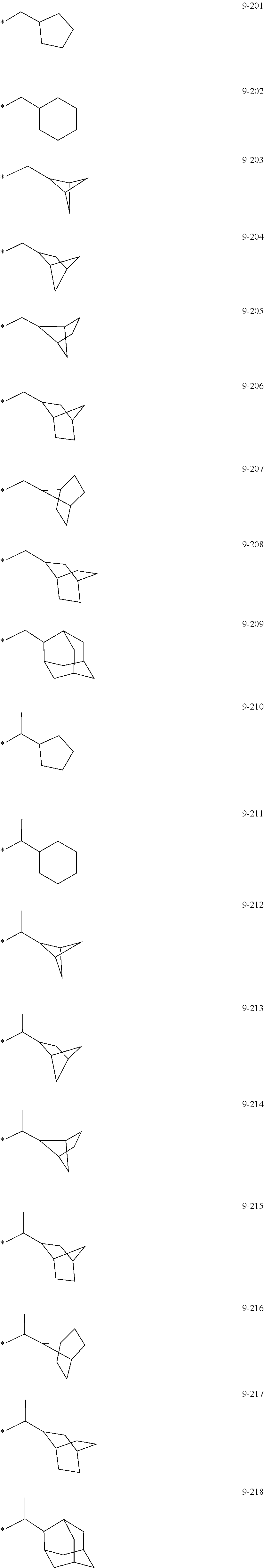

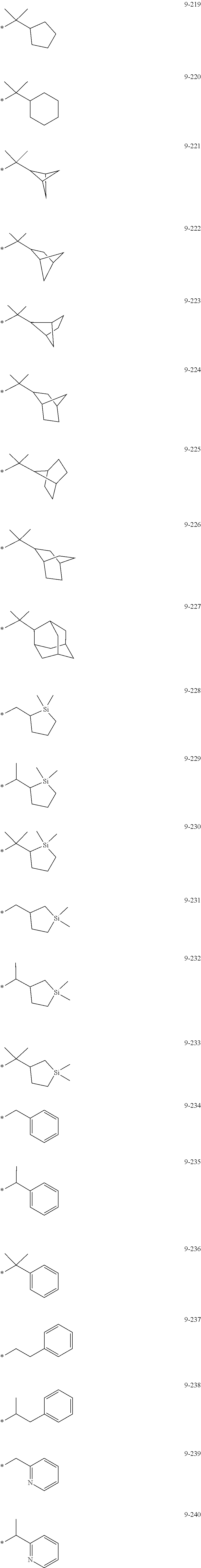

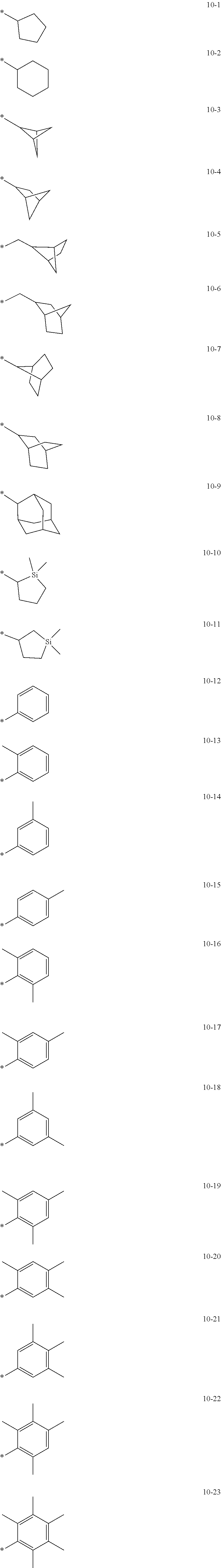

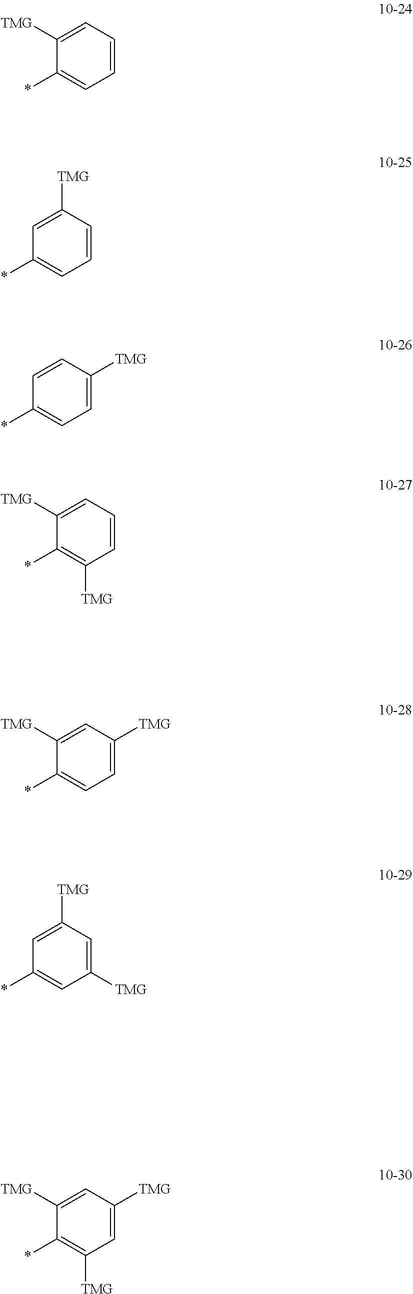

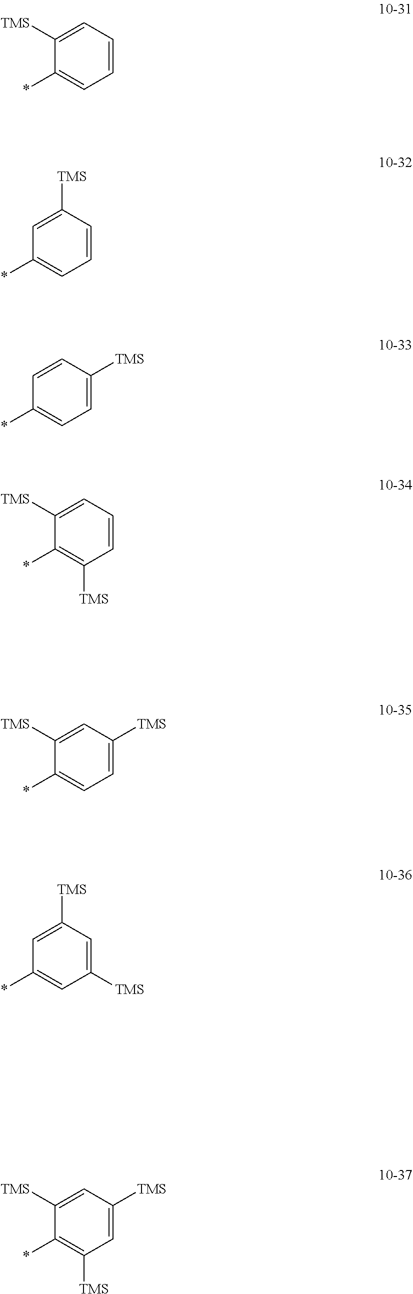

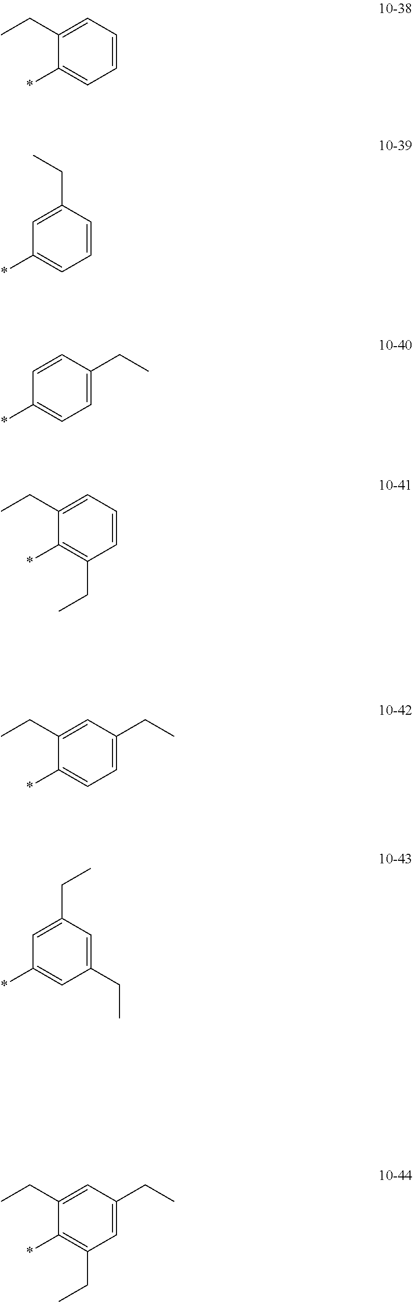

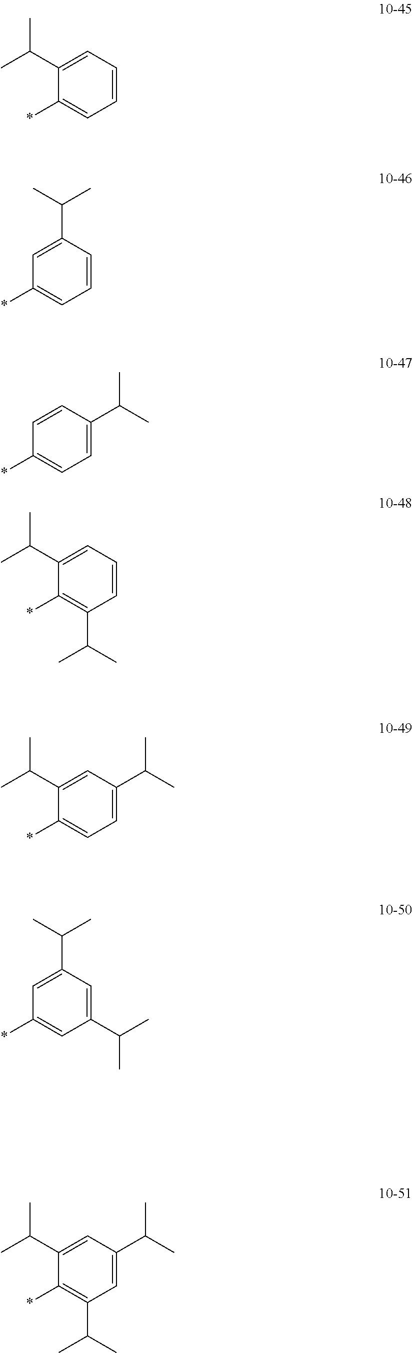

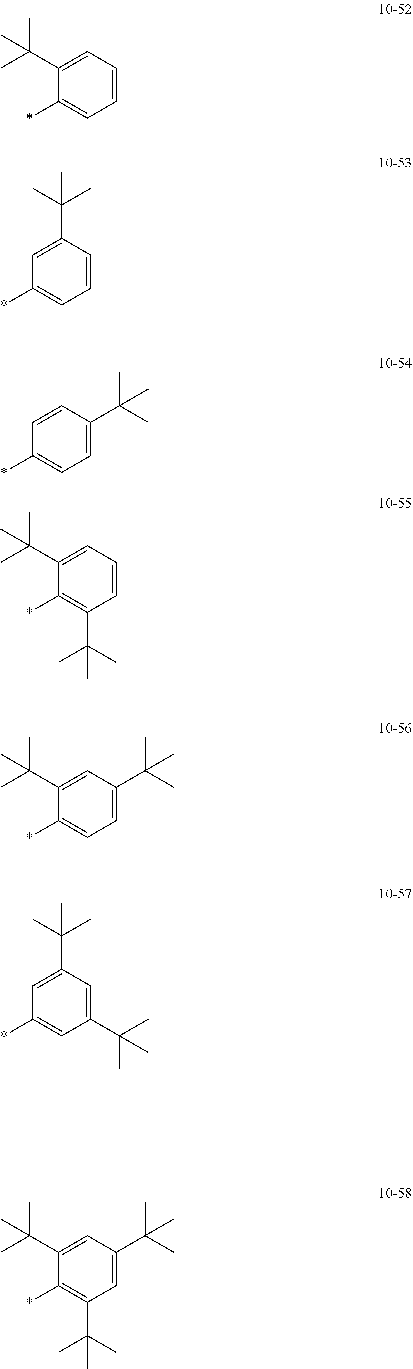

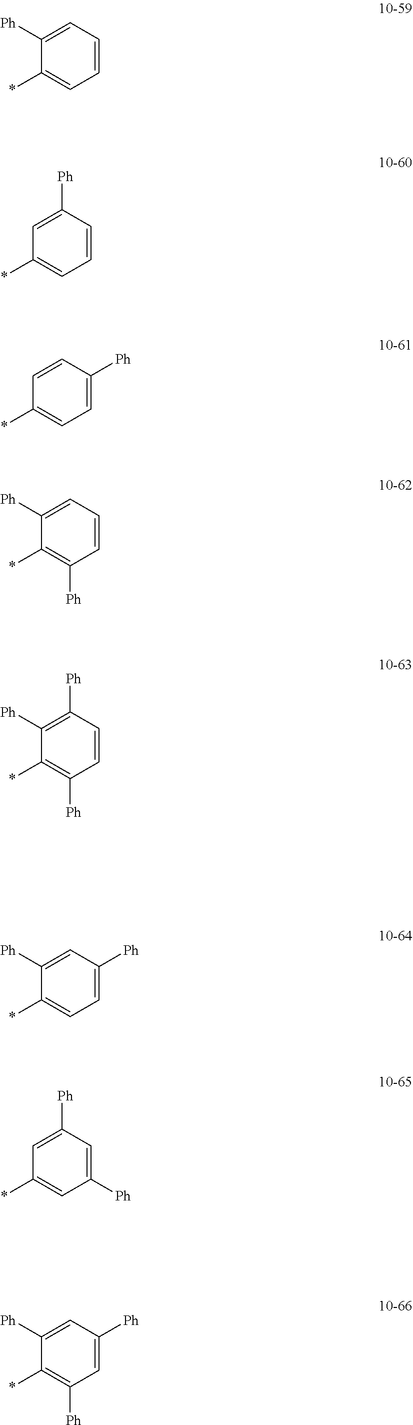

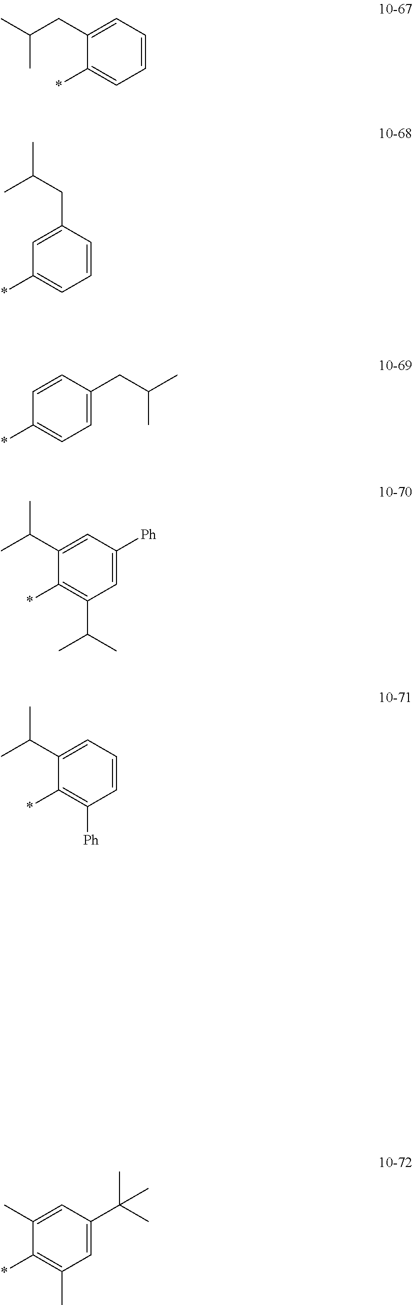

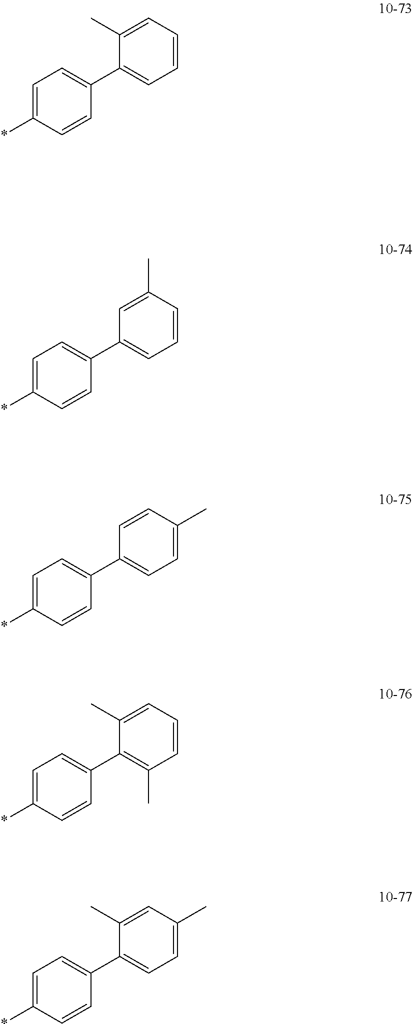

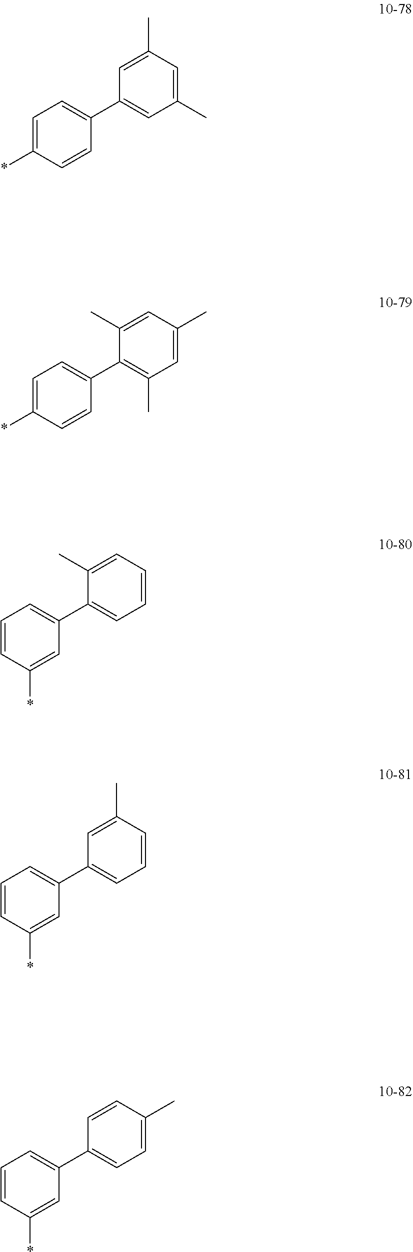

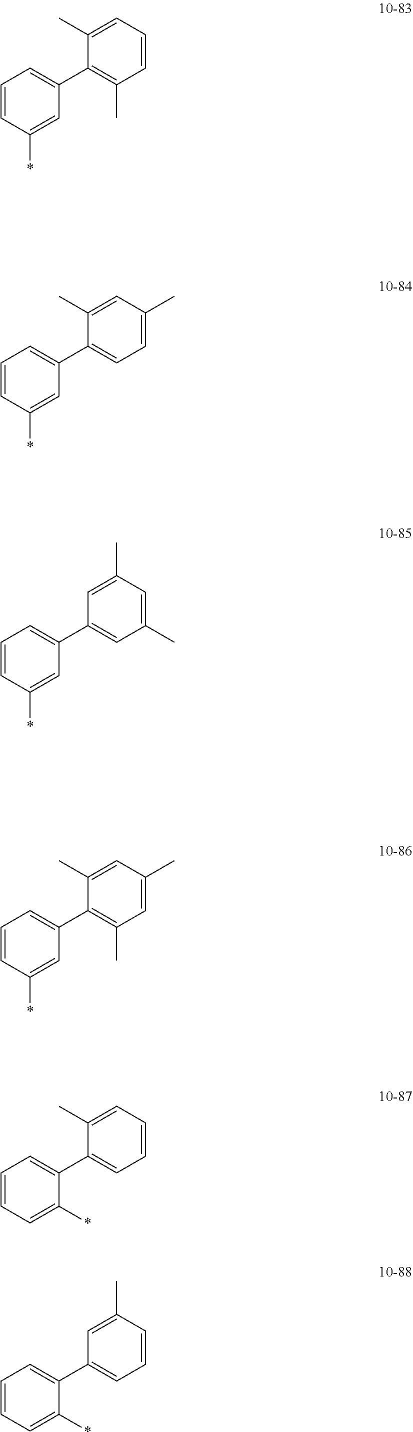

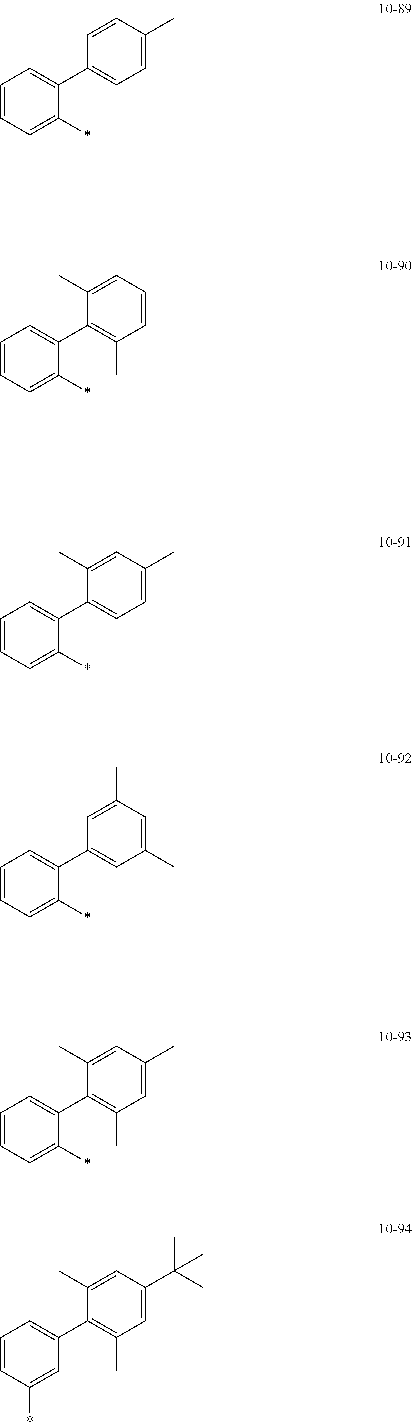

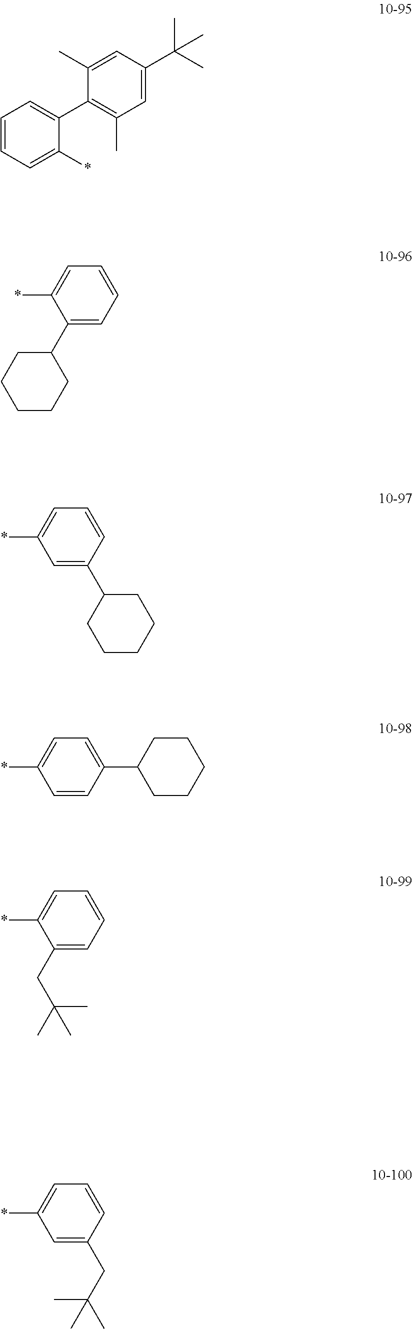

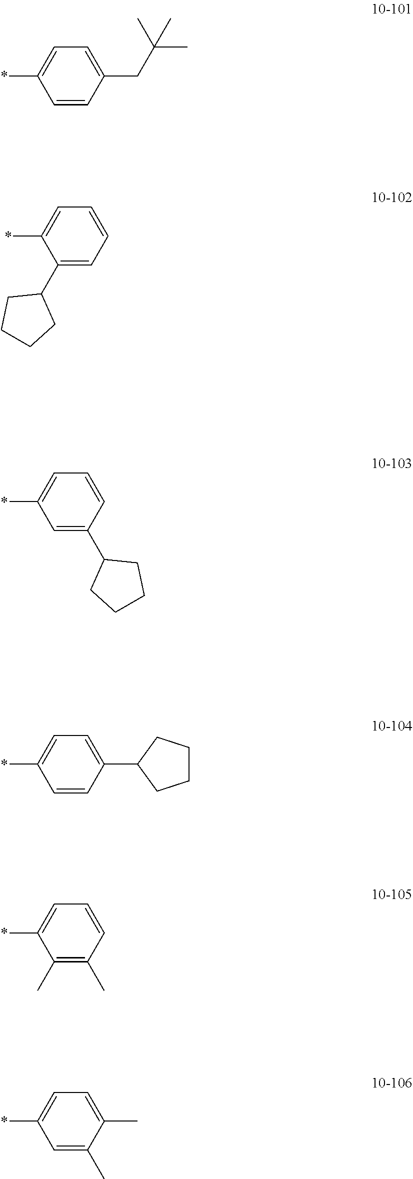

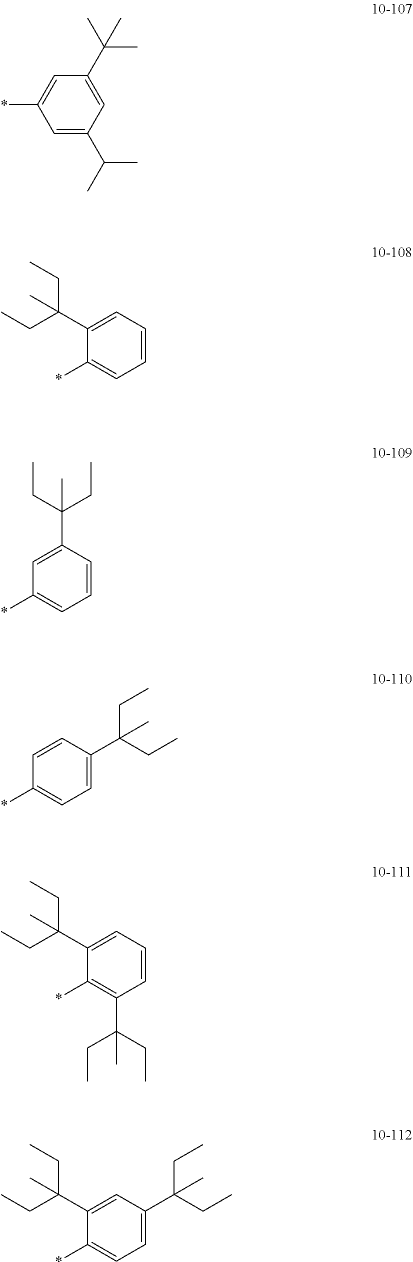

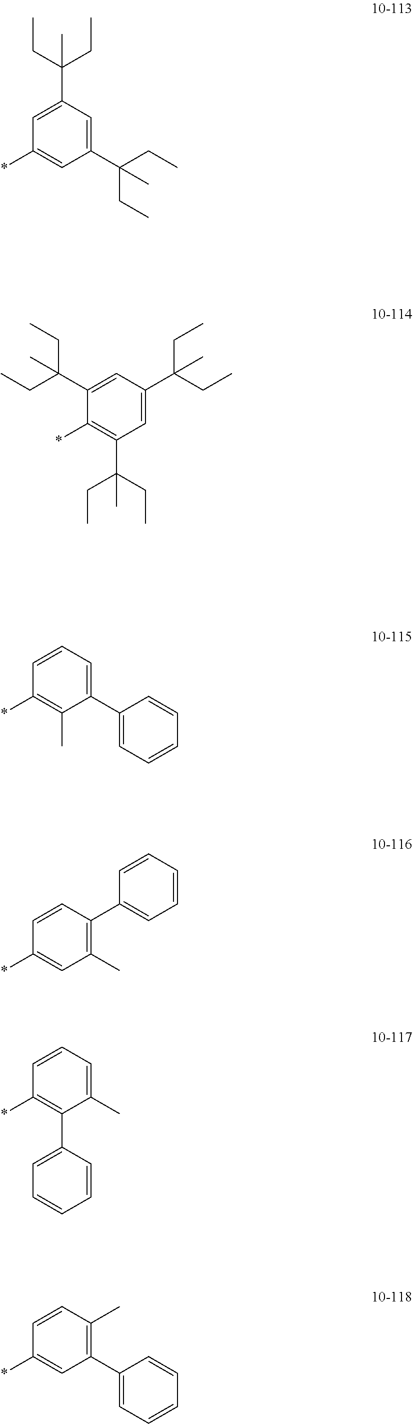

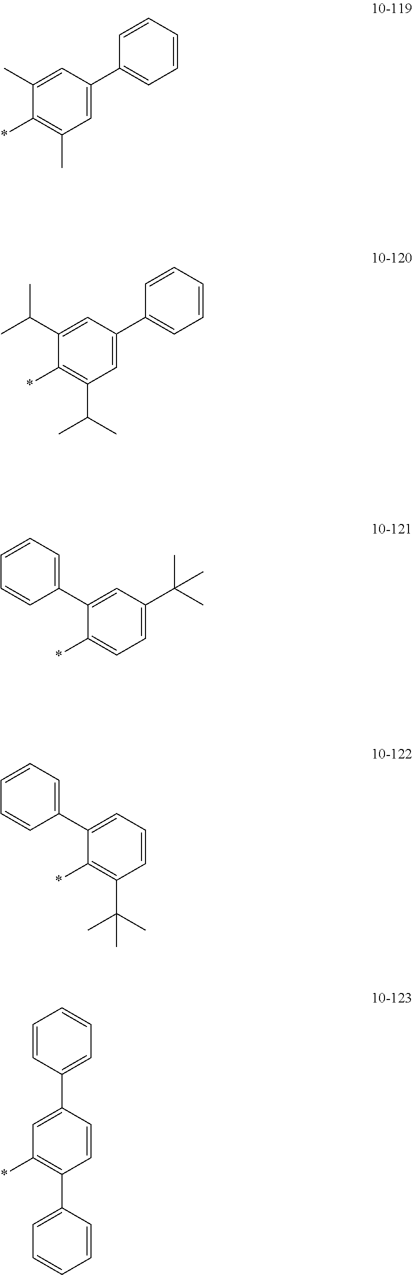

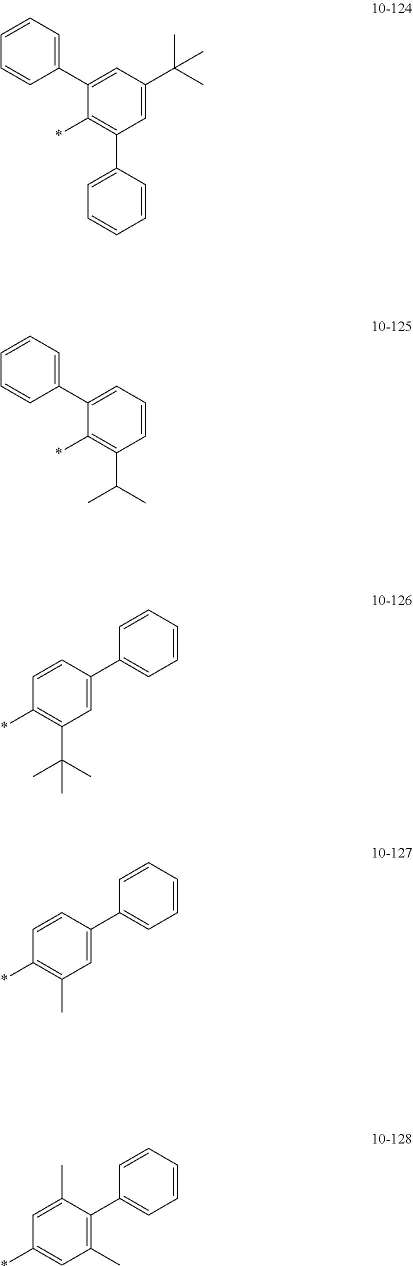

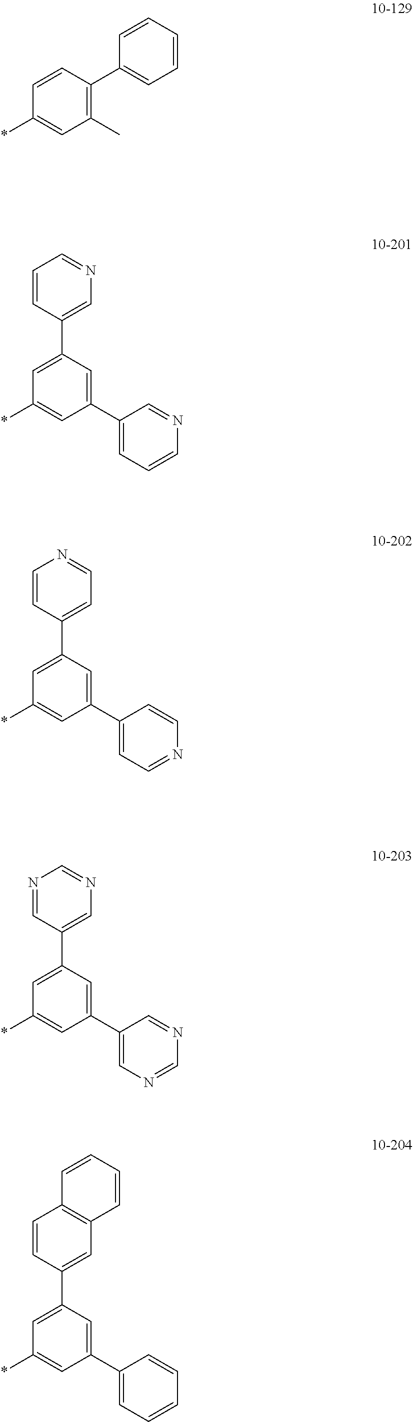

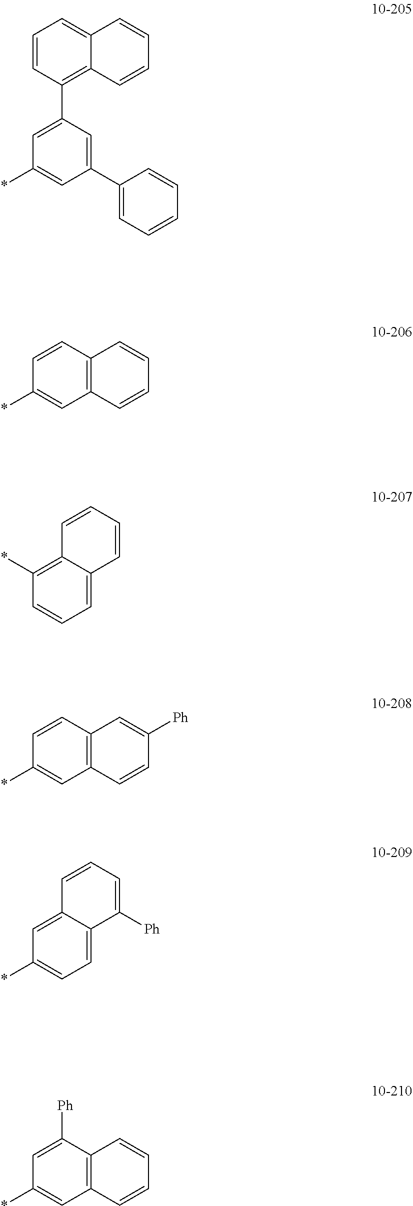

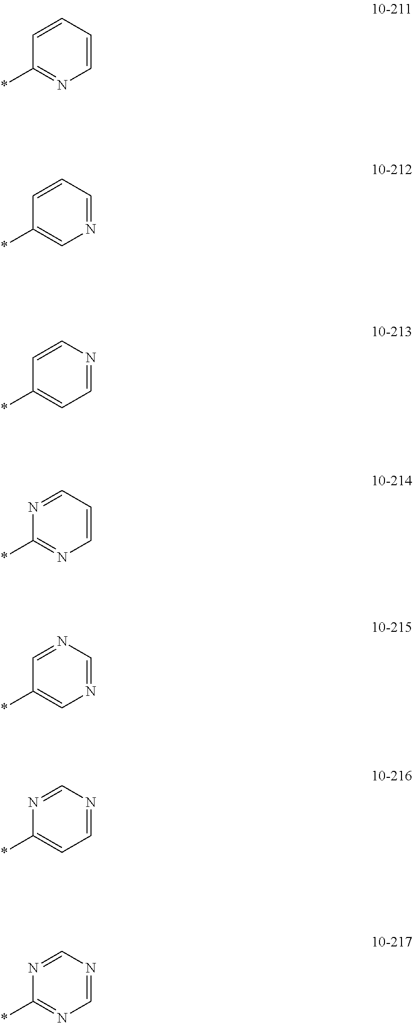

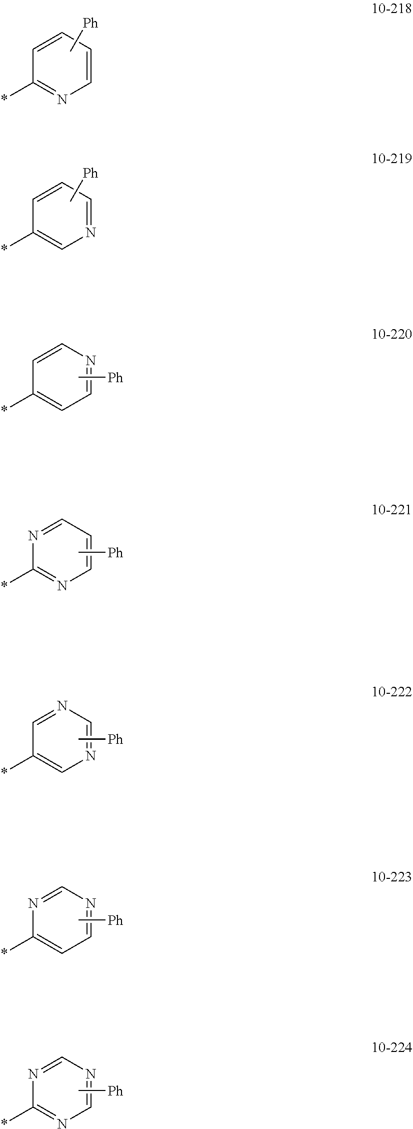

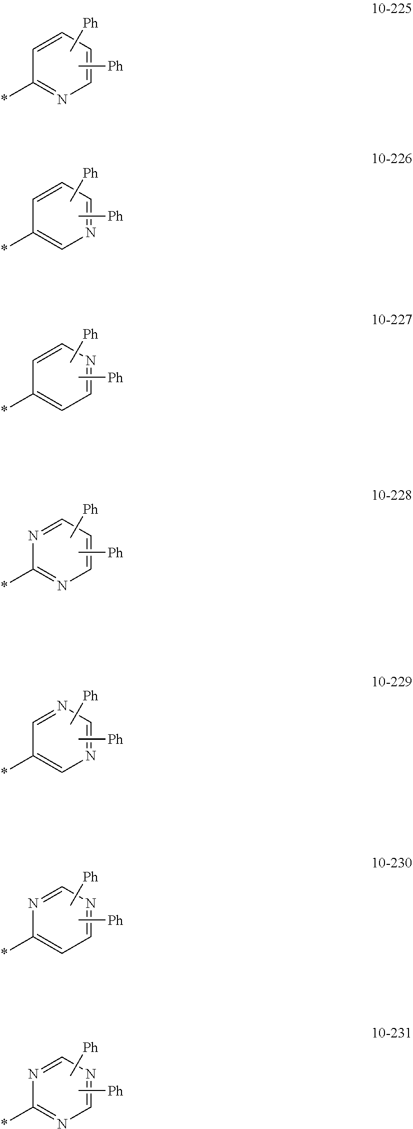

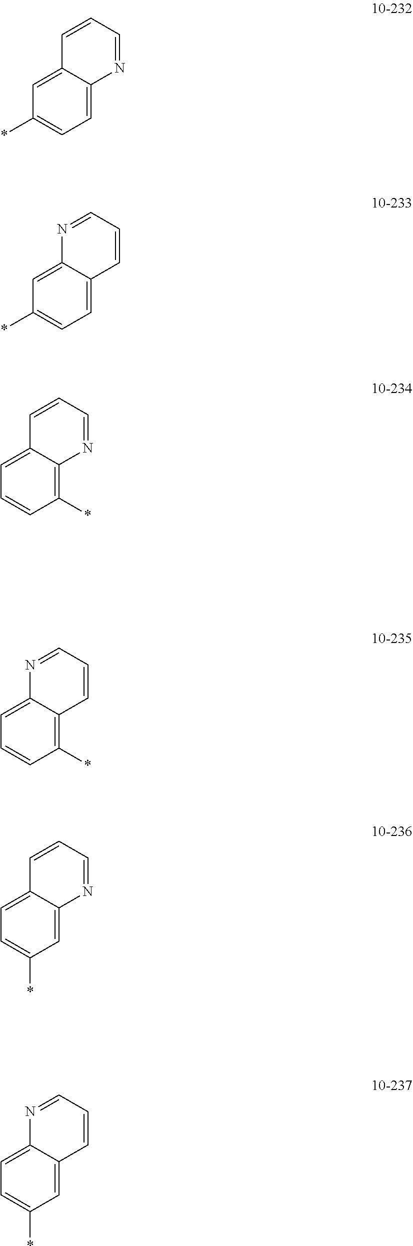

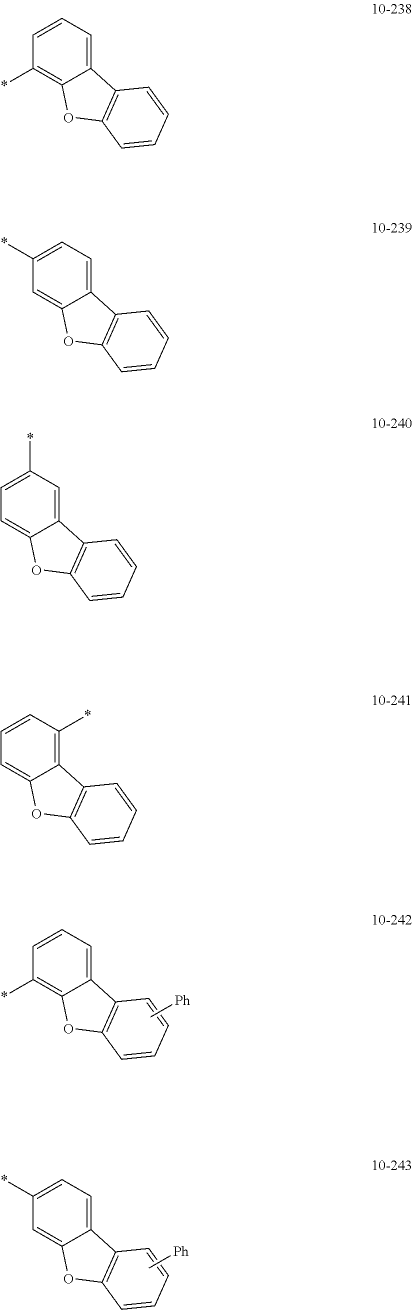

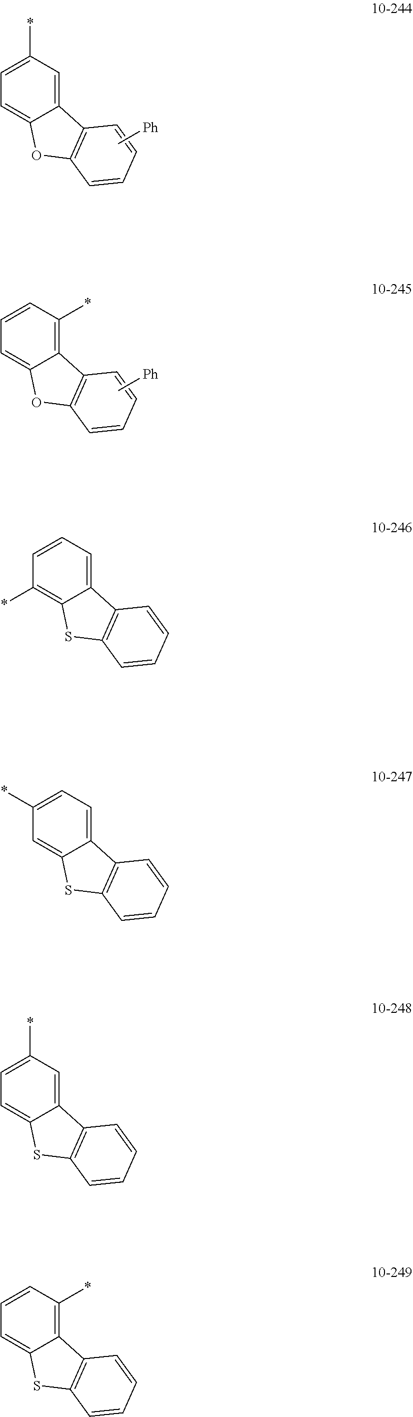

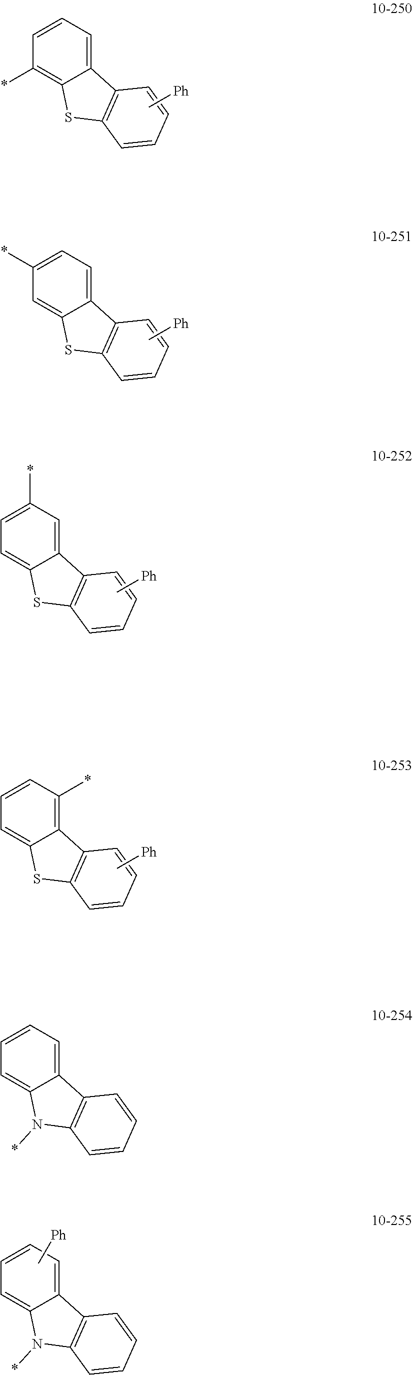

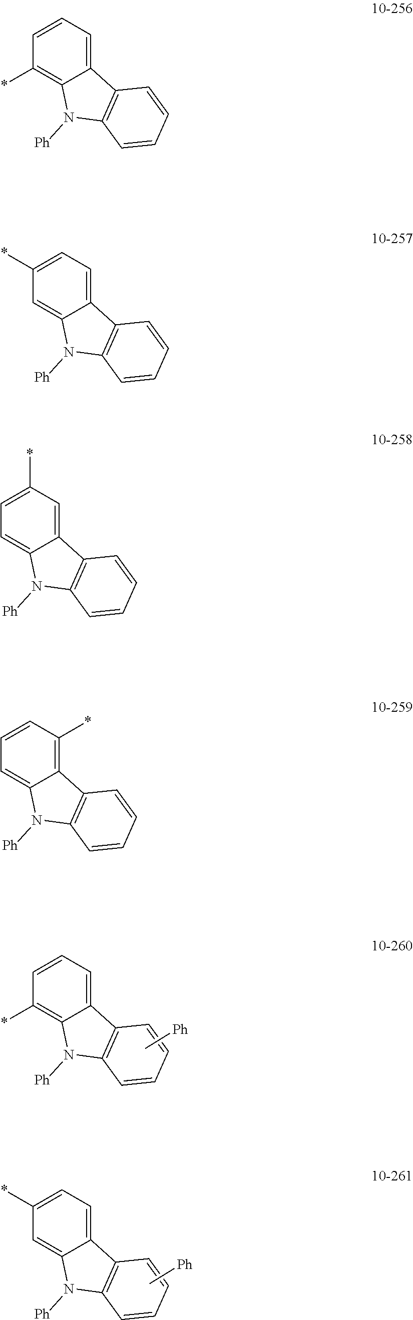

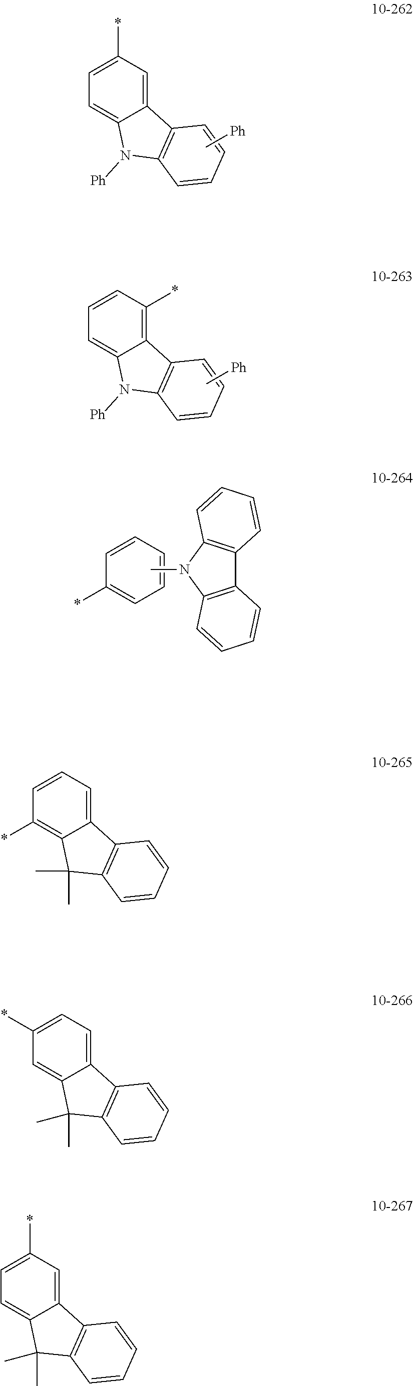

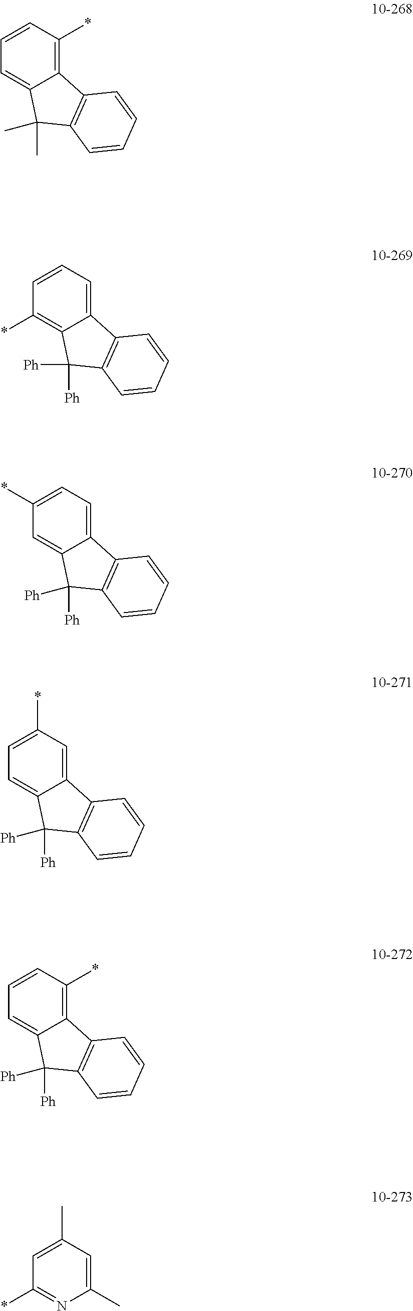

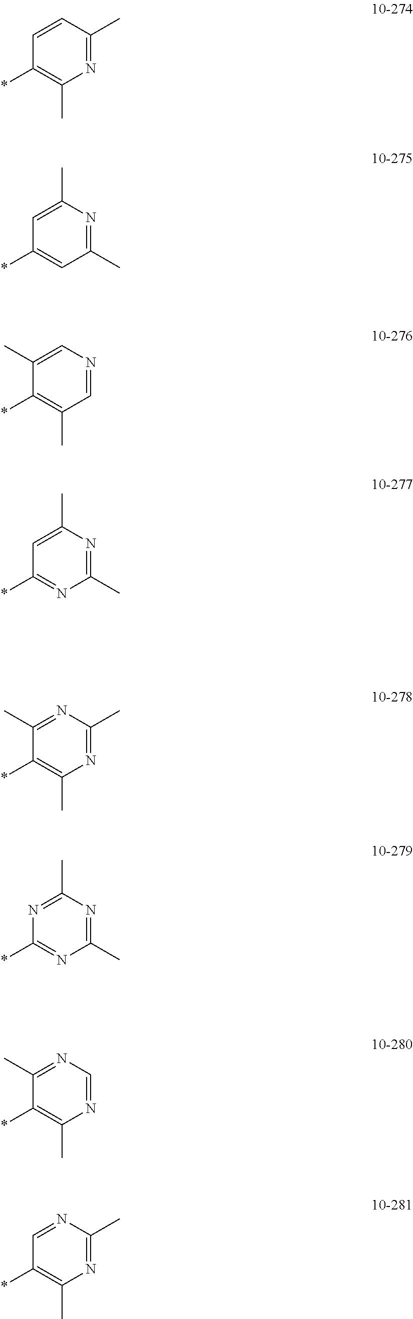

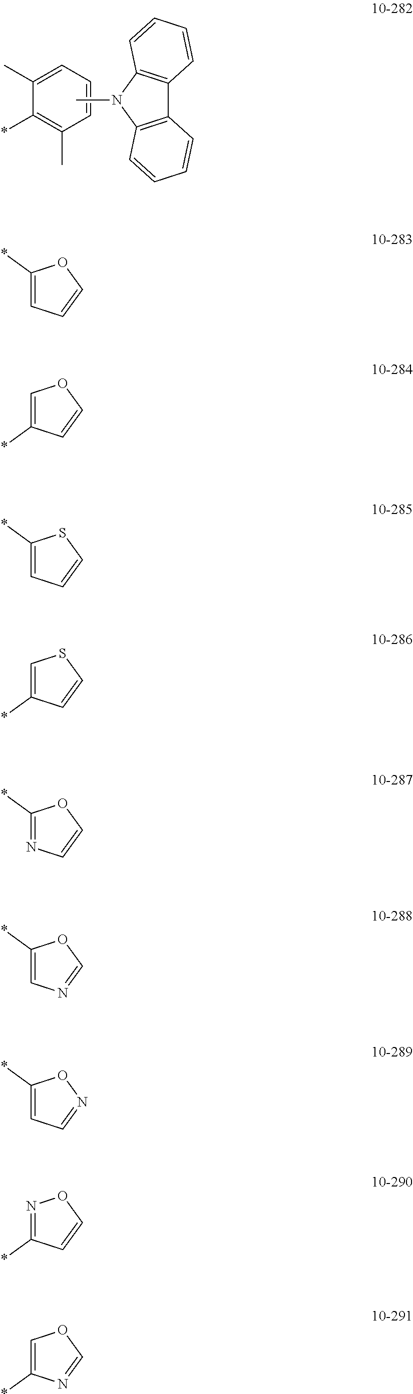

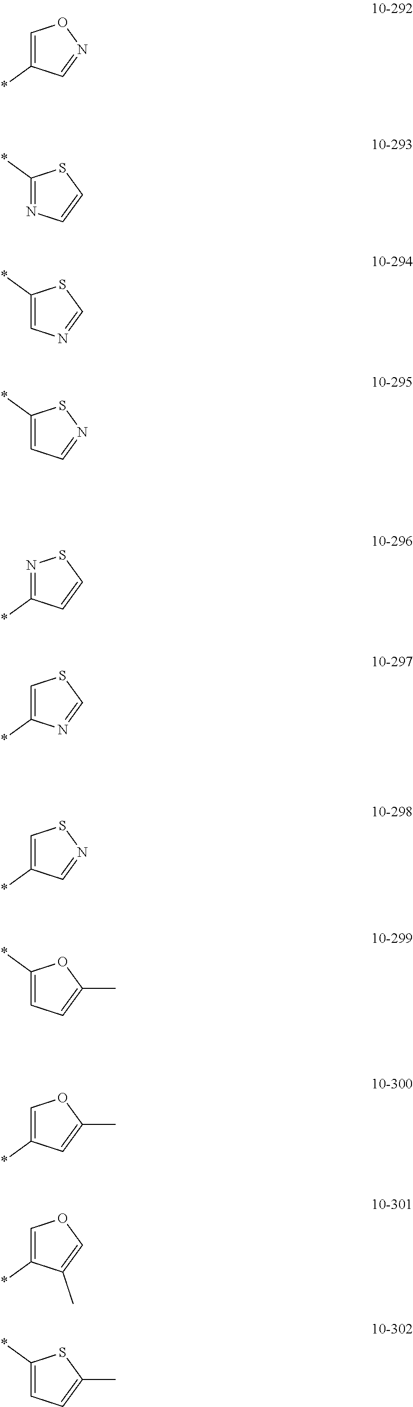

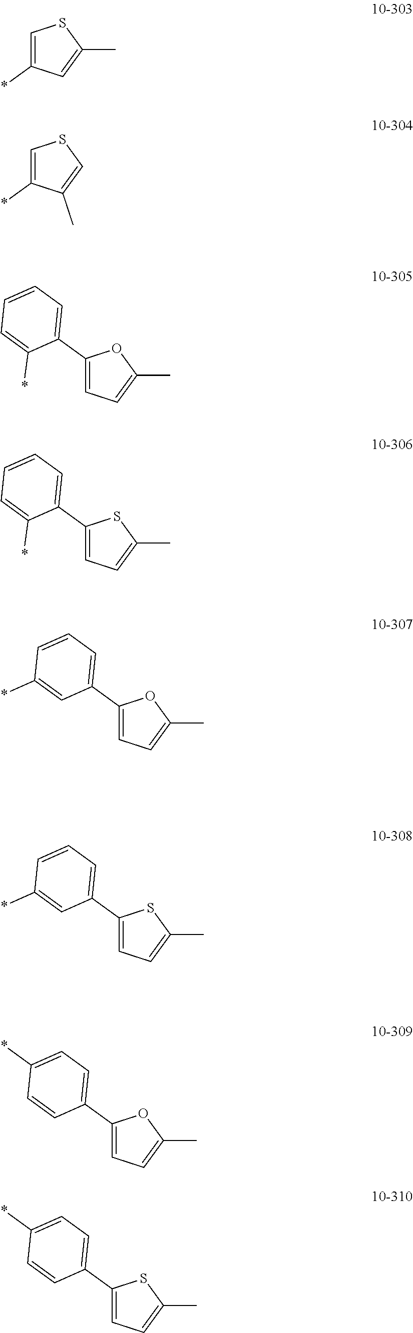

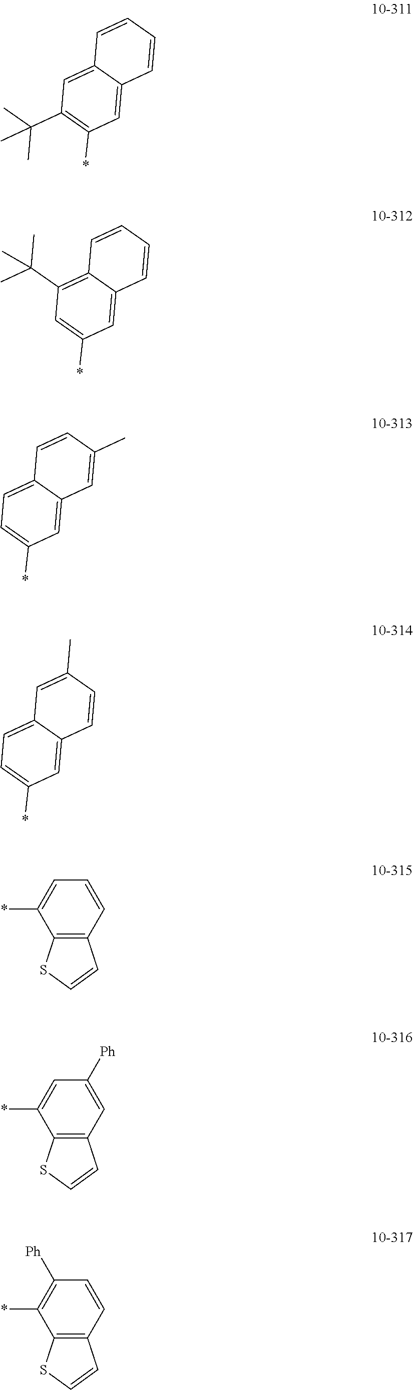

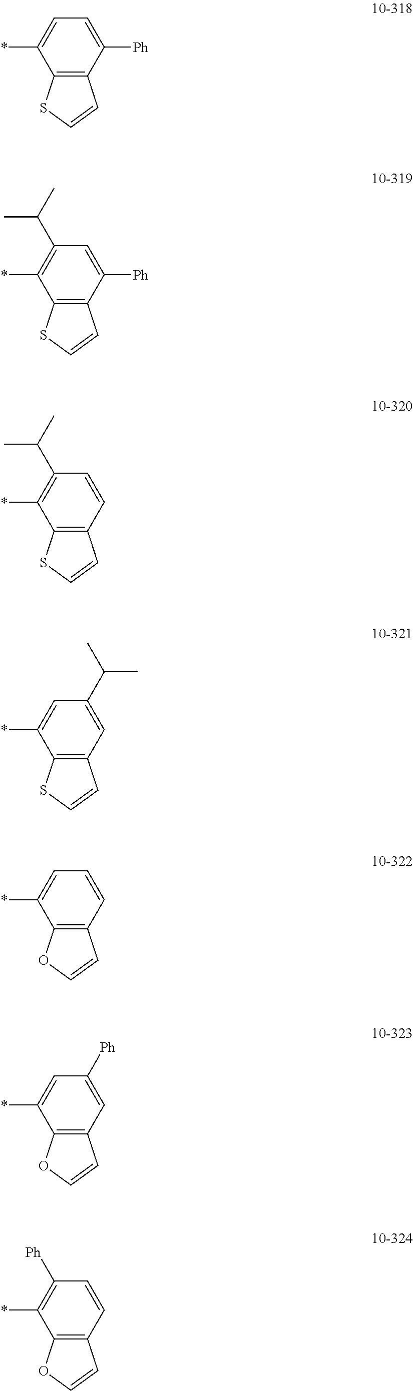

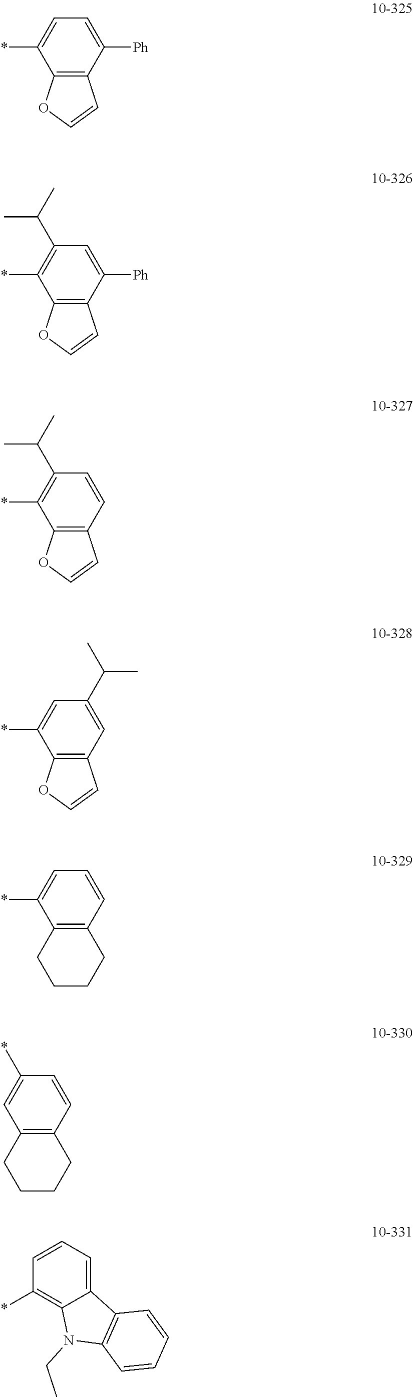

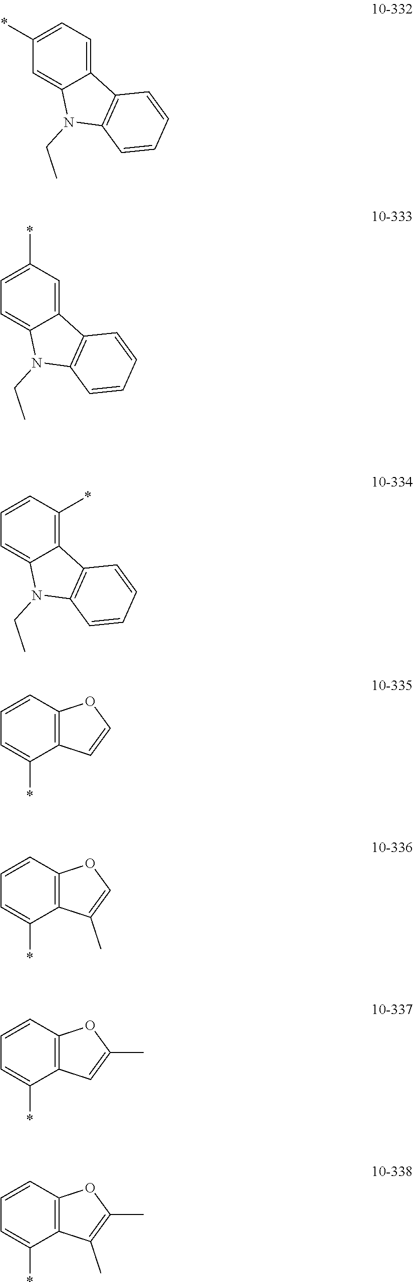

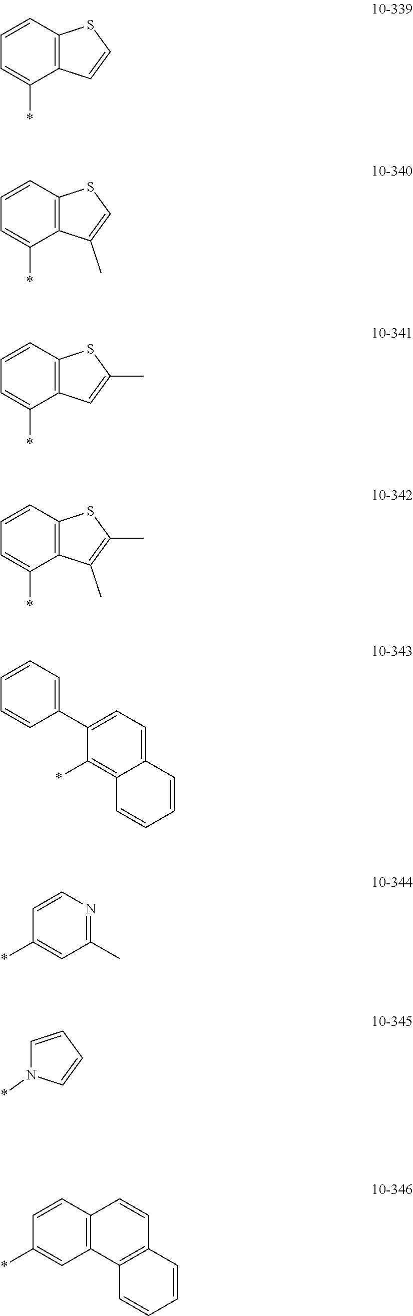

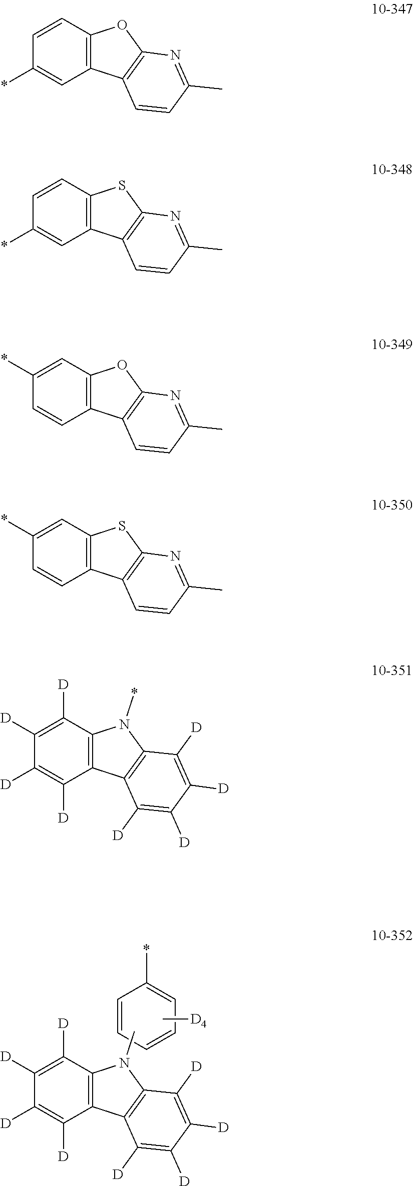

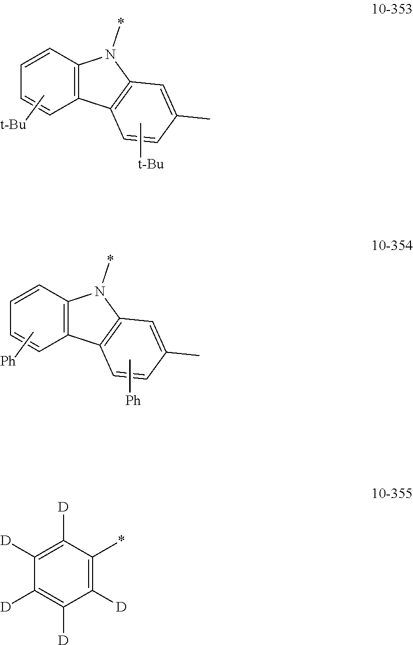

wherein, in Formulae 9-1 to 9-61, 9-201 to 9-240, 10-1 to 10-129, and 10-201 to 10-355, * indicates a binding site to a neighboring atom, “Ph” represents a phenyl group, “TMS” represents a trimethylsilyl group, “TMG” represents a trimethylgermyl group, and “t-Bu” represents a t-butyl group.

In one or more embodiments, R₂₁ and R₂₂ may each independently be:
a phenyl group, a naphthyl group, or a carbazolyl group, each unsubstituted or substituted with at least one of deuterium, a C₁-C₁₀ alkyl group, a phenyl group, —Si(Q₃₃)(Q₃₄)(Q₃₅), or a combination thereof; or
—Si(Q₃)(Q₄)(Q₅),
Q₃ to Q₅ may each independently be a phenyl group or a naphthyl group, each substituted with at least one of deuterium, a C₁-C₁₀ alkyl group, a phenyl group, or —Si(Q₃₃)(Q₃₄)(Q₃₅), and
Q₃₃ to Q₃₅ may each independently be a phenyl group or a naphthyl group, each substituted with at least one of deuterium, a C₁-C₁₀ alkyl group, or a phenyl group.

In one or more embodiments, R₂₁ and R₂₂ may each independently be a C₆-C₆₀ aryl group unsubstituted or substituted with at least one R₁₀ₐ, a group represented by Formula E-2A, or a group represented by Formula E-2B, and
R₁₀ₐ may be as described in connection with R₂₃ in Formula 2-E:

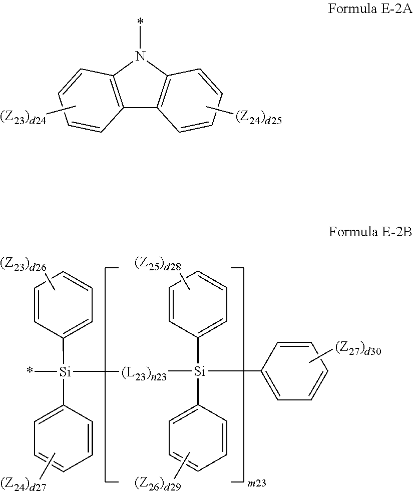

wherein, in Formulae E-2A and E-2B,
Z₂₃ to Z₂₇ are each as described in connection with R₂₃,
d24 and d25 may each independently be an integer from 1 to 4,
d26 to d30 may each independently be an integer from 1 to 5,
L₂₃ is as described in connection with L₂1,
n23 may be an integer from 1 to 5,
m23 may be an integer from 0 to 3, and
* indicates a binding site to a neighboring atom.

Non-limiting examples of the electron-transporting host include, for example, compounds of Groups HE1 to HE8, but embodiments are not limited thereto:

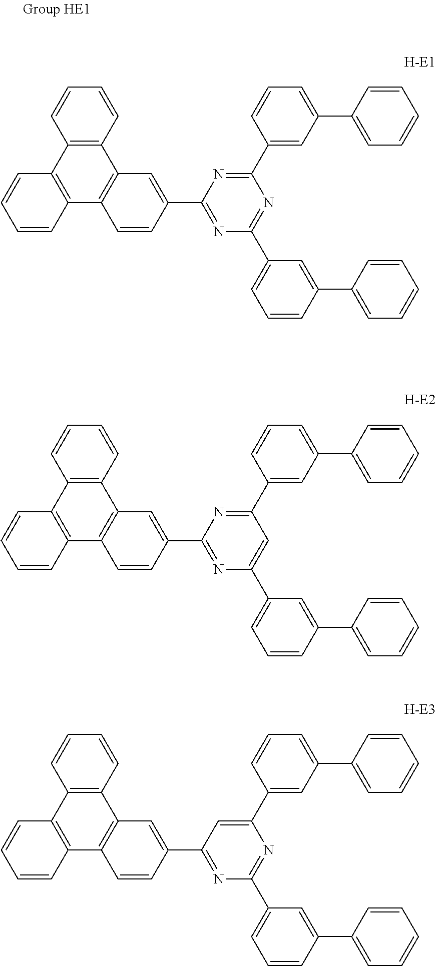 [SMILES: c1ccc(-c2cccc(-c3nc(-c4cccc(-c5ccccc5)c4)nc(-c4ccc5c6ccccc6c6ccccc6c5c4)n3)c2)cc1 ; c1ccc(-c2cccc(-c3cc(-c4cccc(-c5ccccc5)c4)nc(-c4ccc5c6ccccc6c6ccccc6c5c4)n3)c2)cc1 ; c1ccc(-c2cccc(-c3cc(-c4ccc5c6ccccc6c6ccccc6c5c4)nc(-c4cccc(-c5ccccc5)c4)n3)c2)cc1]

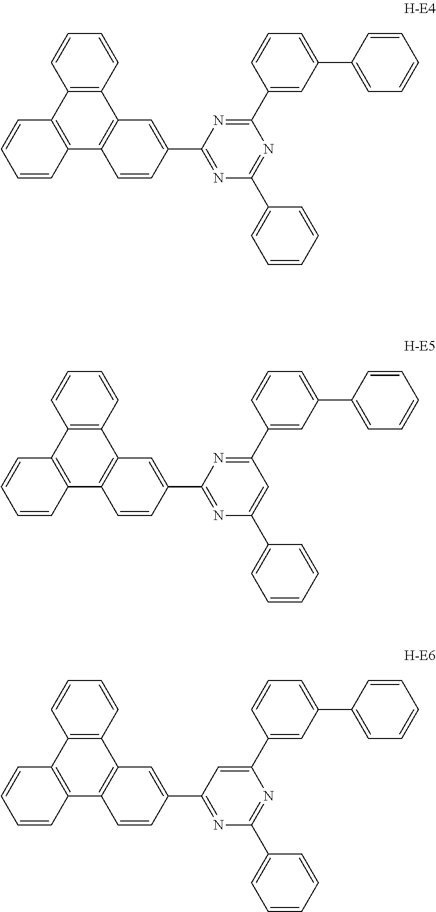 [SMILES: c1ccc(-c2cccc(-c3nc(-c4ccccc4)nc(-c4ccc5c6ccccc6c6ccccc6c5c4)n3)c2)cc1 ; c1ccc(-c2cccc(-c3cc(-c4ccccc4)nc(-c4ccc5c6ccccc6c6ccccc6c5c4)n3)c2)cc1 ; c1ccc(-c2cccc(-c3cc(-c4ccc5c6ccccc6c6ccccc6c5c4)nc(-c4ccccc4)n3)c2)cc1]

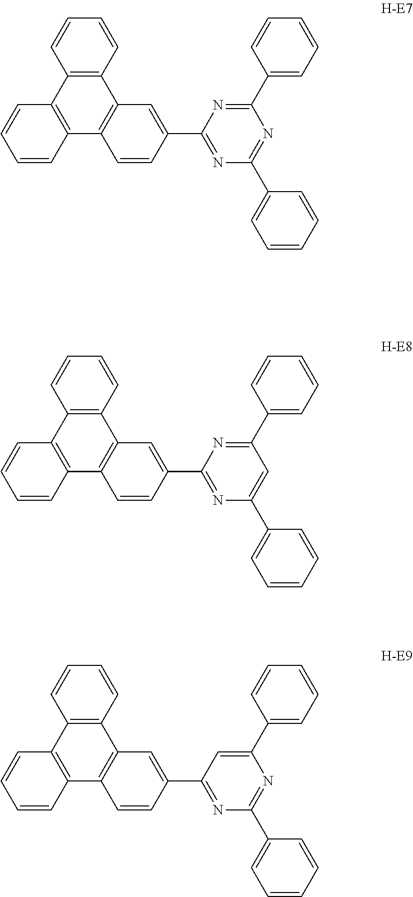 [SMILES: c1ccc(-c2nc(-c3ccccc3)nc(-c3ccc4c5ccccc5c5ccccc5c4c3)n2)cc1 ; c1ccc(-c2cc(-c3ccccc3)nc(-c3ccc4c5ccccc5c5ccccc5c4c3)n2)cc1 ; c1ccc(-c2cc(-c3ccc4c5ccccc5c5ccccc5c4c3)nc(-c3ccccc3)n2)cc1]

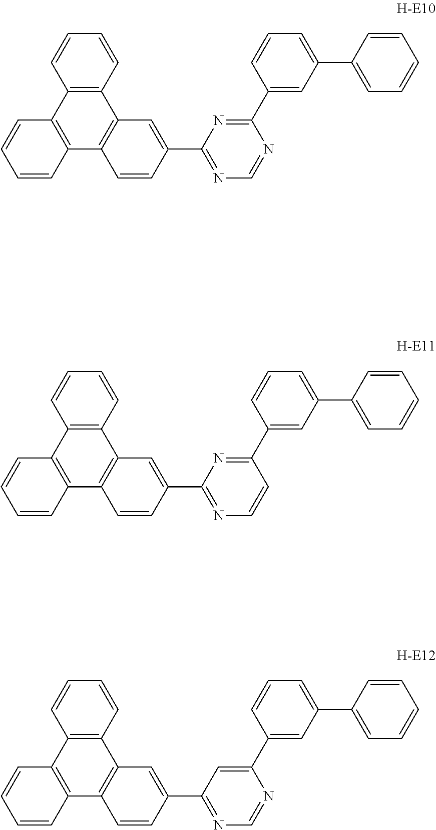 [SMILES: c1ccc(-c2cccc(-c3ncnc(-c4ccc5c6ccccc6c6ccccc6c5c4)n3)c2)cc1 ; c1ccc(-c2cccc(-c3ccnc(-c4ccc5c6ccccc6c6ccccc6c5c4)n3)c2)cc1 ; c1ccc(-c2cccc(-c3cc(-c4ccc5c6ccccc6c6ccccc6c5c4)ncn3)c2)cc1]

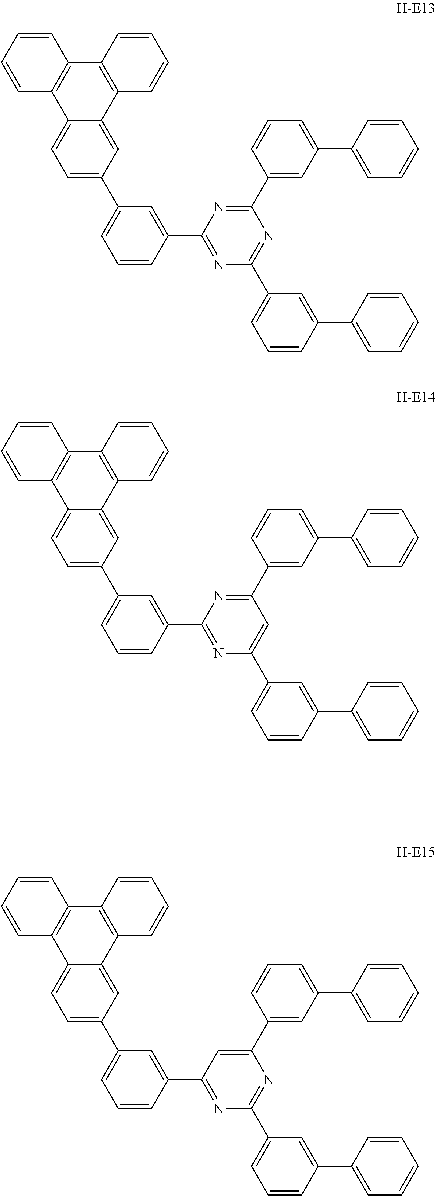 [SMILES: c1ccc(-c2cccc(-c3nc(-c4cccc(-c5ccccc5)c4)nc(-c4cccc(-c5ccc6c7ccccc7c7ccccc7c6c5)c4)n3)c2)cc1 ; c1ccc(-c2cccc(-c3cc(-c4cccc(-c5ccccc5)c4)nc(-c4cccc(-c5ccc6c7ccccc7c7ccccc7c6c5)c4)n3)c2)cc1 ; c1ccc(-c2cccc(-c3cc(-c4cccc(-c5ccc6c7ccccc7c7ccccc7c6c5)c4)nc(-c4cccc(-c5ccccc5)c4)n3)c2)cc1]

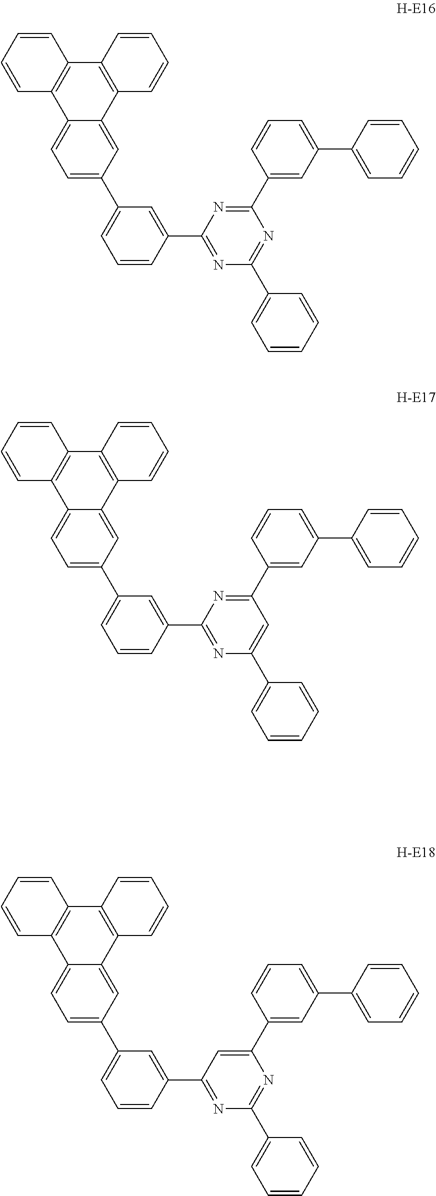 [SMILES: c1ccc(-c2cccc(-c3nc(-c4ccccc4)nc(-c4cccc(-c5ccc6c7ccccc7c7ccccc7c6c5)c4)n3)c2)cc1 ; c1ccc(-c2cccc(-c3cc(-c4ccccc4)nc(-c4cccc(-c5ccc6c7ccccc7c7ccccc7c6c5)c4)n3)c2)cc1 ; c1ccc(-c2cccc(-c3cc(-c4cccc(-c5ccc6c7ccccc7c7ccccc7c6c5)c4)nc(-c4ccccc4)n3)c2)cc1]

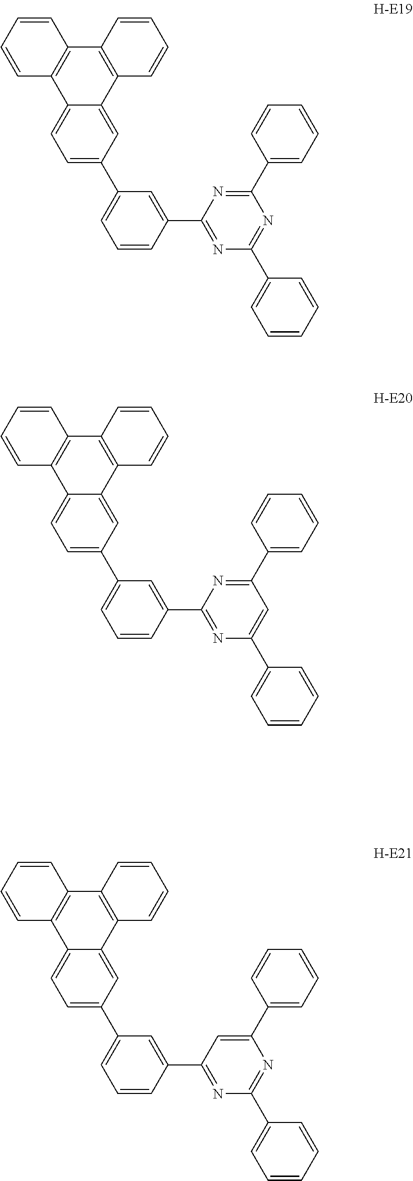 [SMILES: c1ccc(-c2nc(-c3ccccc3)nc(-c3cccc(-c4ccc5c6ccccc6c6ccccc6c5c4)c3)n2)cc1 ; c1ccc(-c2cc(-c3ccccc3)nc(-c3cccc(-c4ccc5c6ccccc6c6ccccc6c5c4)c3)n2)cc1 ; c1ccc(-c2cc(-c3cccc(-c4ccc5c6ccccc6c6ccccc6c5c4)c3)nc(-c3ccccc3)n2)cc1]

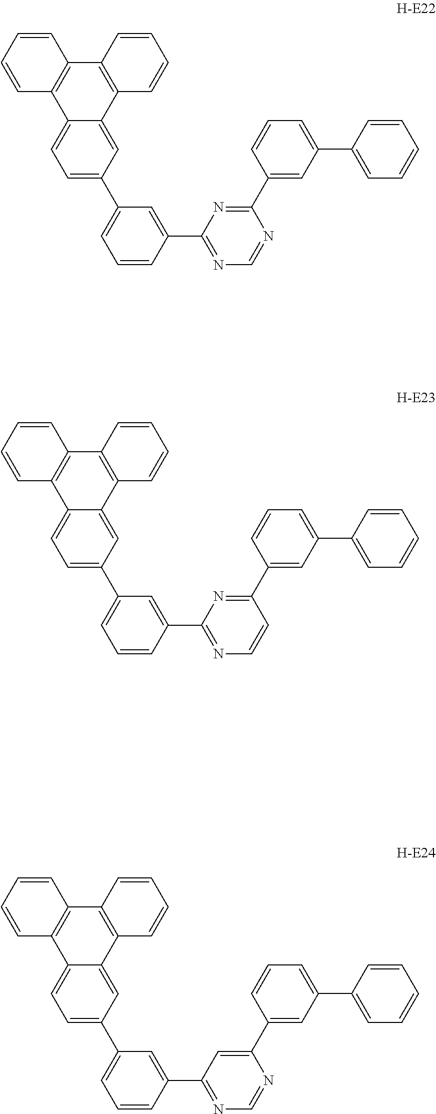 [SMILES: c1ccc(-c2cccc(-c3ncnc(-c4cccc(-c5ccc6c7ccccc7c7ccccc7c6c5)c4)n3)c2)cc1 ; c1ccc(-c2cccc(-c3ccnc(-c4cccc(-c5ccc6c7ccccc7c7ccccc7c6c5)c4)n3)c2)cc1 ; c1ccc(-c2cccc(-c3cc(-c4cccc(-c5ccc6c7ccccc7c7ccccc7c6c5)c4)ncn3)c2)cc1]

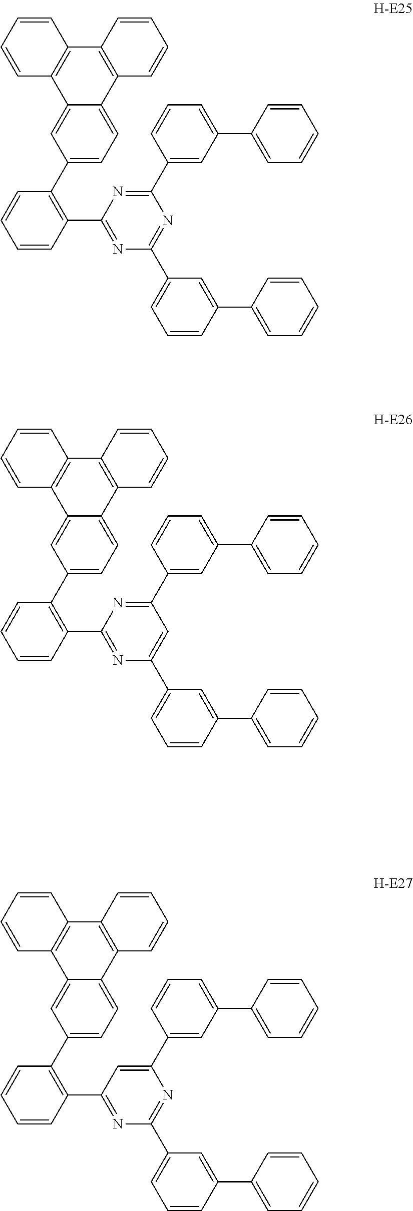 [SMILES: c1ccc(-c2cccc(-c3nc(-c4cccc(-c5ccccc5)c4)nc(-c4ccccc4-c4ccc5c6ccccc6c6ccccc6c5c4)n3)c2)cc1 ; c1ccc(-c2cccc(-c3cc(-c4cccc(-c5ccccc5)c4)nc(-c4ccccc4-c4ccc5c6ccccc6c6ccccc6c5c4)n3)c2)cc1 ; c1ccc(-c2cccc(-c3cc(-c4ccccc4-c4ccc5c6ccccc6c6ccccc6c5c4)nc(-c4cccc(-c5ccccc5)c4)n3)c2)cc1]

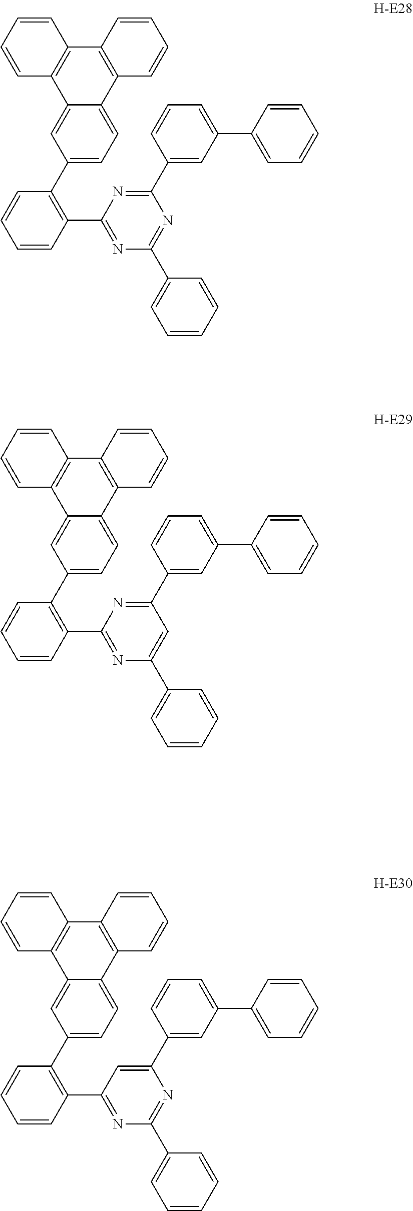 [SMILES: c1ccc(-c2cccc(-c3nc(-c4ccccc4)nc(-c4ccccc4-c4ccc5c6ccccc6c6ccccc6c5c4)n3)c2)cc1 ; c1ccc(-c2cccc(-c3cc(-c4ccccc4)nc(-c4ccccc4-c4ccc5c6ccccc6c6ccccc6c5c4)n3)c2)cc1 ; c1ccc(-c2cccc(-c3cc(-c4ccccc4-c4ccc5c6ccccc6c6ccccc6c5c4)nc(-c4ccccc4)n3)c2)cc1]

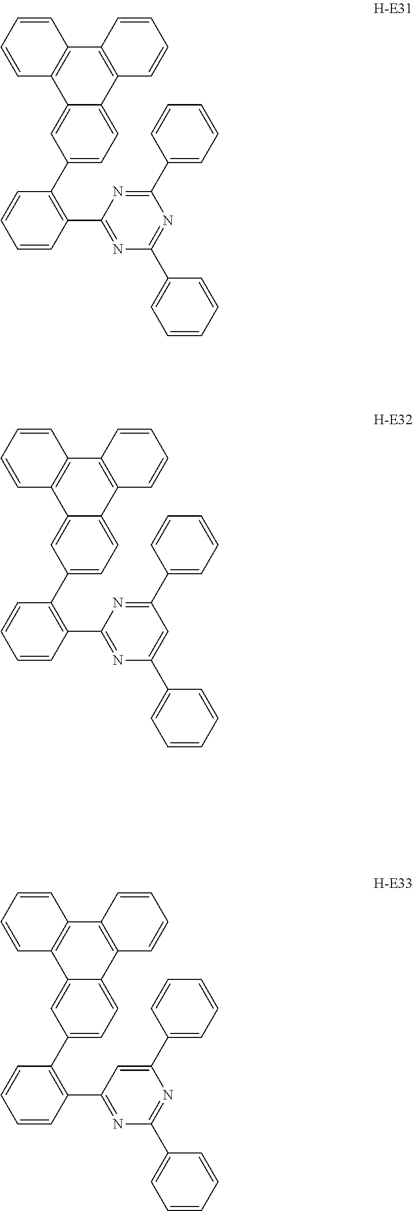 [SMILES: c1ccc(-c2nc(-c3ccccc3)nc(-c3ccccc3-c3ccc4c5ccccc5c5ccccc5c4c3)n2)cc1 ; c1ccc(-c2cc(-c3ccccc3)nc(-c3ccccc3-c3ccc4c5ccccc5c5ccccc5c4c3)n2)cc1 ; c1ccc(-c2cc(-c3ccccc3-c3ccc4c5ccccc5c5ccccc5c4c3)nc(-c3ccccc3)n2)cc1]

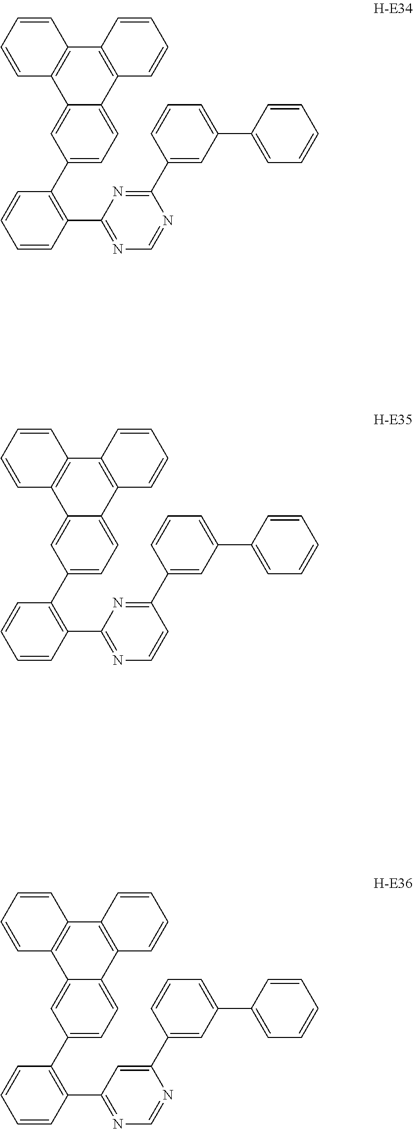 [SMILES: c1ccc(-c2cccc(-c3ncnc(-c4ccccc4-c4ccc5c6ccccc6c6ccccc6c5c4)n3)c2)cc1 ; c1ccc(-c2cccc(-c3ccnc(-c4ccccc4-c4ccc5c6ccccc6c6ccccc6c5c4)n3)c2)cc1 ; c1ccc(-c2cccc(-c3cc(-c4ccccc4-c4ccc5c6ccccc6c6ccccc6c5c4)ncn3)c2)cc1]

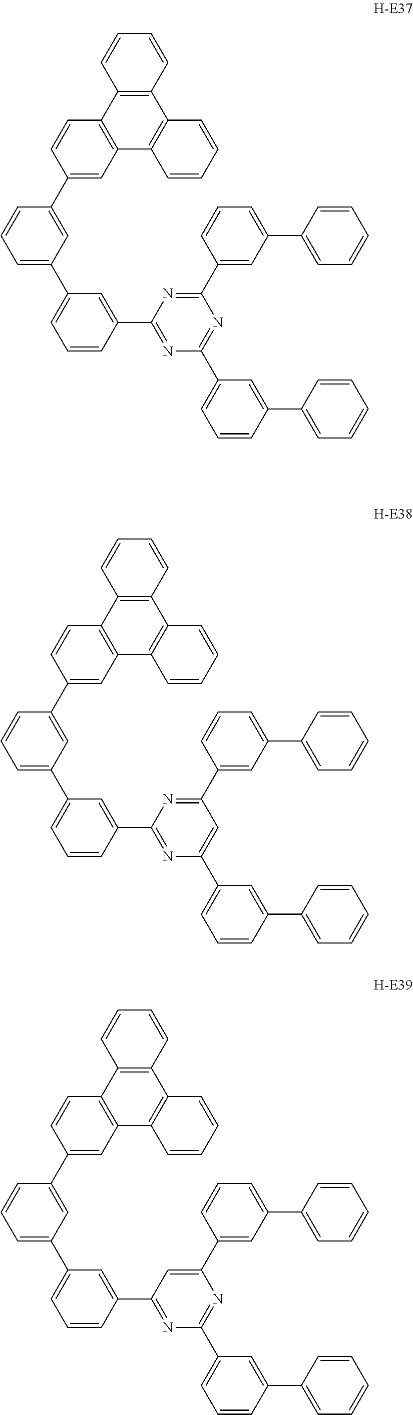 [SMILES: c1ccc(-c2cccc(-c3nc(-c4cccc(-c5ccccc5)c4)nc(-c4cccc(-c5cccc(-c6ccc7c8ccccc8c8ccccc8c7c6)c5)c4)n3)c2)cc1 ; c1ccc(-c2cccc(-c3cc(-c4cccc(-c5ccccc5)c4)nc(-c4cccc(-c5cccc(-c6ccc7c8ccccc8c8ccccc8c7c6)c5)c4)n3)c2)cc1 ; c1ccc(-c2cccc(-c3cc(-c4cccc(-c5cccc(-c6ccc7c8ccccc8c8ccccc8c7c6)c5)c4)nc(-c4cccc(-c5ccccc5)c4)n3)c2)cc1]

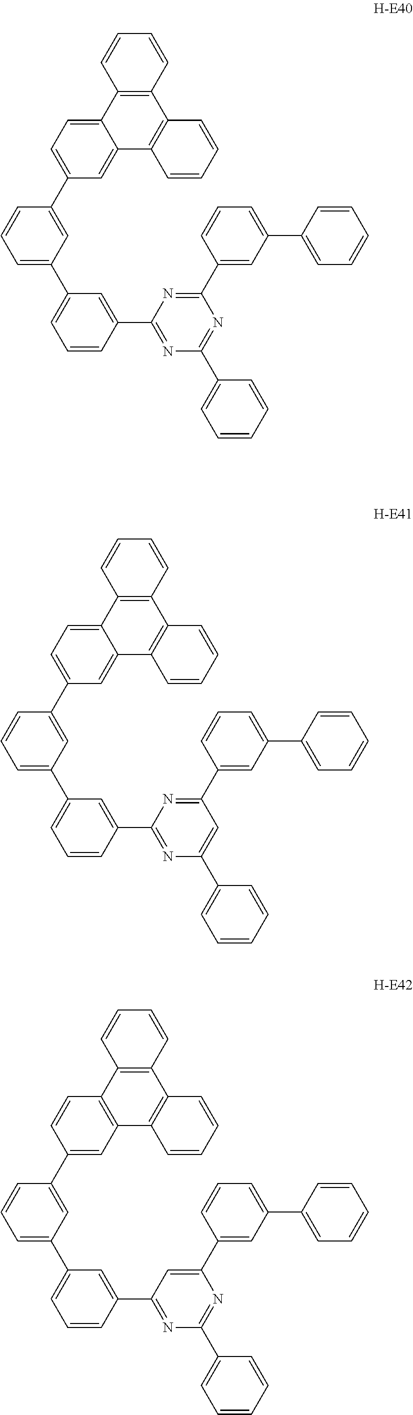 [SMILES: c1ccc(-c2cccc(-c3nc(-c4ccccc4)nc(-c4cccc(-c5cccc(-c6ccc7c8ccccc8c8ccccc8c7c6)c5)c4)n3)c2)cc1 ; c1ccc(-c2cccc(-c3cc(-c4ccccc4)nc(-c4cccc(-c5cccc(-c6ccc7c8ccccc8c8ccccc8c7c6)c5)c4)n3)c2)cc1 ; c1ccc(-c2cccc(-c3cc(-c4cccc(-c5cccc(-c6ccc7c8ccccc8c8ccccc8c7c6)c5)c4)nc(-c4ccccc4)n3)c2)cc1]

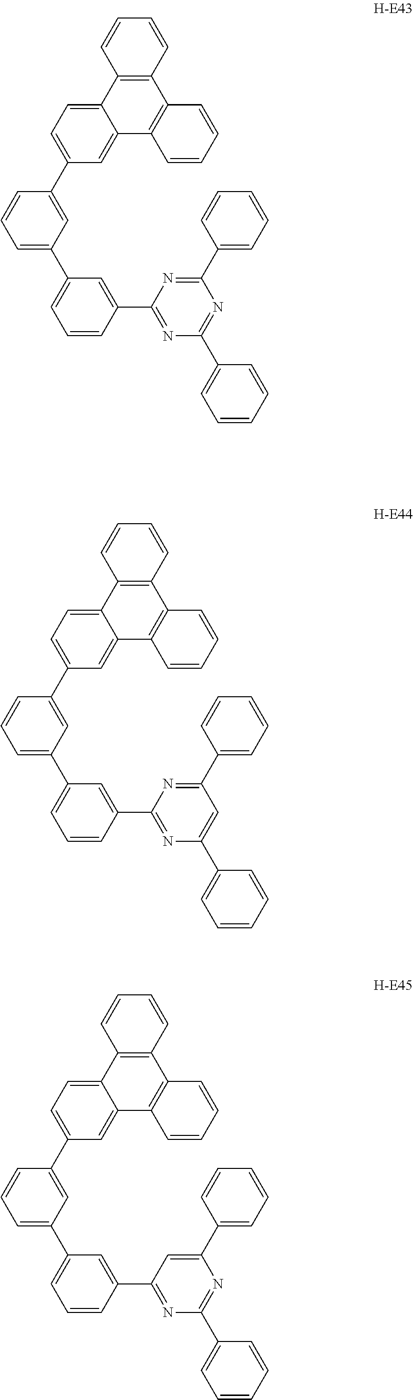 [SMILES: c1ccc(-c2nc(-c3ccccc3)nc(-c3cccc(-c4cccc(-c5ccc6c7ccccc7c7ccccc7c6c5)c4)c3)n2)cc1 ; c1ccc(-c2cc(-c3ccccc3)nc(-c3cccc(-c4cccc(-c5ccc6c7ccccc7c7ccccc7c6c5)c4)c3)n2)cc1 ; c1ccc(-c2cc(-c3cccc(-c4cccc(-c5ccc6c7ccccc7c7ccccc7c6c5)c4)c3)nc(-c3ccccc3)n2)cc1]

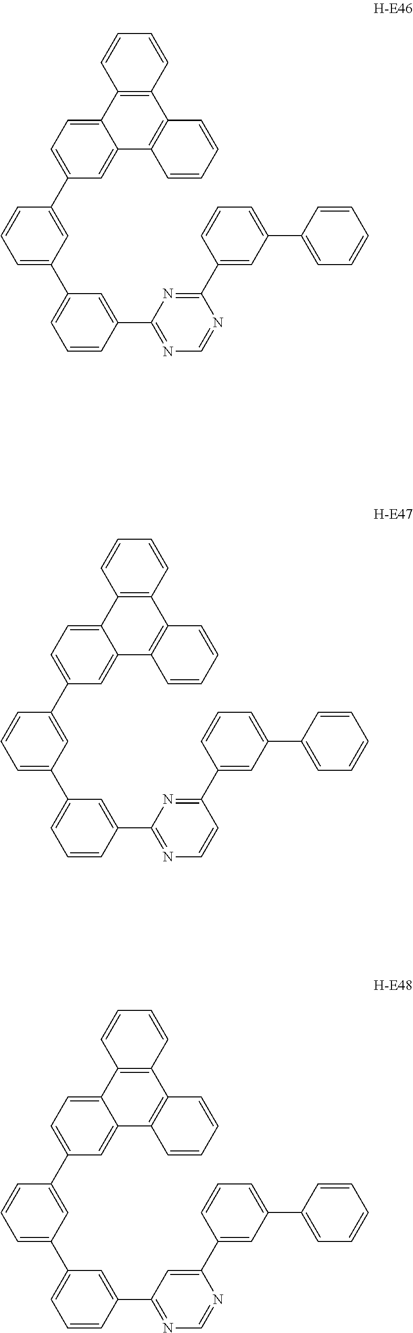 [SMILES: c1ccc(-c2cccc(-c3ncnc(-c4cccc(-c5cccc(-c6ccc7c8ccccc8c8ccccc8c7c6)c5)c4)n3)c2)cc1 ; c1ccc(-c2cccc(-c3ccnc(-c4cccc(-c5cccc(-c6ccc7c8ccccc8c8ccccc8c7c6)c5)c4)n3)c2)cc1 ; c1ccc(-c2cccc(-c3cc(-c4cccc(-c5cccc(-c6ccc7c8ccccc8c8ccccc8c7c6)c5)c4)ncn3)c2)cc1]

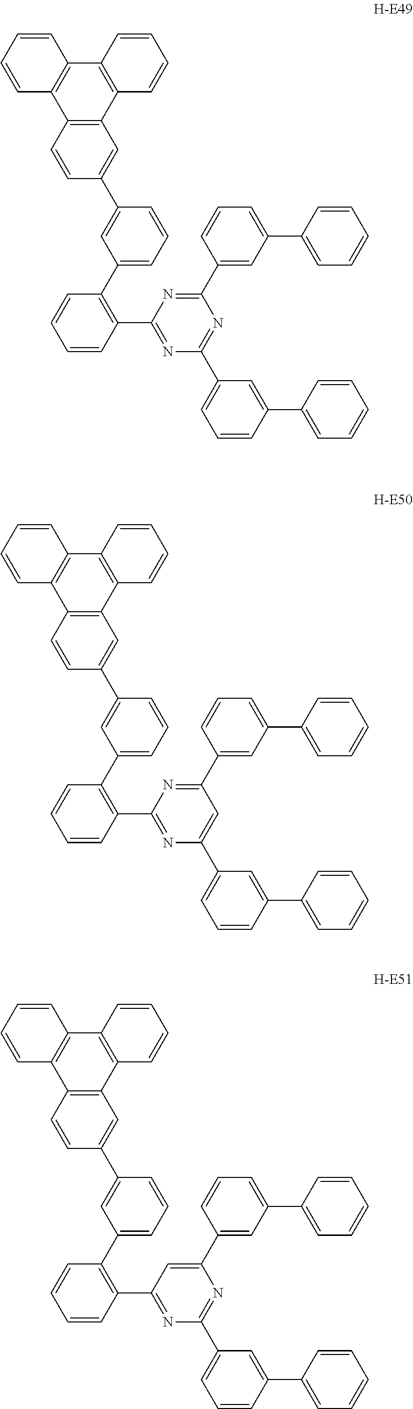 [SMILES: c1ccc(-c2cccc(-c3nc(-c4cccc(-c5ccccc5)c4)nc(-c4ccccc4-c4cccc(-c5ccc6c7ccccc7c7ccccc7c6c5)c4)n3)c2)cc1 ; c1ccc(-c2cccc(-c3cc(-c4cccc(-c5ccccc5)c4)nc(-c4ccccc4-c4cccc(-c5ccc6c7ccccc7c7ccccc7c6c5)c4)n3)c2)cc1 ; c1ccc(-c2cccc(-c3cc(-c4ccccc4-c4cccc(-c5ccc6c7ccccc7c7ccccc7c6c5)c4)nc(-c4cccc(-c5ccccc5)c4)n3)c2)cc1]

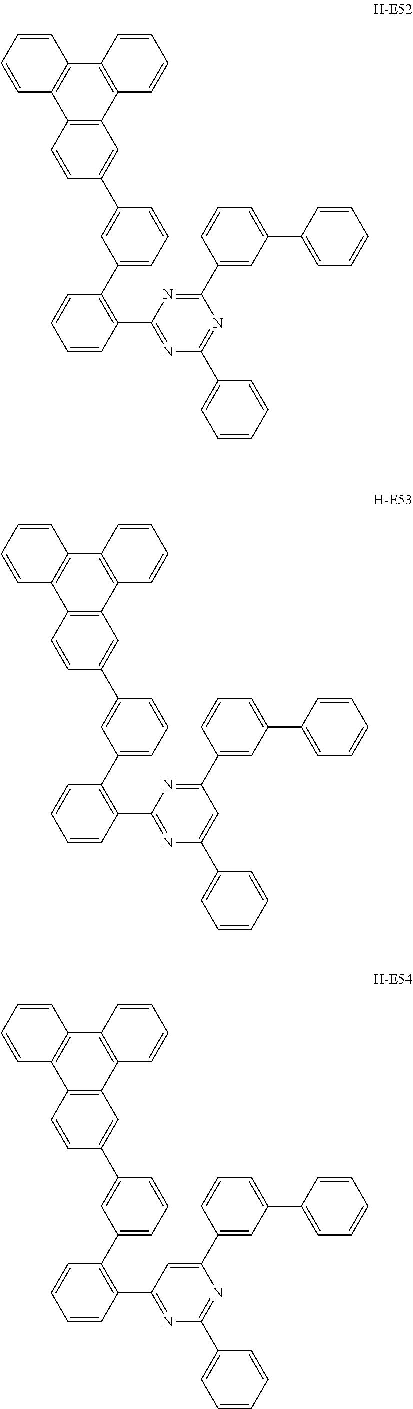 [SMILES: c1ccc(-c2cccc(-c3nc(-c4ccccc4)nc(-c4ccccc4-c4cccc(-c5ccc6c7ccccc7c7ccccc7c6c5)c4)n3)c2)cc1 ; c1ccc(-c2cccc(-c3cc(-c4ccccc4)nc(-c4ccccc4-c4cccc(-c5ccc6c7ccccc7c7ccccc7c6c5)c4)n3)c2)cc1 ; c1ccc(-c2cccc(-c3cc(-c4ccccc4-c4cccc(-c5ccc6c7ccccc7c7ccccc7c6c5)c4)nc(-c4ccccc4)n3)c2)cc1]

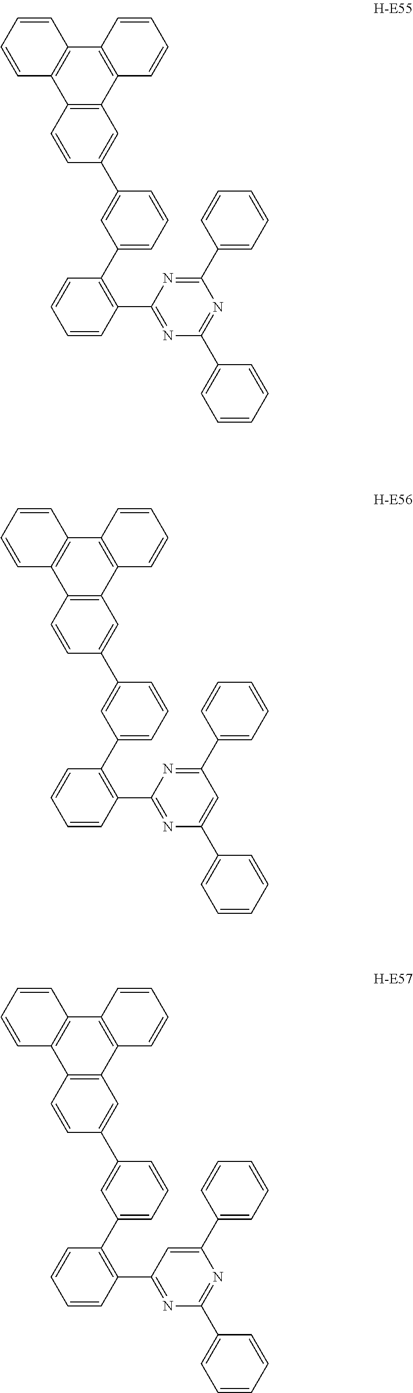 [SMILES: c1ccc(-c2nc(-c3ccccc3)nc(-c3ccccc3-c3cccc(-c4ccc5c6ccccc6c6ccccc6c5c4)c3)n2)cc1 ; c1ccc(-c2cc(-c3ccccc3)nc(-c3ccccc3-c3cccc(-c4ccc5c6ccccc6c6ccccc6c5c4)c3)n2)cc1 ; c1ccc(-c2cc(-c3ccccc3-c3cccc(-c4ccc5c6ccccc6c6ccccc6c5c4)c3)nc(-c3ccccc3)n2)cc1]

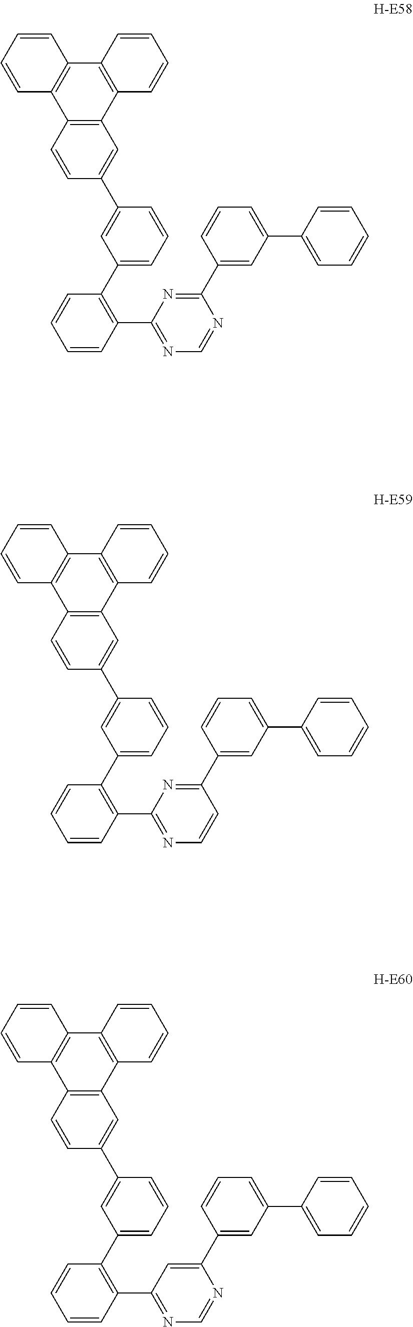 [SMILES: c1ccc(-c2cccc(-c3ncnc(-c4ccccc4-c4cccc(-c5ccc6c7ccccc7c7ccccc7c6c5)c4)n3)c2)cc1 ; c1ccc(-c2cccc(-c3ccnc(-c4ccccc4-c4cccc(-c5ccc6c7ccccc7c7ccccc7c6c5)c4)n3)c2)cc1 ; c1ccc(-c2cccc(-c3cc(-c4ccccc4-c4cccc(-c5ccc6c7ccccc7c7ccccc7c6c5)c4)ncn3)c2)cc1]

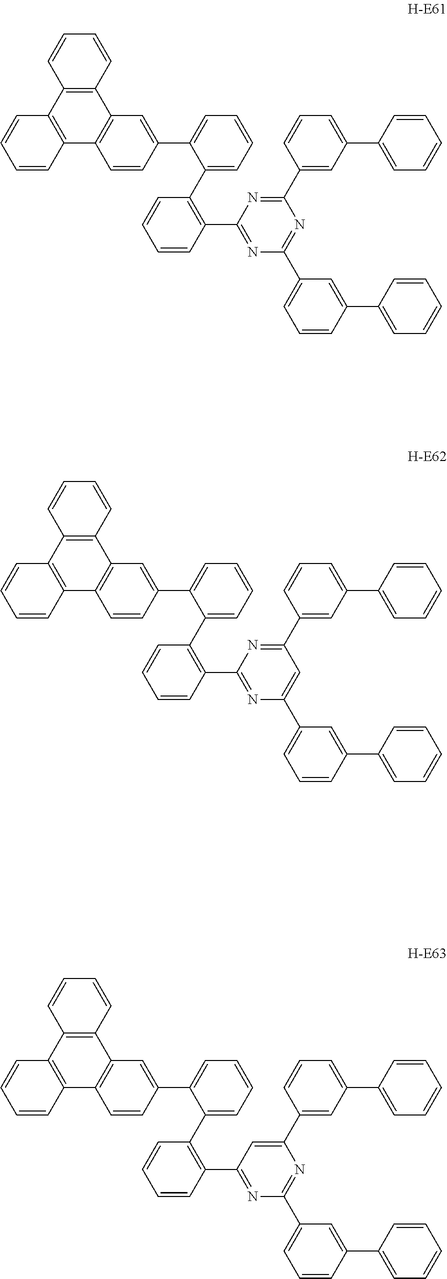 [SMILES: c1ccc(-c2cccc(-c3nc(-c4cccc(-c5ccccc5)c4)nc(-c4ccccc4-c4ccccc4-c4ccc5c6ccccc6c6ccccc6c5c4)n3)c2)cc1 ; c1ccc(-c2cccc(-c3cc(-c4cccc(-c5ccccc5)c4)nc(-c4ccccc4-c4ccccc4-c4ccc5c6ccccc6c6ccccc6c5c4)n3)c2)cc1 ; c1ccc(-c2cccc(-c3cc(-c4ccccc4-c4ccccc4-c4ccc5c6ccccc6c6ccccc6c5c4)nc(-c4cccc(-c5ccccc5)c4)n3)c2)cc1]

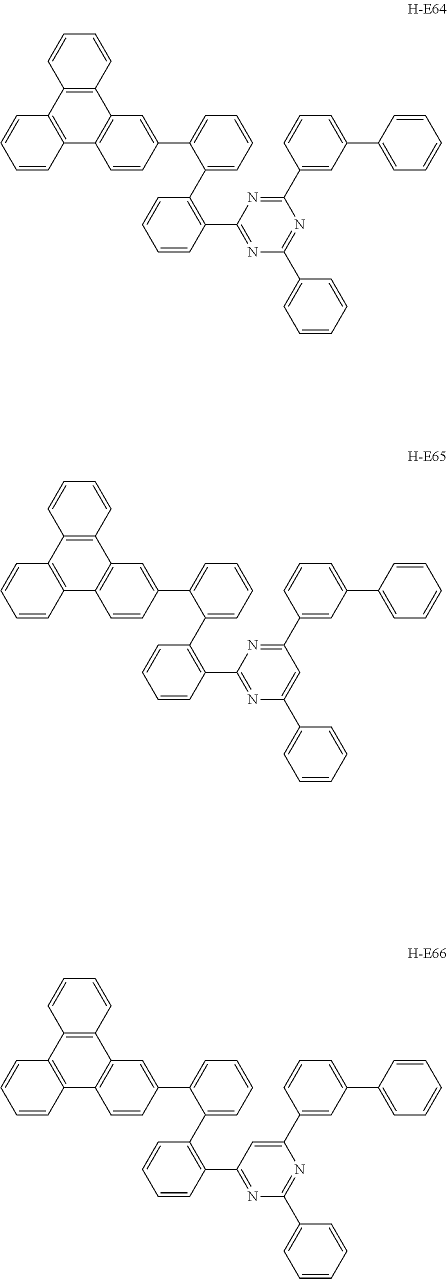 [SMILES: c1ccc(-c2cccc(-c3nc(-c4ccccc4)nc(-c4ccccc4-c4ccccc4-c4ccc5c6ccccc6c6ccccc6c5c4)n3)c2)cc1 ; c1ccc(-c2cccc(-c3cc(-c4ccccc4)nc(-c4ccccc4-c4ccccc4-c4ccc5c6ccccc6c6ccccc6c5c4)n3)c2)cc1 ; c1ccc(-c2cccc(-c3cc(-c4ccccc4-c4ccccc4-c4ccc5c6ccccc6c6ccccc6c5c4)nc(-c4ccccc4)n3)c2)cc1]

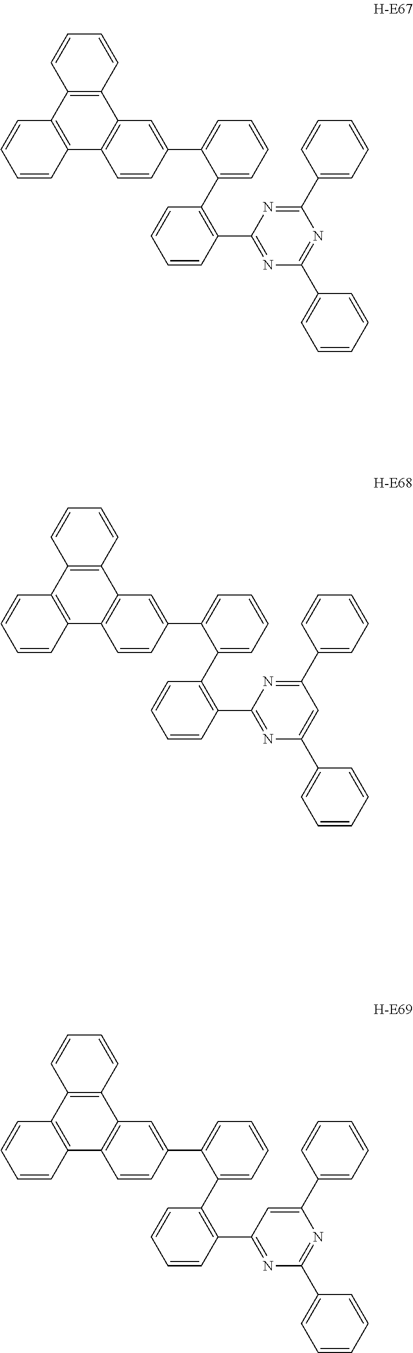 [SMILES: c1ccc(-c2nc(-c3ccccc3)nc(-c3ccccc3-c3ccccc3-c3ccc4c5ccccc5c5ccccc5c4c3)n2)cc1 ; c1ccc(-c2cc(-c3ccccc3)nc(-c3ccccc3-c3ccccc3-c3ccc4c5ccccc5c5ccccc5c4c3)n2)cc1 ; c1ccc(-c2cc(-c3ccccc3-c3ccccc3-c3ccc4c5ccccc5c5ccccc5c4c3)nc(-c3ccccc3)n2)cc1]

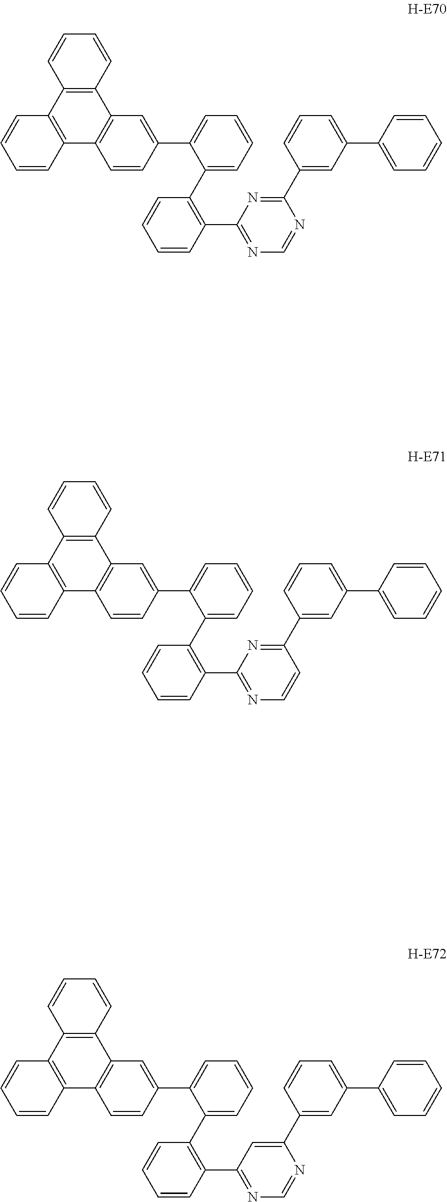 [SMILES: c1ccc(-c2cccc(-c3ncnc(-c4ccccc4-c4ccccc4-c4ccc5c6ccccc6c6ccccc6c5c4)n3)c2)cc1 ; c1ccc(-c2cccc(-c3ccnc(-c4ccccc4-c4ccccc4-c4ccc5c6ccccc6c6ccccc6c5c4)n3)c2)cc1 ; c1ccc(-c2cccc(-c3cc(-c4ccccc4-c4ccccc4-c4ccc5c6ccccc6c6ccccc6c5c4)ncn3)c2)cc1]

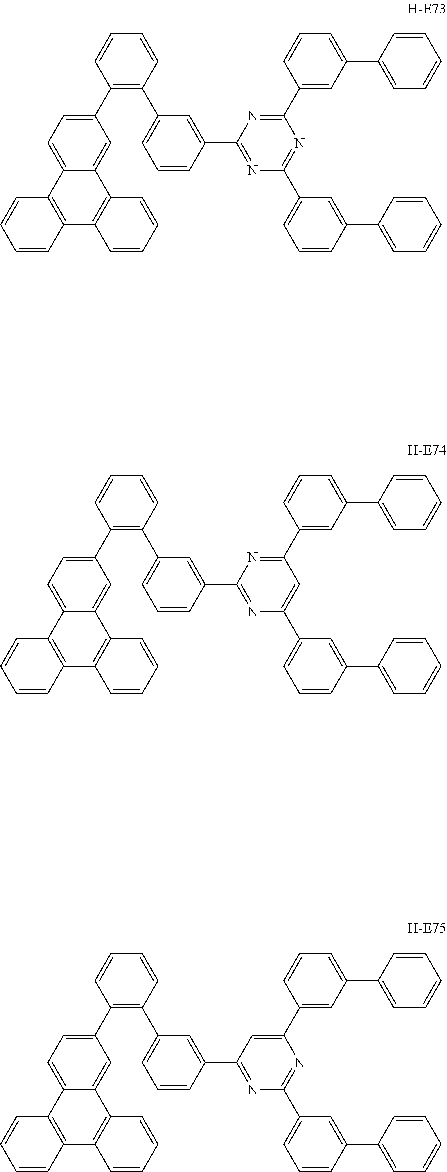 [SMILES: c1ccc(-c2cccc(-c3nc(-c4cccc(-c5ccccc5)c4)nc(-c4cccc(-c5ccccc5-c5ccc6c7ccccc7c7ccccc7c6c5)c4)n3)c2)cc1 ; c1ccc(-c2cccc(-c3cc(-c4cccc(-c5ccccc5)c4)nc(-c4cccc(-c5ccccc5-c5ccc6c7ccccc7c7ccccc7c6c5)c4)n3)c2)cc1 ; c1ccc(-c2cccc(-c3cc(-c4cccc(-c5ccccc5-c5ccc6c7ccccc7c7ccccc7c6c5)c4)nc(-c4cccc(-c5ccccc5)c4)n3)c2)cc1]

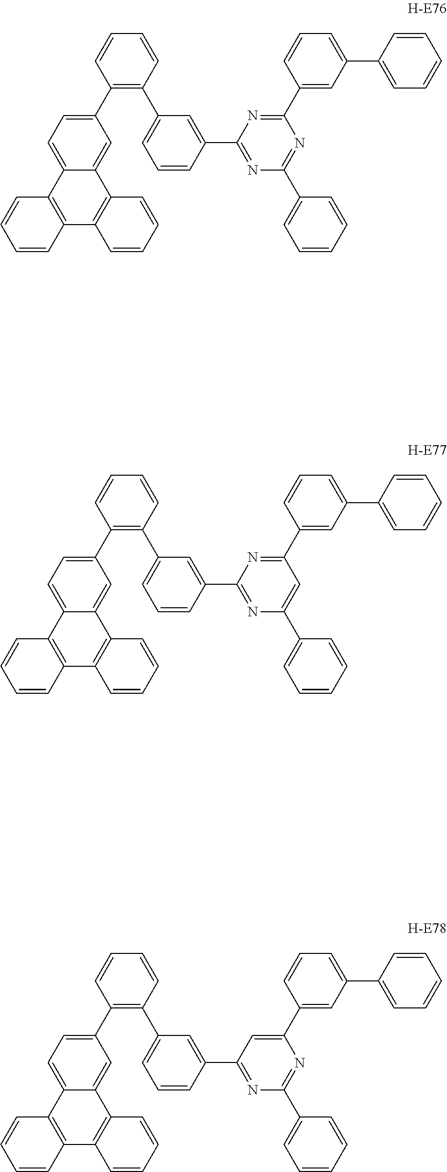 [SMILES: c1ccc(-c2cccc(-c3nc(-c4ccccc4)nc(-c4cccc(-c5ccccc5-c5ccc6c7ccccc7c7ccccc7c6c5)c4)n3)c2)cc1 ; c1ccc(-c2cccc(-c3cc(-c4ccccc4)nc(-c4cccc(-c5ccccc5-c5ccc6c7ccccc7c7ccccc7c6c5)c4)n3)c2)cc1 ; c1ccc(-c2cccc(-c3cc(-c4cccc(-c5ccccc5-c5ccc6c7ccccc7c7ccccc7c6c5)c4)nc(-c4ccccc4)n3)c2)cc1]

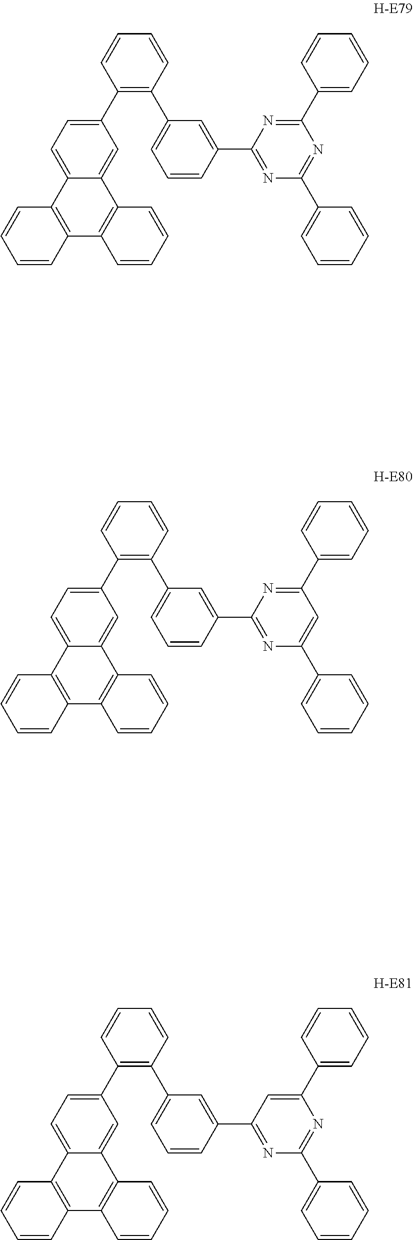 [SMILES: c1ccc(-c2nc(-c3ccccc3)nc(-c3cccc(-c4ccccc4-c4ccc5c6ccccc6c6ccccc6c5c4)c3)n2)cc1 ; c1ccc(-c2cc(-c3ccccc3)nc(-c3cccc(-c4ccccc4-c4ccc5c6ccccc6c6ccccc6c5c4)c3)n2)cc1 ; c1ccc(-c2cc(-c3cccc(-c4ccccc4-c4ccc5c6ccccc6c6ccccc6c5c4)c3)nc(-c3ccccc3)n2)cc1]

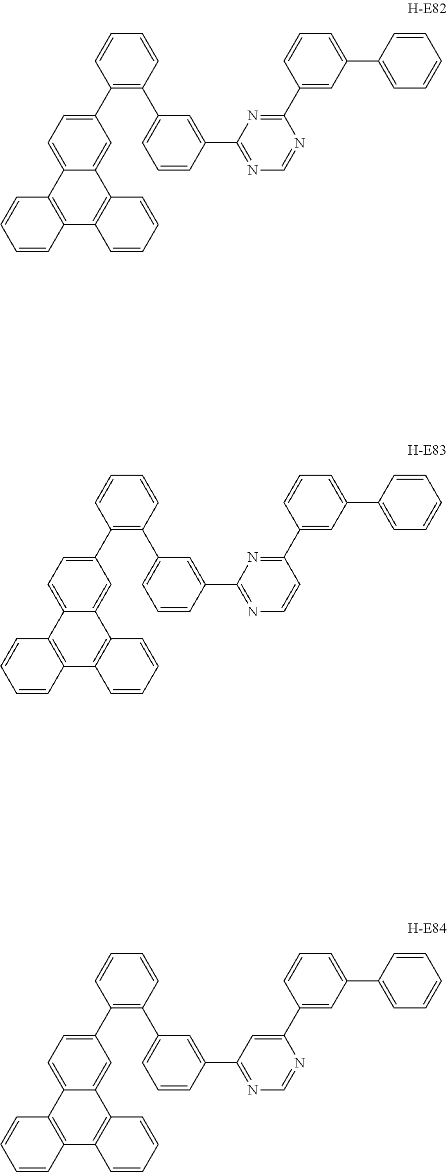 [SMILES: c1ccc(-c2cccc(-c3ncnc(-c4cccc(-c5ccccc5-c5ccc6c7ccccc7c7ccccc7c6c5)c4)n3)c2)cc1 ; c1ccc(-c2cccc(-c3ccnc(-c4cccc(-c5ccccc5-c5ccc6c7ccccc7c7ccccc7c6c5)c4)n3)c2)cc1 ; c1ccc(-c2cccc(-c3cc(-c4cccc(-c5ccccc5-c5ccc6c7ccccc7c7ccccc7c6c5)c4)ncn3)c2)cc1]

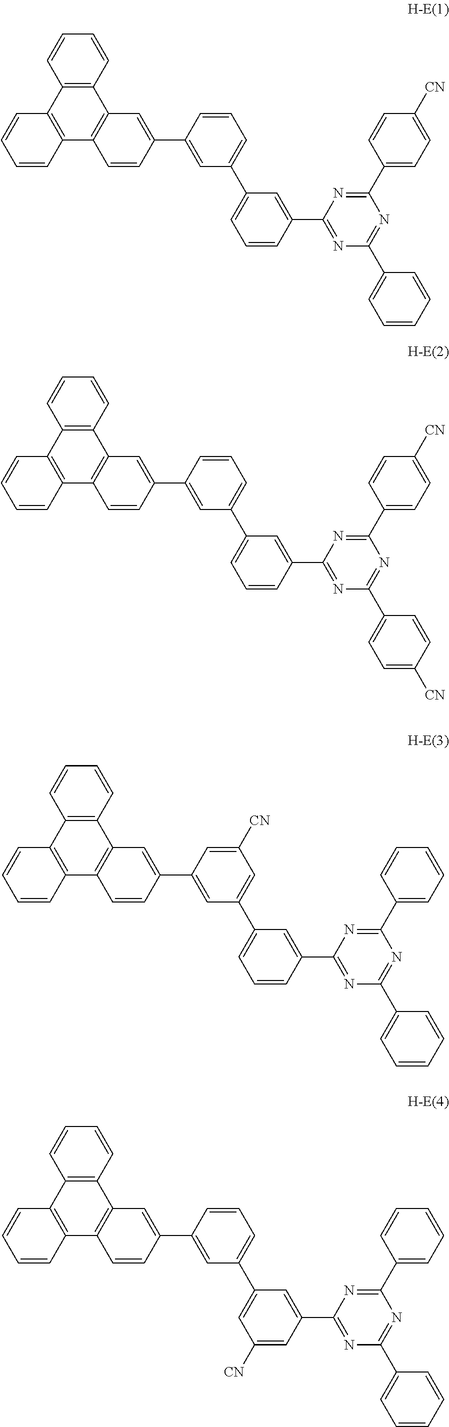

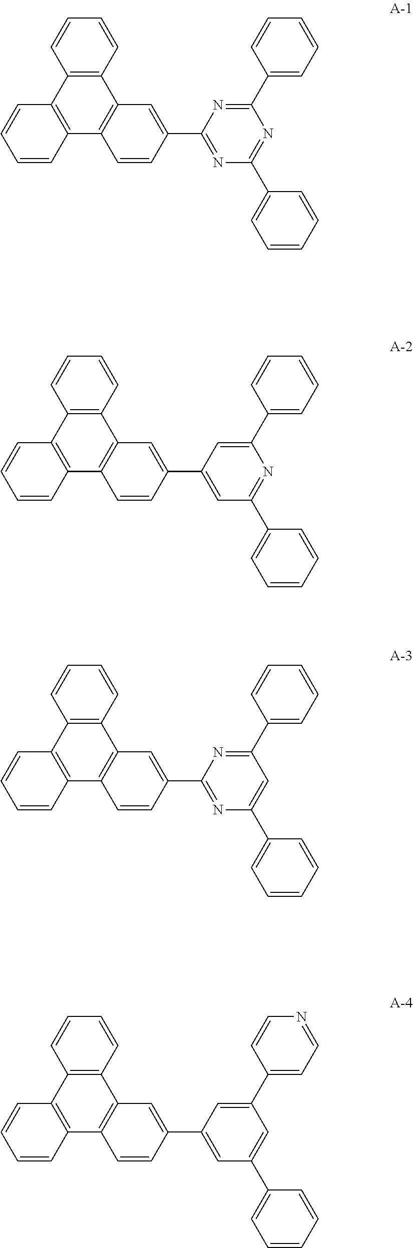 [SMILES: c1ccc(-c2nc(-c3ccccc3)nc(-c3ccc4c5ccccc5c5ccccc5c4c3)n2)cc1 ; c1ccc(-c2cc(-c3ccc4c5ccccc5c5ccccc5c4c3)cc(-c3ccccc3)n2)cc1 ; c1ccc(-c2cc(-c3ccccc3)nc(-c3ccc4c5ccccc5c5ccccc5c4c3)n2)cc1 ; c1ccc(-c2cc(-c3ccncc3)cc(-c3ccc4c5ccccc5c5ccccc5c4c3)c2)cc1]

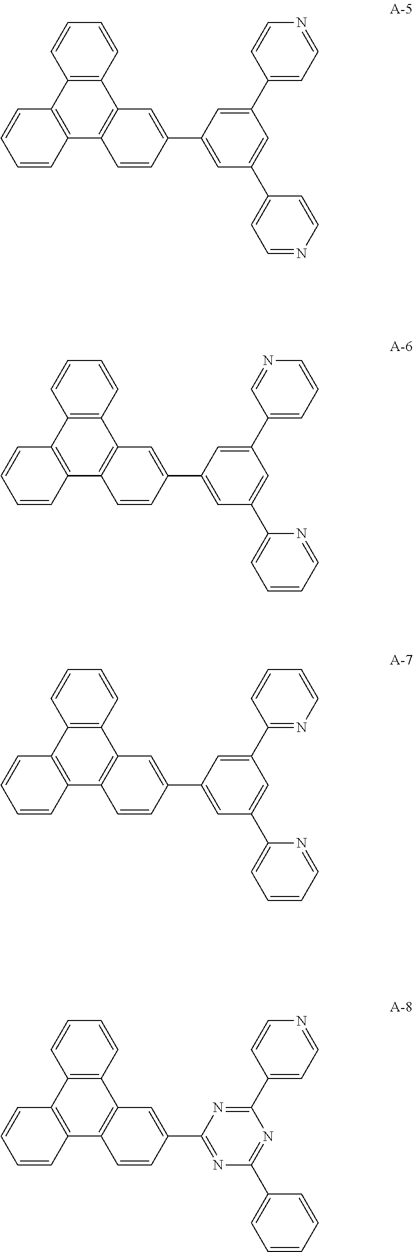 [SMILES: c1ccc2c(c1)c1ccccc1c1cc(-c3cc(-c4ccncc4)cc(-c4ccncc4)c3)ccc21 ; c1ccc(-c2cc(-c3cccnc3)cc(-c3ccc4c5ccccc5c5ccccc5c4c3)c2)nc1 ; c1ccc(-c2cc(-c3ccc4c5ccccc5c5ccccc5c4c3)cc(-c3ccccn3)c2)nc1 ; c1ccc(-c2nc(-c3ccncc3)nc(-c3ccc4c5ccccc5c5ccccc5c4c3)n2)cc1]

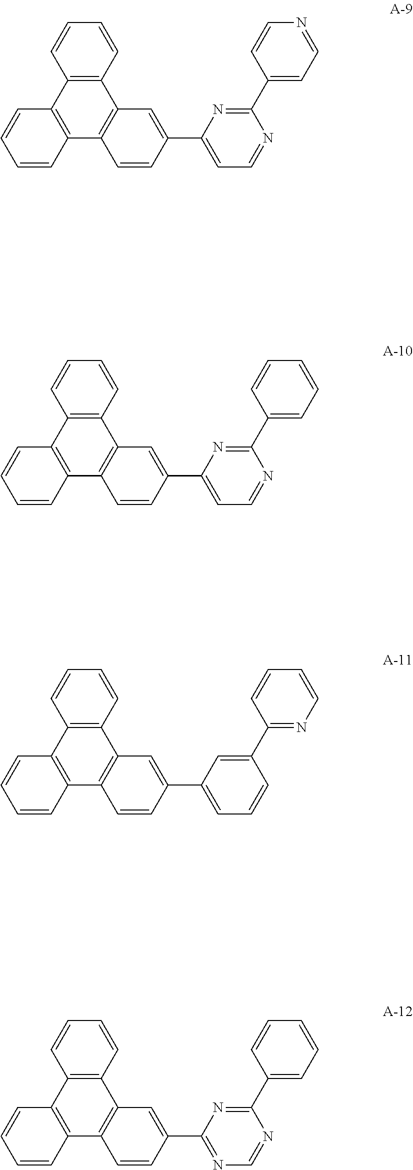 [SMILES: c1ccc2c(c1)c1ccccc1c1cc(-c3ccnc(-c4ccncc4)n3)ccc21 ; c1ccc(-c2nccc(-c3ccc4c5ccccc5c5ccccc5c4c3)n2)cc1 ; c1ccc(-c2cccc(-c3ccc4c5ccccc5c5ccccc5c4c3)c2)nc1 ; c1ccc(-c2ncnc(-c3ccc4c5ccccc5c5ccccc5c4c3)n2)cc1]

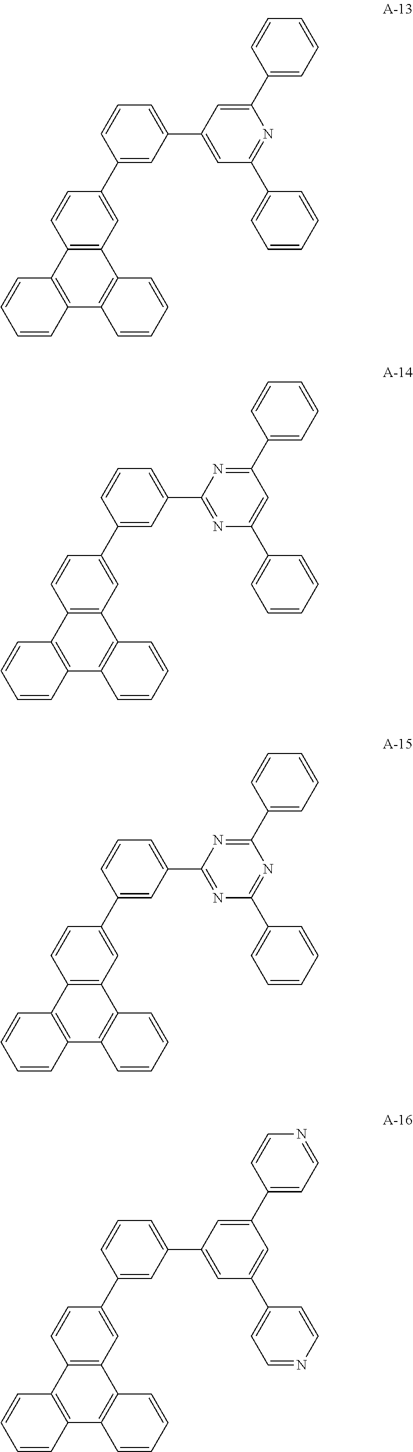 [SMILES: c1ccc(-c2cc(-c3cccc(-c4ccc5c6ccccc6c6ccccc6c5c4)c3)cc(-c3ccccc3)n2)cc1 ; c1ccc(-c2cc(-c3ccccc3)nc(-c3cccc(-c4ccc5c6ccccc6c6ccccc6c5c4)c3)n2)cc1 ; c1ccc(-c2nc(-c3ccccc3)nc(-c3cccc(-c4ccc5c6ccccc6c6ccccc6c5c4)c3)n2)cc1 ; c1cc(-c2cc(-c3ccncc3)cc(-c3ccncc3)c2)cc(-c2ccc3c4ccccc4c4ccccc4c3c2)c1]

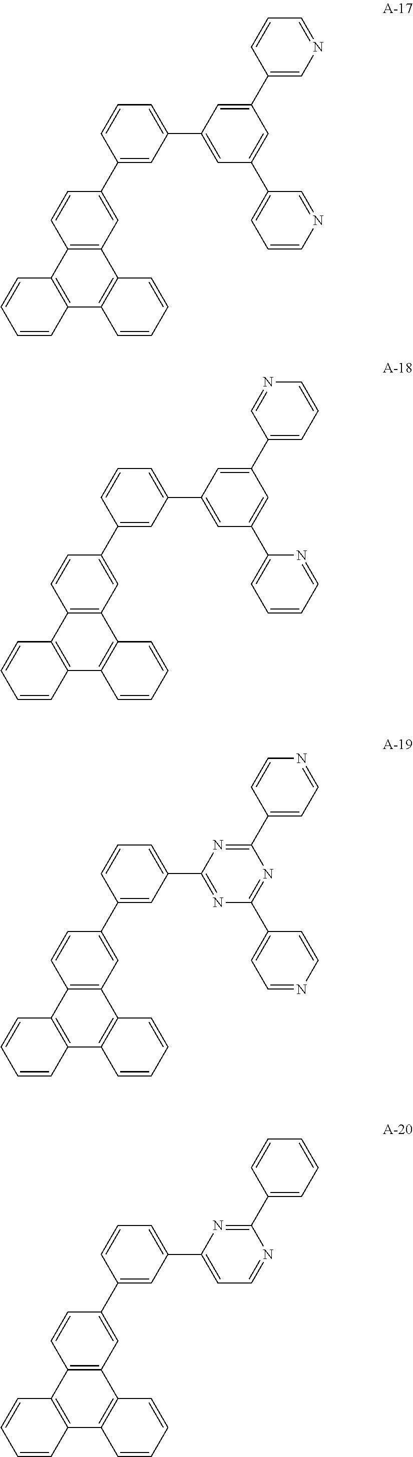 [SMILES: c1cncc(-c2cc(-c3cccnc3)cc(-c3cccc(-c4ccc5c6ccccc6c6ccccc6c5c4)c3)c2)c1 ; c1ccc(-c2cc(-c3cccnc3)cc(-c3cccc(-c4ccc5c6ccccc6c6ccccc6c5c4)c3)c2)nc1 ; c1cc(-c2ccc3c4ccccc4c4ccccc4c3c2)cc(-c2nc(-c3ccncc3)nc(-c3ccncc3)n2)c1 ; c1ccc(-c2nccc(-c3cccc(-c4ccc5c6ccccc6c6ccccc6c5c4)c3)n2)cc1]

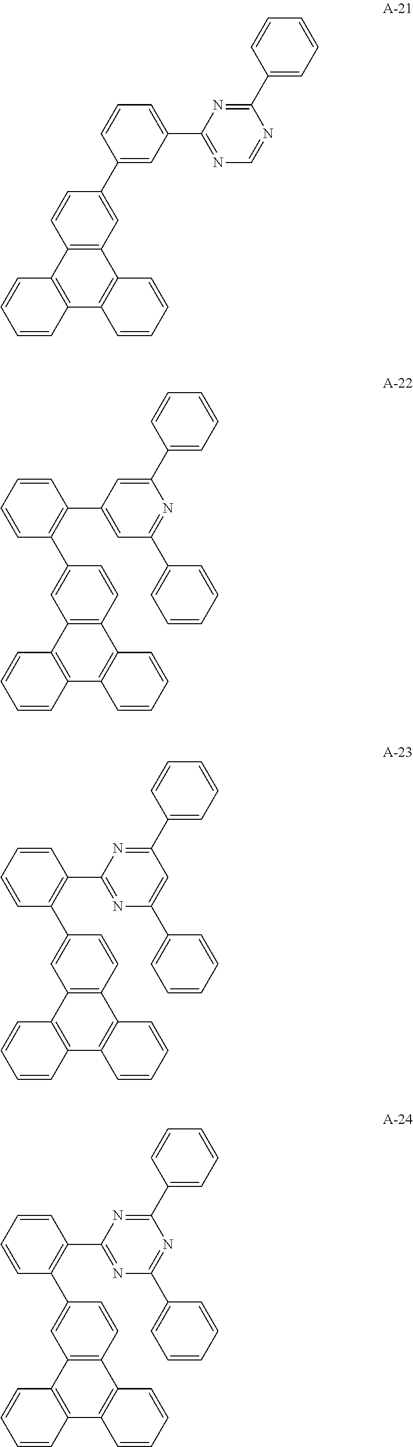 [SMILES: c1ccc(-c2ncnc(-c3cccc(-c4ccc5c6ccccc6c6ccccc6c5c4)c3)n2)cc1 ; c1ccc(-c2cc(-c3ccccc3-c3ccc4c5ccccc5c5ccccc5c4c3)cc(-c3ccccc3)n2)cc1 ; c1ccc(-c2cc(-c3ccccc3)nc(-c3ccccc3-c3ccc4c5ccccc5c5ccccc5c4c3)n2)cc1 ; c1ccc(-c2nc(-c3ccccc3)nc(-c3ccccc3-c3ccc4c5ccccc5c5ccccc5c4c3)n2)cc1]

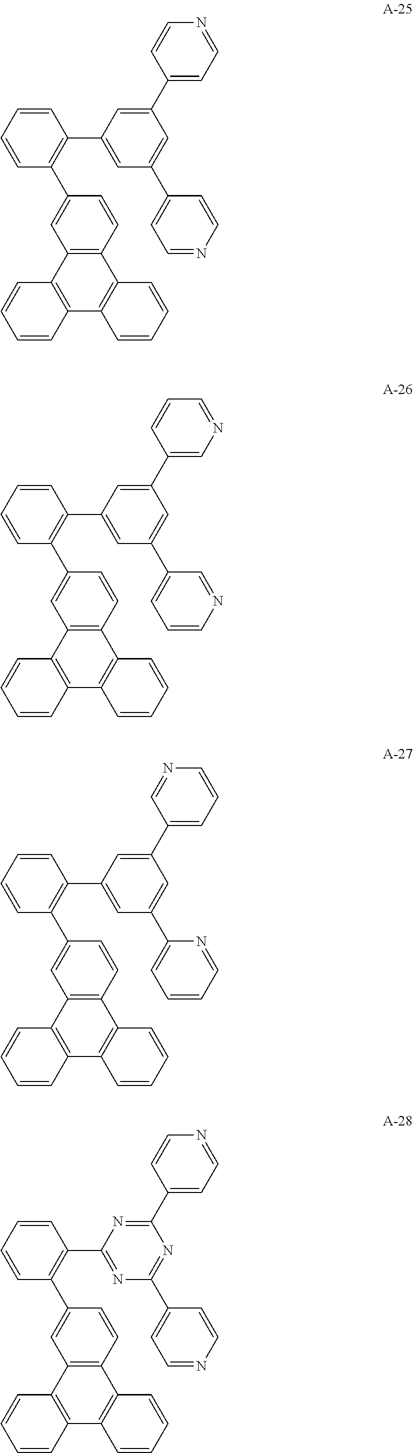 [SMILES: c1ccc(-c2ccc3c4ccccc4c4ccccc4c3c2)c(-c2cc(-c3ccncc3)cc(-c3ccncc3)c2)c1 ; c1cncc(-c2cc(-c3cccnc3)cc(-c3ccccc3-c3ccc4c5ccccc5c5ccccc5c4c3)c2)c1 ; c1ccc(-c2cc(-c3cccnc3)cc(-c3ccccc3-c3ccc4c5ccccc5c5ccccc5c4c3)c2)nc1 ; c1ccc(-c2nc(-c3ccncc3)nc(-c3ccncc3)n2)c(-c2ccc3c4ccccc4c4ccccc4c3c2)c1]

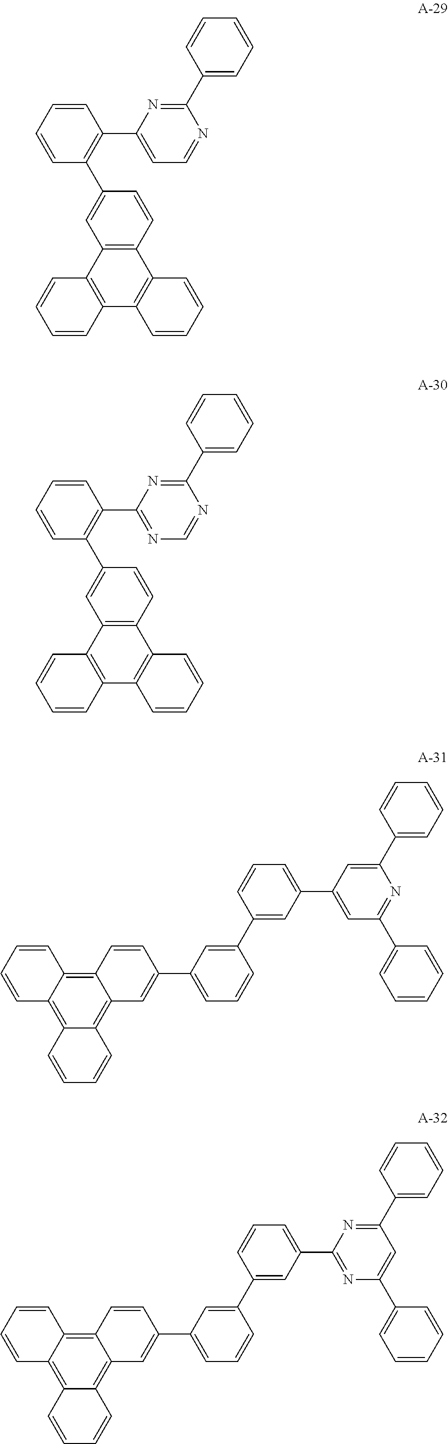 [SMILES: c1ccc(-c2nccc(-c3ccccc3-c3ccc4c5ccccc5c5ccccc5c4c3)n2)cc1 ; c1ccc(-c2ncnc(-c3ccccc3-c3ccc4c5ccccc5c5ccccc5c4c3)n2)cc1 ; c1ccc(-c2cc(-c3cccc(-c4cccc(-c5ccc6c7ccccc7c7ccccc7c6c5)c4)c3)cc(-c3ccccc3)n2)cc1 ; c1ccc(-c2cc(-c3ccccc3)nc(-c3cccc(-c4cccc(-c5ccc6c7ccccc7c7ccccc7c6c5)c4)c3)n2)cc1]

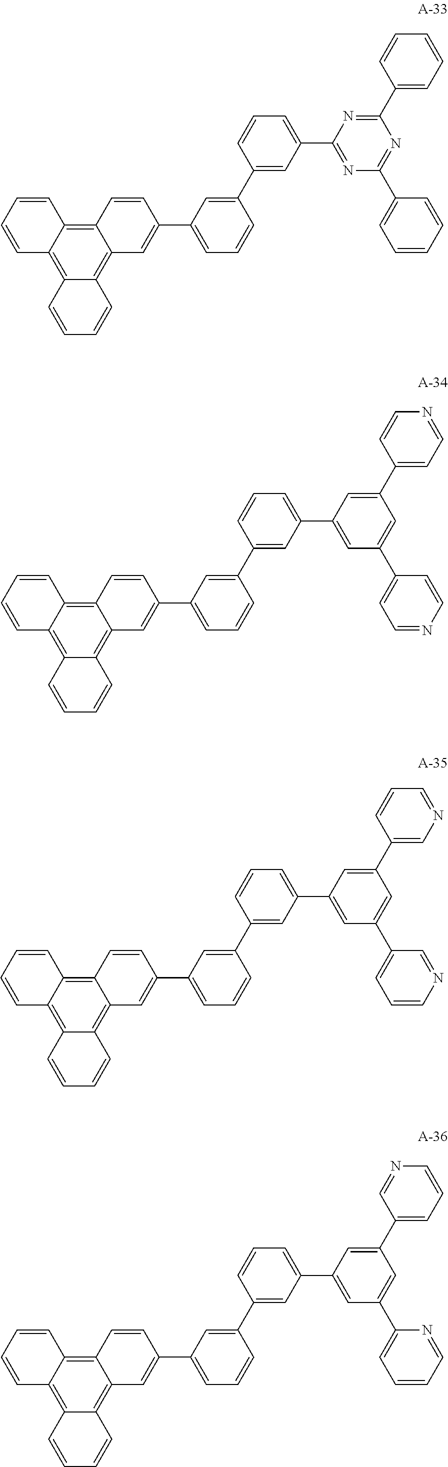 [SMILES: c1ccc(-c2nc(-c3ccccc3)nc(-c3cccc(-c4cccc(-c5ccc6c7ccccc7c7ccccc7c6c5)c4)c3)n2)cc1 ; c1cc(-c2cccc(-c3ccc4c5ccccc5c5ccccc5c4c3)c2)cc(-c2cc(-c3ccncc3)cc(-c3ccncc3)c2)c1 ; c1cncc(-c2cc(-c3cccnc3)cc(-c3cccc(-c4cccc(-c5ccc6c7ccccc7c7ccccc7c6c5)c4)c3)c2)c1 ; c1ccc(-c2cc(-c3cccnc3)cc(-c3cccc(-c4cccc(-c5ccc6c7ccccc7c7ccccc7c6c5)c4)c3)c2)nc1]

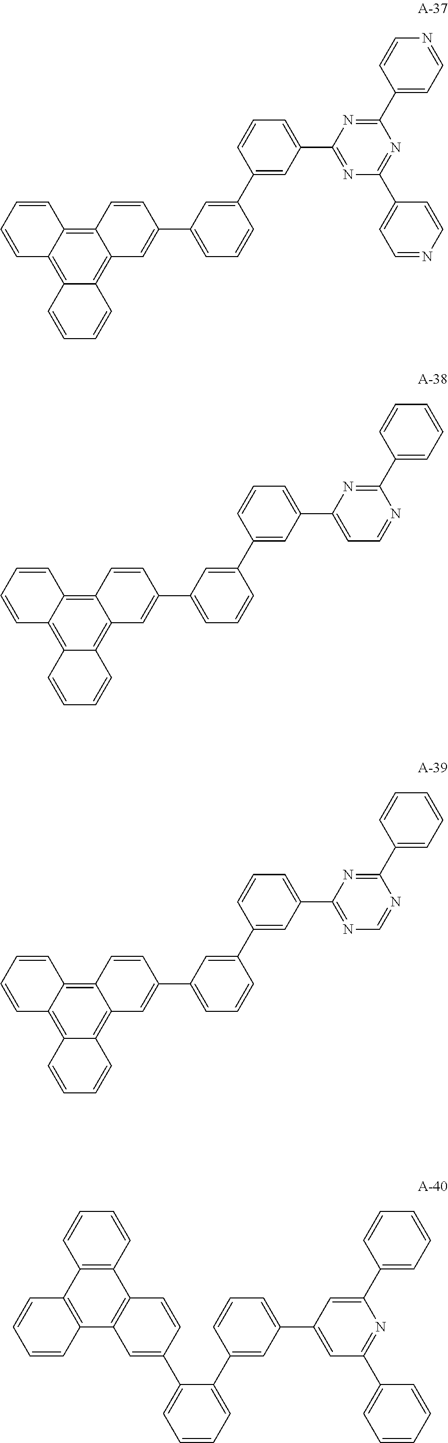 [SMILES: c1cc(-c2cccc(-c3nc(-c4ccncc4)nc(-c4ccncc4)n3)c2)cc(-c2ccc3c4ccccc4c4ccccc4c3c2)c1 ; c1ccc(-c2nccc(-c3cccc(-c4cccc(-c5ccc6c7ccccc7c7ccccc7c6c5)c4)c3)n2)cc1 ; c1ccc(-c2ncnc(-c3cccc(-c4cccc(-c5ccc6c7ccccc7c7ccccc7c6c5)c4)c3)n2)cc1 ; c1ccc(-c2cc(-c3cccc(-c4ccccc4-c4ccc5c6ccccc6c6ccccc6c5c4)c3)cc(-c3ccccc3)n2)cc1]

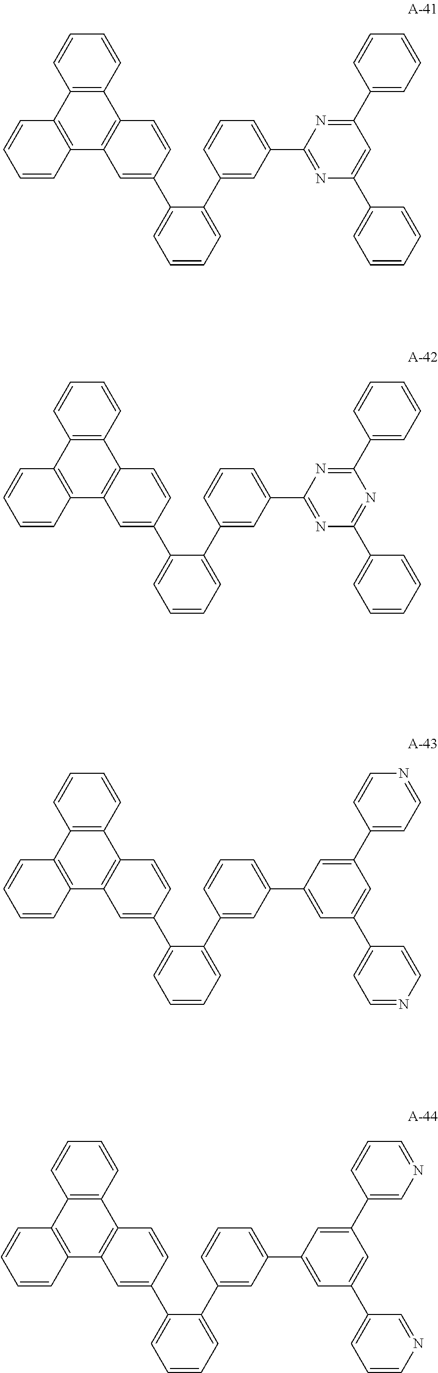 [SMILES: c1ccc(-c2cc(-c3ccccc3)nc(-c3cccc(-c4ccccc4-c4ccc5c6ccccc6c6ccccc6c5c4)c3)n2)cc1 ; c1ccc(-c2nc(-c3ccccc3)nc(-c3cccc(-c4ccccc4-c4ccc5c6ccccc6c6ccccc6c5c4)c3)n2)cc1 ; c1cc(-c2cc(-c3ccncc3)cc(-c3ccncc3)c2)cc(-c2ccccc2-c2ccc3c4ccccc4c4ccccc4c3c2)c1 ; c1cncc(-c2cc(-c3cccnc3)cc(-c3cccc(-c4ccccc4-c4ccc5c6ccccc6c6ccccc6c5c4)c3)c2)c1]

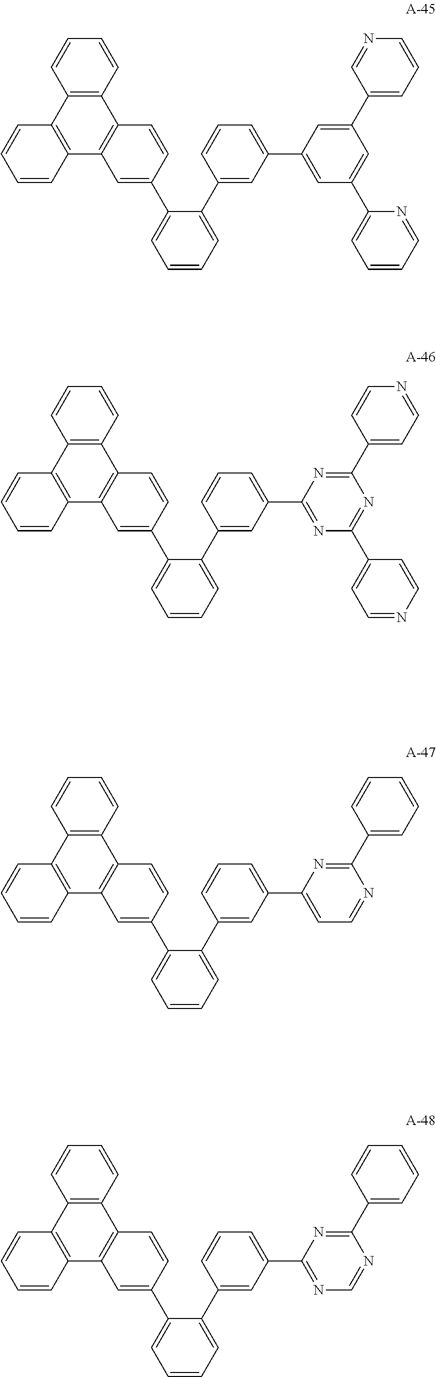 [SMILES: c1ccc(-c2cc(-c3cccnc3)cc(-c3cccc(-c4ccccc4-c4ccc5c6ccccc6c6ccccc6c5c4)c3)c2)nc1 ; c1cc(-c2nc(-c3ccncc3)nc(-c3ccncc3)n2)cc(-c2ccccc2-c2ccc3c4ccccc4c4ccccc4c3c2)c1 ; c1ccc(-c2nccc(-c3cccc(-c4ccccc4-c4ccc5c6ccccc6c6ccccc6c5c4)c3)n2)cc1 ; c1ccc(-c2ncnc(-c3cccc(-c4ccccc4-c4ccc5c6ccccc6c6ccccc6c5c4)c3)n2)cc1]

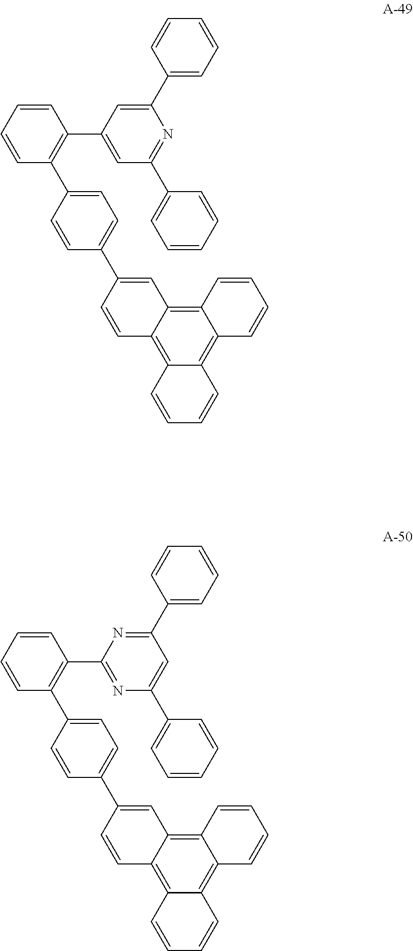 [SMILES: c1ccc(-c2cc(-c3ccccc3-c3ccc(-c4ccc5c6ccccc6c6ccccc6c5c4)cc3)cc(-c3ccccc3)n2)cc1 ; c1ccc(-c2cc(-c3ccccc3)nc(-c3ccccc3-c3ccc(-c4ccc5c6ccccc6c6ccccc6c5c4)cc3)n2)cc1]

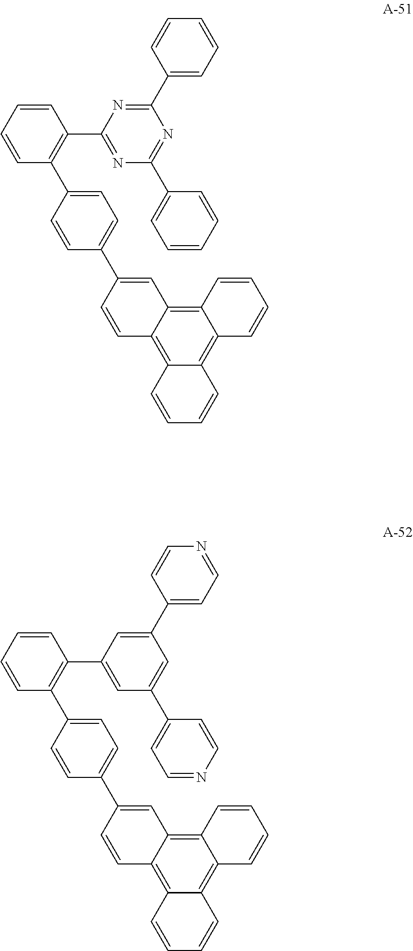 [SMILES: c1ccc(-c2nc(-c3ccccc3)nc(-c3ccccc3-c3ccc(-c4ccc5c6ccccc6c6ccccc6c5c4)cc3)n2)cc1 ; c1ccc(-c2cc(-c3ccncc3)cc(-c3ccncc3)c2)c(-c2ccc(-c3ccc4c5ccccc5c5ccccc5c4c3)cc2)c1]

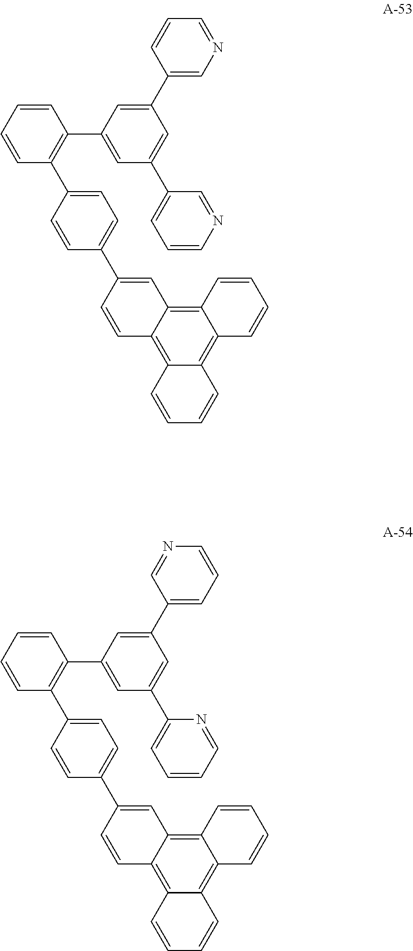 [SMILES: c1cncc(-c2cc(-c3cccnc3)cc(-c3ccccc3-c3ccc(-c4ccc5c6ccccc6c6ccccc6c5c4)cc3)c2)c1 ; c1ccc(-c2cc(-c3cccnc3)cc(-c3ccccc3-c3ccc(-c4ccc5c6ccccc6c6ccccc6c5c4)cc3)c2)nc1]

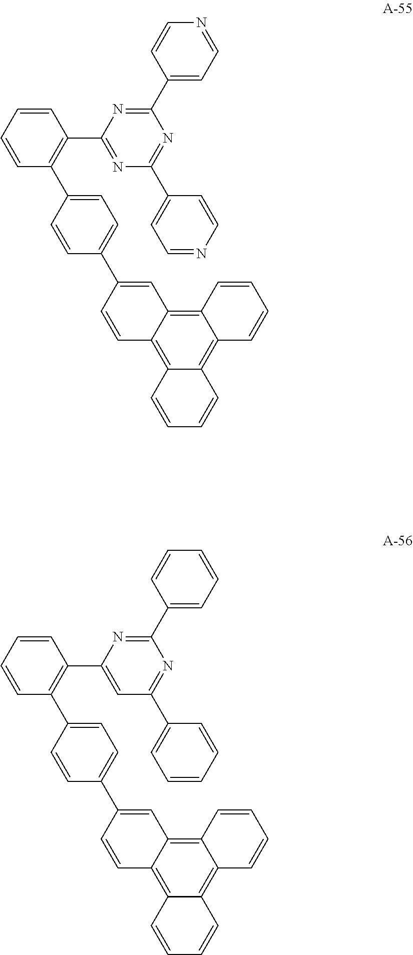 [SMILES: c1ccc(-c2nc(-c3ccncc3)nc(-c3ccncc3)n2)c(-c2ccc(-c3ccc4c5ccccc5c5ccccc5c4c3)cc2)c1 ; c1ccc(-c2cc(-c3ccccc3-c3ccc(-c4ccc5c6ccccc6c6ccccc6c5c4)cc3)nc(-c3ccccc3)n2)cc1]

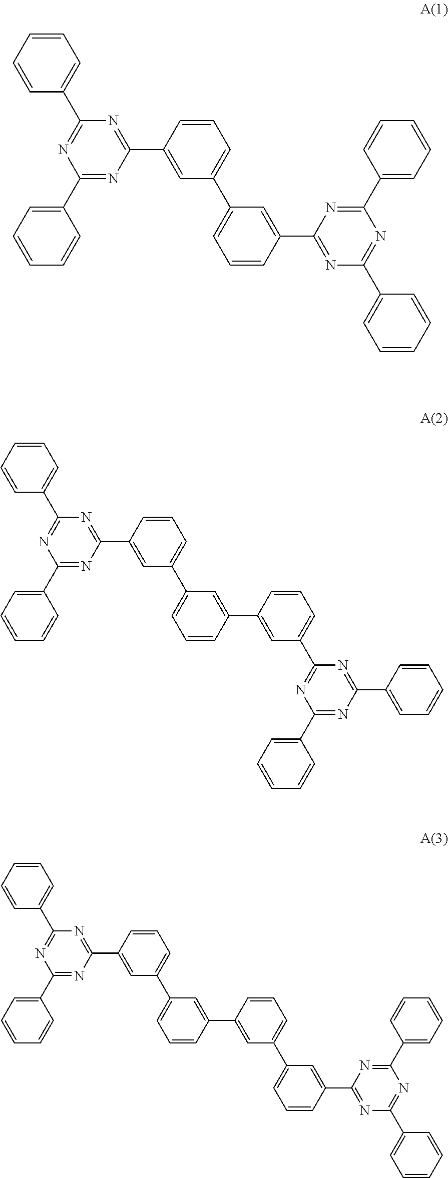 [SMILES: c1ccc(-c2nc(-c3ccccc3)nc(-c3cccc(-c4cccc(-c5nc(-c6ccccc6)nc(-c6ccccc6)n5)c4)c3)n2)cc1 ; c1ccc(-c2nc(-c3ccccc3)nc(-c3cccc(-c4cccc(-c5cccc(-c6nc(-c7ccccc7)nc(-c7ccccc7)n6)c5)c4)c3)n2)cc1 ; c1ccc(-c2nc(-c3ccccc3)nc(-c3cccc(-c4cccc(-c5cccc(-c6cccc(-c7nc(-c8ccccc8)nc(-c8ccccc8)n7)c6)c5)c4)c3)n2)cc1]

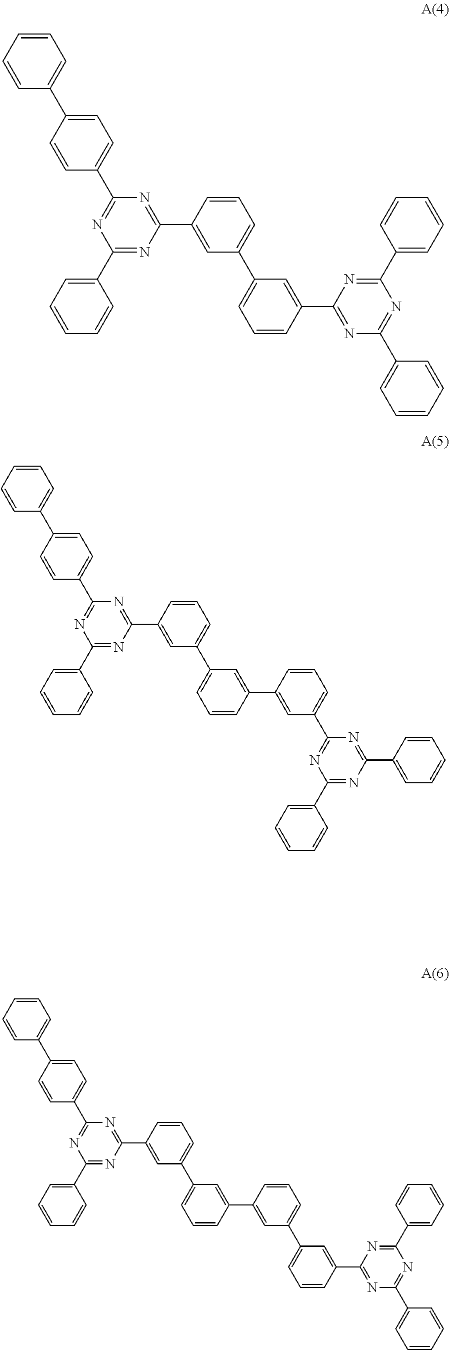 [SMILES: c1ccc(-c2ccc(-c3nc(-c4ccccc4)nc(-c4cccc(-c5cccc(-c6nc(-c7ccccc7)nc(-c7ccccc7)n6)c5)c4)n3)cc2)cc1 ; c1ccc(-c2ccc(-c3nc(-c4ccccc4)nc(-c4cccc(-c5cccc(-c6cccc(-c7nc(-c8ccccc8)nc(-c8ccccc8)n7)c6)c5)c4)n3)cc2)cc1 ; c1ccc(-c2ccc(-c3nc(-c4ccccc4)nc(-c4cccc(-c5cccc(-c6cccc(-c7cccc(-c8nc(-c9ccccc9)nc(-c9ccccc9)n8)c7)c6)c5)c4)n3)cc2)cc1]

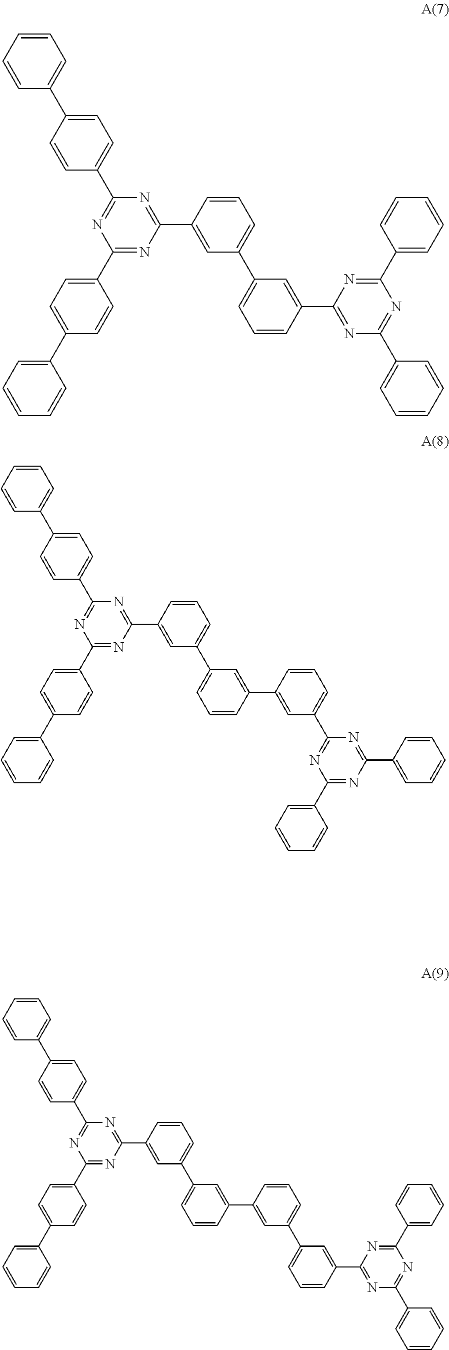 [SMILES: c1ccc(-c2ccc(-c3nc(-c4ccc(-c5ccccc5)cc4)nc(-c4cccc(-c5cccc(-c6nc(-c7ccccc7)nc(-c7ccccc7)n6)c5)c4)n3)cc2)cc1 ; c1ccc(-c2ccc(-c3nc(-c4ccc(-c5ccccc5)cc4)nc(-c4cccc(-c5cccc(-c6cccc(-c7nc(-c8ccccc8)nc(-c8ccccc8)n7)c6)c5)c4)n3)cc2)cc1 ; c1ccc(-c2ccc(-c3nc(-c4ccc(-c5ccccc5)cc4)nc(-c4cccc(-c5cccc(-c6cccc(-c7cccc(-c8nc(-c9ccccc9)nc(-c9ccccc9)n8)c7)c6)c5)c4)n3)cc2)cc1]

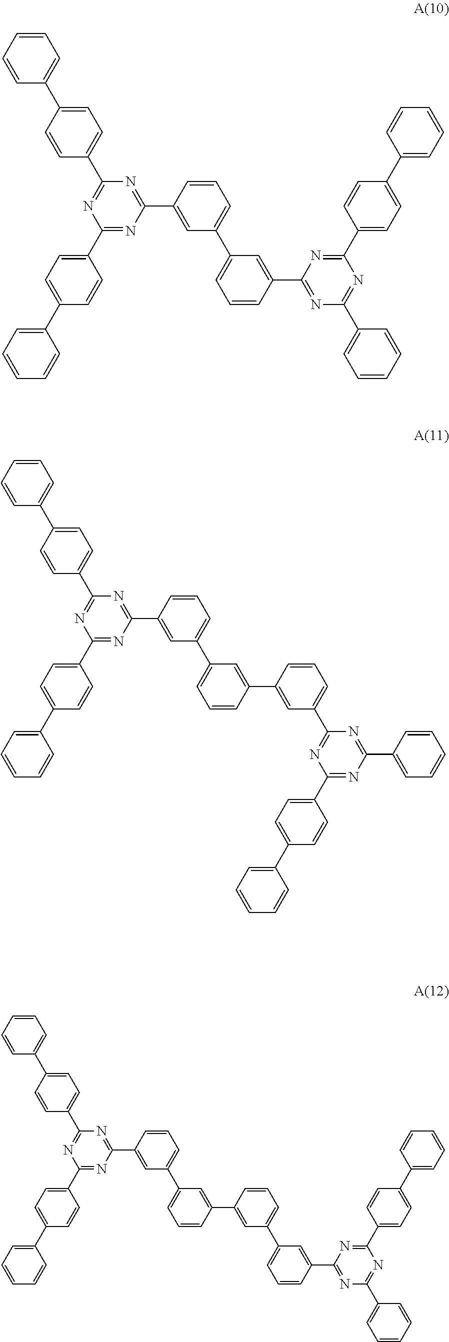 [SMILES: c1ccc(-c2ccc(-c3nc(-c4ccccc4)nc(-c4cccc(-c5cccc(-c6nc(-c7ccc(-c8ccccc8)cc7)nc(-c7ccc(-c8ccccc8)cc7)n6)c5)c4)n3)cc2)cc1 ; c1ccc(-c2ccc(-c3nc(-c4ccccc4)nc(-c4cccc(-c5cccc(-c6cccc(-c7nc(-c8ccc(-c9ccccc9)cc8)nc(-c8ccc(-c9ccccc9)cc8)n7)c6)c5)c4)n3)cc2)cc1 ; c1ccc(-c2ccc(-c3nc(-c4ccccc4)nc(-c4cccc(-c5cccc(-c6cccc(-c7cccc(-c8nc(-c9ccc(-c%10ccccc%10)cc9)nc(-c9ccc(-c%10ccccc%10)cc9)n8)c7)c6)c5)c4)n3)cc2)cc1]

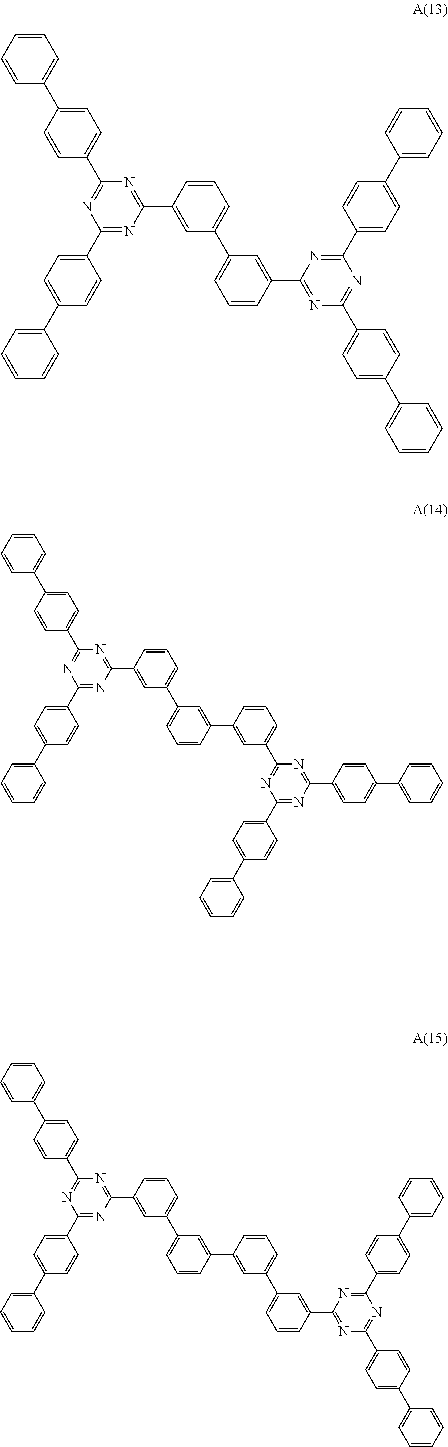 [SMILES: c1ccc(-c2ccc(-c3nc(-c4ccc(-c5ccccc5)cc4)nc(-c4cccc(-c5cccc(-c6nc(-c7ccc(-c8ccccc8)cc7)nc(-c7ccc(-c8ccccc8)cc7)n6)c5)c4)n3)cc2)cc1 ; c1ccc(-c2ccc(-c3nc(-c4ccc(-c5ccccc5)cc4)nc(-c4cccc(-c5cccc(-c6cccc(-c7nc(-c8ccc(-c9ccccc9)cc8)nc(-c8ccc(-c9ccccc9)cc8)n7)c6)c5)c4)n3)cc2)cc1 ; c1ccc(-c2ccc(-c3nc(-c4ccc(-c5ccccc5)cc4)nc(-c4cccc(-c5cccc(-c6cccc(-c7cccc(-c8nc(-c9ccc(-c%10ccccc%10)cc9)nc(-c9ccc(-c%10ccccc%10)cc9)n8)c7)c6)c5)c4)n3)cc2)cc1]

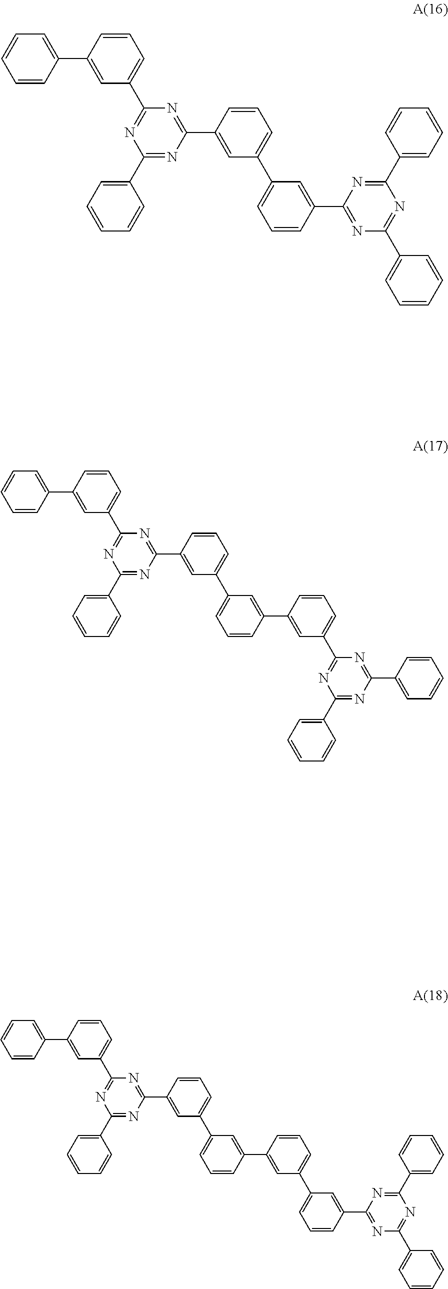 [SMILES: c1ccc(-c2cccc(-c3nc(-c4ccccc4)nc(-c4cccc(-c5cccc(-c6nc(-c7ccccc7)nc(-c7ccccc7)n6)c5)c4)n3)c2)cc1 ; c1ccc(-c2cccc(-c3nc(-c4ccccc4)nc(-c4cccc(-c5cccc(-c6cccc(-c7nc(-c8ccccc8)nc(-c8ccccc8)n7)c6)c5)c4)n3)c2)cc1 ; c1ccc(-c2cccc(-c3nc(-c4ccccc4)nc(-c4cccc(-c5cccc(-c6cccc(-c7cccc(-c8nc(-c9ccccc9)nc(-c9ccccc9)n8)c7)c6)c5)c4)n3)c2)cc1]

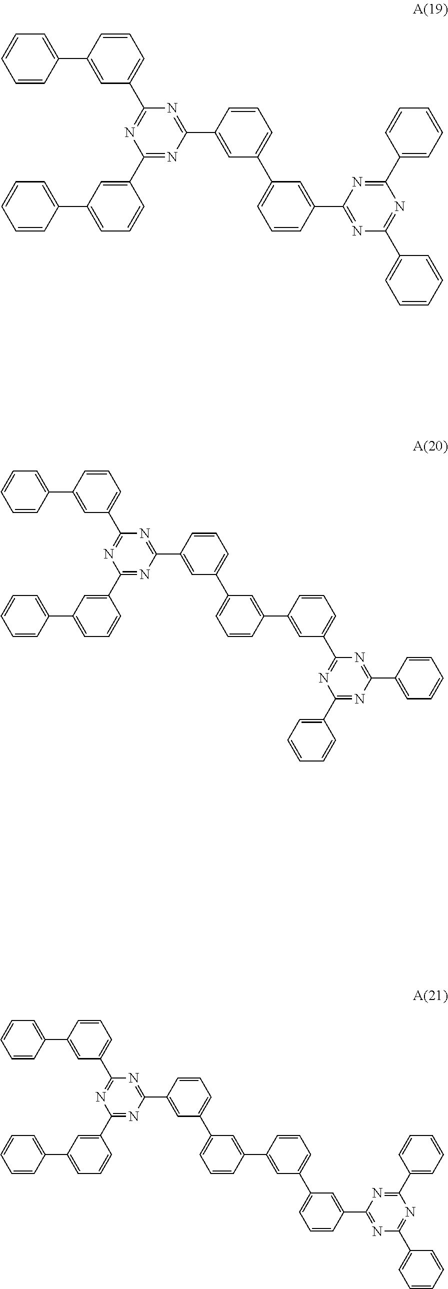 [SMILES: c1ccc(-c2cccc(-c3nc(-c4cccc(-c5ccccc5)c4)nc(-c4cccc(-c5cccc(-c6nc(-c7ccccc7)nc(-c7ccccc7)n6)c5)c4)n3)c2)cc1 ; c1ccc(-c2cccc(-c3nc(-c4cccc(-c5ccccc5)c4)nc(-c4cccc(-c5cccc(-c6cccc(-c7nc(-c8ccccc8)nc(-c8ccccc8)n7)c6)c5)c4)n3)c2)cc1 ; c1ccc(-c2cccc(-c3nc(-c4cccc(-c5ccccc5)c4)nc(-c4cccc(-c5cccc(-c6cccc(-c7cccc(-c8nc(-c9ccccc9)nc(-c9ccccc9)n8)c7)c6)c5)c4)n3)c2)cc1]

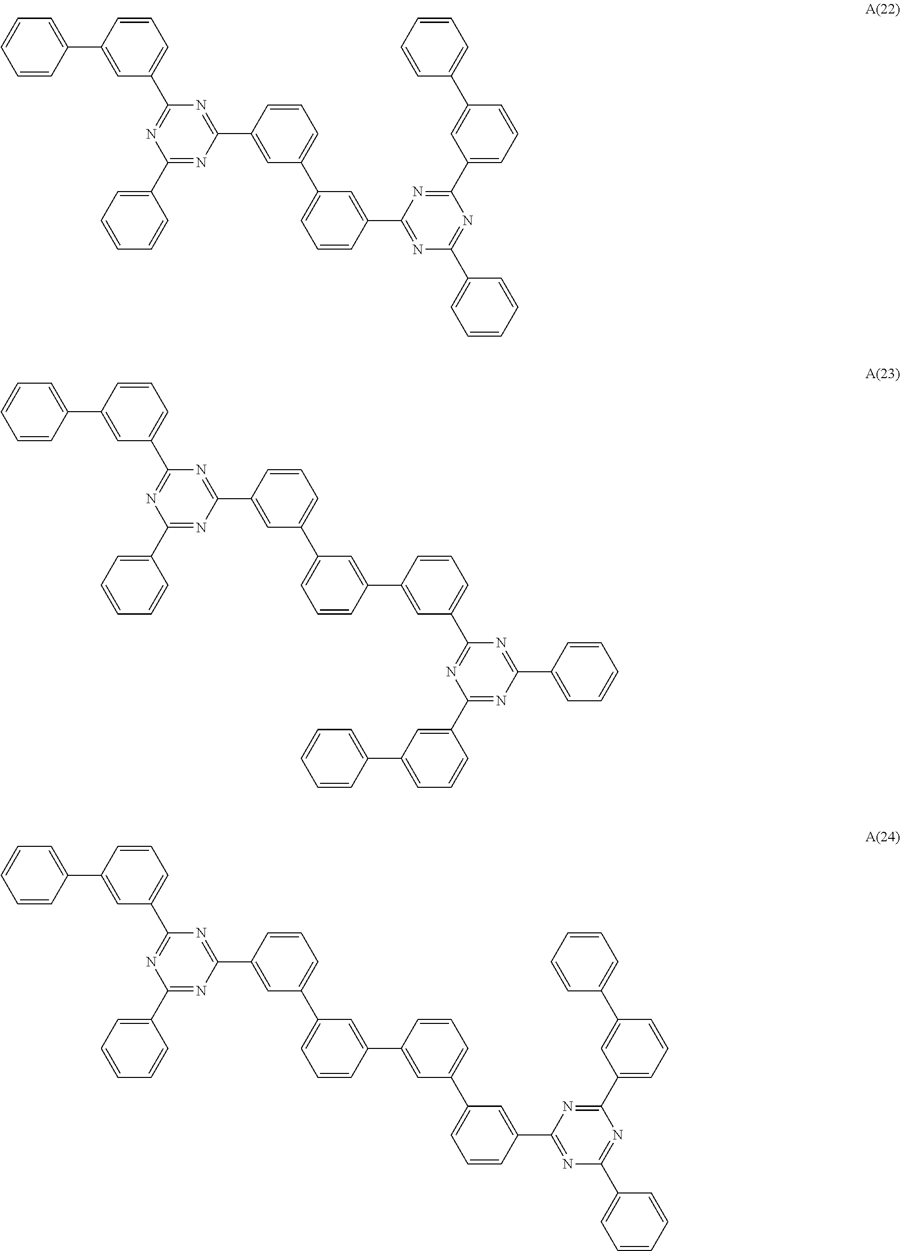 [SMILES: c1ccc(-c2cccc(-c3nc(-c4ccccc4)nc(-c4cccc(-c5cccc(-c6nc(-c7ccccc7)nc(-c7cccc(-c8ccccc8)c7)n6)c5)c4)n3)c2)cc1 ; c1ccc(-c2cccc(-c3nc(-c4ccccc4)nc(-c4cccc(-c5cccc(-c6cccc(-c7nc(-c8ccccc8)nc(-c8cccc(-c9ccccc9)c8)n7)c6)c5)c4)n3)c2)cc1 ; c1ccc(-c2cccc(-c3nc(-c4ccccc4)nc(-c4cccc(-c5cccc(-c6cccc(-c7cccc(-c8nc(-c9ccccc9)nc(-c9cccc(-c%10ccccc%10)c9)n8)c7)c6)c5)c4)n3)c2)cc1]

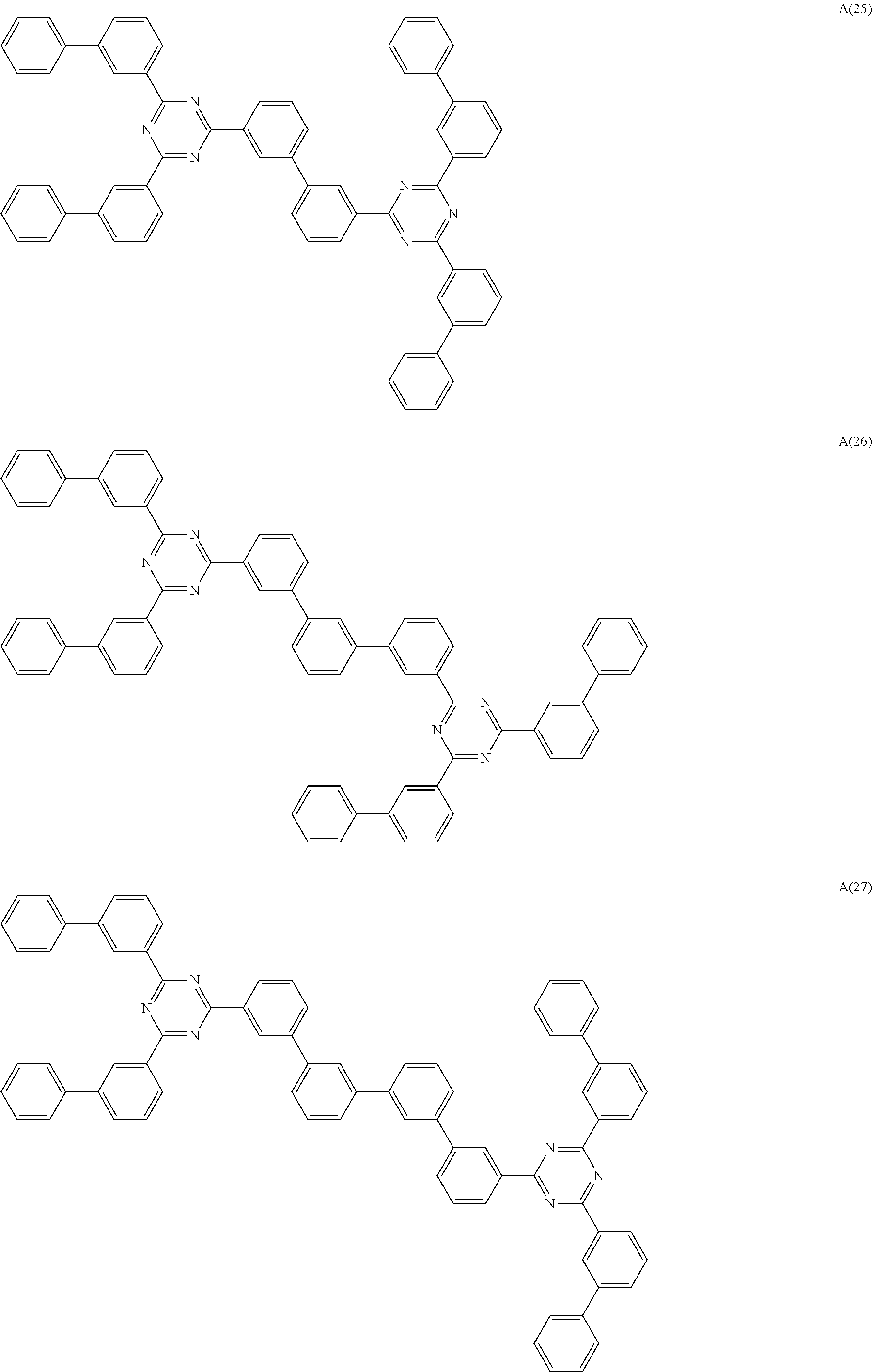 [SMILES: c1ccc(-c2cccc(-c3nc(-c4cccc(-c5ccccc5)c4)nc(-c4cccc(-c5cccc(-c6nc(-c7cccc(-c8ccccc8)c7)nc(-c7cccc(-c8ccccc8)c7)n6)c5)c4)n3)c2)cc1 ; c1ccc(-c2cccc(-c3nc(-c4cccc(-c5ccccc5)c4)nc(-c4cccc(-c5cccc(-c6cccc(-c7nc(-c8cccc(-c9ccccc9)c8)nc(-c8cccc(-c9ccccc9)c8)n7)c6)c5)c4)n3)c2)cc1 ; c1ccc(-c2cccc(-c3nc(-c4cccc(-c5ccccc5)c4)nc(-c4cccc(-c5cccc(-c6cccc(-c7cccc(-c8nc(-c9cccc(-c%10ccccc%10)c9)nc(-c9cccc(-c%10ccccc%10)c9)n8)c7)c6)c5)c4)n3)c2)cc1]

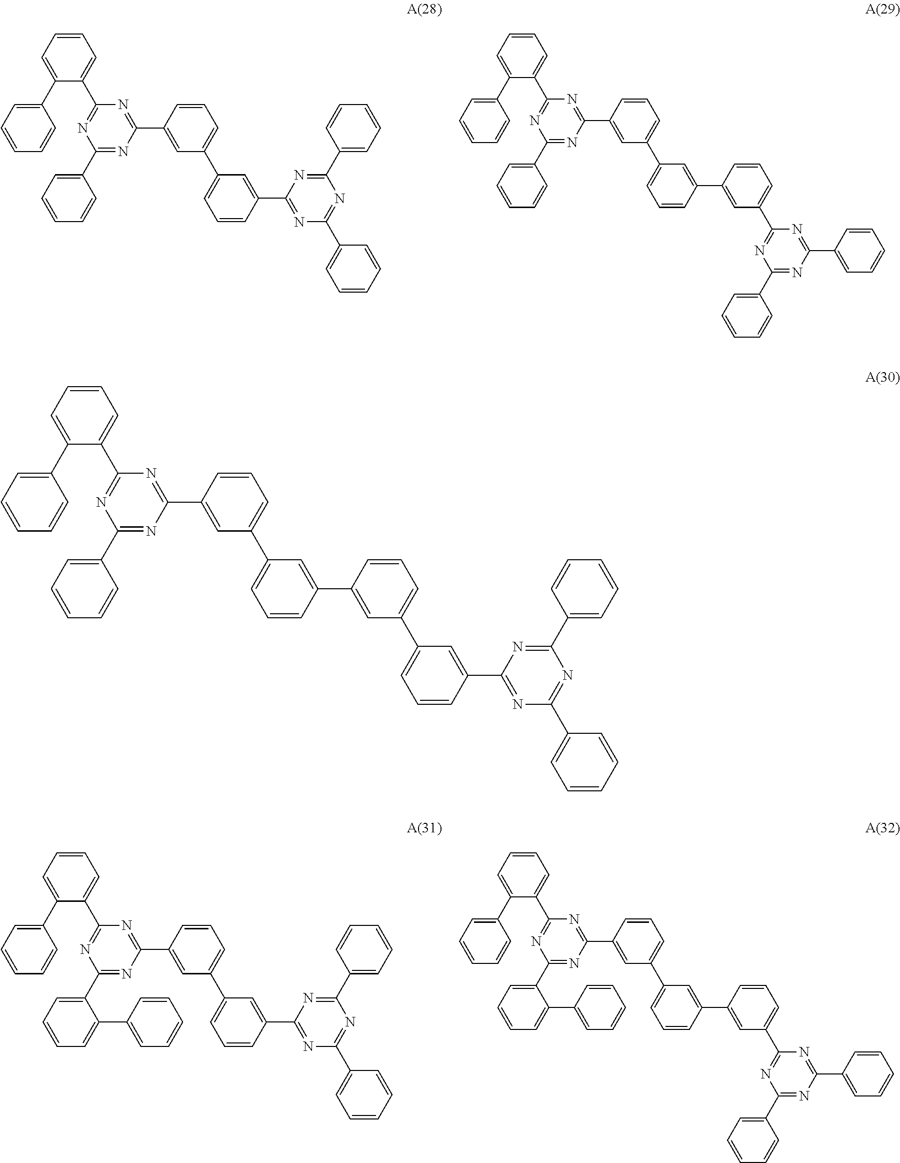

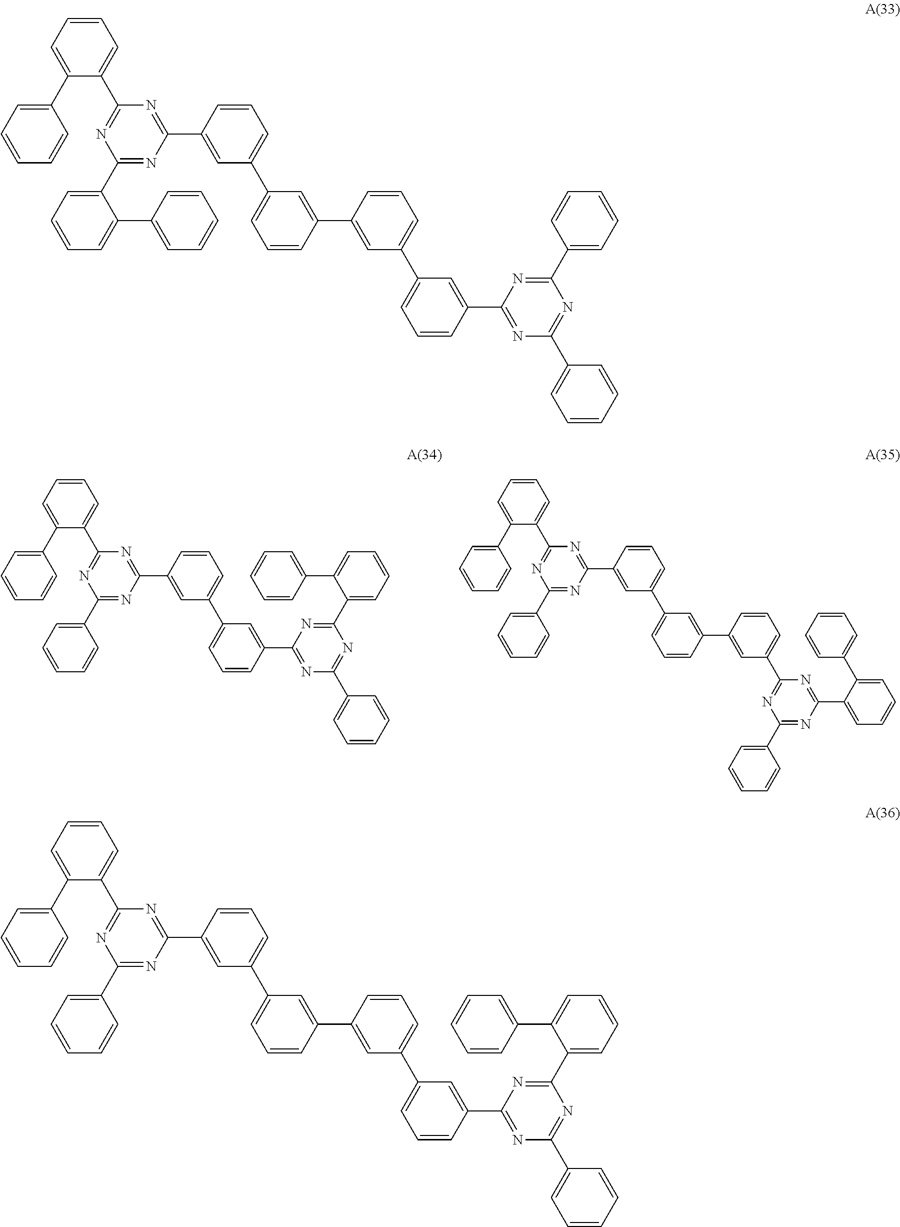 [SMILES: c1ccc(-c2nc(-c3ccccc3)nc(-c3cccc(-c4cccc(-c5cccc(-c6cccc(-c7nc(-c8ccccc8-c8ccccc8)nc(-c8ccccc8-c8ccccc8)n7)c6)c5)c4)c3)n2)cc1 ; c1ccc(-c2nc(-c3cccc(-c4cccc(-c5nc(-c6ccccc6)nc(-c6ccccc6-c6ccccc6)n5)c4)c3)nc(-c3ccccc3-c3ccccc3)n2)cc1 ; c1ccc(-c2nc(-c3cccc(-c4cccc(-c5cccc(-c6nc(-c7ccccc7)nc(-c7ccccc7-c7ccccc7)n6)c5)c4)c3)nc(-c3ccccc3-c3ccccc3)n2)cc1 ; c1ccc(-c2nc(-c3cccc(-c4cccc(-c5cccc(-c6cccc(-c7nc(-c8ccccc8)nc(-c8ccccc8-c8ccccc8)n7)c6)c5)c4)c3)nc(-c3ccccc3-c3ccccc3)n2)cc1]

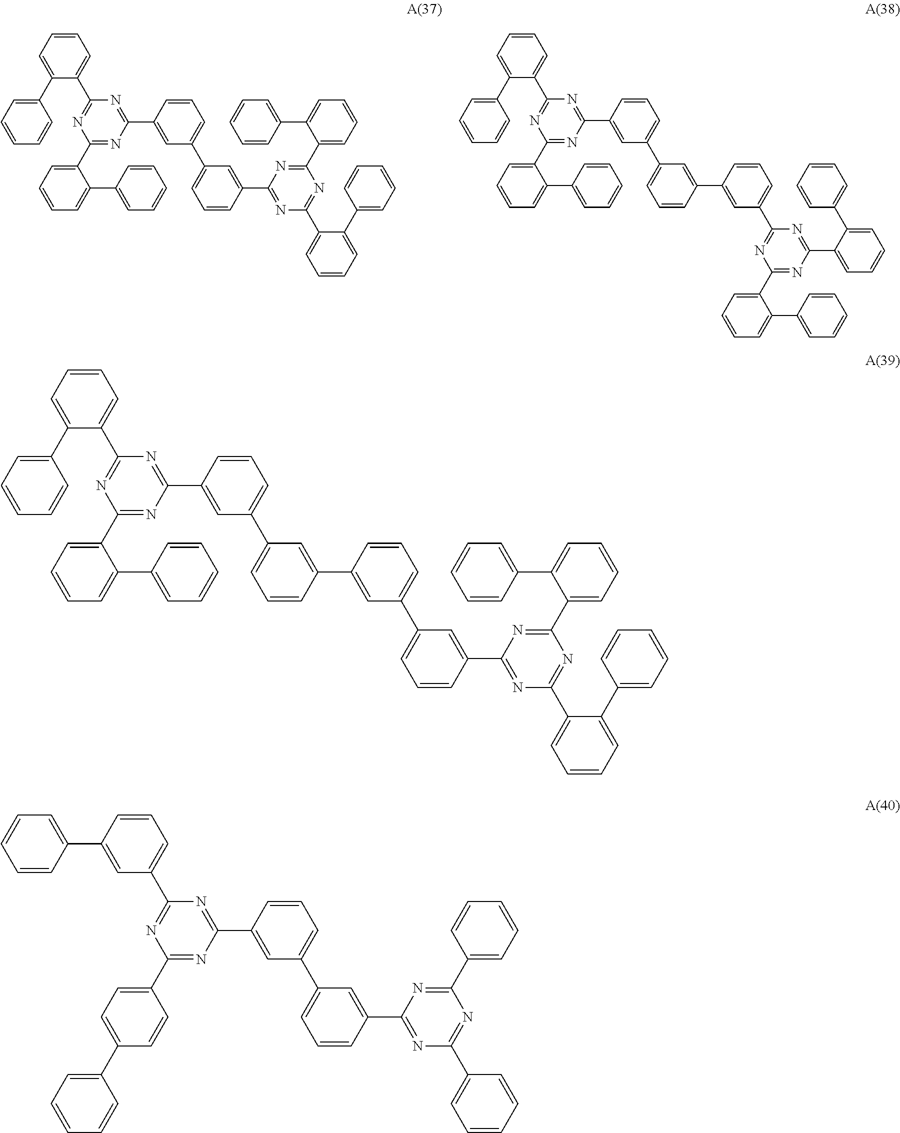

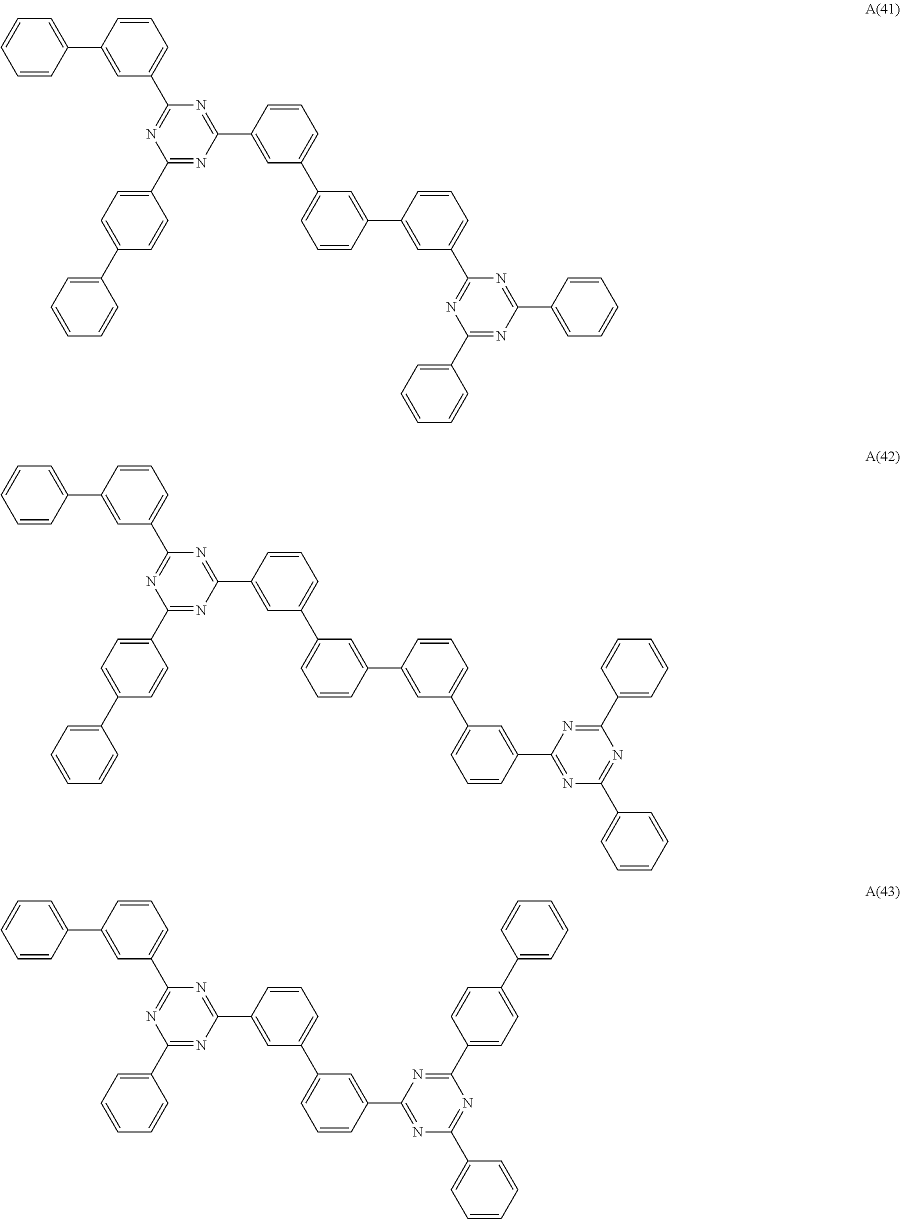 [SMILES: c1ccc(-c2ccc(-c3nc(-c4cccc(-c5ccccc5)c4)nc(-c4cccc(-c5cccc(-c6cccc(-c7nc(-c8ccccc8)nc(-c8ccccc8)n7)c6)c5)c4)n3)cc2)cc1 ; c1ccc(-c2ccc(-c3nc(-c4cccc(-c5ccccc5)c4)nc(-c4cccc(-c5cccc(-c6cccc(-c7cccc(-c8nc(-c9ccccc9)nc(-c9ccccc9)n8)c7)c6)c5)c4)n3)cc2)cc1 ; c1ccc(-c2ccc(-c3nc(-c4ccccc4)nc(-c4cccc(-c5cccc(-c6nc(-c7ccccc7)nc(-c7cccc(-c8ccccc8)c7)n6)c5)c4)n3)cc2)cc1]

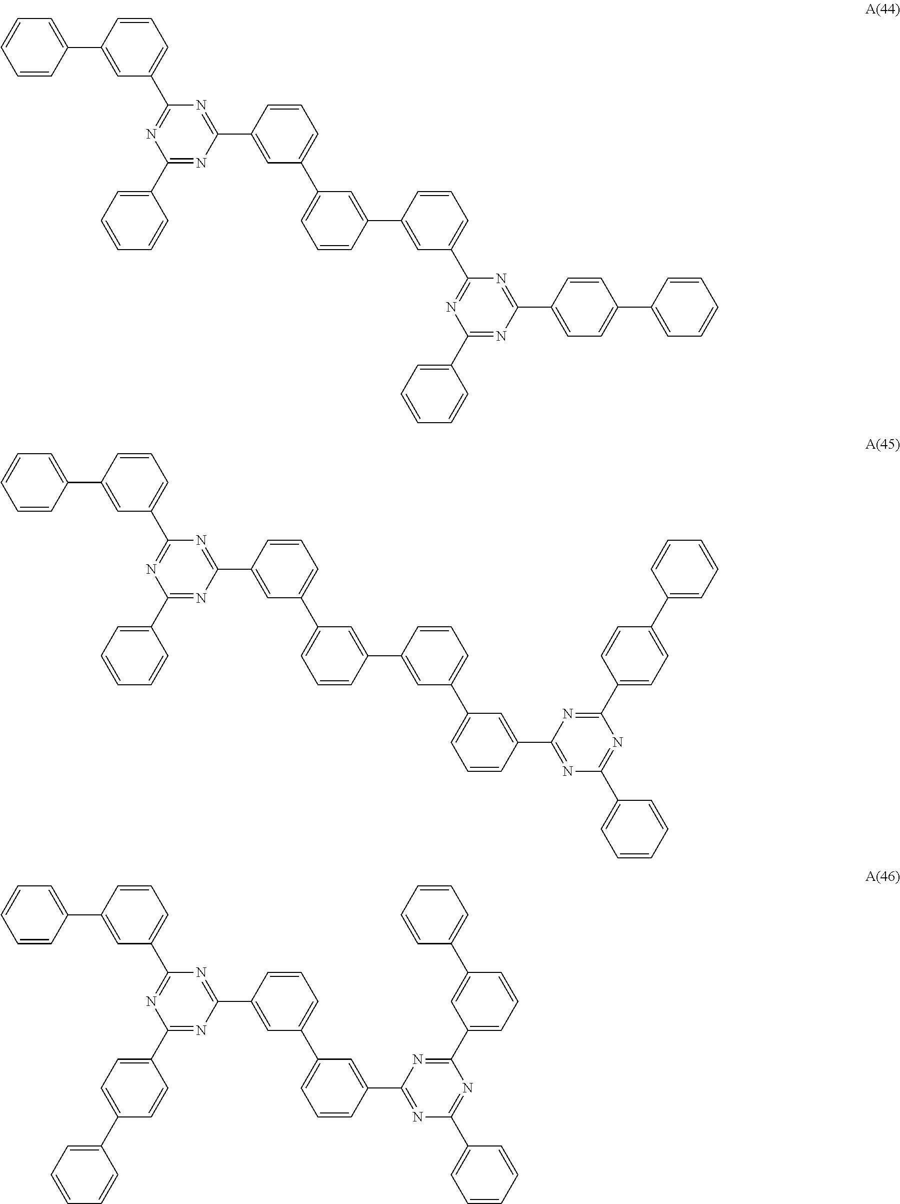 [SMILES: c1ccc(-c2ccc(-c3nc(-c4ccccc4)nc(-c4cccc(-c5cccc(-c6cccc(-c7nc(-c8ccccc8)nc(-c8cccc(-c9ccccc9)c8)n7)c6)c5)c4)n3)cc2)cc1 ; c1ccc(-c2ccc(-c3nc(-c4ccccc4)nc(-c4cccc(-c5cccc(-c6cccc(-c7cccc(-c8nc(-c9ccccc9)nc(-c9cccc(-c%10ccccc%10)c9)n8)c7)c6)c5)c4)n3)cc2)cc1 ; c1ccc(-c2ccc(-c3nc(-c4cccc(-c5ccccc5)c4)nc(-c4cccc(-c5cccc(-c6nc(-c7ccccc7)nc(-c7cccc(-c8ccccc8)c7)n6)c5)c4)n3)cc2)cc1]

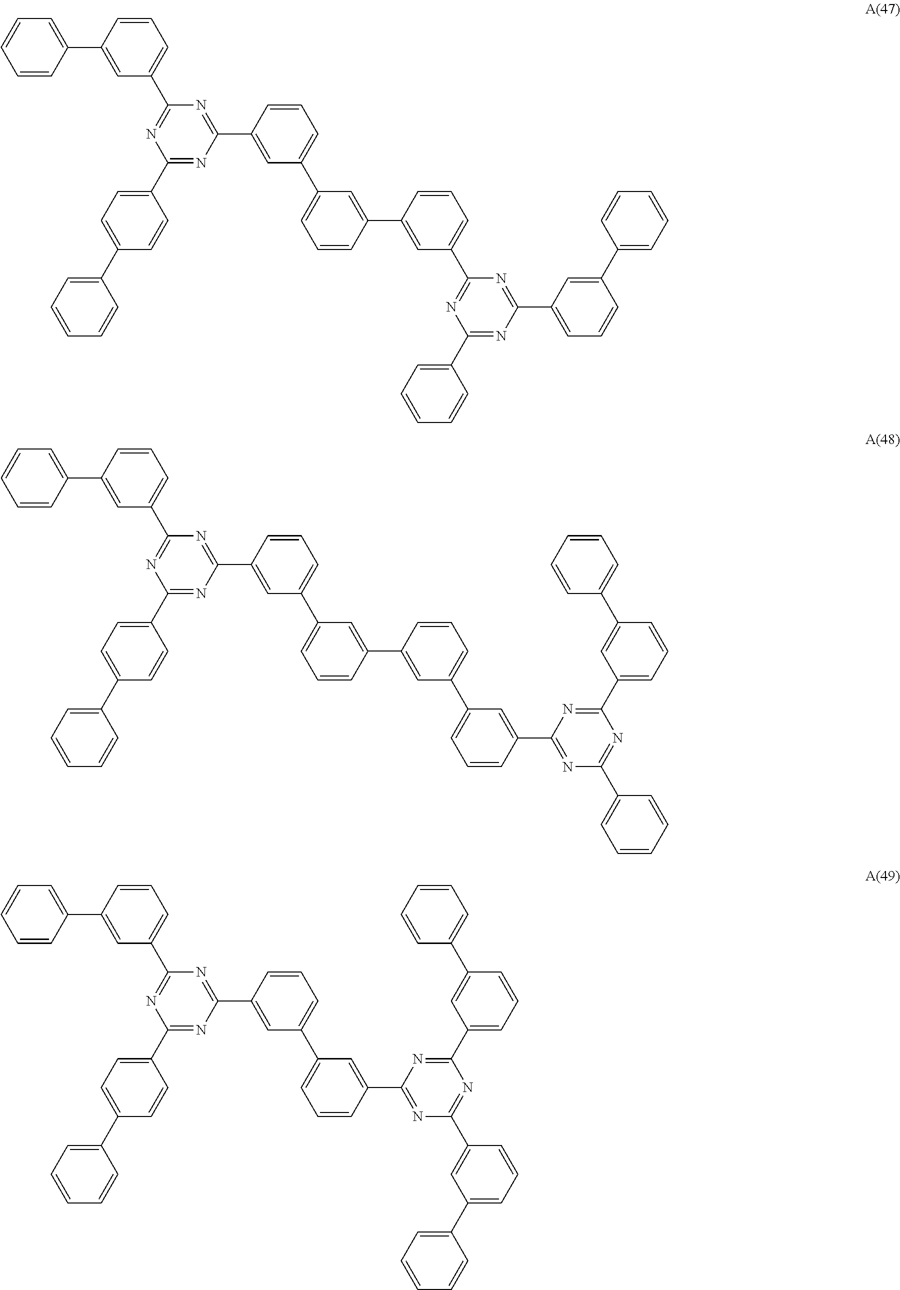 [SMILES: c1ccc(-c2ccc(-c3nc(-c4cccc(-c5ccccc5)c4)nc(-c4cccc(-c5cccc(-c6cccc(-c7nc(-c8ccccc8)nc(-c8cccc(-c9ccccc9)c8)n7)c6)c5)c4)n3)cc2)cc1 ; c1ccc(-c2ccc(-c3nc(-c4cccc(-c5ccccc5)c4)nc(-c4cccc(-c5cccc(-c6cccc(-c7cccc(-c8nc(-c9ccccc9)nc(-c9cccc(-c%10ccccc%10)c9)n8)c7)c6)c5)c4)n3)cc2)cc1 ; c1ccc(-c2ccc(-c3nc(-c4cccc(-c5ccccc5)c4)nc(-c4cccc(-c5cccc(-c6nc(-c7cccc(-c8ccccc8)c7)nc(-c7cccc(-c8ccccc8)c7)n6)c5)c4)n3)cc2)cc1]

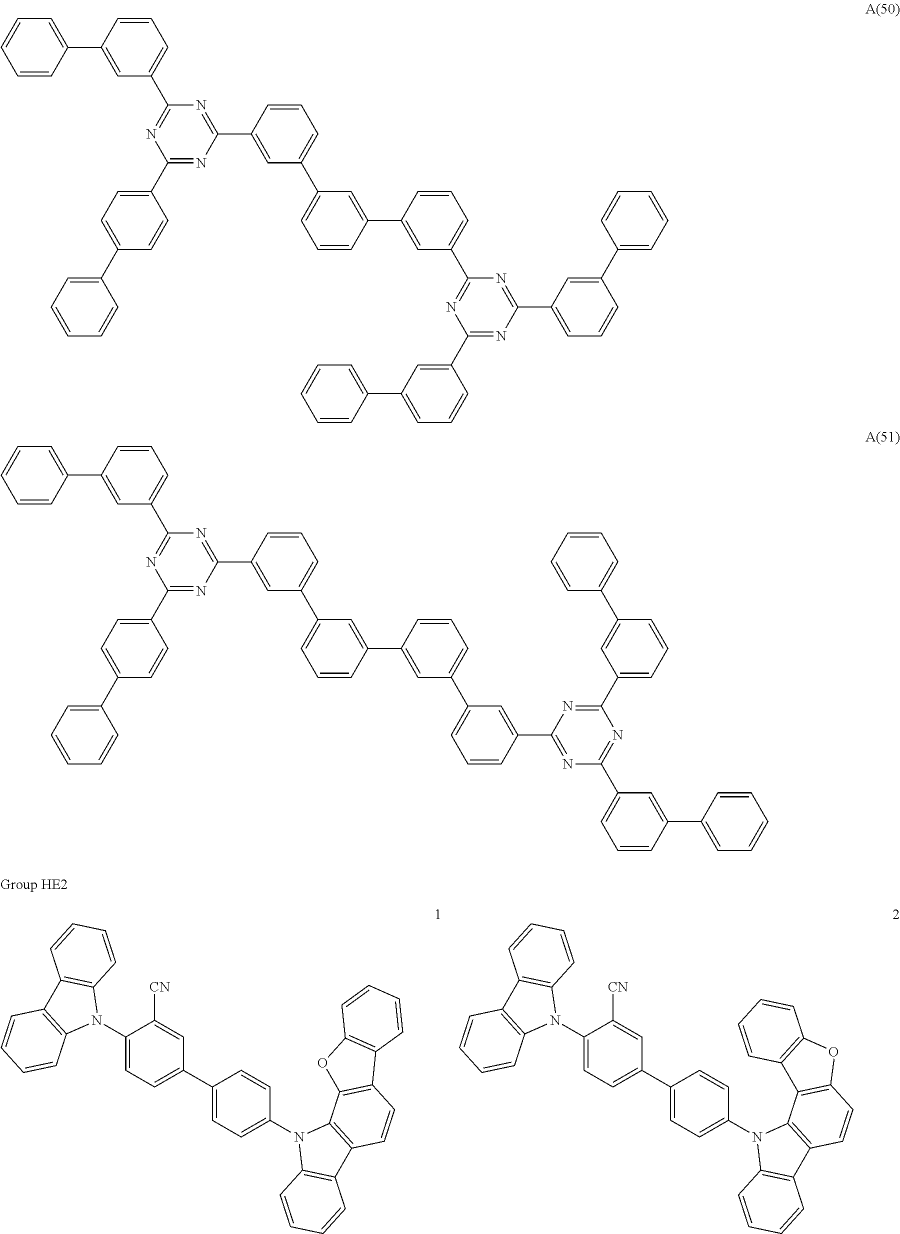 [SMILES: c1ccc(-c2ccc(-c3nc(-c4cccc(-c5ccccc5)c4)nc(-c4cccc(-c5cccc(-c6cccc(-c7nc(-c8cccc(-c9ccccc9)c8)nc(-c8cccc(-c9ccccc9)c8)n7)c6)c5)c4)n3)cc2)cc1 ; c1ccc(-c2ccc(-c3nc(-c4cccc(-c5ccccc5)c4)nc(-c4cccc(-c5cccc(-c6cccc(-c7cccc(-c8nc(-c9cccc(-c%10ccccc%10)c9)nc(-c9cccc(-c%10ccccc%10)c9)n8)c7)c6)c5)c4)n3)cc2)cc1 ; N#Cc1cc(-c2ccc(-n3c4ccccc4c4ccc5c6ccccc6oc5c43)cc2)ccc1-n1c2ccccc2c2ccccc21 ; N#Cc1cc(-c2ccc(-n3c4ccccc4c4ccc5oc6ccccc6c5c43)cc2)ccc1-n1c2ccccc2c2ccccc21]

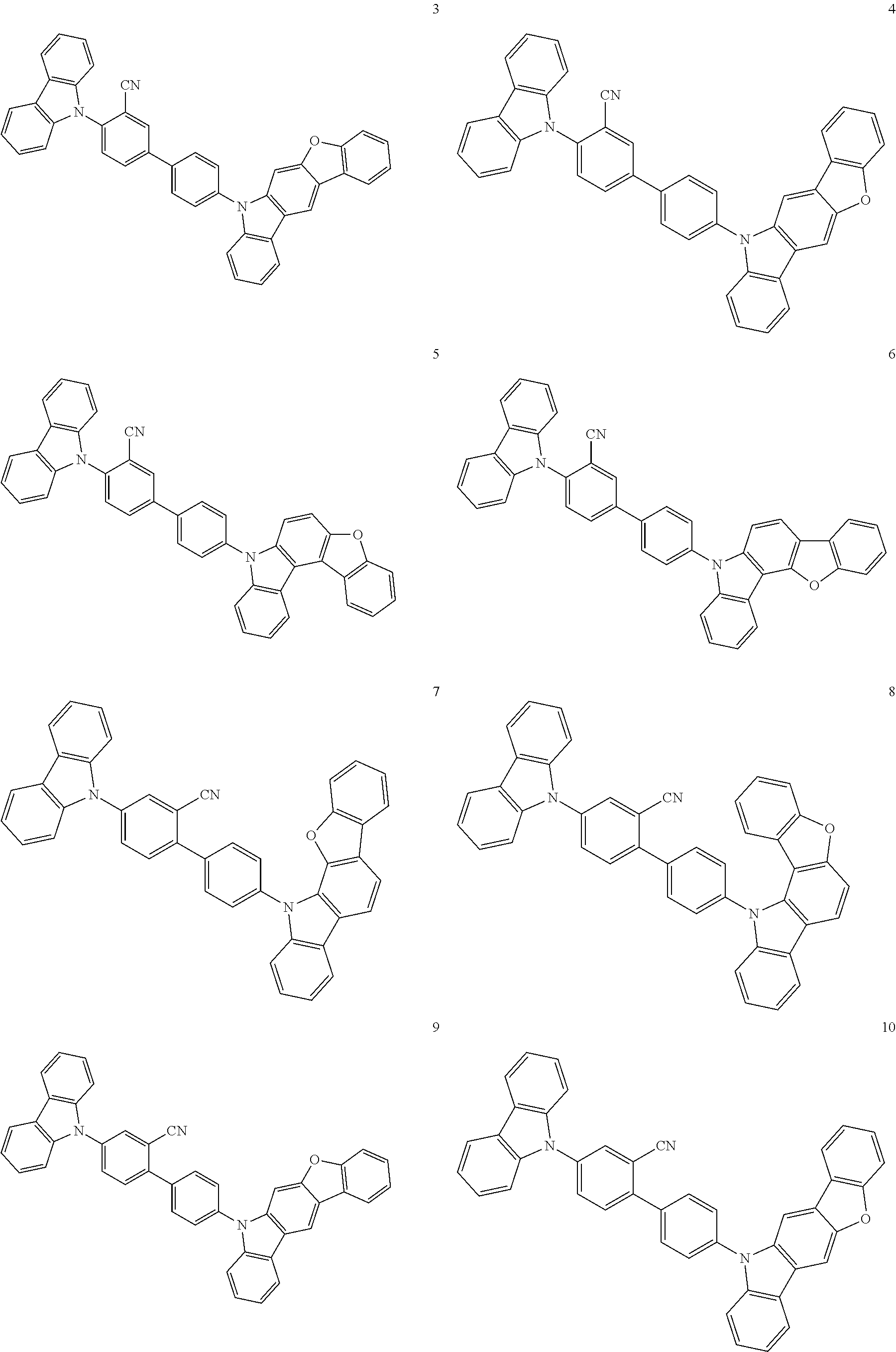

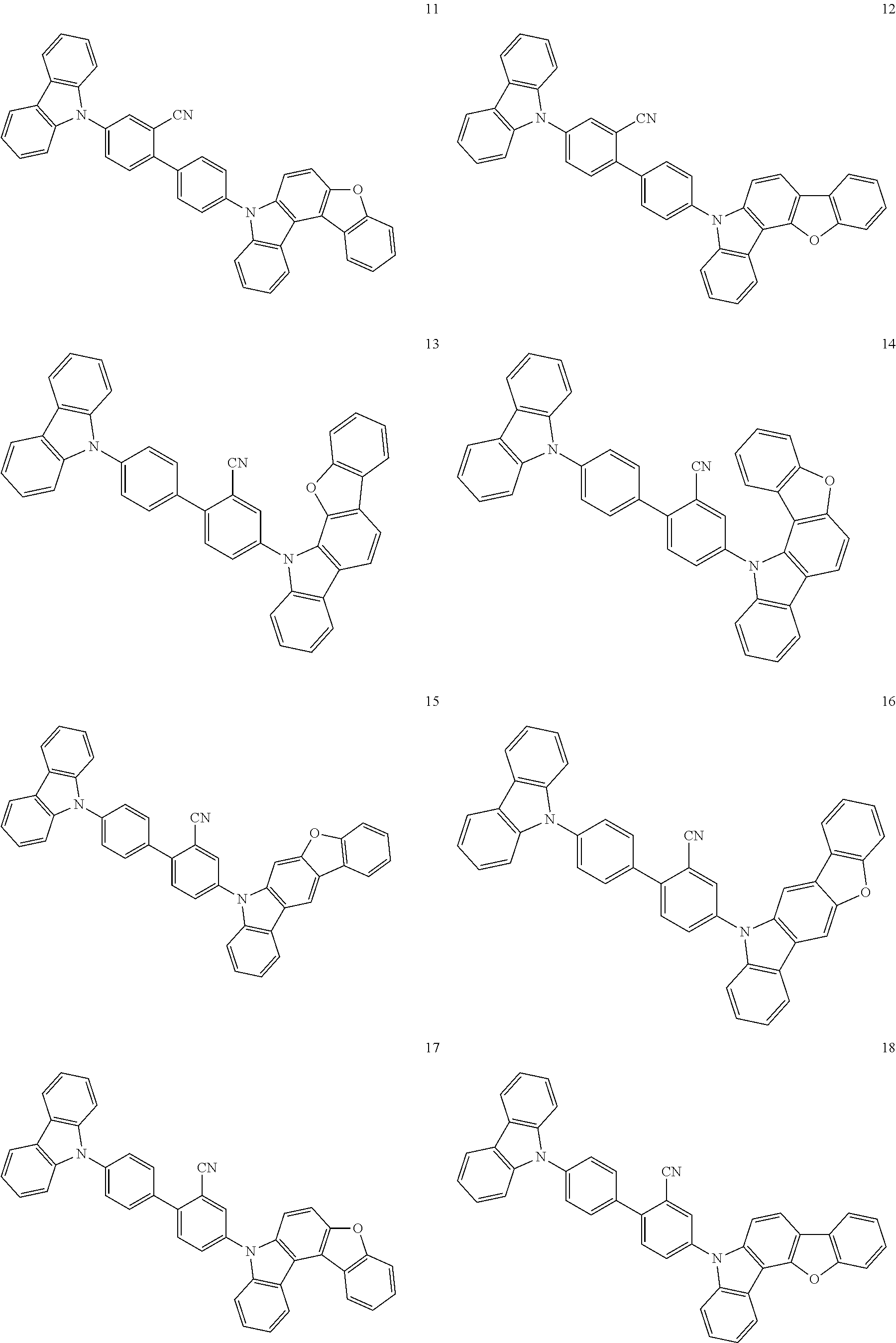

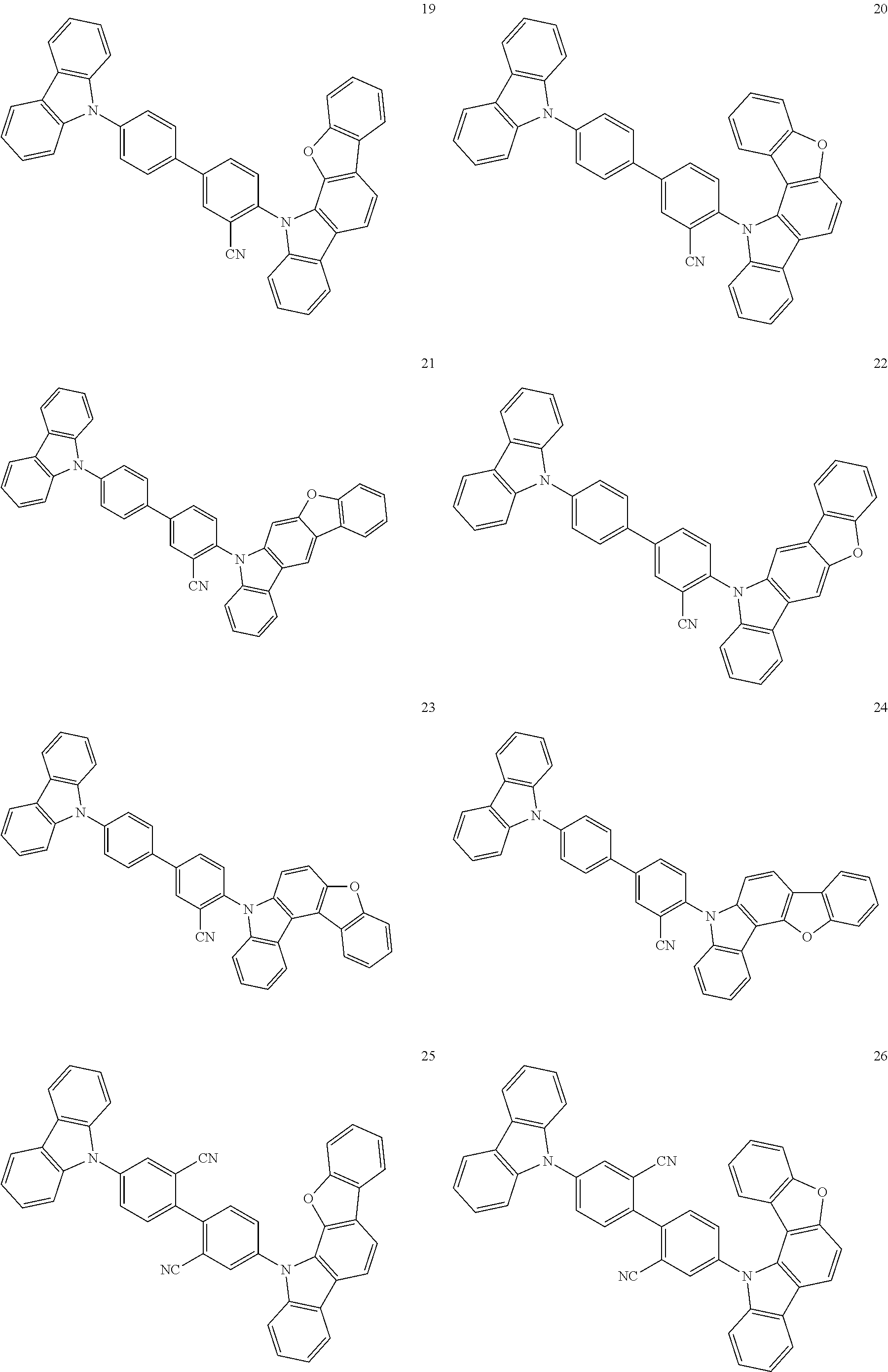

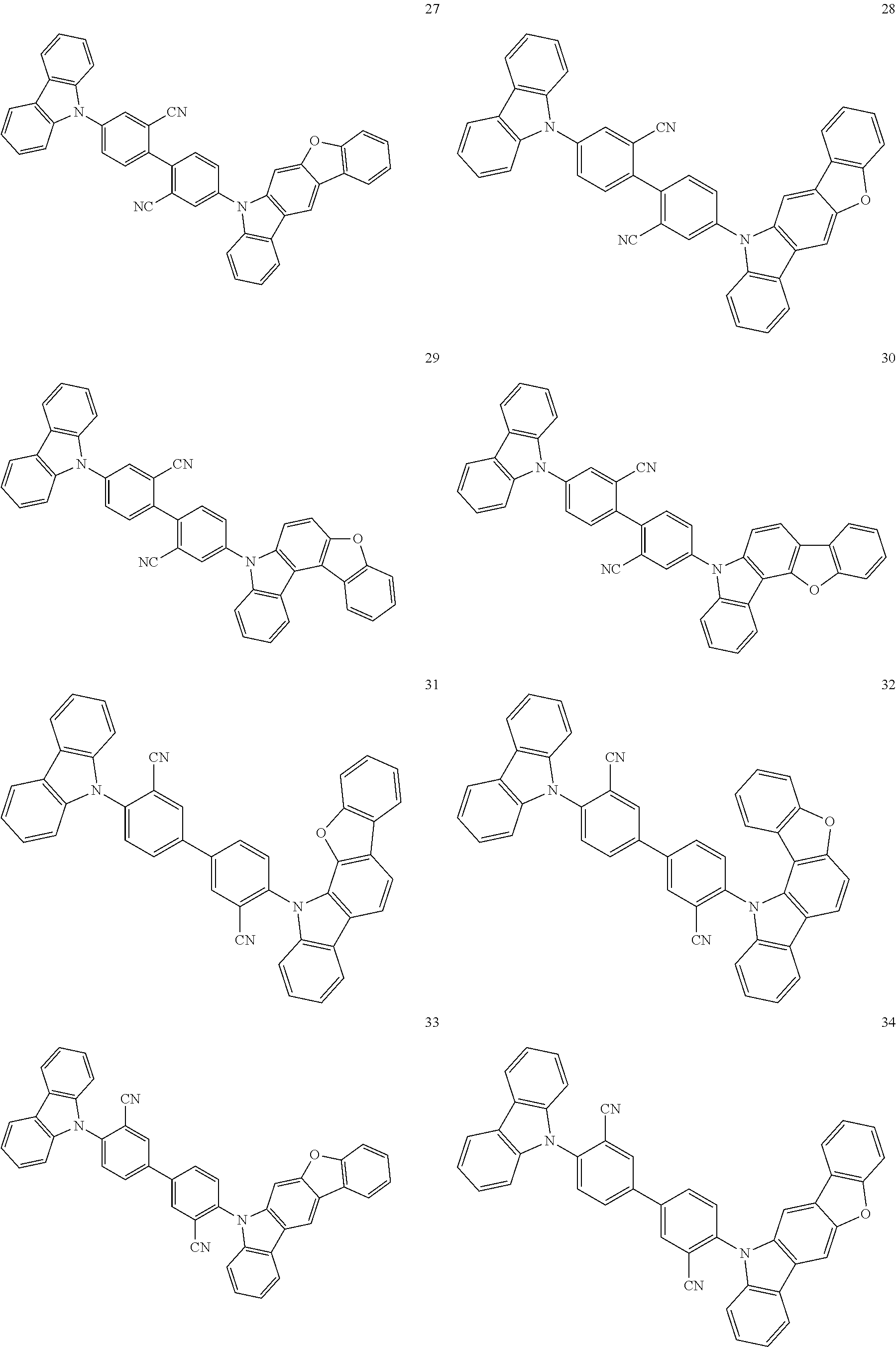

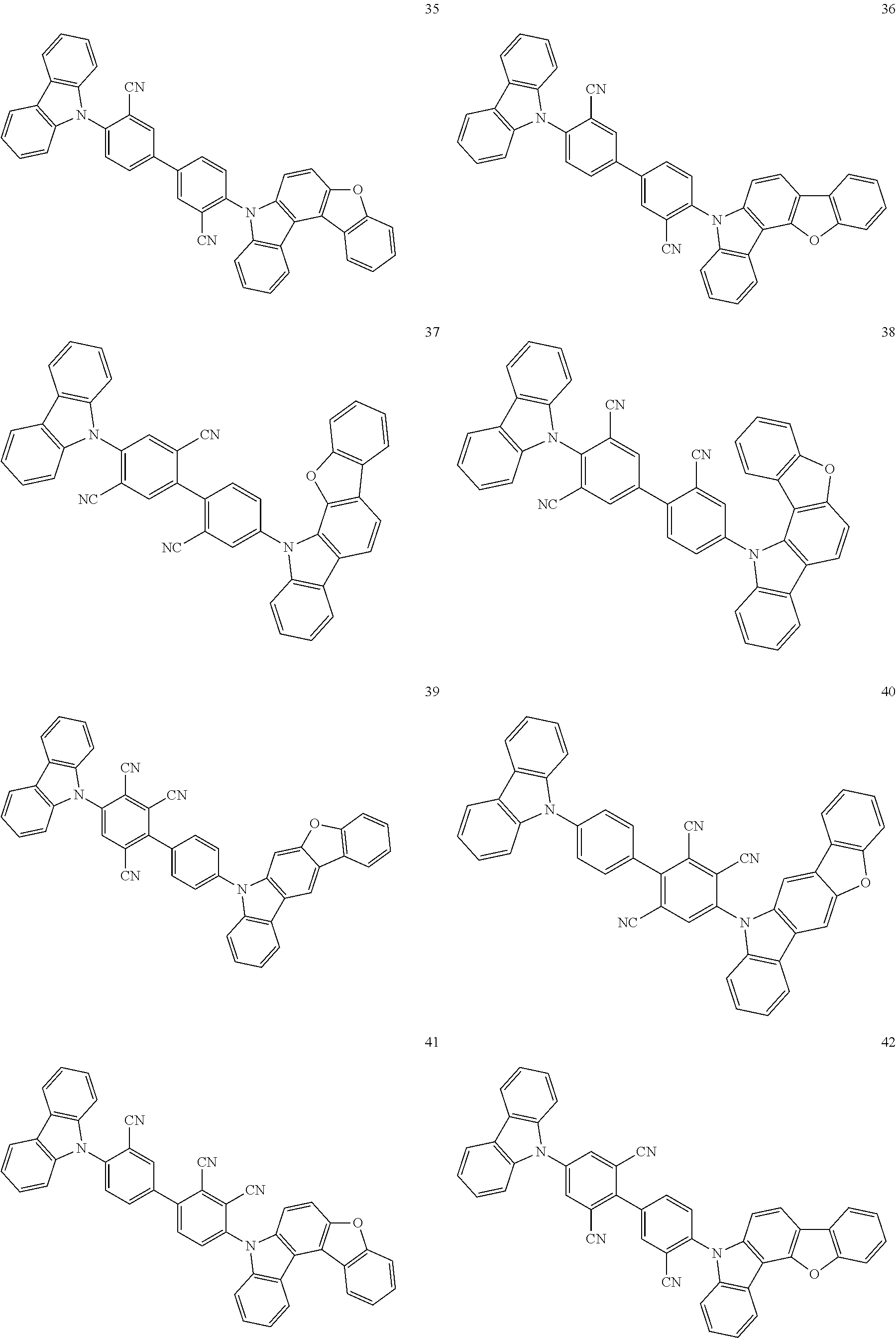

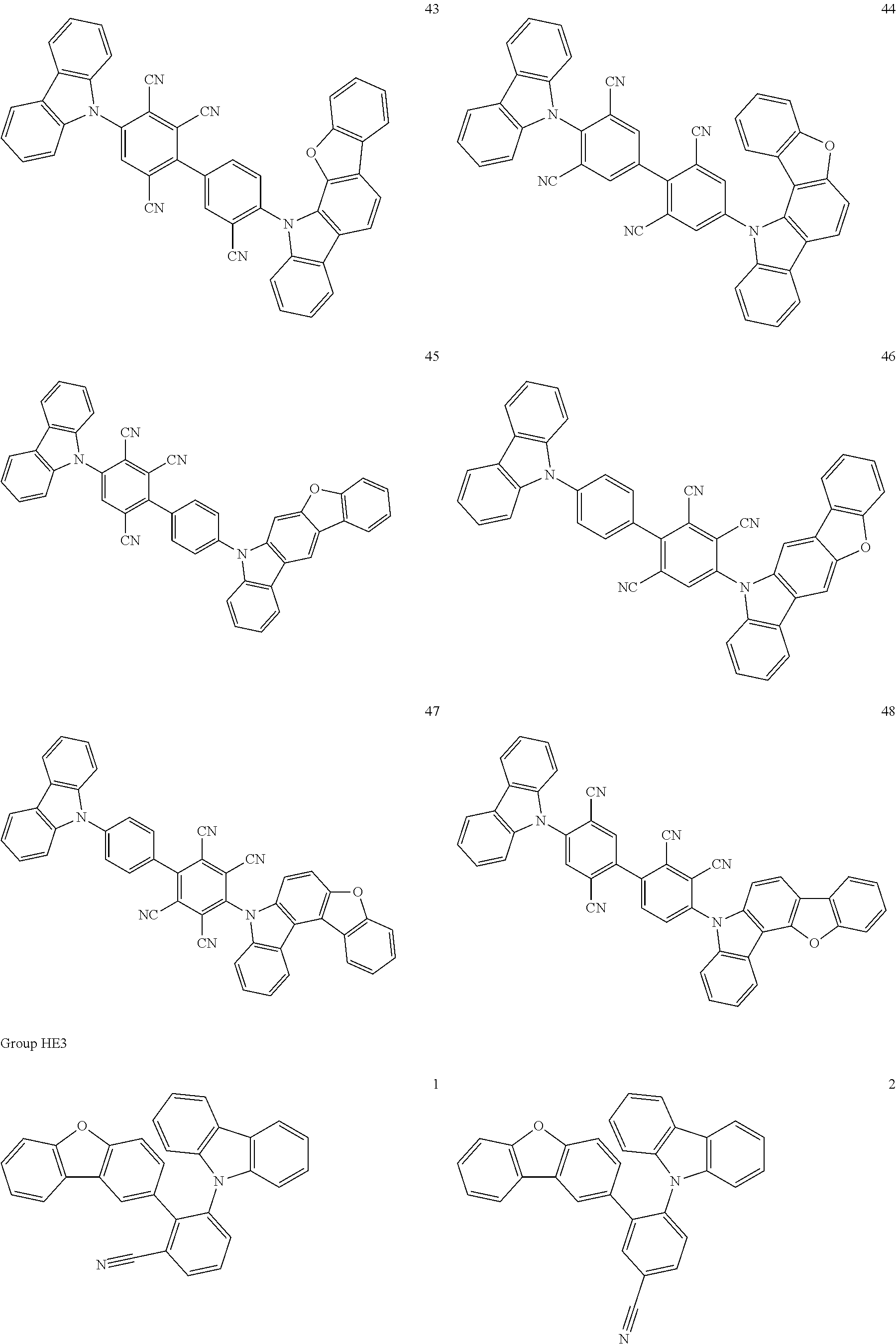

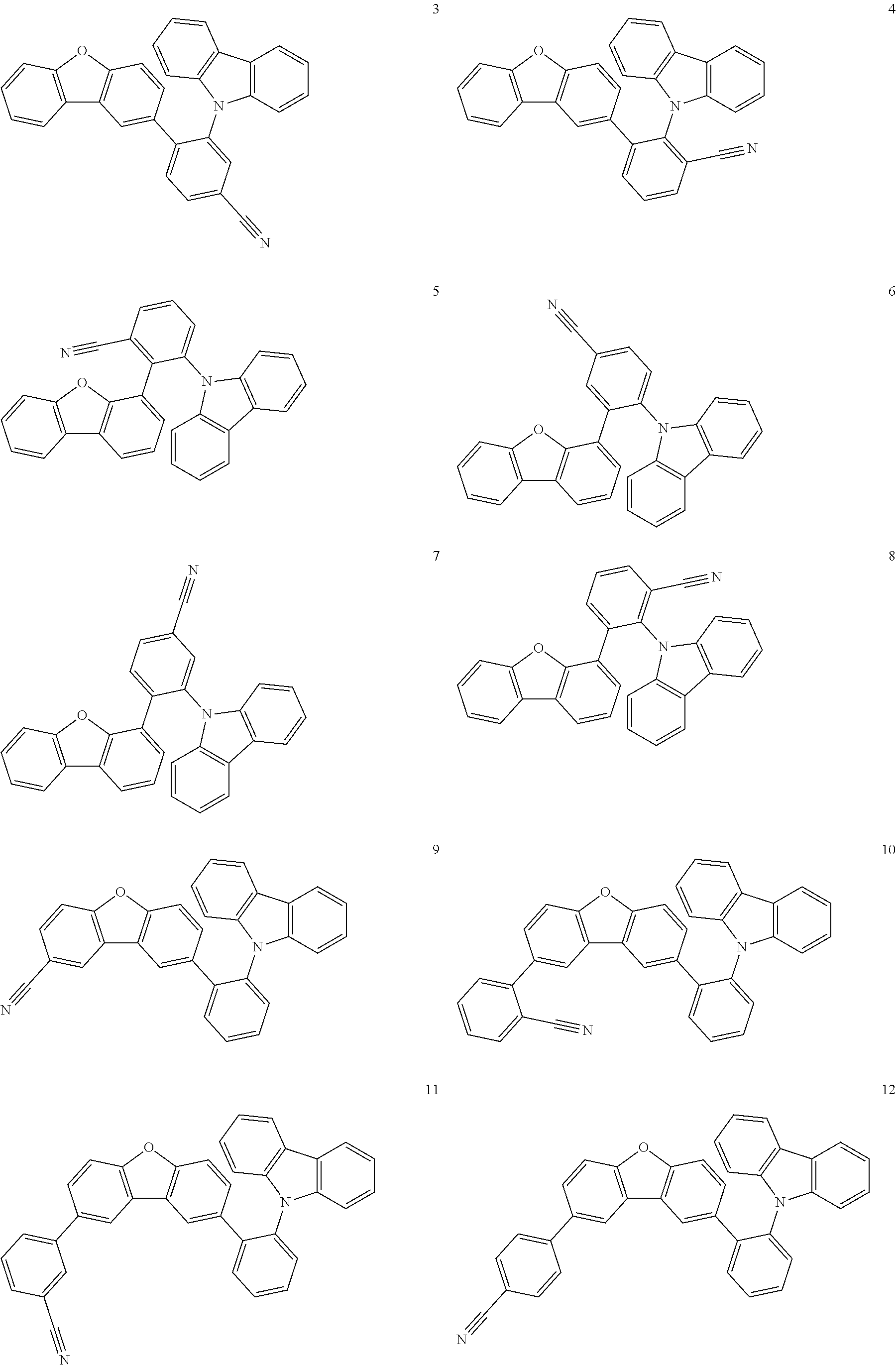

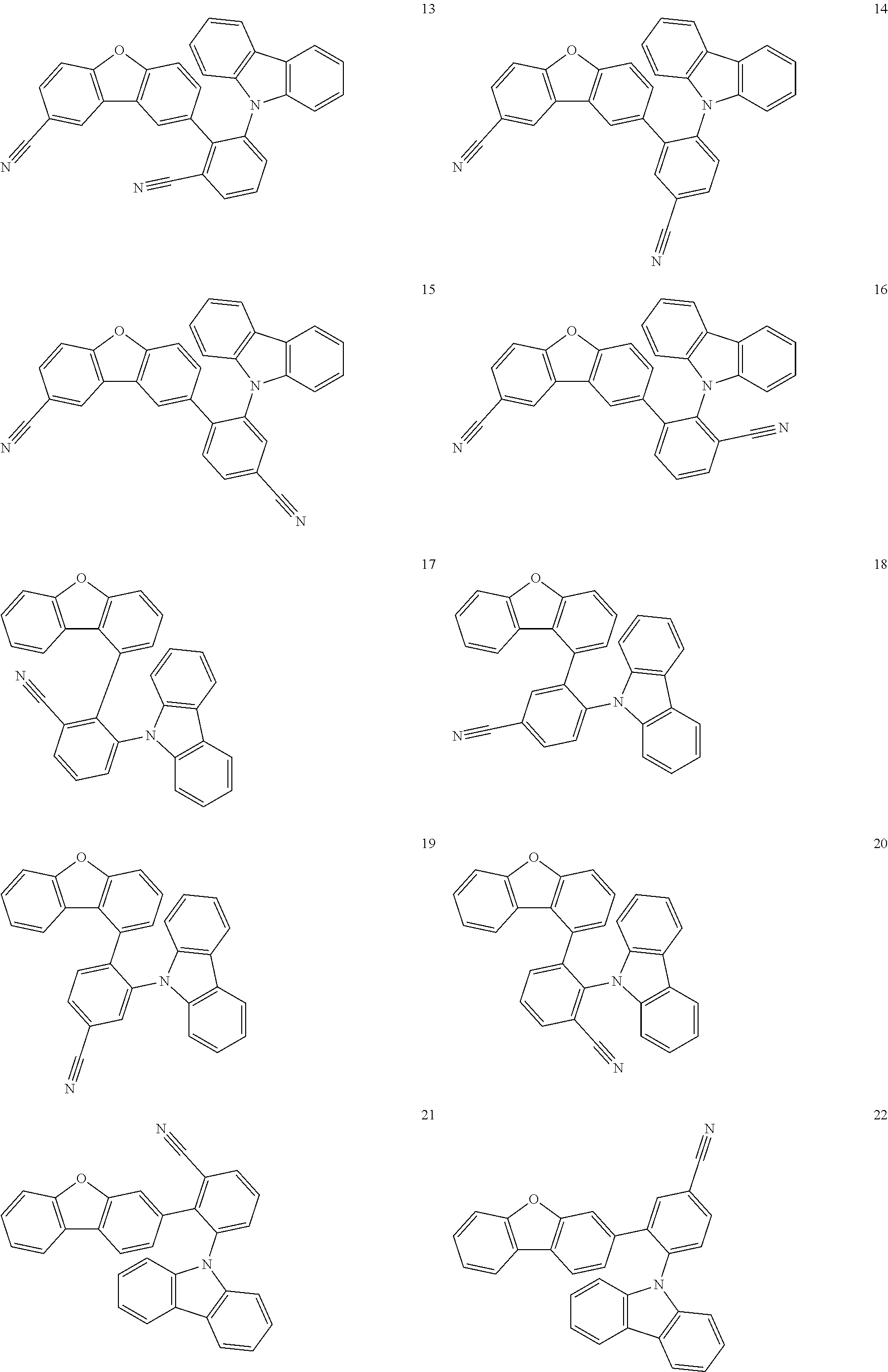

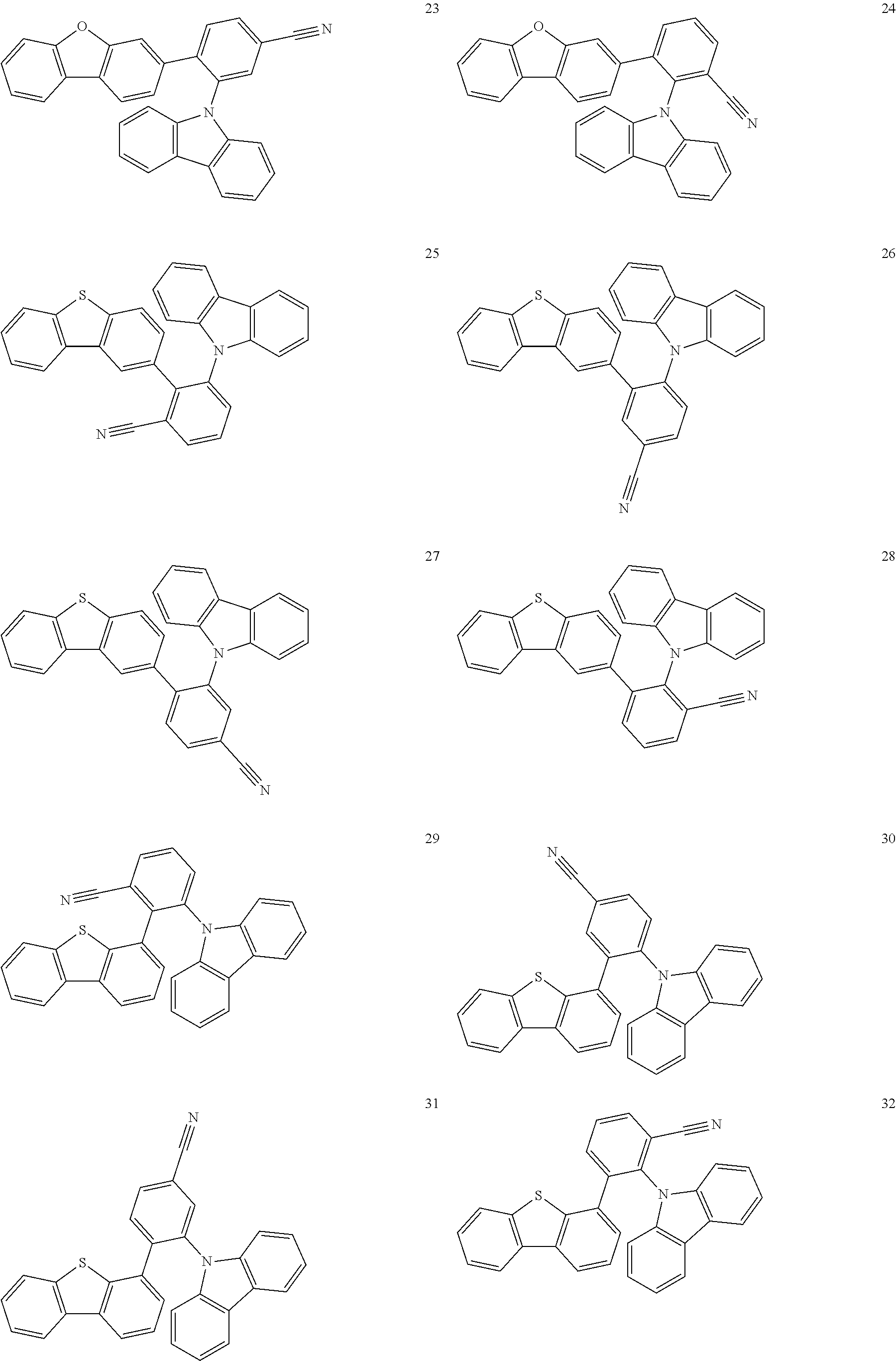

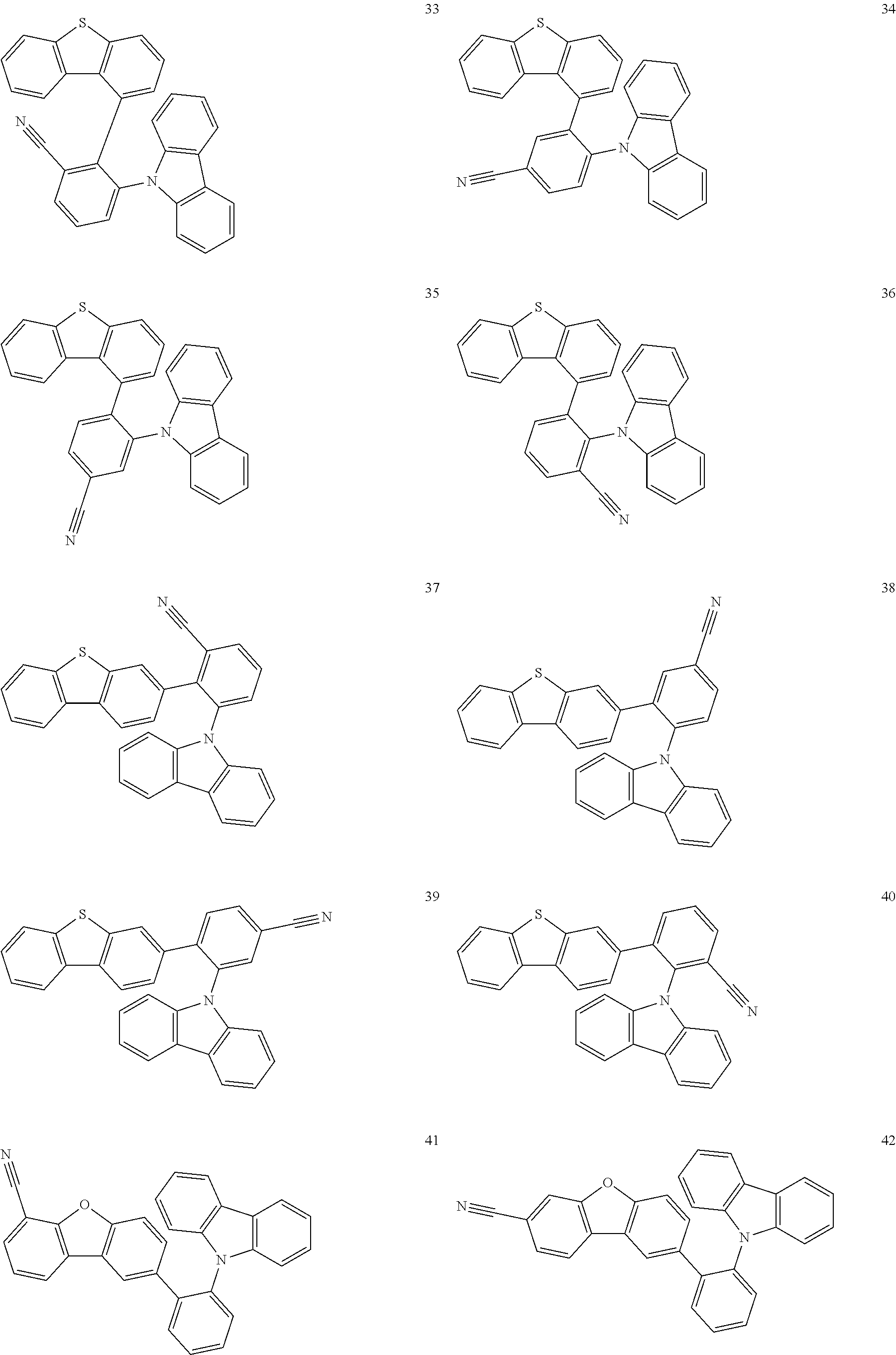

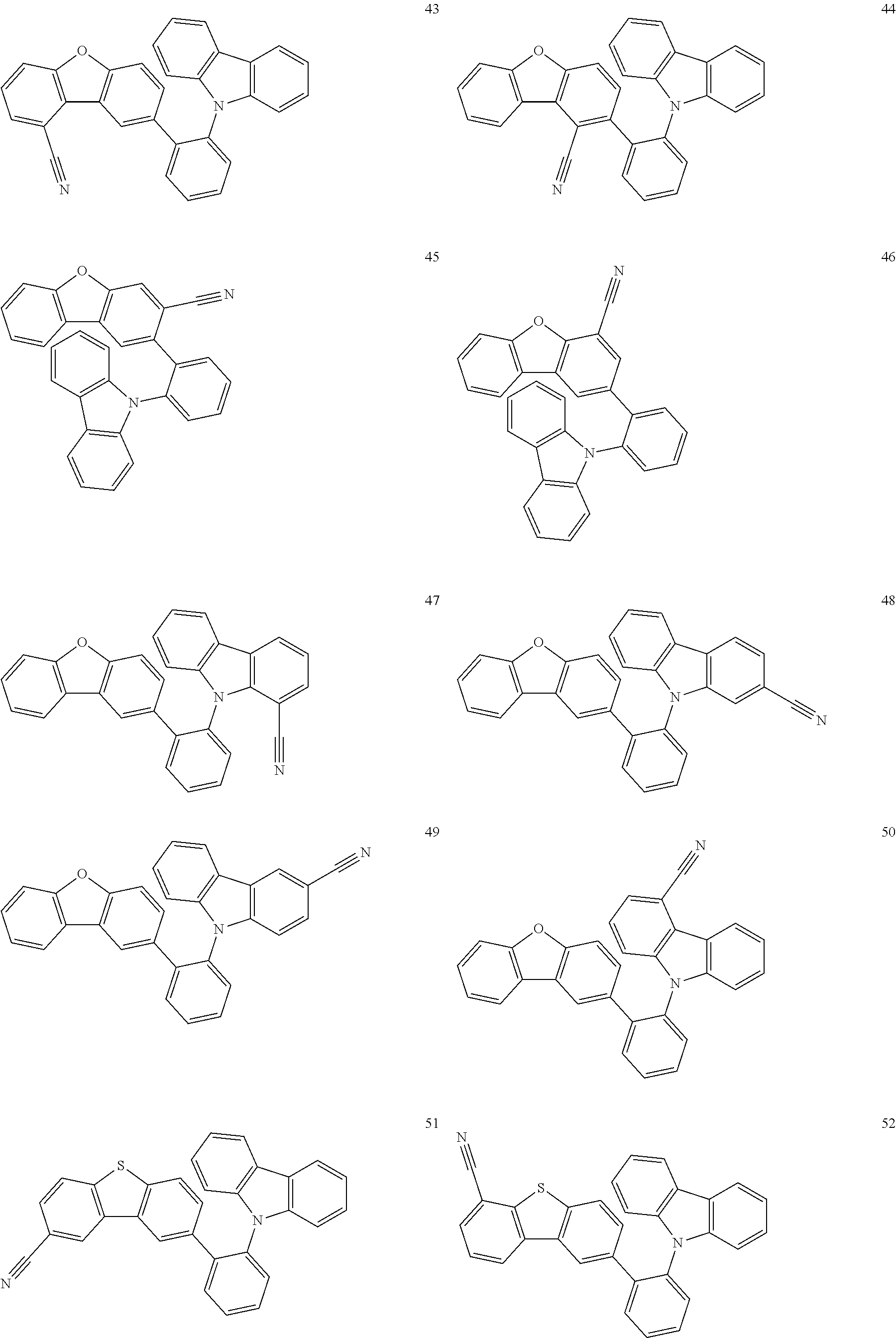

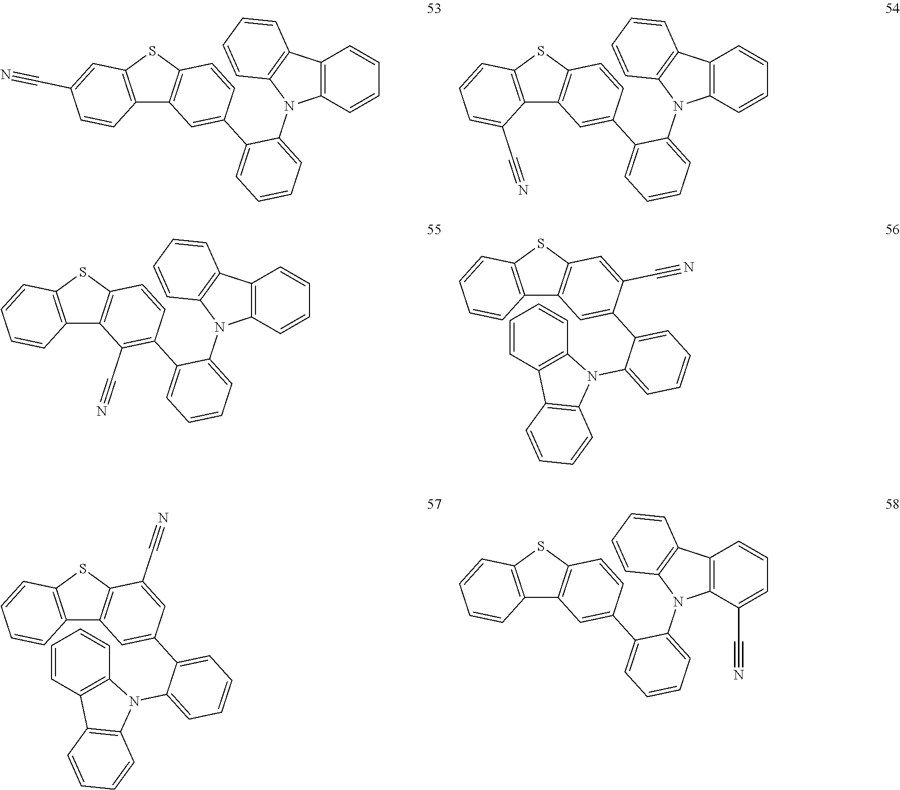 [SMILES: N#Cc1ccc2c(c1)sc1ccc(-c3ccccc3-n3c4ccccc4c4ccccc43)cc12 ; N#Cc1cccc2sc3ccc(-c4ccccc4-n4c5ccccc5c5ccccc54)cc3c12 ; N#Cc1c(-c2ccccc2-n2c3ccccc3c3ccccc32)ccc2sc3ccccc3c12 ; N#Cc1cc2sc3ccccc3c2cc1-c1ccccc1-n1c2ccccc2c2ccccc21 ; N#Cc1cc(-c2ccccc2-n2c3ccccc3c3ccccc32)cc2c1sc1ccccc12 ; N#Cc1cccc2c3ccccc3n(-c3ccccc3-c3ccc4sc5ccccc5c4c3)c12]

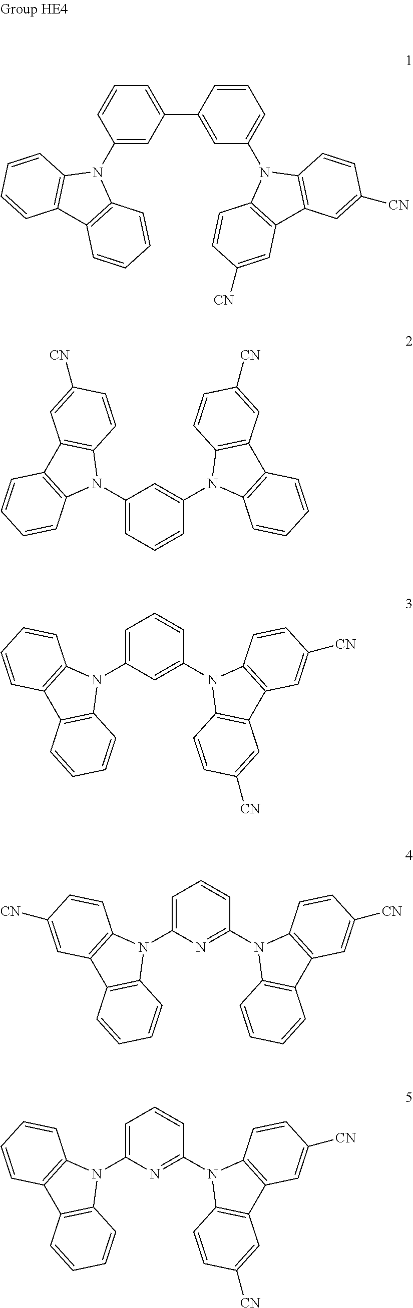

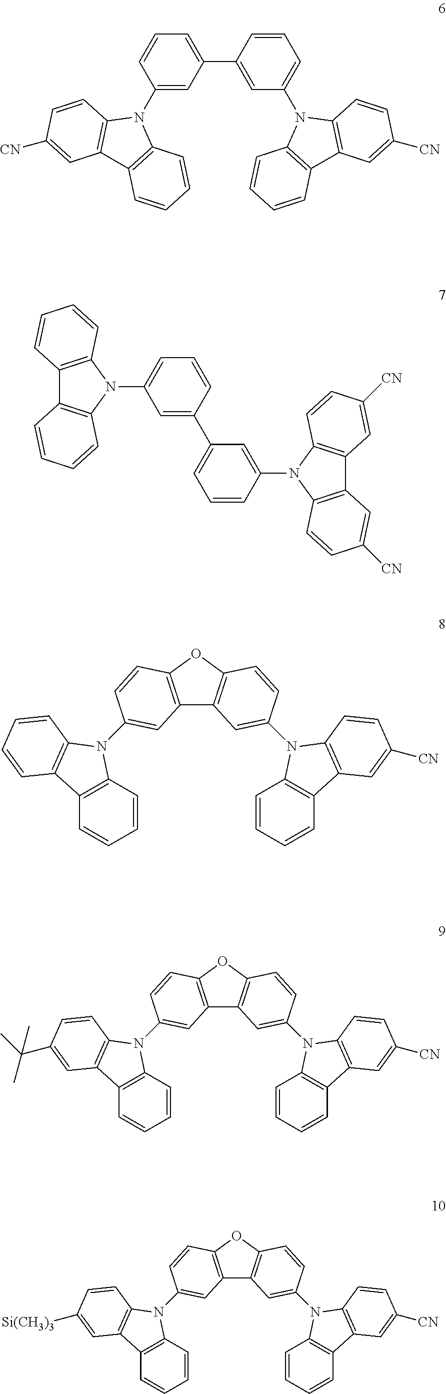

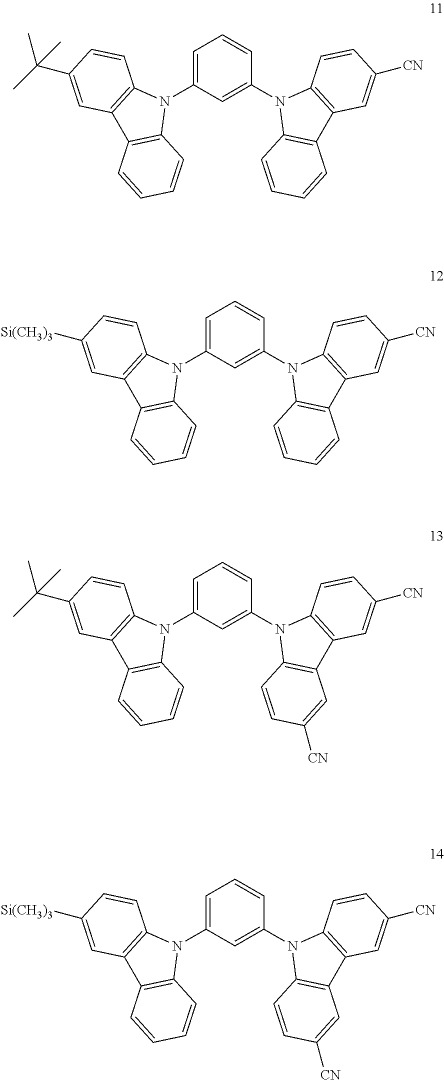

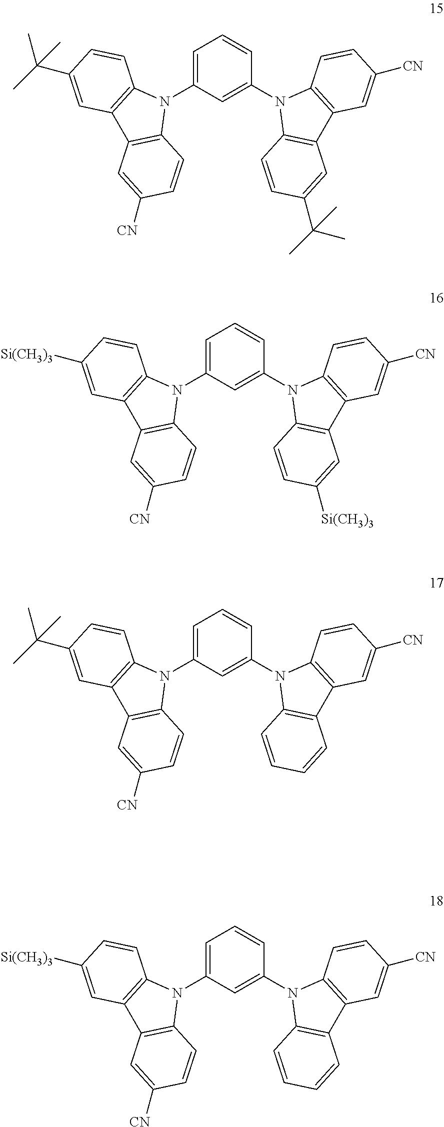

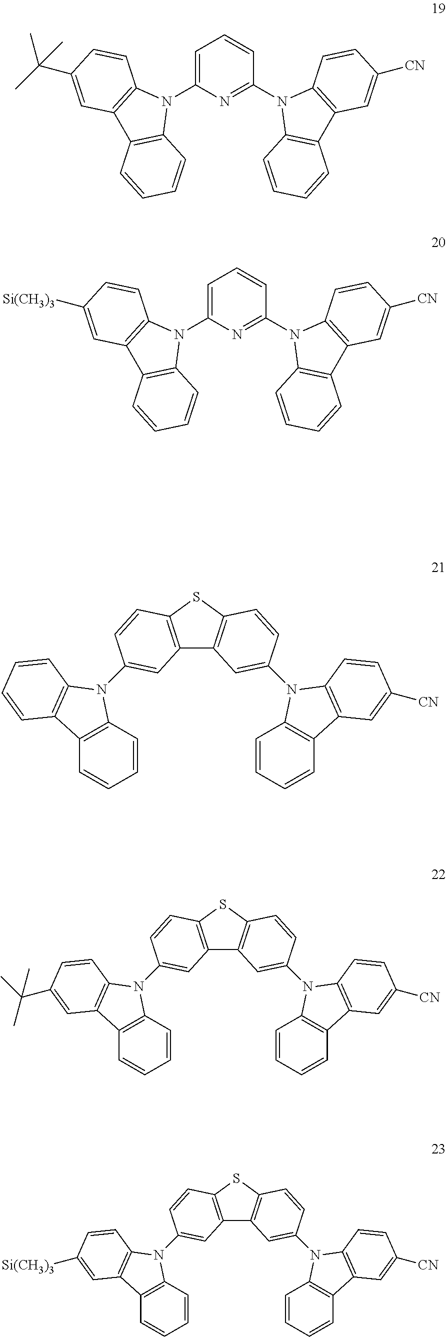

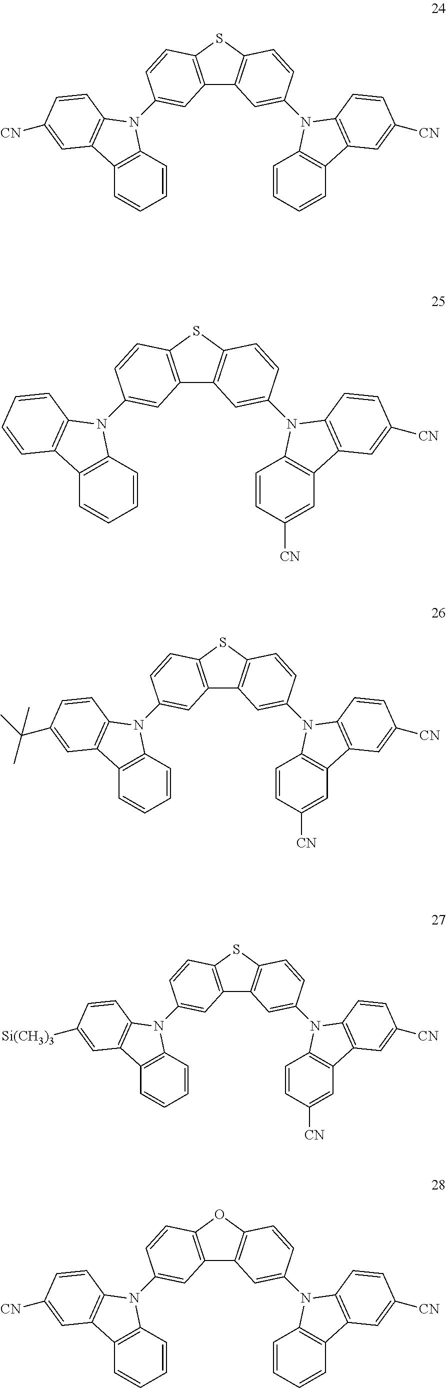

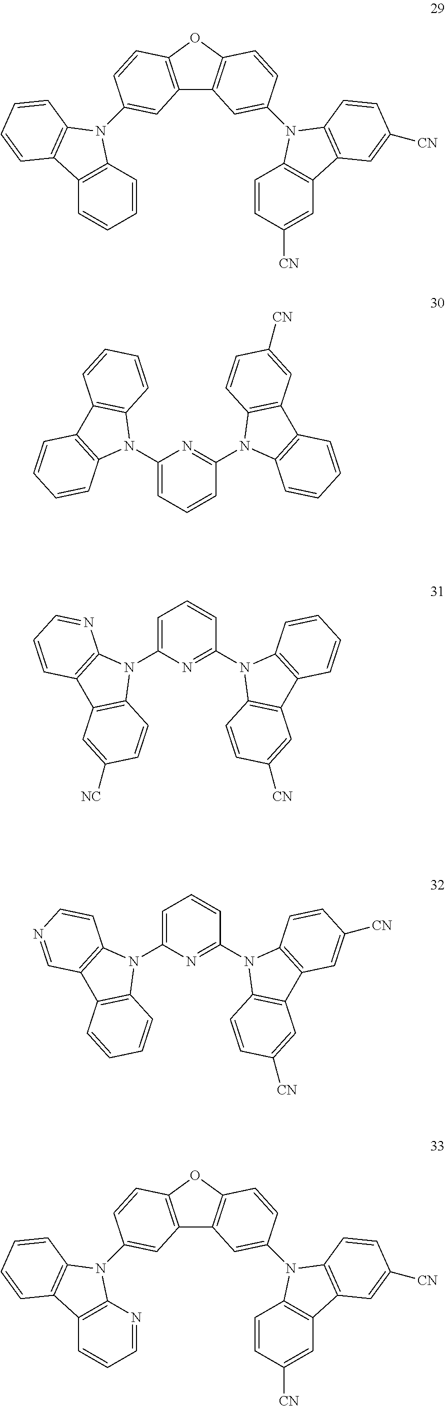 [SMILES: N#Cc1ccc2c(c1)c1cc(C#N)ccc1n2-c1ccc2oc3ccc(-n4c5ccccc5c5ccccc54)cc3c2c1 ; N#Cc1ccc2c(c1)c1ccccc1n2-c1cccc(-n2c3ccccc3c3ccccc32)n1 ; N#Cc1ccc2c(c1)c1ccccc1n2-c1cccc(-n2c3ccc(C#N)cc3c3cccnc32)n1 ; N#Cc1ccc2c(c1)c1cc(C#N)ccc1n2-c1cccc(-n2c3ccccc3c3cnccc32)n1 ; N#Cc1ccc2c(c1)c1cc(C#N)ccc1n2-c1ccc2oc3ccc(-n4c5ccccc5c5cccnc54)cc3c2c1]

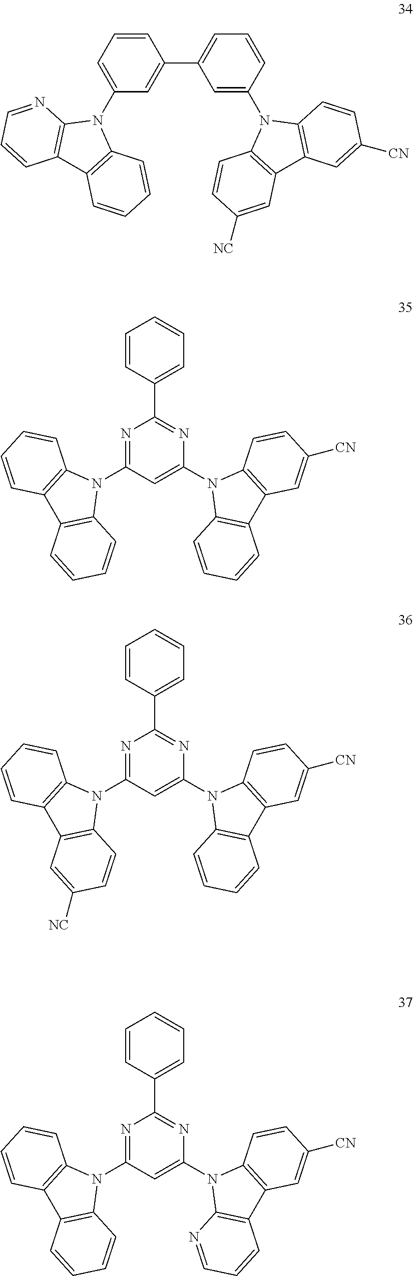 [SMILES: N#Cc1ccc2c(c1)c1cc(C#N)ccc1n2-c1cccc(-c2cccc(-n3c4ccccc4c4cccnc43)c2)c1 ; N#Cc1ccc2c(c1)c1ccccc1n2-c1cc(-n2c3ccccc3c3ccccc32)nc(-c2ccccc2)n1 ; N#Cc1ccc2c(c1)c1ccccc1n2-c1cc(-n2c3ccccc3c3cc(C#N)ccc32)nc(-c2ccccc2)n1 ; N#Cc1ccc2c(c1)c1cccnc1n2-c1cc(-n2c3ccccc3c3ccccc32)nc(-c2ccccc2)n1]

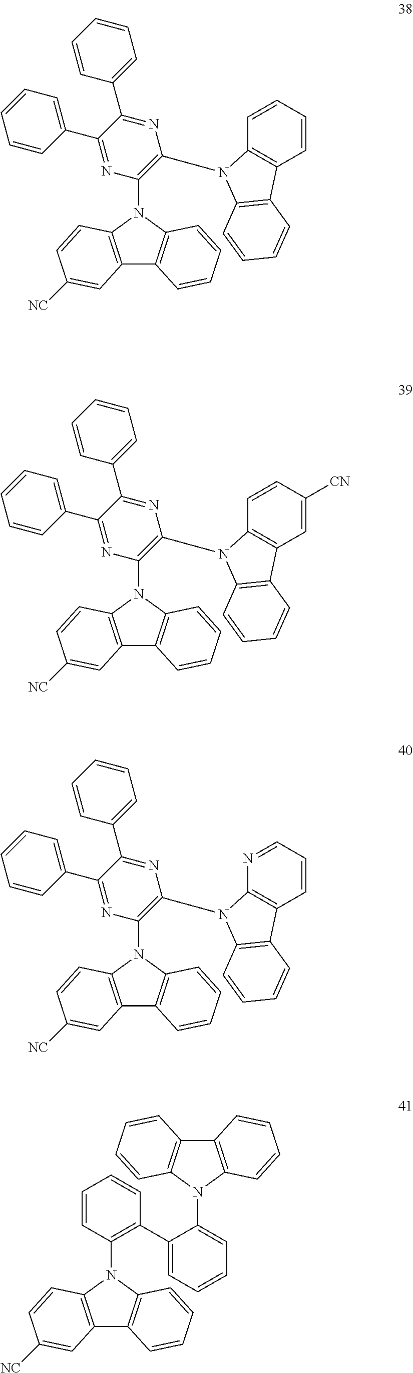 [SMILES: N#Cc1ccc2c(c1)c1ccccc1n2-c1nc(-c2ccccc2)c(-c2ccccc2)nc1-n1c2ccccc2c2ccccc21 ; N#Cc1ccc2c(c1)c1ccccc1n2-c1nc(-c2ccccc2)c(-c2ccccc2)nc1-n1c2ccccc2c2cc(C#N)ccc21 ; N#Cc1ccc2c(c1)c1ccccc1n2-c1nc(-c2ccccc2)c(-c2ccccc2)nc1-n1c2ccccc2c2cccnc21 ; N#Cc1ccc2c(c1)c1ccccc1n2-c1ccccc1-c1ccccc1-n1c2ccccc2c2ccccc21]

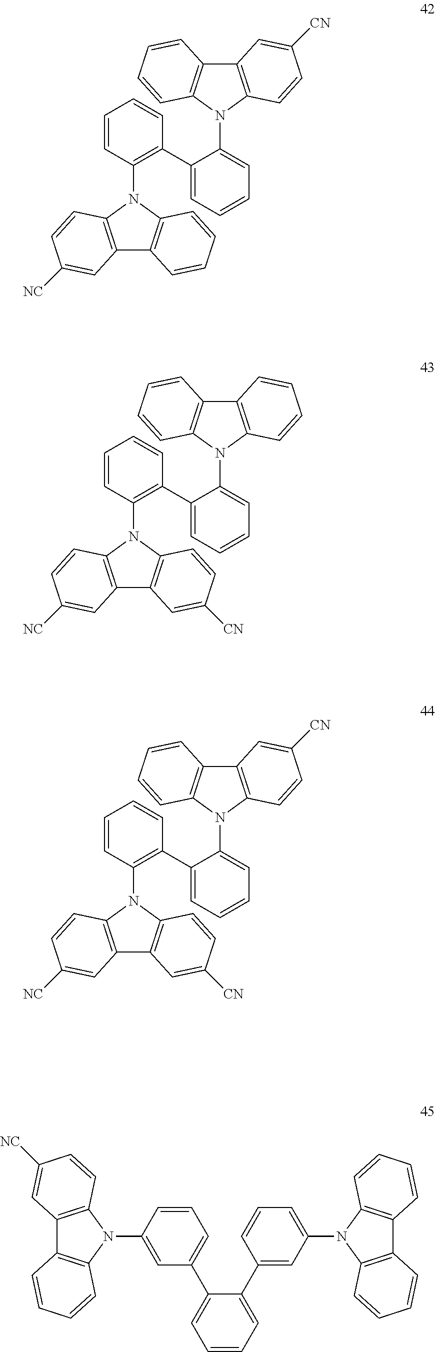 [SMILES: N#Cc1ccc2c(c1)c1ccccc1n2-c1ccccc1-c1ccccc1-n1c2ccccc2c2cc(C#N)ccc21 ; N#Cc1ccc2c(c1)c1cc(C#N)ccc1n2-c1ccccc1-c1ccccc1-n1c2ccccc2c2ccccc21 ; N#Cc1ccc2c(c1)c1ccccc1n2-c1ccccc1-c1ccccc1-n1c2ccc(C#N)cc2c2cc(C#N)ccc21 ; N#Cc1ccc2c(c1)c1ccccc1n2-c1cccc(-c2ccccc2-c2cccc(-n3c4ccccc4c4ccccc43)c2)c1]

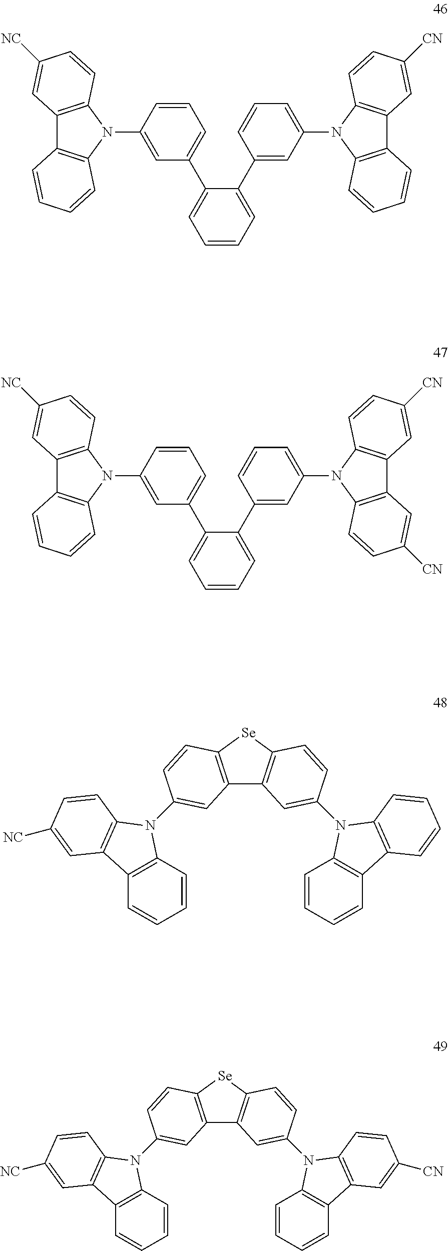 [SMILES: N#Cc1ccc2c(c1)c1ccccc1n2-c1cccc(-c2ccccc2-c2cccc(-n3c4ccccc4c4cc(C#N)ccc43)c2)c1 ; N#Cc1ccc2c(c1)c1ccccc1n2-c1cccc(-c2ccccc2-c2cccc(-n3c4ccc(C#N)cc4c4cc(C#N)ccc43)c2)c1 ; N#Cc1ccc2c(c1)c1ccccc1n2-c1ccc2[se]c3ccc(-n4c5ccccc5c5ccccc54)cc3c2c1 ; N#Cc1ccc2c(c1)c1ccccc1n2-c1ccc2[se]c3ccc(-n4c5ccccc5c5cc(C#N)ccc54)cc3c2c1]

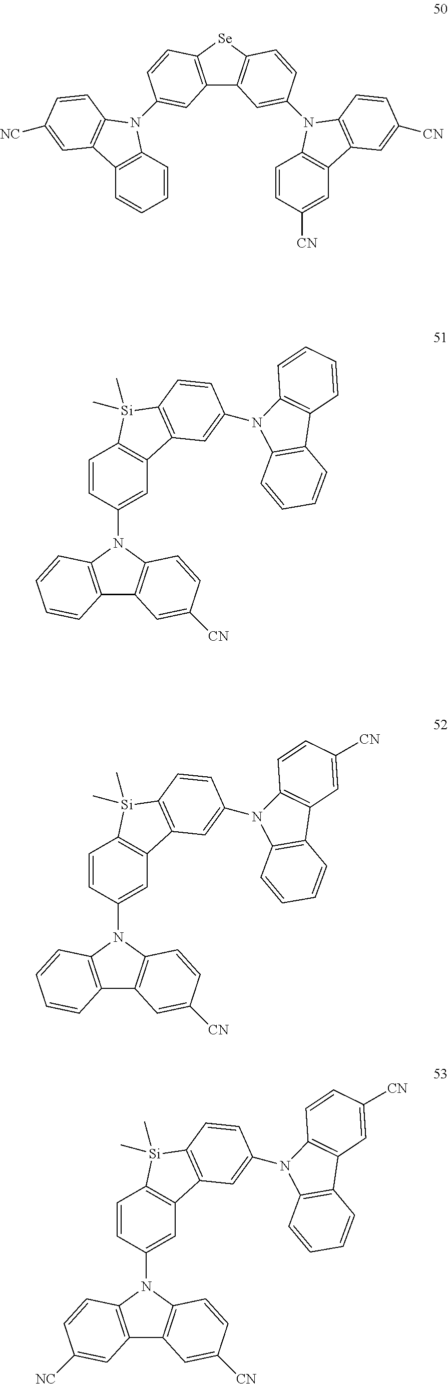 [SMILES: N#Cc1ccc2c(c1)c1ccccc1n2-c1ccc2[se]c3ccc(-n4c5ccc(C#N)cc5c5cc(C#N)ccc54)cc3c2c1 ; C[Si]1(C)c2ccc(-n3c4ccccc4c4ccccc43)cc2-c2cc(-n3c4ccccc4c4cc(C#N)ccc43)ccc21 ; C[Si]1(C)c2ccc(-n3c4ccccc4c4cc(C#N)ccc43)cc2-c2cc(-n3c4ccccc4c4cc(C#N)ccc43)ccc21 ; C[Si]1(C)c2ccc(-n3c4ccccc4c4cc(C#N)ccc43)cc2-c2cc(-n3c4ccc(C#N)cc4c4cc(C#N)ccc43)ccc21]

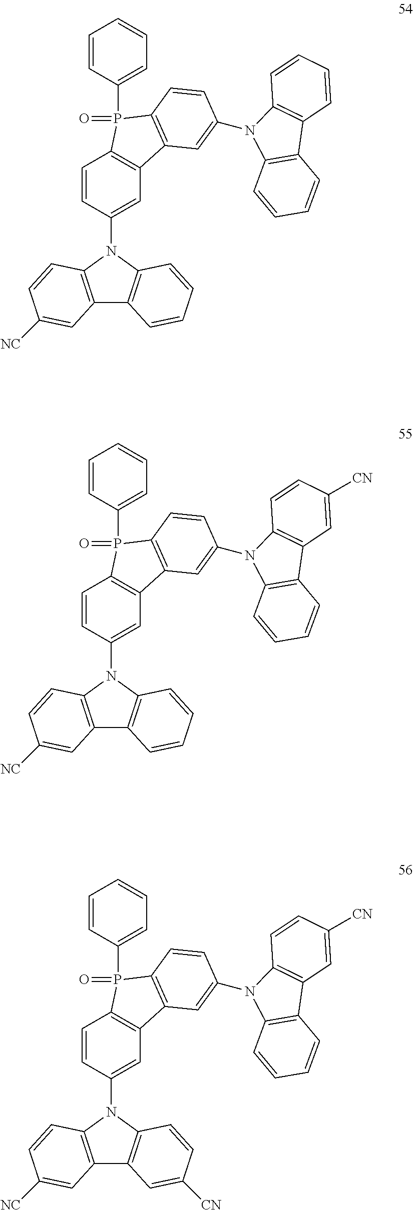 [SMILES: N#Cc1ccc2c(c1)c1ccccc1n2-c1ccc2c(c1)-c1cc(-n3c4ccccc4c4ccccc43)ccc1P2(=O)c1ccccc1 ; N#Cc1ccc2c(c1)c1ccccc1n2-c1ccc2c(c1)-c1cc(-n3c4ccccc4c4cc(C#N)ccc43)ccc1P2(=O)c1ccccc1 ; N#Cc1ccc2c(c1)c1ccccc1n2-c1ccc2c(c1)-c1cc(-n3c4ccc(C#N)cc4c4cc(C#N)ccc43)ccc1P2(=O)c1ccccc1]

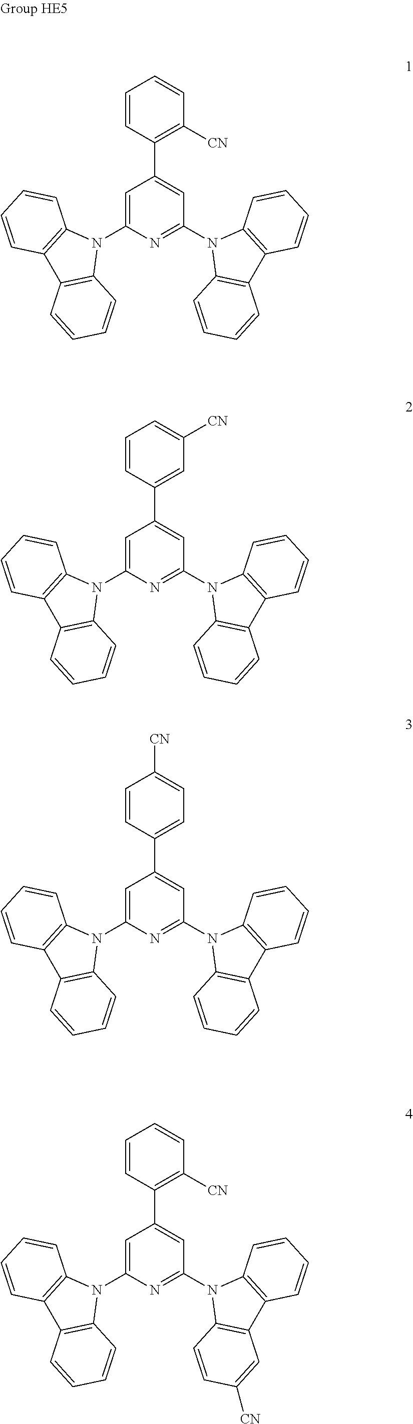 [SMILES: N#Cc1ccccc1-c1cc(-n2c3ccccc3c3ccccc32)nc(-n2c3ccccc3c3ccccc32)c1 ; N#Cc1cccc(-c2cc(-n3c4ccccc4c4ccccc43)nc(-n3c4ccccc4c4ccccc43)c2)c1 ; N#Cc1ccc(-c2cc(-n3c4ccccc4c4ccccc43)nc(-n3c4ccccc4c4ccccc43)c2)cc1 ; N#Cc1ccc2c(c1)c1ccccc1n2-c1cc(-c2ccccc2C#N)cc(-n2c3ccccc3c3ccccc32)n1]

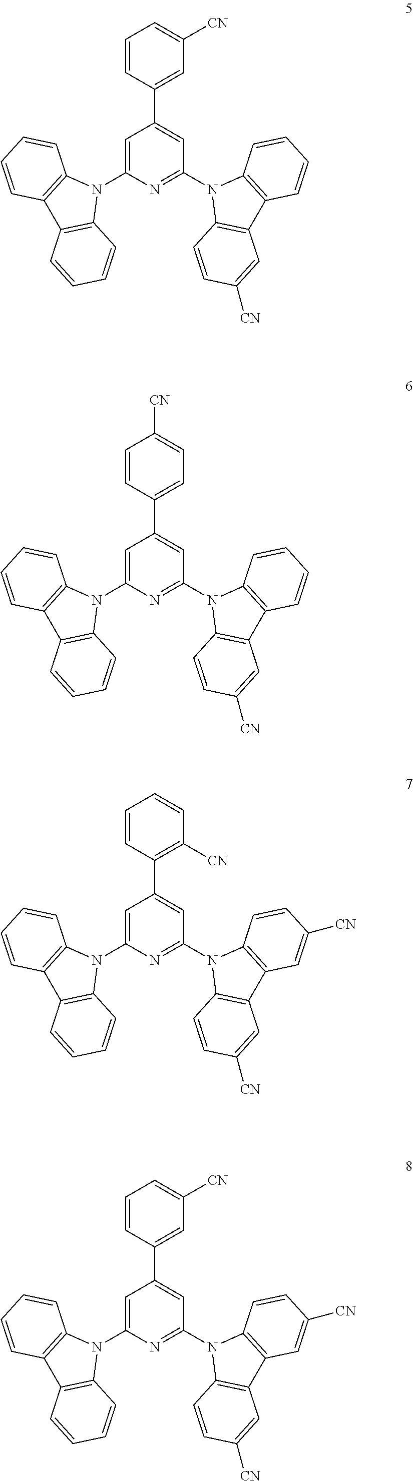 [SMILES: N#Cc1cccc(-c2cc(-n3c4ccccc4c4ccccc43)nc(-n3c4ccccc4c4cc(C#N)ccc43)c2)c1 ; N#Cc1ccc(-c2cc(-n3c4ccccc4c4ccccc43)nc(-n3c4ccccc4c4cc(C#N)ccc43)c2)cc1 ; N#Cc1ccc2c(c1)c1cc(C#N)ccc1n2-c1cc(-c2ccccc2C#N)cc(-n2c3ccccc3c3ccccc32)n1 ; N#Cc1cccc(-c2cc(-n3c4ccccc4c4ccccc43)nc(-n3c4ccc(C#N)cc4c4cc(C#N)ccc43)c2)c1]

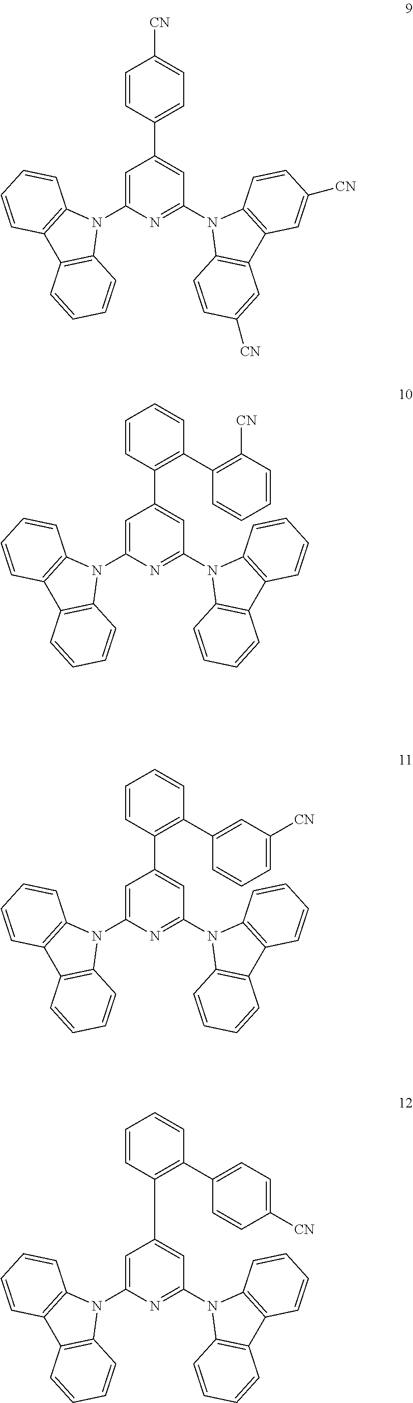 [SMILES: N#Cc1ccc(-c2cc(-n3c4ccccc4c4ccccc43)nc(-n3c4ccc(C#N)cc4c4cc(C#N)ccc43)c2)cc1 ; N#Cc1ccccc1-c1ccccc1-c1cc(-n2c3ccccc3c3ccccc32)nc(-n2c3ccccc3c3ccccc32)c1 ; N#Cc1cccc(-c2ccccc2-c2cc(-n3c4ccccc4c4ccccc43)nc(-n3c4ccccc4c4ccccc43)c2)c1 ; N#Cc1ccc(-c2ccccc2-c2cc(-n3c4ccccc4c4ccccc43)nc(-n3c4ccccc4c4ccccc43)c2)cc1]

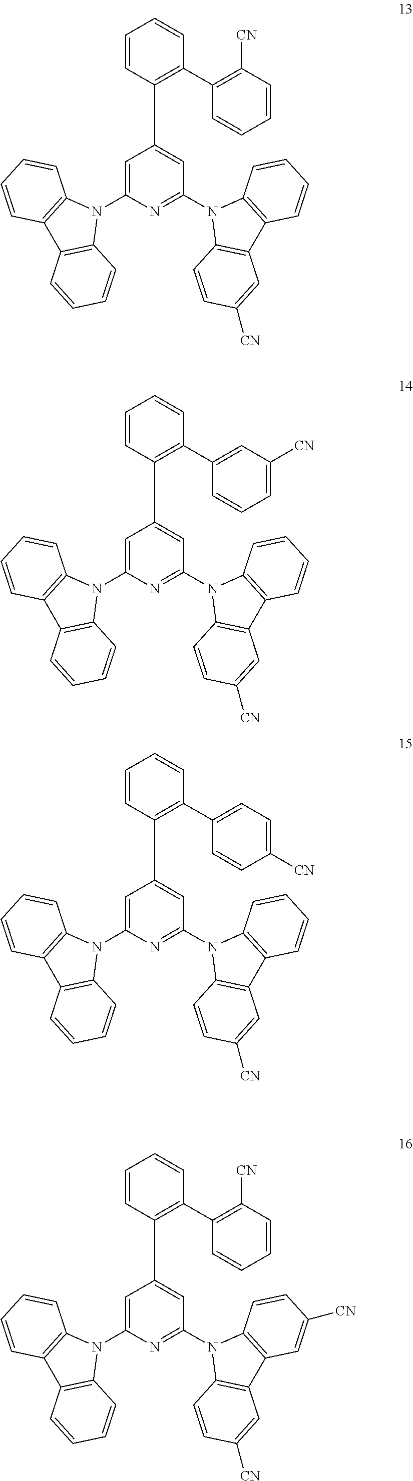 [SMILES: N#Cc1ccc2c(c1)c1ccccc1n2-c1cc(-c2ccccc2-c2ccccc2C#N)cc(-n2c3ccccc3c3ccccc32)n1 ; N#Cc1cccc(-c2ccccc2-c2cc(-n3c4ccccc4c4ccccc43)nc(-n3c4ccccc4c4cc(C#N)ccc43)c2)c1 ; N#Cc1ccc(-c2ccccc2-c2cc(-n3c4ccccc4c4ccccc43)nc(-n3c4ccccc4c4cc(C#N)ccc43)c2)cc1 ; N#Cc1ccc2c(c1)c1cc(C#N)ccc1n2-c1cc(-c2ccccc2-c2ccccc2C#N)cc(-n2c3ccccc3c3ccccc32)n1]

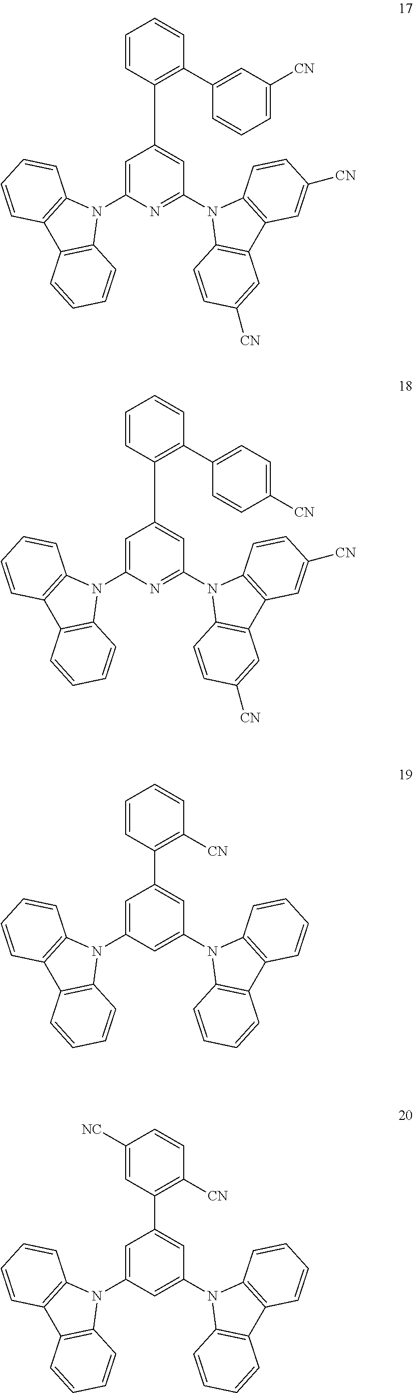 [SMILES: N#Cc1cccc(-c2ccccc2-c2cc(-n3c4ccccc4c4ccccc43)nc(-n3c4ccc(C#N)cc4c4cc(C#N)ccc43)c2)c1 ; N#Cc1ccc(-c2ccccc2-c2cc(-n3c4ccccc4c4ccccc43)nc(-n3c4ccc(C#N)cc4c4cc(C#N)ccc43)c2)cc1 ; N#Cc1ccccc1-c1cc(-n2c3ccccc3c3ccccc32)cc(-n2c3ccccc3c3ccccc32)c1 ; N#Cc1ccc(C#N)c(-c2cc(-n3c4ccccc4c4ccccc43)cc(-n3c4ccccc4c4ccccc43)c2)c1]

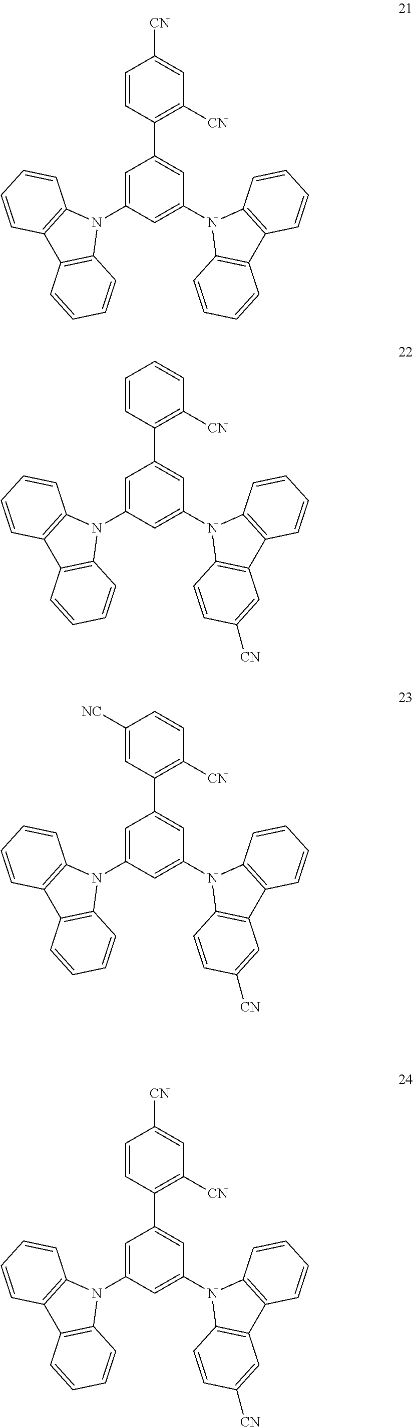 [SMILES: N#Cc1ccc(-c2cc(-n3c4ccccc4c4ccccc43)cc(-n3c4ccccc4c4ccccc43)c2)c(C#N)c1 ; N#Cc1ccc2c(c1)c1ccccc1n2-c1cc(-c2ccccc2C#N)cc(-n2c3ccccc3c3ccccc32)c1 ; N#Cc1ccc(C#N)c(-c2cc(-n3c4ccccc4c4ccccc43)cc(-n3c4ccccc4c4cc(C#N)ccc43)c2)c1 ; N#Cc1ccc(-c2cc(-n3c4ccccc4c4ccccc43)cc(-n3c4ccccc4c4cc(C#N)ccc43)c2)c(C#N)c1]

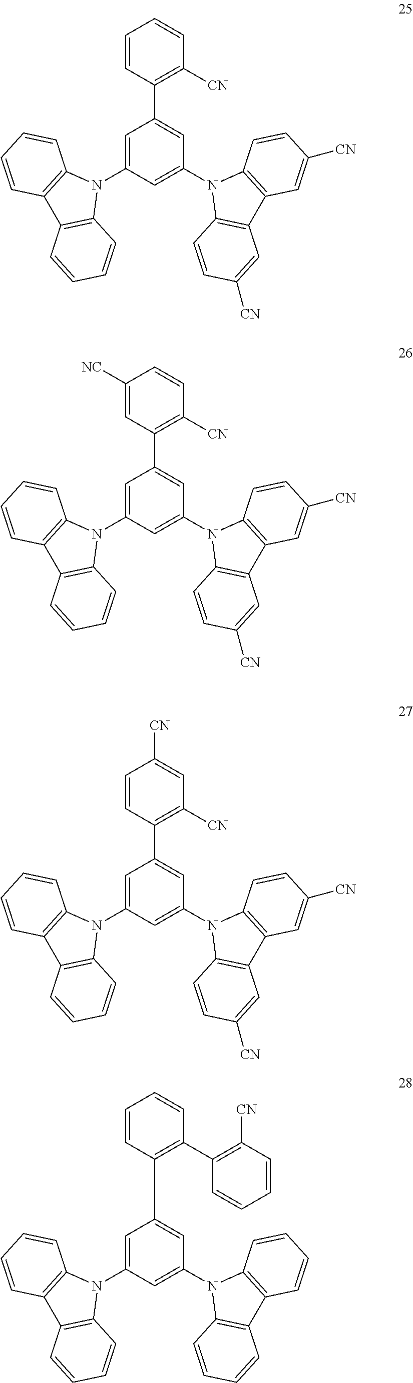 [SMILES: N#Cc1ccc2c(c1)c1cc(C#N)ccc1n2-c1cc(-c2ccccc2C#N)cc(-n2c3ccccc3c3ccccc32)c1 ; N#Cc1ccc(C#N)c(-c2cc(-n3c4ccccc4c4ccccc43)cc(-n3c4ccc(C#N)cc4c4cc(C#N)ccc43)c2)c1 ; N#Cc1ccc(-c2cc(-n3c4ccccc4c4ccccc43)cc(-n3c4ccc(C#N)cc4c4cc(C#N)ccc43)c2)c(C#N)c1 ; N#Cc1ccccc1-c1ccccc1-c1cc(-n2c3ccccc3c3ccccc32)cc(-n2c3ccccc3c3ccccc32)c1]

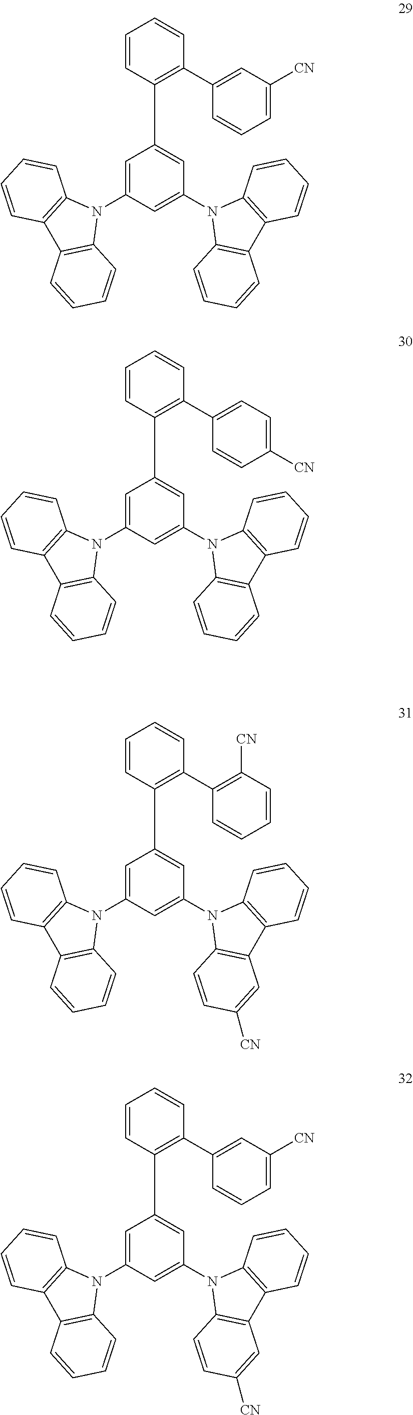 [SMILES: N#Cc1cccc(-c2ccccc2-c2cc(-n3c4ccccc4c4ccccc43)cc(-n3c4ccccc4c4ccccc43)c2)c1 ; N#Cc1ccc(-c2ccccc2-c2cc(-n3c4ccccc4c4ccccc43)cc(-n3c4ccccc4c4ccccc43)c2)cc1 ; N#Cc1ccc2c(c1)c1ccccc1n2-c1cc(-c2ccccc2-c2ccccc2C#N)cc(-n2c3ccccc3c3ccccc32)c1 ; N#Cc1cccc(-c2ccccc2-c2cc(-n3c4ccccc4c4ccccc43)cc(-n3c4ccccc4c4cc(C#N)ccc43)c2)c1]

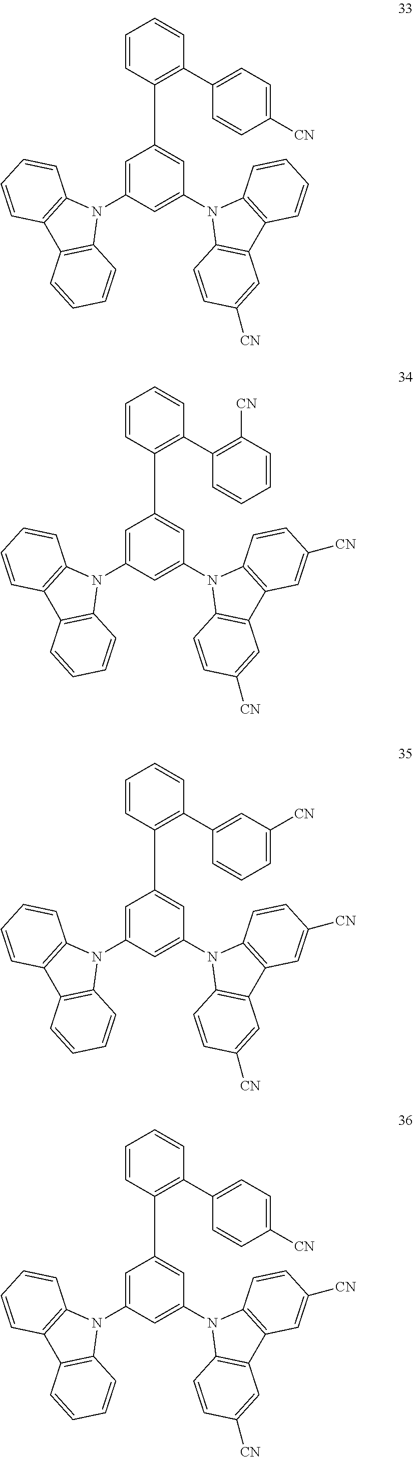 [SMILES: N#Cc1ccc(-c2ccccc2-c2cc(-n3c4ccccc4c4ccccc43)cc(-n3c4ccccc4c4cc(C#N)ccc43)c2)cc1 ; N#Cc1ccc2c(c1)c1cc(C#N)ccc1n2-c1cc(-c2ccccc2-c2ccccc2C#N)cc(-n2c3ccccc3c3ccccc32)c1 ; N#Cc1cccc(-c2ccccc2-c2cc(-n3c4ccccc4c4ccccc43)cc(-n3c4ccc(C#N)cc4c4cc(C#N)ccc43)c2)c1 ; N#Cc1ccc(-c2ccccc2-c2cc(-n3c4ccccc4c4ccccc43)cc(-n3c4ccc(C#N)cc4c4cc(C#N)ccc43)c2)cc1]

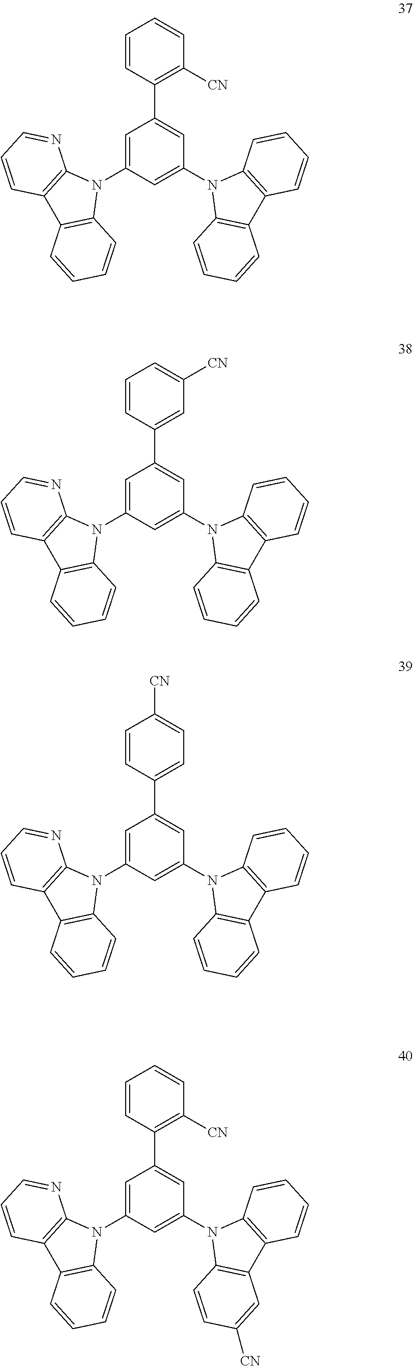 [SMILES: N#Cc1ccccc1-c1cc(-n2c3ccccc3c3ccccc32)cc(-n2c3ccccc3c3cccnc32)c1 ; N#Cc1cccc(-c2cc(-n3c4ccccc4c4ccccc43)cc(-n3c4ccccc4c4cccnc43)c2)c1 ; N#Cc1ccc(-c2cc(-n3c4ccccc4c4ccccc43)cc(-n3c4ccccc4c4cccnc43)c2)cc1 ; N#Cc1ccc2c(c1)c1ccccc1n2-c1cc(-c2ccccc2C#N)cc(-n2c3ccccc3c3cccnc32)c1]

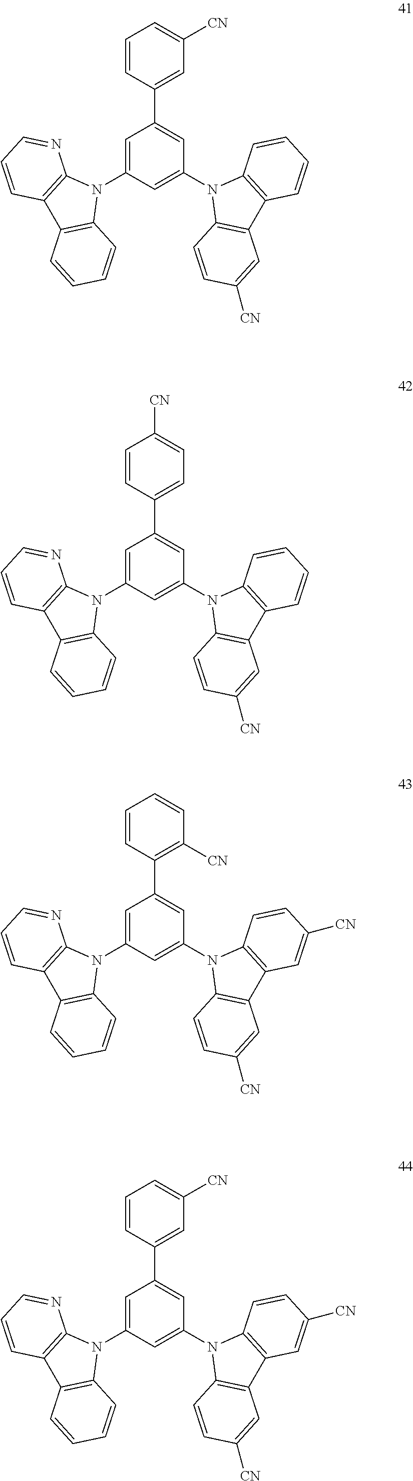 [SMILES: N#Cc1cccc(-c2cc(-n3c4ccccc4c4cc(C#N)ccc43)cc(-n3c4ccccc4c4cccnc43)c2)c1 ; N#Cc1ccc(-c2cc(-n3c4ccccc4c4cc(C#N)ccc43)cc(-n3c4ccccc4c4cccnc43)c2)cc1 ; N#Cc1ccc2c(c1)c1cc(C#N)ccc1n2-c1cc(-c2ccccc2C#N)cc(-n2c3ccccc3c3cccnc32)c1 ; N#Cc1cccc(-c2cc(-n3c4ccc(C#N)cc4c4cc(C#N)ccc43)cc(-n3c4ccccc4c4cccnc43)c2)c1]

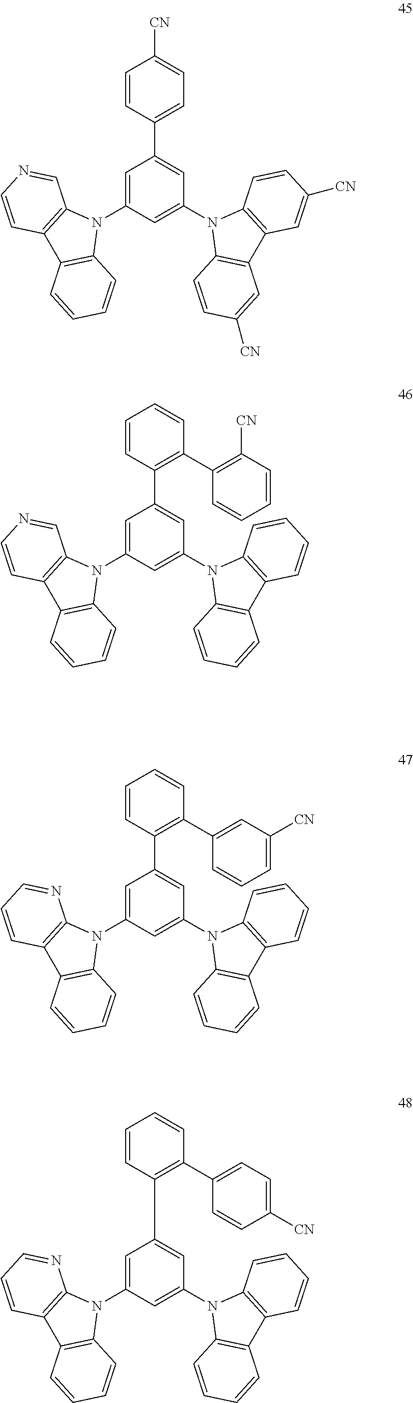 [SMILES: N#Cc1ccc(-c2cc(-n3c4ccc(C#N)cc4c4cc(C#N)ccc43)cc(-n3c4ccccc4c4ccncc43)c2)cc1 ; N#Cc1ccccc1-c1ccccc1-c1cc(-n2c3ccccc3c3ccccc32)cc(-n2c3ccccc3c3ccncc32)c1 ; N#Cc1cccc(-c2ccccc2-c2cc(-n3c4ccccc4c4ccccc43)cc(-n3c4ccccc4c4cccnc43)c2)c1 ; N#Cc1ccc(-c2ccccc2-c2cc(-n3c4ccccc4c4ccccc43)cc(-n3c4ccccc4c4cccnc43)c2)cc1]

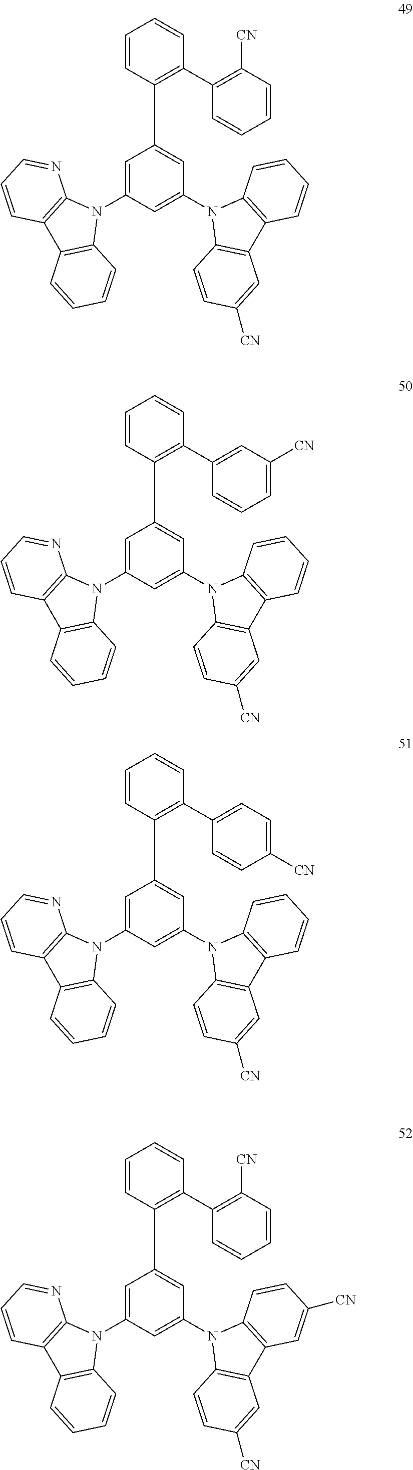 [SMILES: N#Cc1ccc2c(c1)c1ccccc1n2-c1cc(-c2ccccc2-c2ccccc2C#N)cc(-n2c3ccccc3c3cccnc32)c1 ; N#Cc1cccc(-c2ccccc2-c2cc(-n3c4ccccc4c4cc(C#N)ccc43)cc(-n3c4ccccc4c4cccnc43)c2)c1 ; N#Cc1ccc(-c2ccccc2-c2cc(-n3c4ccccc4c4cc(C#N)ccc43)cc(-n3c4ccccc4c4cccnc43)c2)cc1 ; N#Cc1ccc2c(c1)c1cc(C#N)ccc1n2-c1cc(-c2ccccc2-c2ccccc2C#N)cc(-n2c3ccccc3c3cccnc32)c1]

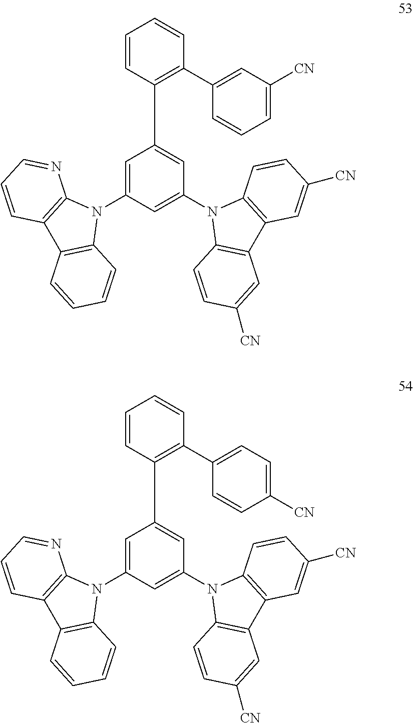 [SMILES: N#Cc1cccc(-c2ccccc2-c2cc(-n3c4ccc(C#N)cc4c4cc(C#N)ccc43)cc(-n3c4ccccc4c4cccnc43)c2)c1 ; N#Cc1ccc(-c2ccccc2-c2cc(-n3c4ccc(C#N)cc4c4cc(C#N)ccc43)cc(-n3c4ccccc4c4cccnc43)c2)cc1]

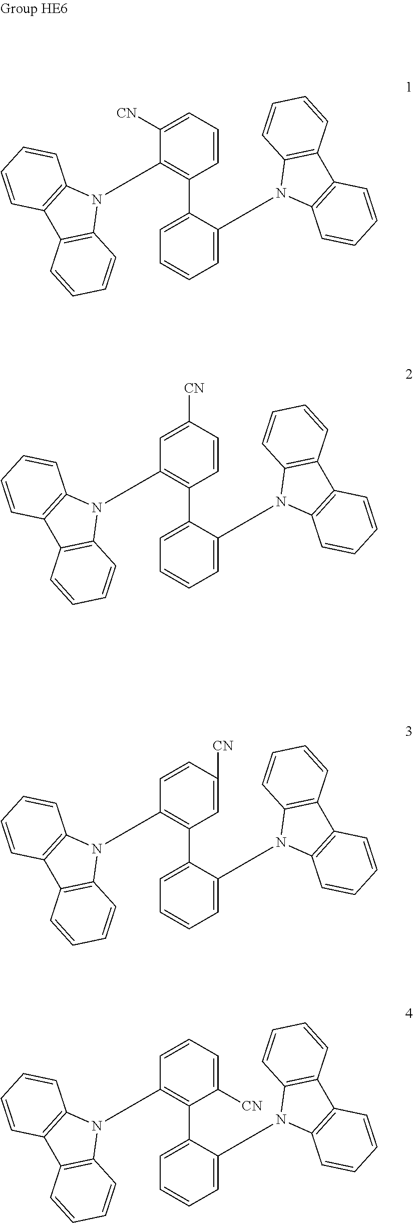

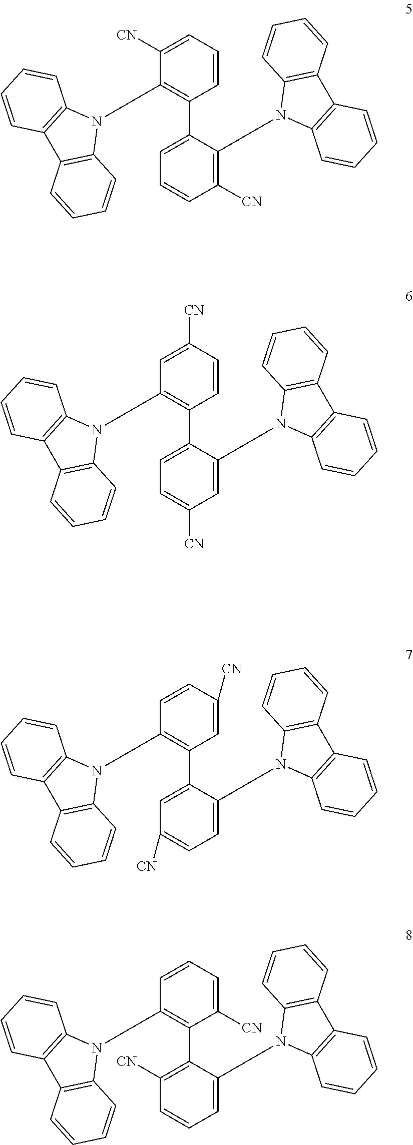

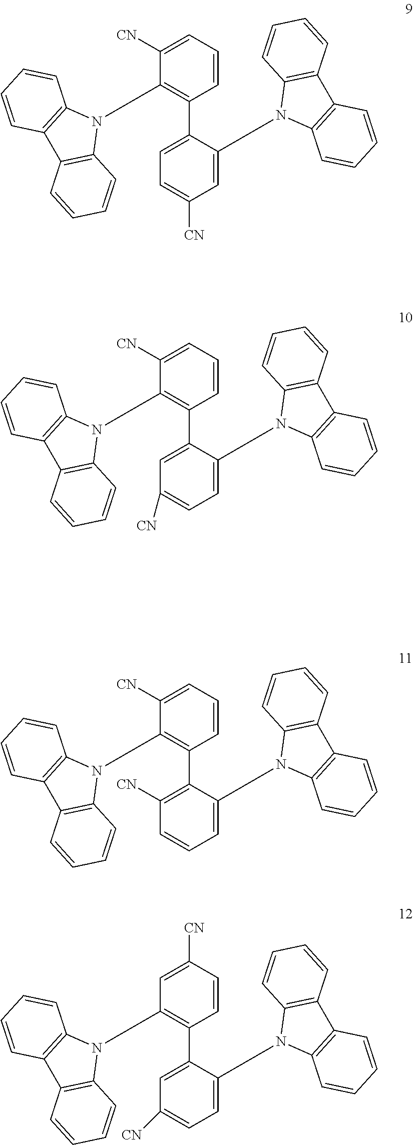

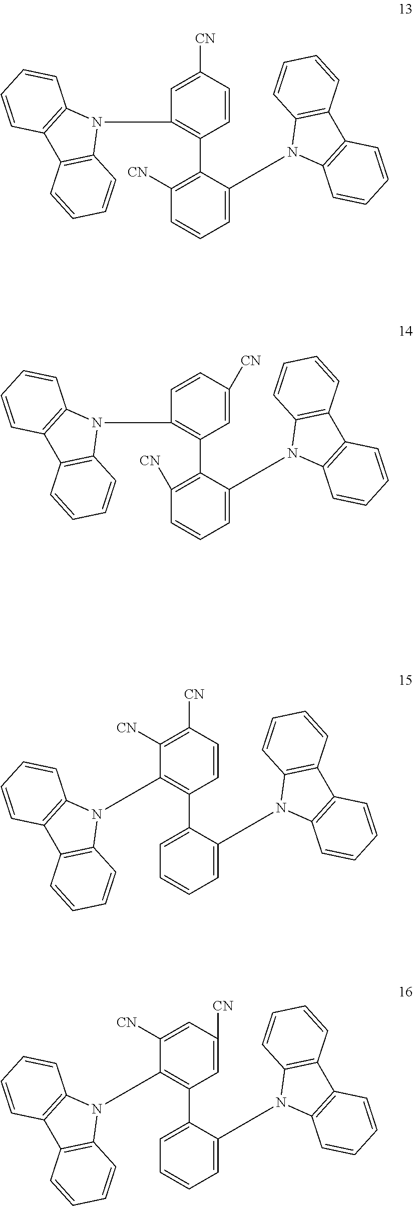

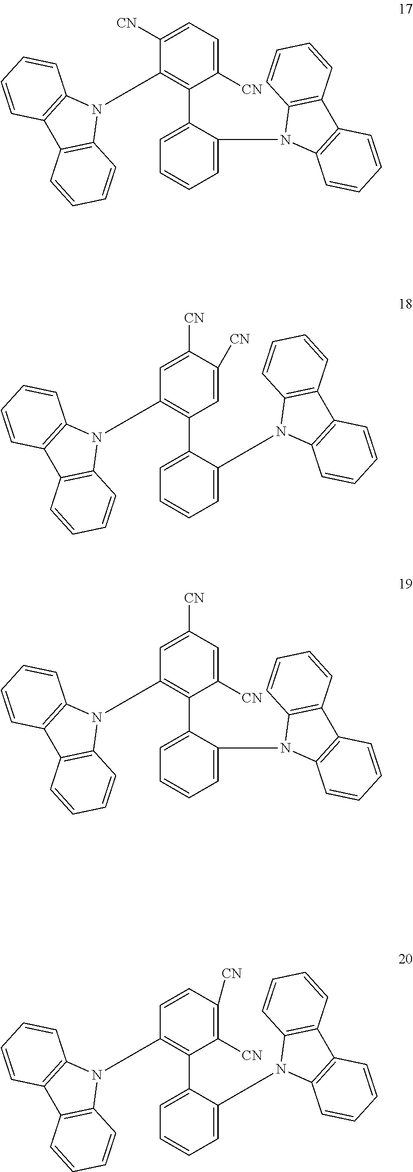

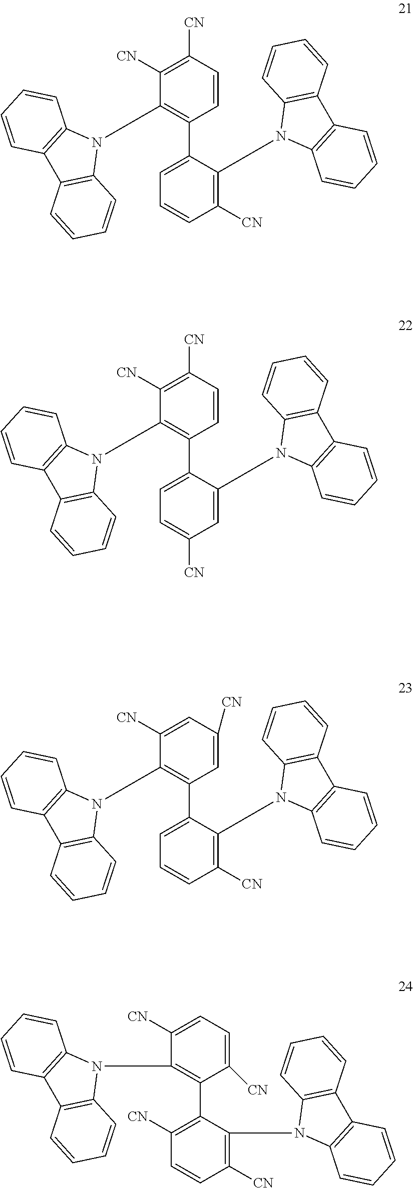

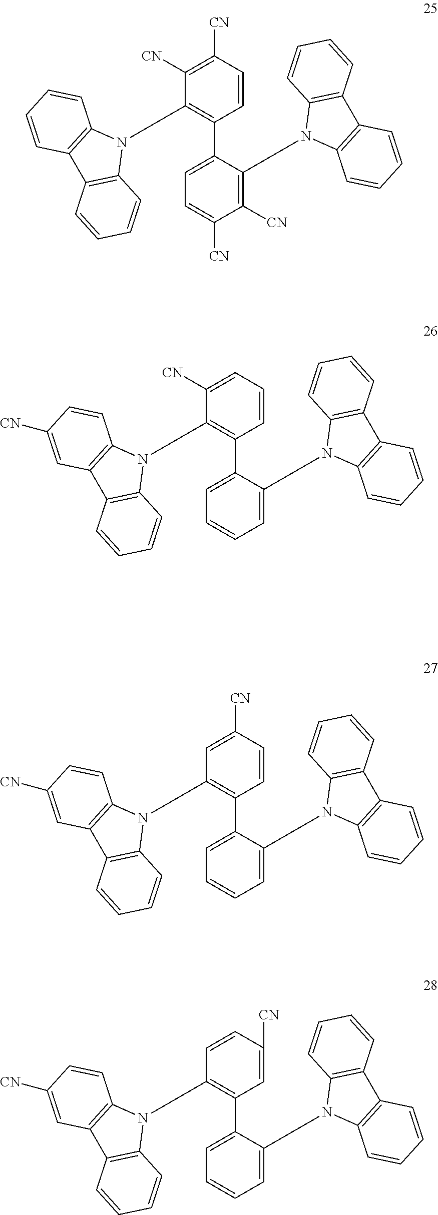

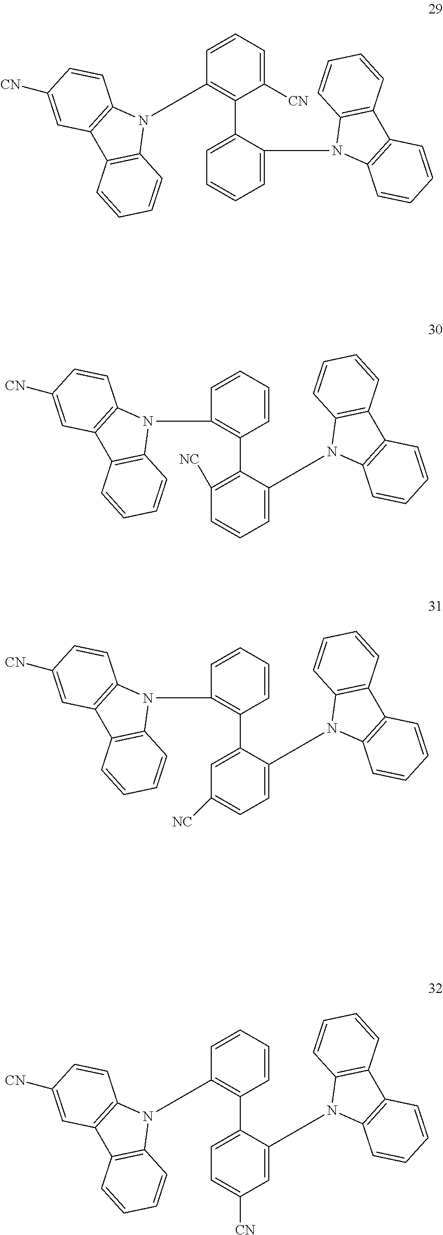

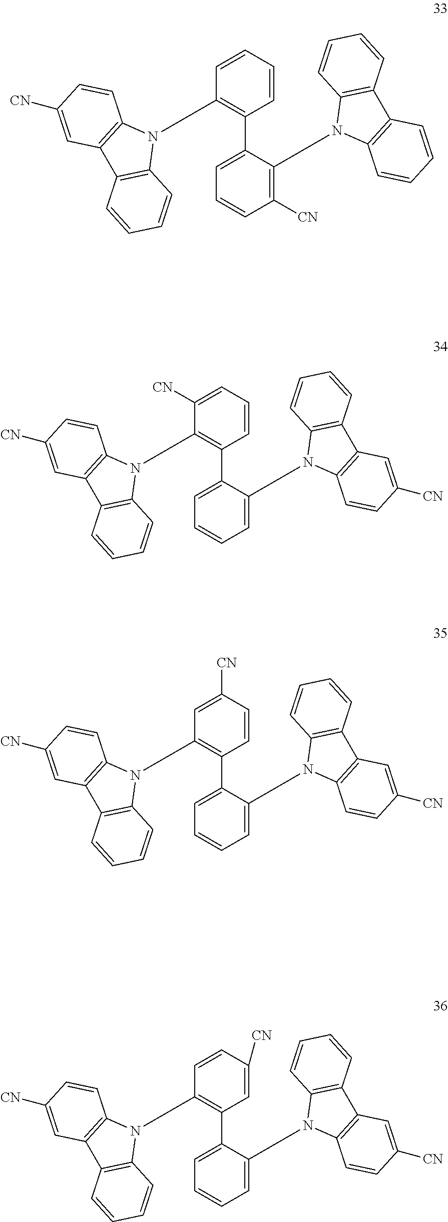

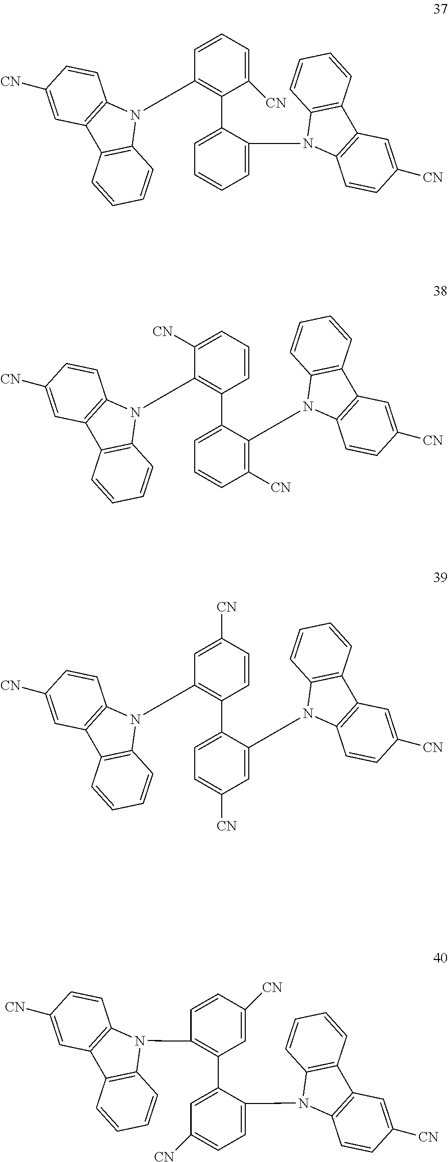

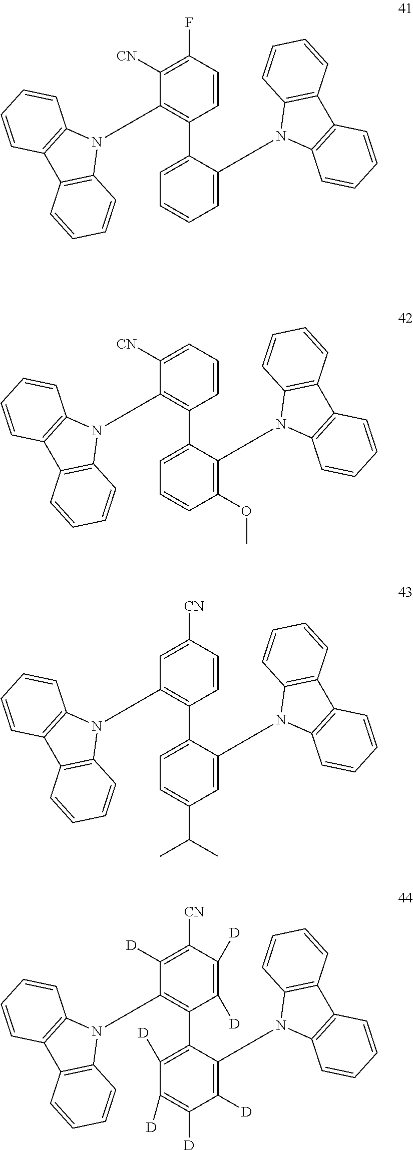

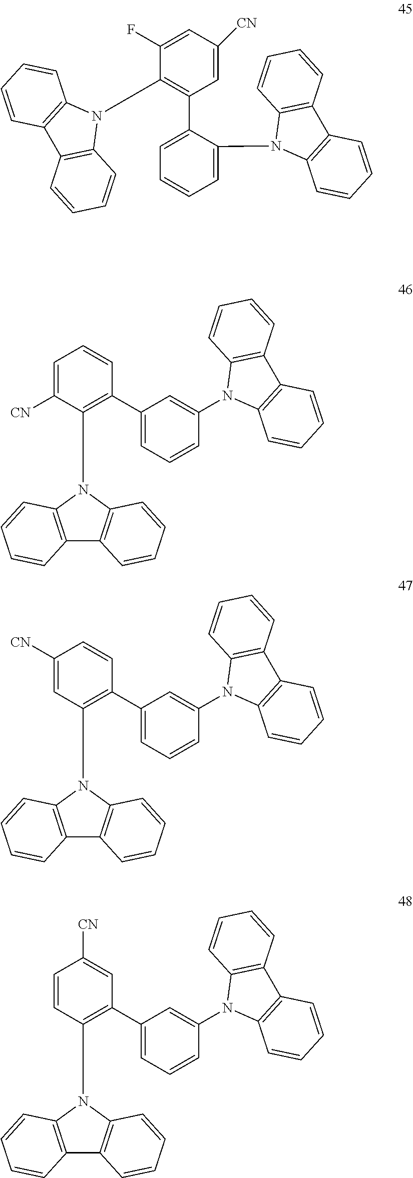

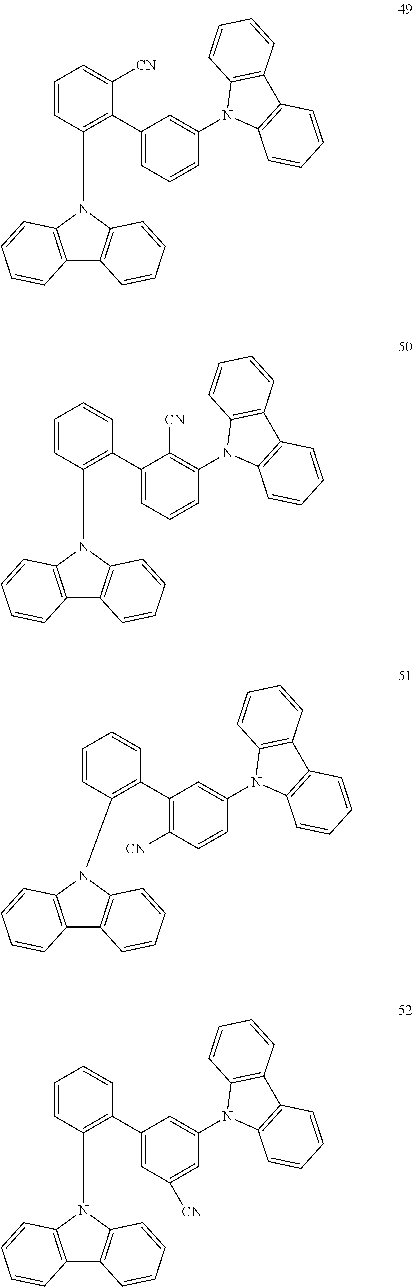

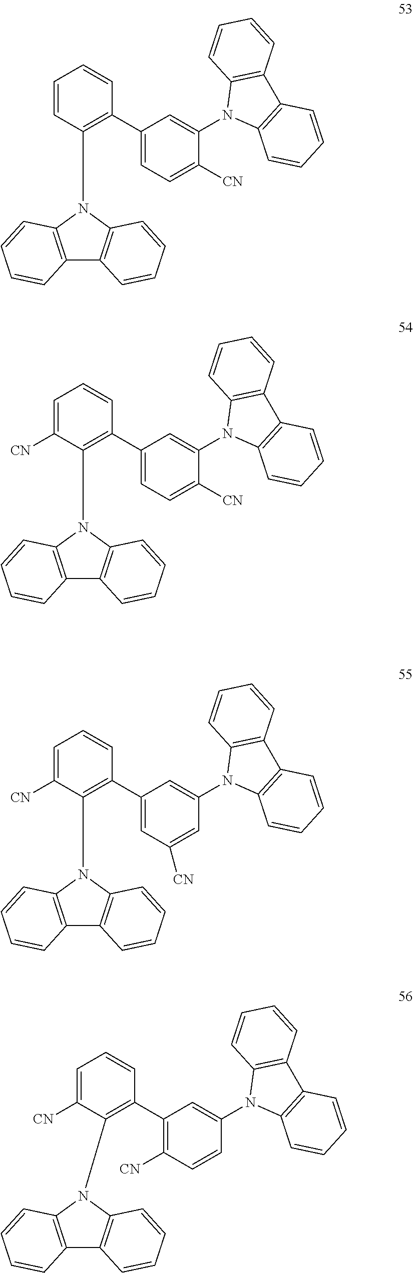

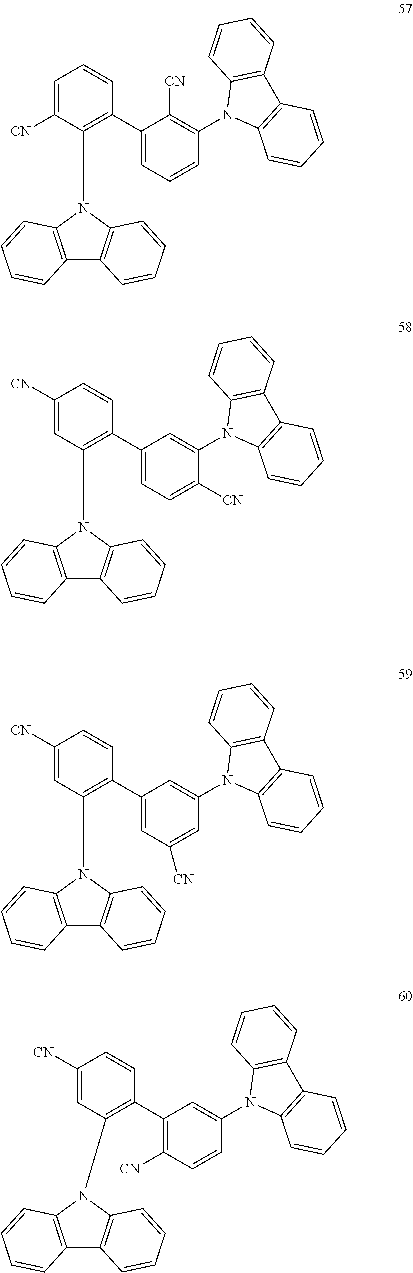

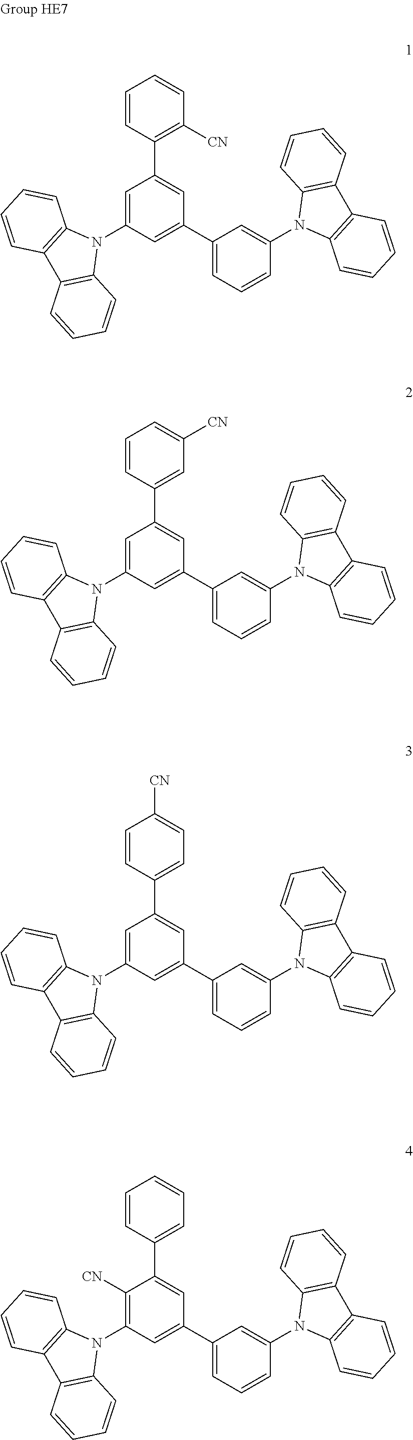

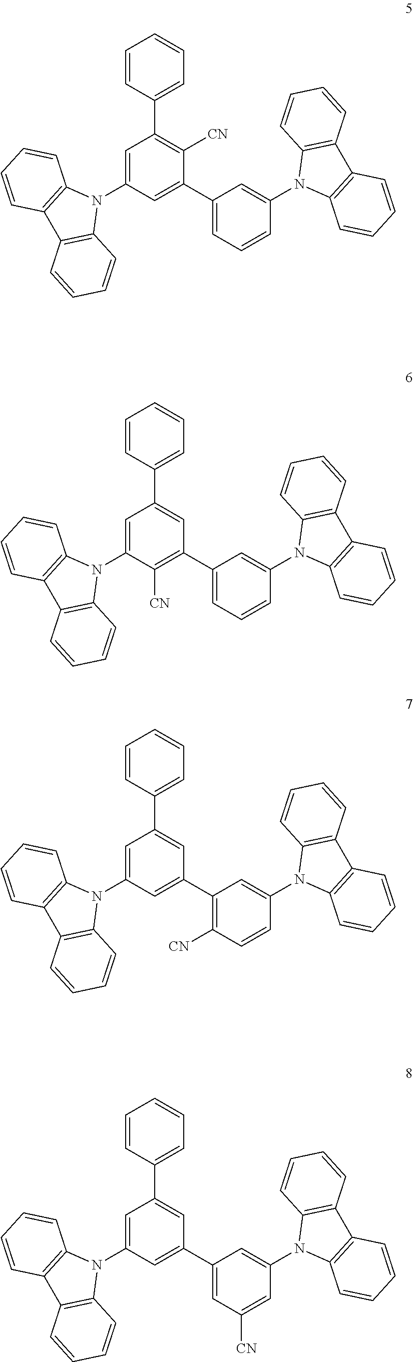

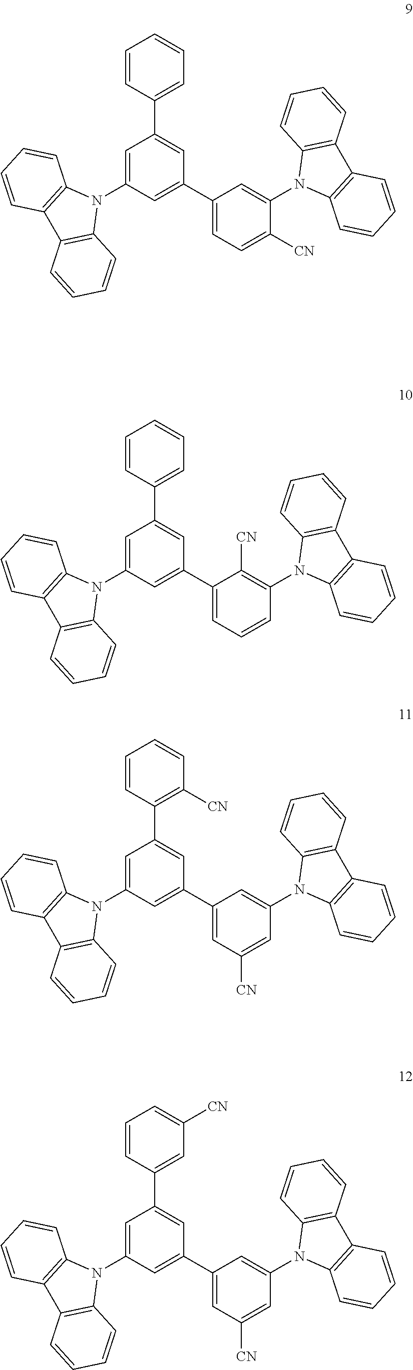 [SMILES: N#Cc1ccc(-c2cc(-c3ccccc3)cc(-n3c4ccccc4c4ccccc43)c2)cc1-n1c2ccccc2c2ccccc21 ; N#Cc1c(-c2cc(-c3ccccc3)cc(-n3c4ccccc4c4ccccc43)c2)cccc1-n1c2ccccc2c2ccccc21 ; N#Cc1cc(-c2cc(-c3ccccc3C#N)cc(-n3c4ccccc4c4ccccc43)c2)cc(-n2c3ccccc3c3ccccc32)c1 ; N#Cc1cccc(-c2cc(-c3cc(C#N)cc(-n4c5ccccc5c5ccccc54)c3)cc(-n3c4ccccc4c4ccccc43)c2)c1]

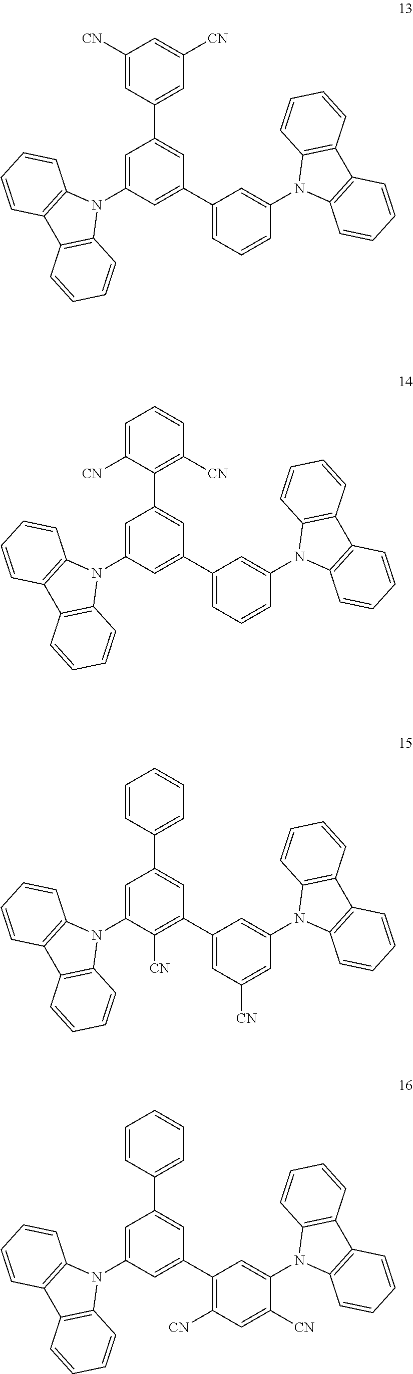

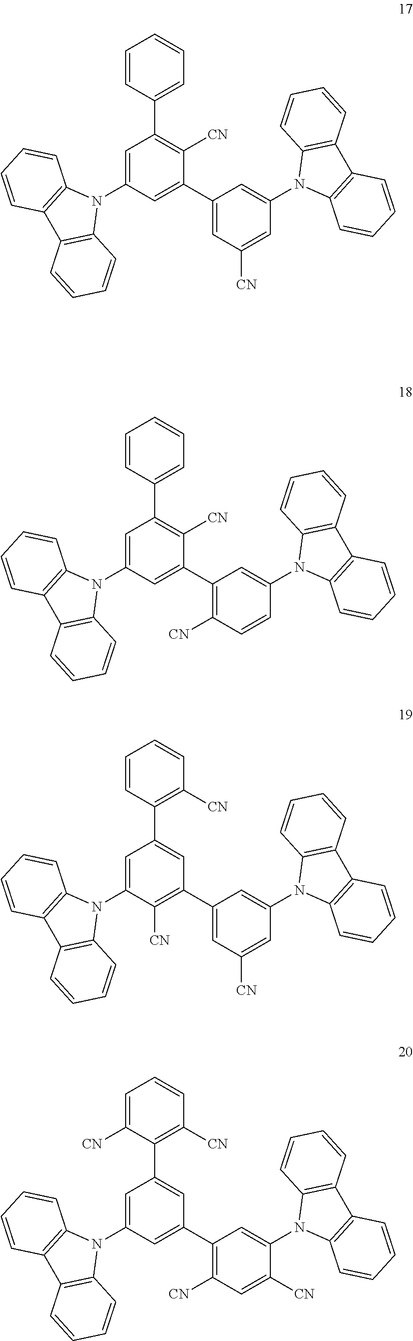

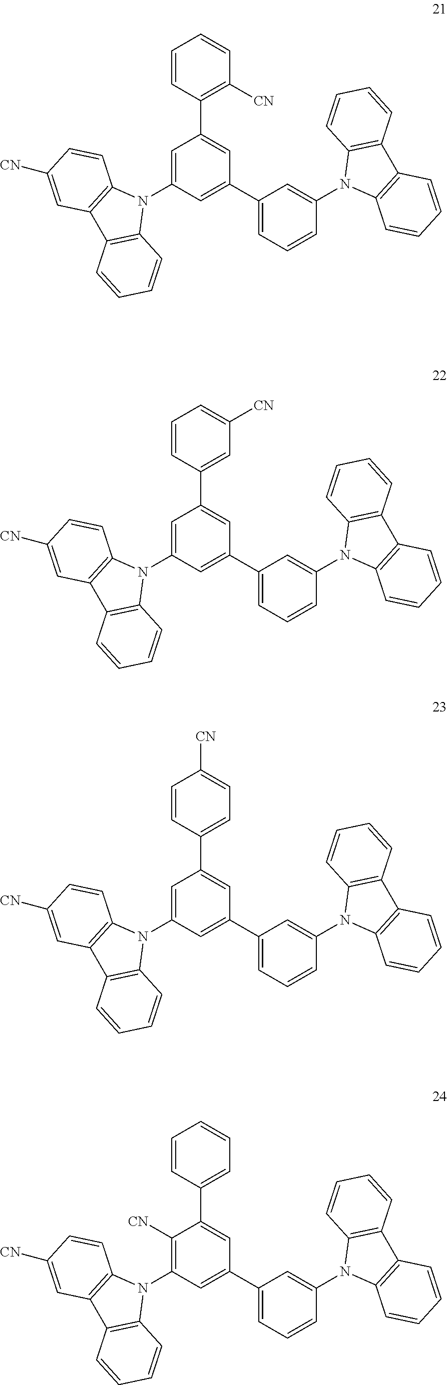

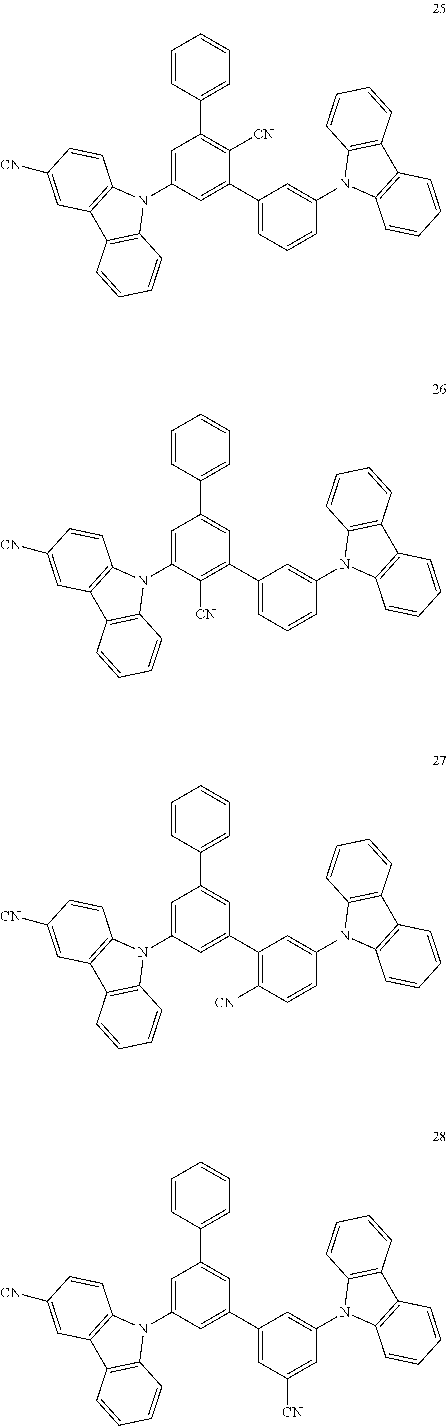

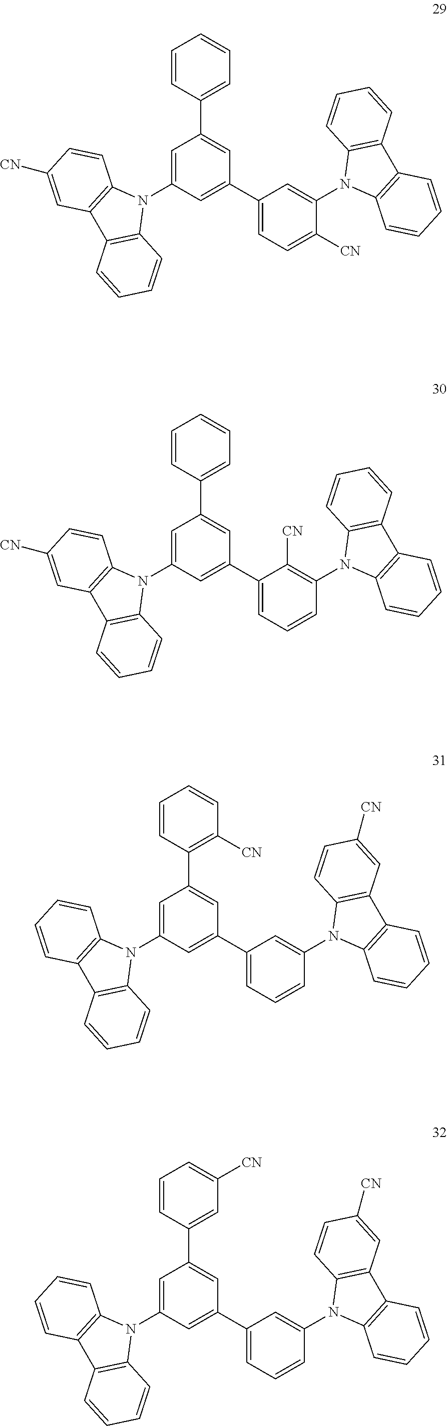

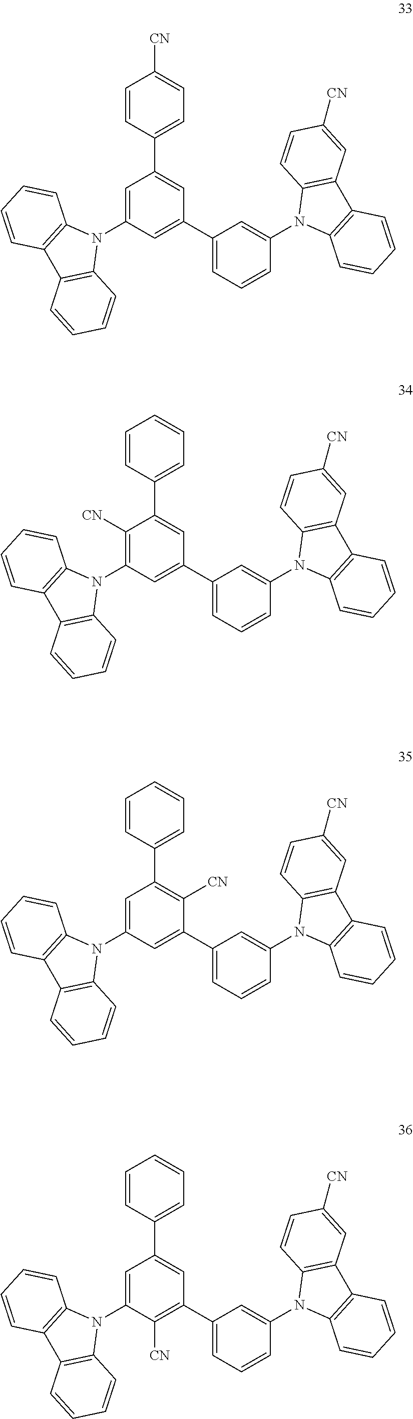

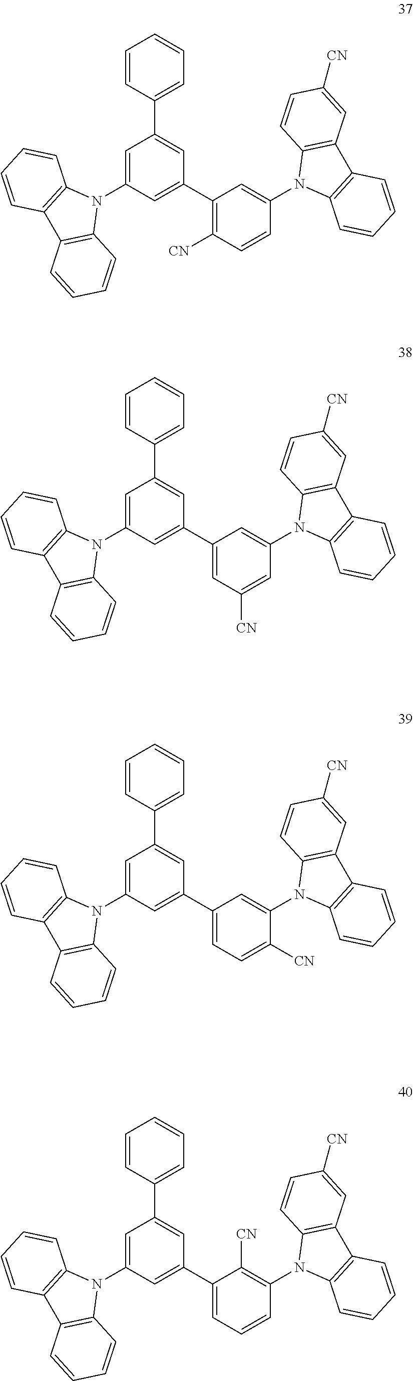

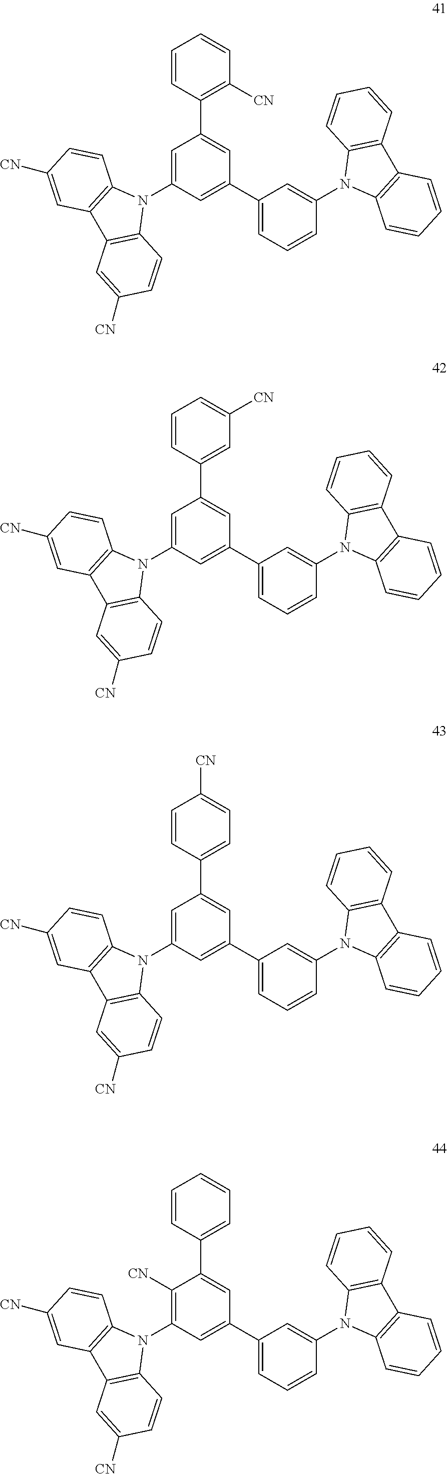

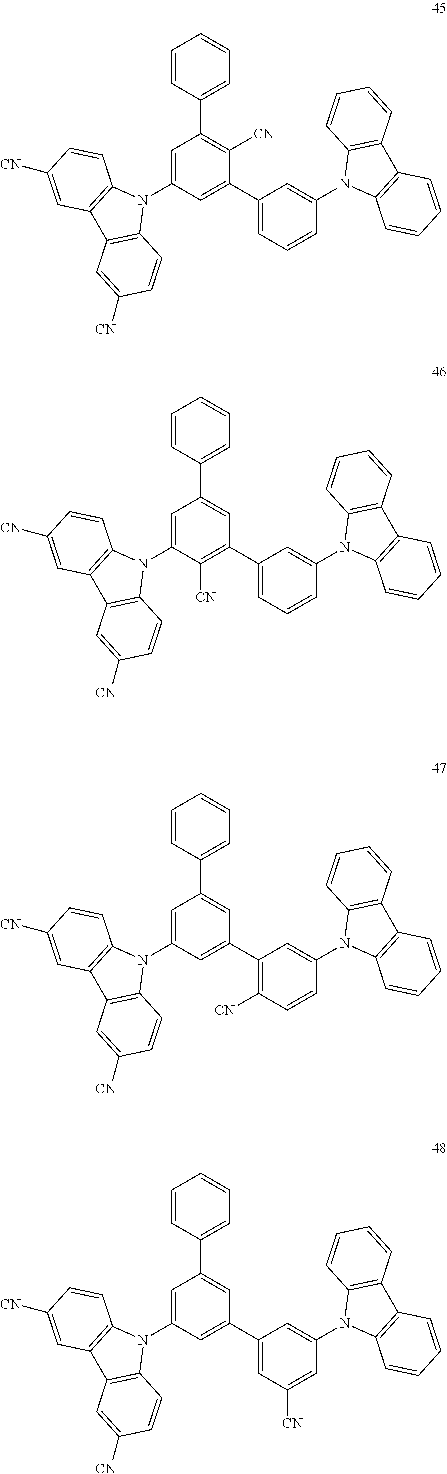

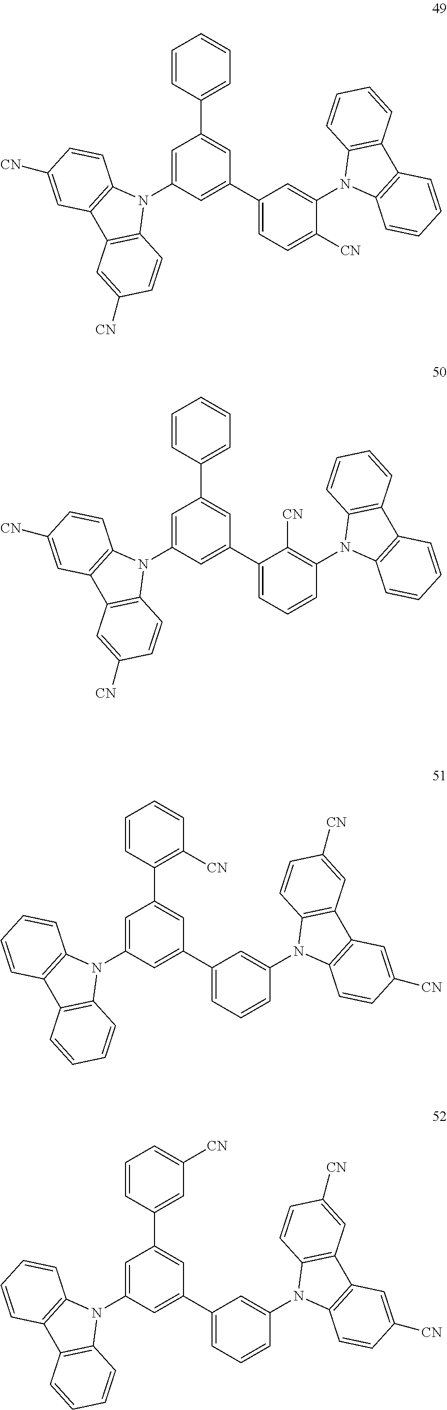

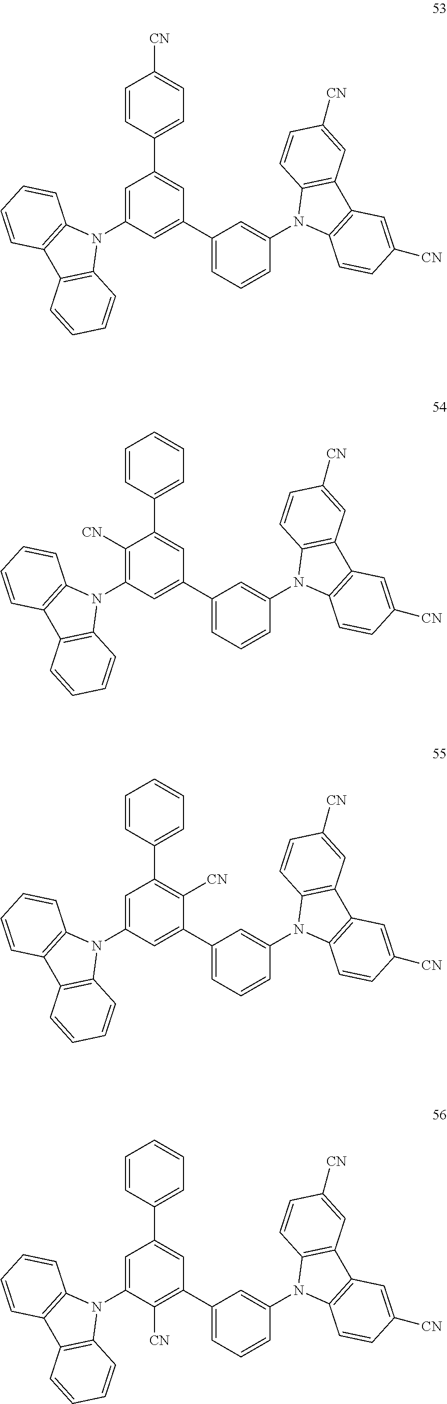

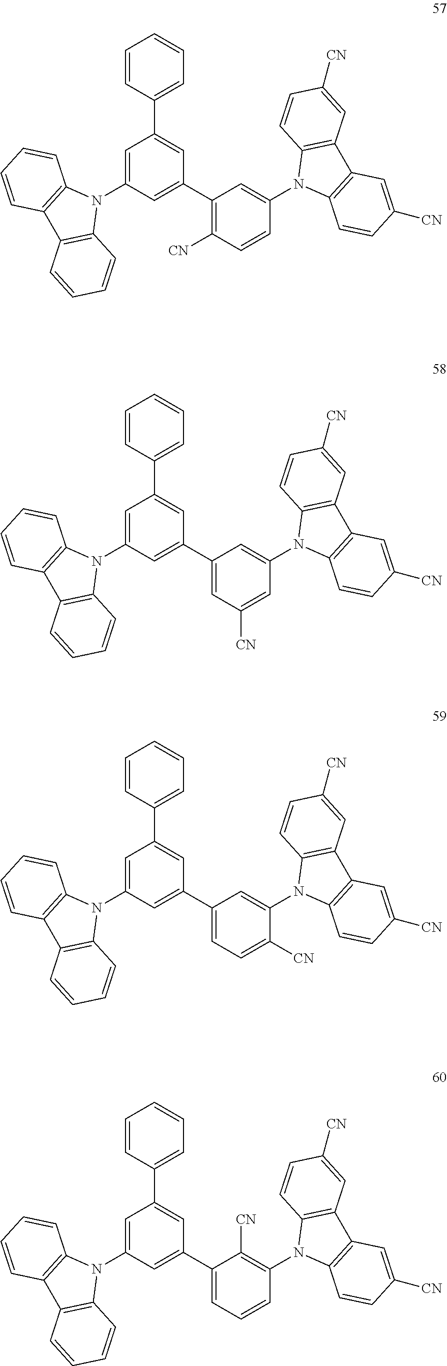

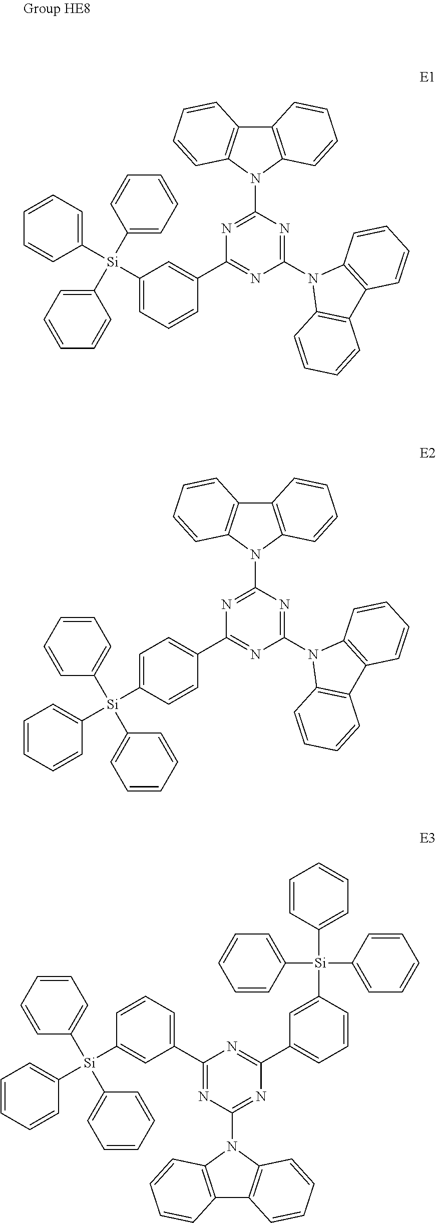 [SMILES: c1ccc([Si](c2ccccc2)(c2ccccc2)c2cccc(-c3nc(-n4c5ccccc5c5ccccc54)nc(-n4c5ccccc5c5ccccc54)n3)c2)cc1 ; c1ccc([Si](c2ccccc2)(c2ccccc2)c2ccc(-c3nc(-n4c5ccccc5c5ccccc54)nc(-n4c5ccccc5c5ccccc54)n3)cc2)cc1 ; c1ccc([Si](c2ccccc2)(c2ccccc2)c2cccc(-c3nc(-c4cccc([Si](c5ccccc5)(c5ccccc5)c5ccccc5)c4)nc(-n4c5ccccc5c5ccccc54)n3)c2)cc1]

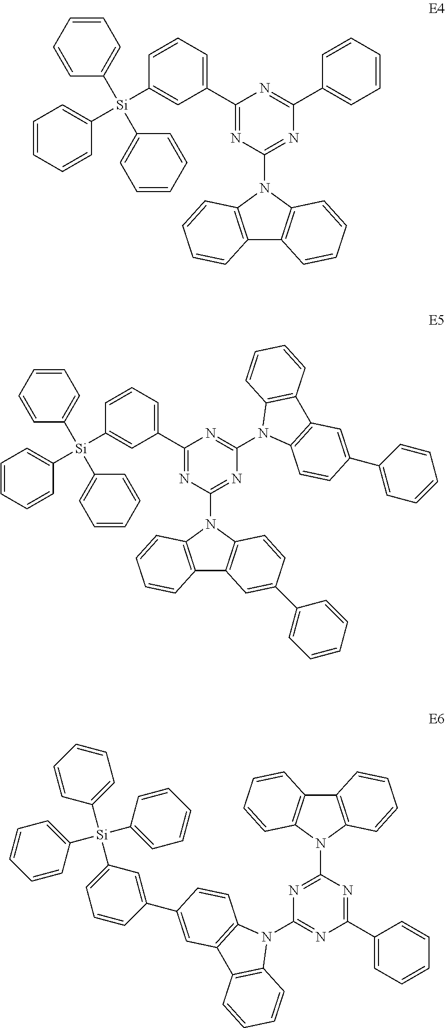 [SMILES: c1ccc(-c2nc(-c3cccc([Si](c4ccccc4)(c4ccccc4)c4ccccc4)c3)nc(-n3c4ccccc4c4ccccc43)n2)cc1 ; c1ccc(-c2ccc3c(c2)c2ccccc2n3-c2nc(-c3cccc([Si](c4ccccc4)(c4ccccc4)c4ccccc4)c3)nc(-n3c4ccccc4c4cc(-c5ccccc5)ccc43)n2)cc1 ; c1ccc(-c2nc(-n3c4ccccc4c4ccccc43)nc(-n3c4ccccc4c4cc(-c5cccc([Si](c6ccccc6)(c6ccccc6)c6ccccc6)c5)ccc43)n2)cc1]

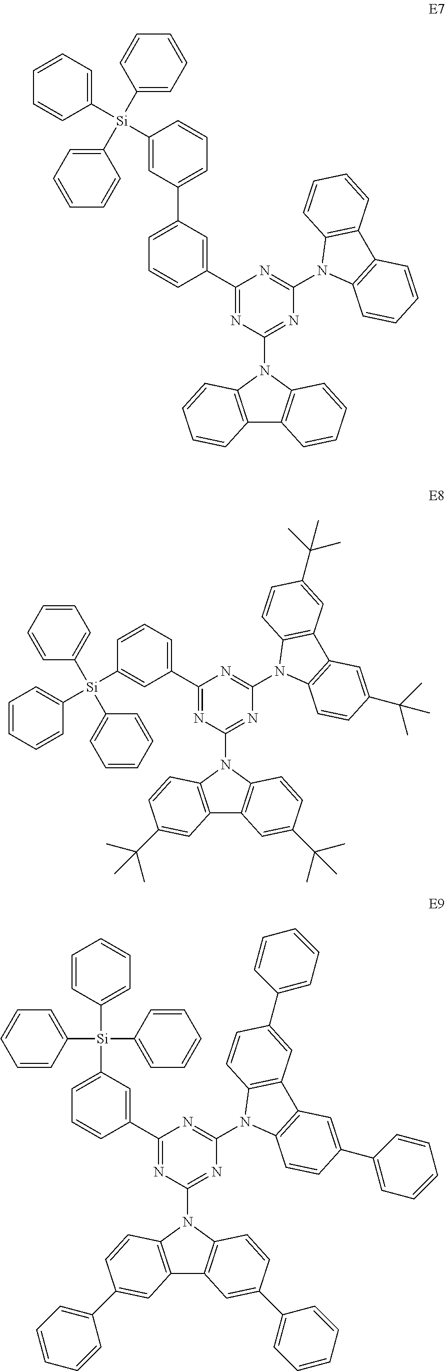 [SMILES: c1ccc([Si](c2ccccc2)(c2ccccc2)c2cccc(-c3cccc(-c4nc(-n5c6ccccc6c6ccccc65)nc(-n5c6ccccc6c6ccccc65)n4)c3)c2)cc1 ; CC(C)(C)c1ccc2c(c1)c1cc(C(C)(C)C)ccc1n2-c1nc(-c2cccc([Si](c3ccccc3)(c3ccccc3)c3ccccc3)c2)nc(-n2c3ccc(C(C)(C)C)cc3c3cc(C(C)(C)C)ccc32)n1 ; c1ccc(-c2ccc3c(c2)c2cc(-c4ccccc4)ccc2n3-c2nc(-c3cccc([Si](c4ccccc4)(c4ccccc4)c4ccccc4)c3)nc(-n3c4ccc(-c5ccccc5)cc4c4cc(-c5ccccc5)ccc43)n2)cc1]

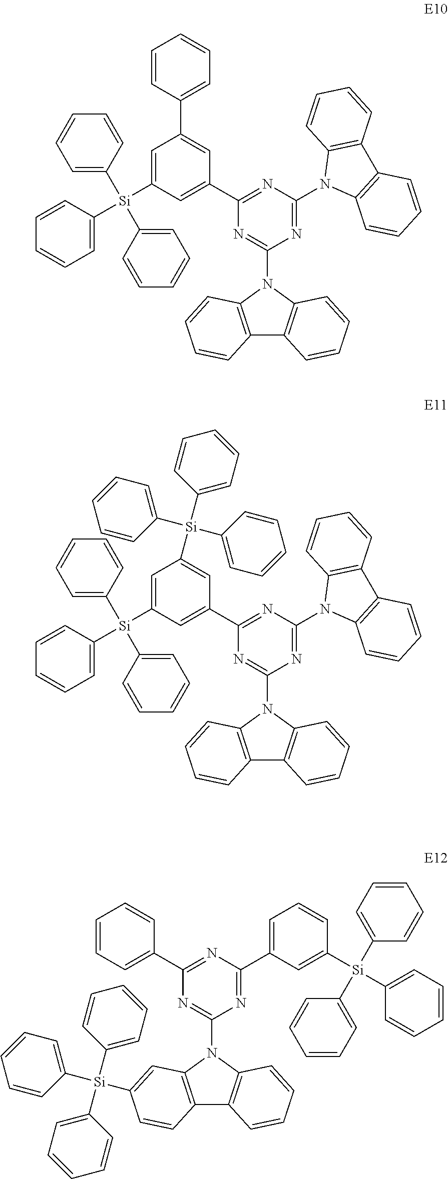 [SMILES: c1ccc(-c2cc(-c3nc(-n4c5ccccc5c5ccccc54)nc(-n4c5ccccc5c5ccccc54)n3)cc([Si](c3ccccc3)(c3ccccc3)c3ccccc3)c2)cc1 ; c1ccc([Si](c2ccccc2)(c2ccccc2)c2cc(-c3nc(-n4c5ccccc5c5ccccc54)nc(-n4c5ccccc5c5ccccc54)n3)cc([Si](c3ccccc3)(c3ccccc3)c3ccccc3)c2)cc1 ; c1ccc(-c2nc(-c3cccc([Si](c4ccccc4)(c4ccccc4)c4ccccc4)c3)nc(-n3c4ccccc4c4ccc([Si](c5ccccc5)(c5ccccc5)c5ccccc5)cc43)n2)cc1]

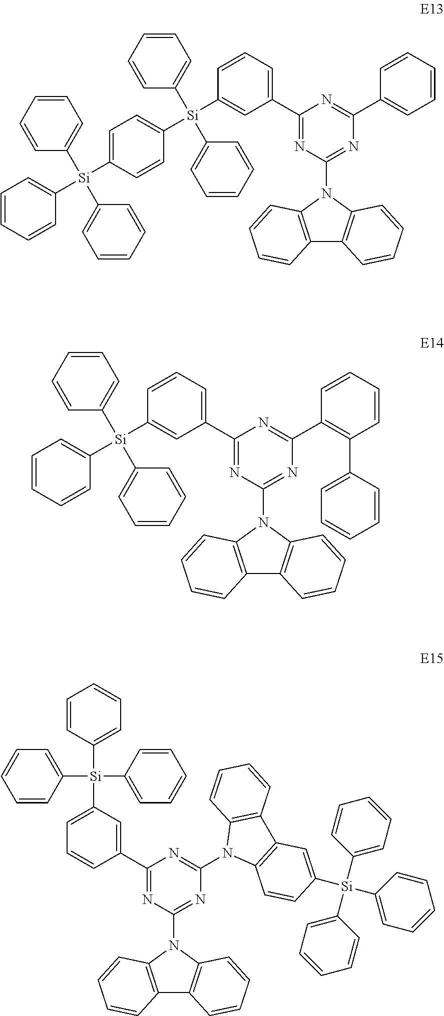 [SMILES: c1ccc(-c2nc(-c3cccc([Si](c4ccccc4)(c4ccccc4)c4ccc([Si](c5ccccc5)(c5ccccc5)c5ccccc5)cc4)c3)nc(-n3c4ccccc4c4ccccc43)n2)cc1 ; c1ccc(-c2ccccc2-c2nc(-c3cccc([Si](c4ccccc4)(c4ccccc4)c4ccccc4)c3)nc(-n3c4ccccc4c4ccccc43)n2)cc1 ; c1ccc([Si](c2ccccc2)(c2ccccc2)c2cccc(-c3nc(-n4c5ccccc5c5ccccc54)nc(-n4c5ccccc5c5cc([Si](c6ccccc6)(c6ccccc6)c6ccccc6)ccc54)n3)c2)cc1]

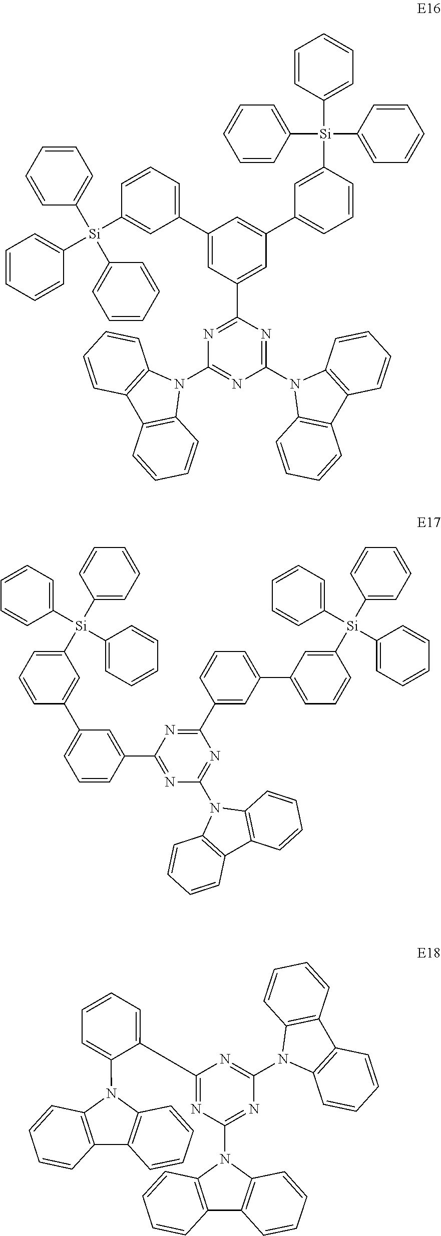 [SMILES: c1ccc([Si](c2ccccc2)(c2ccccc2)c2cccc(-c3cc(-c4cccc([Si](c5ccccc5)(c5ccccc5)c5ccccc5)c4)cc(-c4nc(-n5c6ccccc6c6ccccc65)nc(-n5c6ccccc6c6ccccc65)n4)c3)c2)cc1 ; c1ccc([Si](c2ccccc2)(c2ccccc2)c2cccc(-c3cccc(-c4nc(-c5cccc(-c6cccc([Si](c7ccccc7)(c7ccccc7)c7ccccc7)c6)c5)nc(-n5c6ccccc6c6ccccc65)n4)c3)c2)cc1 ; c1ccc(-n2c3ccccc3c3ccccc32)c(-c2nc(-n3c4ccccc4c4ccccc43)nc(-n3c4ccccc4c4ccccc43)n2)c1]

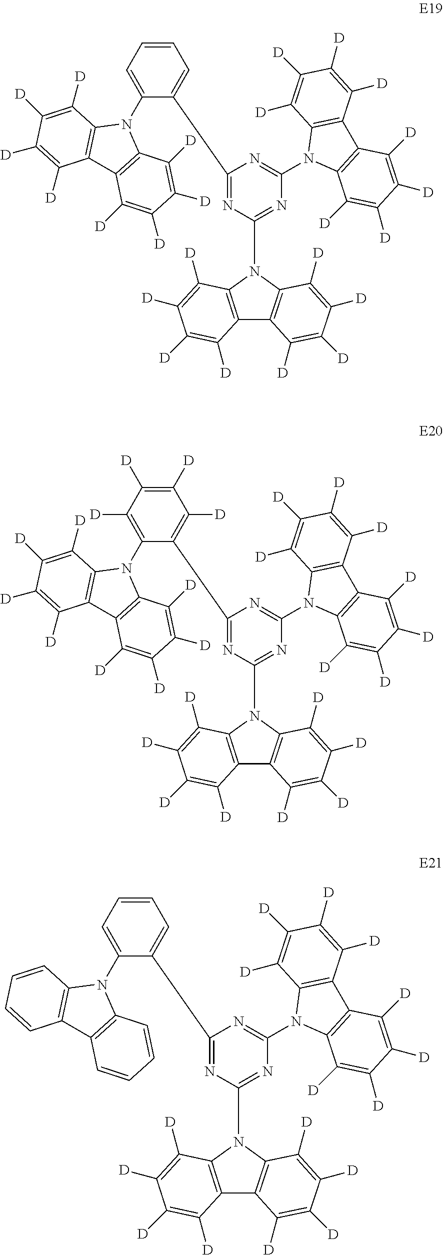

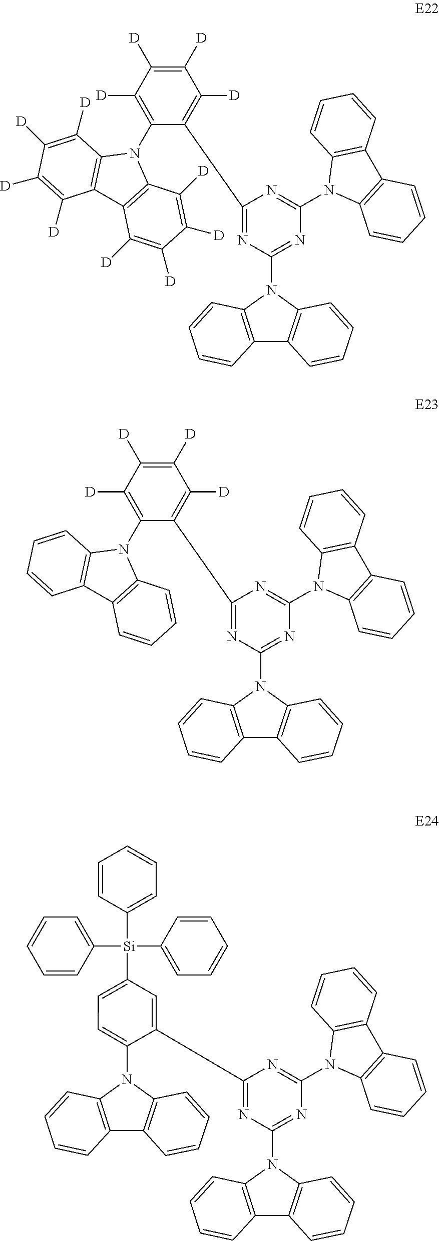 [SMILES: [2H]c1c([2H])c([2H])c(-n2c3c([2H])c([2H])c([2H])c([2H])c3c3c([2H])c([2H])c([2H])c([2H])c32)c(-c2nc(-n3c4ccccc4c4ccccc43)nc(-n3c4ccccc4c4ccccc43)n2)c1[2H] ; [2H]c1c([2H])c([2H])c(-n2c3ccccc3c3ccccc32)c(-c2nc(-n3c4ccccc4c4ccccc43)nc(-n3c4ccccc4c4ccccc43)n2)c1[2H] ; c1ccc([Si](c2ccccc2)(c2ccccc2)c2ccc(-n3c4ccccc4c4ccccc43)c(-c3nc(-n4c5ccccc5c5ccccc54)nc(-n4c5ccccc5c5ccccc54)n3)c2)cc1]

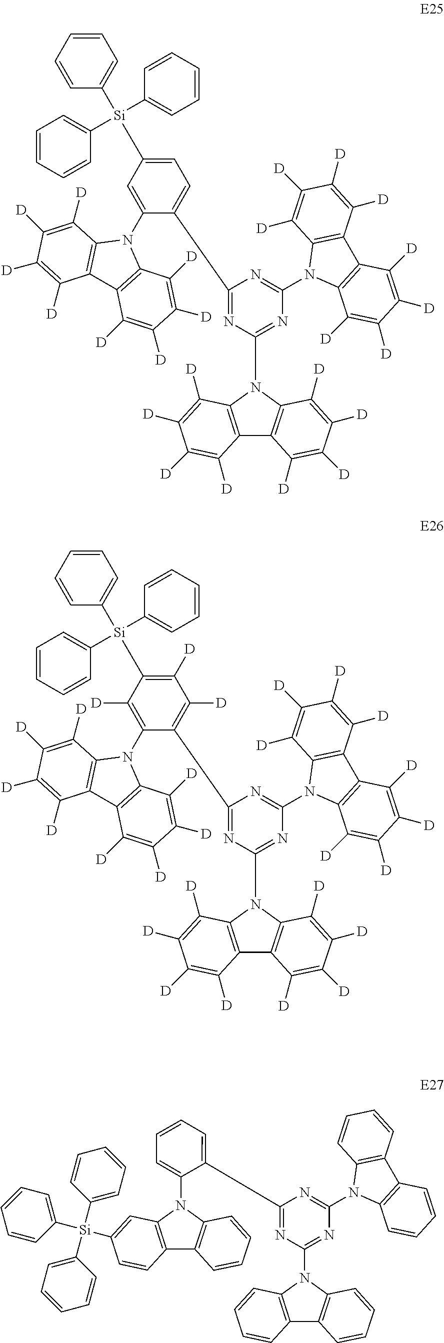

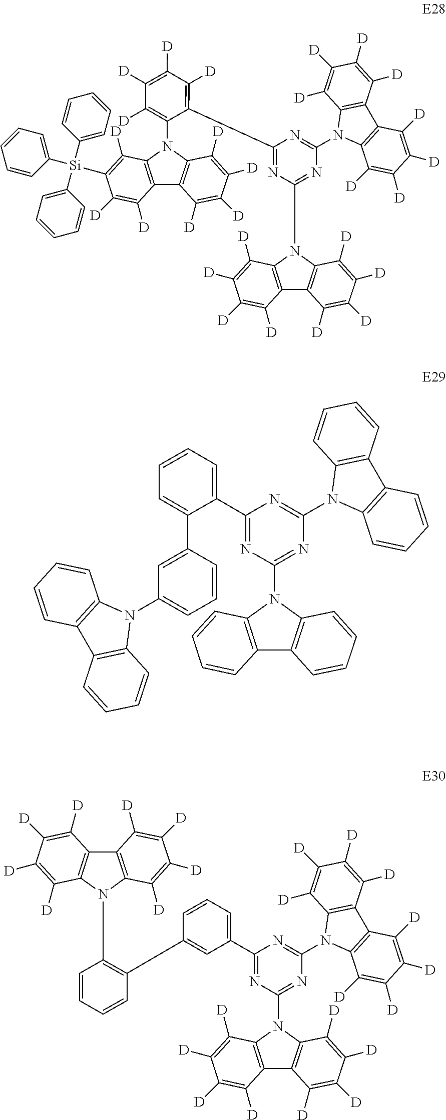

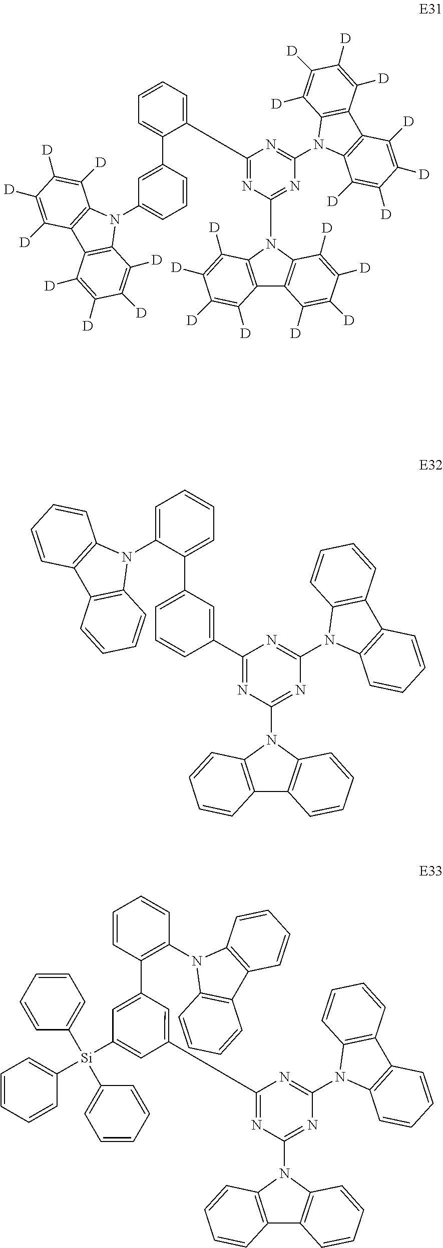 [SMILES: [2H]c1c([2H])c([2H])c2c(c1[2H])c1c([2H])c([2H])c([2H])c([2H])c1n2-c1cccc(-c2ccccc2-c2nc(-n3c4c([2H])c([2H])c([2H])c([2H])c4c4c([2H])c([2H])c([2H])c([2H])c43)nc(-n3c4c([2H])c([2H])c([2H])c([2H])c4c4c([2H])c([2H])c([2H])c([2H])c43)n2)c1 ; c1cc(-c2nc(-n3c4ccccc4c4ccccc43)nc(-n3c4ccccc4c4ccccc43)n2)cc(-c2ccccc2-n2c3ccccc3c3ccccc32)c1 ; c1ccc([Si](c2ccccc2)(c2ccccc2)c2cc(-c3nc(-n4c5ccccc5c5ccccc54)nc(-n4c5ccccc5c5ccccc54)n3)cc(-c3ccccc3-n3c4ccccc4c4ccccc43)c2)cc1]

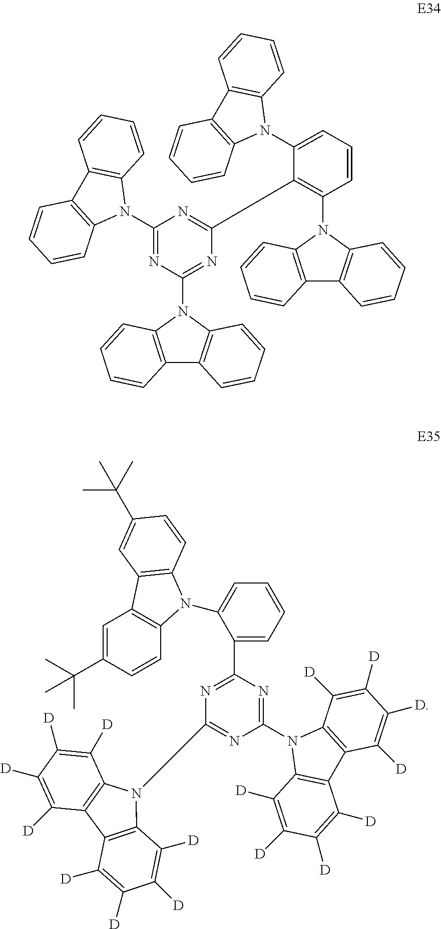

In one or more embodiments, the hole-transporting host may include at least one of Compounds H-H1 to H-H103, but embodiments are not limited thereto:

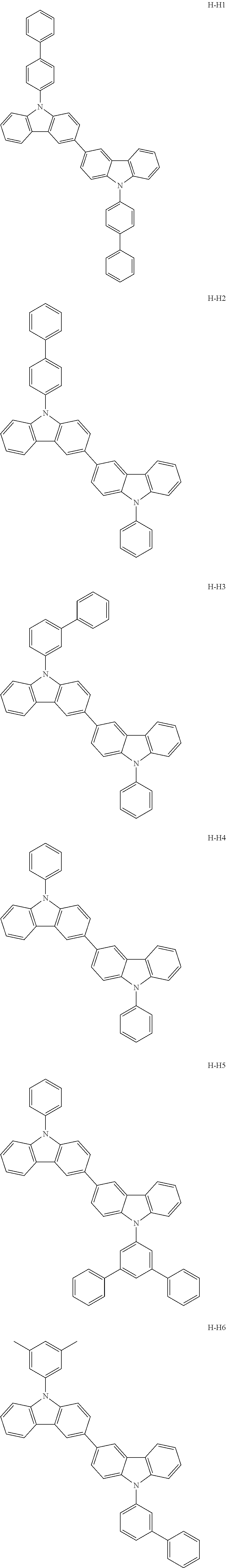 [SMILES: c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-c2ccc(-n3c4ccccc4c4cc(-c5ccc6c(c5)c5ccccc5n6-c5ccccc5)ccc43)cc2)cc1 ; c1ccc(-c2cccc(-n3c4ccccc4c4cc(-c5ccc6c(c5)c5ccccc5n6-c5ccccc5)ccc43)c2)cc1 ; c1ccc(-n2c3ccccc3c3cc(-c4ccc5c(c4)c4ccccc4n5-c4ccccc4)ccc32)cc1 ; c1ccc(-c2cc(-c3ccccc3)cc(-n3c4ccccc4c4cc(-c5ccc6c(c5)c5ccccc5n6-c5ccccc5)ccc43)c2)cc1 ; Cc1cc(C)cc(-n2c3ccccc3c3cc(-c4ccc5c(c4)c4ccccc4n5-c4cccc(-c5ccccc5)c4)ccc32)c1]

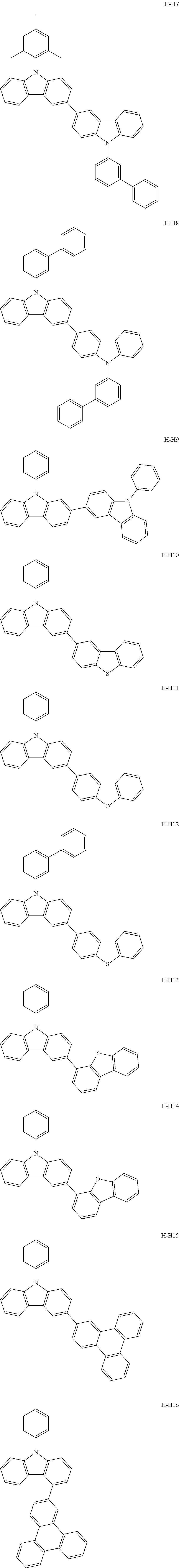

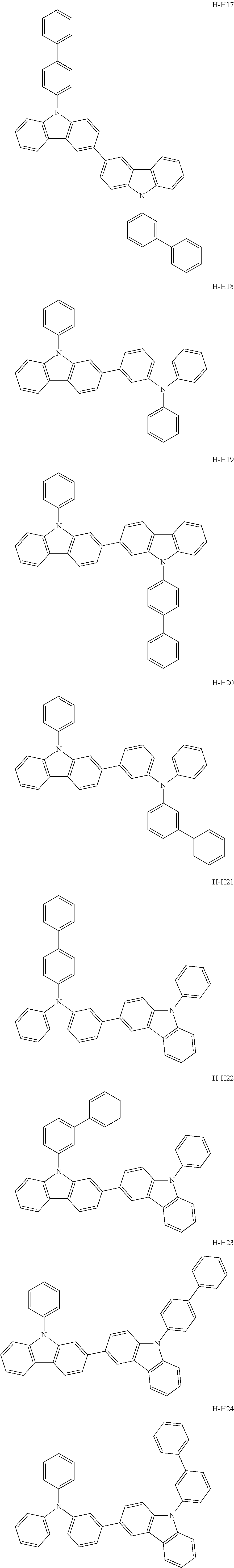

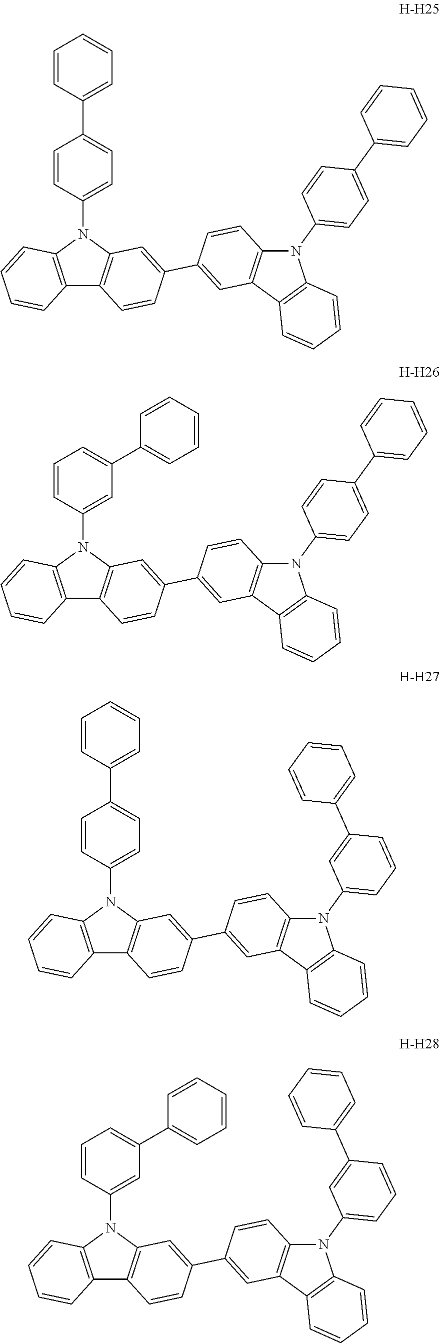 [SMILES: c1ccc(-c2ccc(-n3c4ccccc4c4cc(-c5ccc6c7ccccc7n(-c7ccc(-c8ccccc8)cc7)c6c5)ccc43)cc2)cc1 ; c1ccc(-c2ccc(-n3c4ccccc4c4cc(-c5ccc6c7ccccc7n(-c7cccc(-c8ccccc8)c7)c6c5)ccc43)cc2)cc1 ; c1ccc(-c2ccc(-n3c4ccccc4c4ccc(-c5ccc6c(c5)c5ccccc5n6-c5cccc(-c6ccccc6)c5)cc43)cc2)cc1 ; c1ccc(-c2cccc(-n3c4ccccc4c4cc(-c5ccc6c7ccccc7n(-c7cccc(-c8ccccc8)c7)c6c5)ccc43)c2)cc1]

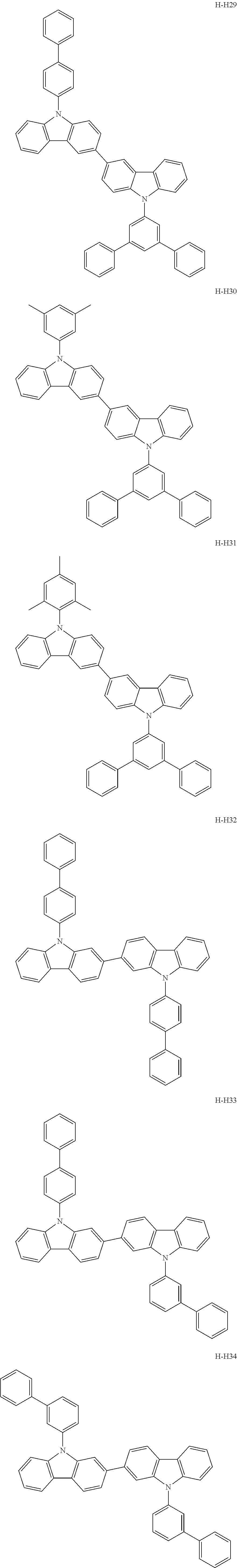

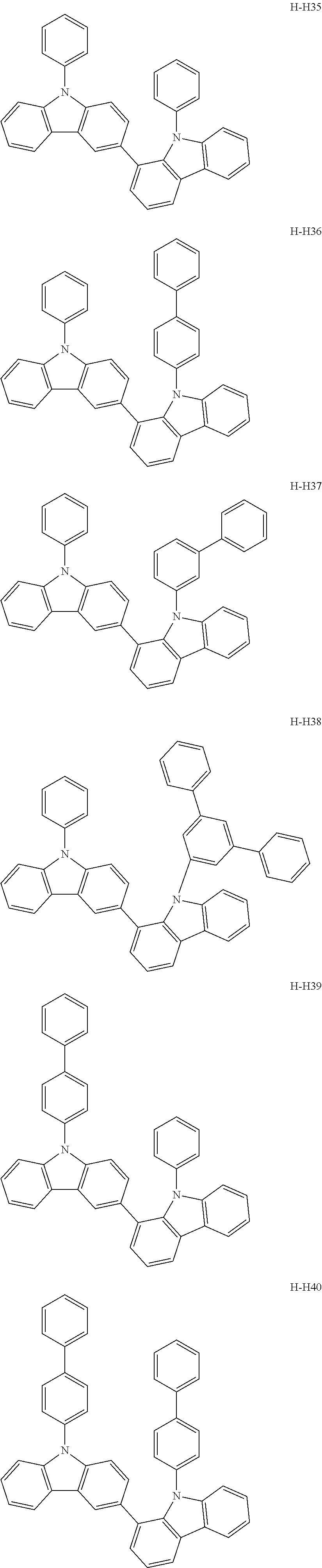

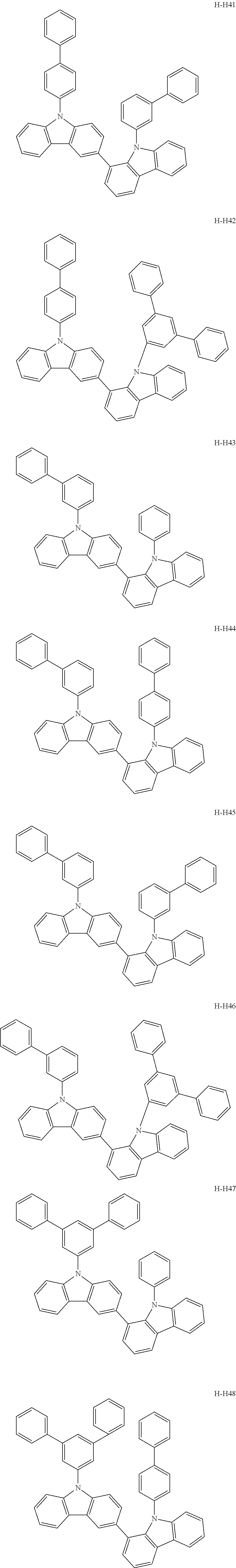

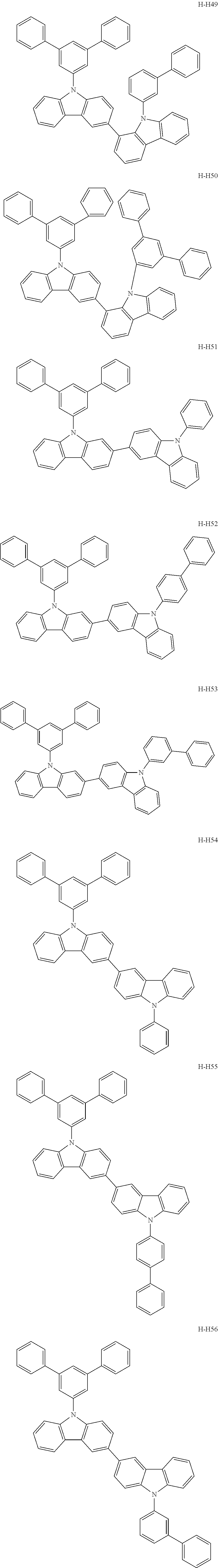

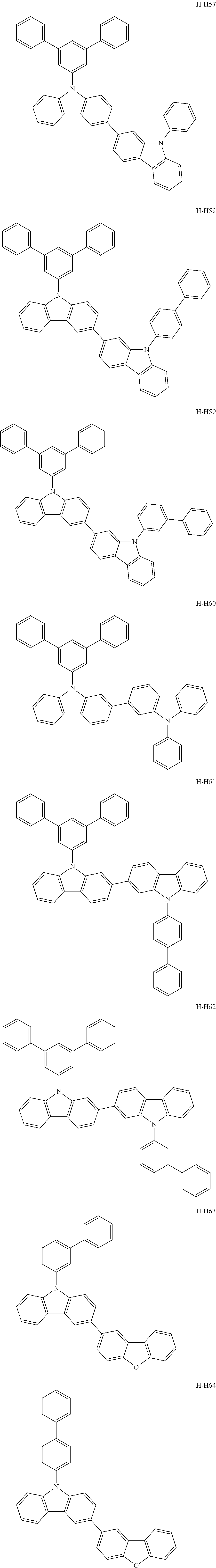

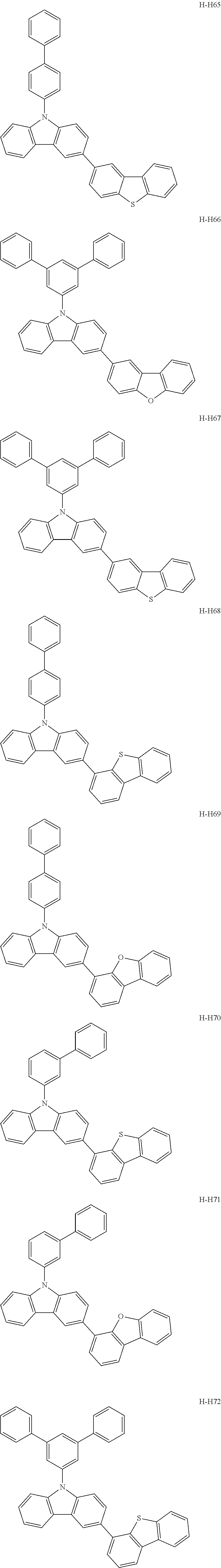

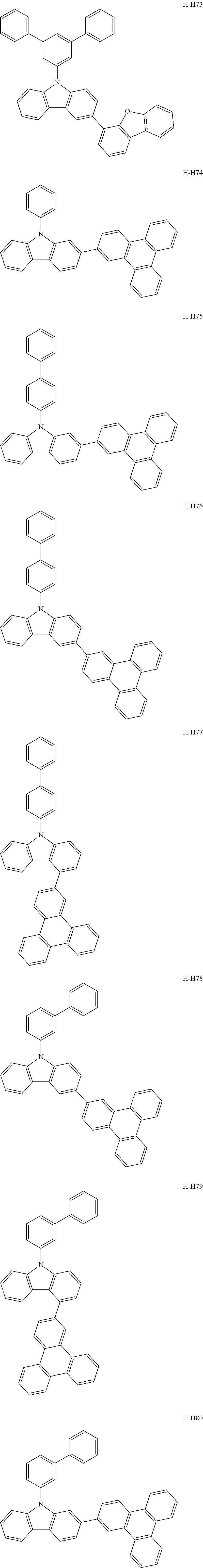

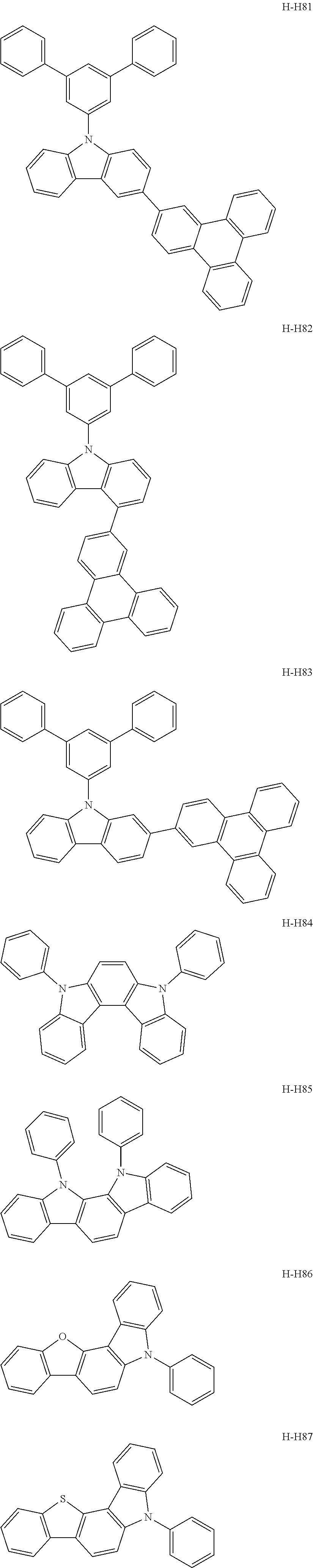

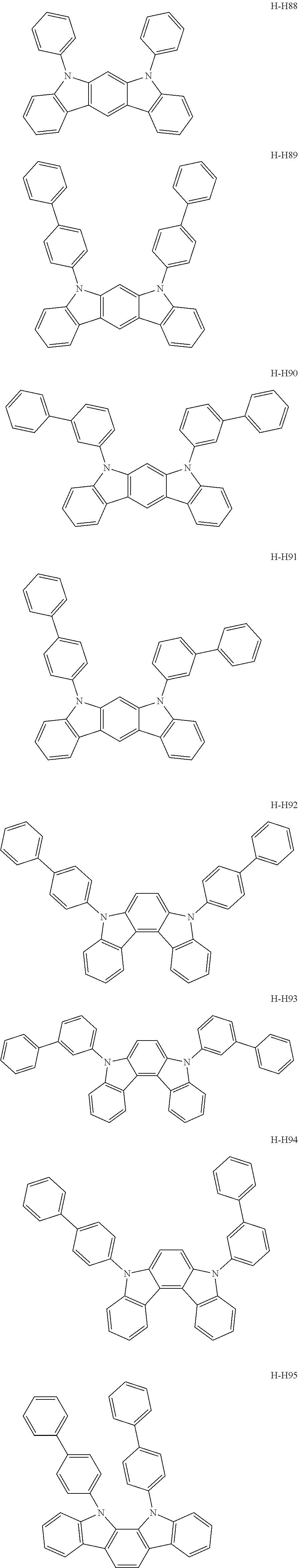

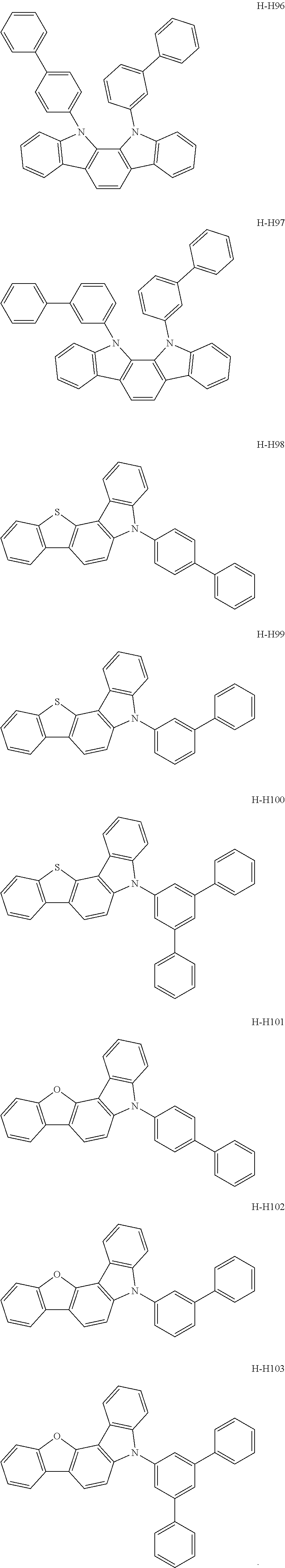

In one or more embodiments, the bipolar host may be a compound of Group HEH1, but embodiments are not limited thereto:

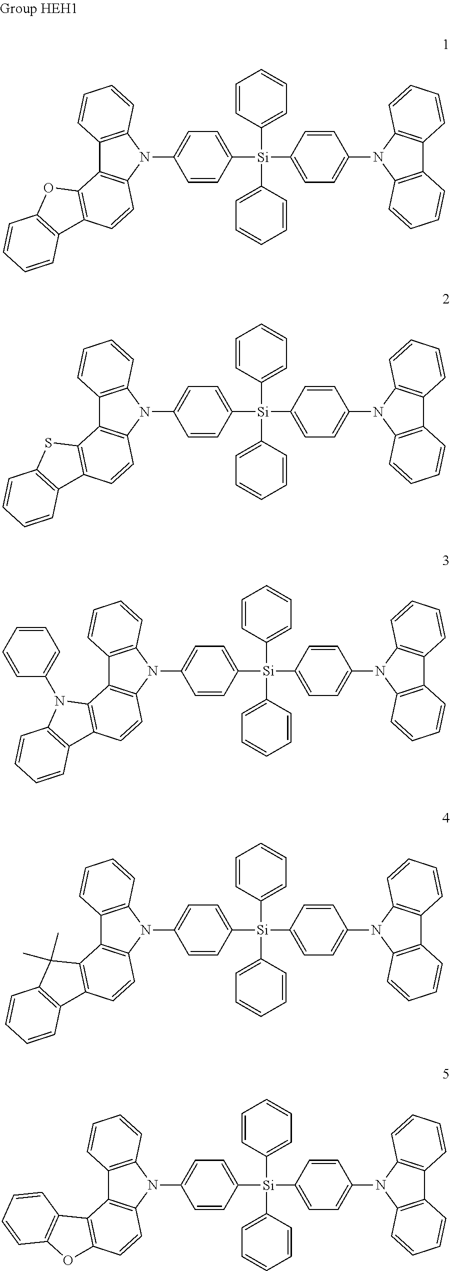

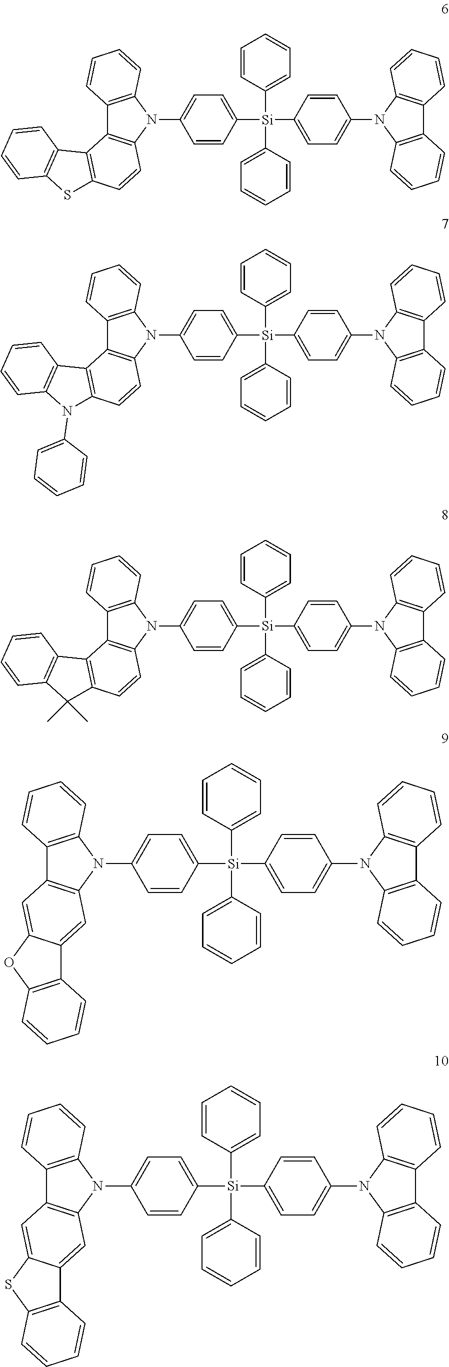

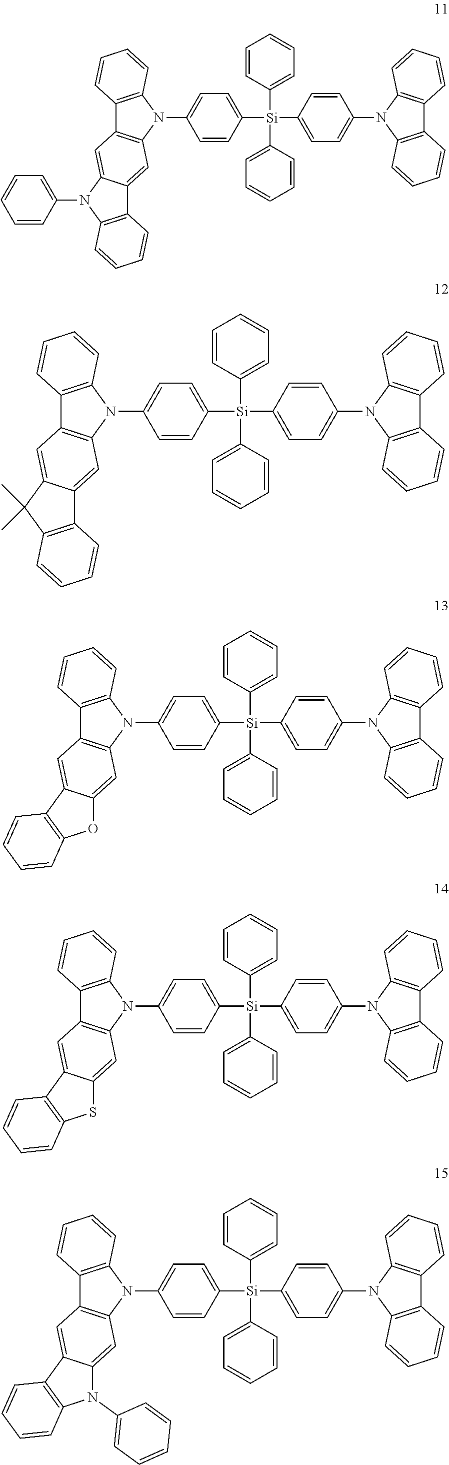

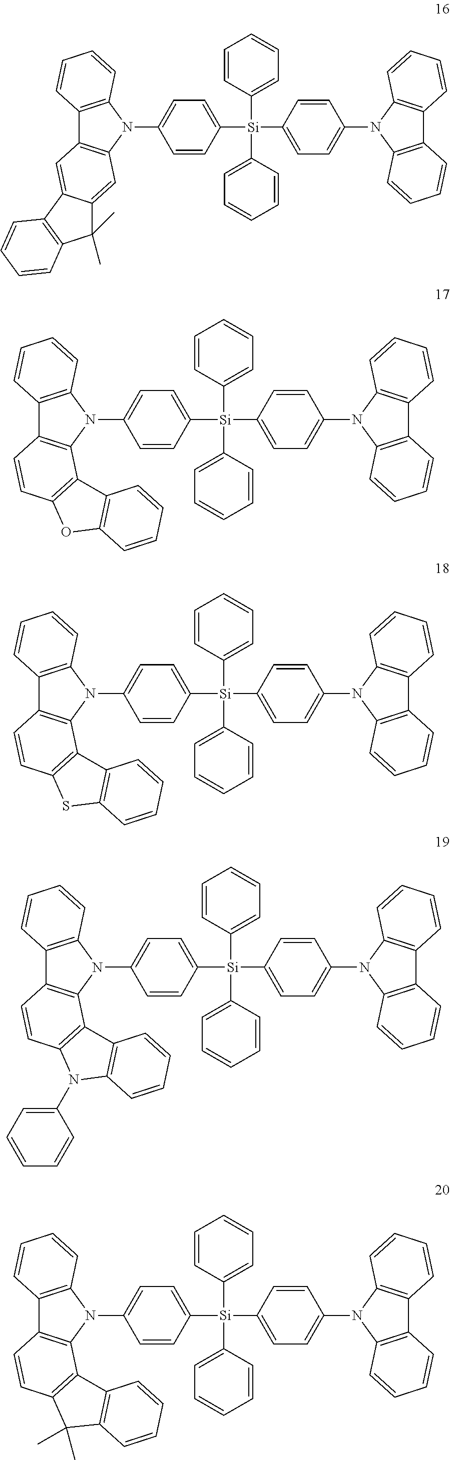

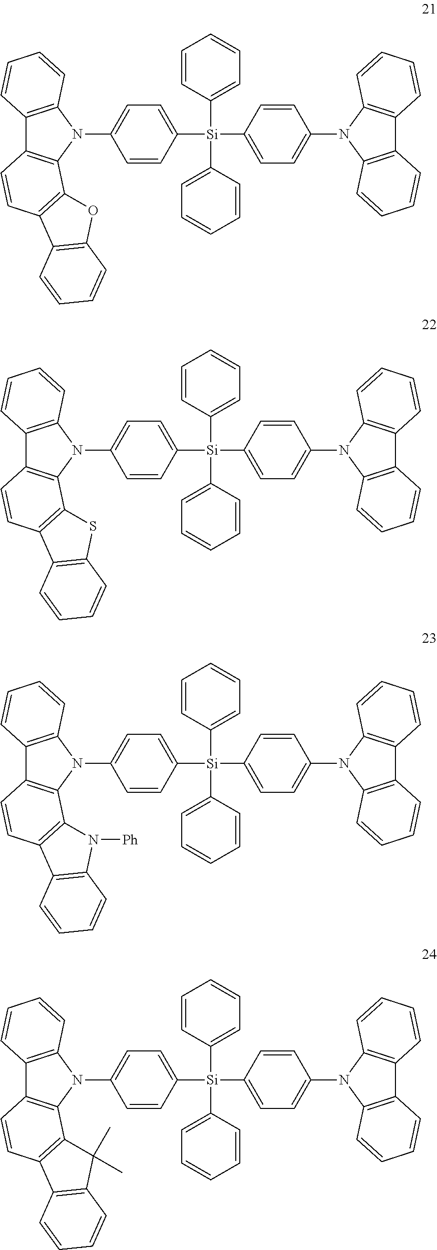 [SMILES: c1ccc([Si](c2ccccc2)(c2ccc(-n3c4ccccc4c4ccccc43)cc2)c2ccc(-n3c4ccccc4c4ccc5c6ccccc6oc5c43)cc2)cc1 ; c1ccc([Si](c2ccccc2)(c2ccc(-n3c4ccccc4c4ccccc43)cc2)c2ccc(-n3c4ccccc4c4ccc5c6ccccc6sc5c43)cc2)cc1 ; c1ccc(-n2c3ccccc3c3ccc4c5ccccc5n(-c5ccc([Si](c6ccccc6)(c6ccccc6)c6ccc(-n7c8ccccc8c8ccccc87)cc6)cc5)c4c32)cc1 ; CC1(C)c2ccccc2-c2ccc3c4ccccc4n(-c4ccc([Si](c5ccccc5)(c5ccccc5)c5ccc(-n6c7ccccc7c7ccccc76)cc5)cc4)c3c21]

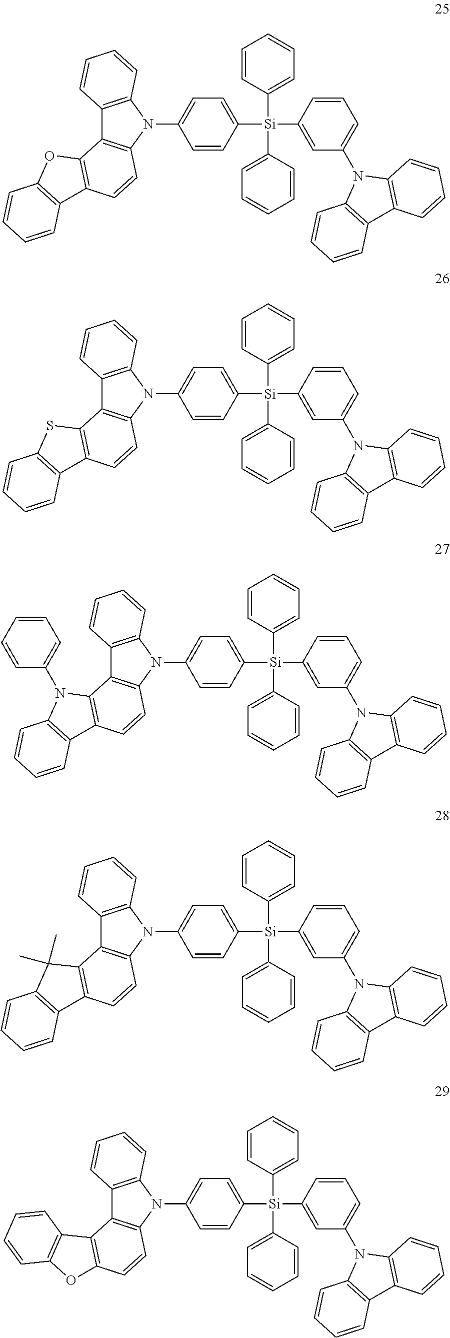

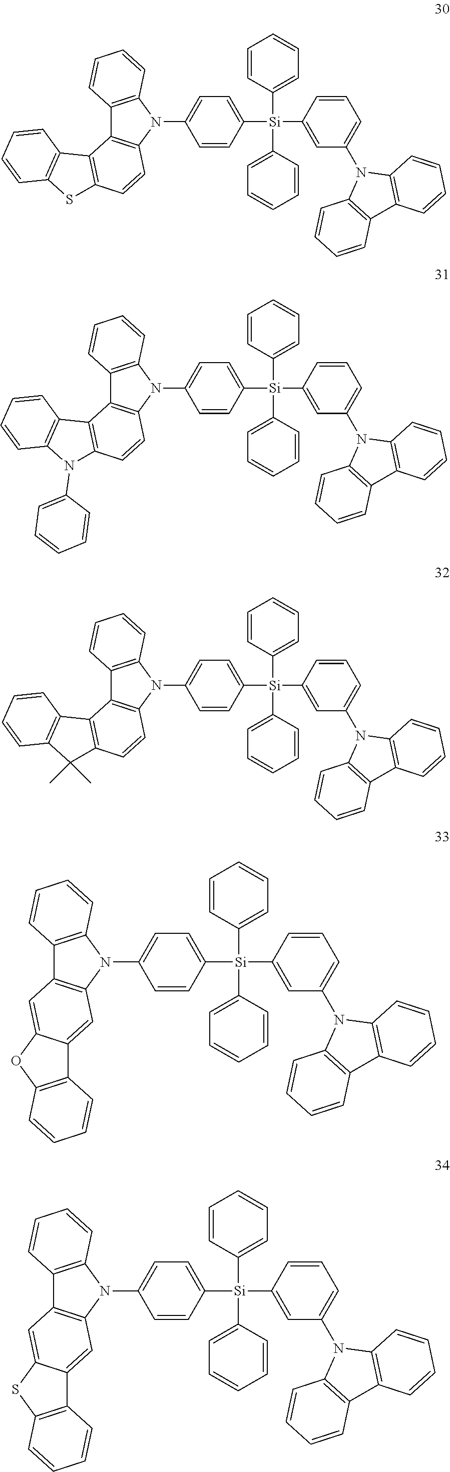

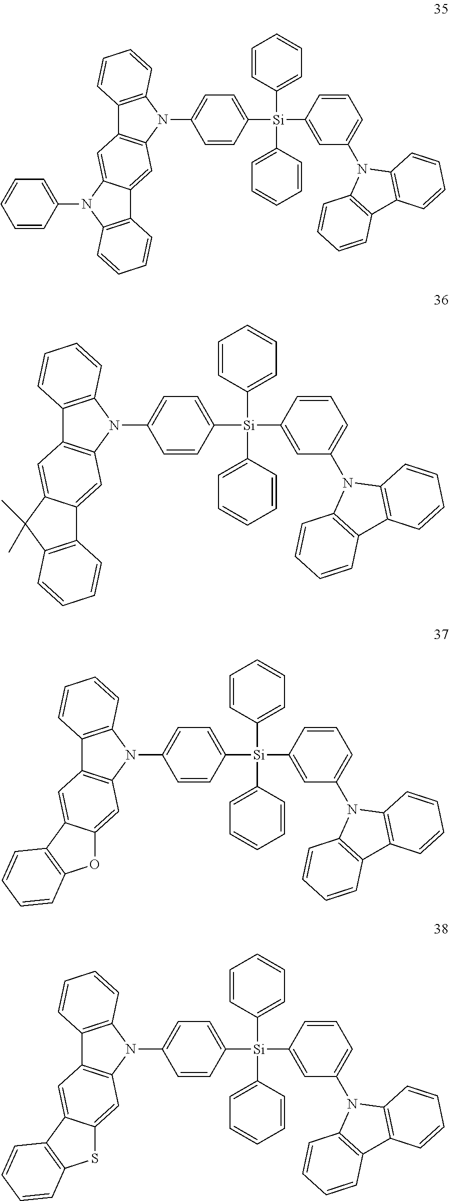 [SMILES: c1ccc(-n2c3ccccc3c3cc4c(cc32)c2ccccc2n4-c2ccc([Si](c3ccccc3)(c3ccccc3)c3cccc(-n4c5ccccc5c5ccccc54)c3)cc2)cc1 ; CC1(C)c2ccccc2-c2cc3c(cc21)c1ccccc1n3-c1ccc([Si](c2ccccc2)(c2ccccc2)c2cccc(-n3c4ccccc4c4ccccc43)c2)cc1 ; c1ccc([Si](c2ccccc2)(c2ccc(-n3c4ccccc4c4cc5c(cc43)oc3ccccc35)cc2)c2cccc(-n3c4ccccc4c4ccccc43)c2)cc1 ; c1ccc([Si](c2ccccc2)(c2ccc(-n3c4ccccc4c4cc5c(cc43)sc3ccccc35)cc2)c2cccc(-n3c4ccccc4c4ccccc43)c2)cc1]

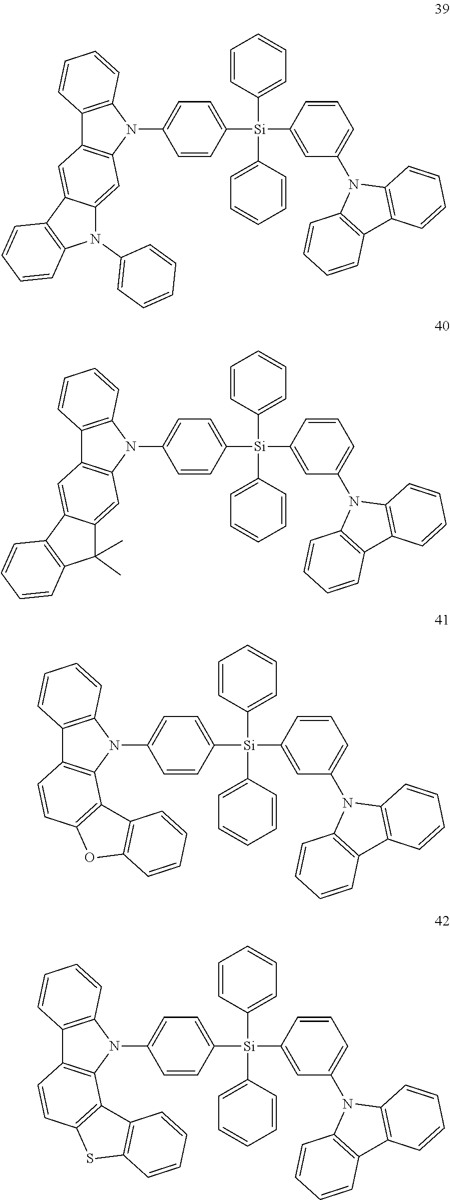 [SMILES: c1ccc(-n2c3ccccc3c3cc4c5ccccc5n(-c5ccc([Si](c6ccccc6)(c6ccccc6)c6cccc(-n7c8ccccc8c8ccccc87)c6)cc5)c4cc32)cc1 ; CC1(C)c2ccccc2-c2cc3c4ccccc4n(-c4ccc([Si](c5ccccc5)(c5ccccc5)c5cccc(-n6c7ccccc7c7ccccc76)c5)cc4)c3cc21 ; c1ccc([Si](c2ccccc2)(c2ccc(-n3c4ccccc4c4ccc5oc6ccccc6c5c43)cc2)c2cccc(-n3c4ccccc4c4ccccc43)c2)cc1 ; c1ccc([Si](c2ccccc2)(c2ccc(-n3c4ccccc4c4ccc5sc6ccccc6c5c43)cc2)c2cccc(-n3c4ccccc4c4ccccc43)c2)cc1]

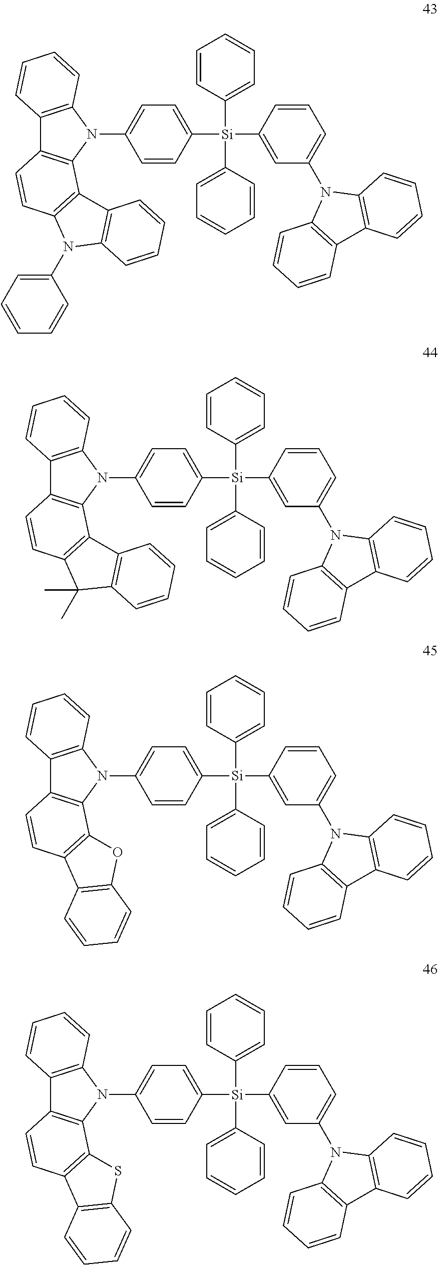 [SMILES: c1ccc(-n2c3ccccc3c3c2ccc2c4ccccc4n(-c4ccc([Si](c5ccccc5)(c5ccccc5)c5cccc(-n6c7ccccc7c7ccccc76)c5)cc4)c23)cc1 ; CC1(C)c2ccccc2-c2c1ccc1c3ccccc3n(-c3ccc([Si](c4ccccc4)(c4ccccc4)c4cccc(-n5c6ccccc6c6ccccc65)c4)cc3)c21 ; c1ccc([Si](c2ccccc2)(c2ccc(-n3c4ccccc4c4ccc5c6ccccc6oc5c43)cc2)c2cccc(-n3c4ccccc4c4ccccc43)c2)cc1 ; c1ccc([Si](c2ccccc2)(c2ccc(-n3c4ccccc4c4ccc5c6ccccc6sc5c43)cc2)c2cccc(-n3c4ccccc4c4ccccc43)c2)cc1]

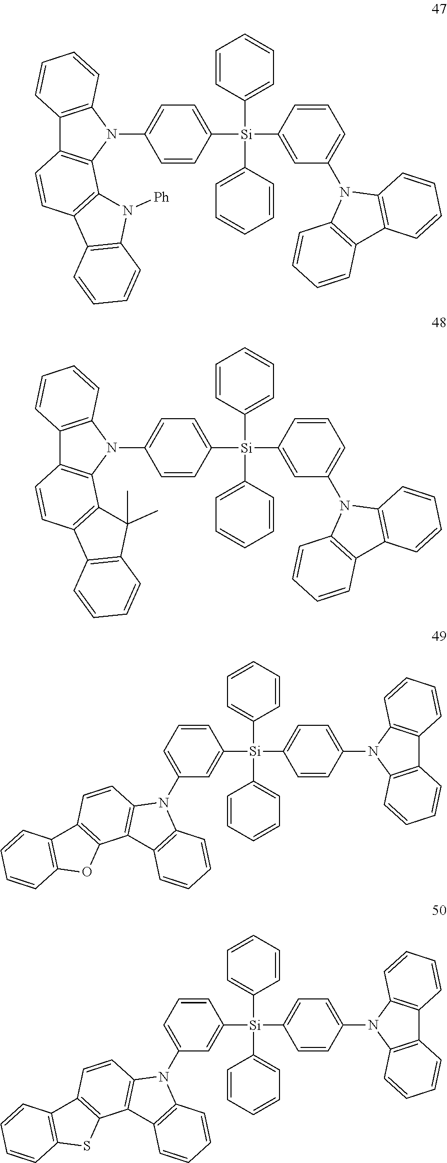 [SMILES: c1ccc(-n2c3ccccc3c3ccc4c5ccccc5n(-c5ccc([Si](c6ccccc6)(c6ccccc6)c6cccc(-n7c8ccccc8c8ccccc87)c6)cc5)c4c32)cc1 ; CC1(C)c2ccccc2-c2ccc3c4ccccc4n(-c4ccc([Si](c5ccccc5)(c5ccccc5)c5cccc(-n6c7ccccc7c7ccccc76)c5)cc4)c3c21 ; c1ccc([Si](c2ccccc2)(c2ccc(-n3c4ccccc4c4ccccc43)cc2)c2cccc(-n3c4ccccc4c4c5oc6ccccc6c5ccc43)c2)cc1 ; c1ccc([Si](c2ccccc2)(c2ccc(-n3c4ccccc4c4ccccc43)cc2)c2cccc(-n3c4ccccc4c4c5sc6ccccc6c5ccc43)c2)cc1]

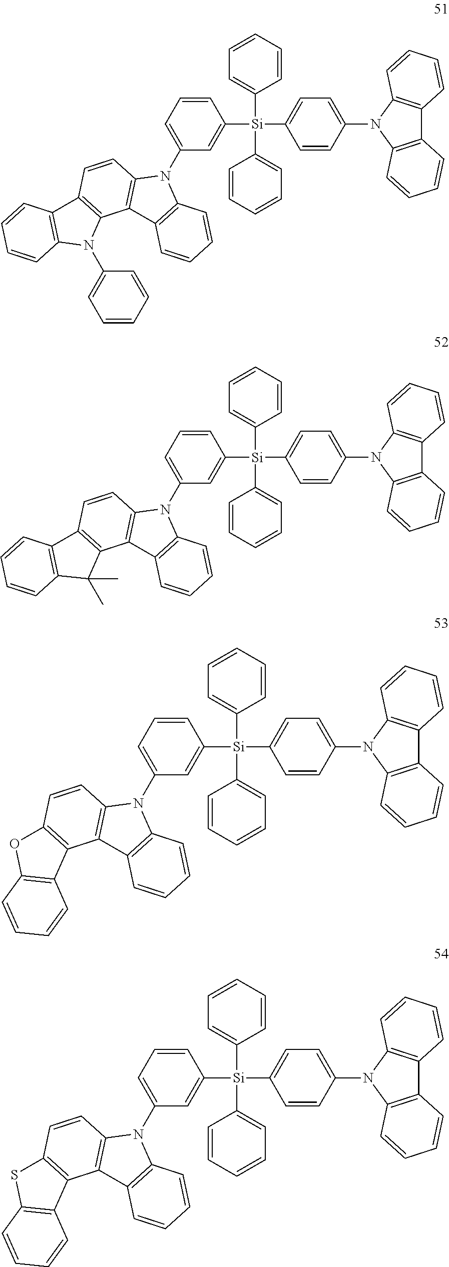 [SMILES: c1ccc(-n2c3ccccc3c3ccc4c(c5ccccc5n4-c4cccc([Si](c5ccccc5)(c5ccccc5)c5ccc(-n6c7ccccc7c7ccccc76)cc5)c4)c32)cc1 ; CC1(C)c2ccccc2-c2ccc3c(c21)c1ccccc1n3-c1cccc([Si](c2ccccc2)(c2ccccc2)c2ccc(-n3c4ccccc4c4ccccc43)cc2)c1 ; c1ccc([Si](c2ccccc2)(c2ccc(-n3c4ccccc4c4ccccc43)cc2)c2cccc(-n3c4ccccc4c4c5c(ccc43)oc3ccccc35)c2)cc1 ; c1ccc([Si](c2ccccc2)(c2ccc(-n3c4ccccc4c4ccccc43)cc2)c2cccc(-n3c4ccccc4c4c5c(ccc43)sc3ccccc35)c2)cc1]

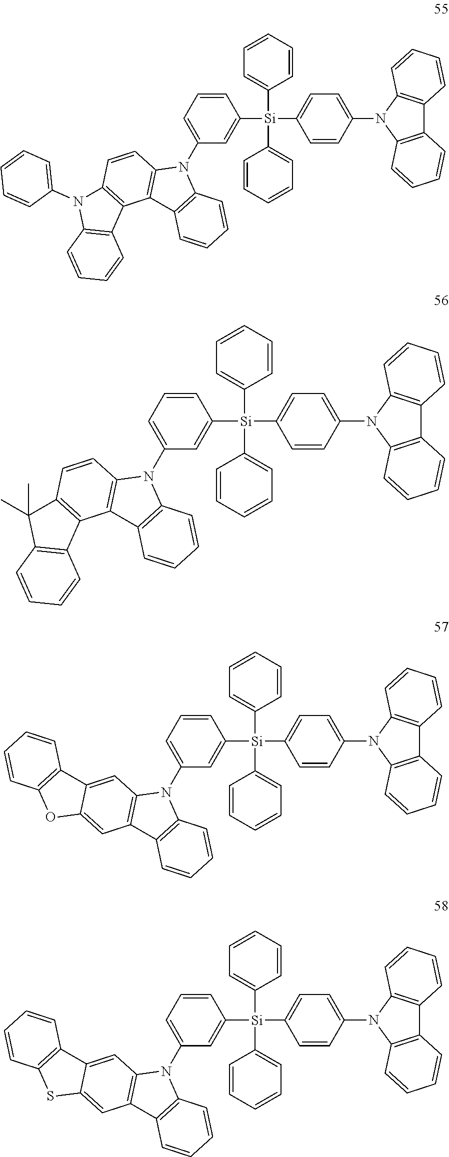 [SMILES: c1ccc(-n2c3ccccc3c3c4c5ccccc5n(-c5cccc([Si](c6ccccc6)(c6ccccc6)c6ccc(-n7c8ccccc8c8ccccc87)cc6)c5)c4ccc32)cc1 ; CC1(C)c2ccccc2-c2c1ccc1c2c2ccccc2n1-c1cccc([Si](c2ccccc2)(c2ccccc2)c2ccc(-n3c4ccccc4c4ccccc43)cc2)c1 ; c1ccc([Si](c2ccccc2)(c2ccc(-n3c4ccccc4c4ccccc43)cc2)c2cccc(-n3c4ccccc4c4cc5oc6ccccc6c5cc43)c2)cc1 ; c1ccc([Si](c2ccccc2)(c2ccc(-n3c4ccccc4c4ccccc43)cc2)c2cccc(-n3c4ccccc4c4cc5sc6ccccc6c5cc43)c2)cc1]

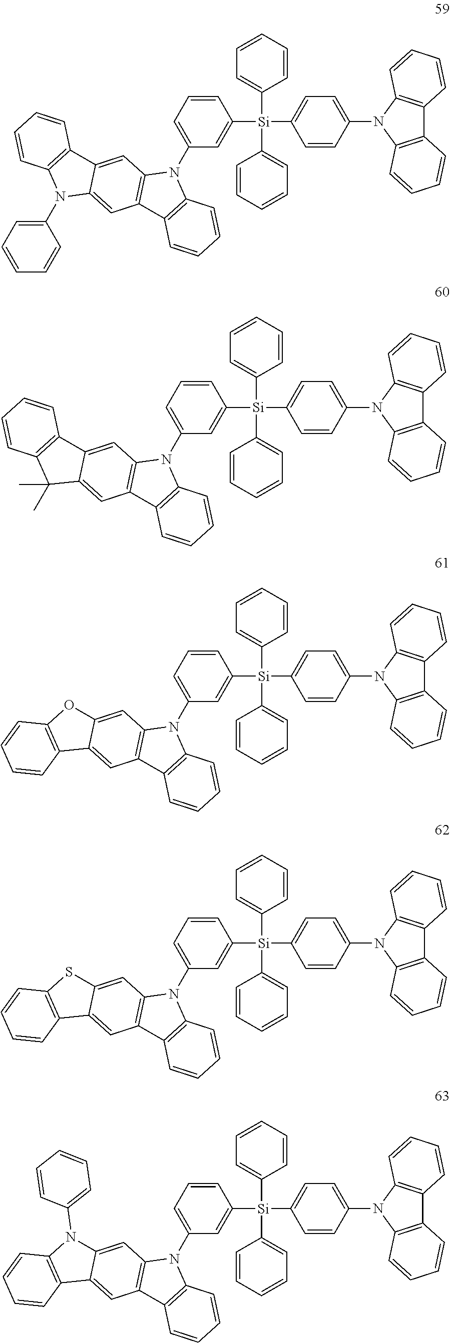

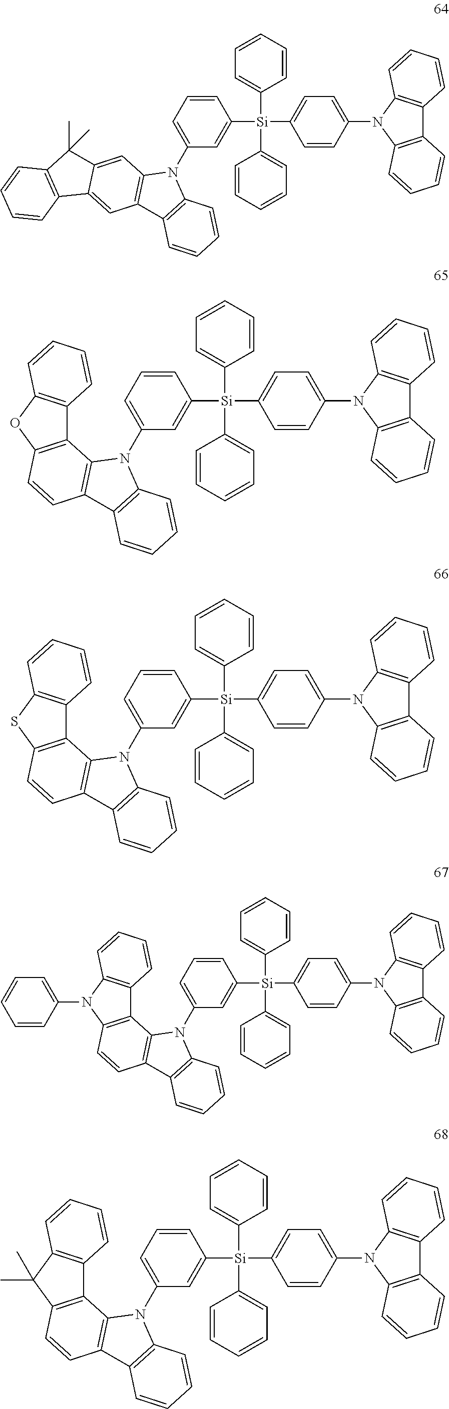

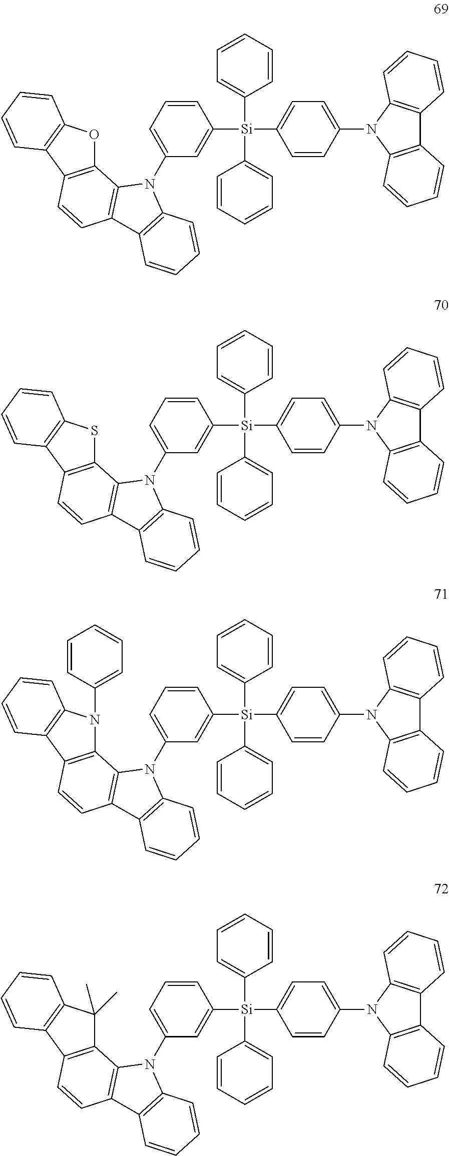 [SMILES: c1ccc([Si](c2ccccc2)(c2ccc(-n3c4ccccc4c4ccccc43)cc2)c2cccc(-n3c4ccccc4c4ccc5c6ccccc6oc5c43)c2)cc1 ; c1ccc([Si](c2ccccc2)(c2ccc(-n3c4ccccc4c4ccccc43)cc2)c2cccc(-n3c4ccccc4c4ccc5c6ccccc6sc5c43)c2)cc1 ; c1ccc(-n2c3ccccc3c3ccc4c5ccccc5n(-c5cccc([Si](c6ccccc6)(c6ccccc6)c6ccc(-n7c8ccccc8c8ccccc87)cc6)c5)c4c32)cc1 ; CC1(C)c2ccccc2-c2ccc3c4ccccc4n(-c4cccc([Si](c5ccccc5)(c5ccccc5)c5ccc(-n6c7ccccc7c7ccccc76)cc5)c4)c3c21]

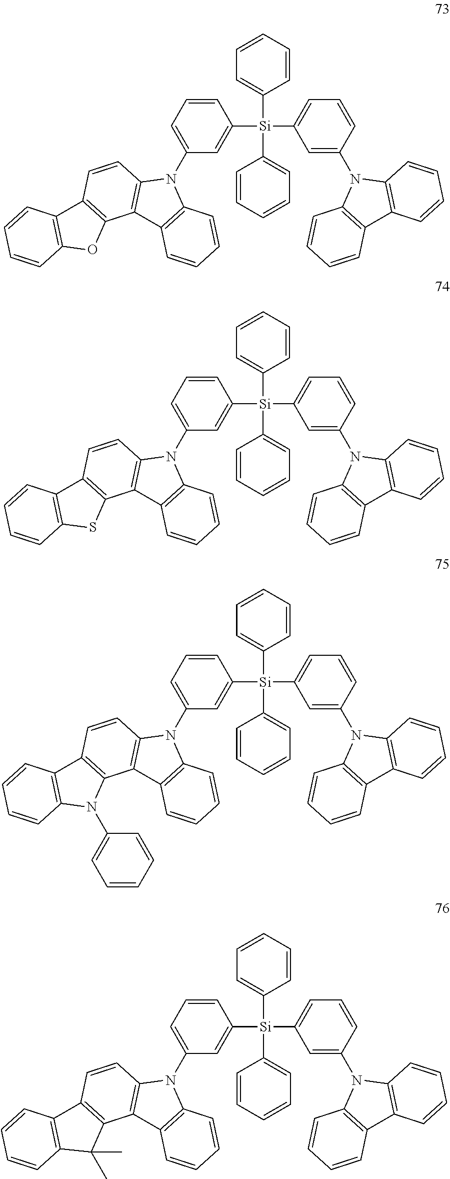 [SMILES: c1ccc([Si](c2ccccc2)(c2cccc(-n3c4ccccc4c4ccccc43)c2)c2cccc(-n3c4ccccc4c4c5oc6ccccc6c5ccc43)c2)cc1 ; c1ccc([Si](c2ccccc2)(c2cccc(-n3c4ccccc4c4ccccc43)c2)c2cccc(-n3c4ccccc4c4c5sc6ccccc6c5ccc43)c2)cc1 ; c1ccc(-n2c3ccccc3c3ccc4c(c5ccccc5n4-c4cccc([Si](c5ccccc5)(c5ccccc5)c5cccc(-n6c7ccccc7c7ccccc76)c5)c4)c32)cc1 ; CC1(C)c2ccccc2-c2ccc3c(c21)c1ccccc1n3-c1cccc([Si](c2ccccc2)(c2ccccc2)c2cccc(-n3c4ccccc4c4ccccc43)c2)c1]

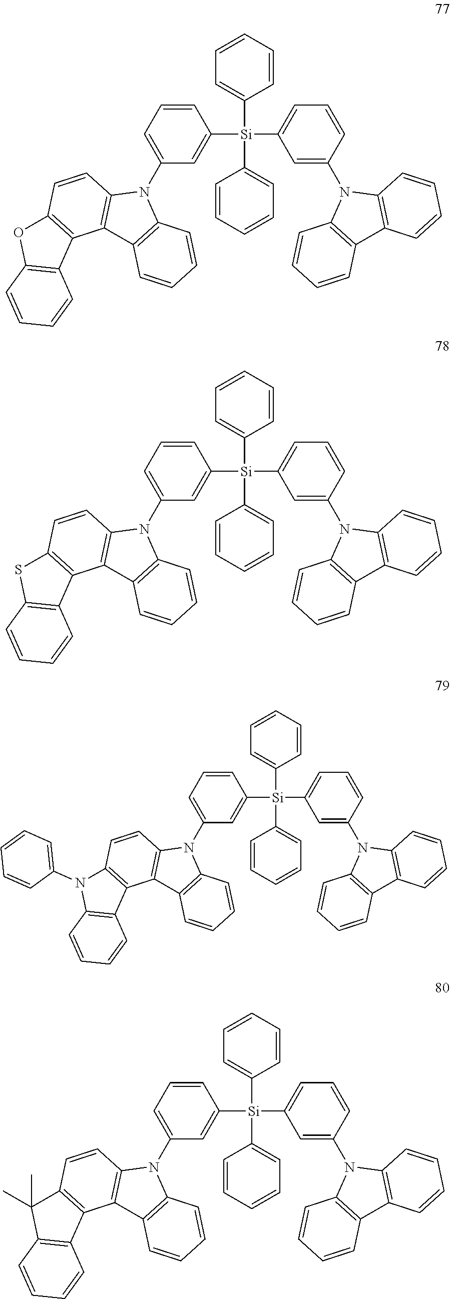 [SMILES: c1ccc([Si](c2ccccc2)(c2cccc(-n3c4ccccc4c4ccccc43)c2)c2cccc(-n3c4ccccc4c4c5c(ccc43)oc3ccccc35)c2)cc1 ; c1ccc([Si](c2ccccc2)(c2cccc(-n3c4ccccc4c4ccccc43)c2)c2cccc(-n3c4ccccc4c4c5c(ccc43)sc3ccccc35)c2)cc1 ; c1ccc(-n2c3ccccc3c3c4c5ccccc5n(-c5cccc([Si](c6ccccc6)(c6ccccc6)c6cccc(-n7c8ccccc8c8ccccc87)c6)c5)c4ccc32)cc1 ; CC1(C)c2ccccc2-c2c1ccc1c2c2ccccc2n1-c1cccc([Si](c2ccccc2)(c2ccccc2)c2cccc(-n3c4ccccc4c4ccccc43)c2)c1]

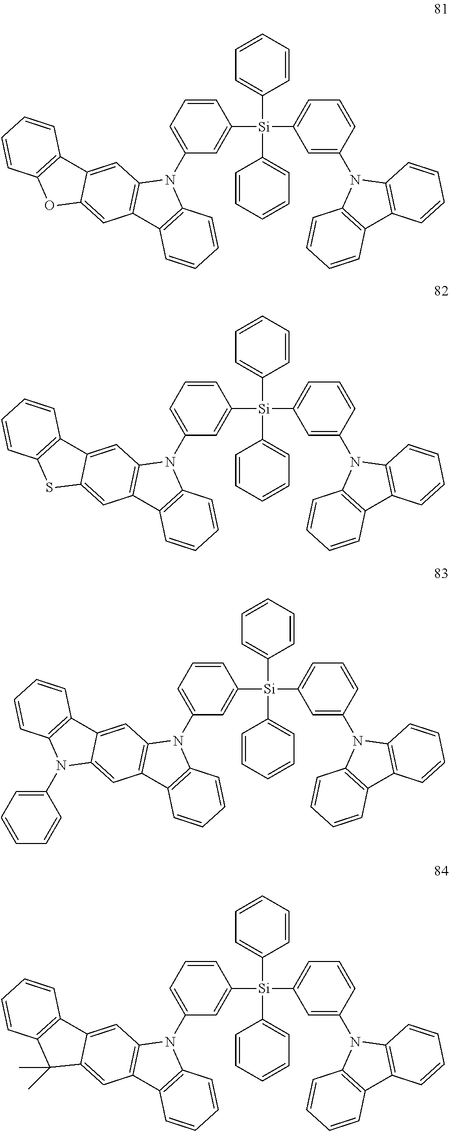 [SMILES: c1ccc([Si](c2ccccc2)(c2cccc(-n3c4ccccc4c4ccccc43)c2)c2cccc(-n3c4ccccc4c4cc5oc6ccccc6c5cc43)c2)cc1 ; c1ccc([Si](c2ccccc2)(c2cccc(-n3c4ccccc4c4ccccc43)c2)c2cccc(-n3c4ccccc4c4cc5sc6ccccc6c5cc43)c2)cc1 ; c1ccc(-n2c3ccccc3c3cc4c(cc32)c2ccccc2n4-c2cccc([Si](c3ccccc3)(c3ccccc3)c3cccc(-n4c5ccccc5c5ccccc54)c3)c2)cc1 ; CC1(C)c2ccccc2-c2cc3c(cc21)c1ccccc1n3-c1cccc([Si](c2ccccc2)(c2ccccc2)c2cccc(-n3c4ccccc4c4ccccc43)c2)c1]

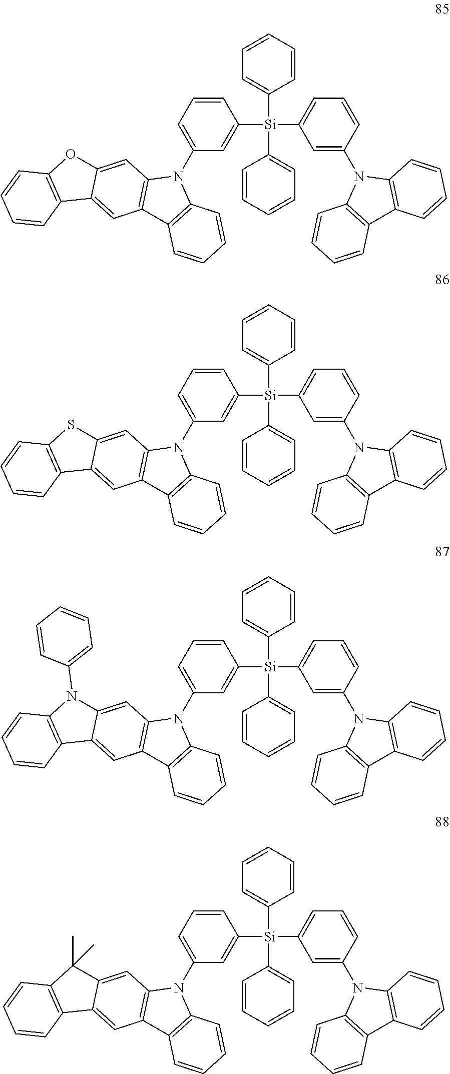 [SMILES: c1ccc([Si](c2ccccc2)(c2cccc(-n3c4ccccc4c4ccccc43)c2)c2cccc(-n3c4ccccc4c4cc5c(cc43)oc3ccccc35)c2)cc1 ; c1ccc([Si](c2ccccc2)(c2cccc(-n3c4ccccc4c4ccccc43)c2)c2cccc(-n3c4ccccc4c4cc5c(cc43)sc3ccccc35)c2)cc1 ; c1ccc(-n2c3ccccc3c3cc4c5ccccc5n(-c5cccc([Si](c6ccccc6)(c6ccccc6)c6cccc(-n7c8ccccc8c8ccccc87)c6)c5)c4cc32)cc1 ; CC1(C)c2ccccc2-c2cc3c4ccccc4n(-c4cccc([Si](c5ccccc5)(c5ccccc5)c5cccc(-n6c7ccccc7c7ccccc76)c5)c4)c3cc21]

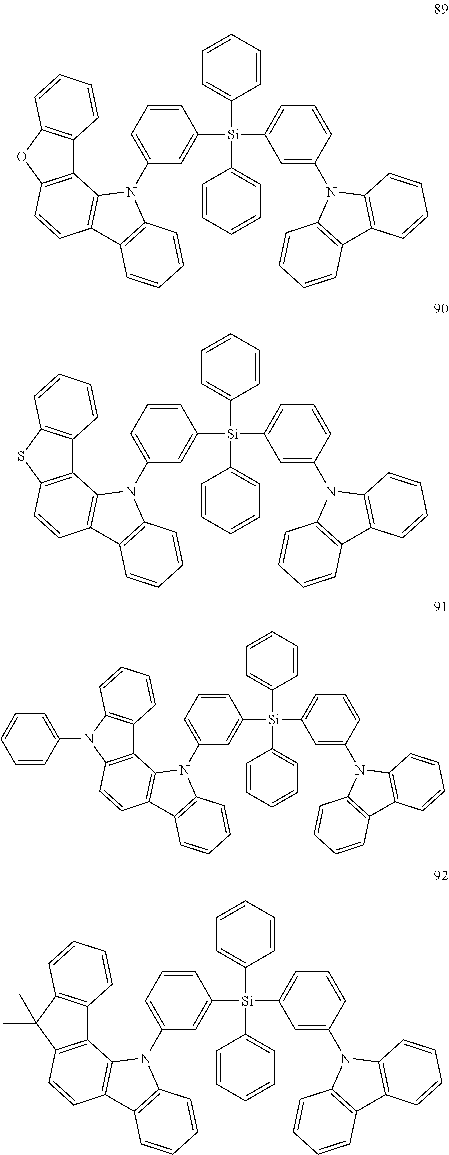 [SMILES: c1ccc([Si](c2ccccc2)(c2cccc(-n3c4ccccc4c4ccccc43)c2)c2cccc(-n3c4ccccc4c4ccc5oc6ccccc6c5c43)c2)cc1 ; c1ccc([Si](c2ccccc2)(c2cccc(-n3c4ccccc4c4ccccc43)c2)c2cccc(-n3c4ccccc4c4ccc5sc6ccccc6c5c43)c2)cc1 ; c1ccc(-n2c3ccccc3c3c2ccc2c4ccccc4n(-c4cccc([Si](c5ccccc5)(c5ccccc5)c5cccc(-n6c7ccccc7c7ccccc76)c5)c4)c23)cc1 ; CC1(C)c2ccccc2-c2c1ccc1c3ccccc3n(-c3cccc([Si](c4ccccc4)(c4ccccc4)c4cccc(-n5c6ccccc6c6ccccc65)c4)c3)c21]

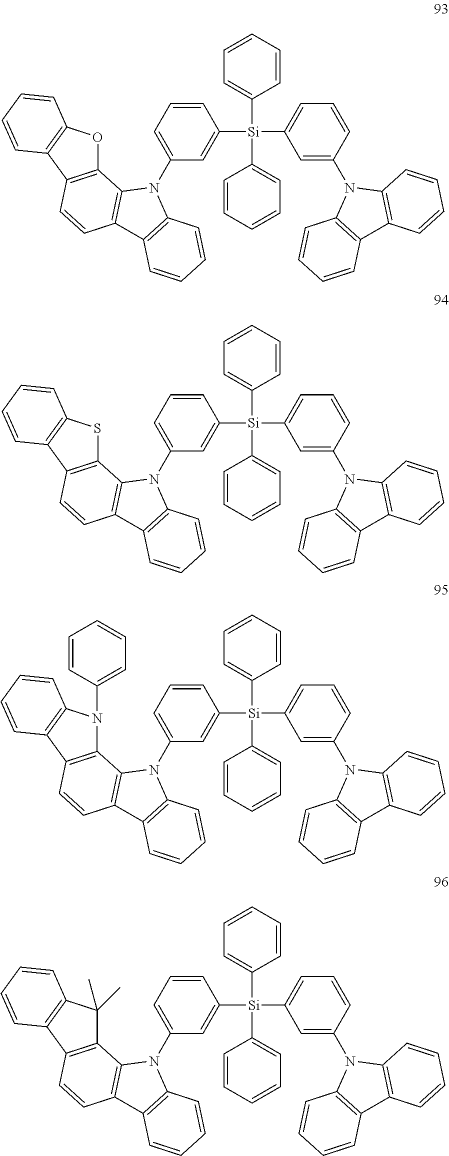 [SMILES: c1ccc([Si](c2ccccc2)(c2cccc(-n3c4ccccc4c4ccccc43)c2)c2cccc(-n3c4ccccc4c4ccc5c6ccccc6oc5c43)c2)cc1 ; c1ccc([Si](c2ccccc2)(c2cccc(-n3c4ccccc4c4ccccc43)c2)c2cccc(-n3c4ccccc4c4ccc5c6ccccc6sc5c43)c2)cc1 ; c1ccc(-n2c3ccccc3c3ccc4c5ccccc5n(-c5cccc([Si](c6ccccc6)(c6ccccc6)c6cccc(-n7c8ccccc8c8ccccc87)c6)c5)c4c32)cc1 ; CC1(C)c2ccccc2-c2ccc3c4ccccc4n(-c4cccc([Si](c5ccccc5)(c5ccccc5)c5cccc(-n6c7ccccc7c7ccccc76)c5)c4)c3c21]

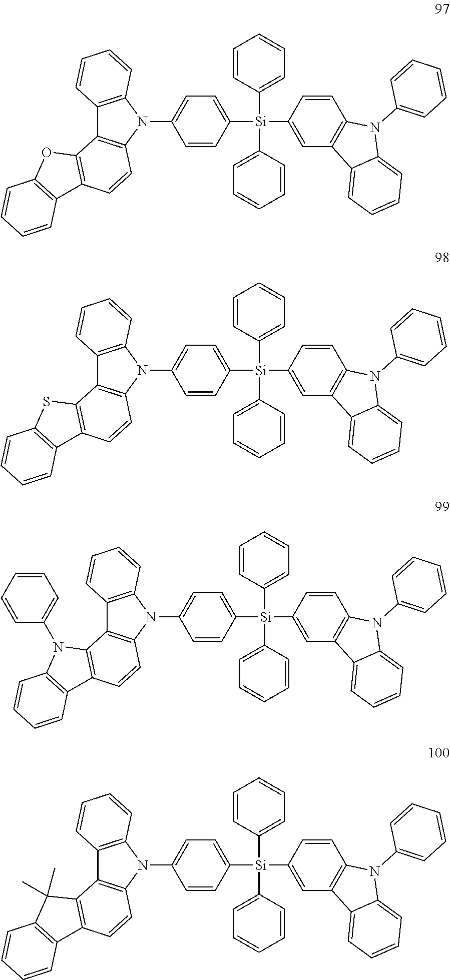 [SMILES: c1ccc(-n2c3ccccc3c3cc([Si](c4ccccc4)(c4ccccc4)c4ccc(-n5c6ccccc6c6c7oc8ccccc8c7ccc65)cc4)ccc32)cc1 ; c1ccc(-n2c3ccccc3c3cc([Si](c4ccccc4)(c4ccccc4)c4ccc(-n5c6ccccc6c6c7sc8ccccc8c7ccc65)cc4)ccc32)cc1 ; c1ccc(-n2c3ccccc3c3cc([Si](c4ccccc4)(c4ccccc4)c4ccc(-n5c6ccccc6c6c5ccc5c7ccccc7n(-c7ccccc7)c56)cc4)ccc32)cc1 ; CC1(C)c2ccccc2-c2ccc3c(c21)c1ccccc1n3-c1ccc([Si](c2ccccc2)(c2ccccc2)c2ccc3c(c2)c2ccccc2n3-c2ccccc2)cc1]

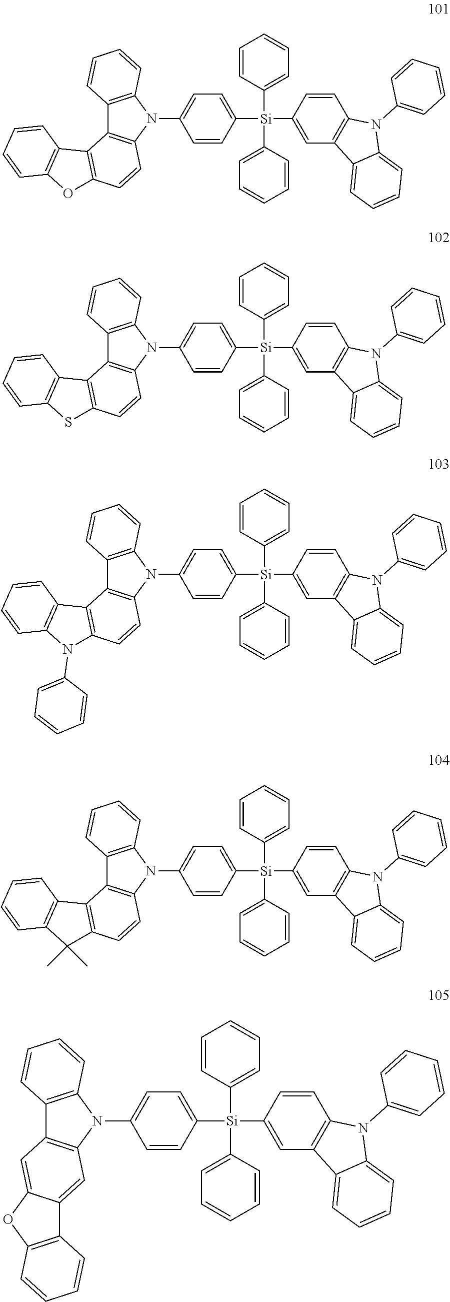

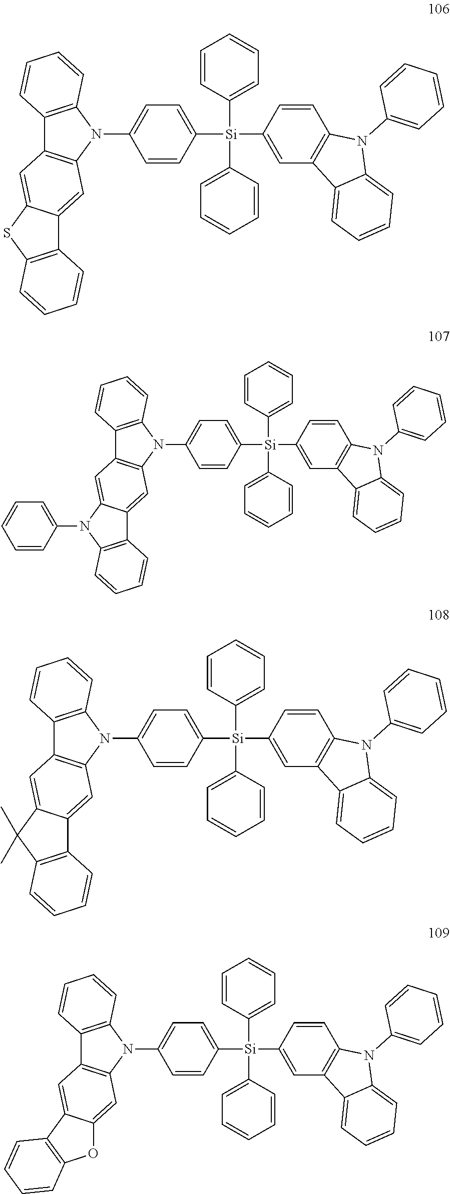 [SMILES: c1ccc(-n2c3ccccc3c3cc([Si](c4ccccc4)(c4ccccc4)c4ccc(-n5c6ccccc6c6cc7sc8ccccc8c7cc65)cc4)ccc32)cc1 ; c1ccc(-n2c3ccccc3c3cc([Si](c4ccccc4)(c4ccccc4)c4ccc(-n5c6ccccc6c6cc7c(cc65)c5ccccc5n7-c5ccccc5)cc4)ccc32)cc1 ; CC1(C)c2ccccc2-c2cc3c(cc21)c1ccccc1n3-c1ccc([Si](c2ccccc2)(c2ccccc2)c2ccc3c(c2)c2ccccc2n3-c2ccccc2)cc1 ; c1ccc(-n2c3ccccc3c3cc([Si](c4ccccc4)(c4ccccc4)c4ccc(-n5c6ccccc6c6cc7c(cc65)oc5ccccc57)cc4)ccc32)cc1]

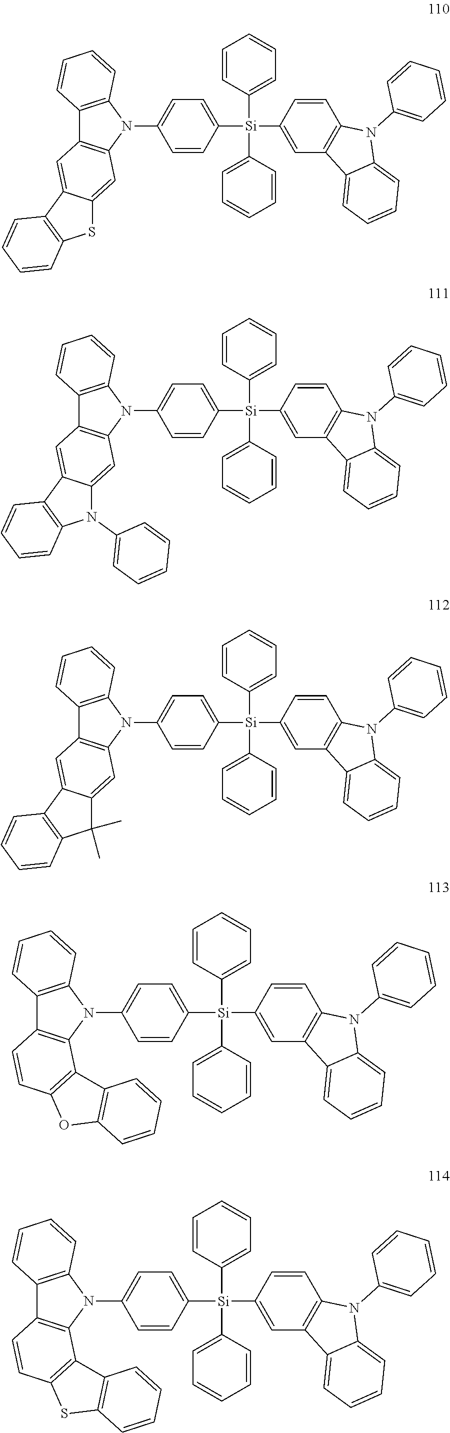

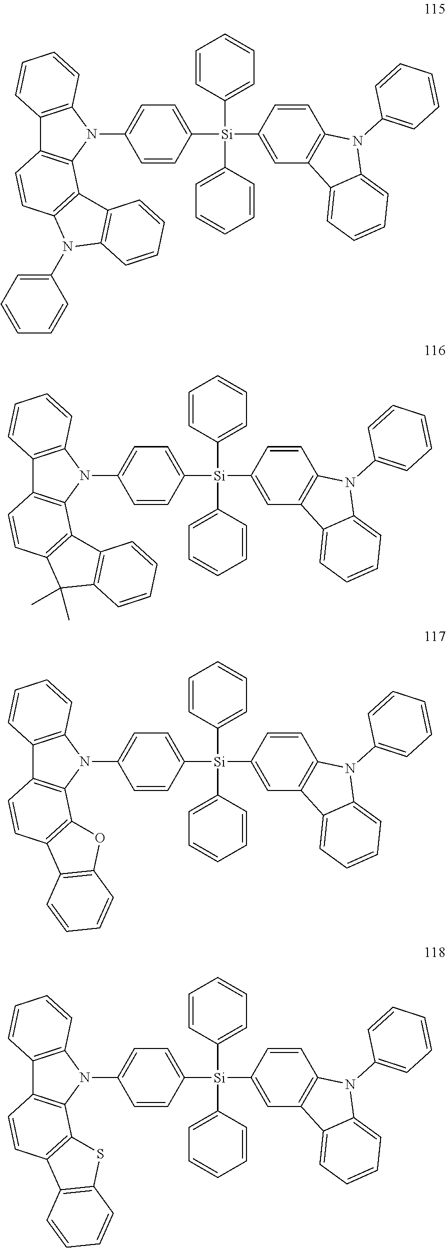 [SMILES: c1ccc(-n2c3ccccc3c3cc([Si](c4ccccc4)(c4ccccc4)c4ccc(-n5c6ccccc6c6ccc7c(c8ccccc8n7-c7ccccc7)c65)cc4)ccc32)cc1 ; CC1(C)c2ccccc2-c2c1ccc1c3ccccc3n(-c3ccc([Si](c4ccccc4)(c4ccccc4)c4ccc5c(c4)c4ccccc4n5-c4ccccc4)cc3)c21 ; c1ccc(-n2c3ccccc3c3cc([Si](c4ccccc4)(c4ccccc4)c4ccc(-n5c6ccccc6c6ccc7c8ccccc8oc7c65)cc4)ccc32)cc1 ; c1ccc(-n2c3ccccc3c3cc([Si](c4ccccc4)(c4ccccc4)c4ccc(-n5c6ccccc6c6ccc7c8ccccc8sc7c65)cc4)ccc32)cc1]

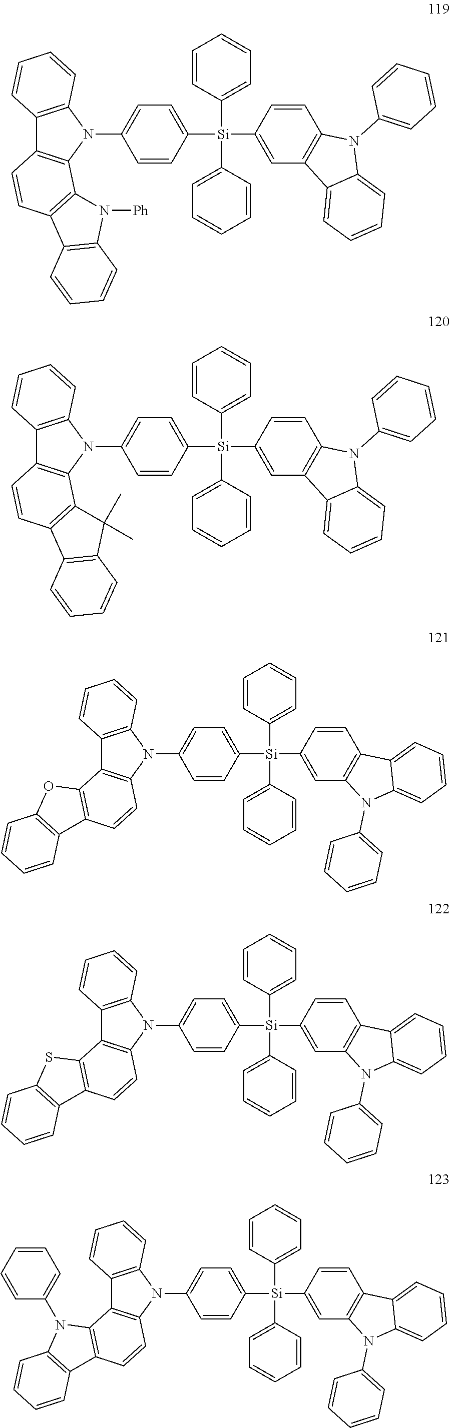

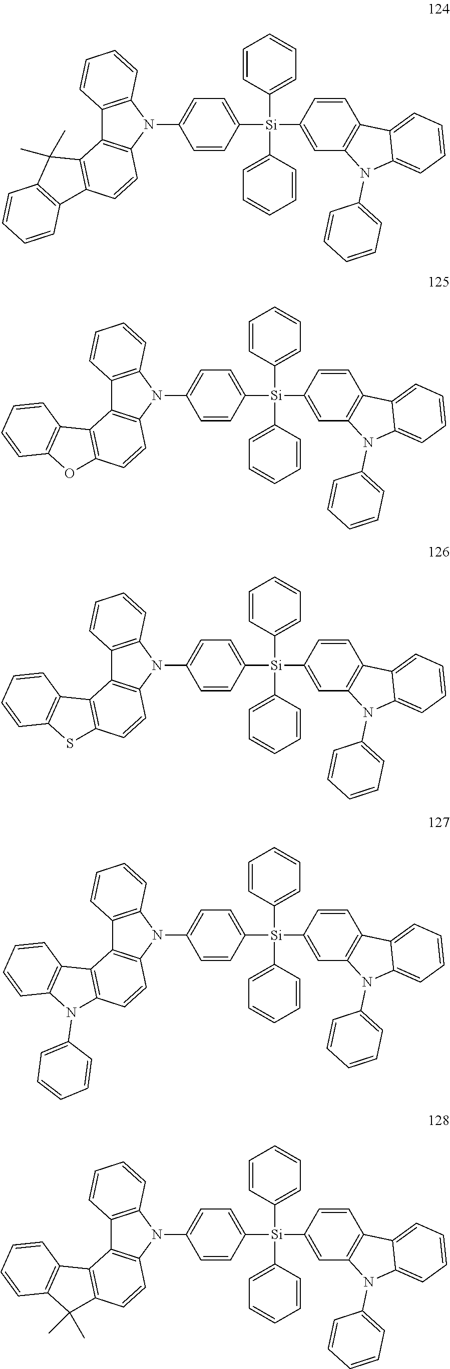

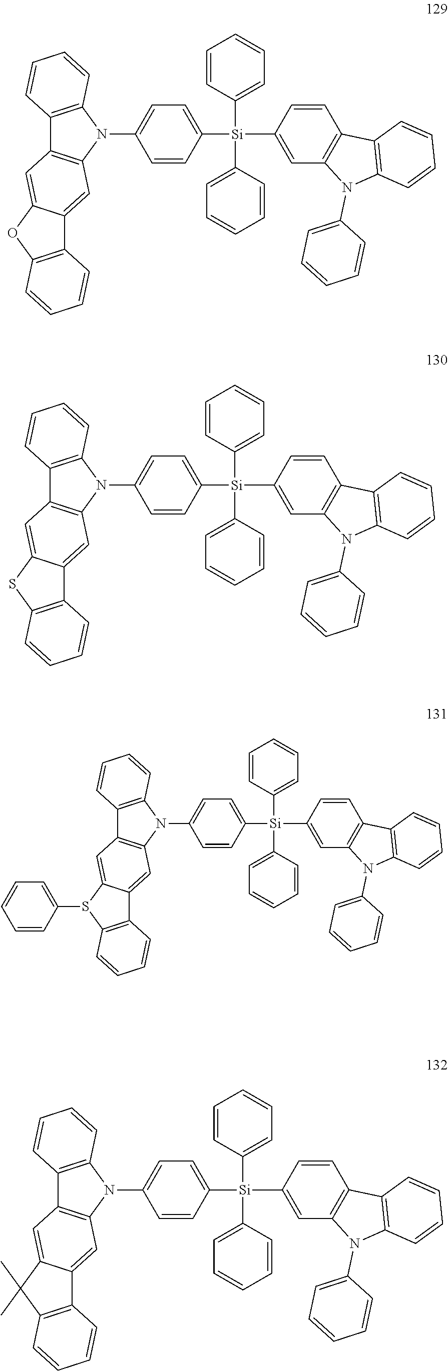

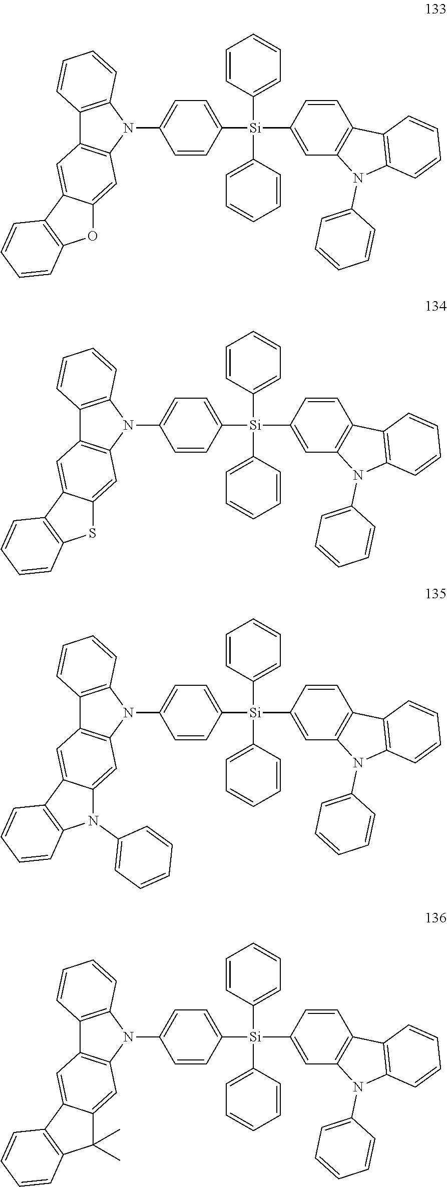 [SMILES: c1ccc(-n2c3ccccc3c3ccc([Si](c4ccccc4)(c4ccccc4)c4ccc(-n5c6ccccc6c6cc7c(cc65)oc5ccccc57)cc4)cc32)cc1 ; c1ccc(-n2c3ccccc3c3ccc([Si](c4ccccc4)(c4ccccc4)c4ccc(-n5c6ccccc6c6cc7c(cc65)sc5ccccc57)cc4)cc32)cc1 ; c1ccc(-n2c3ccccc3c3ccc([Si](c4ccccc4)(c4ccccc4)c4ccc(-n5c6ccccc6c6cc7c8ccccc8n(-c8ccccc8)c7cc65)cc4)cc32)cc1 ; CC1(C)c2ccccc2-c2cc3c4ccccc4n(-c4ccc([Si](c5ccccc5)(c5ccccc5)c5ccc6c7ccccc7n(-c7ccccc7)c6c5)cc4)c3cc21]

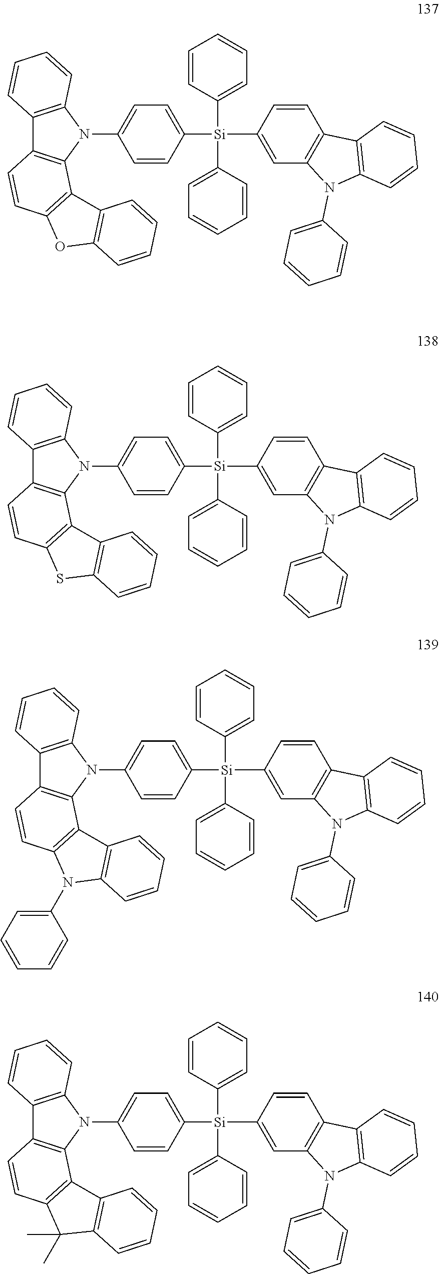 [SMILES: c1ccc(-n2c3ccccc3c3ccc([Si](c4ccccc4)(c4ccccc4)c4ccc(-n5c6ccccc6c6ccc7oc8ccccc8c7c65)cc4)cc32)cc1 ; c1ccc(-n2c3ccccc3c3ccc([Si](c4ccccc4)(c4ccccc4)c4ccc(-n5c6ccccc6c6ccc7sc8ccccc8c7c65)cc4)cc32)cc1 ; c1ccc(-n2c3ccccc3c3ccc([Si](c4ccccc4)(c4ccccc4)c4ccc(-n5c6ccccc6c6ccc7c(c8ccccc8n7-c7ccccc7)c65)cc4)cc32)cc1 ; CC1(C)c2ccccc2-c2c1ccc1c3ccccc3n(-c3ccc([Si](c4ccccc4)(c4ccccc4)c4ccc5c6ccccc6n(-c6ccccc6)c5c4)cc3)c21]

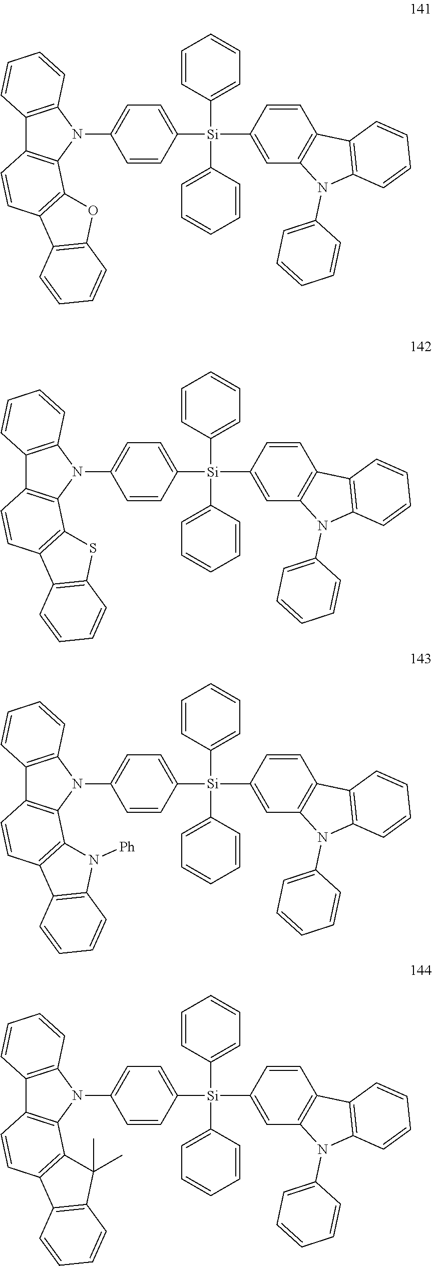 [SMILES: c1ccc(-n2c3ccccc3c3ccc([Si](c4ccccc4)(c4ccccc4)c4ccc(-n5c6ccccc6c6ccc7c8ccccc8oc7c65)cc4)cc32)cc1 ; c1ccc(-n2c3ccccc3c3ccc([Si](c4ccccc4)(c4ccccc4)c4ccc(-n5c6ccccc6c6ccc7c8ccccc8sc7c65)cc4)cc32)cc1 ; c1ccc(-n2c3ccccc3c3ccc([Si](c4ccccc4)(c4ccccc4)c4ccc(-n5c6ccccc6c6ccc7c8ccccc8n(-c8ccccc8)c7c65)cc4)cc32)cc1 ; CC1(C)c2ccccc2-c2ccc3c4ccccc4n(-c4ccc([Si](c5ccccc5)(c5ccccc5)c5ccc6c7ccccc7n(-c7ccccc7)c6c5)cc4)c3c21]

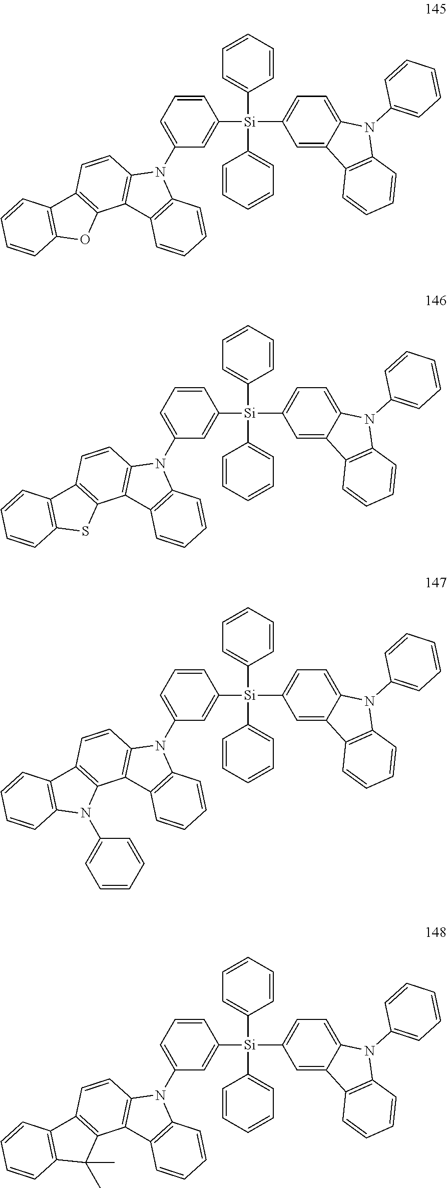 [SMILES: c1ccc(-n2c3ccccc3c3cc([Si](c4ccccc4)(c4ccccc4)c4cccc(-n5c6ccccc6c6c7oc8ccccc8c7ccc65)c4)ccc32)cc1 ; c1ccc(-n2c3ccccc3c3cc([Si](c4ccccc4)(c4ccccc4)c4cccc(-n5c6ccccc6c6c7sc8ccccc8c7ccc65)c4)ccc32)cc1 ; c1ccc(-n2c3ccccc3c3cc([Si](c4ccccc4)(c4ccccc4)c4cccc(-n5c6ccccc6c6c5ccc5c7ccccc7n(-c7ccccc7)c56)c4)ccc32)cc1 ; CC1(C)c2ccccc2-c2ccc3c(c21)c1ccccc1n3-c1cccc([Si](c2ccccc2)(c2ccccc2)c2ccc3c(c2)c2ccccc2n3-c2ccccc2)c1]

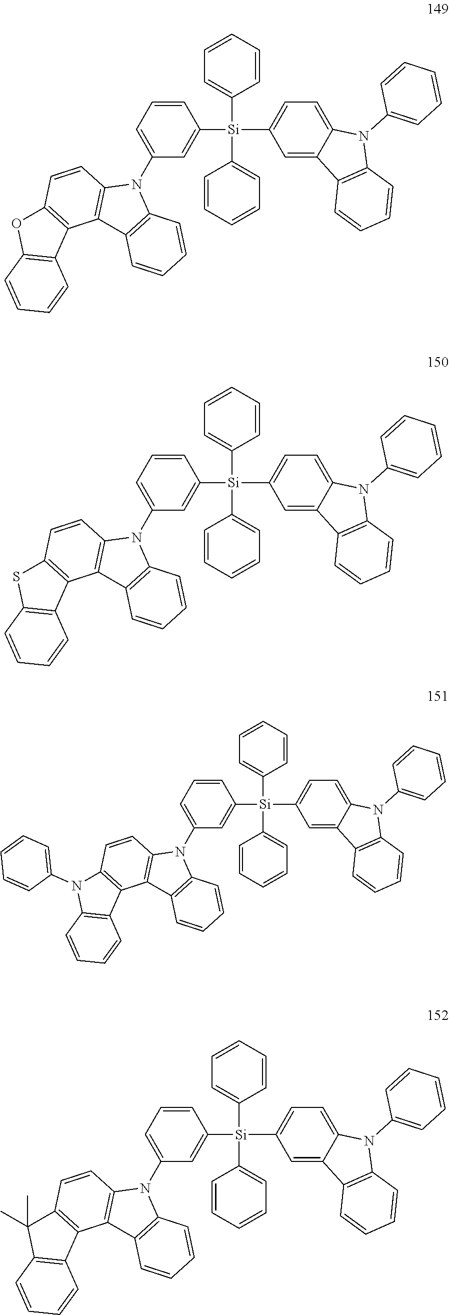 [SMILES: c1ccc(-n2c3ccccc3c3cc([Si](c4ccccc4)(c4ccccc4)c4cccc(-n5c6ccccc6c6c7c(ccc65)oc5ccccc57)c4)ccc32)cc1 ; c1ccc(-n2c3ccccc3c3cc([Si](c4ccccc4)(c4ccccc4)c4cccc(-n5c6ccccc6c6c7c(ccc65)sc5ccccc57)c4)ccc32)cc1 ; c1ccc(-n2c3ccccc3c3cc([Si](c4ccccc4)(c4ccccc4)c4cccc(-n5c6ccccc6c6c7c8ccccc8n(-c8ccccc8)c7ccc65)c4)ccc32)cc1 ; CC1(C)c2ccccc2-c2c1ccc1c2c2ccccc2n1-c1cccc([Si](c2ccccc2)(c2ccccc2)c2ccc3c(c2)c2ccccc2n3-c2ccccc2)c1]

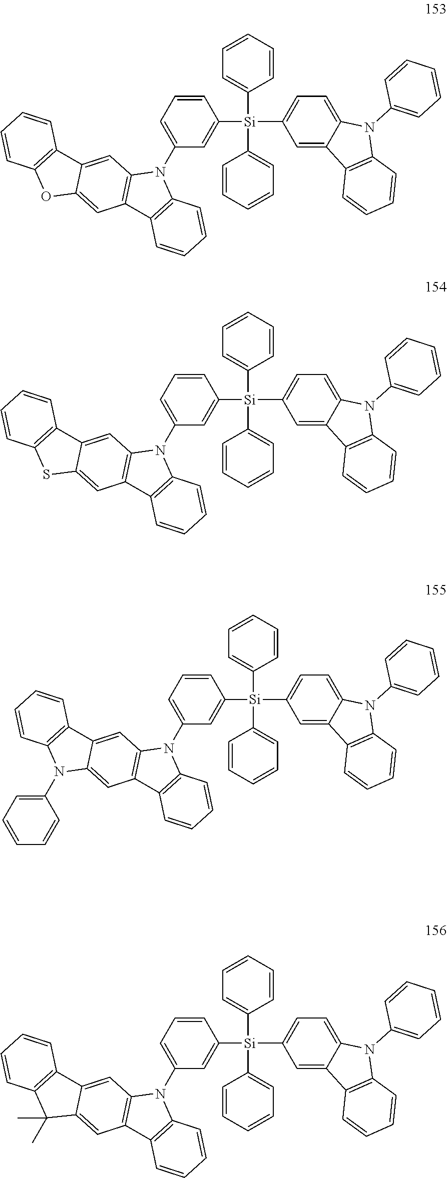 [SMILES: c1ccc(-n2c3ccccc3c3cc([Si](c4ccccc4)(c4ccccc4)c4cccc(-n5c6ccccc6c6cc7oc8ccccc8c7cc65)c4)ccc32)cc1 ; c1ccc(-n2c3ccccc3c3cc([Si](c4ccccc4)(c4ccccc4)c4cccc(-n5c6ccccc6c6cc7sc8ccccc8c7cc65)c4)ccc32)cc1 ; c1ccc(-n2c3ccccc3c3cc([Si](c4ccccc4)(c4ccccc4)c4cccc(-n5c6ccccc6c6cc7c(cc65)c5ccccc5n7-c5ccccc5)c4)ccc32)cc1 ; CC1(C)c2ccccc2-c2cc3c(cc21)c1ccccc1n3-c1cccc([Si](c2ccccc2)(c2ccccc2)c2ccc3c(c2)c2ccccc2n3-c2ccccc2)c1]

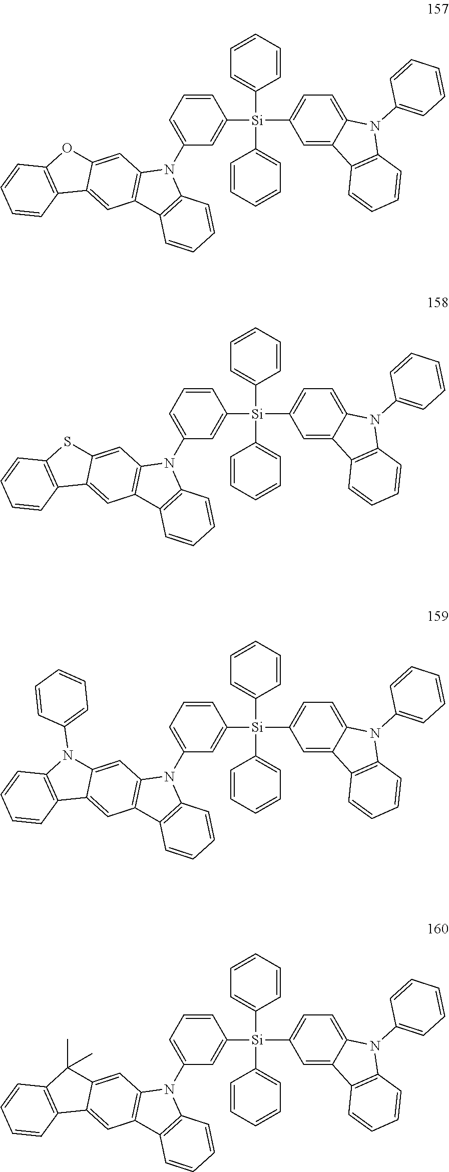 [SMILES: c1ccc(-n2c3ccccc3c3cc([Si](c4ccccc4)(c4ccccc4)c4cccc(-n5c6ccccc6c6cc7c(cc65)oc5ccccc57)c4)ccc32)cc1 ; c1ccc(-n2c3ccccc3c3cc([Si](c4ccccc4)(c4ccccc4)c4cccc(-n5c6ccccc6c6cc7c(cc65)sc5ccccc57)c4)ccc32)cc1 ; c1ccc(-n2c3ccccc3c3cc([Si](c4ccccc4)(c4ccccc4)c4cccc(-n5c6ccccc6c6cc7c8ccccc8n(-c8ccccc8)c7cc65)c4)ccc32)cc1 ; CC1(C)c2ccccc2-c2cc3c4ccccc4n(-c4cccc([Si](c5ccccc5)(c5ccccc5)c5ccc6c(c5)c5ccccc5n6-c5ccccc5)c4)c3cc21]

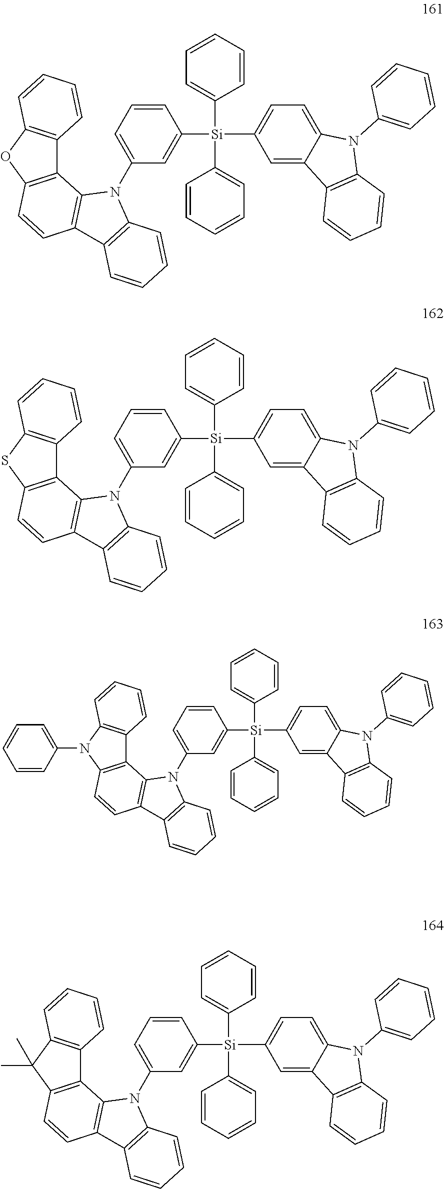 [SMILES: c1ccc(-n2c3ccccc3c3cc([Si](c4ccccc4)(c4ccccc4)c4cccc(-n5c6ccccc6c6ccc7oc8ccccc8c7c65)c4)ccc32)cc1 ; c1ccc(-n2c3ccccc3c3cc([Si](c4ccccc4)(c4ccccc4)c4cccc(-n5c6ccccc6c6ccc7sc8ccccc8c7c65)c4)ccc32)cc1 ; c1ccc(-n2c3ccccc3c3cc([Si](c4ccccc4)(c4ccccc4)c4cccc(-n5c6ccccc6c6ccc7c(c8ccccc8n7-c7ccccc7)c65)c4)ccc32)cc1 ; CC1(C)c2ccccc2-c2c1ccc1c3ccccc3n(-c3cccc([Si](c4ccccc4)(c4ccccc4)c4ccc5c(c4)c4ccccc4n5-c4ccccc4)c3)c21]

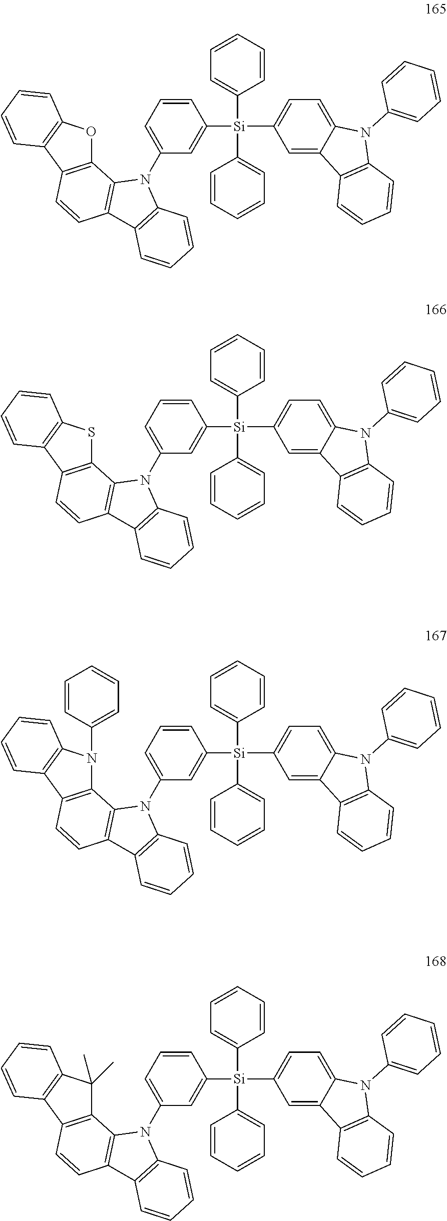 [SMILES: c1ccc(-n2c3ccccc3c3cc([Si](c4ccccc4)(c4ccccc4)c4cccc(-n5c6ccccc6c6ccc7c8ccccc8oc7c65)c4)ccc32)cc1 ; c1ccc(-n2c3ccccc3c3cc([Si](c4ccccc4)(c4ccccc4)c4cccc(-n5c6ccccc6c6ccc7c8ccccc8sc7c65)c4)ccc32)cc1 ; c1ccc(-n2c3ccccc3c3cc([Si](c4ccccc4)(c4ccccc4)c4cccc(-n5c6ccccc6c6ccc7c8ccccc8n(-c8ccccc8)c7c65)c4)ccc32)cc1 ; CC1(C)c2ccccc2-c2ccc3c4ccccc4n(-c4cccc([Si](c5ccccc5)(c5ccccc5)c5ccc6c(c5)c5ccccc5n6-c5ccccc5)c4)c3c21]

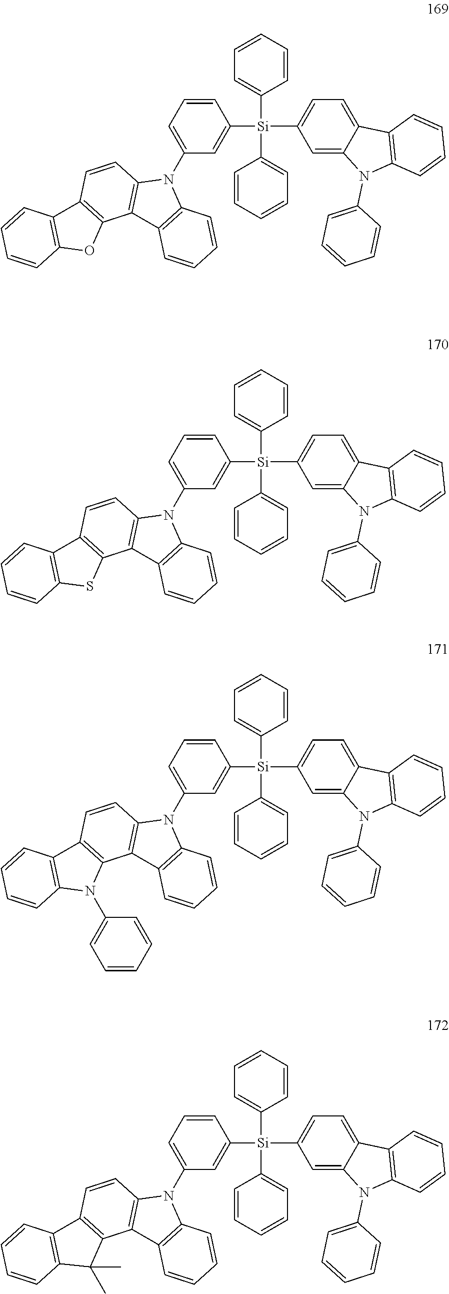 [SMILES: c1ccc(-n2c3ccccc3c3ccc([Si](c4ccccc4)(c4ccccc4)c4cccc(-n5c6ccccc6c6c7oc8ccccc8c7ccc65)c4)cc32)cc1 ; c1ccc(-n2c3ccccc3c3ccc([Si](c4ccccc4)(c4ccccc4)c4cccc(-n5c6ccccc6c6c7sc8ccccc8c7ccc65)c4)cc32)cc1 ; c1ccc(-n2c3ccccc3c3ccc([Si](c4ccccc4)(c4ccccc4)c4cccc(-n5c6ccccc6c6c5ccc5c7ccccc7n(-c7ccccc7)c56)c4)cc32)cc1 ; CC1(C)c2ccccc2-c2ccc3c(c21)c1ccccc1n3-c1cccc([Si](c2ccccc2)(c2ccccc2)c2ccc3c4ccccc4n(-c4ccccc4)c3c2)c1]

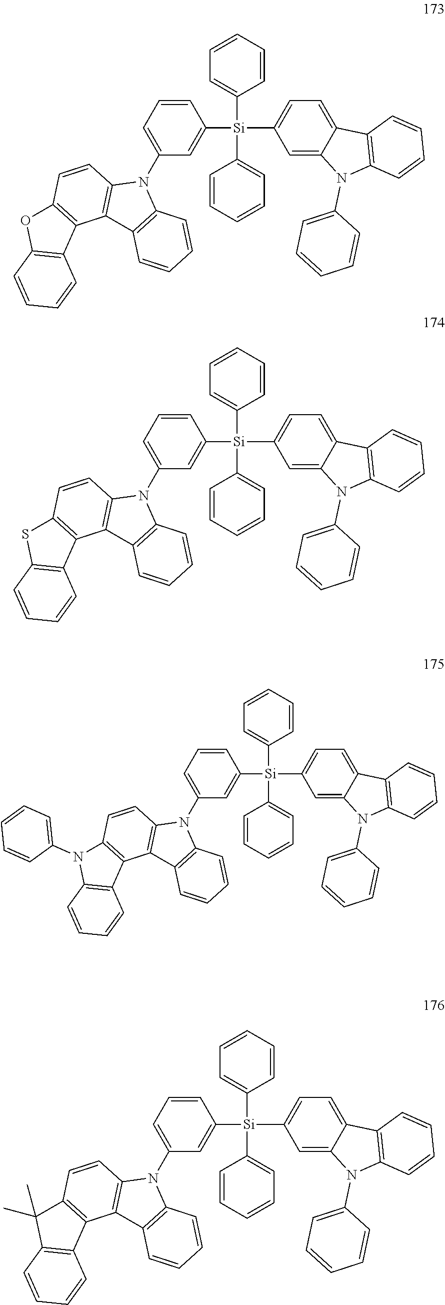 [SMILES: c1ccc(-n2c3ccccc3c3ccc([Si](c4ccccc4)(c4ccccc4)c4cccc(-n5c6ccccc6c6c7c(ccc65)oc5ccccc57)c4)cc32)cc1 ; c1ccc(-n2c3ccccc3c3ccc([Si](c4ccccc4)(c4ccccc4)c4cccc(-n5c6ccccc6c6c7c(ccc65)sc5ccccc57)c4)cc32)cc1 ; c1ccc(-n2c3ccccc3c3ccc([Si](c4ccccc4)(c4ccccc4)c4cccc(-n5c6ccccc6c6c7c8ccccc8n(-c8ccccc8)c7ccc65)c4)cc32)cc1 ; CC1(C)c2ccccc2-c2c1ccc1c2c2ccccc2n1-c1cccc([Si](c2ccccc2)(c2ccccc2)c2ccc3c4ccccc4n(-c4ccccc4)c3c2)c1]

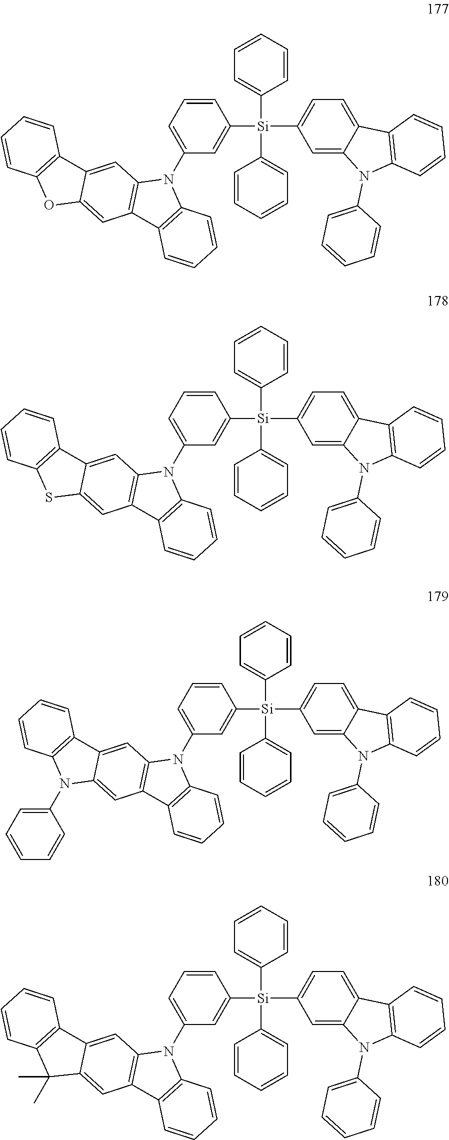 [SMILES: c1ccc(-n2c3ccccc3c3ccc([Si](c4ccccc4)(c4ccccc4)c4cccc(-n5c6ccccc6c6cc7oc8ccccc8c7cc65)c4)cc32)cc1 ; c1ccc(-n2c3ccccc3c3ccc([Si](c4ccccc4)(c4ccccc4)c4cccc(-n5c6ccccc6c6cc7sc8ccccc8c7cc65)c4)cc32)cc1 ; c1ccc(-n2c3ccccc3c3ccc([Si](c4ccccc4)(c4ccccc4)c4cccc(-n5c6ccccc6c6cc7c(cc65)c5ccccc5n7-c5ccccc5)c4)cc32)cc1 ; CC1(C)c2ccccc2-c2cc3c(cc21)c1ccccc1n3-c1cccc([Si](c2ccccc2)(c2ccccc2)c2ccc3c4ccccc4n(-c4ccccc4)c3c2)c1]

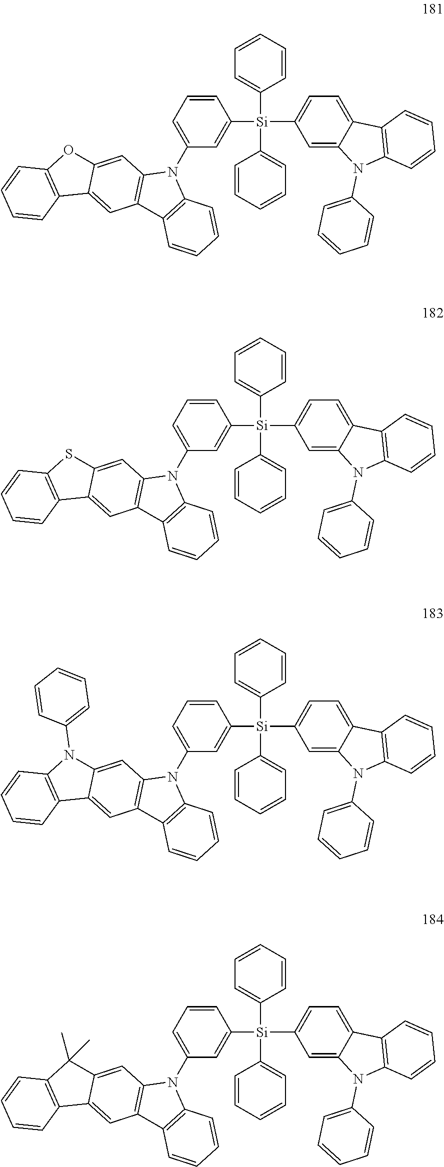 [SMILES: c1ccc(-n2c3ccccc3c3ccc([Si](c4ccccc4)(c4ccccc4)c4cccc(-n5c6ccccc6c6cc7c(cc65)oc5ccccc57)c4)cc32)cc1 ; c1ccc(-n2c3ccccc3c3ccc([Si](c4ccccc4)(c4ccccc4)c4cccc(-n5c6ccccc6c6cc7c(cc65)sc5ccccc57)c4)cc32)cc1 ; c1ccc(-n2c3ccccc3c3ccc([Si](c4ccccc4)(c4ccccc4)c4cccc(-n5c6ccccc6c6cc7c8ccccc8n(-c8ccccc8)c7cc65)c4)cc32)cc1 ; CC1(C)c2ccccc2-c2cc3c4ccccc4n(-c4cccc([Si](c5ccccc5)(c5ccccc5)c5ccc6c7ccccc7n(-c7ccccc7)c6c5)c4)c3cc21]

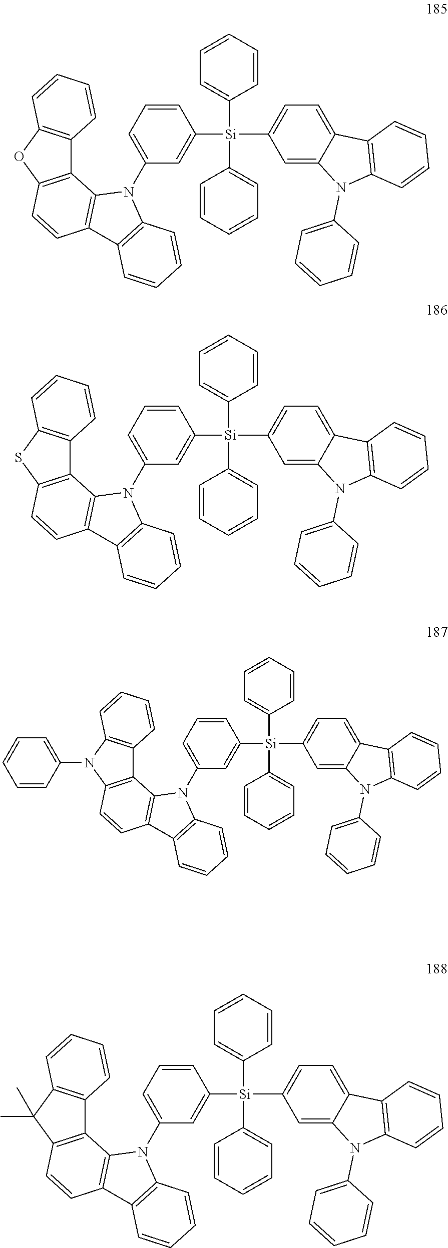 [SMILES: c1ccc(-n2c3ccccc3c3ccc([Si](c4ccccc4)(c4ccccc4)c4cccc(-n5c6ccccc6c6ccc7oc8ccccc8c7c65)c4)cc32)cc1 ; c1ccc(-n2c3ccccc3c3ccc([Si](c4ccccc4)(c4ccccc4)c4cccc(-n5c6ccccc6c6ccc7sc8ccccc8c7c65)c4)cc32)cc1 ; c1ccc(-n2c3ccccc3c3ccc([Si](c4ccccc4)(c4ccccc4)c4cccc(-n5c6ccccc6c6ccc7c(c8ccccc8n7-c7ccccc7)c65)c4)cc32)cc1 ; CC1(C)c2ccccc2-c2c1ccc1c3ccccc3n(-c3cccc([Si](c4ccccc4)(c4ccccc4)c4ccc5c6ccccc6n(-c6ccccc6)c5c4)c3)c21]

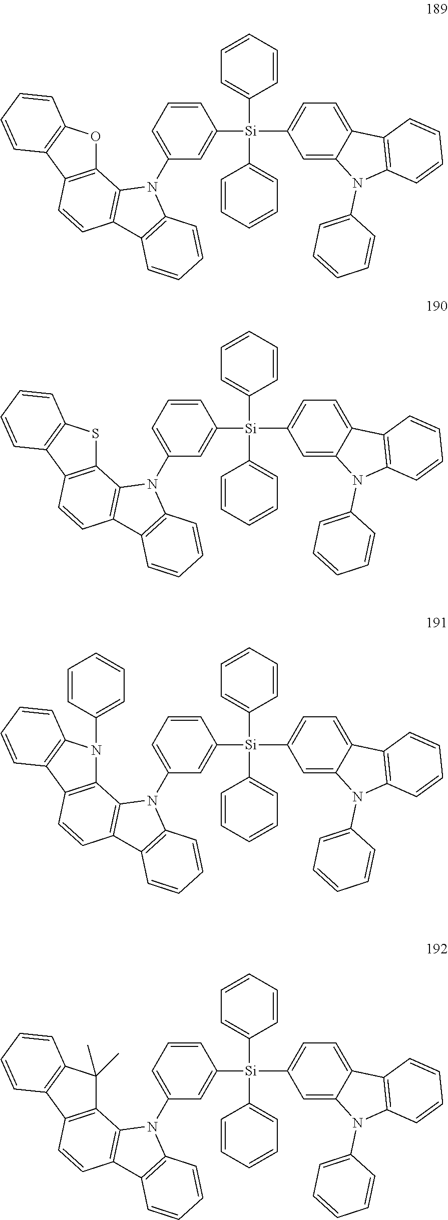 [SMILES: c1ccc(-n2c3ccccc3c3ccc([Si](c4ccccc4)(c4ccccc4)c4cccc(-n5c6ccccc6c6ccc7c8ccccc8oc7c65)c4)cc32)cc1 ; c1ccc(-n2c3ccccc3c3ccc([Si](c4ccccc4)(c4ccccc4)c4cccc(-n5c6ccccc6c6ccc7c8ccccc8sc7c65)c4)cc32)cc1 ; c1ccc(-n2c3ccccc3c3ccc([Si](c4ccccc4)(c4ccccc4)c4cccc(-n5c6ccccc6c6ccc7c8ccccc8n(-c8ccccc8)c7c65)c4)cc32)cc1 ; CC1(C)c2ccccc2-c2ccc3c4ccccc4n(-c4cccc([Si](c5ccccc5)(c5ccccc5)c5ccc6c7ccccc7n(-c7ccccc7)c6c5)c4)c3c21]

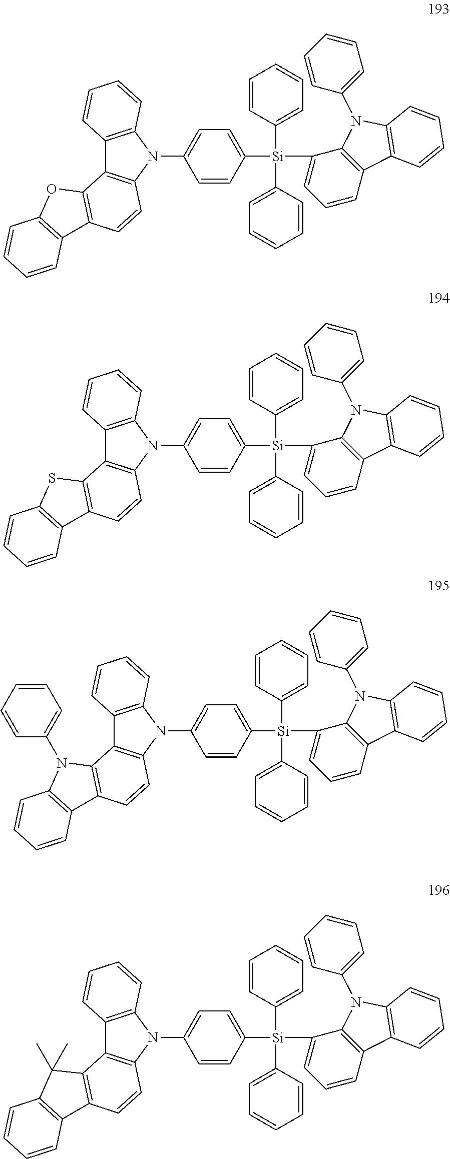 [SMILES: c1ccc(-n2c3ccccc3c3cccc([Si](c4ccccc4)(c4ccccc4)c4ccc(-n5c6ccccc6c6c7oc8ccccc8c7ccc65)cc4)c32)cc1 ; c1ccc(-n2c3ccccc3c3cccc([Si](c4ccccc4)(c4ccccc4)c4ccc(-n5c6ccccc6c6c7sc8ccccc8c7ccc65)cc4)c32)cc1 ; c1ccc(-n2c3ccccc3c3cccc([Si](c4ccccc4)(c4ccccc4)c4ccc(-n5c6ccccc6c6c5ccc5c7ccccc7n(-c7ccccc7)c56)cc4)c32)cc1 ; CC1(C)c2ccccc2-c2ccc3c(c21)c1ccccc1n3-c1ccc([Si](c2ccccc2)(c2ccccc2)c2cccc3c4ccccc4n(-c4ccccc4)c23)cc1]

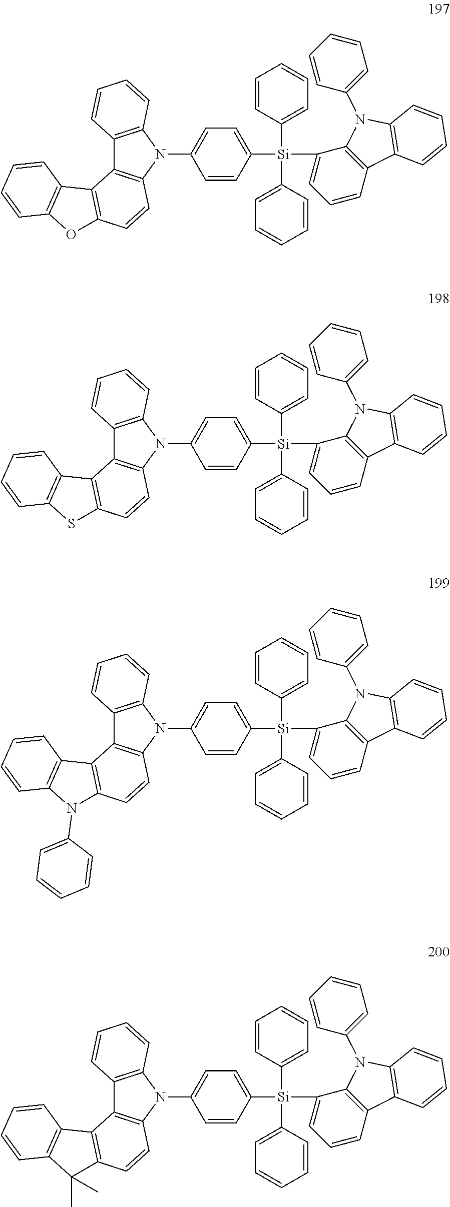 [SMILES: c1ccc(-n2c3ccccc3c3cccc([Si](c4ccccc4)(c4ccccc4)c4ccc(-n5c6ccccc6c6c7c(ccc65)oc5ccccc57)cc4)c32)cc1 ; c1ccc(-n2c3ccccc3c3cccc([Si](c4ccccc4)(c4ccccc4)c4ccc(-n5c6ccccc6c6c7c(ccc65)sc5ccccc57)cc4)c32)cc1 ; c1ccc(-n2c3ccccc3c3c4c5ccccc5n(-c5ccc([Si](c6ccccc6)(c6ccccc6)c6cccc7c8ccccc8n(-c8ccccc8)c67)cc5)c4ccc32)cc1 ; CC1(C)c2ccccc2-c2c1ccc1c2c2ccccc2n1-c1ccc([Si](c2ccccc2)(c2ccccc2)c2cccc3c4ccccc4n(-c4ccccc4)c23)cc1]

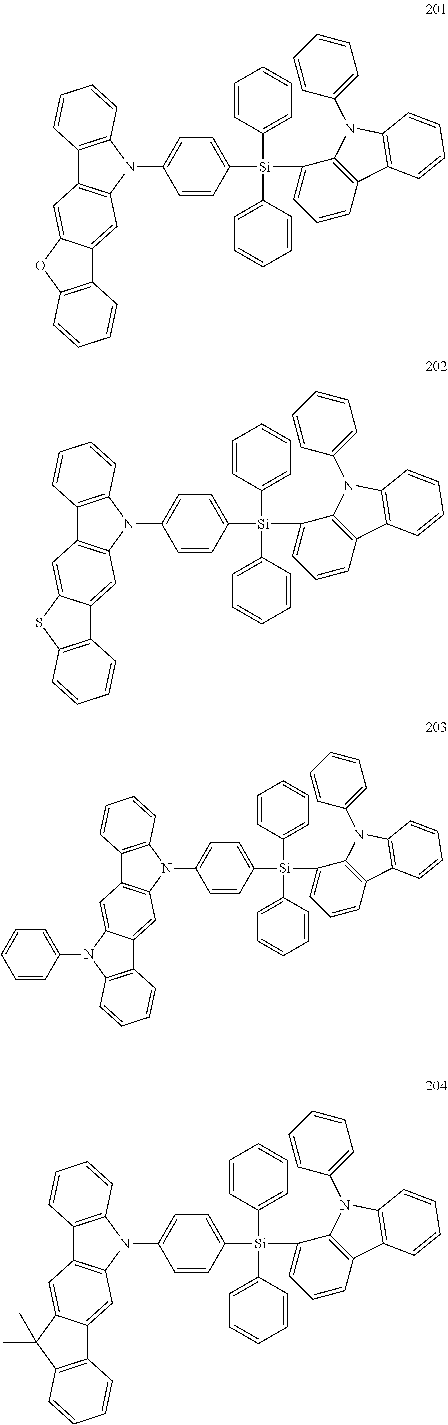 [SMILES: c1ccc(-n2c3ccccc3c3cccc([Si](c4ccccc4)(c4ccccc4)c4ccc(-n5c6ccccc6c6cc7oc8ccccc8c7cc65)cc4)c32)cc1 ; c1ccc(-n2c3ccccc3c3cccc([Si](c4ccccc4)(c4ccccc4)c4ccc(-n5c6ccccc6c6cc7sc8ccccc8c7cc65)cc4)c32)cc1 ; c1ccc(-n2c3ccccc3c3cc4c(cc32)c2ccccc2n4-c2ccc([Si](c3ccccc3)(c3ccccc3)c3cccc4c5ccccc5n(-c5ccccc5)c34)cc2)cc1 ; CC1(C)c2ccccc2-c2cc3c(cc21)c1ccccc1n3-c1ccc([Si](c2ccccc2)(c2ccccc2)c2cccc3c4ccccc4n(-c4ccccc4)c23)cc1]

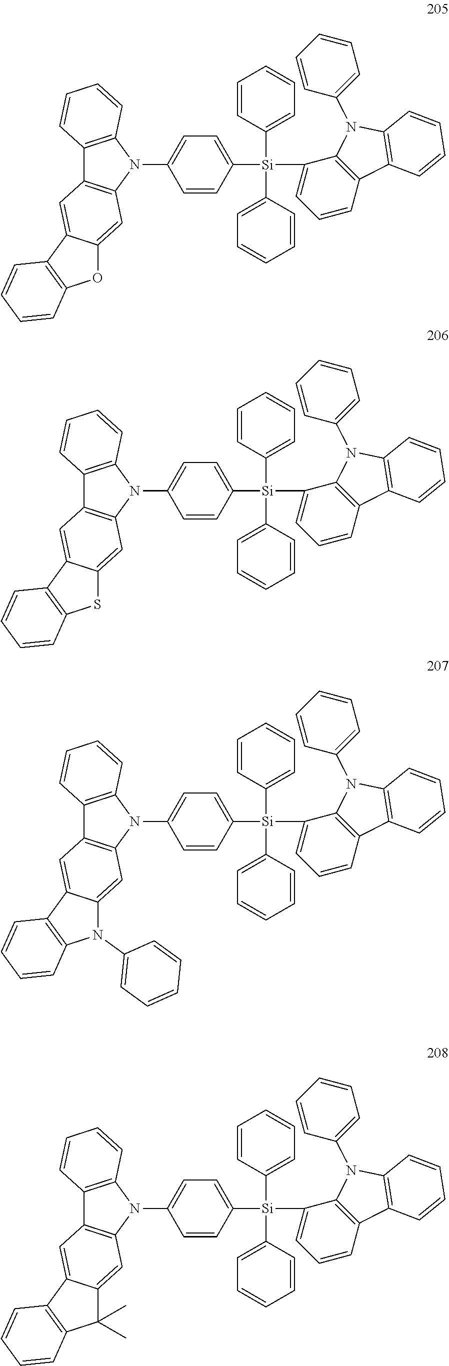 [SMILES: c1ccc(-n2c3ccccc3c3cccc([Si](c4ccccc4)(c4ccccc4)c4ccc(-n5c6ccccc6c6cc7c(cc65)oc5ccccc57)cc4)c32)cc1 ; c1ccc(-n2c3ccccc3c3cccc([Si](c4ccccc4)(c4ccccc4)c4ccc(-n5c6ccccc6c6cc7c(cc65)sc5ccccc57)cc4)c32)cc1 ; c1ccc(-n2c3ccccc3c3cc4c5ccccc5n(-c5ccc([Si](c6ccccc6)(c6ccccc6)c6cccc7c8ccccc8n(-c8ccccc8)c67)cc5)c4cc32)cc1 ; CC1(C)c2ccccc2-c2cc3c4ccccc4n(-c4ccc([Si](c5ccccc5)(c5ccccc5)c5cccc6c7ccccc7n(-c7ccccc7)c56)cc4)c3cc21]

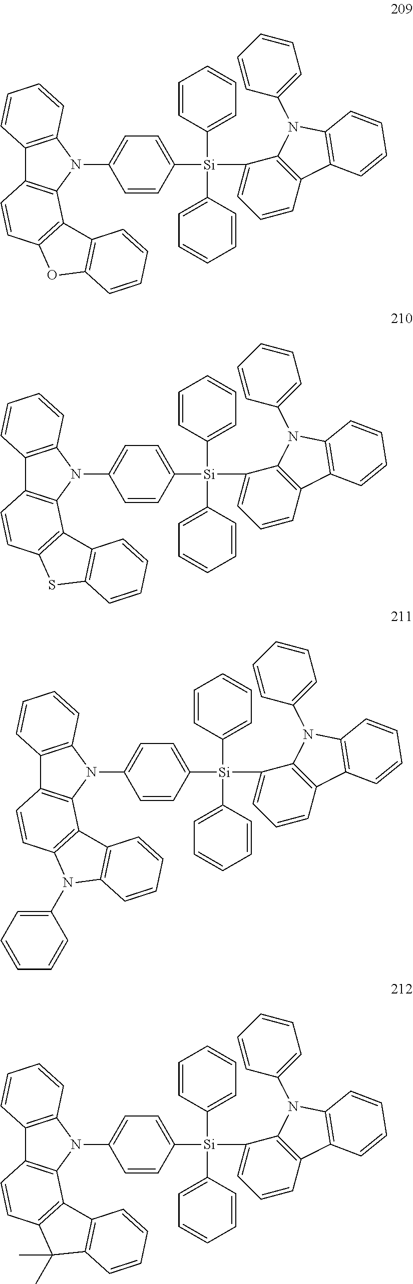 [SMILES: c1ccc(-n2c3ccccc3c3cccc([Si](c4ccccc4)(c4ccccc4)c4ccc(-n5c6ccccc6c6ccc7oc8ccccc8c7c65)cc4)c32)cc1 ; c1ccc(-n2c3ccccc3c3cccc([Si](c4ccccc4)(c4ccccc4)c4ccc(-n5c6ccccc6c6ccc7sc8ccccc8c7c65)cc4)c32)cc1 ; c1ccc(-n2c3ccccc3c3c2ccc2c4ccccc4n(-c4ccc([Si](c5ccccc5)(c5ccccc5)c5cccc6c7ccccc7n(-c7ccccc7)c56)cc4)c23)cc1 ; CC1(C)c2ccccc2-c2c1ccc1c3ccccc3n(-c3ccc([Si](c4ccccc4)(c4ccccc4)c4cccc5c6ccccc6n(-c6ccccc6)c45)cc3)c21]

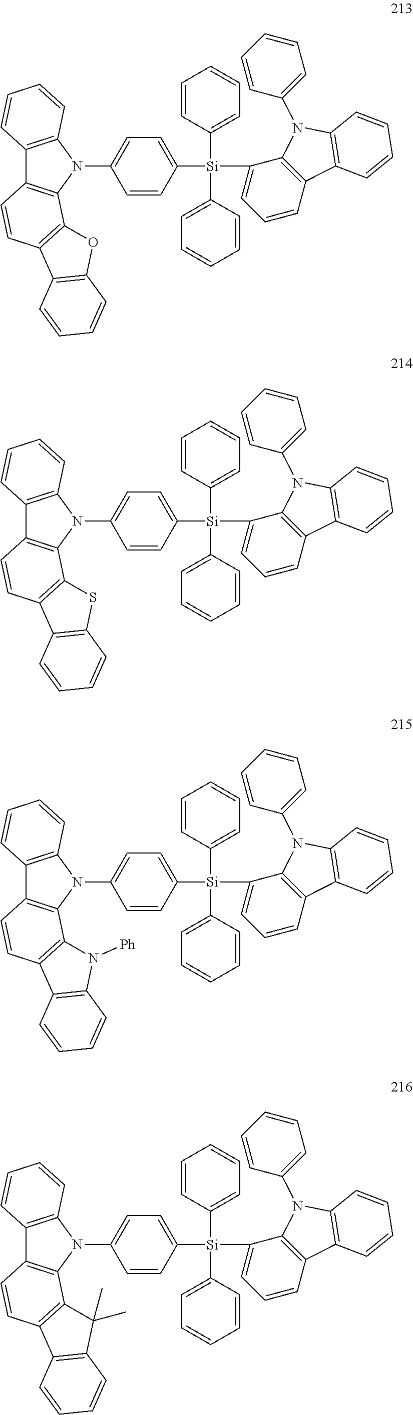 [SMILES: c1ccc(-n2c3ccccc3c3cccc([Si](c4ccccc4)(c4ccccc4)c4ccc(-n5c6ccccc6c6ccc7c8ccccc8oc7c65)cc4)c32)cc1 ; c1ccc(-n2c3ccccc3c3cccc([Si](c4ccccc4)(c4ccccc4)c4ccc(-n5c6ccccc6c6ccc7c8ccccc8sc7c65)cc4)c32)cc1 ; c1ccc(-n2c3ccccc3c3cccc([Si](c4ccccc4)(c4ccccc4)c4ccc(-n5c6ccccc6c6ccc7c8ccccc8n(-c8ccccc8)c7c65)cc4)c32)cc1 ; CC1(C)c2ccccc2-c2ccc3c4ccccc4n(-c4ccc([Si](c5ccccc5)(c5ccccc5)c5cccc6c7ccccc7n(-c7ccccc7)c56)cc4)c3c21]

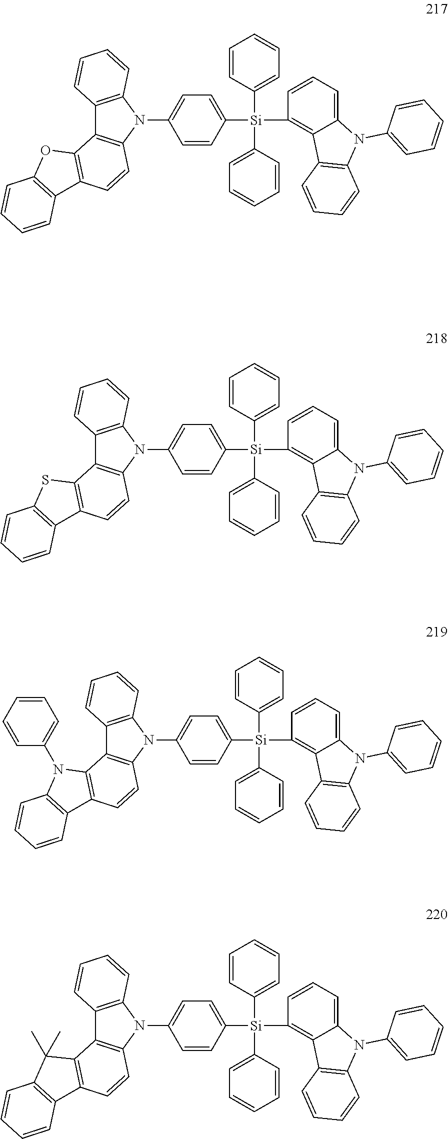 [SMILES: c1ccc(-n2c3ccccc3c3c([Si](c4ccccc4)(c4ccccc4)c4ccc(-n5c6ccccc6c6c7oc8ccccc8c7ccc65)cc4)cccc32)cc1 ; c1ccc(-n2c3ccccc3c3c([Si](c4ccccc4)(c4ccccc4)c4ccc(-n5c6ccccc6c6c7sc8ccccc8c7ccc65)cc4)cccc32)cc1 ; c1ccc(-n2c3ccccc3c3c([Si](c4ccccc4)(c4ccccc4)c4ccc(-n5c6ccccc6c6c5ccc5c7ccccc7n(-c7ccccc7)c56)cc4)cccc32)cc1 ; CC1(C)c2ccccc2-c2ccc3c(c21)c1ccccc1n3-c1ccc([Si](c2ccccc2)(c2ccccc2)c2cccc3c2c2ccccc2n3-c2ccccc2)cc1]

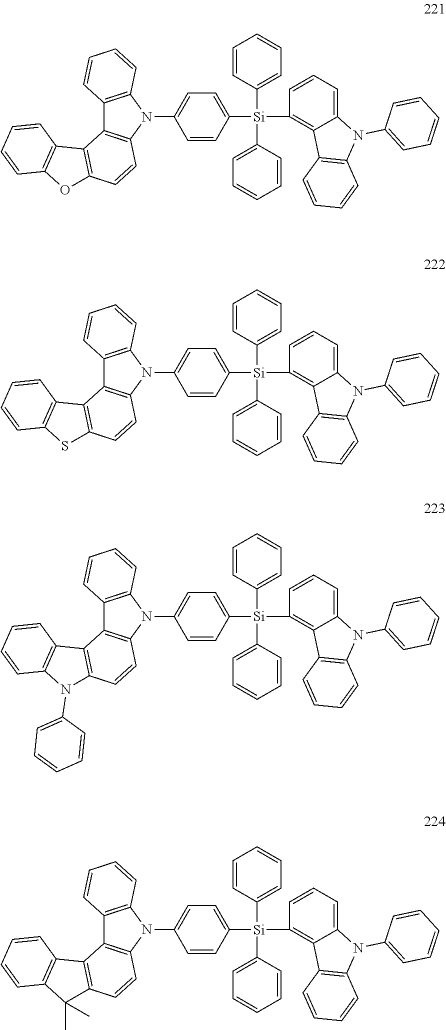 [SMILES: c1ccc(-n2c3ccccc3c3c([Si](c4ccccc4)(c4ccccc4)c4ccc(-n5c6ccccc6c6c7c(ccc65)oc5ccccc57)cc4)cccc32)cc1 ; c1ccc(-n2c3ccccc3c3c([Si](c4ccccc4)(c4ccccc4)c4ccc(-n5c6ccccc6c6c7c(ccc65)sc5ccccc57)cc4)cccc32)cc1 ; c1ccc(-n2c3ccccc3c3c([Si](c4ccccc4)(c4ccccc4)c4ccc(-n5c6ccccc6c6c7c8ccccc8n(-c8ccccc8)c7ccc65)cc4)cccc32)cc1 ; CC1(C)c2ccccc2-c2c1ccc1c2c2ccccc2n1-c1ccc([Si](c2ccccc2)(c2ccccc2)c2cccc3c2c2ccccc2n3-c2ccccc2)cc1]

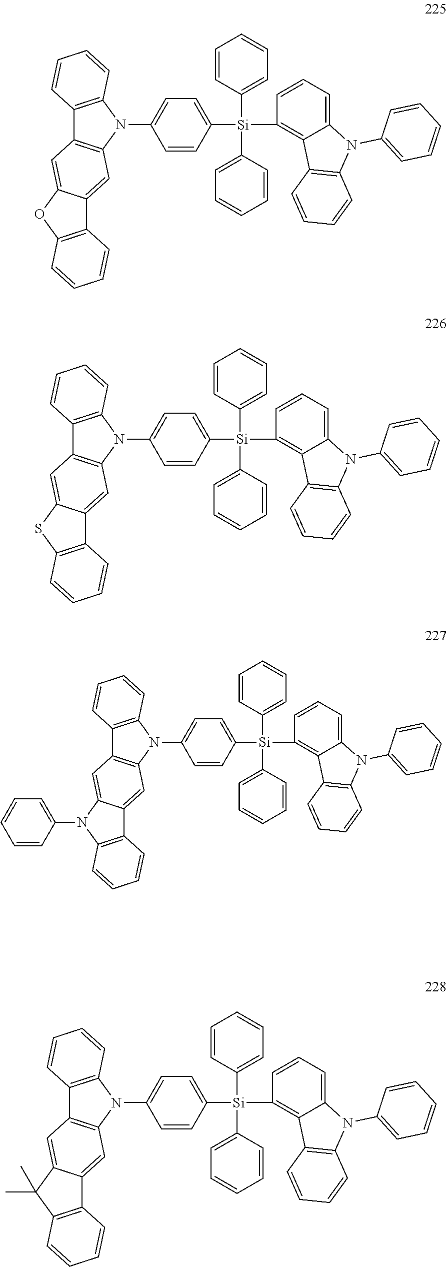 [SMILES: c1ccc(-n2c3ccccc3c3c([Si](c4ccccc4)(c4ccccc4)c4ccc(-n5c6ccccc6c6cc7oc8ccccc8c7cc65)cc4)cccc32)cc1 ; c1ccc(-n2c3ccccc3c3c([Si](c4ccccc4)(c4ccccc4)c4ccc(-n5c6ccccc6c6cc7sc8ccccc8c7cc65)cc4)cccc32)cc1 ; c1ccc(-n2c3ccccc3c3cc4c(cc32)c2ccccc2n4-c2ccc([Si](c3ccccc3)(c3ccccc3)c3cccc4c3c3ccccc3n4-c3ccccc3)cc2)cc1 ; CC1(C)c2ccccc2-c2cc3c(cc21)c1ccccc1n3-c1ccc([Si](c2ccccc2)(c2ccccc2)c2cccc3c2c2ccccc2n3-c2ccccc2)cc1]

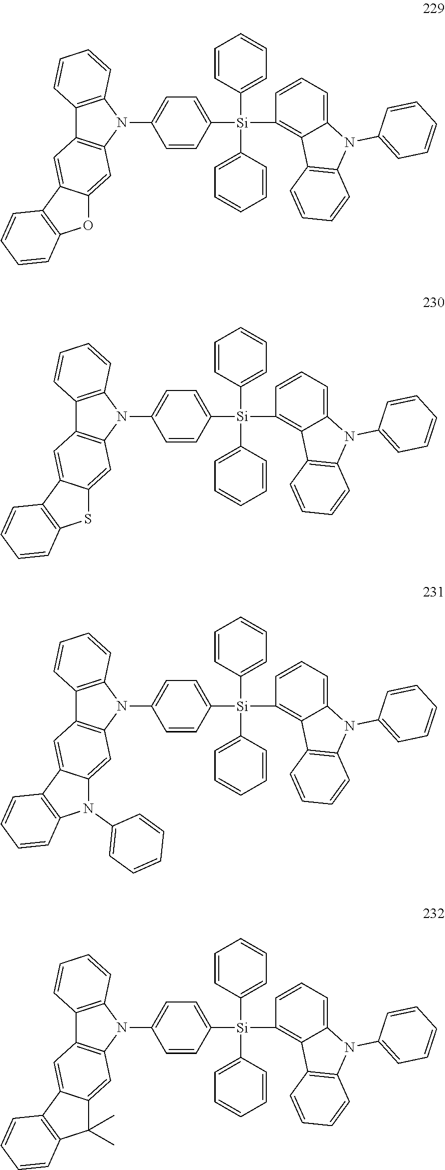 [SMILES: c1ccc(-n2c3ccccc3c3c([Si](c4ccccc4)(c4ccccc4)c4ccc(-n5c6ccccc6c6cc7c(cc65)oc5ccccc57)cc4)cccc32)cc1 ; c1ccc(-n2c3ccccc3c3c([Si](c4ccccc4)(c4ccccc4)c4ccc(-n5c6ccccc6c6cc7c(cc65)sc5ccccc57)cc4)cccc32)cc1 ; c1ccc(-n2c3ccccc3c3cc4c5ccccc5n(-c5ccc([Si](c6ccccc6)(c6ccccc6)c6cccc7c6c6ccccc6n7-c6ccccc6)cc5)c4cc32)cc1 ; CC1(C)c2ccccc2-c2cc3c4ccccc4n(-c4ccc([Si](c5ccccc5)(c5ccccc5)c5cccc6c5c5ccccc5n6-c5ccccc5)cc4)c3cc21]

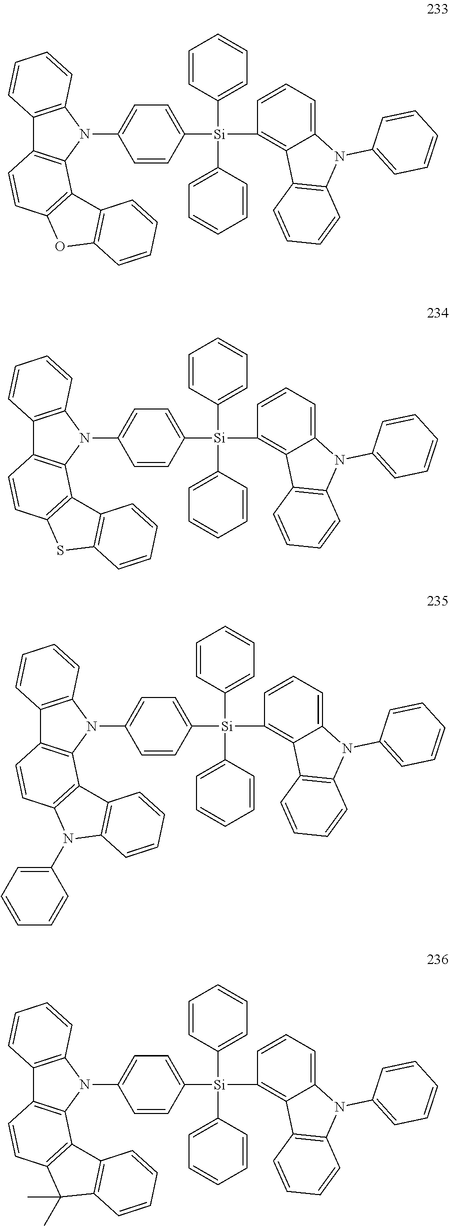 [SMILES: c1ccc(-n2c3ccccc3c3c([Si](c4ccccc4)(c4ccccc4)c4ccc(-n5c6ccccc6c6ccc7oc8ccccc8c7c65)cc4)cccc32)cc1 ; c1ccc(-n2c3ccccc3c3c([Si](c4ccccc4)(c4ccccc4)c4ccc(-n5c6ccccc6c6ccc7sc8ccccc8c7c65)cc4)cccc32)cc1 ; c1ccc(-n2c3ccccc3c3c([Si](c4ccccc4)(c4ccccc4)c4ccc(-n5c6ccccc6c6ccc7c(c8ccccc8n7-c7ccccc7)c65)cc4)cccc32)cc1 ; CC1(C)c2ccccc2-c2c1ccc1c3ccccc3n(-c3ccc([Si](c4ccccc4)(c4ccccc4)c4cccc5c4c4ccccc4n5-c4ccccc4)cc3)c21]

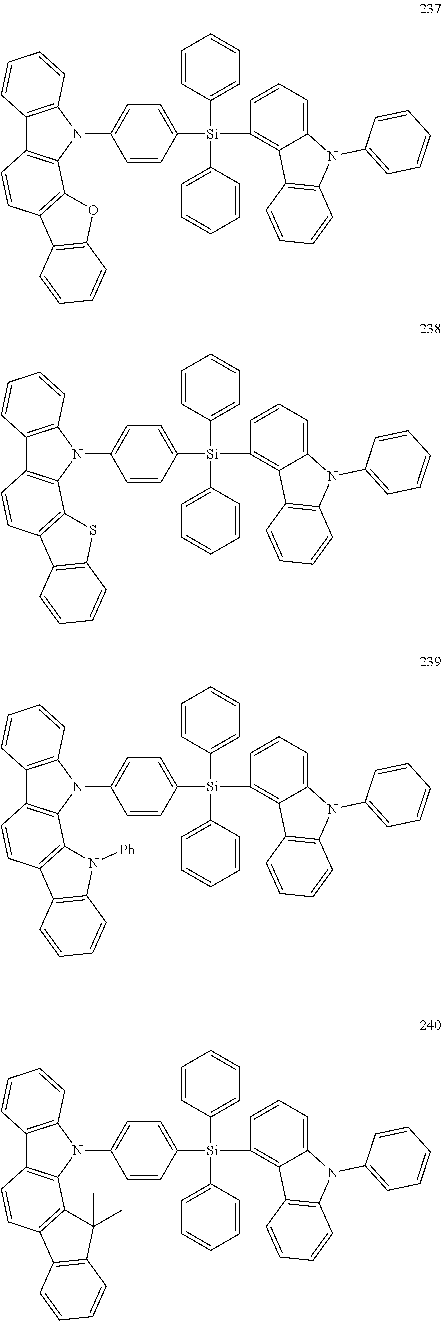 [SMILES: c1ccc(-n2c3ccccc3c3c([Si](c4ccccc4)(c4ccccc4)c4ccc(-n5c6ccccc6c6ccc7c8ccccc8oc7c65)cc4)cccc32)cc1 ; c1ccc(-n2c3ccccc3c3c([Si](c4ccccc4)(c4ccccc4)c4ccc(-n5c6ccccc6c6ccc7c8ccccc8sc7c65)cc4)cccc32)cc1 ; c1ccc(-n2c3ccccc3c3c([Si](c4ccccc4)(c4ccccc4)c4ccc(-n5c6ccccc6c6ccc7c8ccccc8n(-c8ccccc8)c7c65)cc4)cccc32)cc1 ; CC1(C)c2ccccc2-c2ccc3c4ccccc4n(-c4ccc([Si](c5ccccc5)(c5ccccc5)c5cccc6c5c5ccccc5n6-c5ccccc5)cc4)c3c21]

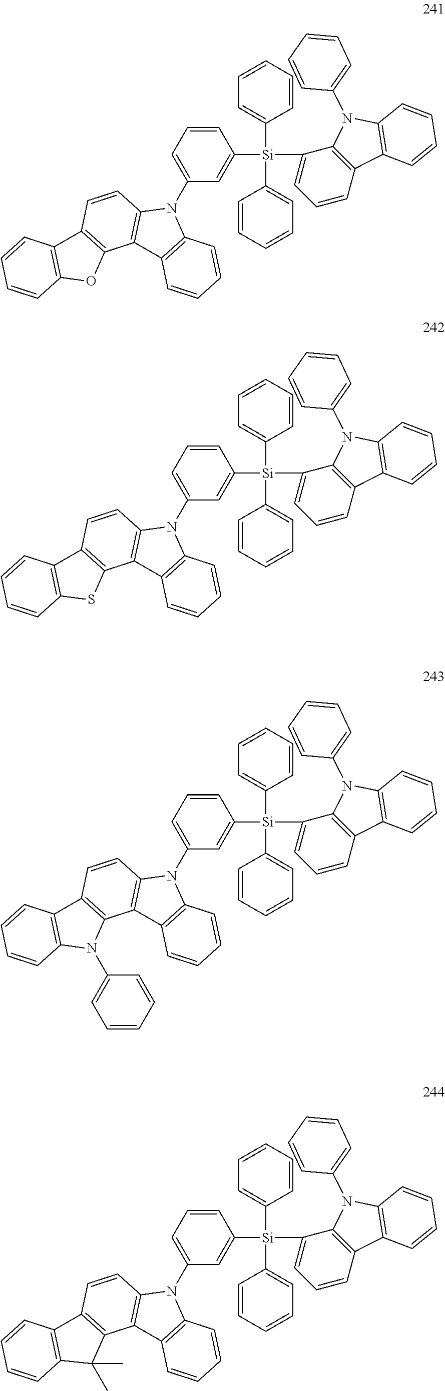 [SMILES: c1ccc(-n2c3ccccc3c3cccc([Si](c4ccccc4)(c4ccccc4)c4cccc(-n5c6ccccc6c6c7oc8ccccc8c7ccc65)c4)c32)cc1 ; c1ccc(-n2c3ccccc3c3cccc([Si](c4ccccc4)(c4ccccc4)c4cccc(-n5c6ccccc6c6c7sc8ccccc8c7ccc65)c4)c32)cc1 ; c1ccc(-n2c3ccccc3c3cccc([Si](c4ccccc4)(c4ccccc4)c4cccc(-n5c6ccccc6c6c5ccc5c7ccccc7n(-c7ccccc7)c56)c4)c32)cc1 ; CC1(C)c2ccccc2-c2ccc3c(c21)c1ccccc1n3-c1cccc([Si](c2ccccc2)(c2ccccc2)c2cccc3c4ccccc4n(-c4ccccc4)c23)c1]

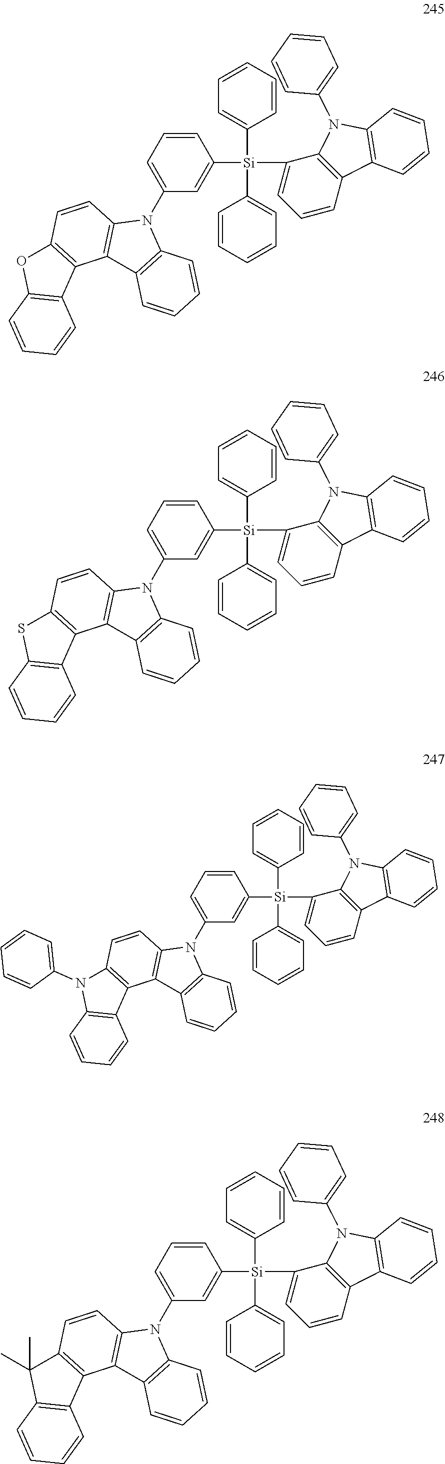 [SMILES: c1ccc(-n2c3ccccc3c3cccc([Si](c4ccccc4)(c4ccccc4)c4cccc(-n5c6ccccc6c6c7c(ccc65)oc5ccccc57)c4)c32)cc1 ; c1ccc(-n2c3ccccc3c3cccc([Si](c4ccccc4)(c4ccccc4)c4cccc(-n5c6ccccc6c6c7c(ccc65)sc5ccccc57)c4)c32)cc1 ; c1ccc(-n2c3ccccc3c3c4c5ccccc5n(-c5cccc([Si](c6ccccc6)(c6ccccc6)c6cccc7c8ccccc8n(-c8ccccc8)c67)c5)c4ccc32)cc1 ; CC1(C)c2ccccc2-c2c1ccc1c2c2ccccc2n1-c1cccc([Si](c2ccccc2)(c2ccccc2)c2cccc3c4ccccc4n(-c4ccccc4)c23)c1]

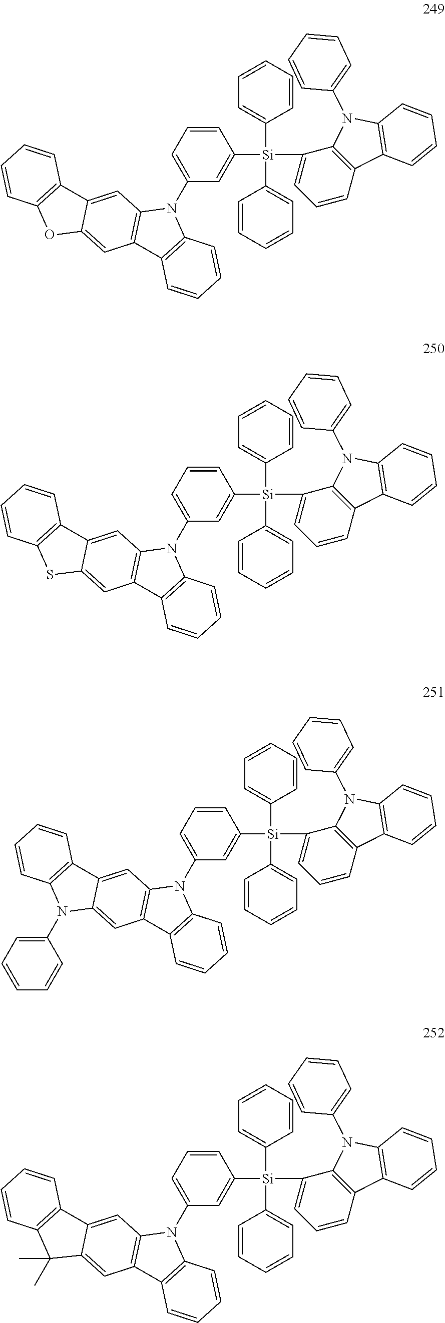 [SMILES: c1ccc(-n2c3ccccc3c3cccc([Si](c4ccccc4)(c4ccccc4)c4cccc(-n5c6ccccc6c6cc7oc8ccccc8c7cc65)c4)c32)cc1 ; c1ccc(-n2c3ccccc3c3cccc([Si](c4ccccc4)(c4ccccc4)c4cccc(-n5c6ccccc6c6cc7sc8ccccc8c7cc65)c4)c32)cc1 ; c1ccc(-n2c3ccccc3c3cc4c(cc32)c2ccccc2n4-c2cccc([Si](c3ccccc3)(c3ccccc3)c3cccc4c5ccccc5n(-c5ccccc5)c34)c2)cc1 ; CC1(C)c2ccccc2-c2cc3c(cc21)c1ccccc1n3-c1cccc([Si](c2ccccc2)(c2ccccc2)c2cccc3c4ccccc4n(-c4ccccc4)c23)c1]

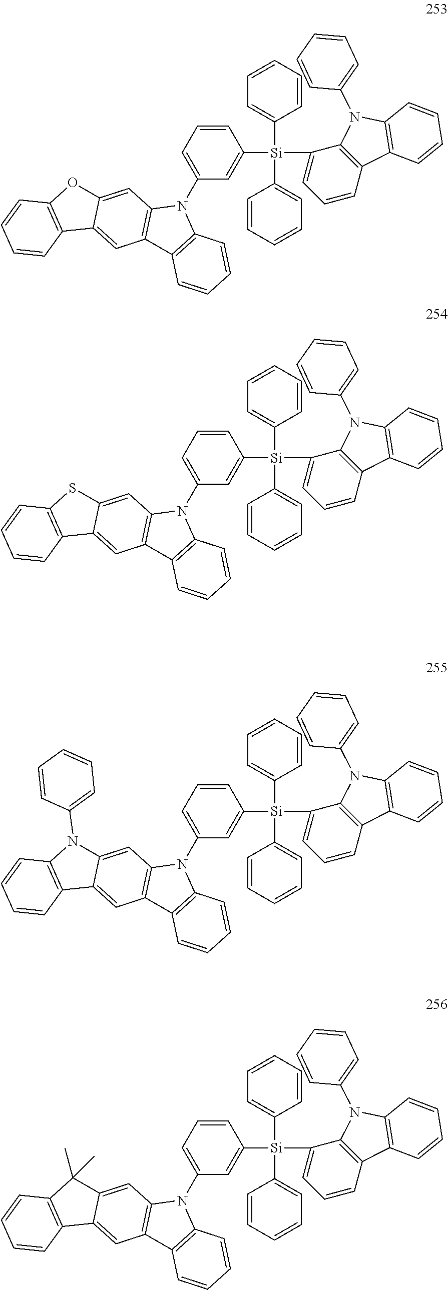 [SMILES: c1ccc(-n2c3ccccc3c3cccc([Si](c4ccccc4)(c4ccccc4)c4cccc(-n5c6ccccc6c6cc7c(cc65)oc5ccccc57)c4)c32)cc1 ; c1ccc(-n2c3ccccc3c3cccc([Si](c4ccccc4)(c4ccccc4)c4cccc(-n5c6ccccc6c6cc7c(cc65)sc5ccccc57)c4)c32)cc1 ; c1ccc(-n2c3ccccc3c3cc4c5ccccc5n(-c5cccc([Si](c6ccccc6)(c6ccccc6)c6cccc7c8ccccc8n(-c8ccccc8)c67)c5)c4cc32)cc1 ; CC1(C)c2ccccc2-c2cc3c4ccccc4n(-c4cccc([Si](c5ccccc5)(c5ccccc5)c5cccc6c7ccccc7n(-c7ccccc7)c56)c4)c3cc21]

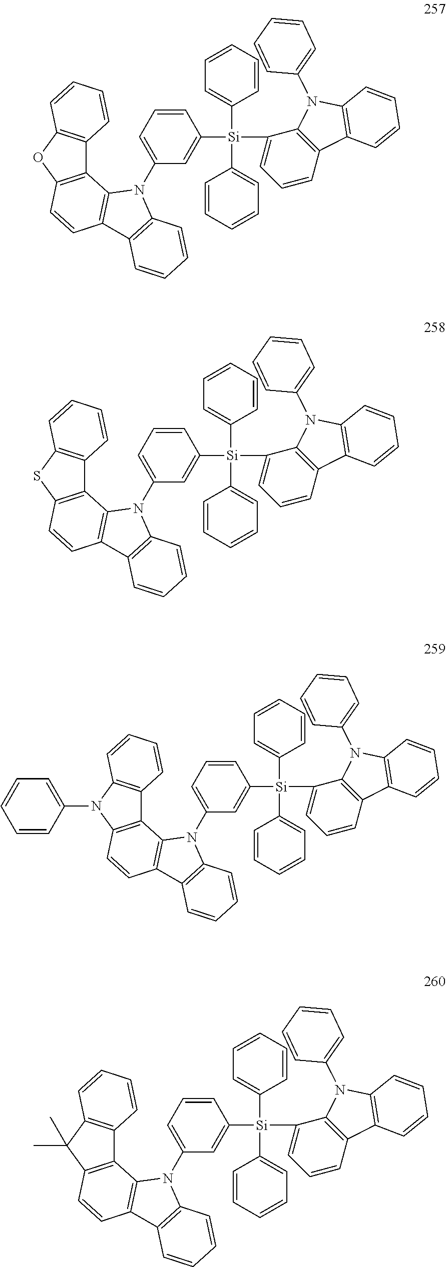 [SMILES: c1ccc(-n2c3ccccc3c3cccc([Si](c4ccccc4)(c4ccccc4)c4cccc(-n5c6ccccc6c6ccc7oc8ccccc8c7c65)c4)c32)cc1 ; c1ccc(-n2c3ccccc3c3cccc([Si](c4ccccc4)(c4ccccc4)c4cccc(-n5c6ccccc6c6ccc7sc8ccccc8c7c65)c4)c32)cc1 ; c1ccc(-n2c3ccccc3c3c2ccc2c4ccccc4n(-c4cccc([Si](c5ccccc5)(c5ccccc5)c5cccc6c7ccccc7n(-c7ccccc7)c56)c4)c23)cc1 ; CC1(C)c2ccccc2-c2c1ccc1c3ccccc3n(-c3cccc([Si](c4ccccc4)(c4ccccc4)c4cccc5c6ccccc6n(-c6ccccc6)c45)c3)c21]

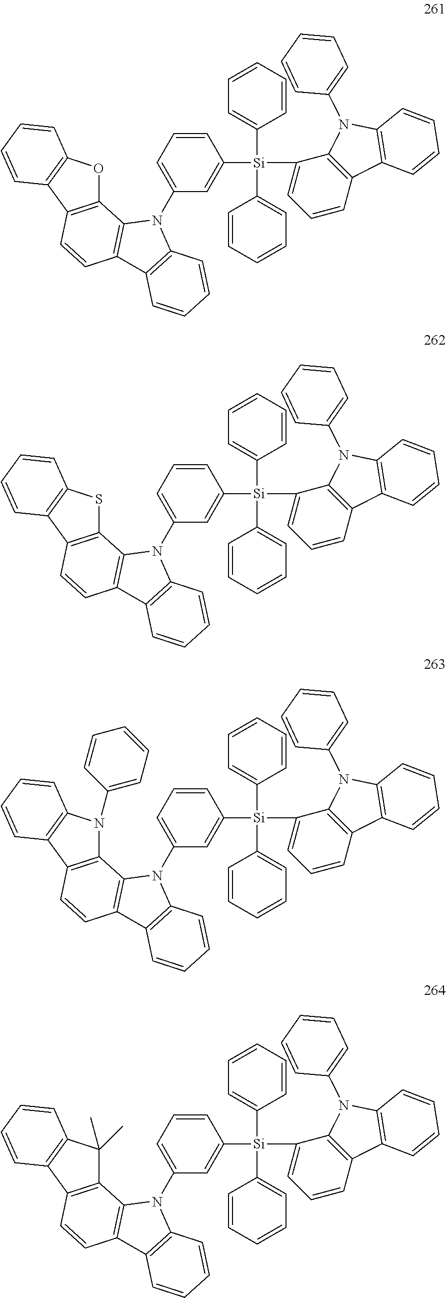 [SMILES: c1ccc(-n2c3ccccc3c3cccc([Si](c4ccccc4)(c4ccccc4)c4cccc(-n5c6ccccc6c6ccc7c8ccccc8oc7c65)c4)c32)cc1 ; c1ccc(-n2c3ccccc3c3cccc([Si](c4ccccc4)(c4ccccc4)c4cccc(-n5c6ccccc6c6ccc7c8ccccc8sc7c65)c4)c32)cc1 ; c1ccc(-n2c3ccccc3c3cccc([Si](c4ccccc4)(c4ccccc4)c4cccc(-n5c6ccccc6c6ccc7c8ccccc8n(-c8ccccc8)c7c65)c4)c32)cc1 ; CC1(C)c2ccccc2-c2ccc3c4ccccc4n(-c4cccc([Si](c5ccccc5)(c5ccccc5)c5cccc6c7ccccc7n(-c7ccccc7)c56)c4)c3c21]

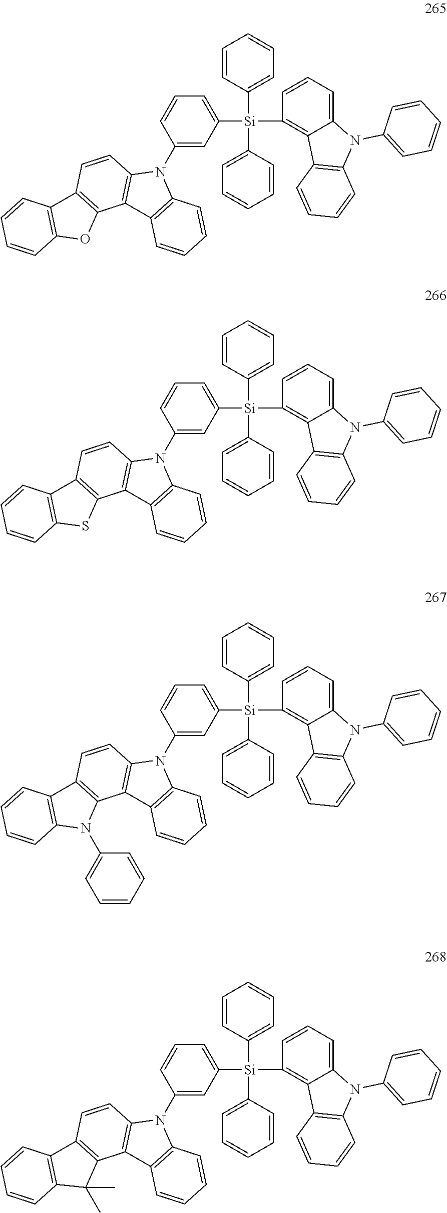 [SMILES: c1ccc(-n2c3ccccc3c3c([Si](c4ccccc4)(c4ccccc4)c4cccc(-n5c6ccccc6c6c7oc8ccccc8c7ccc65)c4)cccc32)cc1 ; c1ccc(-n2c3ccccc3c3c([Si](c4ccccc4)(c4ccccc4)c4cccc(-n5c6ccccc6c6c7sc8ccccc8c7ccc65)c4)cccc32)cc1 ; c1ccc(-n2c3ccccc3c3c([Si](c4ccccc4)(c4ccccc4)c4cccc(-n5c6ccccc6c6c5ccc5c7ccccc7n(-c7ccccc7)c56)c4)cccc32)cc1 ; CC1(C)c2ccccc2-c2ccc3c(c21)c1ccccc1n3-c1cccc([Si](c2ccccc2)(c2ccccc2)c2cccc3c2c2ccccc2n3-c2ccccc2)c1]

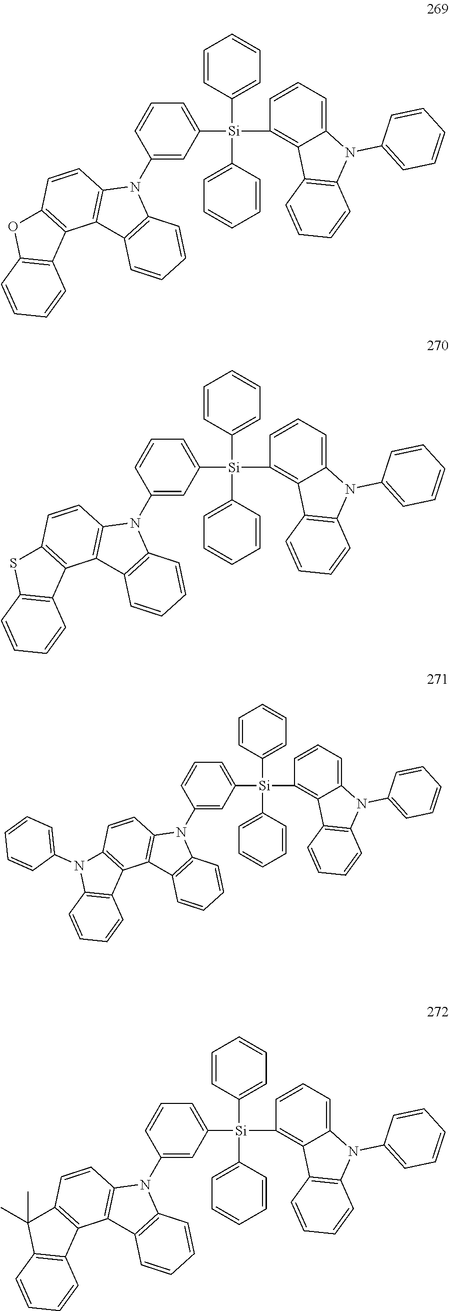 [SMILES: c1ccc(-n2c3ccccc3c3c([Si](c4ccccc4)(c4ccccc4)c4cccc(-n5c6ccccc6c6c7c(ccc65)oc5ccccc57)c4)cccc32)cc1 ; c1ccc(-n2c3ccccc3c3c([Si](c4ccccc4)(c4ccccc4)c4cccc(-n5c6ccccc6c6c7c(ccc65)sc5ccccc57)c4)cccc32)cc1 ; c1ccc(-n2c3ccccc3c3c([Si](c4ccccc4)(c4ccccc4)c4cccc(-n5c6ccccc6c6c7c8ccccc8n(-c8ccccc8)c7ccc65)c4)cccc32)cc1 ; CC1(C)c2ccccc2-c2c1ccc1c2c2ccccc2n1-c1cccc([Si](c2ccccc2)(c2ccccc2)c2cccc3c2c2ccccc2n3-c2ccccc2)c1]

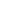

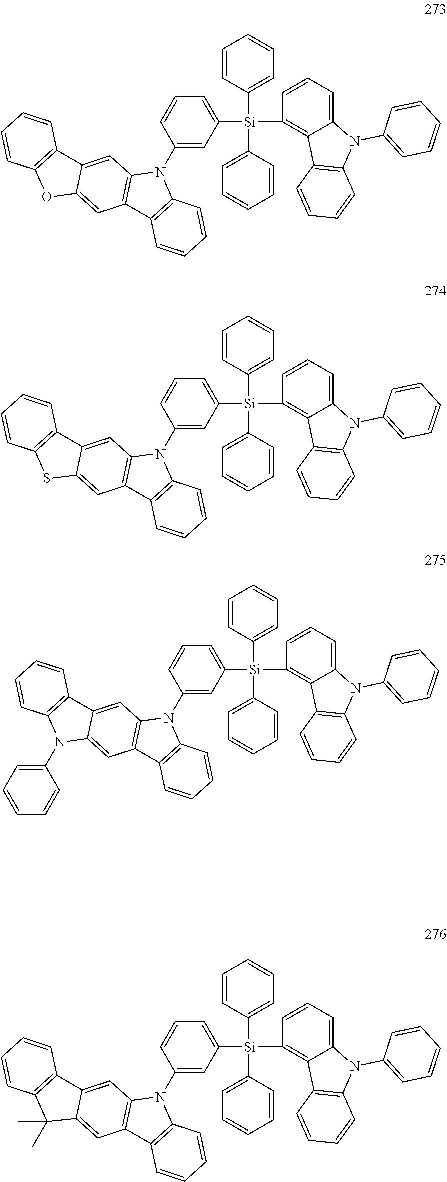 [SMILES: c1ccc(-n2c3ccccc3c3c([Si](c4ccccc4)(c4ccccc4)c4cccc(-n5c6ccccc6c6cc7oc8ccccc8c7cc65)c4)cccc32)cc1 ; c1ccc(-n2c3ccccc3c3c([Si](c4ccccc4)(c4ccccc4)c4cccc(-n5c6ccccc6c6cc7sc8ccccc8c7cc65)c4)cccc32)cc1 ; c1ccc(-n2c3ccccc3c3cc4c(cc32)c2ccccc2n4-c2cccc([Si](c3ccccc3)(c3ccccc3)c3cccc4c3c3ccccc3n4-c3ccccc3)c2)cc1 ; CC1(C)c2ccccc2-c2cc3c(cc21)c1ccccc1n3-c1cccc([Si](c2ccccc2)(c2ccccc2)c2cccc3c2c2ccccc2n3-c2ccccc2)c1]

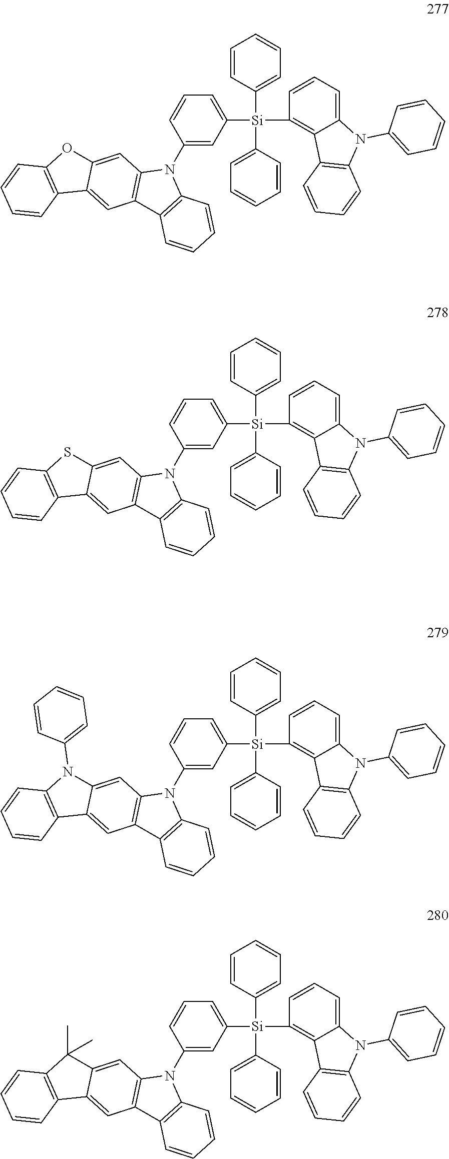 [SMILES: c1ccc(-n2c3ccccc3c3c([Si](c4ccccc4)(c4ccccc4)c4cccc(-n5c6ccccc6c6cc7c(cc65)oc5ccccc57)c4)cccc32)cc1 ; c1ccc(-n2c3ccccc3c3c([Si](c4ccccc4)(c4ccccc4)c4cccc(-n5c6ccccc6c6cc7c(cc65)sc5ccccc57)c4)cccc32)cc1 ; c1ccc(-n2c3ccccc3c3cc4c5ccccc5n(-c5cccc([Si](c6ccccc6)(c6ccccc6)c6cccc7c6c6ccccc6n7-c6ccccc6)c5)c4cc32)cc1 ; CC1(C)c2ccccc2-c2cc3c4ccccc4n(-c4cccc([Si](c5ccccc5)(c5ccccc5)c5cccc6c5c5ccccc5n6-c5ccccc5)c4)c3cc21]

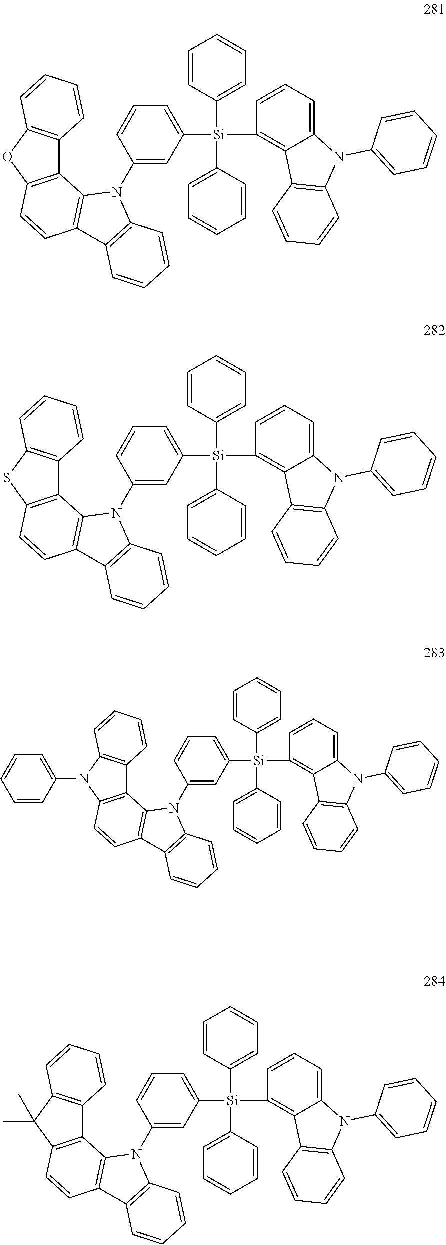 [SMILES: c1ccc(-n2c3ccccc3c3c([Si](c4ccccc4)(c4ccccc4)c4cccc(-n5c6ccccc6c6ccc7oc8ccccc8c7c65)c4)cccc32)cc1 ; c1ccc(-n2c3ccccc3c3c([Si](c4ccccc4)(c4ccccc4)c4cccc(-n5c6ccccc6c6ccc7sc8ccccc8c7c65)c4)cccc32)cc1 ; c1ccc(-n2c3ccccc3c3c([Si](c4ccccc4)(c4ccccc4)c4cccc(-n5c6ccccc6c6ccc7c(c8ccccc8n7-c7ccccc7)c65)c4)cccc32)cc1 ; CC1(C)c2ccccc2-c2c1ccc1c3ccccc3n(-c3cccc([Si](c4ccccc4)(c4ccccc4)c4cccc5c4c4ccccc4n5-c4ccccc4)c3)c21]

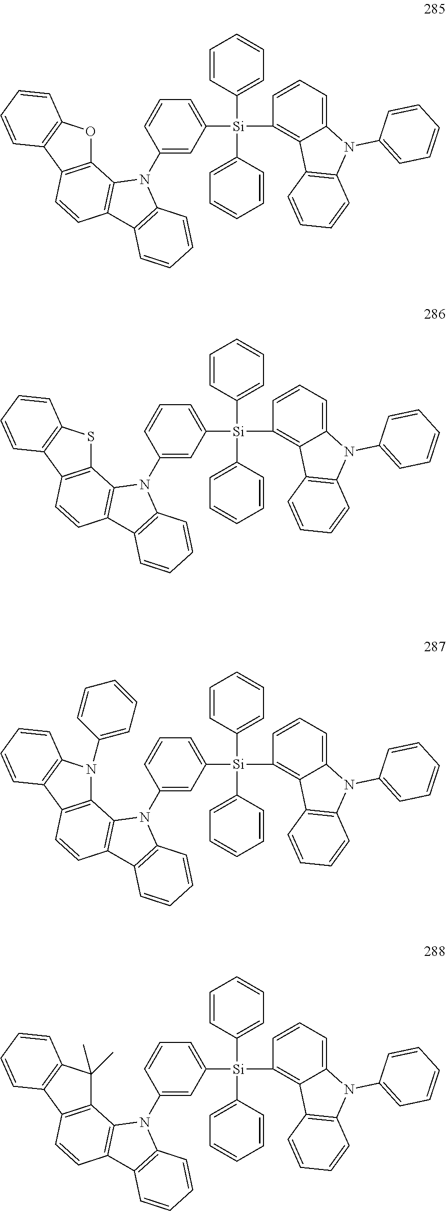 [SMILES: c1ccc(-n2c3ccccc3c3c([Si](c4ccccc4)(c4ccccc4)c4cccc(-n5c6ccccc6c6ccc7c8ccccc8oc7c65)c4)cccc32)cc1 ; c1ccc(-n2c3ccccc3c3c([Si](c4ccccc4)(c4ccccc4)c4cccc(-n5c6ccccc6c6ccc7c8ccccc8sc7c65)c4)cccc32)cc1 ; c1ccc(-n2c3ccccc3c3c([Si](c4ccccc4)(c4ccccc4)c4cccc(-n5c6ccccc6c6ccc7c8ccccc8n(-c8ccccc8)c7c65)c4)cccc32)cc1 ; CC1(C)c2ccccc2-c2ccc3c4ccccc4n(-c4cccc([Si](c5ccccc5)(c5ccccc5)c5cccc6c5c5ccccc5n6-c5ccccc5)c4)c3c21]

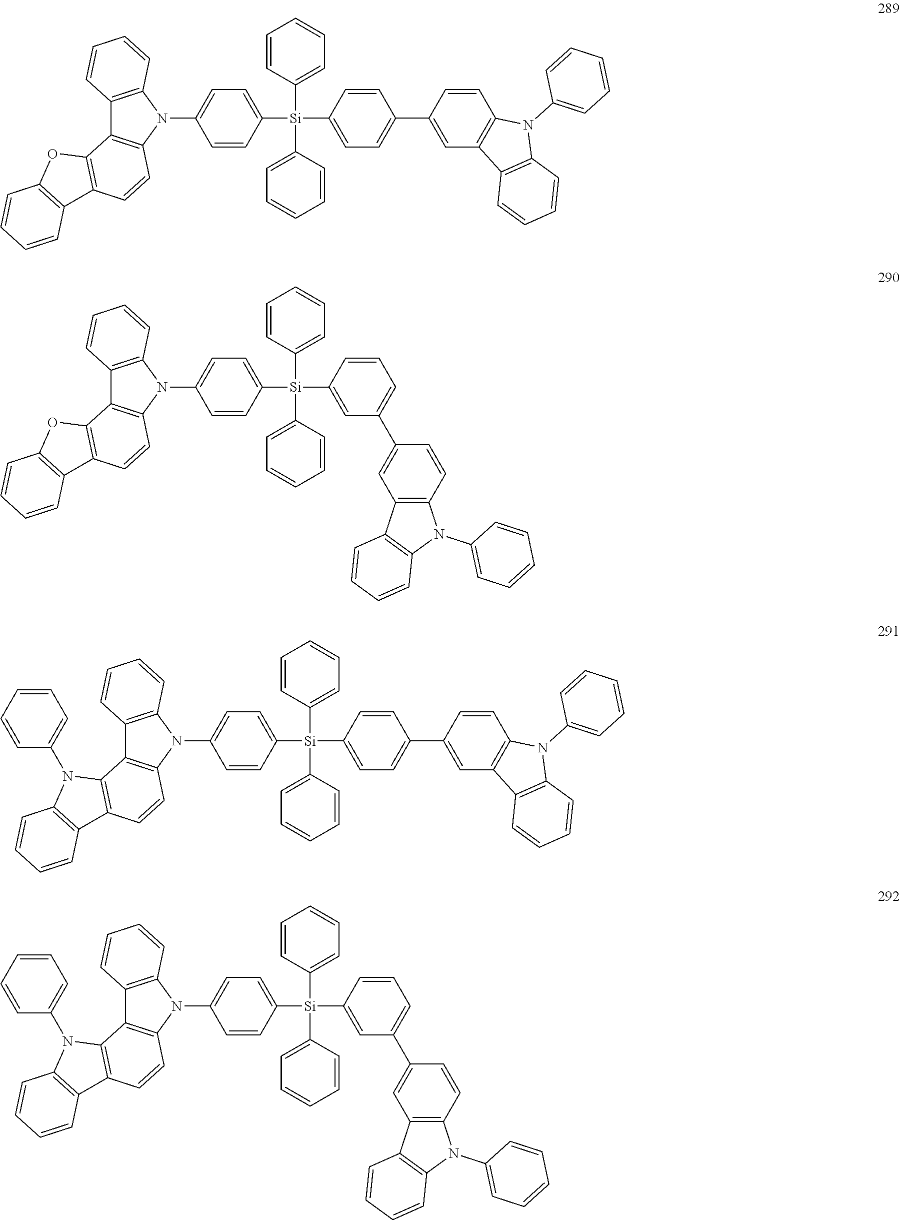 [SMILES: c1ccc(-n2c3ccccc3c3cc(-c4ccc([Si](c5ccccc5)(c5ccccc5)c5ccc(-n6c7ccccc7c7c8oc9ccccc9c8ccc76)cc5)cc4)ccc32)cc1 ; c1ccc(-n2c3ccccc3c3cc(-c4cccc([Si](c5ccccc5)(c5ccccc5)c5ccc(-n6c7ccccc7c7c8oc9ccccc9c8ccc76)cc5)c4)ccc32)cc1 ; c1ccc(-n2c3ccccc3c3cc(-c4ccc([Si](c5ccccc5)(c5ccccc5)c5ccc(-n6c7ccccc7c7c6ccc6c8ccccc8n(-c8ccccc8)c67)cc5)cc4)ccc32)cc1 ; c1ccc(-n2c3ccccc3c3cc(-c4cccc([Si](c5ccccc5)(c5ccccc5)c5ccc(-n6c7ccccc7c7c6ccc6c8ccccc8n(-c8ccccc8)c67)cc5)c4)ccc32)cc1]

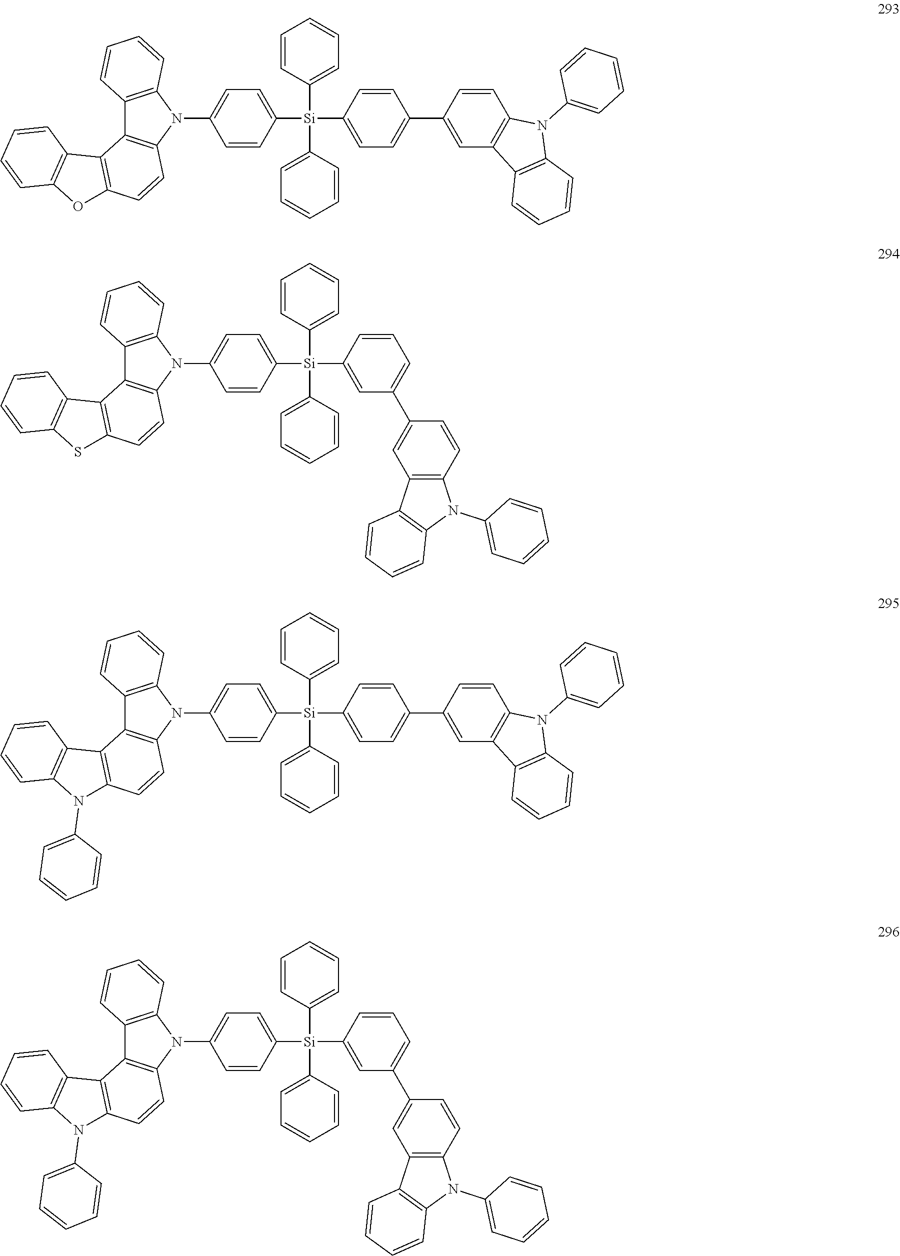

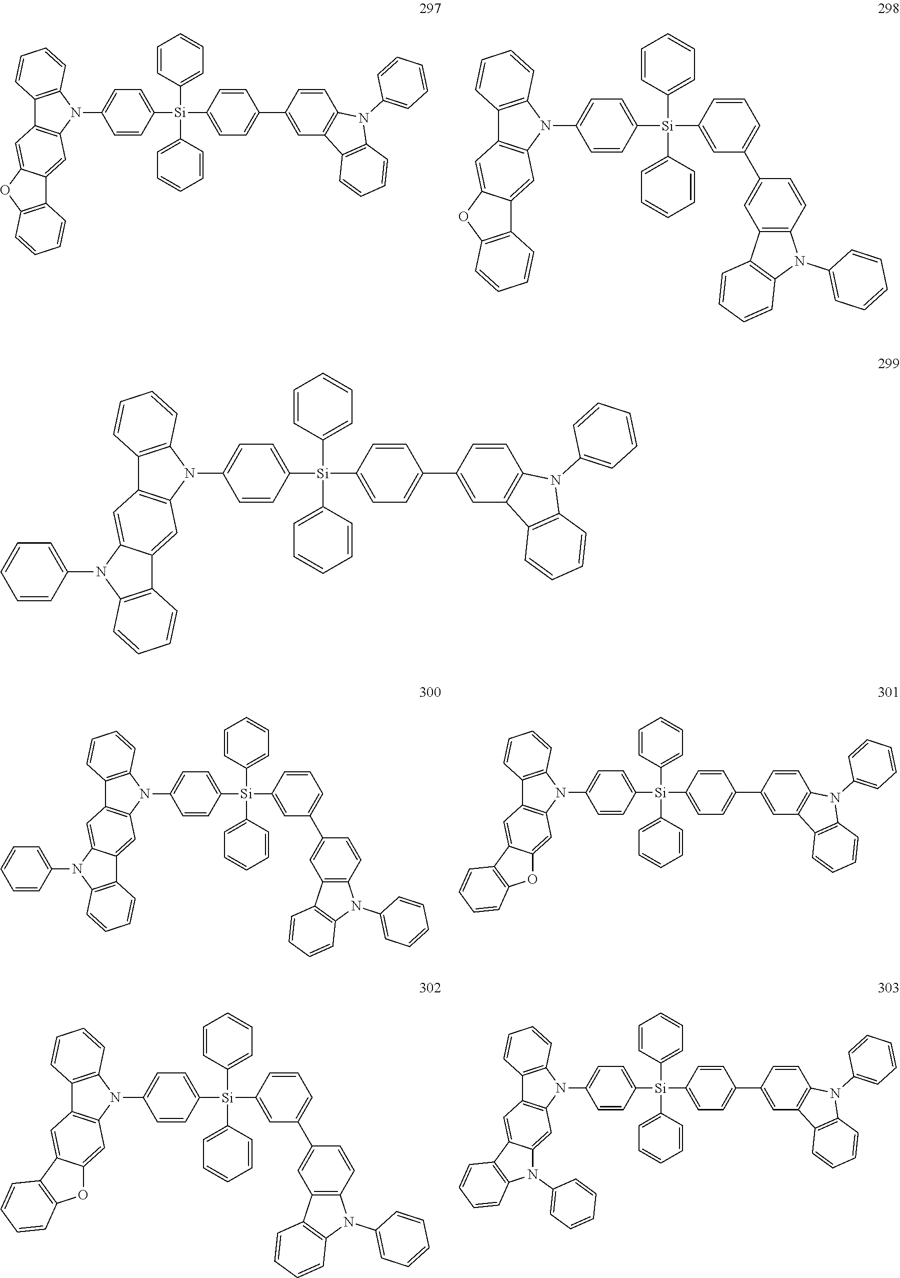

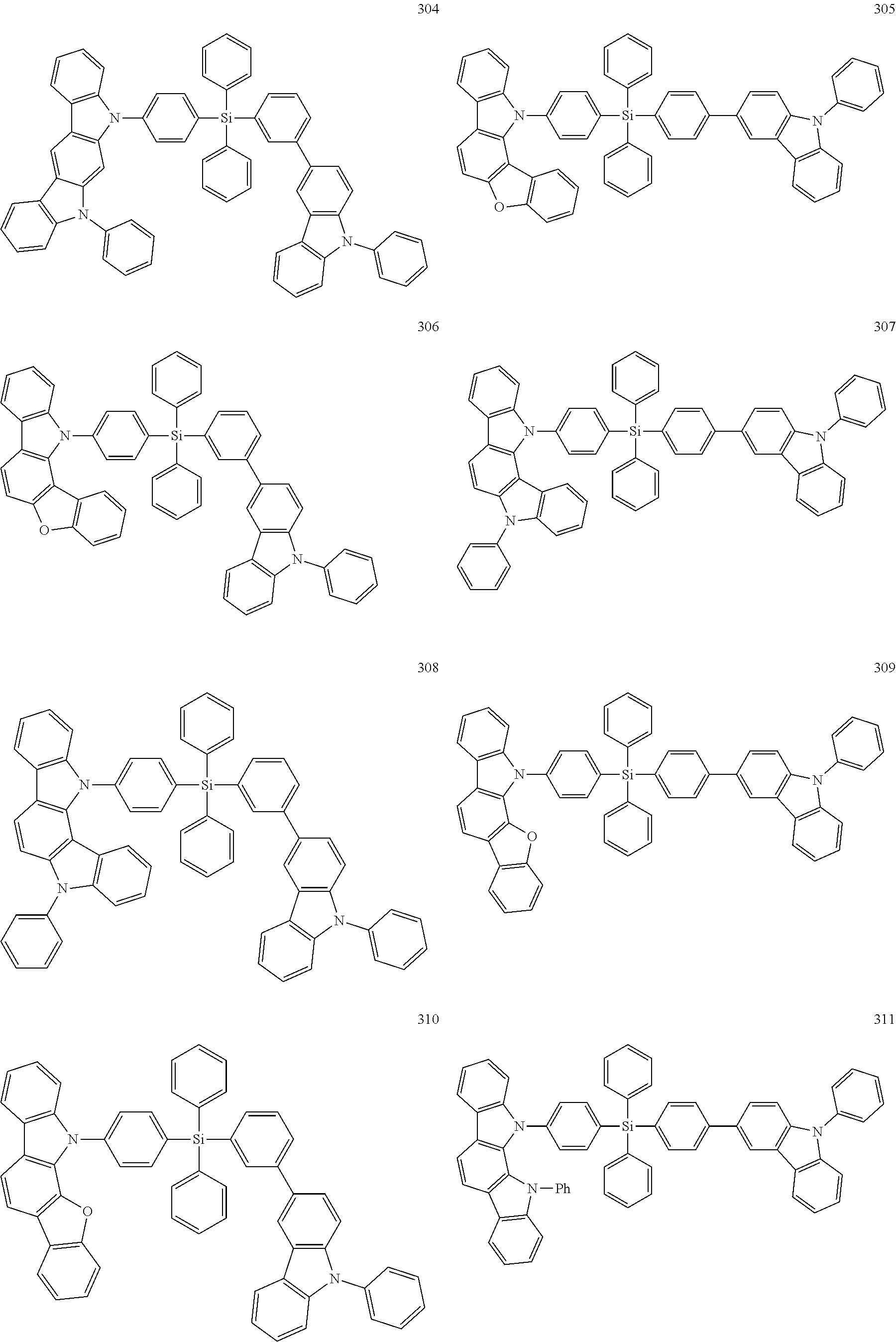

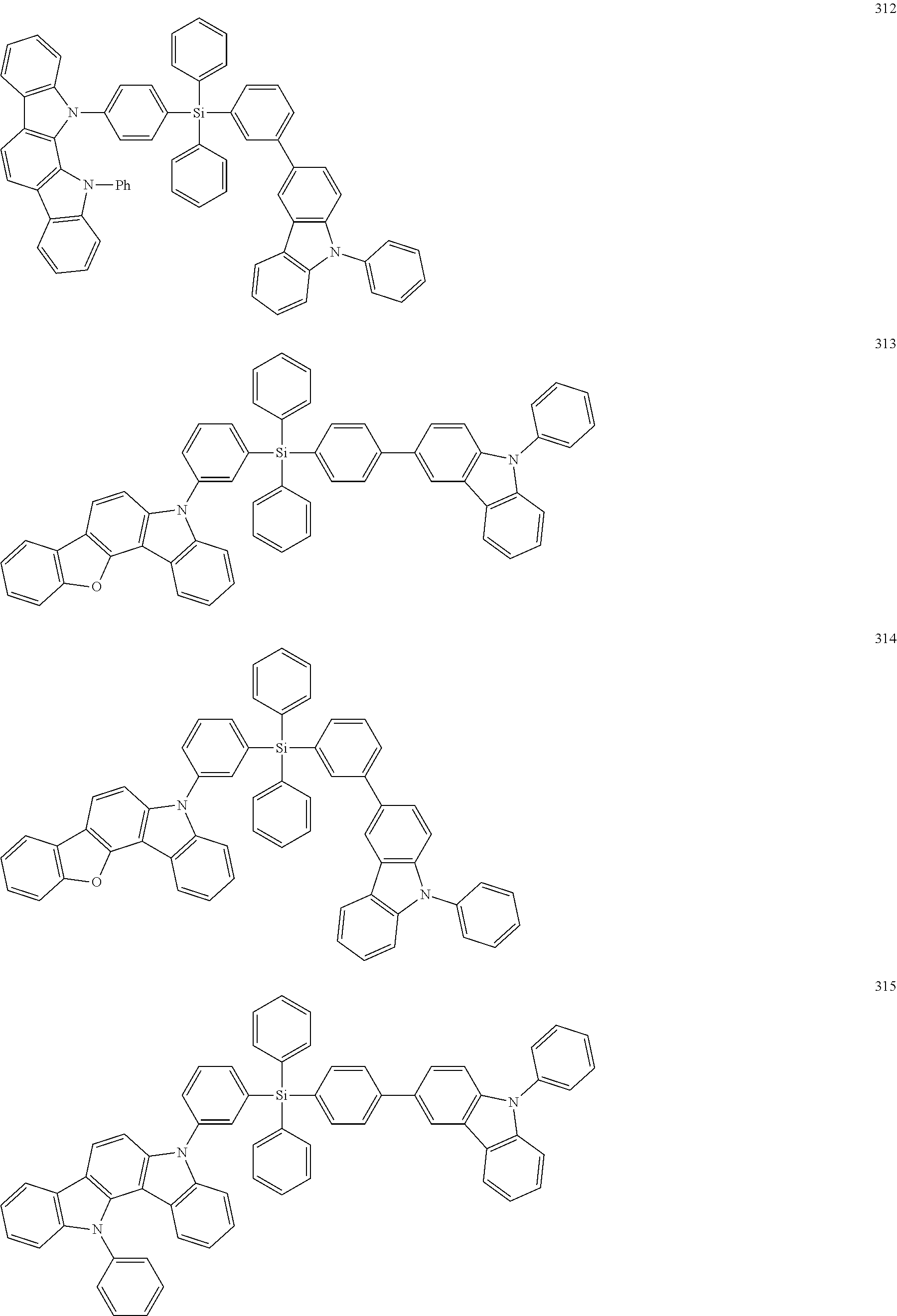

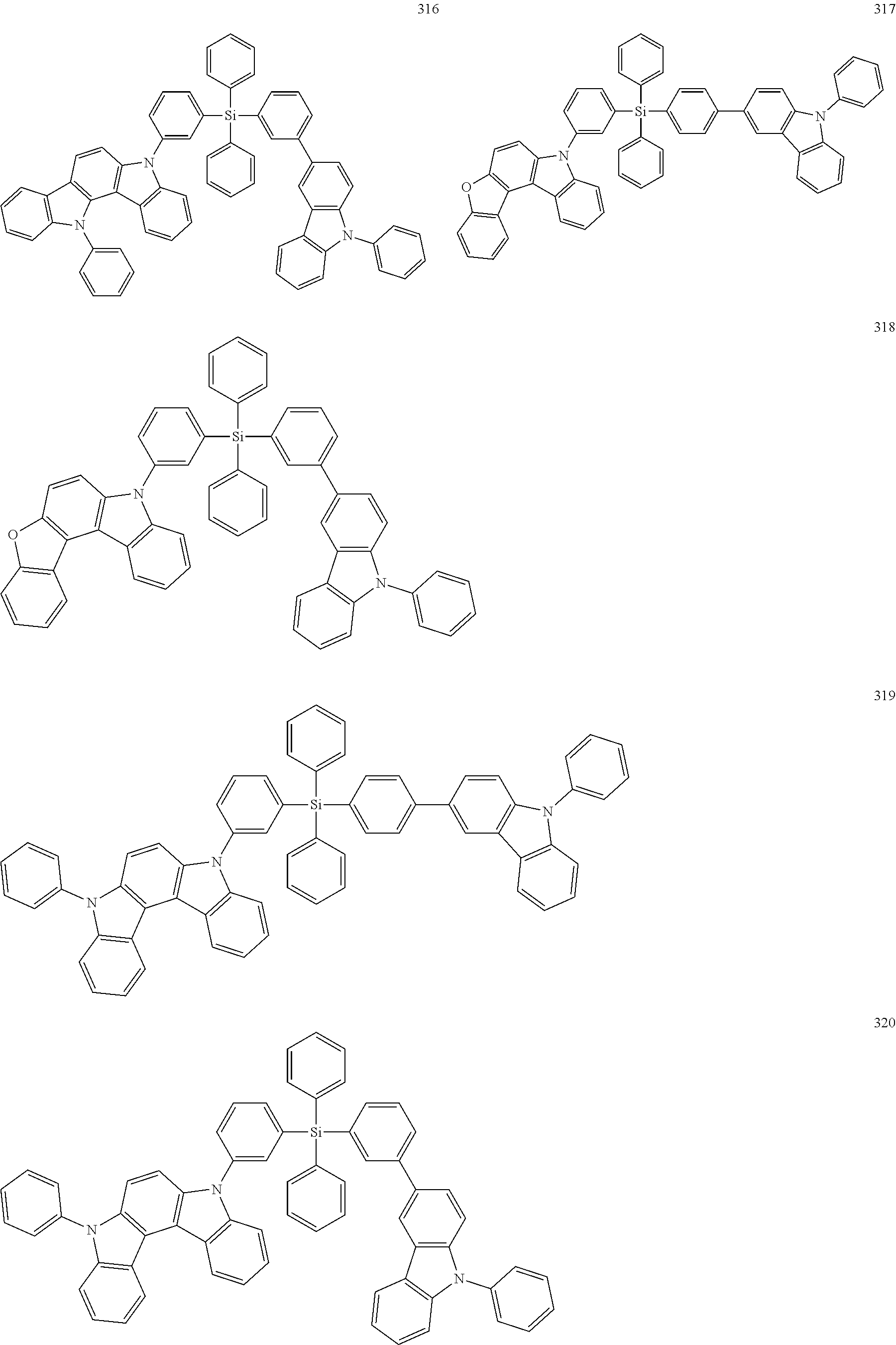

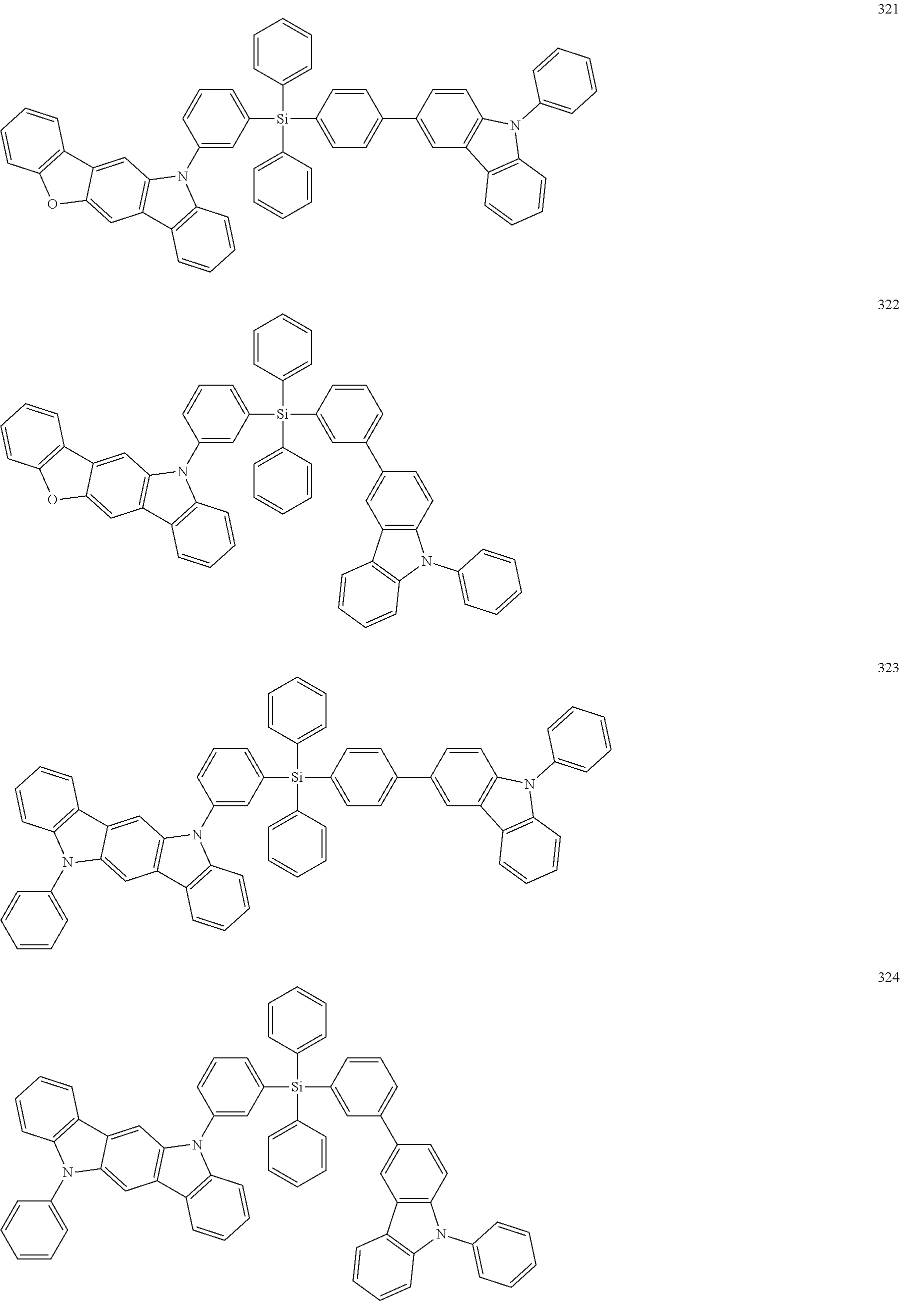 [SMILES: c1ccc(-n2c3ccccc3c3cc(-c4ccc([Si](c5ccccc5)(c5ccccc5)c5cccc(-n6c7ccccc7c7cc8oc9ccccc9c8cc76)c5)cc4)ccc32)cc1 ; c1ccc(-n2c3ccccc3c3cc(-c4cccc([Si](c5ccccc5)(c5ccccc5)c5cccc(-n6c7ccccc7c7cc8oc9ccccc9c8cc76)c5)c4)ccc32)cc1 ; c1ccc(-n2c3ccccc3c3cc(-c4ccc([Si](c5ccccc5)(c5ccccc5)c5cccc(-n6c7ccccc7c7cc8c(cc76)c6ccccc6n8-c6ccccc6)c5)cc4)ccc32)cc1 ; c1ccc(-n2c3ccccc3c3cc(-c4cccc([Si](c5ccccc5)(c5ccccc5)c5cccc(-n6c7ccccc7c7cc8c(cc76)c6ccccc6n8-c6ccccc6)c5)c4)ccc32)cc1]

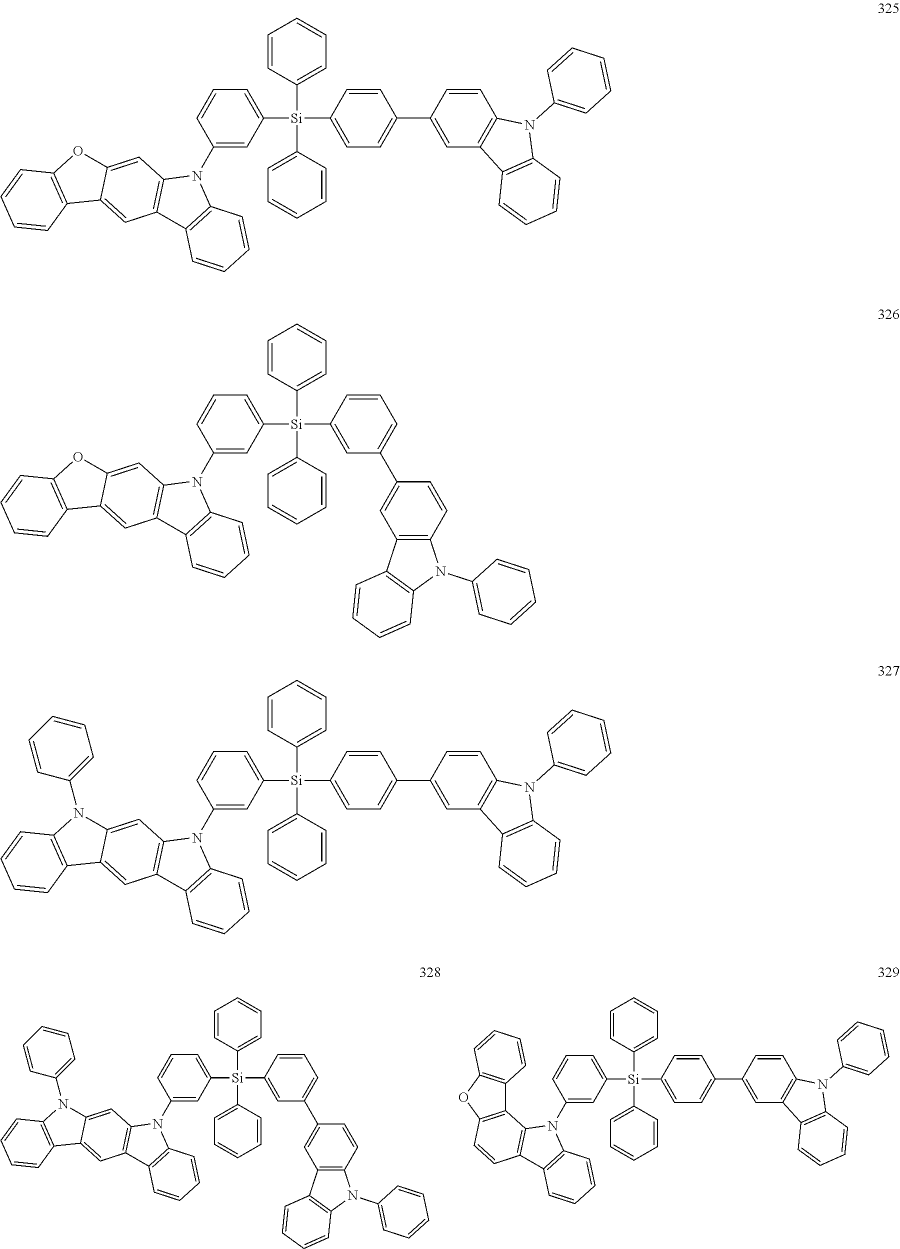

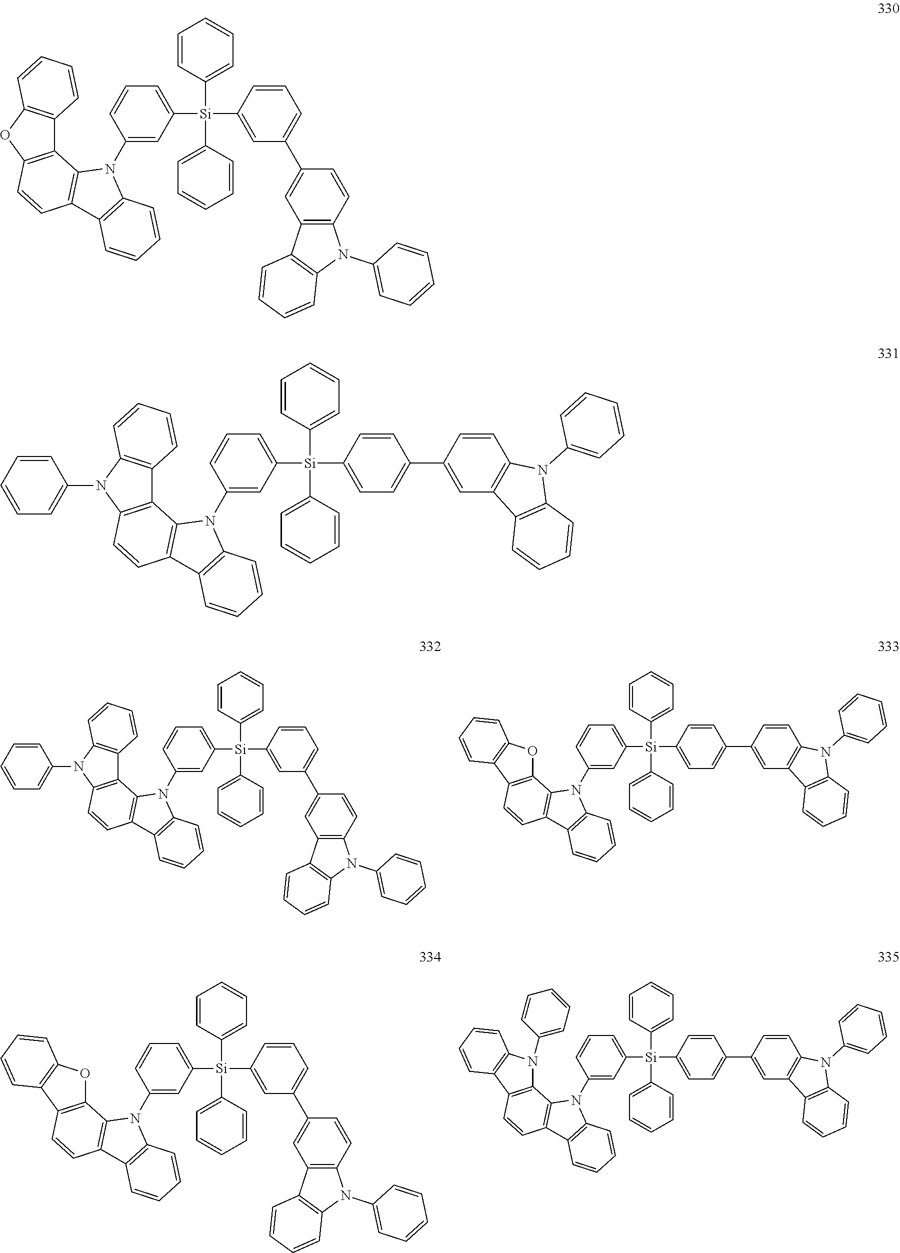

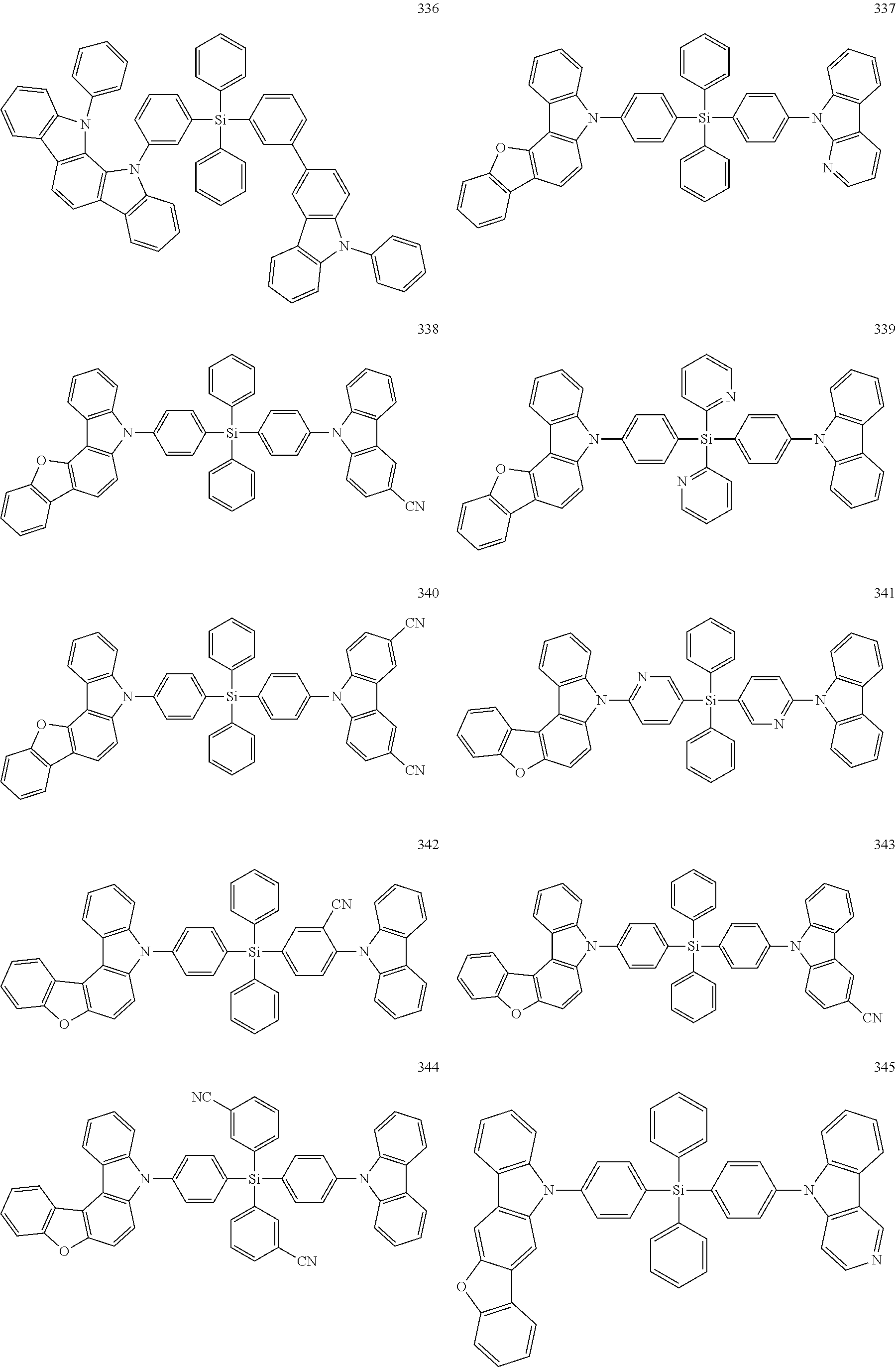

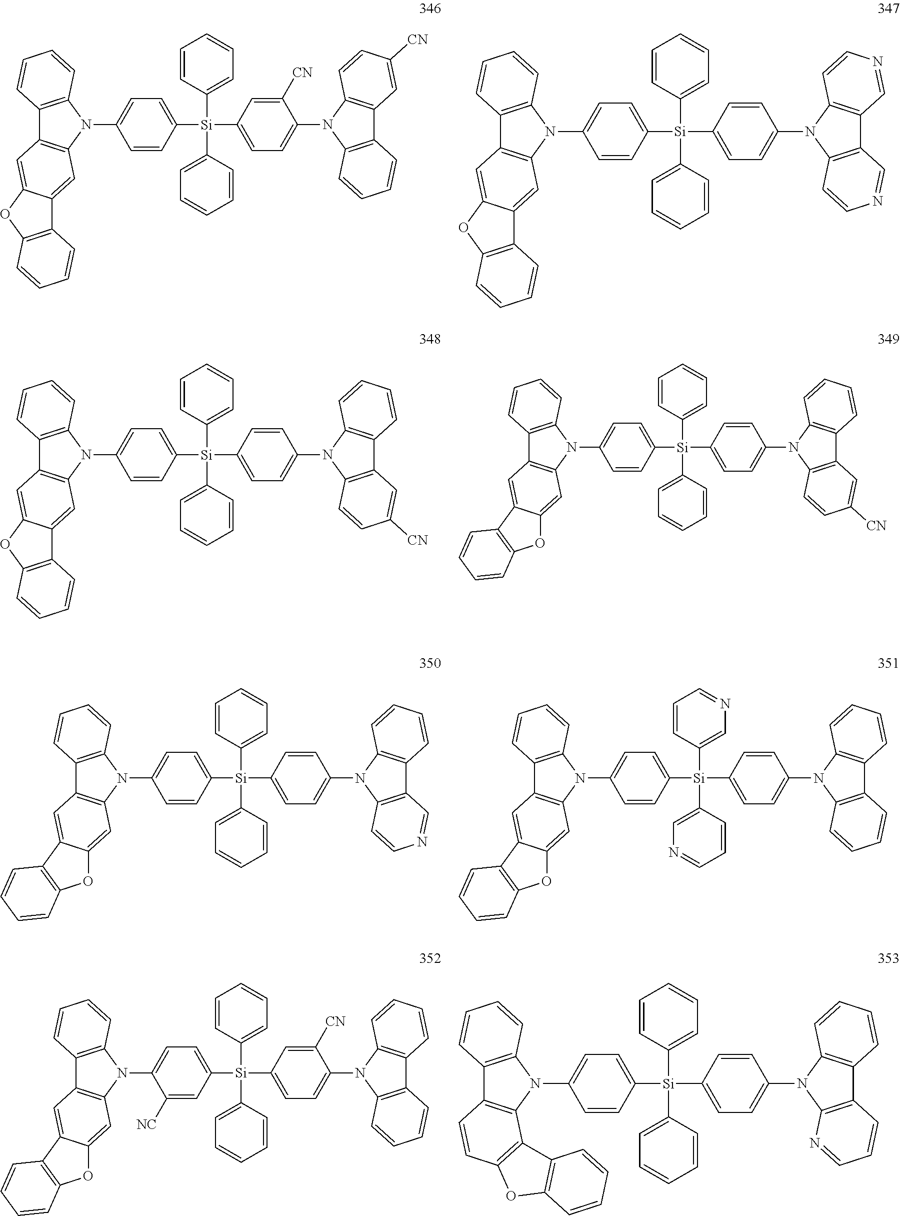

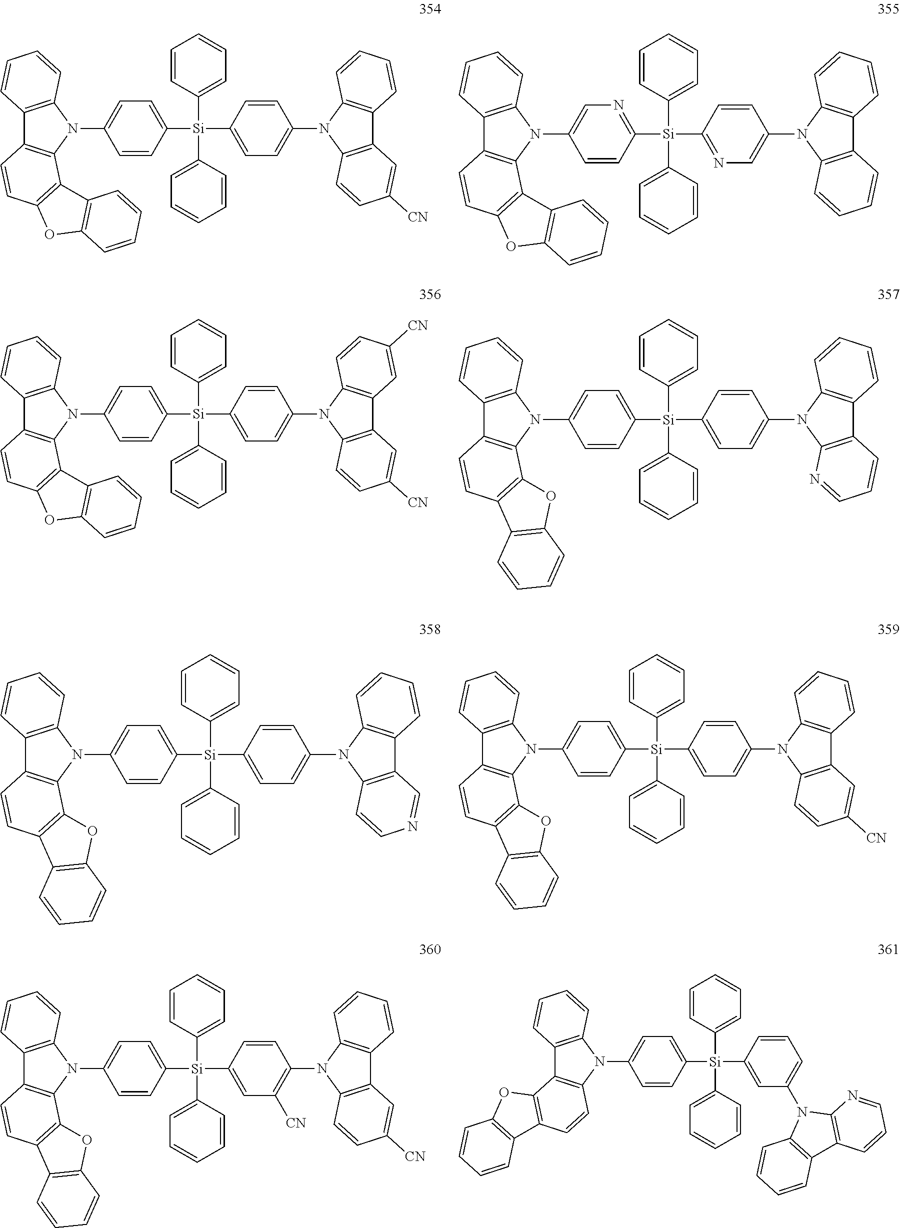

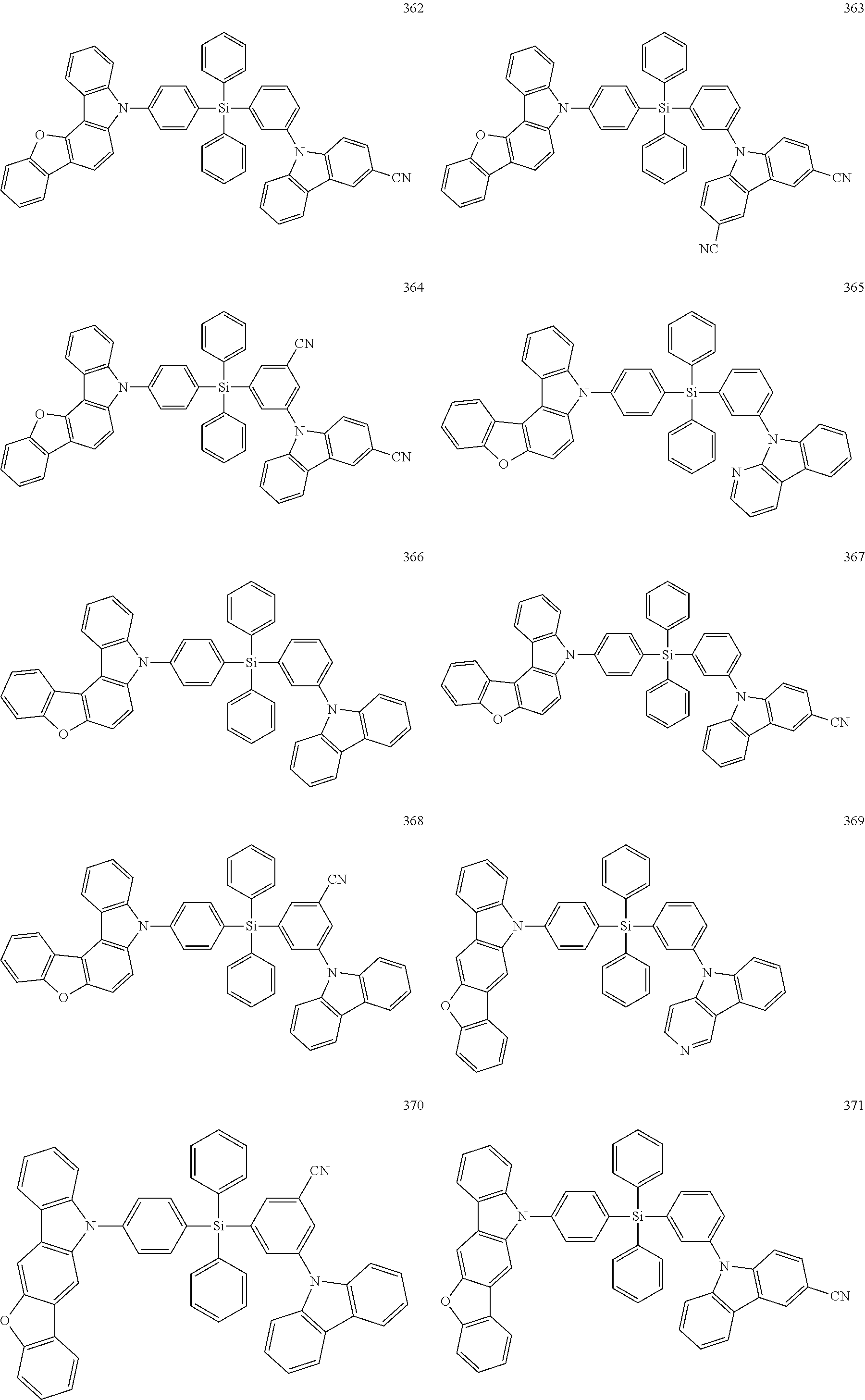

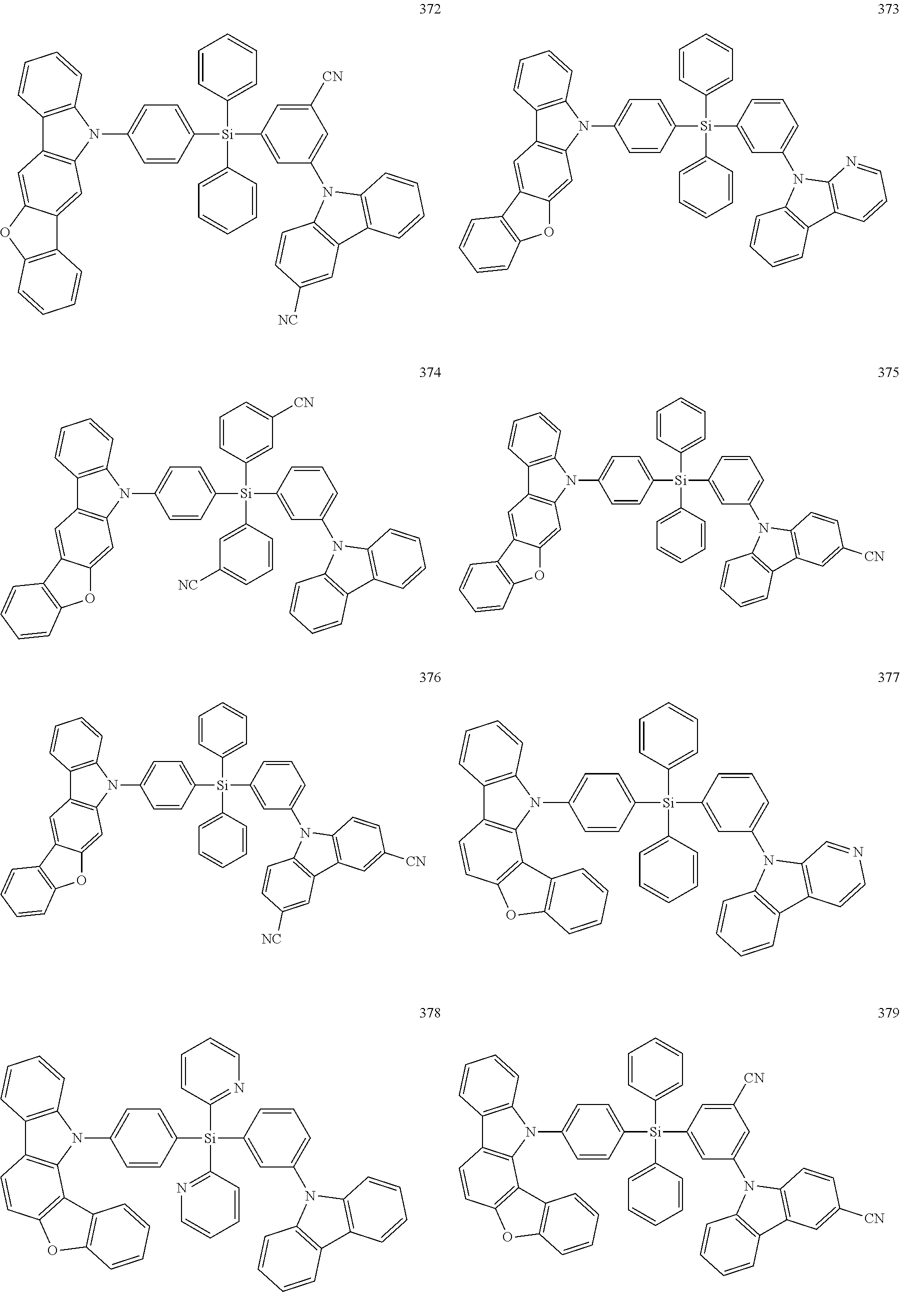

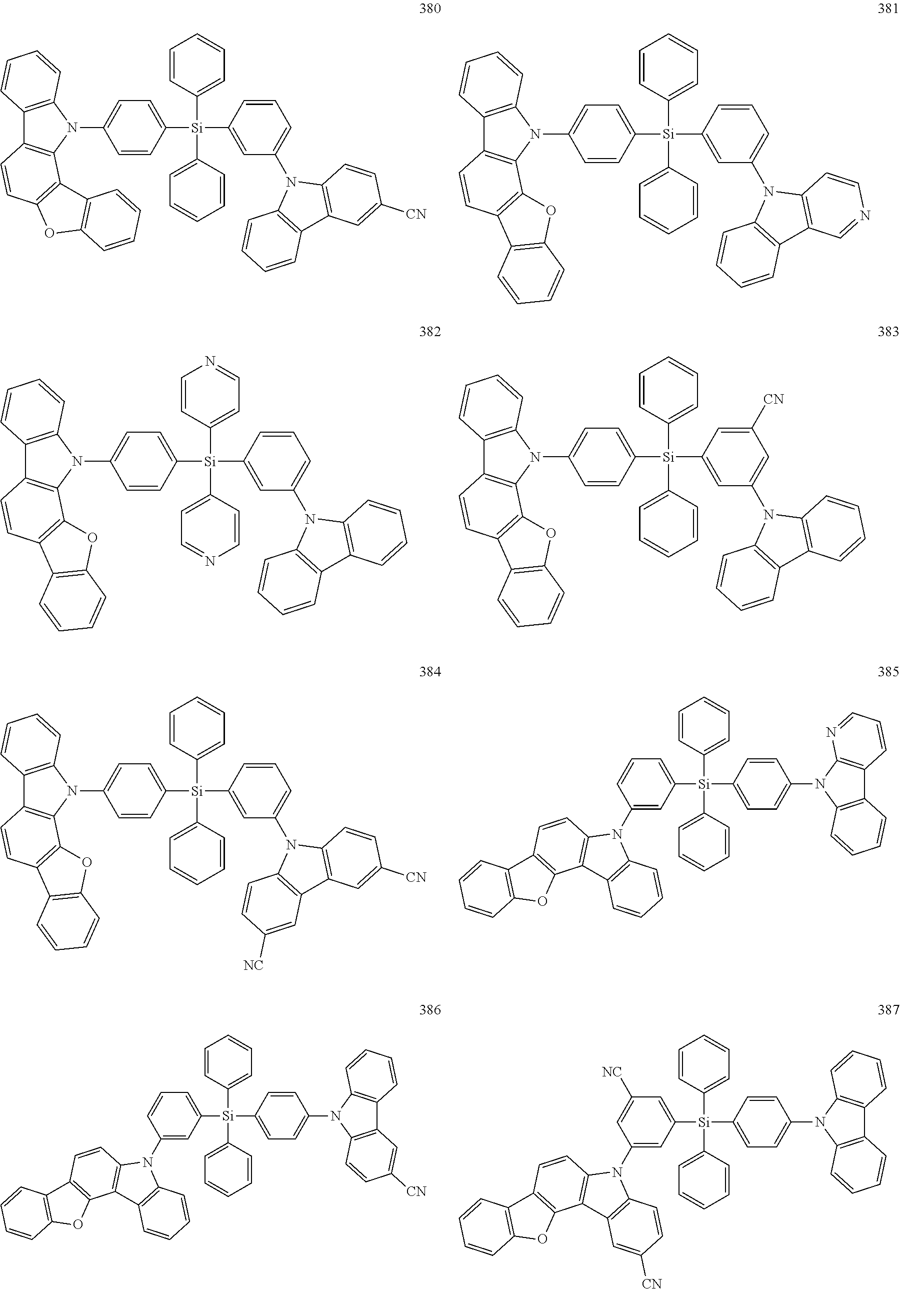

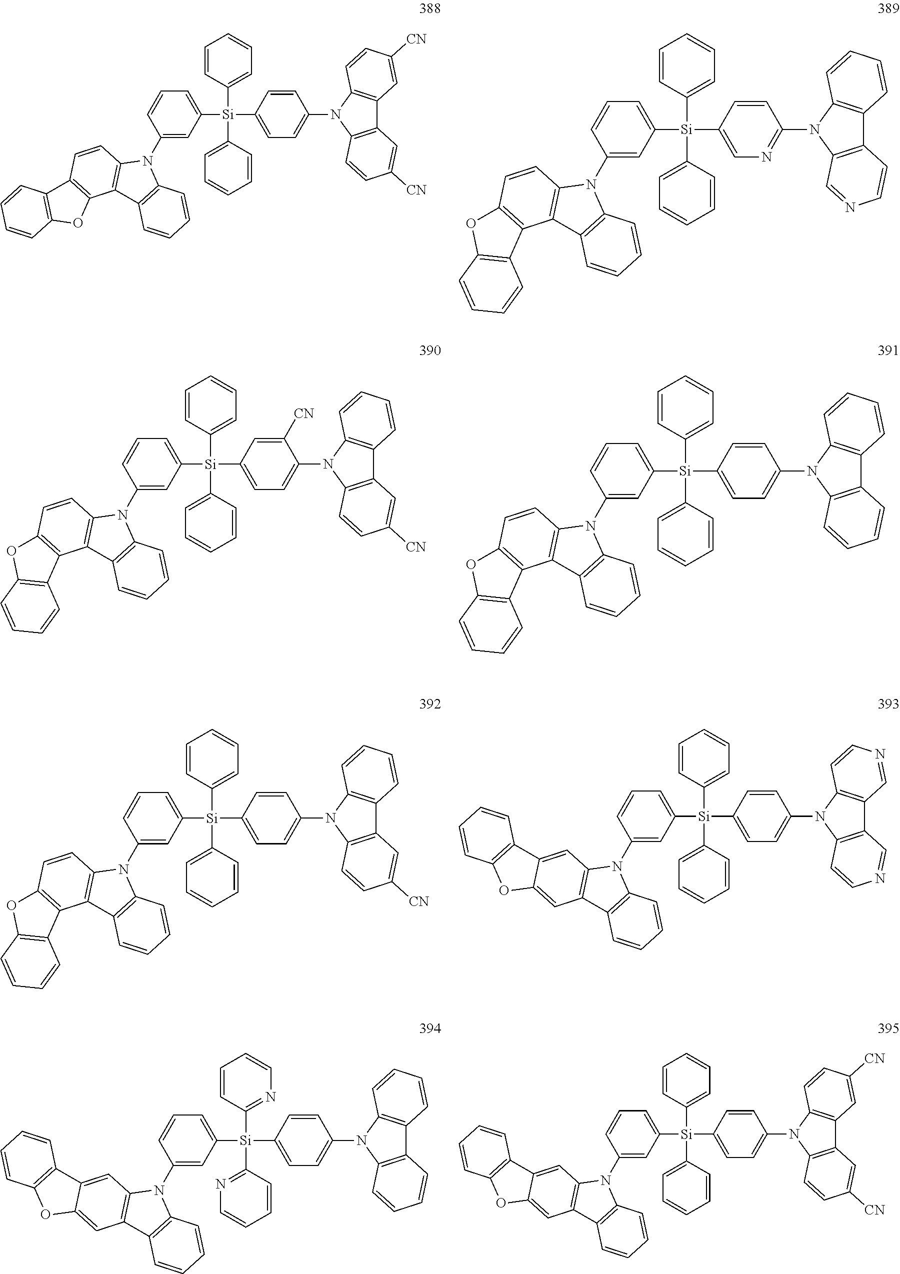

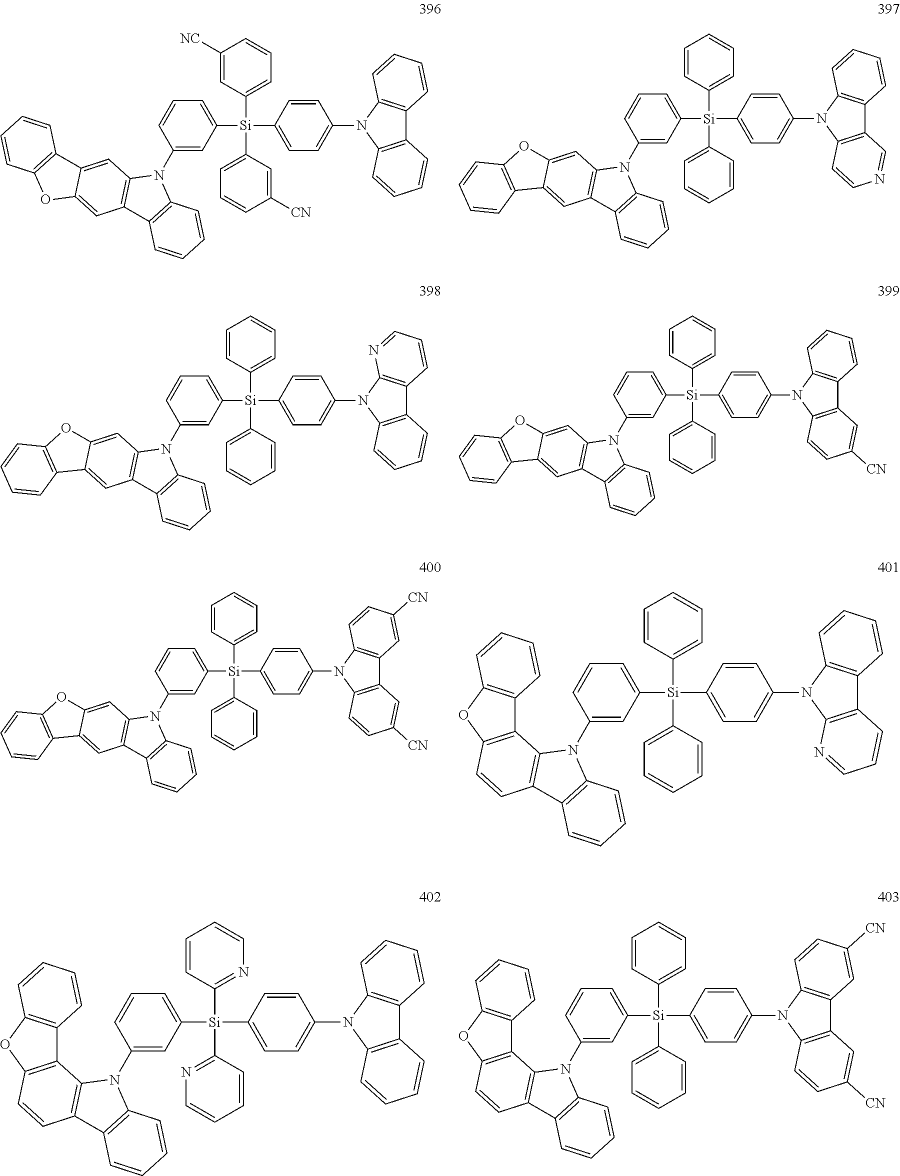

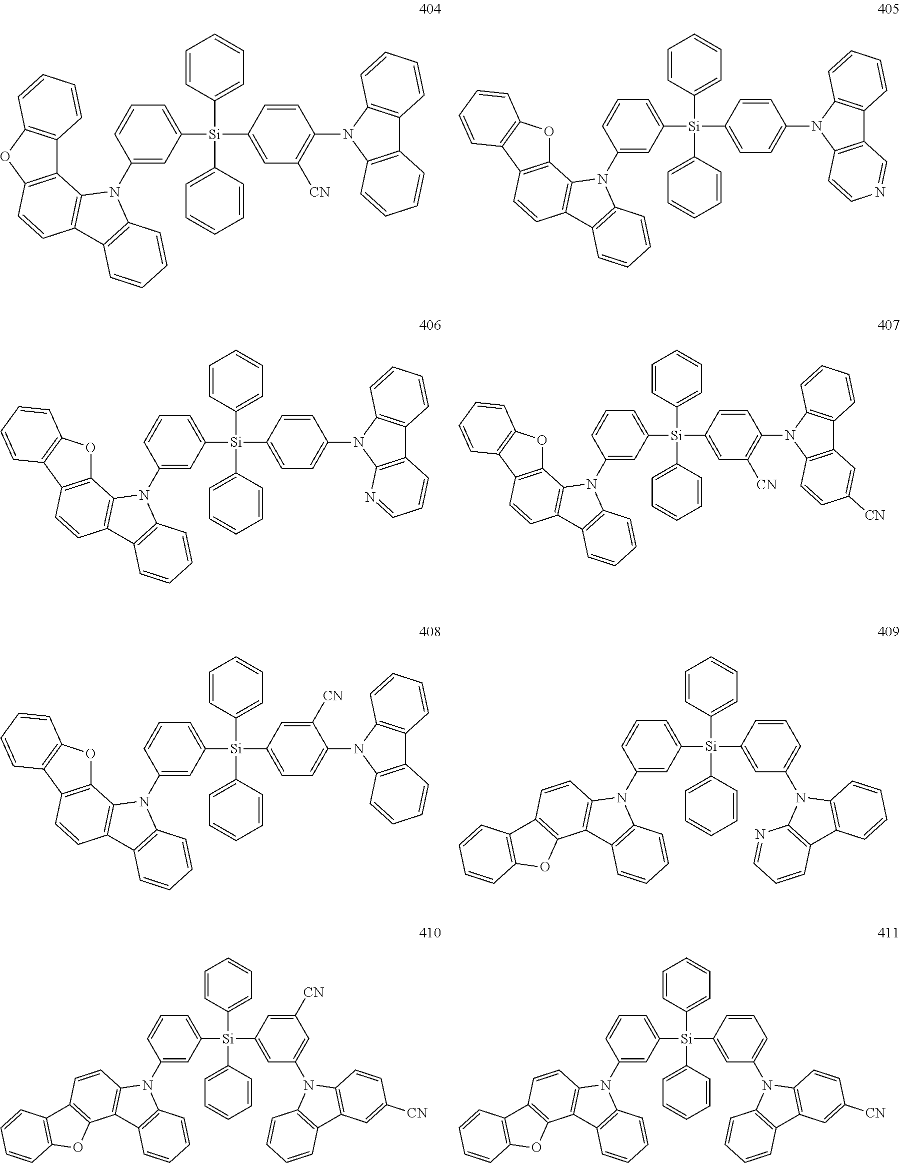

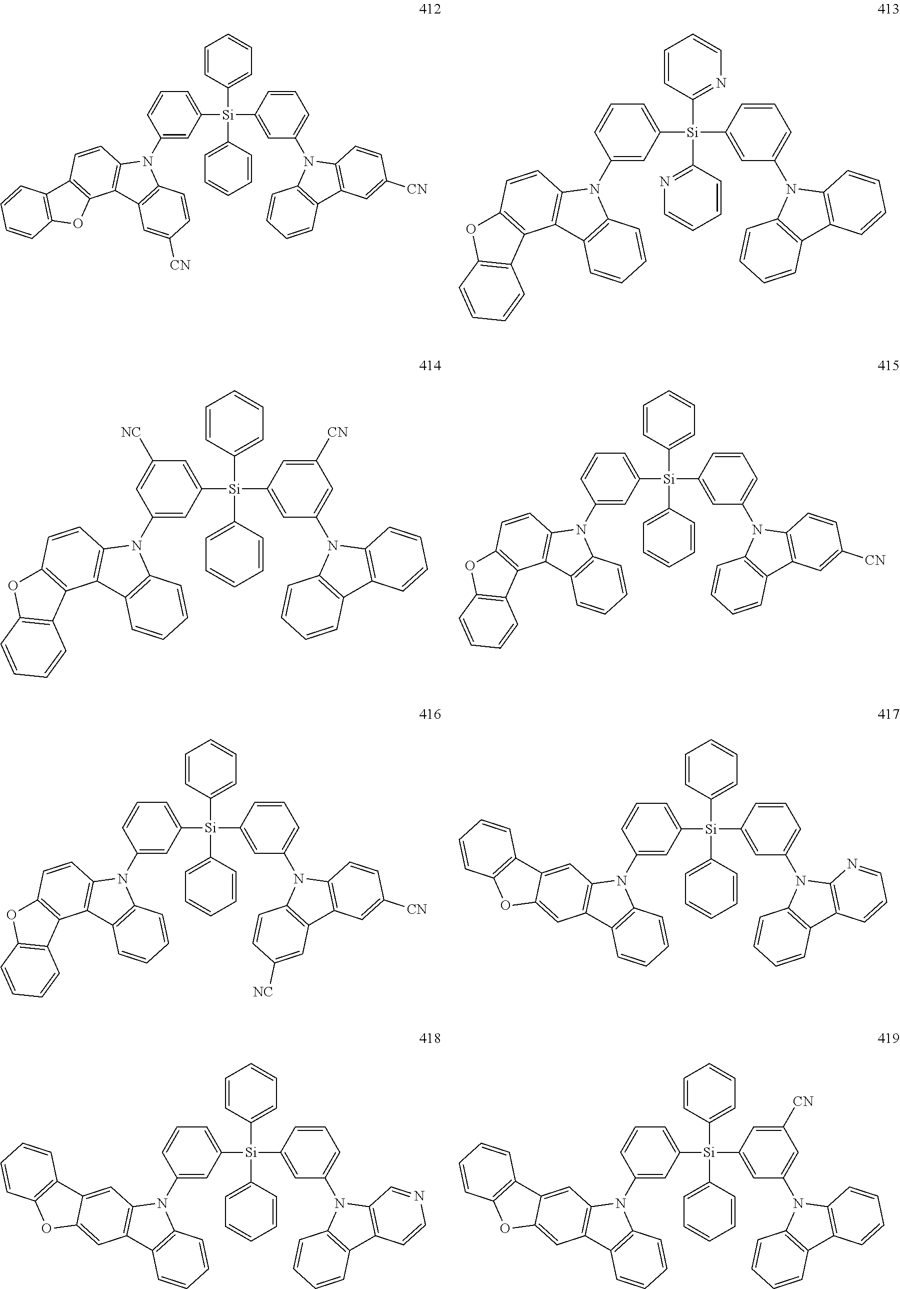

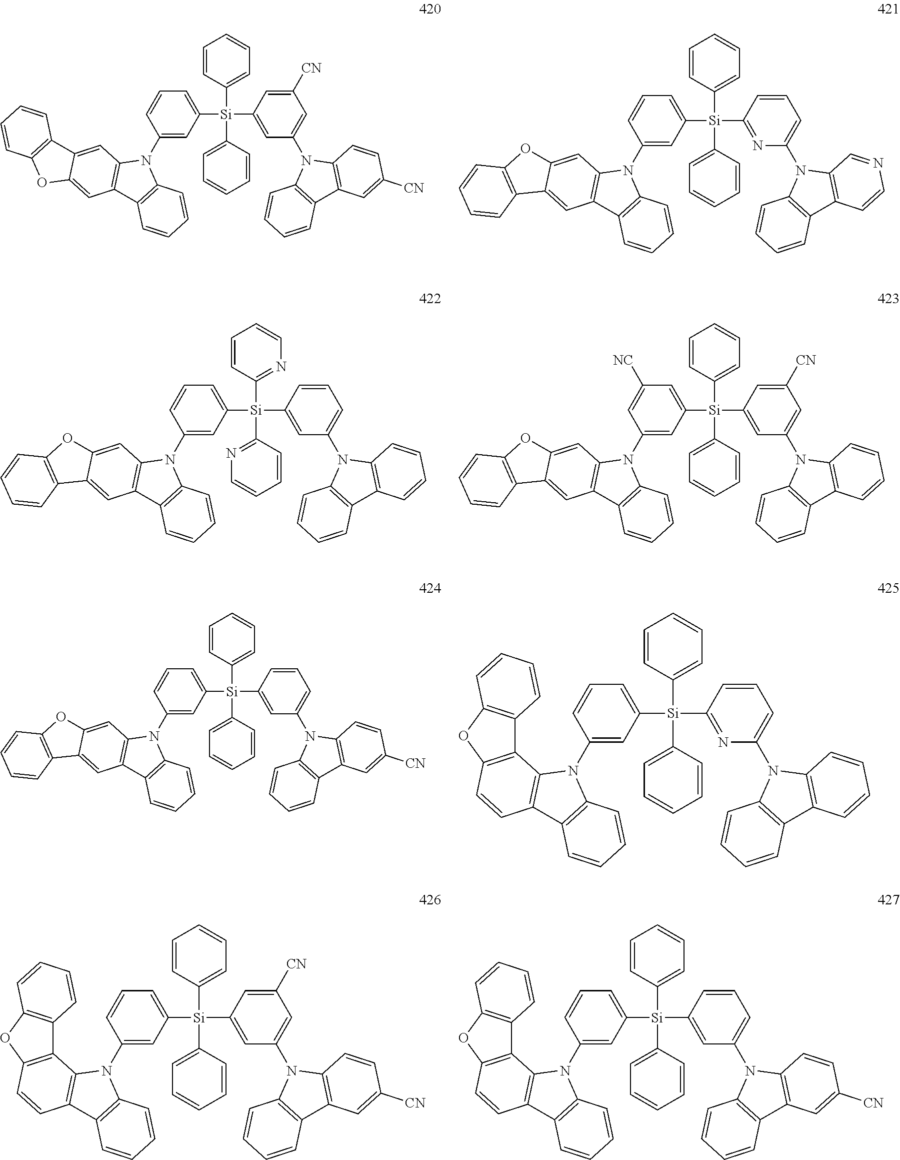

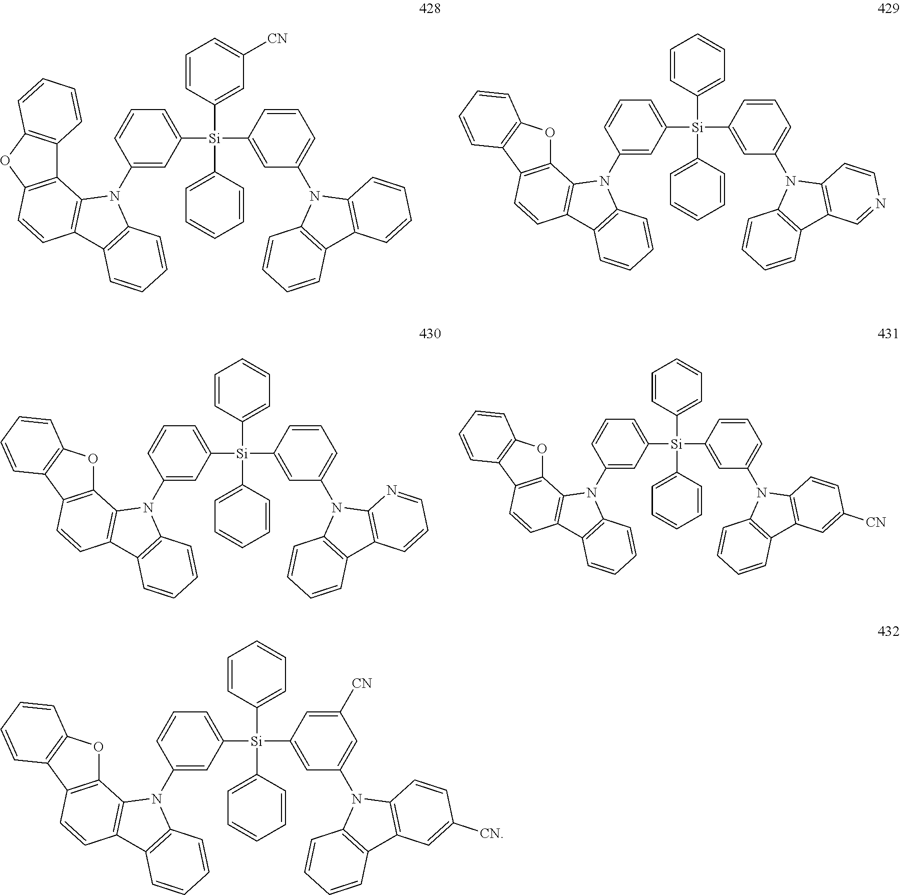

The term “Ph” as used herein is a phenyl group.

In one or more embodiments, an example of the hole-transporting host may be Compound H1, but embodiments are not limited thereto. In one or more embodiments, an example of the electron-transporting host may be Compound H2, but embodiments are not limited thereto:

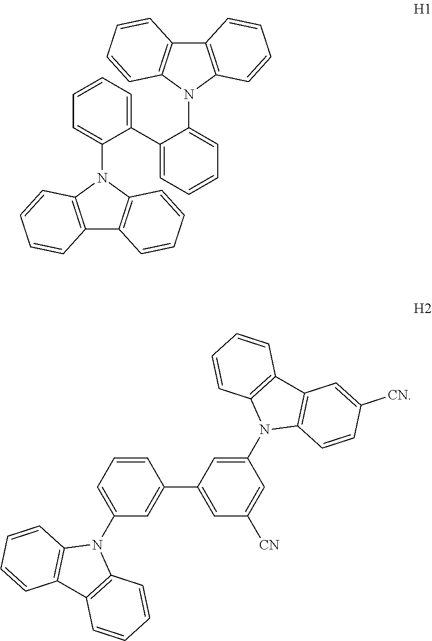 [SMILES: c1ccc(-n2c3ccccc3c3ccccc32)c(-c2ccccc2-n2c3ccccc3c3ccccc32)c1 ; N#Cc1cc(-c2cccc(-n3c4ccccc4c4ccccc43)c2)cc(-n2c3ccccc3c3cc(C#N)ccc32)c1]

### Dopant in Emission Layer 15

The dopant included in the emission layer 15 may include a phosphorescent dopant, a fluorescent dopant, or a combination thereof.

In one or more embodiments, the emission layer 15 may include a host, a fluorescent dopant, and a phosphorescent dopant, and the host may include at least one of the heterocyclic compounds represented by Formula 1. In this regard, the phosphorescent dopant may be a sensitizer compound that is used together with a fluorescent dopant to transfer excitons to the fluorescent dopant.

For example, the phosphorescent dopant may be a blue dopant.

### Phosphorescent Dopant

In one or more embodiments, the phosphorescent dopant may include a transition metal and a tetradentate ligand. In one or more embodiments, the phosphorescent dopant may include a transition metal and at least one of a monodentate ligand, a bidentate ligand, or a tridentate ligand.

In one or more embodiments, the phosphorescent dopant may include at least one organometallic compound represented by Formula 31:

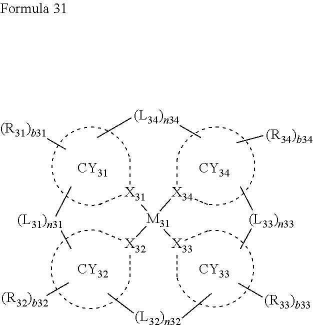

wherein, in Formula 31, M₃₁ may be a transition metal.

In one or more embodiments, M₃₁ may be iridium, platinum, palladium, gold, osmium, titanium, zirconium, hafnium, europium, terbium, thulium, or rhodium. In one or more embodiments, M₃₁ may be platinum, palladium, or gold.

In Formula 31, X₃₁ to X₃₄ may each independently be C or N, and two of a bond between X₃₁ and M₃₁, a bond between X₃₂ and M₃₁, a bond between X₃₃ and M₃₁, and a bond between X₃₄ and M₃₁ may be coordinate bonds, and the other two may be covalent bonds.

In one or more embodiments, a bond between X₃₁ and M₃₁ may be a coordinate bond.

In one or more embodiments, X₃₁ may be C, and a bond between X₃₁ and M₃₁ may be a coordinate bond. That is, X₃₁ in Formula 3 may be C in a carbene moiety.

In Formula 31, ring CY₃₁ to ring CY₃₄ may each independently be a C₅-C₃₀ carbocyclic group or a C₁-C₃₀ heterocyclic group.

In one or more embodiments, ring CY₃₁ to ring CY₃₄ may each independently be:
i) a first ring, ii) a second ring, iii) a condensed ring group in which two or more first rings are condensed with each other, iv) a condensed ring group in which two or more second rings are condensed with each other or v) a condensed ring group in which at least one first ring is condensed with at least one second ring,
wherein the first ring may be a cyclopentane group, a cyclopentadiene group, a furan group, a thiophene group, a pyrrole group, a silole group, an oxazole group, an isoxazole group, an oxadiazole group, an isoxadiazole group, an oxatriazole group, an isoxatriazole group, a thiazole group, an isothiazole group, a thiadiazole group, an isothiadiazole group, a thiatriazole group, an isothiatriazole group, a pyrazole group, an imidazole group, a triazole group, a tetrazole group, an azasilole group, a diazasilole group, or a triazasilole group, and
the second ring may be an adamantane group, a norbornane group, a norbornene group, a cyclohexane group, a cyclohexene group, a benzene group, a pyridine group, a pyrimidine group, a pyrazine group, a pyridazine group, a triazine group, an oxazine group, a thiazine group, a dihydropyrazine group, a dihydropyridine group, or a dihydroazasilole group.

In Formula 31, L₃₁ may be a single bond, a double bond, *—N(R₃₅ₐ)—*′, *—B(R₃₅ₐ)—*′ *—P(R₃₅ₐ)—*′, *—C(R₃₅ₐ)(R_{35b})—*′, *—Si(R₃₅ₐ)(R_{35b})—*′, *—Ge(R₃₅ₐ)(R_{35b})—*′, *—S—*′, *—Se—*′, *—O—*′, *—C(═O)—*′, *—S(═O)—*′, *—S(═O)₂—*′ *—C(R₃₅ₐ)═*′, *═C(R₃₅ₐ)—*′, *—C(R₃₅ₐ)═C(R_{35b})—*′, *—C(═S)—*′, *—C≡C—*′, a C₅-C₃₀ carbocyclic group unsubstituted or substituted with at least one R₁₀ₐ, or a C₁-C₃₀ heterocyclic group unsubstituted or substituted with at least one R₁₀ₐ, L₃₂ may be a single bond, a double bond, *—N(R₃₆ₐ)—*′, *—B(R₃₆ₐ)—*′, *—P(R₃₆ₐ)—*′, *—C(R₃₆ₐ)(R_{36b})—*′, *—Si(R₃₆ₐ)(R_{36b})—*′, *—Ge(R₃₆ₐ)(R_{36b})—*′, *—S—* , *—Se—*′, *—O—*′, *—C(═O)—*′, *—S(═O)—*′, *—S(═O)₂—*′ *—C(R₃₆ₐ)═*′, *═C(R₃₆ₐ)—*′, *—C(R₃₆ₐ)═C(R_{36b})—*′, *—C(═S)—*′, *—C≡C—*′, a C₅-C₃₀ carbocyclic group unsubstituted or substituted with at least one R₁₀ₐ, or a C₁-C₃₀ heterocyclic group unsubstituted or substituted with at least one R₁₀ₐ,
L₃₃ may be a single bond, a double bond, *—N(R₃₇ₐ)—*′, *—B(R₃₇ₐ)—*′, *—P(R₃₇ₐ)—*′, *—C(R₃₇ₐ)(R_{37b})—*′, *—Si(R₃₇ₐ)(R_{37b})—*′, *—Ge(R₃₇ₐ)(R_{37b})—*′, *—S—*′, *—Se—*′, *—O—*′, *—C(═O)—*′, *—S(═O)—*′, *—S(═O)₂—*′ *—C(R₃₇ₐ)═*′, *═C(R₃₇ₐ)—*′, *—C(R₃₇ₐ)═C(R_{37b})—*′, *—C(═S)—*′, *—C≡C—*′, a C₅-C₃₀ carbocyclic group unsubstituted or substituted with at least one R₁₀ₐ, or a C₁-C₃₀ heterocyclic group unsubstituted or substituted with at least one R₁₀ₐ, and
L₃₄ may be a single bond, a double bond, *—N(R₃₈ₐ)—*′, *—B(R₃₈ₐ)—*′, *—P(R₃₈ₐ)—*′, *—C(R₃₈ₐ)(R_{38b})—*′, *—Si(R₃₈ₐ)(R_{38b})—*′, *—Ge(R₃₈ₐ)(R_{38b})—*′, *—S—*′, *—Se—*′, *—O—*′, *—C(═O)—*′, *—S(═O)—*′, *—S(═O)₂—*′ *—C(R₃₈ₐ)═*′, *═C(R₃₈ₐ)—*′, *—C(R₃₈ₐ)═C(R_{38b})—*′, *—C(═S)—*′, *—C≡C—*′, a C₅-C₃₀ carbocyclic group unsubstituted or substituted with at least one R₁₀ₐ, or a C₁-C₃₀ heterocyclic group unsubstituted or substituted with at least one R₁₀ₐ.

In Formula 31, n31 to n34 may each independently be an integer from 0 to 5, wherein three or more of n31 to n34 may each independently be an integer from 1 to 5.

In Formula 31, when n31 is 0, L₃₁ is absent, when n32 is 0, L₃₂ is absent, when n33 is 0, L₃₃ is absent, and when n34 is 0, L₃₄ is absent.

In Formula 31, when n31 is 2 or greater, two or more of L₃₁ may be identical to or different from each other, when n32 is 2 or greater, two or more of L₃₂ may be identical to or different from each other, when n33 is 2 or greater, two or more of L₃₃ may be identical to or different from each other, and when n34 is 2 or more, two or greater of L₃₄ may be identical to or different from each other.

In Formula 31, R₃₁ to R₃₄, R₃₅ₐ, R_{35b}, R₃₆ₐ, R_{36b}, R₃₇ₐ, R_{37b}, R₃₈ₐ, and R_{38b} may each independently be hydrogen, deuterium, —F, —Cl, —Br, —I, —SF₅, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₁-C₆₀ alkylthio group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₇-C₆₀ alkyl aryl group, a substituted or unsubstituted C₇-C₆₀ aryl alkyl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted C₂-C₆₀ heteroaryl alkyl group, a substituted or unsubstituted C₂-C₆₀ heteroaryl alkyl group, a substituted or unsubstituted C₁-C₆₀ heteroaryloxy group, a substituted or unsubstituted C₁-C₆₀ heteroarylthio group, a substituted or unsubstituted monovalent non-aromatic condensed polycyclic group, a substituted or unsubstituted monovalent non-aromatic condensed heteropolycyclic group, —N(Q₁)(Q₂), —Si(Q₃)(Q₄)(Q₅), —Ge(Q₃)(Q₄)(Q₅), —B(Q₆)(Q₇), —P(═O)(Q₈)(Q₉), or —P(Q₈)(Q₉).

In one or more embodiments, R₃₁ to R₃₄, R₃₅ₐ, R_{35b}, R₃₆ₐ, R_{36b}, R₃₇ₐ, R_{37b}, R₃₈ₐ, and R_{38b} may each independently be:
hydrogen, deuterium, —F, —Cl, —Br, —I, —SF₅, —CD₃, —CD₂H, —CDH₂, —CF₃, —CF₂H, —CFH₂, a cyano group, a nitro group, an amino group, a C₁-C₂₀ alkyl group, a C₁-C₂₀ alkoxy group, or a C₁-C₂₀ alkylthio group;
a C₁-C₂₀ alkyl group, a C₁-C₂₀ alkoxy group, or a C₁-C₂₀ alkylthio group, each substituted with at least one of deuterium, —F, —Cl, —Br, —I, —SF₅, —CD₃, —CD₂H, —CDH₂, —CF₃, —CF₂H, —CFH₂, a cyano group, a nitro group, an amino group, a phenyl group, or a combination thereof; or
a cyclopentyl group, a cyclohexyl group, a cycloheptyl group, a cyclopentenyl group, a cyclohexenyl group, a cycloheptenyl group, a phenyl group, a biphenyl group, a naphthyl group, a fluorenyl group, a phenanthrenyl group, or an anthracenyl group, each unsubstituted or substituted with at least one of deuterium, —F, —Cl, —Br, —I, —SF₅, —CD₃, —CD₂H, —CDH₂, —CF₃, —CF₂H, —CFH₂, a cyano group, a nitro group, an amino group, a C₁-C₂₀ alkyl group, a C₁-C₂₀ alkoxy group, a C₁-C₂₀ alkylthio group, a cyclopentyl group, a cyclohexyl group, a cycloheptyl group, a cyclopentenyl group, a cyclohexenyl group, a cycloheptenyl group, a phenyl group, a biphenyl group, a naphthyl group, a fluorenyl group, a phenanthrenyl group, an anthracenyl group, or a combination thereof.

In one or more embodiments, the phosphorescent dopant may include at least one of the organometallic compounds represented by Formulae 31-1 or 31-2:

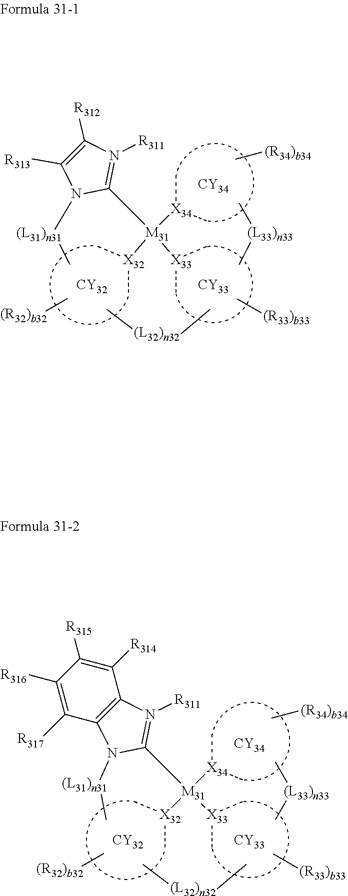

wherein, in Formula 31, b31 to b34 may each independently be an integer from 0 to 20.

In Formula 31, at least two of R₃₁ may optionally be bonded to each other to form a C₅-C₃₀ carbocyclic group unsubstituted or substituted with at least one R₁₀ₐ, or a C₁-C₃₀ heterocyclic group unsubstituted or substituted with at least one R₁₀ₐ,

At least two of R₃₂ may optionally be bonded to each other to form a C₅-C₃₀ carbocyclic group unsubstituted or substituted with at least one R₁₀ₐ, or a C₁-C₃₀ heterocyclic group unsubstituted or substituted with at least one R₁₀ₐ,
at least two of R₃₃ may optionally be bonded to each other to form a C₅-C₃₀ carbocyclic group unsubstituted or substituted with at least one R₁₀ₐ, or a C₁-C₃₀ heterocyclic group unsubstituted or substituted with at least one R₁₀ₐ,
at least two of R₃₄ may optionally be bonded to each other to form a C₅-C₃₀ carbocyclic group unsubstituted or substituted with at least one R₁₀ₐ, or a C₁-C₃₀ heterocyclic group unsubstituted or substituted with at least one R₁₀ₐ,
two or more of R₃₁ to R₃₄, R₃₅ₐ, R_{35b}, R₃₆ₐ, R_{36b}, R₃₇ₐ, R_{37b}, R₃₈ₐ, and R_{38b} may optionally be bonded to each other to form a C₅-C₃₀ carbocyclic group unsubstituted or substituted with at least one R₁₀ₐ, or a C₁-C₃₀ heterocyclic group unsubstituted or substituted with at least one R₁₀ₐ, and
R₁₀ₐ may be as described herein in connection with R₃₁.

In Formulae 31-1 and 31-2,
M₃₁, ring CY₃₂, ring CY₃₃, ring CY₃₄, X₃₂, X₃₃, X₃₄, L₃₁, L₃₂, L₃₃, n31, n32, n33, R₃₂, R₃₃, R₃₄, a32, a33, and a34 may each be as described herein, and
R₃₁₁ to R₃₁₇ may each independently be as described in connection with R₃₁.

In one or more embodiments, in Formulae 31-1 and 31-2, R₃₁₁ to R₃₁₇ may each independently be:
hydrogen, deuterium, —F, —Cl, —Br, —I, —SF₅, —CD₃, —CD₂H, —CDH₂, —CF₃, —CF₂H, —CFH₂, —SF₅, a hydroxyl group, a cyano group, a nitro group, an amino group, an amidino group, a hydrazine group, a hydrazone group, a carboxylic acid group or a salt thereof, a sulfonic acid group or a salt thereof, or a phosphoric acid group or a salt thereof;
a C₁-C₂₀ alkyl group, a C₁-C₂₀ alkoxy group, or a C₁-C₂₀ alkylthio group, each unsubstituted or substituted with at least one of deuterium, —F, —Cl, —Br, —I, —SF₅, —CD₃, —CD₂H, —CDH₂, —CF₃, —CF₂H, —CFH₂, —SF₅, a hydroxyl group, a cyano group, a nitro group, an amino group, an amidino group, a hydrazine group, a hydrazone group, or a combination thereof;
a C₃-C₁₀ cycloalkyl group, a C₁-C₁₀ heterocycloalkyl group, a C₆-C₆₀ aryl group, a C₇-C₆₀ alkyl aryl group, a C₆-C₆₀ aryloxy group, a C₆-C₆₀ arylthio group, a C₁-C₆₀ heteroaryl group, a substituted or unsubstituted C₂-C₆₀ alkyl heteroaryl group, a C₁-C₆₀ heteroaryloxy group, a C₁-C₆₀ heteroarylthio group, a monovalent non-aromatic condensed polycyclic group, a monovalent non-aromatic condensed heteropolycyclic group, each unsubstituted or substituted with at least one of deuterium, —F, —Cl, —Br, —I, —SF₅, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a C₁-C₂₀ alkyl group, a C₁-C₂₀ alkoxy group, a C₁-C₂₀ alkylthio group, or a combination thereof;
—N(Q₁)(Q₂), —Si(Q₃)(Q₄)(Q₅), —Ge(Q₃)(Q₄)(Q₅), —B(Q₆)(Q₇), —P(═O)(Q₈)(Q₉), or —P(Q₈)(Q₉).

For example, in Formulae 31-1 and 31-2, at least one of R₃₁₁ to R₃₁₇ may include a C₁-C₂₀ alkyl group, a C₆-C₆₀ aryl group, a C₇-C₆₀ alkyl aryl group, or a C₇-C₆₀ aryl alkyl group, each unsubstituted or substituted with at least one of a methyl group, an ethyl group, an n-propyl group, an isopropyl group, an n-butyl group, an isobutyl group, a sec-butyl group, a tert-butyl group, a phenyl group, a cumyl group, or a combination thereof.

In one or more embodiments, the phosphorescent dopant may include an organometallic compound represented by Formula 51:

M₅₁(L₅₁)ₙ₅₁(L₅₂)ₙ₅₂ Formula 51

wherein M₅₁ in Formula 51 may be a transition metal.

In one or more embodiments, M₅₁ may be a first-row transition metal of the Periodic Table of Elements, a second-row transition metal of the Periodic Table of Elements, or a third-row transition metal of the Periodic Table of Elements.

In one or more embodiments, M₅₁ may be iridium, platinum, palladium, gold, osmium, titanium, zirconium, hafnium, europium, terbium, thulium, or rhodium.

In one or more embodiments, M₅₁ may be iridium, platinum, osmium, or rhodium.

In Formula 51, L₅₁ may be a ligand represented by Formula 51 Å, and L₅₂ may be a ligand represented by Formula 51B:

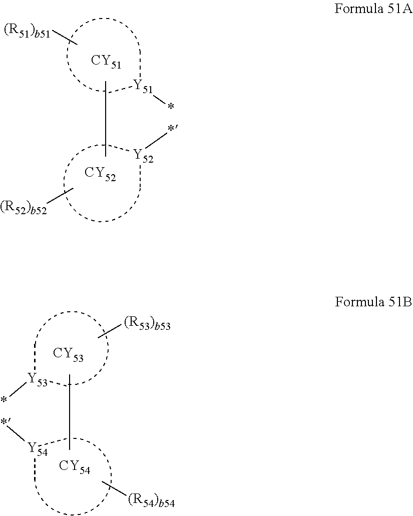

wherein Formulae 51A and 51B are each as described herein.

In Formula 51, n51 may be 1, 2, or 3, wherein, when n51 is 2 or greater, two or more of L₅₁ identical to or different from each other.

In Formula 52, n52 may be 0, 1, or 2, wherein, when n52 is 2 or greater, two or more of L₅₂ identical to or different from each other.

In Formula 51, a sum of n51 and n52 may be 2 or 3. For example, the sum of n51 and n52 may be 3.

In one or more embodiments, in Formula 51, i) M₅₁ may be iridium, and the sum of n51+n52 may be 3; or ii) M₅₁ may be platinum, and the sum of n51+n52 may be 2.

In one or more embodiments, in Formula 51, M₅₁ may be iridium, and regarding n51 and n52, i) n51 may be 1, and n52 may be 2; or ii) n51 may be 2, and n52 may be 1.

In Formula 51, L₅₁ and L₅₂ may be different from each other.

In Formulae 51A and 51B, Y₅₁ to Y₅₄ may each independently be C or N. For example, Y₅₁ and Y₅₃ may each be N, and Y₅₂ and Y₅₄ may each be C.

In Formulae 51A and 51B, ring CY₅₁ to ring CY₅₄ may each independently be a C₅-C₃₀ carbocyclic group or a C₁-C₃₀ heterocyclic group.

For example, ring CY₅₁ to ring CY₅₄ may each independently be i) a third ring, ii) a fourth ring, iii) a condensed ring group in which two or more third rings are condensed with each other, iv) a condensed ring group in which two or more fourth rings are condensed with each other, or v) a condensed ring group in which at least one third ring is condensed with at least one fourth ring,
wherein the third ring may be a cyclopentane group, a cyclopentene group, a furan group, a thiophene group, a pyrrole group, a silole group, a borole group, a phosphole group, a germole group, a selenophene group, an oxazole group, an oxadiazole group, an oxatriazole group, a thiazole group, a thiadiazole group, a thiatriazole group, a pyrazole group, an imidazole group, a triazole group, a tetrazole group, or an azasilole group, and
the fourth ring may be an adamantane group, a norbornane group, a norbornene group, a cyclohexane group, a cyclohexene group, a benzene group, a pyridine group, a pyrimidine group, a pyrazine group, a pyridazine group, or a triazine group.

In one or more embodiments, in Formulae 51A and 5B, ring CY₁ to ring CY₄ may each independently be a cyclopentane group, a cyclohexane group, a cyclohexene group, a benzene group, a naphthalene group, an anthracene group, a phenanthrene group, a triphenylene group, a pyrene group, a chrysene group, a 1,2,3,4-tetrahydronaphthalene group, a cyclopentadiene group, a pyrrole group, a furan group, a thiophene group, a silole group, a borole group, a phosphole group, a germole group, a selenophene group, an indene group, an indole group, a benzofuran group, a benzothiophene group, a benzosilole group, a benzoborole group, a benzophosphole group, a benzogermole group, a benzoselenophene group, a fluorene group, a carbazole group, a dibenzofuran group, a dibenzothiophene group, a dibenzosilole group, a dibenzoborole group, a dibenzophosphole group, a dibenzogermole group, a dibenzoselenophene group, a benzofluorene group, a benzocarbazole group, a naphthobenzofuran group, a naphthobenzothiophene group, a naphthobenzosilole group, a naphthobenzoborole group, a naphthobenzophosphole group, a naphthobenzogermole group, a naphthobenzoselenophene group, a dibenzofluorene group, a dibenzocarbazole group, a dinaphthofuran group, a dinaphthothiophene group, a dinaphthosilole group, a dinaphthoborole group, a dinaphthophosphole group, a dinaphthogermole group, a dinaphthoselenophene group, an indenophenanthrene group, an indolophenanthrene group, a phenanthrobenzofuran group, a phenanthrobenzothiophene group, a phenanthrobenzosilole group, a phenanthrobenzoborole group, a phenanthrobenzophosphole group, a phenanthrobenzogermole group, a phenanthrobenzoselenophene group, a dibenzothiophene 5-oxide group, a 9H-fluorene-9-one group, a dibenzothiophene group, a 5,5-dioxide group, an azaindene group, an azaindole group, an azabenzofuran group, an azabenzothiophene group, an azabenzosilole group, an azabenzoborole group, an azabenzophosphole group, an azabenzogermole group, an azabenzoselenophene group, an azafluorene group, an azacarbazole group, an azadibenzofuran group, an azadibenzothiophene group, an azadibenzosilole group, an azadibenzoborole group, an azadibenzophosphole group, an azadibenzogermole group, an azadibenzoselenophene group, an azabenzofluorene group, an azabenzocarbazole group, an azanaphthobenzofuran group, an azanaphthobenzothiophene group, an azanaphthobenzosilole group, an azanaphthobenzoborole group, an azanaphthobenzophosphole group, an azanaphthobenzogermole group, an azanaphthobenzoselenophene group, an azadibenzofluorene group, an azadibenzocarbazole group, an azadinaphthofuran group, an azadinaphthothiophene group, an azadinaphthosilole group, an azadinaphthoborole group, an azadinaphthophosphole group, an azadinaphthogermole group, an azadinaphthoselenophene group, an azaindenophenanthrene group, an azaindolophenanthrene group, an azaphenanthrobenzofuran group, an azaphenanthrobenzothiophene group, an azaphenanthrobenzosilole group, an azaphenanthrobenzoborole group, an azaphenanthrobenzophosphole group, an azaphenanthrobenzogermole group, an azaphenanthrobenzoselenophene group, an azadibenzothiophene 5-oxide group, an aza9H-fluorene-9-one group, an azadibenzothiophene 5,5-dioxide group, a pyridine group, a pyrimidine group, a pyrazine group, a pyridazine group, a triazine group, a quinoline group, an isoquinoline group, a quinoxaline group, a quinazoline group, a benzoquinoline group, a benzoisoquinoline group, a benzoquinoxaline group, a benzoquinazoline group, a phenanthroline group, a phenanthridine group, a pyrrole group, a pyrazole group, an imidazole group, a triazole group, an oxazole group, an isoxazole group, a thiazole group, an isothiazole group, an oxadiazole group, a thiadiazole group, an azasilole group, an azaborole group, an azaphosphole group, an azagermole group, an azaselenophene group, a benzopyrrole group, a benzopyrazole group, a benzimidazole group, a benzoxazole group, a benzisoxazole group, a benzothiazole group, a benzisothiazole group, a benzioxadiazole group, a benzothiadiazole group, a pyridinopyrrole group, a pyridinopyrazole group, a pyridinoimidazole group, a pyridinooxazole group, a pyridinoisoxazole group, a pyridinothiazole group, a pyridinoisothiazole group, a pyridinooxadiazole group, a pyridinothiadiazole group, a pyrimidinopyrrole group, a pyrimidinopyrazole group, a pyrimidinoimidazole group, a pyrimidinooxazole group, a pyrimidinoisoxazole group, a pyrimidinothiazole group, a pyrimidinoisothiazole group, a pyrimidinooxadiazole group, a pyrimidinothiadiazole group, a 5,6,7,8-tetrahydroisoquinoline group, a 5,6,7,8-tetrahydroquinoline group, an adamantane group, a norbornane group, a norbornene group, a benzene group condensed with a cyclohexane group, a benzene group condensed with a norbornane group, a pyridine group condensed with a cyclohexane group, or a pyridine group condensed with a norbornane group.

In one or more embodiments, ring CY₅₁ and ring CY₅₃ may be different from each other.

In one or more embodiments, ring CY₅₂ and ring CY₅₄ may be different from each other.

In one or more embodiments, ring CY₅₁ to ring CY₅₄ may be different from each other.

In one or more embodiments, in Formulae 51A and 51B, R₅₁ to R₅₄ may each independently be hydrogen, deuterium, —F, —Cl, —Br, —I, —SF₅, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₁-C₆₀ alkylthio group, a substituted or unsubstituted C₁-C₆₀ alkylthio group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₇-C₆₀ alkyl aryl group, a substituted or unsubstituted C₇-C₆₀ aryl alkyl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted C₂-C₆₀ alkyl heteroaryl group, a substituted or unsubstituted C₂-C₆₀ heteroaryl alkyl group, a substituted or unsubstituted C₁-C₆₀ heteroaryloxy group, a substituted or unsubstituted C₁-C₆₀ heteroarylthio group, a substituted or unsubstituted monovalent non-aromatic condensed polycyclic group, a substituted or unsubstituted monovalent non-aromatic condensed heteropolycyclic group, —N(Q₁)(Q₂), —Si(Q₃)(Q₄)(Q₅), —Ge(Q₃)(Q₄)(Q₅), —B(Q₆)(Q₇), —P(═O)(Q₈)(Q₉), or —P(Q₈)(Q₉). Q₁ to Q₉ are each as described herein.

In one or more embodiments, in Formulae 51A and 51B, R₅₁ to R₅₄ may each independently be:
hydrogen, deuterium, —F, —Cl, —Br, —I, —SF₅, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a C₁-C₂₀ alkoxy group, or a C₁-C₂₀ alkylthio group;
a C₁-C₂₀ alkyl group, a C₁-C₂₀ alkoxy group, or a C₁-C₂₀ alkylthio group, each substituted with at least one of deuterium, —F, —Cl, —Br, —I, —SF₅, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₁₀ alkyl group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a bicyclo[1.1.1]pentyl group, a bicyclo[2.1.1]hexyl group, a bicyclo[2.2.2]octyl group, a (C₁-C₂₀ alkyl)cyclopentyl group, a (C₁-C₂₀ alkyl)cyclohexyl group, a (C₁-C₂₀ alkyl)cycloheptyl group, a (C₁-C₂₀ alkyl)cyclooctyl group, a (C₁-C₂₀ alkyl)adamantanyl group, a (C₁-C₂₀ alkyl)norbornanyl group, a (C₁-C₂₀ alkyl)norbornenyl group, a (C₁-C₂₀ alkyl)cyclopentenyl group, a (C₁-C₂₀ alkyl)cyclohexenyl group, a (C₁-C₂₀ alkyl)cycloheptenyl group, a (C₁-C₂₀ alkyl)bicyclo[1.1.1]pentyl group, a (C₁-C₂₀ alkyl)bicyclo[2.1.1]hexyl group, a (C₁-C₂₀ alkyl)bicyclo[2.2.2]octyl group, a phenyl group, a (C₁-C₂₀ alkyl)phenyl group, a biphenyl group, a terphenyl group, a naphthyl group, a pyridinyl group, a pyrimidinyl group, or a combination thereof;
a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a bicyclo[1.1.1]pentyl group, a bicyclo[2.1.1]hexyl group, a bicyclo[2.2.2]octyl group, a phenyl group, a (C₁-C₂₀ alkyl)phenyl group, a biphenyl group, a terphenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, a benzoisothiazolyl group, a benzoxazolyl group, a benzoisoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, an imidazopyrimidinyl group, an azacarbazolyl group, an azadibenzofuranyl group or azadibenzothiophenyl group, each unsubstituted or substituted with at least one of deuterium, —F, —Cl, —Br, —I, —SF₅, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₂₀ alkyl group, a (phenyl)C₁-C₁₀ alkyl group, a C₁-C₂₀ alkoxy group, a C₁-C₆₀ alkylthio group, a cyclopentyl group, a cyclohexyl group, a cycloheptyl group, a cyclooctyl group, an adamantanyl group, a norbornanyl group, a norbornenyl group, a cyclopentenyl group, a cyclohexenyl group, a cycloheptenyl group, a bicyclo[1.1.1]pentyl group, a bicyclo[2.1.1]hexyl group, a bicyclo[2.2.2]octyl group, a (C₁-C₂₀ alkyl)cyclopentyl group, a (C₁-C₂₀ alkyl)cyclohexyl group, a (C₁-C₂₀ alkyl)cycloheptyl group, a (C₁-C₂₀ alkyl)cyclooctyl group, a (C₁-C₂₀ alkyl)adamantanyl group, a (C₁-C₂₀ alkyl)norbornanyl group, a (C₁-C₂₀ alkyl)norbornenyl group, a (C₁-C₂₀ alkyl)cyclopentenyl group, a (C₁-C₂₀ alkyl)cyclohexenyl group, a (C₁-C₂₀ alkyl)cycloheptenyl group, a (C₁-C₂₀ alkyl)bicyclo[1.1.1]pentyl group, a (C₁-C₂₀ alkyl)bicyclo[2.1.1]hexyl group, a (C₁-C₂₀ alkyl)bicyclo[2.2.2]octyl group, a phenyl group, a (C₁-C₂₀ alkyl)phenyl group, a biphenyl group, a terphenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a carbazolyl group, a phenanthrolinyl group, a benzimidazolyl group, a benzofuranyl group, a benzothiophenyl group, a benzoisothiazolyl group, a benzoxazolyl group, a benzoisoxazolyl group, a triazolyl group, a tetrazolyl group, an oxadiazolyl group, a triazinyl group, a dibenzofuranyl group, a dibenzothiophenyl group, a benzocarbazolyl group, a dibenzocarbazolyl group, an imidazopyridinyl group, an imidazopyrimidinyl group, an azacarbazolyl group, an azadibenzofuranyl group, an azadibenzothiophenyl group, or a combination thereof; or
—N(Q₁)(Q₂), —Si(Q₃)(Q₄)(Q₅), —Ge(Q₃)(Q₄)(Q₅), —B(Q₆)(Q₇), —P(═O)(Q₈)(Q₉), or —P(Q₈)(Q₉), and
Q₁ to Q₉ may each independently be:
—CH₃, —CD₃, —CD₂H, —CDH₂, —CH₂CH₃, —CH₂CD₃, —CH₂CD₂H, —CH₂CDH₂, —CHDCH₃, —CHDCD₂H, —CHDCDH₂, —CHDCD₃, —CD₂CD₃, —CD₂CD₂H, or —CD₂CDH₂; or
an n-propyl group, an isopropyl group, an n-butyl group, a sec-butyl group, an isobutyl group, a tert-butyl group, an n-pentyl group, a tert-pentyl group, an neopentyl group, an isopentyl group, a sec-pentyl group, a 3-pentyl group, a sec-isopentyl group, a phenyl group, a biphenyl group, or a naphthyl group, each unsubstituted or substituted with at least one of deuterium, a C₁-C₆₀ alkyl group, a phenyl group, or a combination thereof.

In one or more embodiments, R₅₁ to R₅₄ may each independently be:
hydrogen, deuterium, —F, or a cyano group;
a C₁-C₂₀ alkyl group unsubstituted or substituted with at least one of deuterium, a cyano group, a C₃-C₁₀ cycloalkyl group, a deuterated C₃-C₁₀ cycloalkyl group, a fluorinated C₃-C₁₀ cycloalkyl group, a (C₁-C₂₀ alkyl)C₃-C₁₀ cycloalkyl group, a C₁-C₁₀ heterocycloalkyl group, a deuterated C₁-C₁₀ heterocycloalkyl group, a fluorinated C₁-C₁₀ heterocycloalkyl group, a (C₁-C₂₀ alkyl)C₁-C₁₀ heterocycloalkyl group, a phenyl group, a deuterated a phenyl group, a fluorinated a phenyl group, a (C₁-C₂₀ alkyl)phenyl group, a biphenyl group, a deuterated a biphenyl group, a fluorinated a biphenyl group, a (C₁-C₂₀ alkyl)biphenyl group, a dibenzofuranyl group, a deuterated a dibenzofuranyl group, a fluorinated a dibenzofuranyl group, a (C₁-C₂₀ alkyl)dibenzofuranyl group, a dibenzothiophenyl group, a deuterated a dibenzothiophenyl group, a fluorinated a dibenzothiophenyl group, a (C₁-C₂₀ alkyl)dibenzothiophenyl group, or a combination thereof;
a C₃-C₁₀ cycloalkyl group, a C₁-C₁₀ heterocycloalkyl group, a phenyl group, or a biphenyl group, each unsubstituted or substituted with at least one of deuterium, a cyano group, a C₁-C₂₀ alkyl group, a deuterated C₁-C₂₀ alkyl group, fluorinated C₁-C₂₀ alkyl group, a C₁-C₂₀ alkoxy group, a C₁-C₆₀ alkylthio group, a deuterated C₁-C₂₀ alkoxy group, a fluorinated C₁-C₂₀ alkoxy group, a C₃-C₁₀ cycloalkyl group, a deuterated C₃-C₁₀ cycloalkyl group, a fluorinated C₃-C₁₀ cycloalkyl group, a (C₁-C₂₀ alkyl)C₃-C₁₀ cycloalkyl group, a C₁-C₁₀ heterocycloalkyl group, a deuterated C₁-C₁₀ heterocycloalkyl group, a fluorinated C₁-C₁₀ heterocycloalkyl group, a (C₁-C₂₀ alkyl)C₁-C₁₀ heterocycloalkyl group, a phenyl group, a deuterated phenyl group, a fluorinated a phenyl group, a (C₁-C₂₀ alkyl)phenyl group, a biphenyl group, a deuterated biphenyl group, a fluorinated a biphenyl group, a (C₁-C₂₀ alkyl)biphenyl group, a dibenzofuranyl group, a deuterated dibenzofuranyl group, a fluorinated a dibenzofuranyl group, a (C₁-C₂₀ alkyl)dibenzofuranyl group, a dibenzothiophenyl group, a deuterated dibenzothiophenyl group, a fluorinated a dibenzothiophenyl group, a (C₁-C₂₀ alkyl)dibenzothiophenyl group, or a combination thereof; or
—Si(Q₃)(Q₄)(Q₅) or —Ge(Q₃)(Q₄)(05), wherein O₃ to O₅ may be as defined herein.

In Formulae 51A and 51B, b51 to b54 indicate the number of R₅₁ to R₅₄, respectively, and may each independently be an integer from 0 to 20. When b51 is 2 or greater, two or more of R₅₁ may be identical to or different from each other, when b52 is 2 or greater, two or more of R₅₂ may be identical to or different from each other, when b53 is 2 or greater, two or more of R₅₃ may be identical to or different from each other, and when b54 is 2 or greater, two or more of R₅₄ may be identical to or different from each other. For example, b51 to b54 may each independently be an integer from 0 to 8.

In one or more embodiments, the phosphorescent dopant may include at least one of Compounds P1 to P52, but embodiments are not limited thereto:

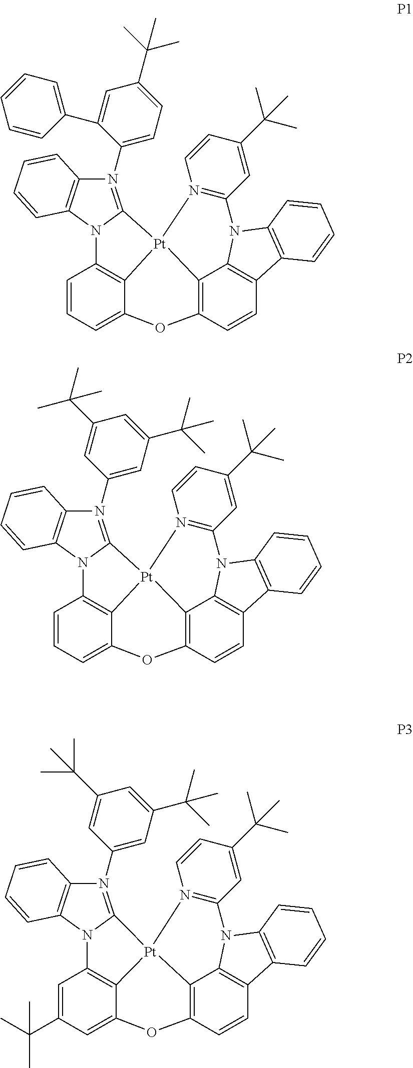

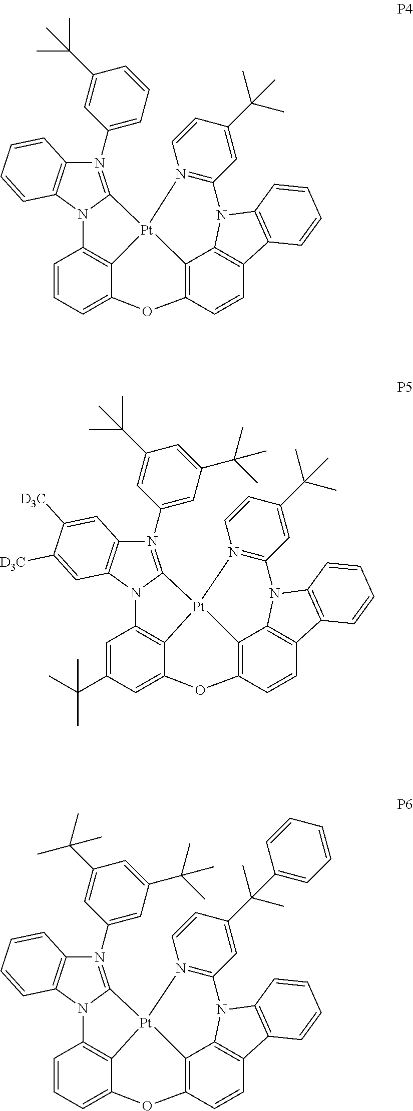

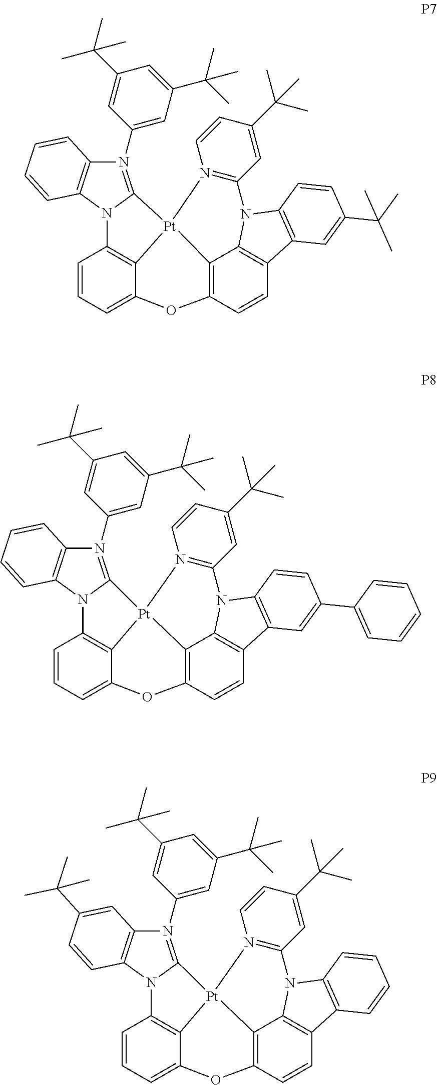

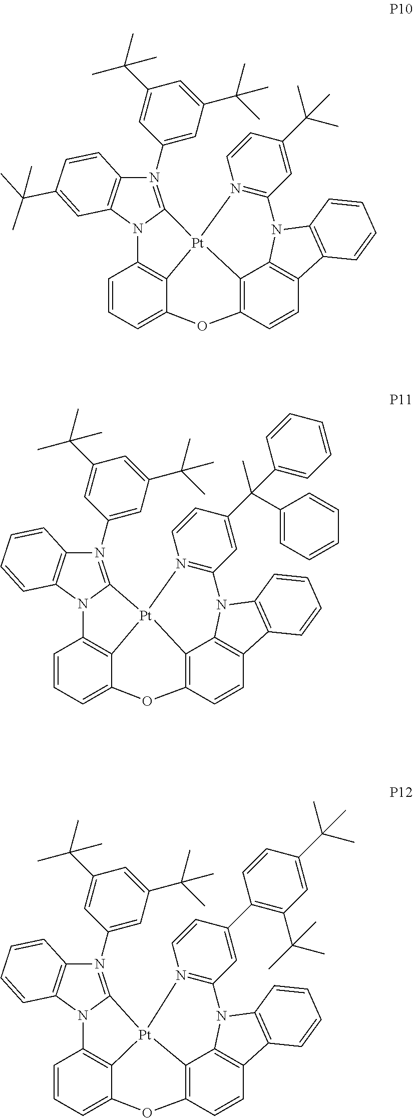

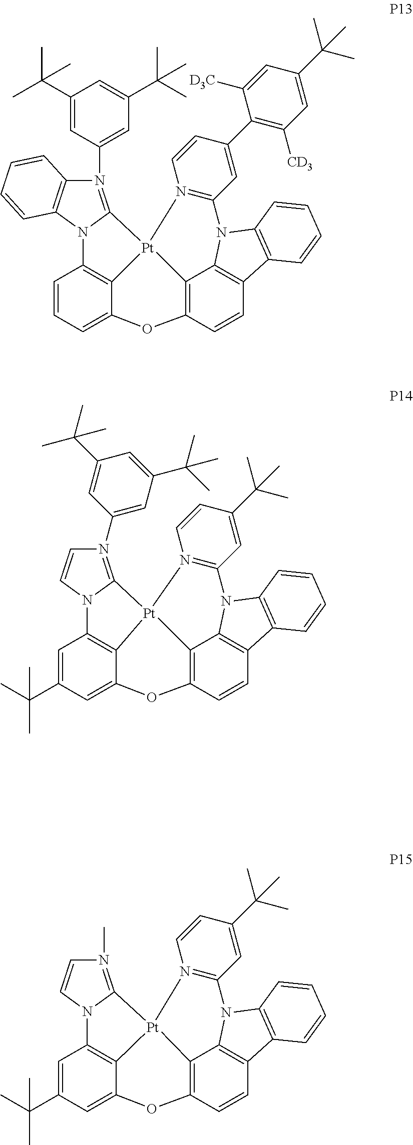

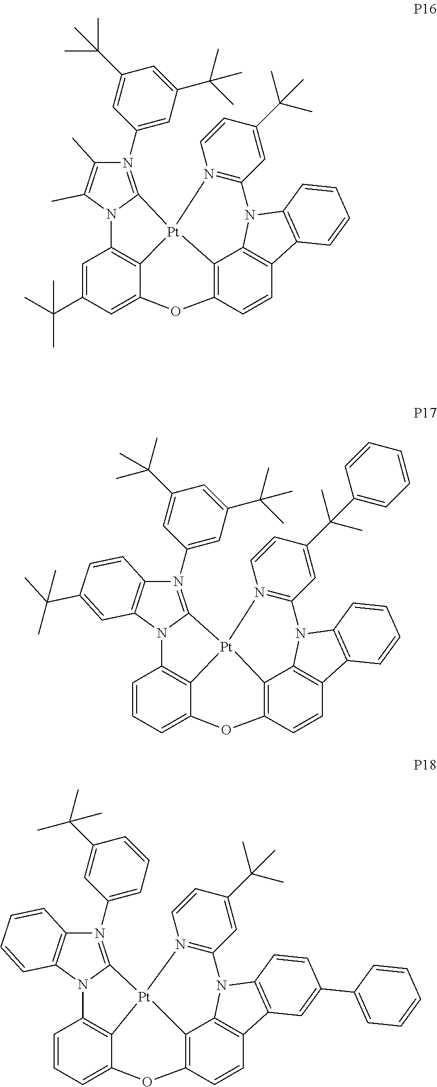

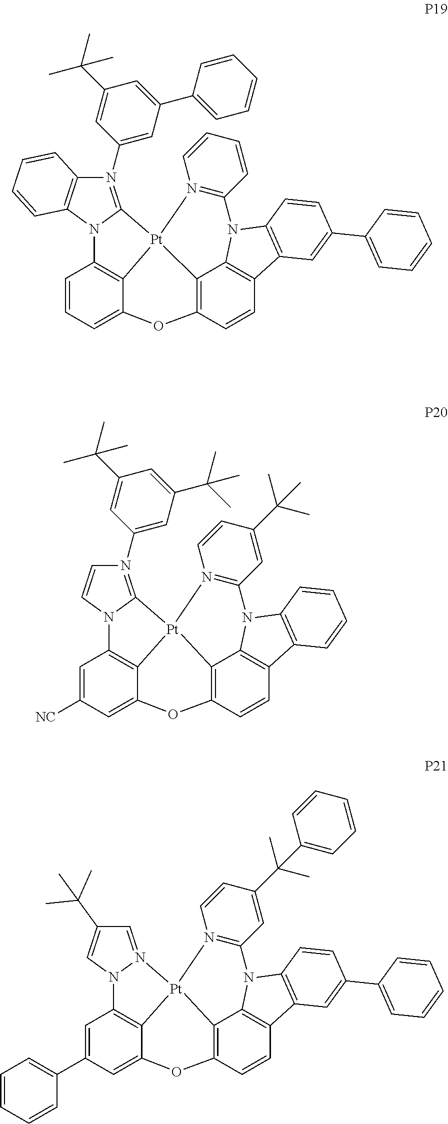

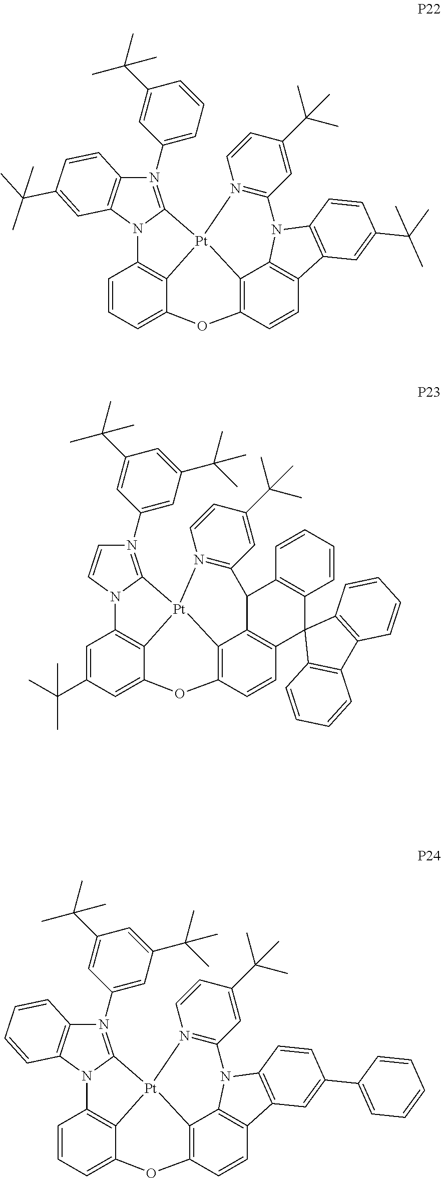

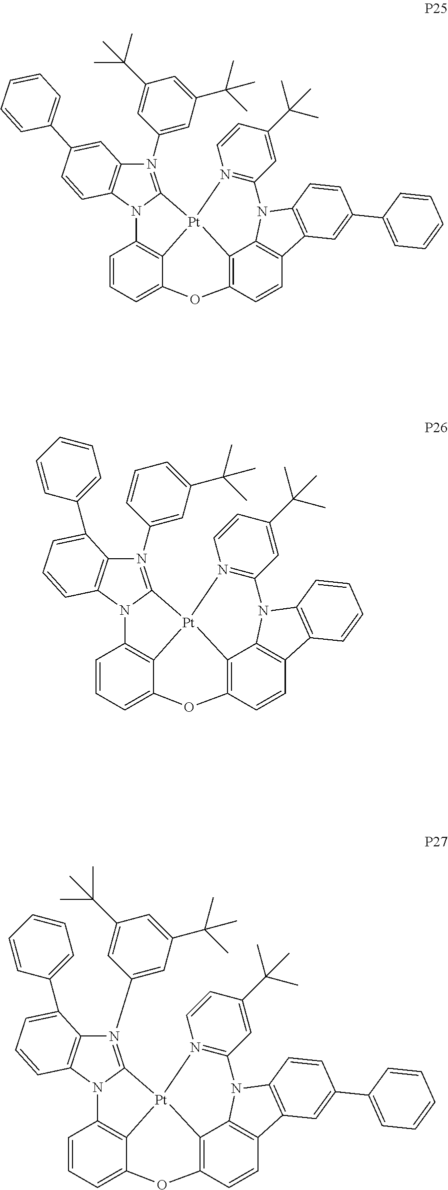

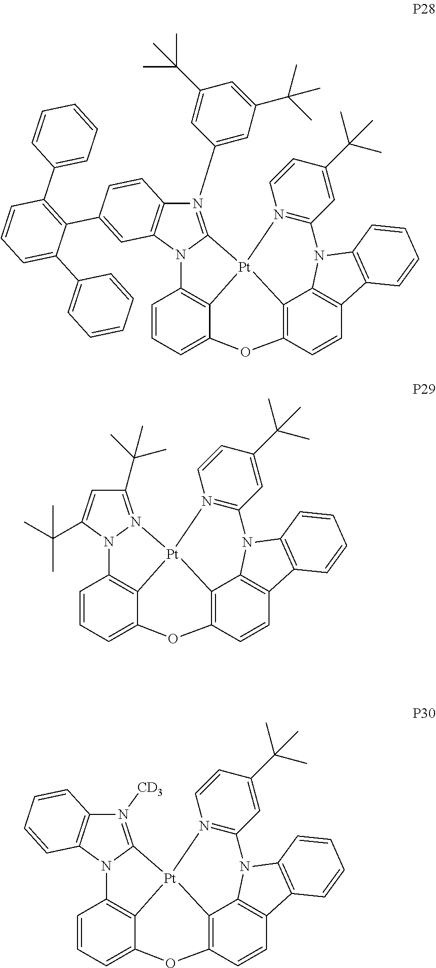

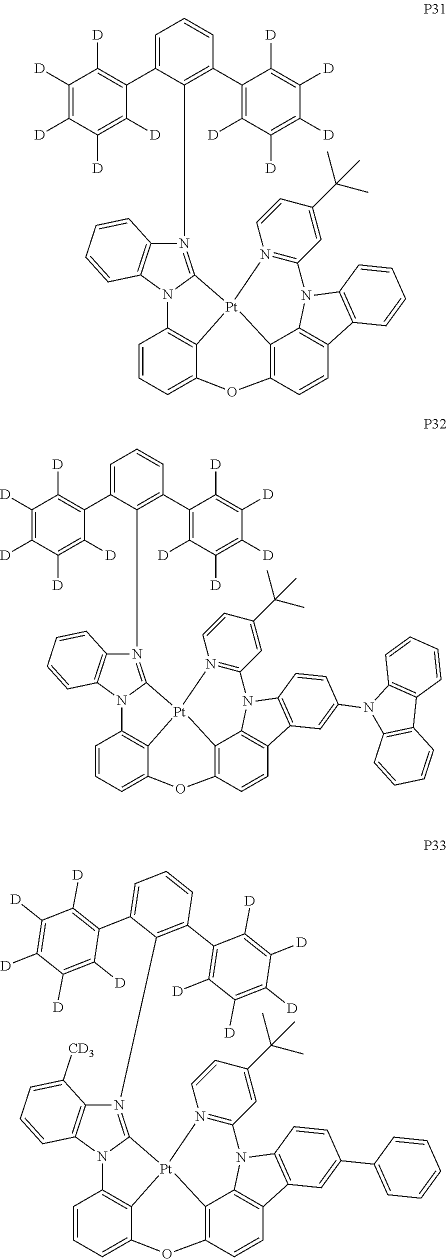

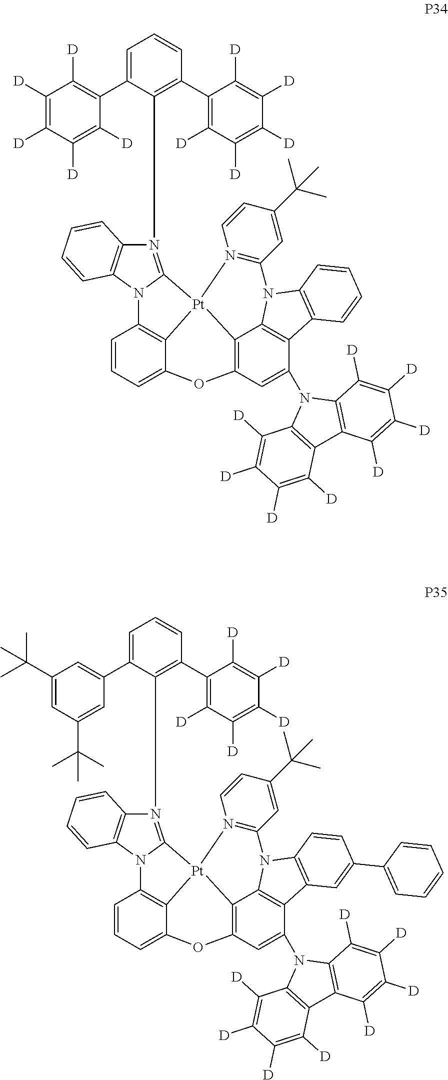

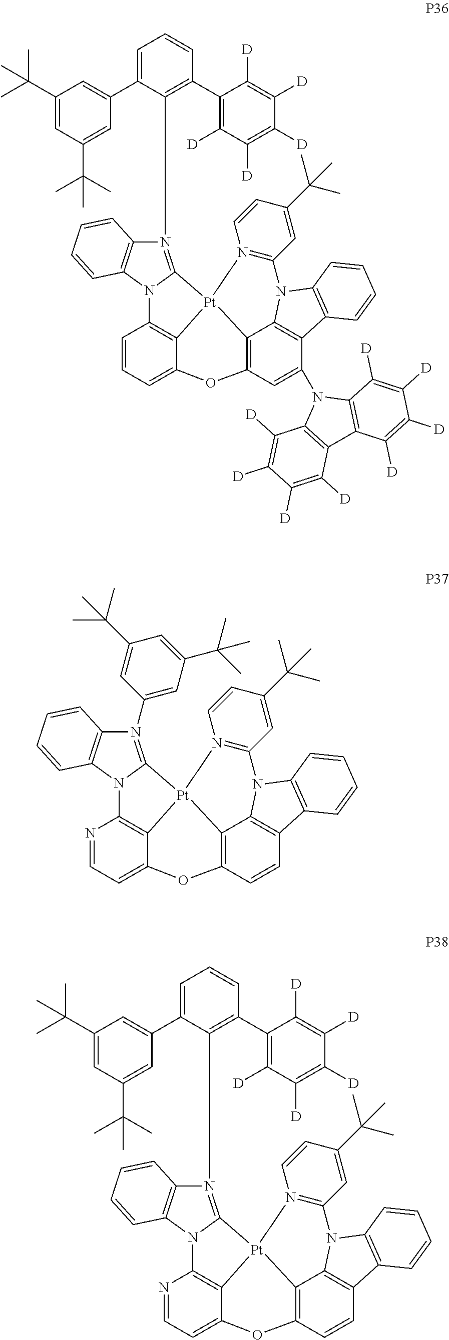

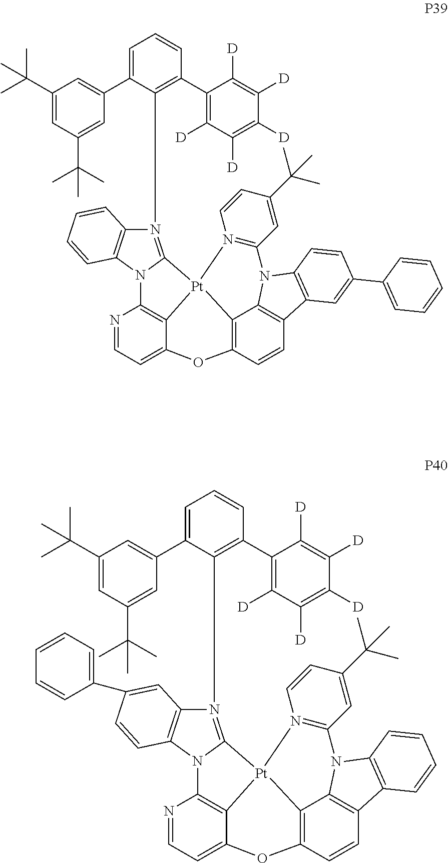

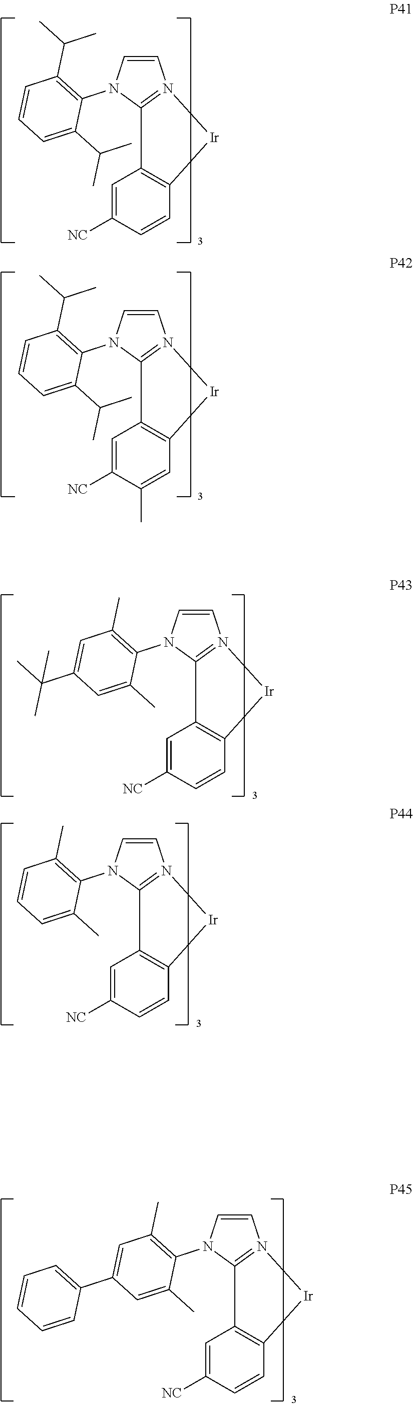

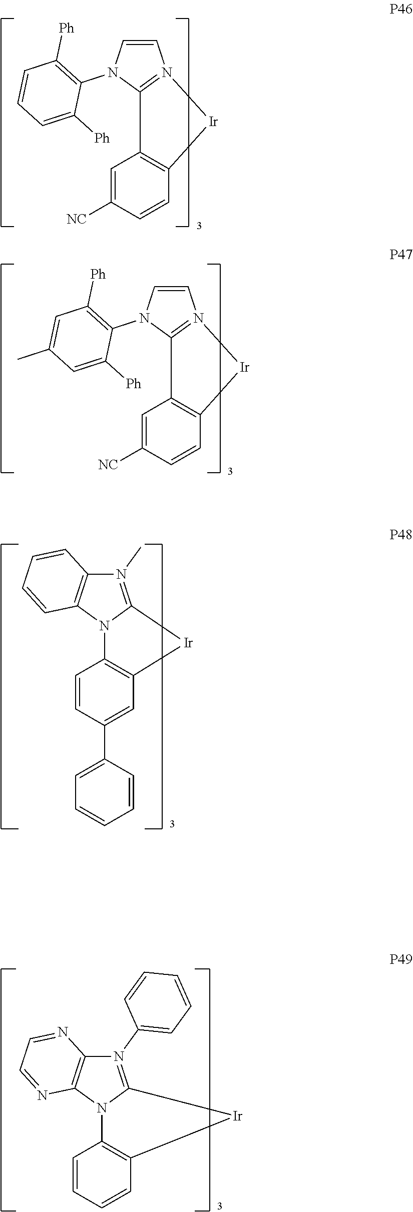

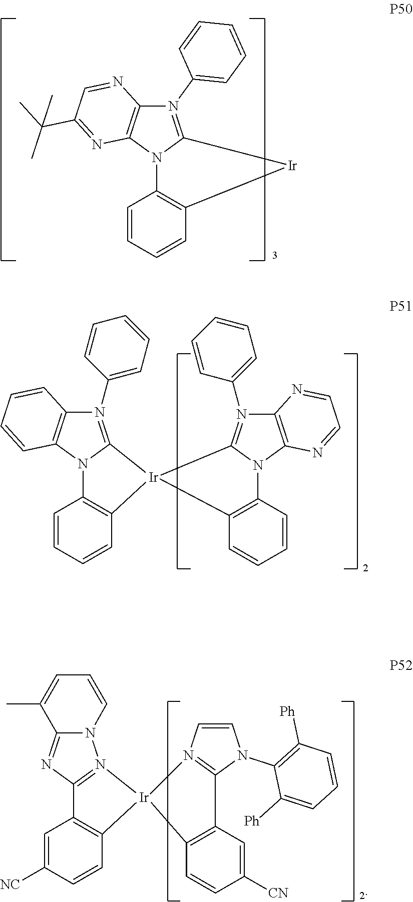

Without wishing to be bound to theory, when the phosphorescent dopant is selected from at least one of Compounds P1 to P52, exciplex formation with the aforementioned host compound may be facilitated. For example, the phosphorescent dopant may have, by including bulky substituents (for example, a tert-butyl group, a cumyl group, or the like), an energy level close to that of an energy level of the host compound, and thus exciplex formation may be facilitated. In the case of the phosphorescent dopant, an energy level gap between a LUMO level of the electron-transporting host and a HOMO level of the phosphorescent dopant may be reduced, thereby facilitating the exciplex formation.

### Fluorescent Dopant

In one or more embodiments, the emission layer may further include a fluorescent dopant. For example, the fluorescent dopant may be a thermally activated delayed fluorescence dopant and/or a blue dopant.

The fluorescent dopant may have a difference between a triplet (Ti) energy level and a singlet (Si) energy level of less than or equal to about 0.4 eV.

For example, the fluorescent dopant may be a thermally activated delayed fluorescence (TADF) dopant and/or a blue dopant.

In one or more embodiments, the fluorescent dopant may be a luminescence emitter that may emit a light by receiving excitons from the exciplex of the host and the phosphorescent dopant according to one or more embodiments so that the received excitons transition to a ground state.

In one or more embodiments, the fluorescent dopant may be at least one compound represented by Formula 41, but embodiments are not limited thereto:

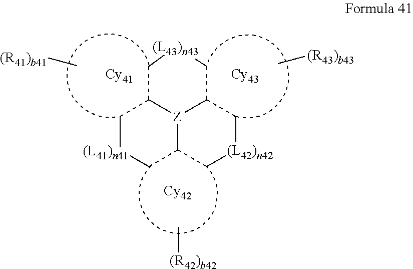

wherein, in Formula 41,
Z may be B or N,
ring CY₄₁ to ring CY₄₃ may each independently be a C₅-C₃₀ carbocyclic group or a C₁-C₃₀ heterocyclic group,
L₄₁ may be *—N(R₄₄)—*′, *—B(R₄₄)—*′, *—P(R₄₄)—*′, *—C(R₄₄)(R₄₅)—*′, *—Si(R₄₄)(R₄₅)—*′, *—Ge(R₄₄)(R₄₅)—*′, *—O—*′, *—S—*′, *—Se—*′, *—C(═)—*′, or *—S(═O)₂—*′,
L₄₂ may be *—N(R₄₆)—*′, *—B(R₄₆)—*′, *—P(R₄₆)—*′, *—C(R₄₆)(R₄₇)—*′, *—Si(R₄₆)(R₄₇)—*′ *—Ge(R₄₆)(R₄₇)—*′, *—O*′, *—S—*′, *—Se—*′, *—C(═)—*′, or *—S(═O)₂—*′,
L₄₃ may be *—N(R₄₈)—*′, *—B(R₄₈)—*′, *—P(R₄₈)—*′, *—C(R₄₈)(R₄₉)—*′, *—Si(R₄₈)(R₄₉)—*′ *—Ge(R₄₈)(R₄₉)—*′, *—0*′, *—S—*′, *—Se—*′, *—C(═)—*′, or *—S(═O)₂—*′,
* and *′ each indicate a binding site to a neighboring atom,
n41 to n43 may each independently be 0 or 1,
when n41 is 0, L₄₁ is absent, when n42 is 0, L₄₂ is absent, and when n43 is 0, L₄₃ is absent,
R₄₁ to R₄₉ may each independently be hydrogen, deuterium, —F, —Cl, —Br, —I, —SF₅, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₁-C₆₀ alkylthio group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₇-C₆₀ alkyl aryl group, a substituted or unsubstituted C₇-C₆₀ aryl alkyl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted C₂-C₆₀ alkyl heteroaryl group, a substituted or unsubstituted C₂-C₆₀ heteroaryl alkyl group, a substituted or unsubstituted C₁-C₆₀ heteroaryloxy group, a substituted or unsubstituted C₁-C₆₀ heteroarylthio group, a substituted or unsubstituted monovalent non-aromatic condensed polycyclic group, a substituted or unsubstituted monovalent non-aromatic condensed heteropolycyclic group, —N(Q₁)(Q₂), —Si(Q₃)(Q₄)(Q₅), —Ge(Q₃)(Q₄)(Q₅), —B(Q₆)(Q₇), —P(═O)(Q₈)(Q₉), or —P(Q₈)(Q₉),
b41 to b43 may each independently be an integer from 1 to 20,
at least two of R₄₁ may optionally be bonded to each other to form a C₅-C₃₀ carbocyclic group unsubstituted or substituted with at least one R_{10b}, or a C₁-C₃₀ heterocyclic group unsubstituted or substituted with at least one R_{10b},
at least two of R₄₂ may optionally be bonded to each other to form a C₅-C₃₀ carbocyclic group unsubstituted or substituted with at least one R_{10b}, or a C₁-C₃₀ heterocyclic group unsubstituted or substituted with at least one R_{10b},
at least two of R₄₃ may optionally be bonded to each other to form a C₅-C₃₀ carbocyclic group unsubstituted or substituted with at least one R_{10b}, or a C₁-C₃₀ heterocyclic group unsubstituted or substituted with at least one R_{10b},
two or more of R₄₁ to R₄₉ may optionally be bonded to each other to form a C₅-C₃₀ carbocyclic group unsubstituted or substituted with at least one R_{10b}, or a C₁-C₃₀ heterocyclic group unsubstituted or substituted with at least one R_{10b},
R_{10b} may be as described herein in connection with R₄₁,
at least one substituent of the substituted C₅-C₃₀ carbocyclic group, the substituted C₁-C₃₀ heterocyclic group, the substituted C₁-C₆₀ alkyl group, the substituted C₂-C₆₀ alkenyl group, the substituted C₂-C₆₀ alkynyl group, the substituted C₁-C₆₀ alkoxy group, the substituted C₁-C₆₀ alkylthio group, the substituted C₃-C₁₀ cycloalkyl group, the substituted C₁-C₁₀ heterocycloalkyl group, the substituted C₃-C₁₀ cycloalkenyl group, the substituted C₁-C₁₀ heterocycloalkenyl group, the substituted C₆-C₆₀ aryl group, the substituted C₇-C₆₀ alkyl aryl group, the substituted C₇-C₆₀ aryl alkyl group, the substituted C₆-C₆₀ aryloxy group, the substituted C₆-C₆₀ arylthio group, the substituted C₁-C₆₀ heteroaryl group, the substituted C₂-C₆₀ alkyl heteroaryl group, the substituted C₂-C₆₀ heteroaryl alkyl group, the substituted C₁-C₆₀ heteroaryloxy group, the substituted C₁-C₆₀ heteroarylthio group, the substituted monovalent non-aromatic condensed polycyclic group, and the substituted monovalent non-aromatic condensed heteropolycyclic group may be:
deuterium, —F, —Cl, —Br, —I, —SF₅, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, or a C₁-C₆₀ alkylthio group;
a C₁-C₆₀ alkyl group, a C₂-C₆₀ alkenyl group, a C₂-C₆₀ alkynyl group, a C₁-C₆₀ alkoxy group, or a C₁-C₆₀ alkylthio group, each substituted with at least one of deuterium, —F, —Cl, —Br, —I, —SF₅, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₇-C₆₀ alkyl aryl group, a C₆-C₆₀ aryloxy group, a C₆-C₆₀ arylthio group, a C₁-C₆₀ heteroaryl group, a C₂-C₆₀ alkyl heteroaryl group, a C₁-C₆₀ heteroaryloxy group, a C₁-C₆₀ heteroarylthio group, a monovalent non-aromatic condensed polycyclic group, a monovalent non-aromatic condensed heteropolycyclic group, —N(Q₁₁)(Q₁₂), —Si(Q₁₃)(Q₁₄)(Q₁₅), —Ge(Q₁₃)(Q₁₄)(Q₁₅), —B(Q₁₆)(Q₁₇), —P(═O)(Q₁₈)(Q₁₉), —P(Q₁₈)(Q₁₉), or a combination thereof;
a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₇-C₆₀ alkyl aryl group, a C₆-C₆₀ aryloxy group, a C₆-C₆₀ arylthio group, a C₁-C₆₀ heteroaryl group, a C₂-C₆₀ alkyl heteroaryl group, a C₁-C₆₀ heteroaryloxy group, a C₁-C₆₀ heteroarylthio group, a monovalent non-aromatic condensed polycyclic group, or a monovalent non-aromatic condensed heteropolycyclic group;
a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₇-C₆₀ alkyl aryl group, a C₆-C₆₀ aryloxy group, a C₆-C₆₀ arylthio group, a C₁-C₆₀ heteroaryl group, a C₂-C₆₀ alkyl heteroaryl group, a C₁-C₆₀ heteroaryloxy group, a C₁-C₆₀ heteroarylthio group, a monovalent non-aromatic condensed polycyclic group, or a monovalent non-aromatic condensed heteropolycyclic group, each substituted with at least one of deuterium, —F, —Cl, —Br, —I, —SF₅, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₁-C₆₀ alkylthio group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₇-C₆₀ alkyl aryl group, a C₇-C₆₀ aryl alkyl group, a C₆-C₆₀ aryloxy group, a C₆-C₆₀ arylthio group, a C₁-C₆₀ heteroaryl group, a C₂-C₆₀ alkyl heteroaryl group, a C₂-C₆₀ heteroaryl alkyl group, a C₁-C₆₀ heteroaryloxy group, a C₁-C₆₀ heteroarylthio group, a monovalent non-aromatic condensed polycyclic group, a monovalent non-aromatic condensed heteropolycyclic group, —N(Q₂₁)(Q₂₂), —Si(Q₂₃)(Q₂₄)(Q₂₅), —Ge(Q₂₃)(Q₂₄)(Q₂₅), —B(Q₂₆)(Q₂₇), —P(═O)(Q₂₈)(Q₂₉), —P(Q₂₈)(Q₂₉), or a combination thereof; or
—N(Q₃₁)(Q₃₂), —Si(Q₃₃)(Q₃₄)(Q₃₅), —Ge(Q₃₃)(Q₃₄)(Q₃₅), —B(Q₃₆)(Q₃₇), —P(═O)(Q₃₈)(Q₃₉), or —P(Q₃₈)(Q₃₉), and
Q₁ to Q₉, Q₁₁ to Q₁₉, Q₂₁ to Q₂₉, and Q₃₁ to Q₃₉ may each independently be hydrogen, deuterium, —F, —Cl, —Br, —I, —SF₅, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₁-C₆₀ alkylthio group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₇-C₆₀ alkyl aryl group, a substituted or unsubstituted C₇-C₆₀ aryl alkyl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted C₂-C₆₀ alkyl heteroaryl group, a substituted or unsubstituted C₂-C₆₀ heteroaryl alkyl group, a substituted or unsubstituted C₁-C₆₀ heteroaryloxy group, a substituted or unsubstituted C₁-C₆₀ heteroarylthio group, a substituted or unsubstituted monovalent non-aromatic condensed polycyclic group, or a substituted or unsubstituted monovalent non-aromatic condensed heteropolycyclic group.

In one or more embodiments, R₄₁ to R₄₉ may each independently be:
hydrogen, deuterium, —F, —Cl, —Br, —I, —SF₅, a hydroxyl group, a cyano group, a nitro group, an amino group, a C₁-C₂₀ alkyl group, a C₁-C₂₀ alkoxy group, or a C₁-C₂₀ alkylthio group;
a C₁-C₆₀ alkyl group, a C₁-C₆₀ alkoxy group, or a C₁-C₂₀ alkylthio group, each substituted with at least one of deuterium, —F, —Cl, —Br, —I, —SF₅, a hydroxyl group, a cyano group, a nitro group, an amino group, a phenyl group, a biphenyl group, a naphthyl group, a fluorenyl group, a phenanthrenyl group, an anthracenyl group, a fluoranthenyl group, a triphenylenyl group, a pyrenyl group, a chrysenyl group, or a combination thereof; or
a cyclopentyl group, a cyclohexyl group, a cycloheptyl group, a cyclopentenyl group, a cyclohexenyl group, a cycloheptenyl group, a phenyl group, a biphenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a phenanthrolinyl group, a benzimidazolyl group, a benzofuranyl group, a benzothiophenyl group, a benzoisothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, or a carbazolyl group, each unsubstituted or substituted with at least one of deuterium, —F, —Cl, —Br, —I, —SF₅, a hydroxyl group, a cyano group, a nitro group, an amino group, a C₁-C₆₀ alkyl group, a C₁-C₆₀ alkoxy group, a C₁-C₆₀ alkylthio group, a cyclopentyl group, a cyclohexyl group, a cycloheptyl group, a cyclopentenyl group, a cyclohexenyl group, a cycloheptenyl group, a phenyl group, a biphenyl group, a naphthyl group, a fluorenyl group, a phenanthrenyl group, an anthracenyl group, a fluoranthenyl group, a triphenylenyl group, a pyrenyl group, a chrysenyl group, a pyrrolyl group, a thiophenyl group, a furanyl group, an imidazolyl group, a pyrazolyl group, a thiazolyl group, an isothiazolyl group, an oxazolyl group, an isoxazolyl group, a pyridinyl group, a pyrazinyl group, a pyrimidinyl group, a pyridazinyl group, an isoindolyl group, an indolyl group, an indazolyl group, a purinyl group, a quinolinyl group, an isoquinolinyl group, a benzoquinolinyl group, a quinoxalinyl group, a quinazolinyl group, a cinnolinyl group, a phenanthrolinyl group, a benzimidazolyl group, a benzofuranyl group, a benzothiophenyl group, a benzoisothiazolyl group, a benzoxazolyl group, an isobenzoxazolyl group, a triazolyl group, a tetrazolyl group, an oxadiazolyl group, a triazinyl group, a dibenzofuranyl group, a dibenzothiophenyl group, a carbazolyl group, or a combination thereof.

In one or more embodiments, the compound represented by Formula 41 may be represented by at least one of Formulae 41-1 to 41-9:

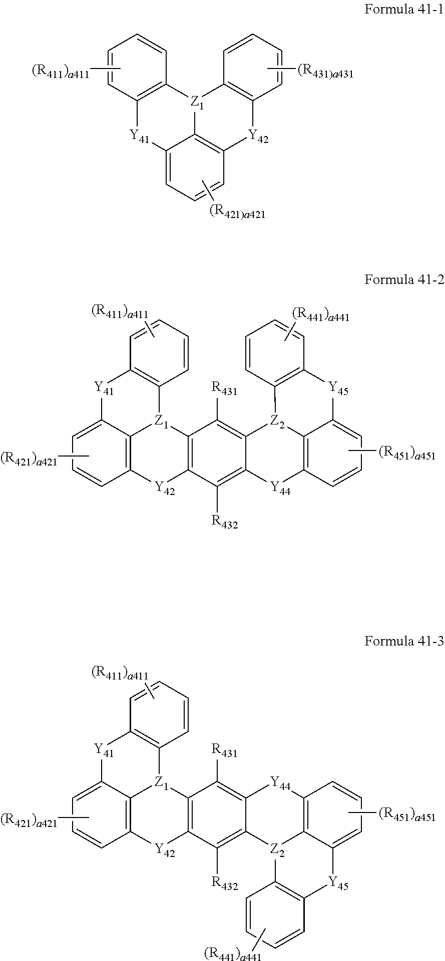

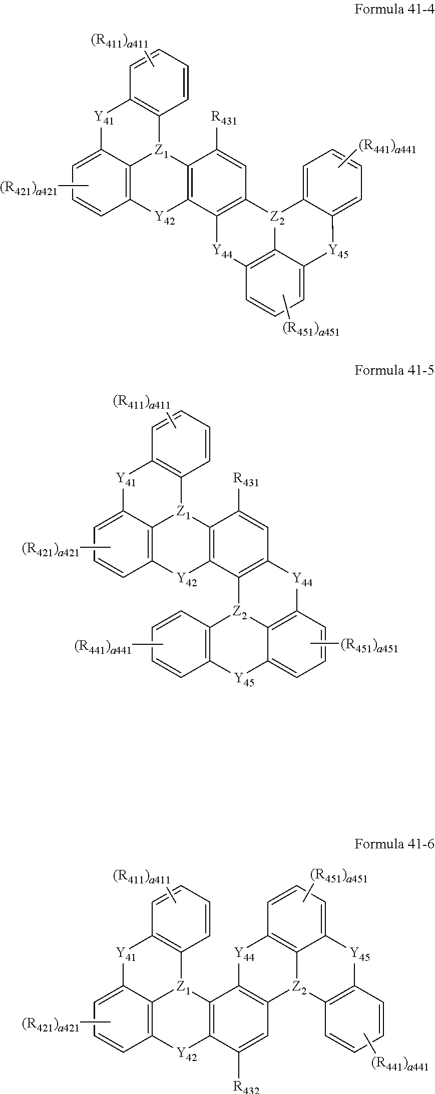

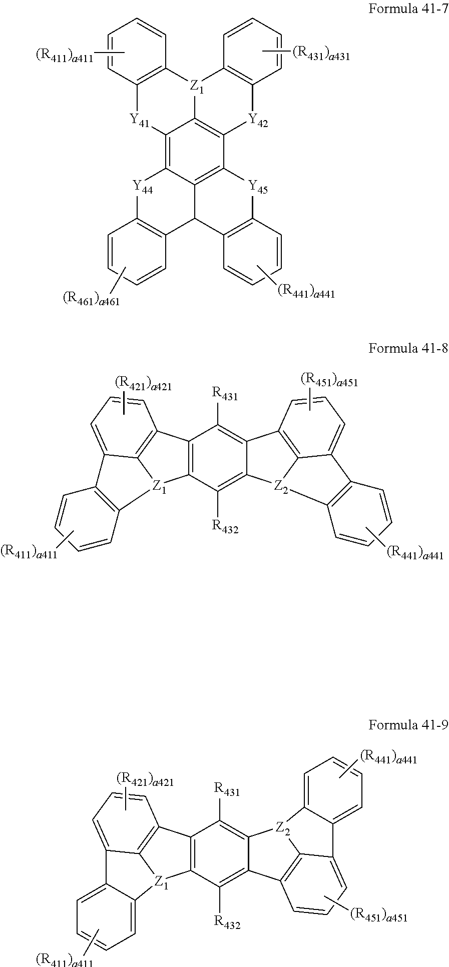

wherein, in Formulae 41-1 to 41-9,
Z₁ and Z₂ may each be as described in connection with Z in Formula 41,
Y₄₁ and Y₄₂ may each be as described in connection with L₄₁ and L₄₂ in Formula 41,
Y₄₄ and Y₄₅ may be as described in connection with L₄₁ and L₄₂ in Formula 41, respectively,
R₄₁1 may be as described in connection with R₄₁ in Formula 41, R₄₂₁ may be as described in connection with R₄₂ in Formula 41, R₄₃₁ and R₄₃₂ may each be as described in connection with R₄₃ in Formula 41, R₄₄₁ may be as described in connection with R₄₁ in Formula 41, R₄₅₁ may be as described in connection with R₄₂ in Formula 41, and R₄₆₁ may be as described in connection with R₄₃ in Formula 41,
a411 may be an integer from 0 to 4,
a421 may be an integer from 0 to 3,
a431 may be an integer from 0 to 4,
a441 may be an integer from 0 to 4,
a451 may be an integer from 0 to 3, and
a461 may be an integer from 0 to 4.

In one or more embodiments, the fluorescent dopant may be at least one of Compounds D1 to D30, but embodiments are not limited thereto:

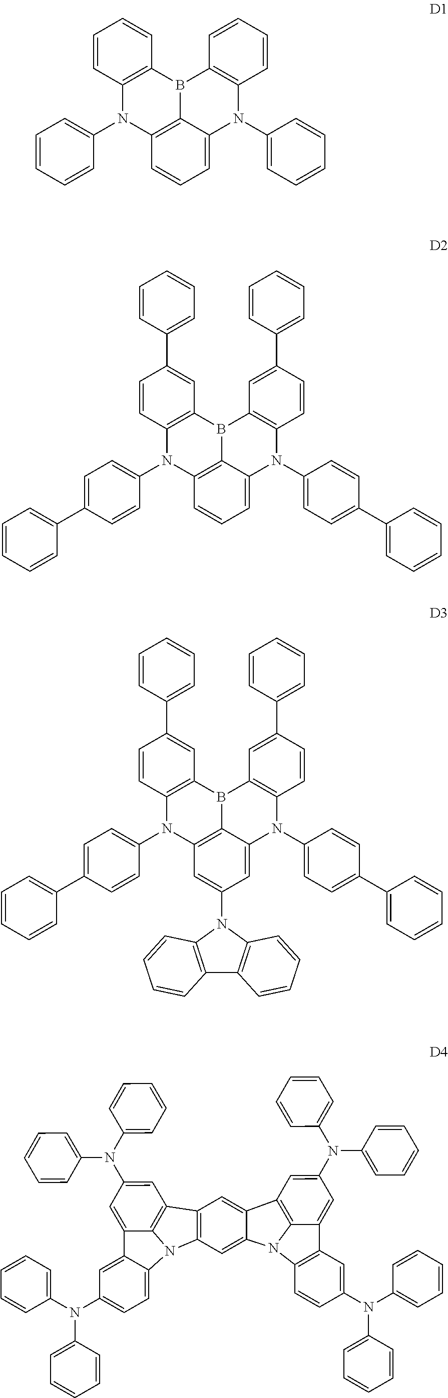 [SMILES: c1ccc(N2c3ccccc3B3c4ccccc4N(c4ccccc4)c4cccc2c43)cc1 ; c1ccc(-c2ccc(N3c4ccc(-c5ccccc5)cc4B4c5cc(-c6ccccc6)ccc5N(c5ccc(-c6ccccc6)cc5)c5cccc3c54)cc2)cc1 ; c1ccc(-c2ccc(N3c4ccc(-c5ccccc5)cc4B4c5cc(-c6ccccc6)ccc5N(c5ccc(-c6ccccc6)cc5)c5cc(-n6c7ccccc7c7ccccc76)cc3c54)cc2)cc1 ; c1ccc(N(c2ccccc2)c2ccc3c(c2)c2cc(N(c4ccccc4)c4ccccc4)cc4c5cc6c7cc(N(c8ccccc8)c8ccccc8)cc8c9cc(N(c%10ccccc%10)c%10ccccc%10)ccc9n(c6cc5n3c24)c87)cc1]

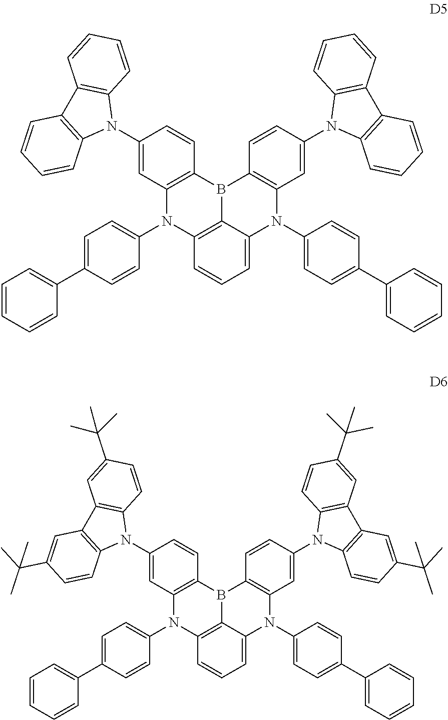 [SMILES: c1ccc(-c2ccc(N3c4cc(-n5c6ccccc6c6ccccc65)ccc4B4c5ccc(-n6c7ccccc7c7ccccc76)cc5N(c5ccc(-c6ccccc6)cc5)c5cccc3c54)cc2)cc1 ; CC(C)(C)c1ccc2c(c1)c1cc(C(C)(C)C)ccc1n2-c1ccc2c(c1)N(c1ccc(-c3ccccc3)cc1)c1cccc3c1B2c1ccc(-n2c4ccc(C(C)(C)C)cc4c4cc(C(C)(C)C)ccc42)cc1N3c1ccc(-c2ccccc2)cc1]

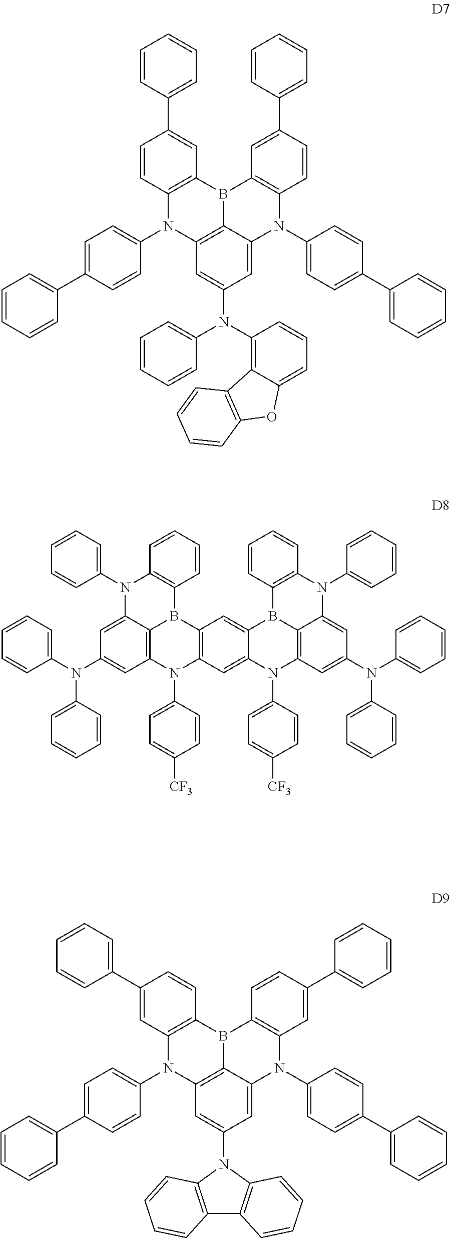 [SMILES: c1ccc(-c2ccc(N3c4ccc(-c5ccccc5)cc4B4c5cc(-c6ccccc6)ccc5N(c5ccc(-c6ccccc6)cc5)c5cc(N(c6ccccc6)c6cccc7oc8ccccc8c67)cc3c54)cc2)cc1 ; FC(F)(F)c1ccc(N2c3cc4c(cc3B3c5ccccc5N(c5ccccc5)c5cc(N(c6ccccc6)c6ccccc6)cc2c53)B2c3ccccc3N(c3ccccc3)c3cc(N(c5ccccc5)c5ccccc5)cc(c32)N4c2ccc(C(F)(F)F)cc2)cc1 ; c1ccc(-c2ccc(N3c4cc(-c5ccccc5)ccc4B4c5ccc(-c6ccccc6)cc5N(c5ccc(-c6ccccc6)cc5)c5cc(-n6c7ccccc7c7ccccc76)cc3c54)cc2)cc1]

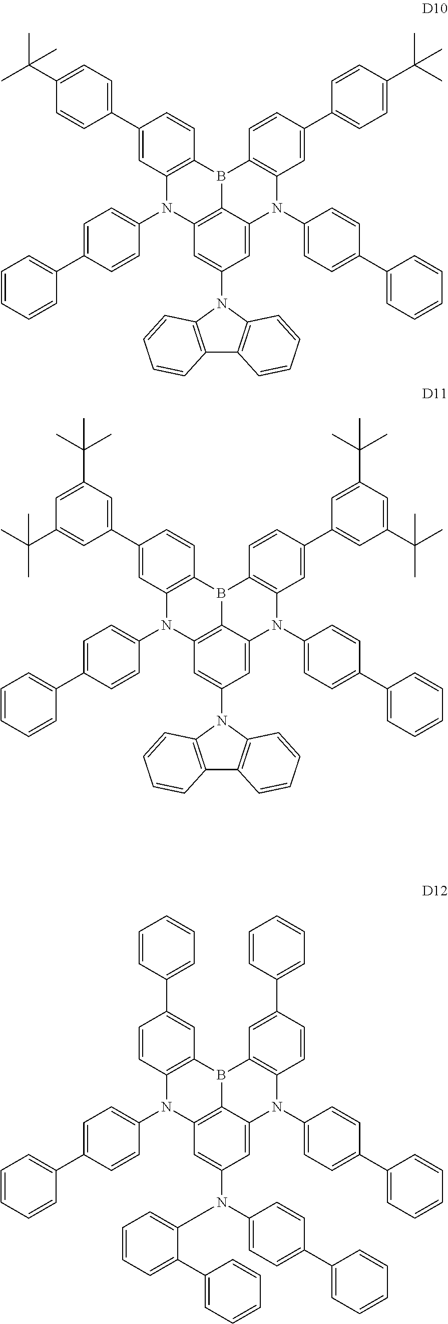 [SMILES: CC(C)(C)c1ccc(-c2ccc3c(c2)N(c2ccc(-c4ccccc4)cc2)c2cc(-n4c5ccccc5c5ccccc54)cc4c2B3c2ccc(-c3ccc(C(C)(C)C)cc3)cc2N4c2ccc(-c3ccccc3)cc2)cc1 ; CC(C)(C)c1cc(-c2ccc3c(c2)N(c2ccc(-c4ccccc4)cc2)c2cc(-n4c5ccccc5c5ccccc54)cc4c2B3c2ccc(-c3cc(C(C)(C)C)cc(C(C)(C)C)c3)cc2N4c2ccc(-c3ccccc3)cc2)cc(C(C)(C)C)c1 ; c1ccc(-c2ccc(N(c3cc4c5c(c3)N(c3ccc(-c6ccccc6)cc3)c3ccc(-c6ccccc6)cc3B5c3cc(-c5ccccc5)ccc3N4c3ccc(-c4ccccc4)cc3)c3ccccc3-c3ccccc3)cc2)cc1]

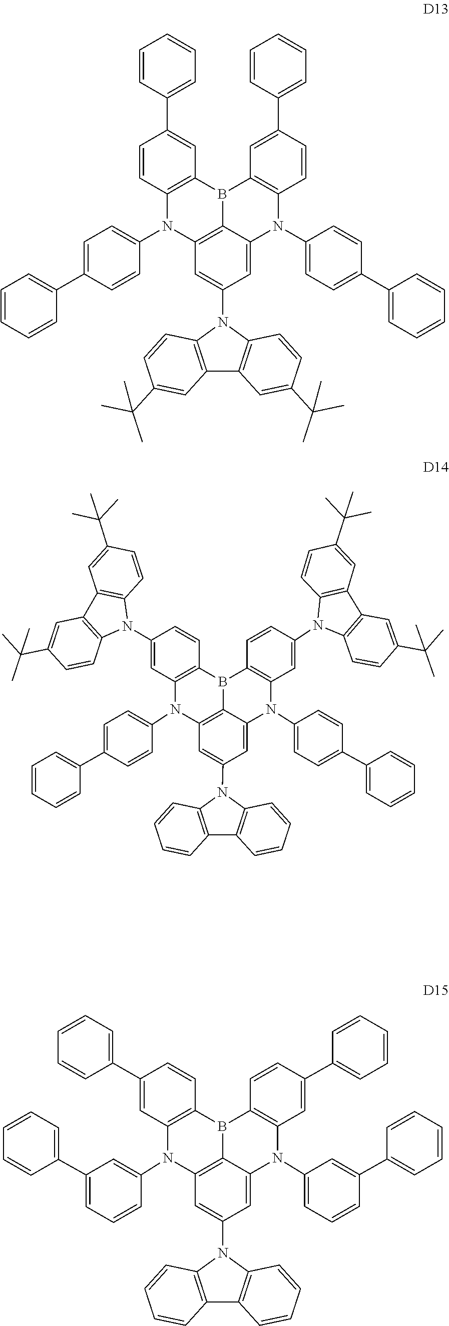 [SMILES: CC(C)(C)c1ccc2c(c1)c1cc(C(C)(C)C)ccc1n2-c1cc2c3c(c1)N(c1ccc(-c4ccccc4)cc1)c1ccc(-c4ccccc4)cc1B3c1cc(-c3ccccc3)ccc1N2c1ccc(-c2ccccc2)cc1 ; CC(C)(C)c1ccc2c(c1)c1cc(C(C)(C)C)ccc1n2-c1ccc2c(c1)N(c1ccc(-c3ccccc3)cc1)c1cc(-n3c4ccccc4c4ccccc43)cc3c1B2c1ccc(-n2c4ccc(C(C)(C)C)cc4c4cc(C(C)(C)C)ccc42)cc1N3c1ccc(-c2ccccc2)cc1 ; c1ccc(-c2cccc(N3c4cc(-c5ccccc5)ccc4B4c5ccc(-c6ccccc6)cc5N(c5cccc(-c6ccccc6)c5)c5cc(-n6c7ccccc7c7ccccc76)cc3c54)c2)cc1]

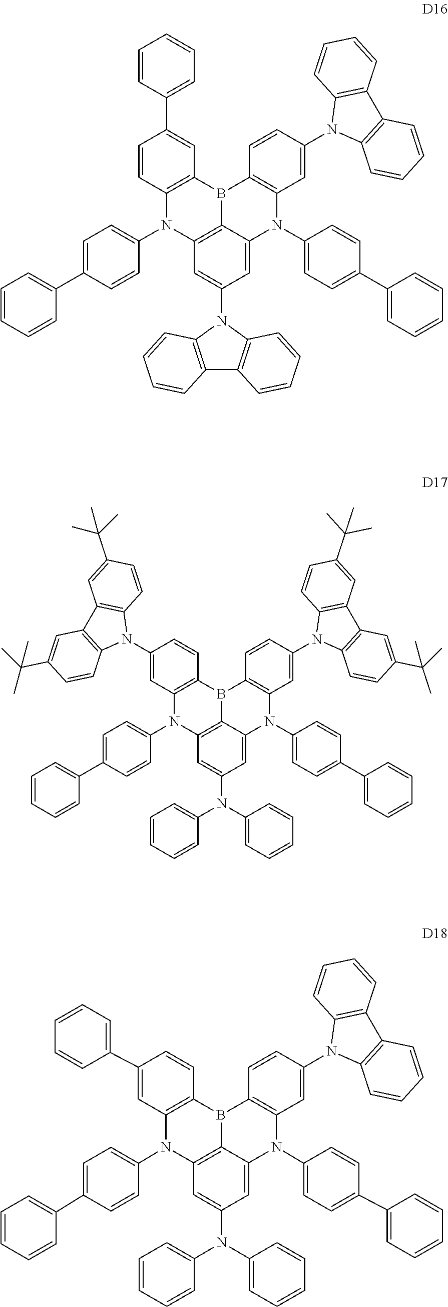 [SMILES: c1ccc(-c2ccc(N3c4ccc(-c5ccccc5)cc4B4c5ccc(-n6c7ccccc7c7ccccc76)cc5N(c5ccc(-c6ccccc6)cc5)c5cc(-n6c7ccccc7c7ccccc76)cc3c54)cc2)cc1 ; CC(C)(C)c1ccc2c(c1)c1cc(C(C)(C)C)ccc1n2-c1ccc2c(c1)N(c1ccc(-c3ccccc3)cc1)c1cc(N(c3ccccc3)c3ccccc3)cc3c1B2c1ccc(-n2c4ccc(C(C)(C)C)cc4c4cc(C(C)(C)C)ccc42)cc1N3c1ccc(-c2ccccc2)cc1 ; c1ccc(-c2ccc(N3c4cc(-c5ccccc5)ccc4B4c5ccc(-n6c7ccccc7c7ccccc76)cc5N(c5ccc(-c6ccccc6)cc5)c5cc(N(c6ccccc6)c6ccccc6)cc3c54)cc2)cc1]

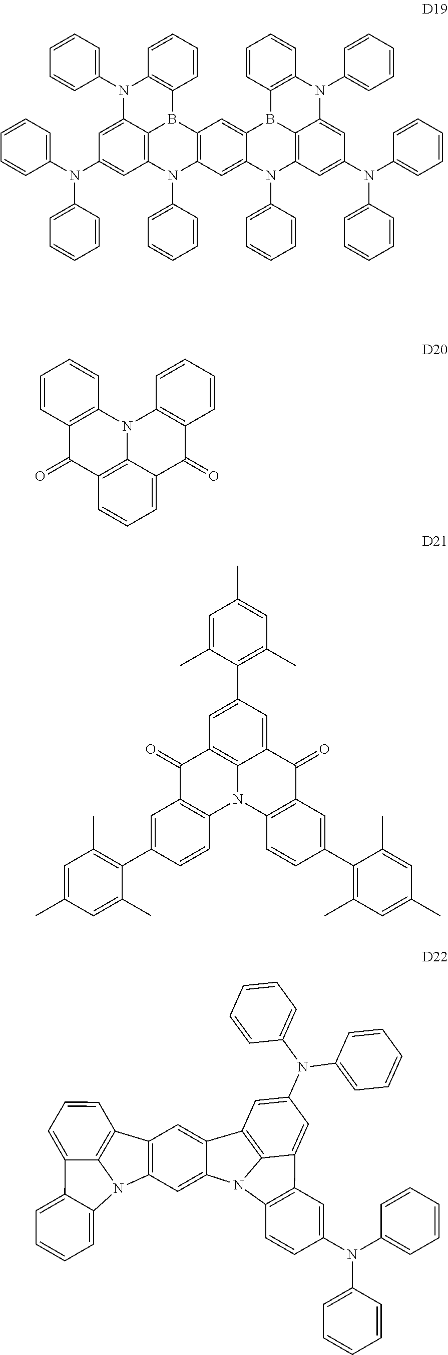

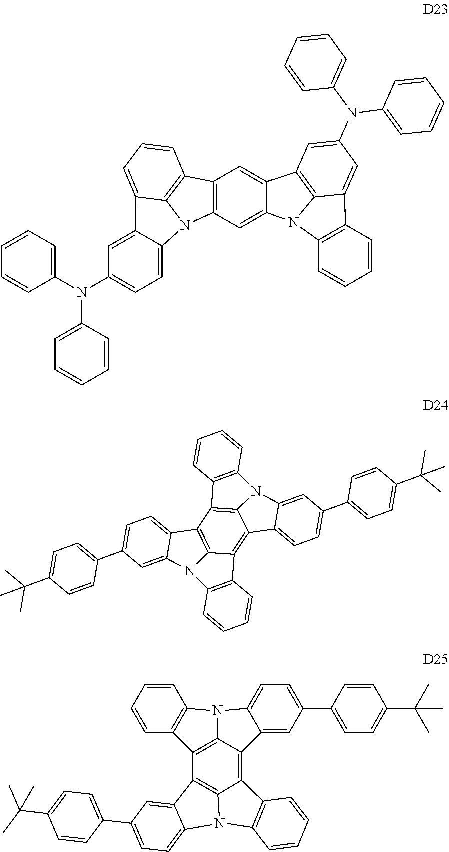

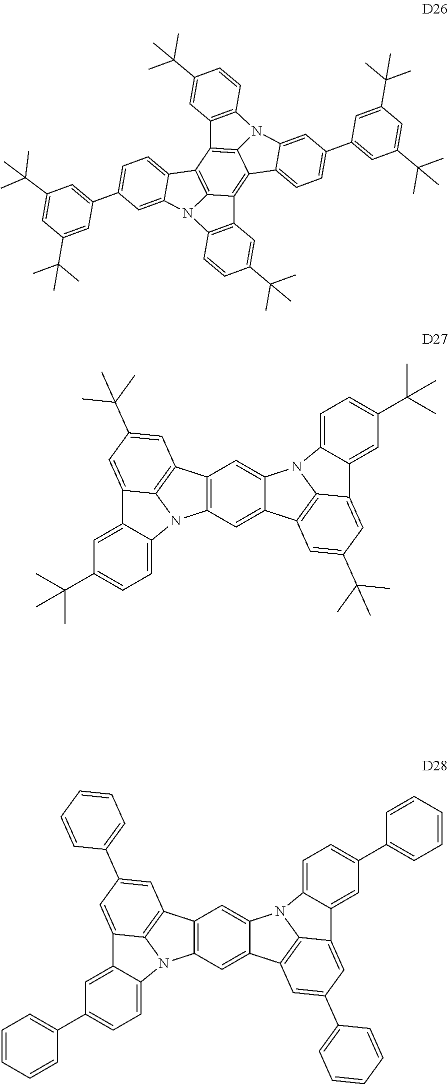

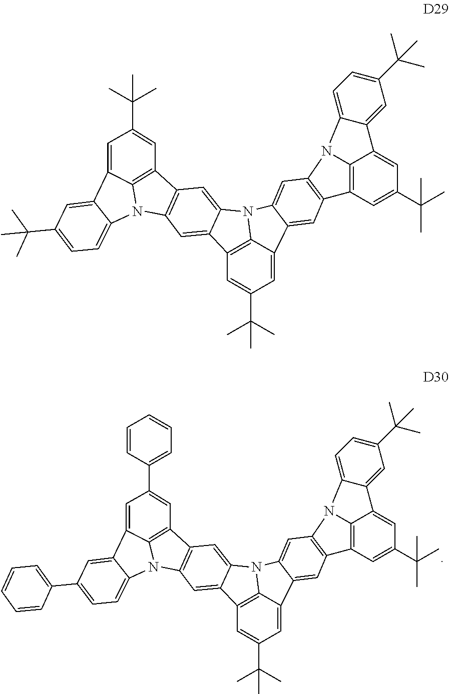 [SMILES: CC(C)(C)c1ccc2c(c1)c1cc(C(C)(C)C)cc3c4cc5c(cc4n2c13)c1cc(C(C)(C)C)cc2c3cc4c6cc(C(C)(C)C)cc7c8cc(C(C)(C)C)ccc8n(c4cc3n5c12)c76 ; CC(C)(C)c1ccc2c(c1)c1cc(C(C)(C)C)cc3c4cc5c6cc(C(C)(C)C)cc7c8cc9c(cc8n(c5cc4n2c13)c76)c1cc(-c2ccccc2)cc2c3cc(-c4ccccc4)ccc3n9c21]

In one or more embodiments, the fluorescent dopant may be included in the emission layer in an amount of about 0 wt % to about 5 wt %, based on total weight of the emission layer.

### Hole Transport Region

The hole transport region 12 may be arranged between the first electrode 11 and the emission layer 15 of the organic light-emitting device 10.

The hole transport region 12 may have a single-layer structure or a multi-layer structure.

For example, the hole transport region 12 may have a hole injection layer, a hole transport layer, a hole injection layer/hole transport layer structure, a hole injection layer/first hole transport layer/second hole transport layer structure, a hole injection layer/first hole transport layer/second hole transport layer/electron blocking layer structure, a hole transport layer/organic layer structure, a hole injection layer/hole transport layer/organic layer structure, a hole transport layer/electron blocking layer structure, or a hole injection layer/hole transport layer/electron blocking layer structure.

The hole transport region 12 may include any compound having hole-transporting properties.

The hole transport region 12 may include at least one of the heterocyclic compounds represented by Formula 1. For example, the heterocyclic compound may be included in a hole transport layer, a hole transport layer, an electron blocking layer, or a combination thereof of the hole transport region 12.

For example, the hole transport region 12 may include an amine-based compound.

In one or more embodiments, the hole transport region 12 may include 4,4′,4″-tris(3-methylphenylphenylamino)triphenylamine (m-MTDATA), 4,4′,4″-tris(N,N-diphenylamino)triphenylamine (TDATA), 4,4′,4″-tris{N-(2-naphthyl)-N-phenylamino}-triphenylamine (2-TNATA), N,N′-di(1-naphthyl)-N,N′-diphenylbenzidine (NPB), β-NPB, N,N bis(3-rethylphenyl)-N,N′-diphenyl-[1,1-biphenyl]-4,4′-diamine (TPD), spiro-TPD, spiro-NPB, methylated-NPB, 4,4′-cyclohexylidene bis[N,N-bis(4-methylphenyl)benzenamine] (TAPC), HMTPD, 4,4′,4″-tris(N-carbazolyl)triphenylamine (TCTA), polyaniline/dodecylbenzenesulfonic acid (PANI/DBSA), poly(3,4-ethylenedioxythiophene)/poly(4-styrenesulfonate) (PEDOT/PSS), polyaniline/camphor-sulfonic acid (PANI/CSA), polyaniline/poly(4-styrene sulfonate) (PANI/PSS), a compound represented by one of Formulae 201 to 205, or a combination thereof, but embodiments are not limited thereto:

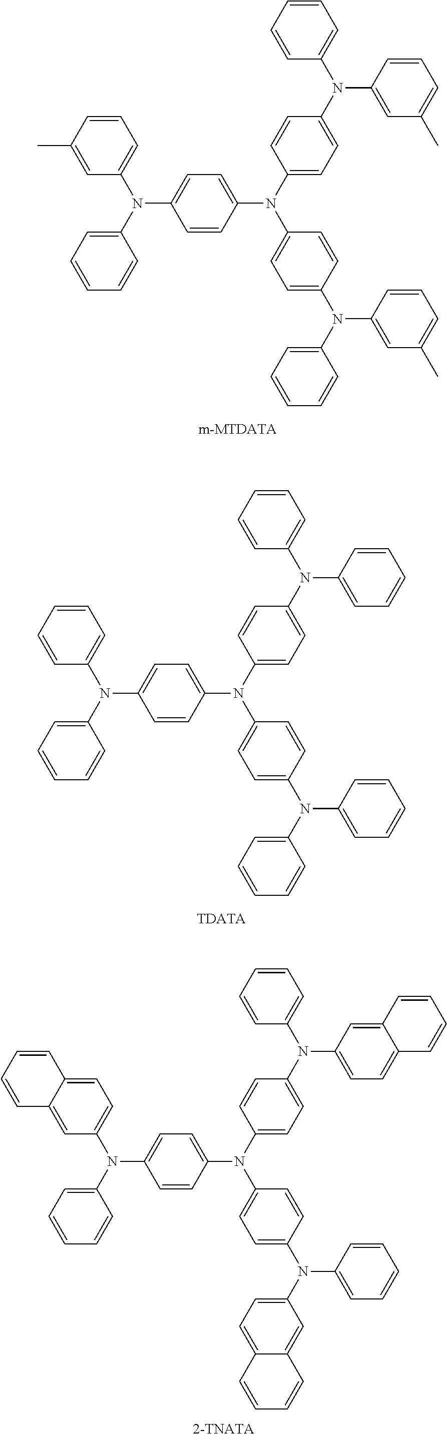

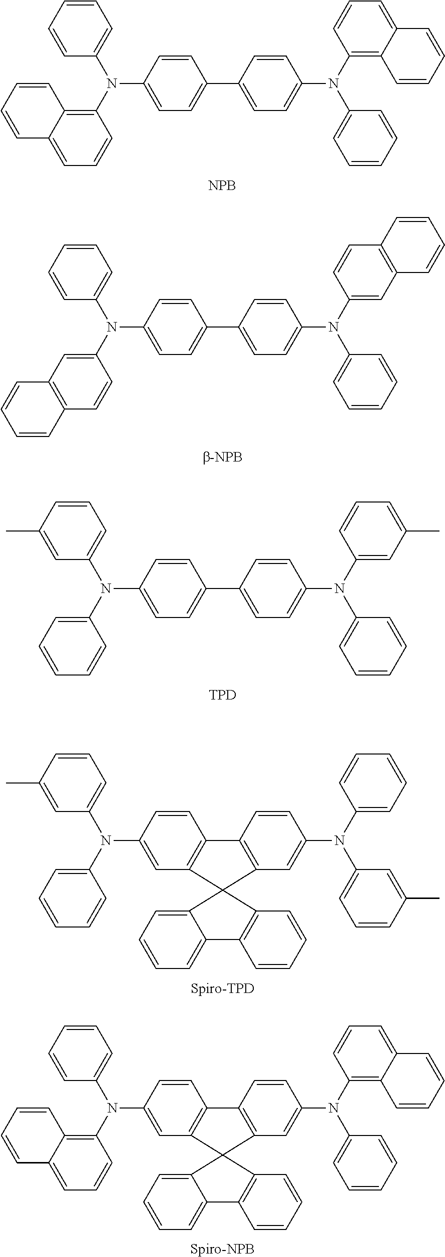

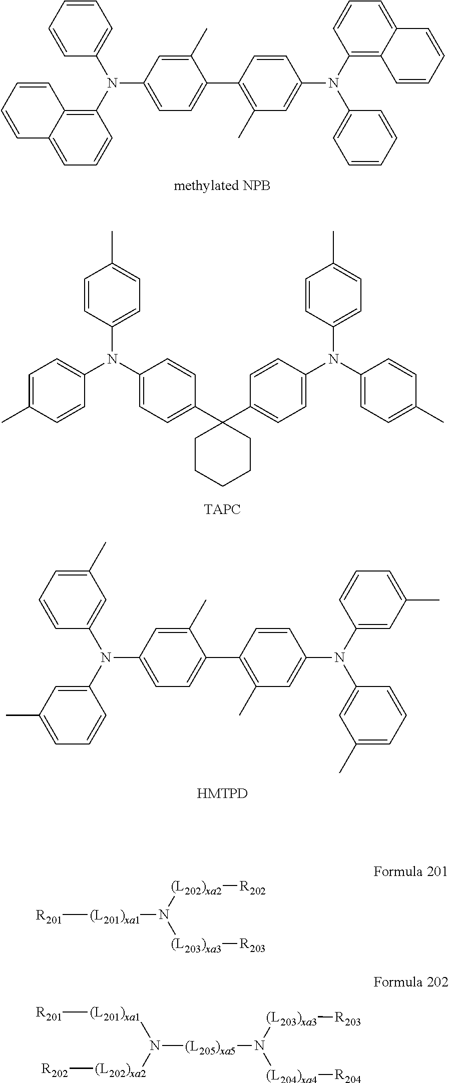

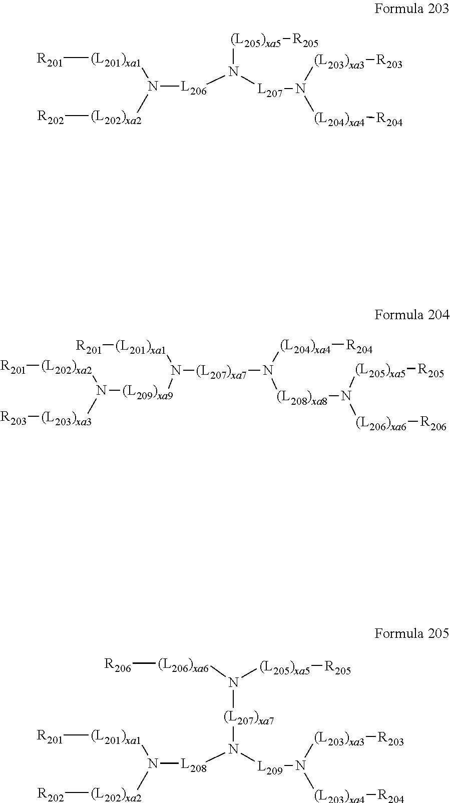

wherein, in Formulae 201 to 205,
L₂₀₁ to L₂₀₉ may each independently be *—O—*′, *—S—*′, a substituted or unsubstituted C₅-C₆₀ carbocyclic group, or a substituted or unsubstituted C₁-C₆₀ heterocyclic group,
xa1 to xa9 may each independently be an integer from 0 to 5, and
R₂₀₁ to R₂₀₆ may each independently be a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₇-C₆₀ alkyl aryl group, a substituted or unsubstituted C₇-C₆₀ aryl alkyl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted C₂-C₆₀ alkyl heteroaryl group, a substituted or unsubstituted C₂-C₆₀ heteroaryl alkyl group, a substituted or unsubstituted C₁-C₆ heteroaryloxy group, a substituted or unsubstituted C₁-C₆₀ heteroarylthio group, a substituted or unsubstituted monovalent non-aromatic condensed polycyclic group, or a substituted or unsubstituted monovalent non-aromatic condensed heteropolycyclic group, wherein neighboring two groups of R₂₀₁ to R₂₀₆ may optionally be linked to each other via a single bond, a dimethyl-methylene group, or a diphenyl-methylene group.

For example,
L₂₀₁ to L₂₀₉ may be a benzene group, a heptalene group, an indene group, a naphthalene group, an azulene group, a heptalene group, an indacene group, an acenaphthylene group, a fluorene group, a spiro-bifluorene group, a benzofluorene group, a dibenzofluorene group, a phenalene group, a phenanthrene group, an anthracene group, a fluoranthene group, a triphenylene group, a pyrene group, a chrysene group, a naphthacene group, a picene group, a perylene group, a pentacene group, a hexacene group, a pentacene group, a rubicene group, a coronene group, an ovalene group, a pyrrole group, an isoindole group, an indole group, a furan group, a thiophene group, a benzofuran group, a benzothiophene group, a benzocarbazole group, a dibenzocarbazole group, a dibenzofuran group, a dibenzothiophene group, a dibenzothiophene sulfone group, a carbazole group, a dibenzosilole group, an indenocarbazole group, an indolocarbazole group, a benzofurocarbazole group, a benzothienocarbazole group, or a triindolobenzene group, each unsubstituted or substituted with at least one of deuterium, a C₁-C₁₀ alkyl group, a C₁-C₁₀ alkoxy group, a C₁-C₆₀ alkylthio group, a phenyl group, a naphthyl group, a fluorenyl group, a carbazolyl group, a dibenzofuranyl group, a dibenzothiophenyl group, a triphenylenyl group, a biphenyl group, a terphenyl group, a tetraphenyl group, —Si(Q₁₁)(Q₁₂)(Q₁₃), or a combination thereof,
xa1 to xa9 may each independently be 0, 1, or 2, and

R₂₀₁ to R₂₀₆ may each independently be a phenyl group, a biphenyl group, a terphenyl group, a pentalenyl group, an indenyl group, a naphthyl group, an azulenyl group, a heptalenyl group, an indacenyl group, an acenaphthyl group, a fluorenyl group, a spiro-bifluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, an ovalenyl group, a thiophenyl group, a furanyl group, a carbazolyl group, an indolyl group, an isoindolyl group, a benzofuranyl group, a benzothiophenyl group, a dibenzofuranyl group, a dibenzothiophenyl group, a benzocarbazolyl group, a dibenzocarbazolyl group, a dibenzosilolyl group, a pyridinyl group, an indeno carbazolyl group, an indolocarbazolyl group, a benzofurocarbazolyl group, or a benzothienocarbazolyl group, each unsubstituted or substituted with at least one of deuterium, —F, —Cl, —Br, —I, —SF₅, a hydroxyl group, a cyano group, a nitro group, an amino group, an amidino group, a hydrazine group, a hydrazone group, a C₁-C₂₀ alkyl group, a C₁-C₂₀ alkoxy group, a C₁-C₂₀ alkylthio group, a cyclopentyl group, a cyclohexyl group, a cycloheptyl group, a cyclopentenyl group, a cyclohexenyl group, a phenyl group, a biphenyl group, a terphenyl group, a phenyl group substituted with a C₁-C₁₀ alkyl group, a phenyl group substituted with —F, a pentalenyl group, an indenyl group, a naphthyl group, an azulenyl group, a heptalenyl group, an indacenyl group, an acenaphthyl group, a fluorenyl group, a spiro-bifluorenyl group, a benzofluorenyl group, a dibenzofluorenyl group, a phenalenyl group, a phenanthrenyl group, an anthracenyl group, a fluoranthenyl group, a triphenylenyl group, a pyrenyl group, a chrysenyl group, a naphthacenyl group, a picenyl group, a perylenyl group, a pentaphenyl group, a hexacenyl group, a pentacenyl group, a rubicenyl group, a coronenyl group, an ovalenyl group, a thiophenyl group, a furanyl group, a carbazolyl group, an indolyl group, an isoindolyl group, a benzofuranyl group, a benzothiophenyl group, a dibenzofuranyl group, a dibenzothiophenyl group, a benzocarbazolyl group, a dibenzocarbazolyl group, a dibenzosilolyl group, a pyridinyl group, —Si(Q₃₁)(Q₃₂)(Q₃₃), —N(Q₃₁)(Q₃₂), or a combination thereof.

Q₁₁ to Q₁₃ and Q₃₁ to Q₃₃ may each independently be a C₁-C₁₀ alkyl group, a C₁-C₁₀ alkoxy group, a C₁-C₁₀ alkylthio group, a phenyl group, a biphenyl group, a terphenyl group, or a naphthyl group.

In one or more embodiments, the hole transport region 12 may include a carbazole-containing amine compound.

In one or more embodiments, the hole transport region 12 may include a carbazole-containing amine compound and a carbazole-free amine compound.

The carbazole-containing amine compound may include, for example, one or more compounds represented by Formula 201 including a carbazole group and further including at least one of a dibenzofuran group, a dibenzothiophene group, a fluorene group, a spiro-bifluorene group, an indenocarbazole group, an indolocarbazole group, a benzofurocarbazole group, or a benzothienocarbazole group.

The carbazole-free amine compound may include, for example, one or more compounds represented by Formula 201 not including a carbazole group and including at least one of a dibenzofuran group, a dibenzothiophene group, a fluorene group, a spiro-bifluorene group, an indenocarbazole group, an indolocarbazole group, a benzofurocarbazole group, or a benzothienocarbazole group.

In one or more embodiments, the hole transport region 12 may include a compound represented by Formula 201, a compound represented by Formula 202, or a combination thereof.

In one or more embodiments, the hole transport region 12 may include a compound represented by Formula 201-1, 202-1, or 201-2, or a combination thereof:

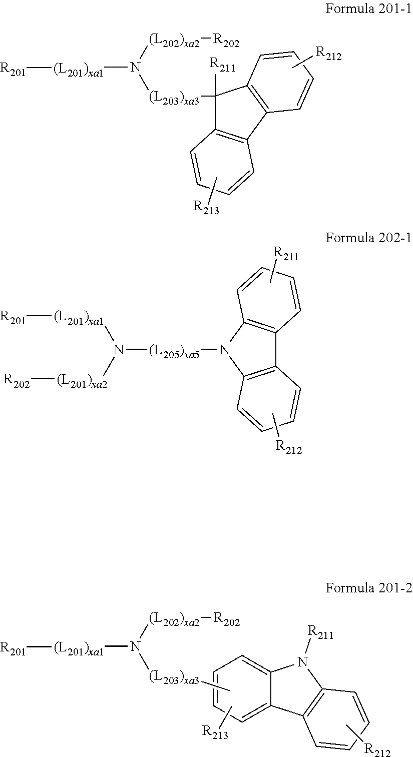

wherein, in Formulae 201-1, 202-1, and 201-2,
L₂₀₁ to L₂₀₃, L₂₀₅, xa1 to xa3, xa5, R₂₀₁ and R₂₀₂ may each be as described herein, and
R₂₁₁ to R₂₁₃ may each independently be hydrogen, deuterium, —F, —Cl, —Br, —I, —SF₅, a hydroxyl group, a cyano group, a nitro group, an amino group, an amidino group, a hydrazine group, a hydrazone group, a C₁-C₂₀ alkyl group, a C₁-C₂₀ alkoxy group, a C₁-C₂₀ alkylthio group, a phenyl group, a biphenyl group, a terphenyl group, a phenyl group substituted with a C₁-C₁₀ alkyl group, a phenyl group substituted with —F, a naphthyl group, a fluorenyl group, a spiro-bifluorenyl group, a dimethylfluorenyl group, a diphenylfluorenyl group, a triphenylenyl group, a thiophenyl group, a furanyl group, a carbazolyl group, an indolyl group, an isoindolyl group, a benzofuranyl group, a benzothiophenyl group, a dibenzofuranyl group, a dibenzothiophenyl group, a benzocarbazolyl group, a dibenzocarbazolyl group, a dibenzosilolyl group, or a pyridinyl group.

In one or more embodiments, the hole transport region 12 may include one of Compounds HT1 to HT39, or a combination thereof, but embodiments are not limited thereto:

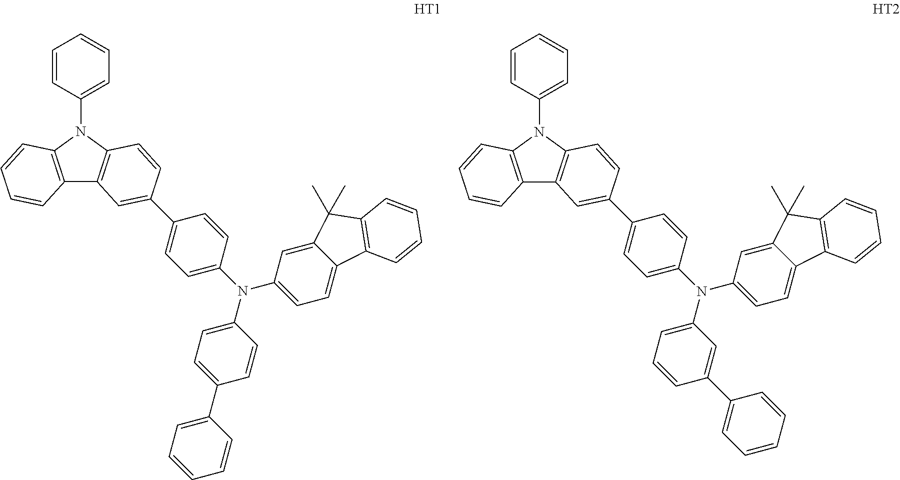 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4ccccc4)c3)cc21]

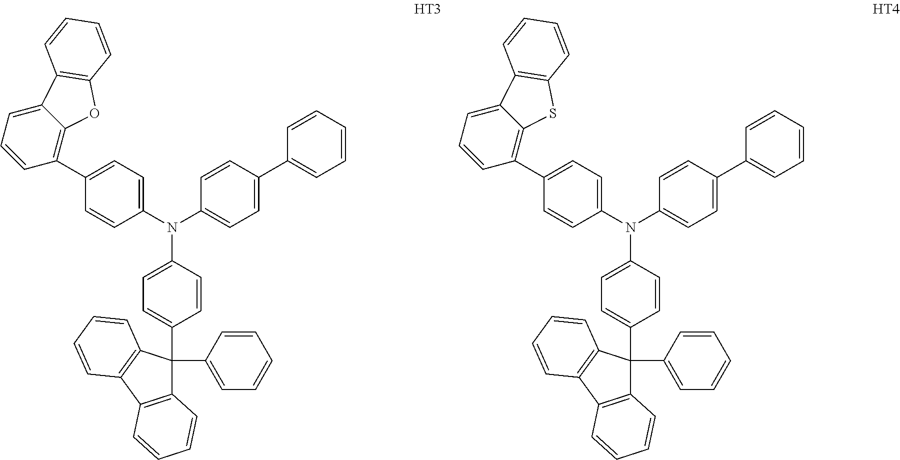 [SMILES: c1ccc(-c2ccc(N(c3ccc(-c4cccc5c4oc4ccccc45)cc3)c3ccc(C4(c5ccccc5)c5ccccc5-c5ccccc54)cc3)cc2)cc1 ; c1ccc(-c2ccc(N(c3ccc(-c4cccc5c4sc4ccccc45)cc3)c3ccc(C4(c5ccccc5)c5ccccc5-c5ccccc54)cc3)cc2)cc1]

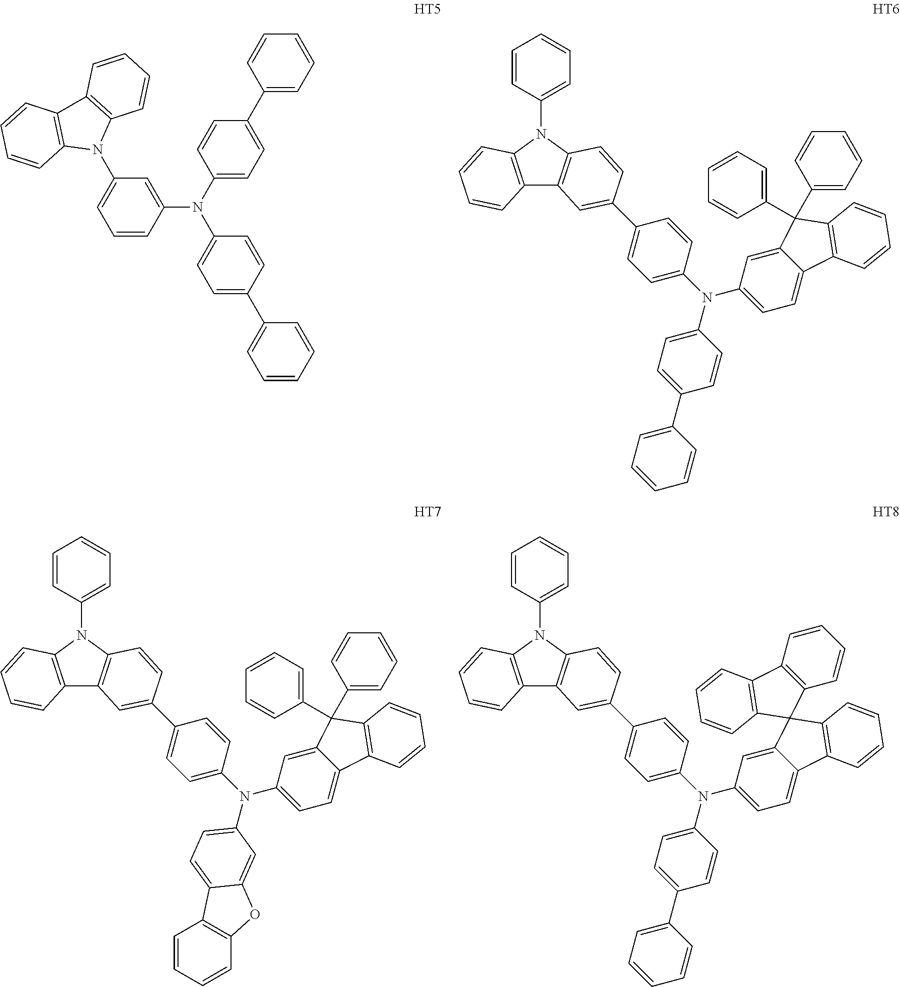 [SMILES: c1ccc(-c2ccc(N(c3ccc(-c4ccccc4)cc3)c3cccc(-n4c5ccccc5c5ccccc54)c3)cc2)cc1 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1 ; c1ccc(-n2c3ccccc3c3cc(-c4ccc(N(c5ccc6c(c5)C(c5ccccc5)(c5ccccc5)c5ccccc5-6)c5ccc6c(c5)oc5ccccc56)cc4)ccc32)cc1 ; c1ccc(-c2ccc(N(c3ccc(-c4ccc5c(c4)c4ccccc4n5-c4ccccc4)cc3)c3ccc4c(c3)C3(c5ccccc5-c5ccccc53)c3ccccc3-4)cc2)cc1]

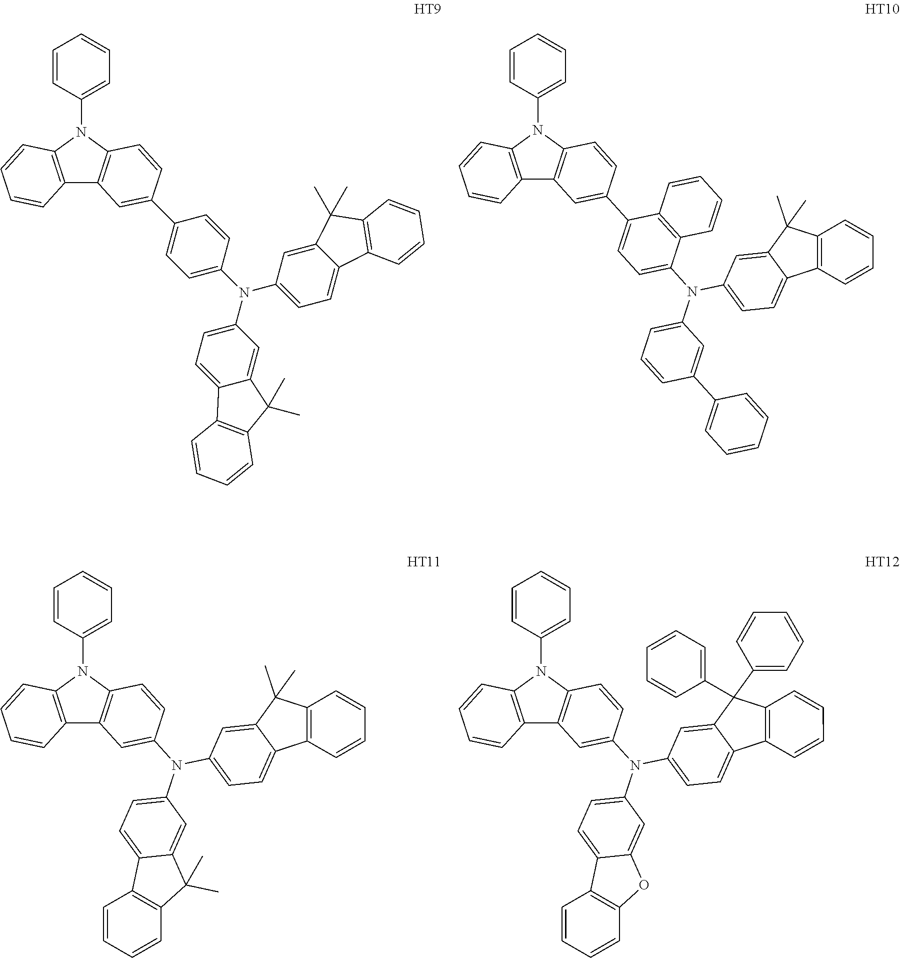 [SMILES: CC1(C)c2ccccc2-c2ccc(N(c3ccc(-c4ccc5c(c4)c4ccccc4n5-c4ccccc4)cc3)c3ccc4c(c3)C(C)(C)c3ccccc3-4)cc21 ; CC1(C)c2ccccc2-c2ccc(N(c3cccc(-c4ccccc4)c3)c3ccc(-c4ccc5c(c4)c4ccccc4n5-c4ccccc4)c4ccccc34)cc21 ; CC1(C)c2ccccc2-c2ccc(N(c3ccc4c(c3)C(C)(C)c3ccccc3-4)c3ccc4c(c3)c3ccccc3n4-c3ccccc3)cc21 ; c1ccc(-n2c3ccccc3c3cc(N(c4ccc5c(c4)C(c4ccccc4)(c4ccccc4)c4ccccc4-5)c4ccc5c(c4)oc4ccccc45)ccc32)cc1]

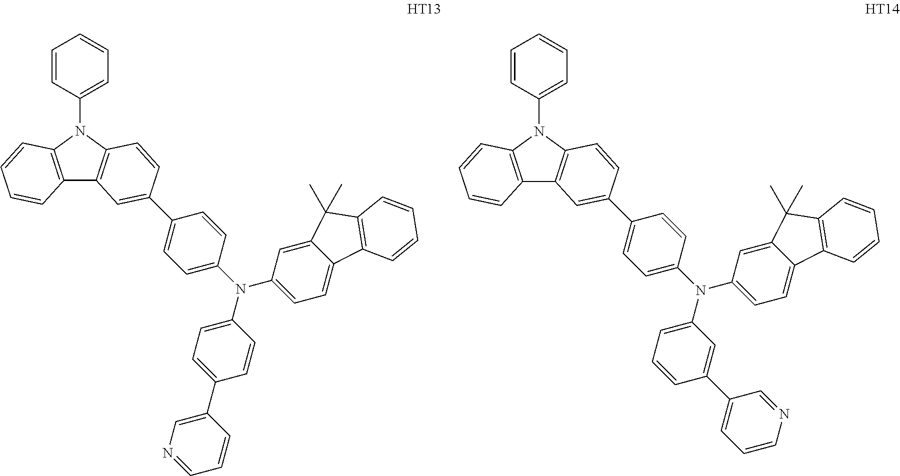 [SMILES: CC1(C)c2ccccc2-c2ccc(N(c3ccc(-c4cccnc4)cc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4cccnc4)c3)cc21]

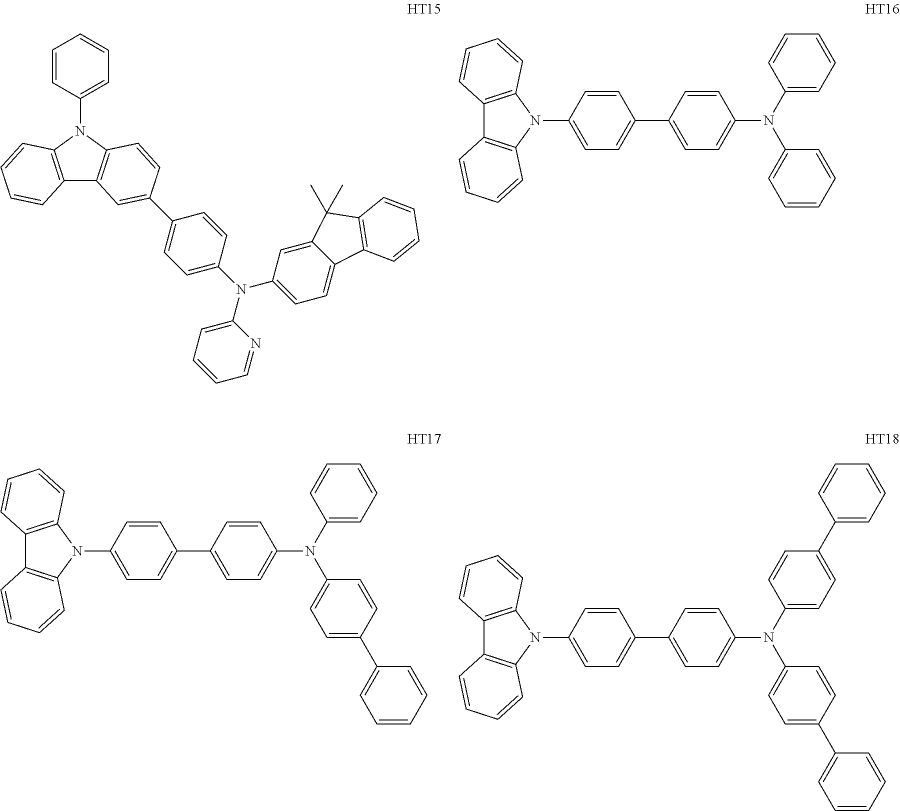

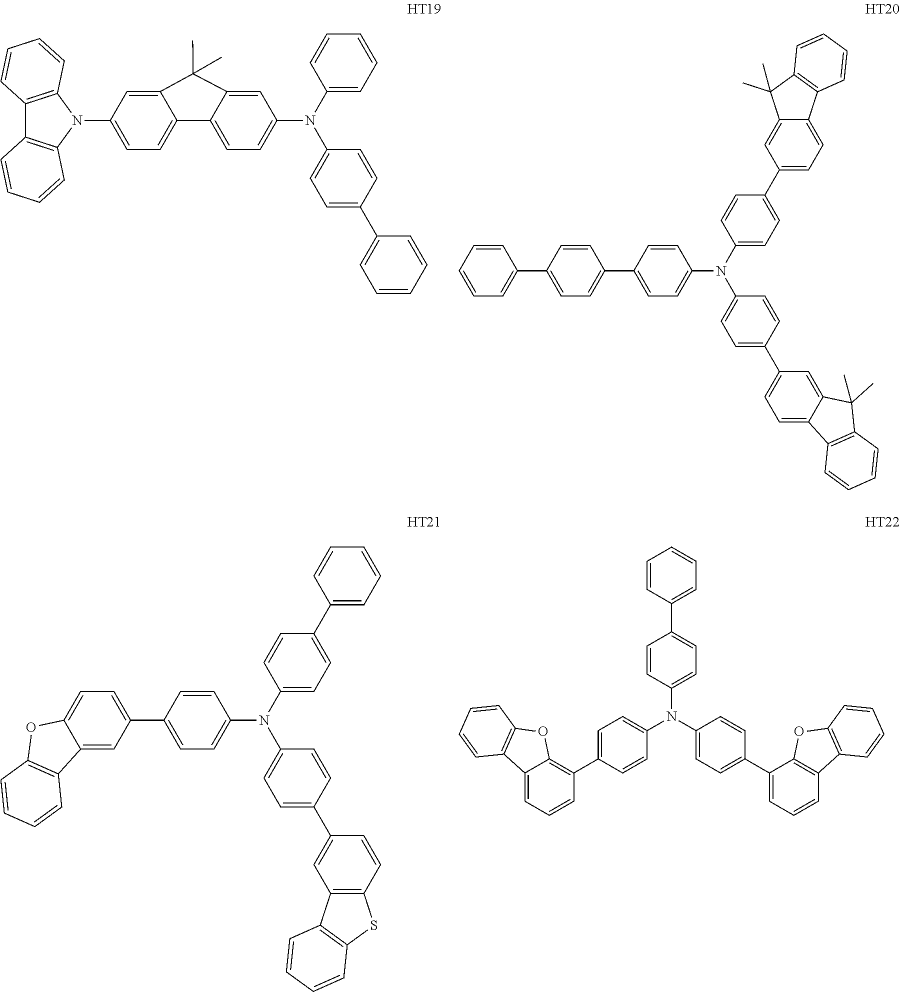

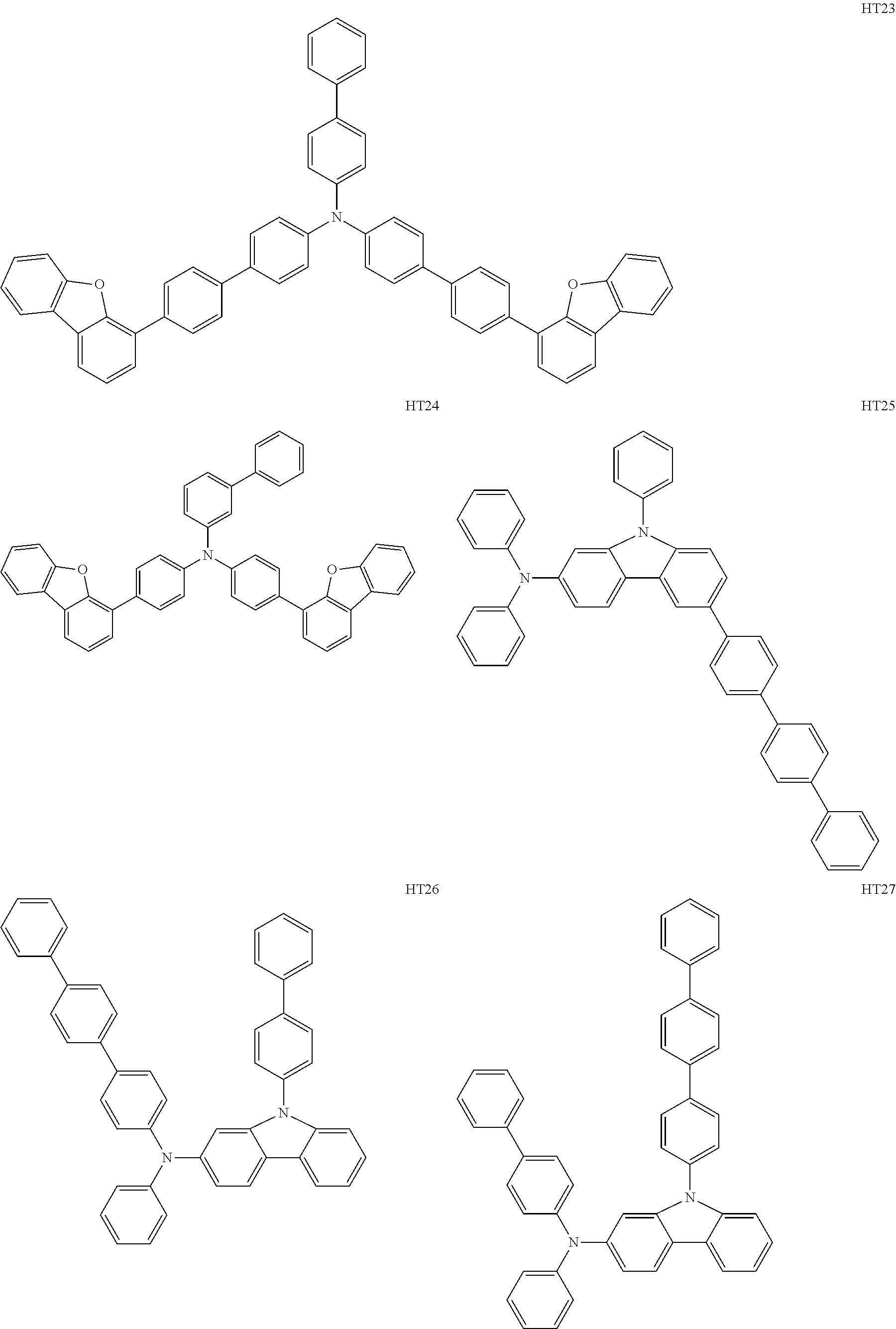 [SMILES: c1ccc(-c2ccc(N(c3ccc(-c4ccc(-c5cccc6c5oc5ccccc56)cc4)cc3)c3ccc(-c4ccc(-c5cccc6c5oc5ccccc56)cc4)cc3)cc2)cc1 ; c1ccc(-c2cccc(N(c3ccc(-c4cccc5c4oc4ccccc45)cc3)c3ccc(-c4cccc5c4oc4ccccc45)cc3)c2)cc1 ; c1ccc(-c2ccc(-c3ccc(-c4ccc5c(c4)c4ccc(N(c6ccccc6)c6ccccc6)cc4n5-c4ccccc4)cc3)cc2)cc1 ; c1ccc(-c2ccc(-c3ccc(N(c4ccccc4)c4ccc5c6ccccc6n(-c6ccc(-c7ccccc7)cc6)c5c4)cc3)cc2)cc1 ; c1ccc(-c2ccc(-c3ccc(-n4c5ccccc5c5ccc(N(c6ccccc6)c6ccc(-c7ccccc7)cc6)cc54)cc3)cc2)cc1]

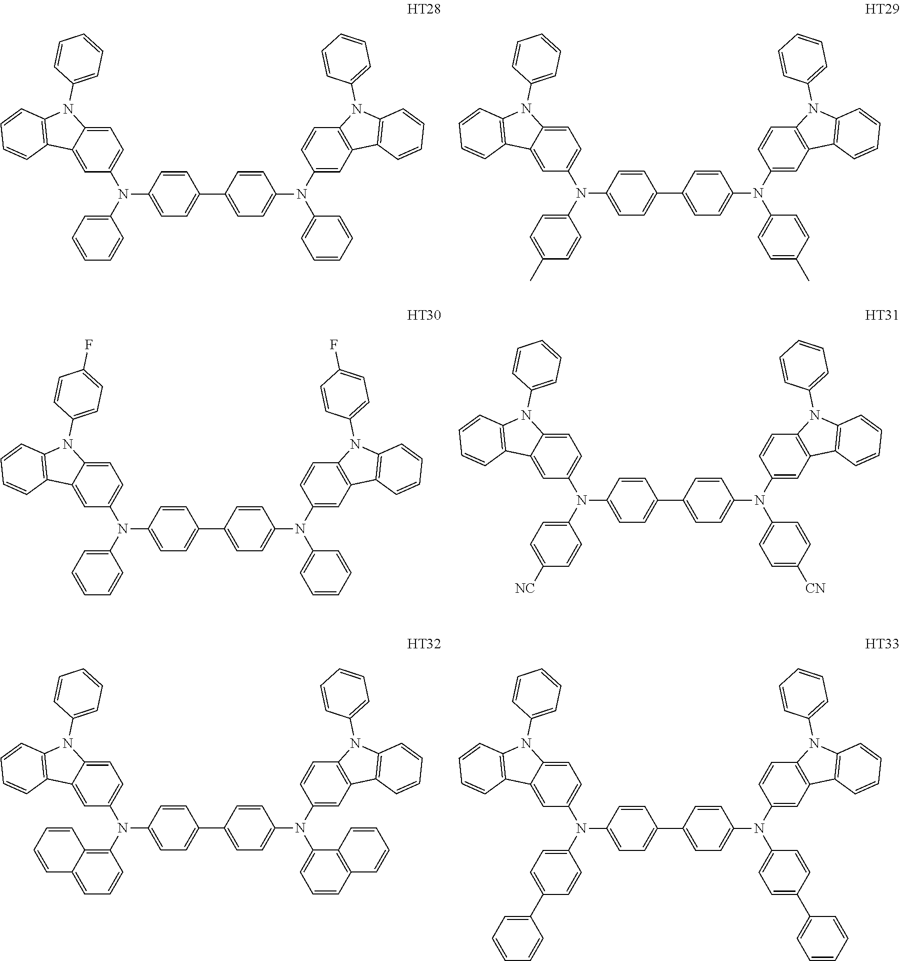

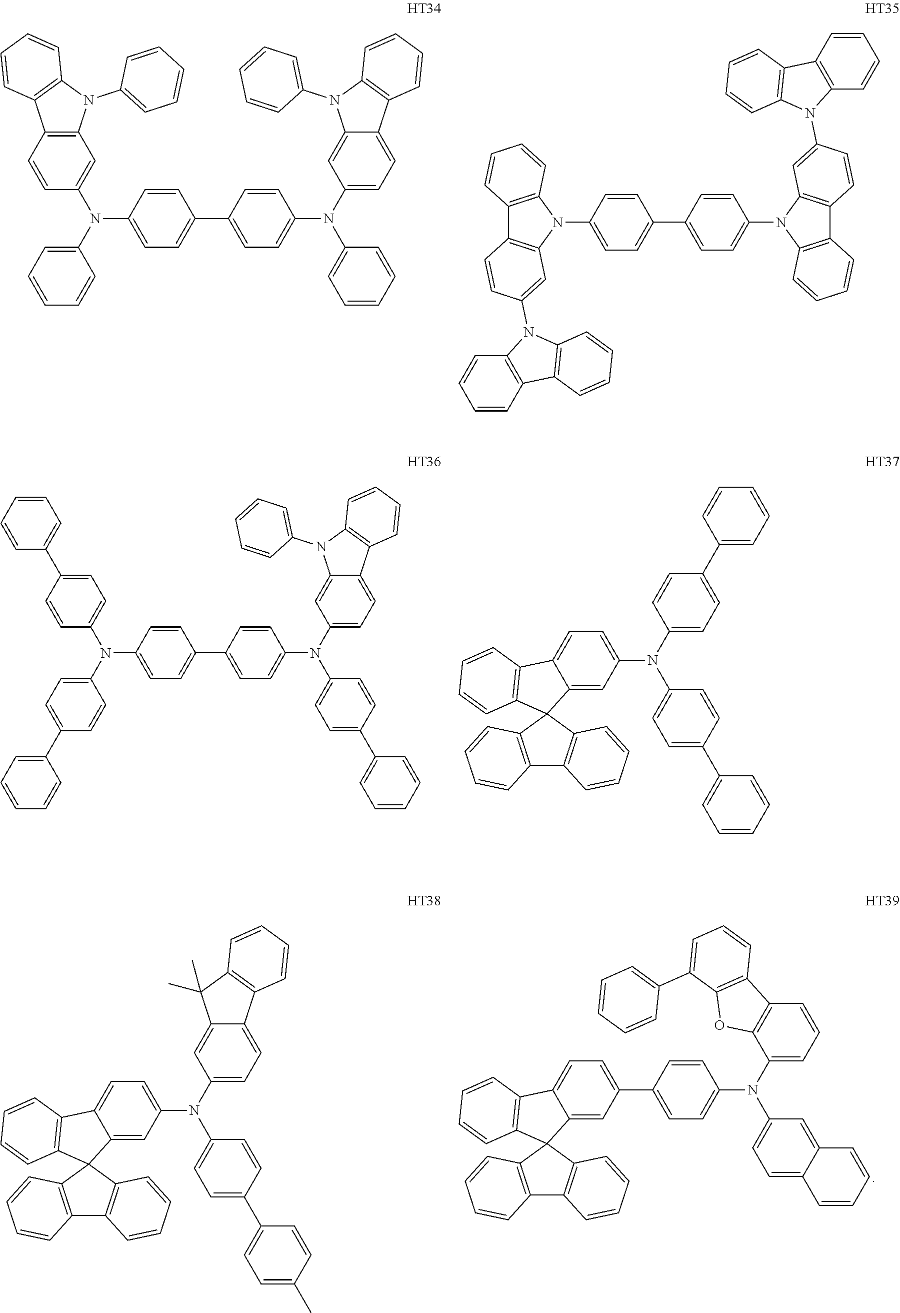

In one or more embodiments, the hole transport region 12 of the organic light-emitting device 10 may further include a p-dopant. When the hole transport region 12 further includes a p-dopant, the hole transport region 12 may have a matrix (for example, at least one of compounds represented by Formulae 201 to 205) and a p-dopant included in the matrix. The p-dopant may be uniformly or non-uniformly doped in the hole transport region 12.

In one or more embodiments, the LUMO energy level of the p-dopant may be less than or equal to about −3.5 eV.

The p-dopant may include a quinone derivative, a metal oxide, a cyano group-containing compound, or a combination thereof.

For example, the p-dopant may include:
a quinone derivative, such as tetracyanoquinodimethane (TCNQ), 2,3,5,6-tetrafluoro-7,7,8,8-tetracyanoquinodimethane (F4-TCNQ), 1,3,4,5,7,8-hexafluorotetracyanonaphthoquinodimethane (F6-TCNQ), or the like;
a metal oxide, such as a tungsten oxide, a molybdenum oxide, or the like;
1,4,5,8,9,12-hexaazatriphenylene-hexacarbonitrile (HAT-CN);
a compound represented by Formula 221; or
a combination thereof, but embodiments are not limited thereto:

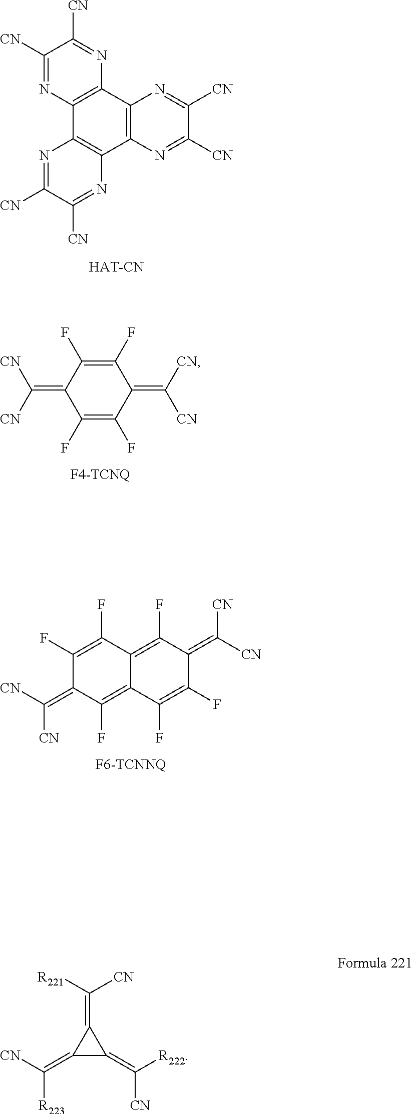

wherein, in Formula 221,
R₂₂₁ to R₂₂₃ may each independently be a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₁-C₆₀ heteroaryl group, a substituted or unsubstituted monovalent non-aromatic condensed polycyclic group, or a substituted or unsubstituted monovalent non-aromatic condensed heteropolycyclic group, wherein at least one substituent of R₂₂₁ to R₂₂₃ may be:
a cyano group, —F, —Cl, —Br, —I, a C₁-C₂₀ alkyl group substituted with —F, a C₁-C₂₀ alkyl group substituted with —Cl, a C₁-C₂₀ alkyl group substituted with —Br, a C₁-C₂₀ alkyl group substituted with —I, or a combination thereof.

The compound represented by Formula 221 may include, for example, Compound HT-D2:

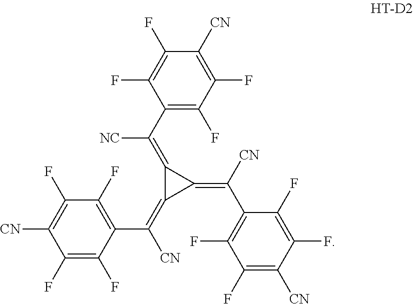

The hole transport region 12 may have a thickness of about 100 Å to about 10,000 Å, for example, about 400 Å to about 2,000 Å, and the emission layer 15 may have a thickness of about 100 Å to about 3,000 Å, for example, about 300 Å to about 1,000 Å. Without wishing to be bound to theory, when the thickness of each of the hole transport region 12 and the emission layer 15 is within these ranges, satisfactory hole transportation characteristics and/or luminescence characteristics may be obtained without a substantial increase in driving voltage.

The hole transport region 12 may further include a buffer layer.

The buffer layer may compensate for an optical resonance distance according to a wavelength of light emitted from the emission layer 15, and thus, efficiency of a formed organic light-emitting device may be further improved.

The hole transport region 12 may further include an electron blocking layer. The electron blocking layer may include a known material, for example, mCP or 2,8-bis(diphenylphosphineoxide)dibenzofuran (DBFPO), but embodiments are not limited thereto:

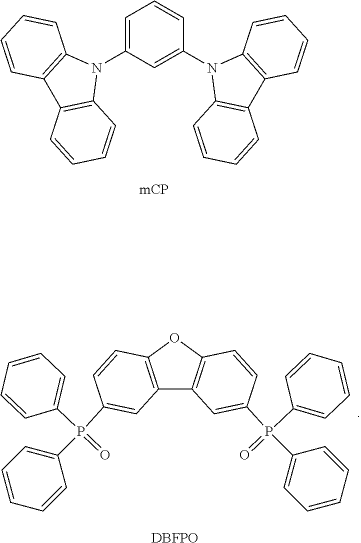

### Electron Transport Region

The electron transport region 17 may be arranged between the emission layer 15 and the second electrode 19 of the organic light-emitting device 10.

The electron transport region 17 may have a single-layer structure or a multi-layer structure.

For example, the electron transport region 17 may have an electron transport layer, an electron transport layer/electron injection layer structure, a buffer layer/electron transport layer structure, hole blocking layer/electron transport layer structure, a buffer layer/electron transport layer/electron injection layer structure, or a hole blocking layer/electron transport layer/electron injection layer structure. The electron transport region 17 may further include an electron control layer.

The electron transport region 17 may include at least one of the heterocyclic compounds represented by Formula 1. For example, the heterocyclic compound may be included in a buffer layer or the like of the electron transport region 17.

The electron transport region 17 may include a known electron-transporting materials.

The electron transport region 17 (for example, a buffer layer, a hole blocking layer, an electron control layer, or an electron transport layer in the electron transport region) may include a metal-free compound containing at least one π electron-deficient nitrogen-containing C₁-C₆₀ cyclic group. The π electron-deficient nitrogen-containing C₁-C₆₀ cyclic group may be as described herein.

For example, the electron transport region 17 may include a compound represented by Formula 601, but embodiments are not limited thereto:

[Ar₆₀₁]ₓₑ₁₁-[(L₆₀₁)ₓₑ₁-R₆₀₁]ₓₑ₂₁ Formula 601

wherein, in Formula 601,
Ar₆₀₁ and L₆₀₁ may each independently be a C₅-C₆₀ carbocyclic group unsubstituted or substituted with at least one R₆₀₁ₐ, or a C₁-C₆₀ heterocyclic group unsubstituted or substituted with at least one R₆₀₁ₐ,
xe11 may be 1, 2, or 3,
xe1 may be an integer from 0 to 5,
R₆₀₁ₐ and R₆₀₁ may each independently be a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₇-C₆₀ alkyl aryl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted C₂-C₆₀ alkyl heteroaryl group, a substituted or unsubstituted C₁-C₆₀ heteroaryloxy group, a substituted or unsubstituted C₁-C₆₀ heteroarylthio group, a substituted or unsubstituted monovalent non-aromatic condensed polycyclic group, a substituted or unsubstituted monovalent non-aromatic condensed heteropolycyclic group, —Si(Q₆₀₁)(Q₆₀₂)(Q₆₀₃), —N(Q₆₀₁)(Q₆₀₂), —C(═O)(Q₆₀₁), —S(═O)₂(Q₆₀₁), —P(═O)(Q₆₀₁)(Q₆₀₂), or —P(Q₆₀₁)(Q₆₀₂),
Q₆₀₁ to Q₆₀₃ may each independently be a C₁-C₁₀ alkyl group, a C₁-C₁₀ alkoxy group, a C₁-C₁₀ alkylthio group, a phenyl group, a biphenyl group, a terphenyl group, or a naphthyl group, and
xe21 may be an integer from 1 to 5.

In one or more embodiments, at least one of Ar₆₀₁ and R₆₀₁ may include the π electron-deficient nitrogen-containing C₁-C₆₀ cyclic group.

In one or more embodiments, Ar₆₀₁ and L₆₀₁ in Formula 601 may each independently be a benzene group, a naphthalene group, a fluorene group, a spiro-bifluorene group, a benzofluorene group, a dibenzofluorene group, a phenalene group, a phenanthrene group, an anthracene group, a fluoranthene group, a triphenylene group, a pyrene group, a chrysene group, a naphthacene group, a picene group, a perylene group, a pentaphene group, an indenoanthracene group, a dibenzofuran group, a dibenzothiophene group, a carbazole group, an imidazole group, a pyrazole group, a thiazole group, an isothiazole group, an oxazole group, an isoxazole group, a pyridine group, a pyrazine group, a pyrimidine group, a pyridazine group, an indazole group, a purine group, a quinoline group, an isoquinoline group, a benzoquinoline group, a phthalazine group, a naphthyridine group, a quinoxaline group, a quinazoline group, a cinnoline group, a phenanthridine group, an acridine group, a phenanthroline group, a phenazine group, a benzimidazole group, an isobenzothiazole group, a benzoxazole group, an isobenzoxazole group, a triazole group, a tetrazole group, an oxadiazole group, a triazine group, a thiadiazole group, an imidazopyridine group, an imidazopyrimidine group, or an azacarbazole group, each unsubstituted or substituted with at least one of deuterium, —F, —Cl, —Br, —I, —SF₅, a hydroxyl group, a cyano group, a nitro group, an amino group, an amidino group, a hydrazino group, a hydrazono group, a C₁-C₂₀ alkyl group, a C₁-C₂₀ alkoxy group, a C₁-C₂₀ alkylthio group, a phenyl group, a biphenyl group, a terphenyl group, a naphthyl group, —Si(Q₃₁)(Q₃₂)(Q₃₃), —S(═O)₂(Q₃₁), —P(═O)(Q₃₁)(Q₃₂), —P(Q₃₁)(Q₃₂), or a combination thereof, and
Q₃₁ to Q₃₃ may each independently be a C₁-C₁₀ alkyl group, a C₁-C₁₀ alkoxy group, a C₁-C₁₀ alkylthio group, a phenyl group, a biphenyl group, a terphenyl group, or a naphthyl group.

When xe11 in Formula 601 is 2 or more, two or more of Ar₆₀₁ may be linked to each other via a single bond.

In one or more embodiments, Ar₆₀₁ in Formula 601 may be an anthracene group.

In one or more embodiments, the compound represented by Formula 601 may be represented by Formula 601-1:

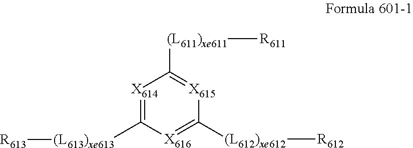

wherein, in Formula 601-1,
X₆₁₄ may be N or C(R₆₁₄), X₆₁₅ may be N or C(R₆₁₅), X₆₁₆ may be N or C(R₆₁₆), and at least one of X₆₁₄ to X₆₁₆ may be N,
L₆₁₁ to L₆₁₃ are each independently as described in connection with L₆₀₁,
xe611 to xe613 are each independently as described in connection with xe1,
R₆₁₁ to R₆₁₃ are each independently as described in connection with R₆₀₁, and
R₆₁₄ to R₆₁₆ may each independently be hydrogen, deuterium, —F, —Cl, —Br, —I, —SF₅, a hydroxyl group, a cyano group, a nitro group, an amino group, an amidino group, a hydrazino group, a hydrazono group, a C₁-C₂₀ alkyl group, a C₁-C₂₀ alkoxy group, a C₁-C₂₀ alkylthio group, a phenyl group, a biphenyl group, a terphenyl group, or a naphthyl group.

In one or more embodiments, xe1 and xe611 to xe613 in Formulae 601 and 601-1 may each independently be 0, 1, or 2.

In one or more embodiments, R₆₀₁ and R₆₁₁ to R₆₁₃ in Formulae 601 and 601-1 may each independently be a phenyl group, a biphenyl group, a terphenyl group, a naphthyl group, a fluorenyl group, a spiro-bifluorenyl group, a benzofluorenyl group, a dibenzofluorenyl group, a phenanthrenyl group, an anthracenyl group, a fluoranthenyl group, a triphenylenyl group, a pyrenyl group, a chrysenyl group, a perylenyl group, a pentaphenyl group, a hexacenyl group, a pentacenyl group, a thiophenyl group, a furanyl group, a carbazolyl group, an indolyl group, an isoindolyl group, a benzofuranyl group, a benzothiophenyl group, a dibenzofuranyl group, a dibenzothiophenyl group, a benzocarbazolyl group, a dibenzocarbazolyl group, a dibenzosilolyl group, a pyridinyl group, an imidazolyl group, a pyrazolyl group, a thiazolyl group, an isothiazolyl group, an oxazolyl group, an isoxazolyl group, a thiadiazolyl group, an oxadiazolyl group, a pyrazinyl group, a pyrimidinyl group, a pyridazinyl group, a triazinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a phenanthridinyl group, an acridinyl group, a phenanthrolinyl group, a phenazinyl group, a benzimidazolyl group, an isobenzothiazolyl group, a benzoxazolyl group, an isobenzoxazolyl group, a triazolyl group, a tetrazolyl group, an imidazopyridinyl group, an imidazopyrimidinyl group, or an azacarbazolyl group, each unsubstituted or substituted with at least one of deuterium, —F, —Cl, —Br, —I, —SF₅, a hydroxyl group, a cyano group, a nitro group, an amino group, an amidino group, a hydrazino group, a hydrazono group, a C₁-C₂₀ alkyl group, a C₁-C₂₀ alkoxy group, a C₁-C₂₀ alkylthio group, a phenyl group, a biphenyl group, a terphenyl group, a naphthyl group, a fluorenyl group, a spiro-bifluorenyl group, a benzofluorenyl group, a dibenzofluorenyl group, a phenanthrenyl group, an anthracenyl group, a fluoranthenyl group, a triphenylenyl group, a pyrenyl group, a chrysenyl group, a perylenyl group, a pentaphenyl group, a hexacenyl group, a pentacenyl group, a thiophenyl group, a furanyl group, a carbazolyl group, an indolyl group, an isoindolyl group, a benzofuranyl group, a benzothiophenyl group, a dibenzofuranyl group, a dibenzothiophenyl group, a benzocarbazolyl group, a dibenzocarbazolyl group, a dibenzosilolyl group, a pyridinyl group, an imidazolyl group, a pyrazolyl group, a thiazolyl group, an isothiazolyl group, an oxazolyl group, an isoxazolyl group, a thiadiazolyl group, an oxadiazolyl group, a pyrazinyl group, a pyrimidinyl group, a pyridazinyl group, a triazinyl group, a quinolinyl group, an isoquinolinyl group, a benzoquinolinyl group, a phthalazinyl group, a naphthyridinyl group, a quinoxalinyl group, a quinazolinyl group, a cinnolinyl group, a phenanthridinyl group, an acridinyl group, a phenanthrolinyl group, a phenazinyl group, a benzimidazolyl group, an isobenzothiazolyl group, a benzoxazolyl group, an isobenzoxazolyl group, a triazolyl group, a tetrazolyl group, an imidazopyridinyl group, an imidazopyrimidinyl group, an azacarbazolyl group, or a combination thereof; or
—S(═O)₂(Q₆₀₁), —P(═O)(Q₆₀₁)(Q₆₀₂), —P(Q₆₀₁)(Q₆₀₂), and
Q₆₀₁ and Q₆₀₂ may each be as described herein.

The electron transport region 17 may include one of Compounds ET1 to ET36 or a combination thereof, but embodiments are not limited thereto:

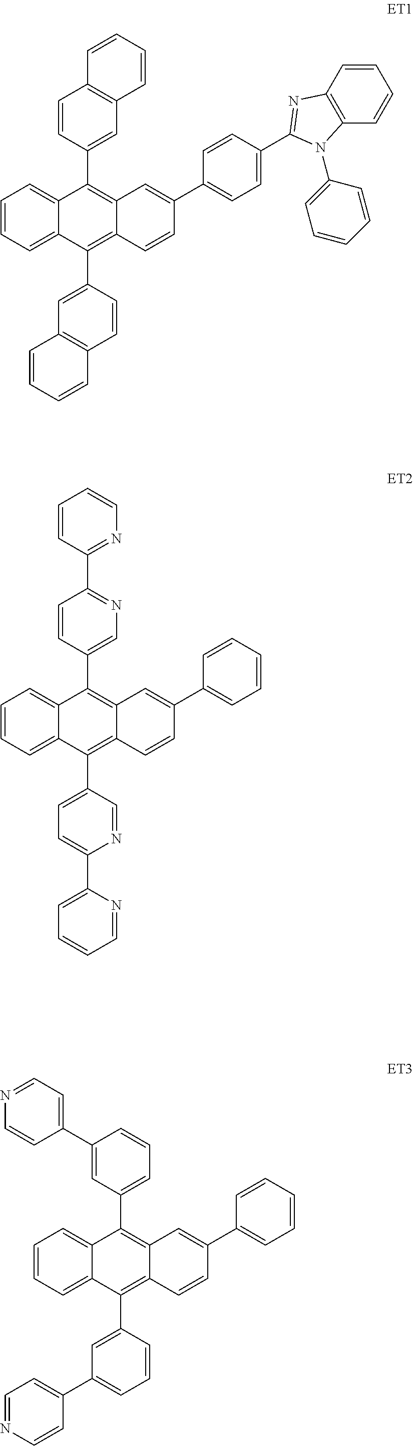 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

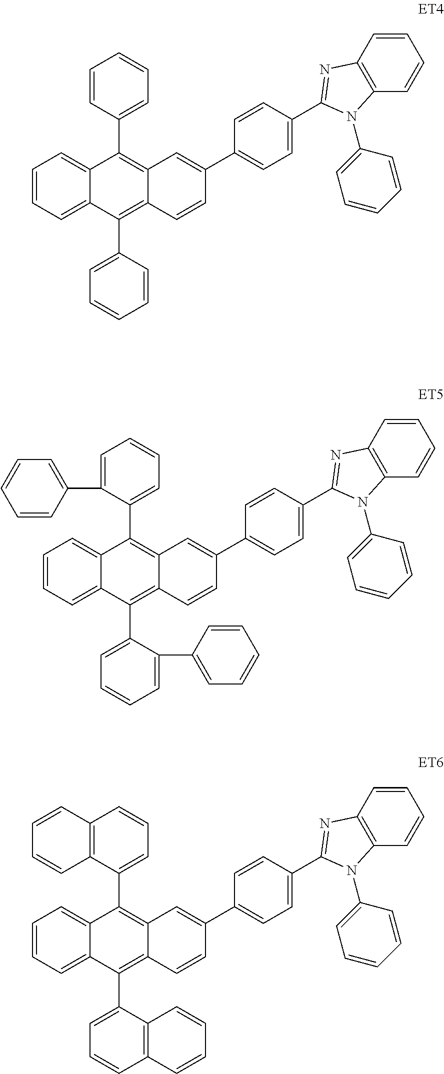 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1]

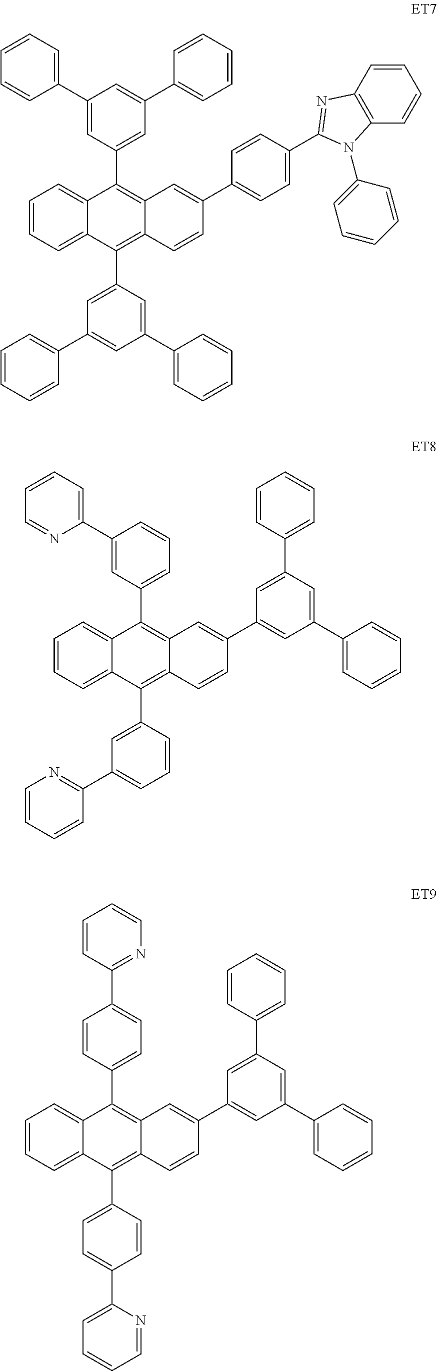 [SMILES: c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1 ; c1ccc(-c2cc(-c3ccccc3)cc(-c3ccc4c(-c5cccc(-c6ccccn6)c5)c5ccccc5c(-c5cccc(-c6ccccn6)c5)c4c3)c2)cc1 ; c1ccc(-c2cc(-c3ccccc3)cc(-c3ccc4c(-c5ccc(-c6ccccn6)cc5)c5ccccc5c(-c5ccc(-c6ccccn6)cc5)c4c3)c2)cc1]

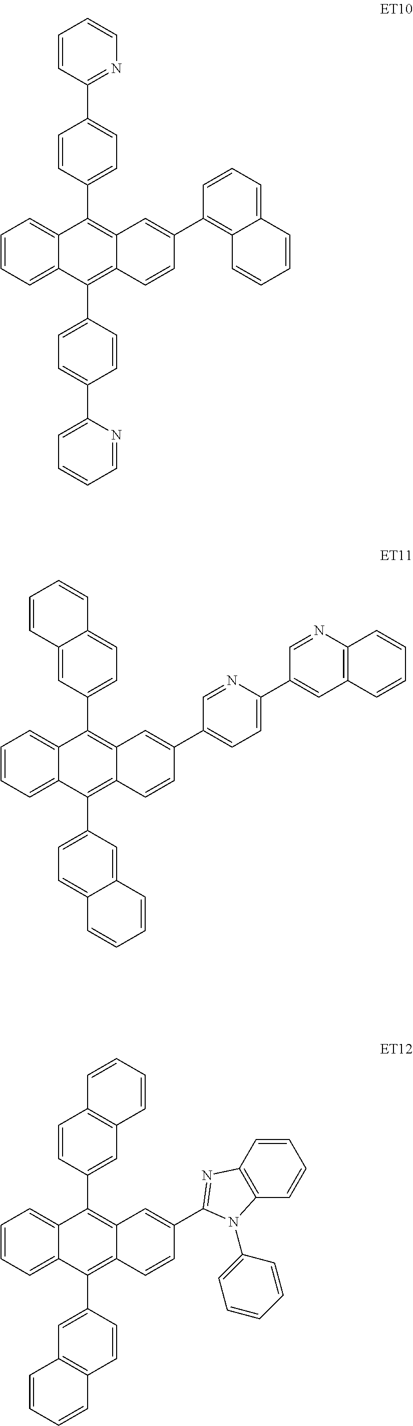 [SMILES: c1ccc(-c2ccc(-c3c4ccccc4c(-c4ccc(-c5ccccn5)cc4)c4cc(-c5cccc6ccccc56)ccc34)cc2)nc1 ; c1ccc2cc(-c3c4ccccc4c(-c4ccc5ccccc5c4)c4cc(-c5ccc(-c6cnc7ccccc7c6)nc5)ccc34)ccc2c1 ; c1ccc(-n2c(-c3ccc4c(-c5ccc6ccccc6c5)c5ccccc5c(-c5ccc6ccccc6c5)c4c3)nc3ccccc32)cc1]

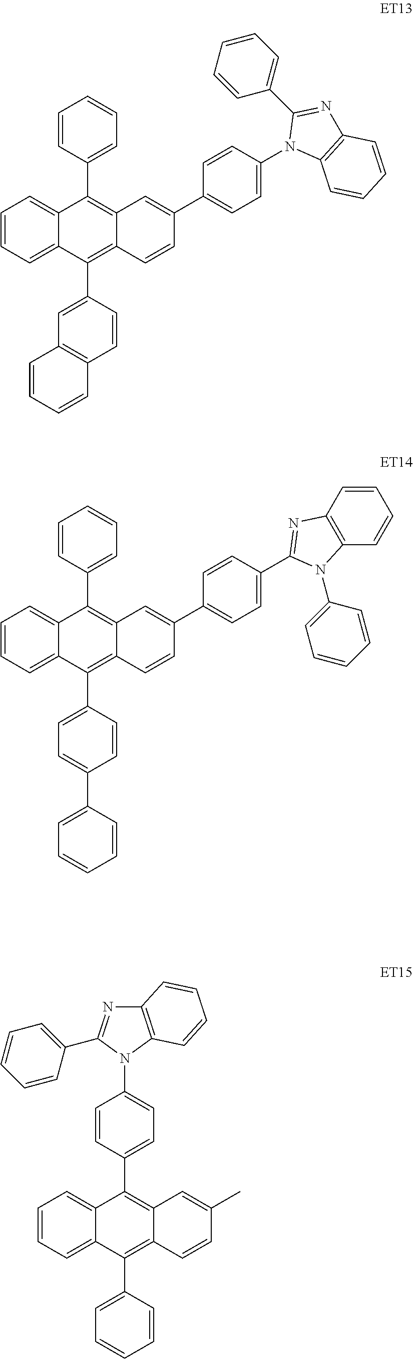 [SMILES: c1ccc(-c2c3ccccc3c(-c3ccc4ccccc4c3)c3ccc(-c4ccc(-n5c(-c6ccccc6)nc6ccccc65)cc4)cc23)cc1 ; c1ccc(-c2ccc(-c3c4ccccc4c(-c4ccccc4)c4cc(-c5ccc(-c6nc7ccccc7n6-c6ccccc6)cc5)ccc34)cc2)cc1 ; Cc1ccc2c(-c3ccccc3)c3ccccc3c(-c3ccc(-n4c(-c5ccccc5)nc5ccccc54)cc3)c2c1]

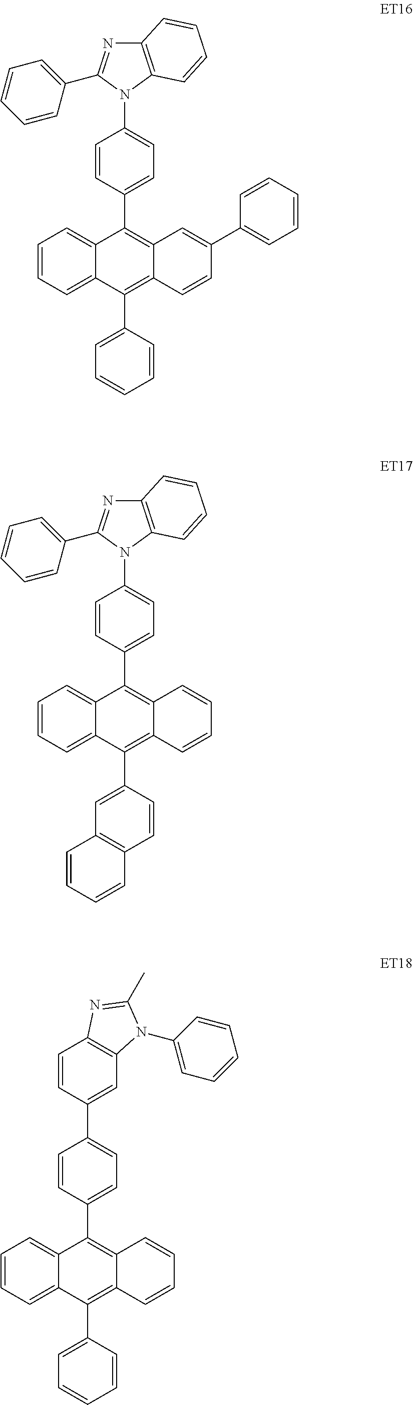 [SMILES: c1ccc(-c2ccc3c(-c4ccccc4)c4ccccc4c(-c4ccc(-n5c(-c6ccccc6)nc6ccccc65)cc4)c3c2)cc1 ; c1ccc(-c2nc3ccccc3n2-c2ccc(-c3c4ccccc4c(-c4ccc5ccccc5c4)c4ccccc34)cc2)cc1 ; Cc1nc2ccc(-c3ccc(-c4c5ccccc5c(-c5ccccc5)c5ccccc45)cc3)cc2n1-c1ccccc1]

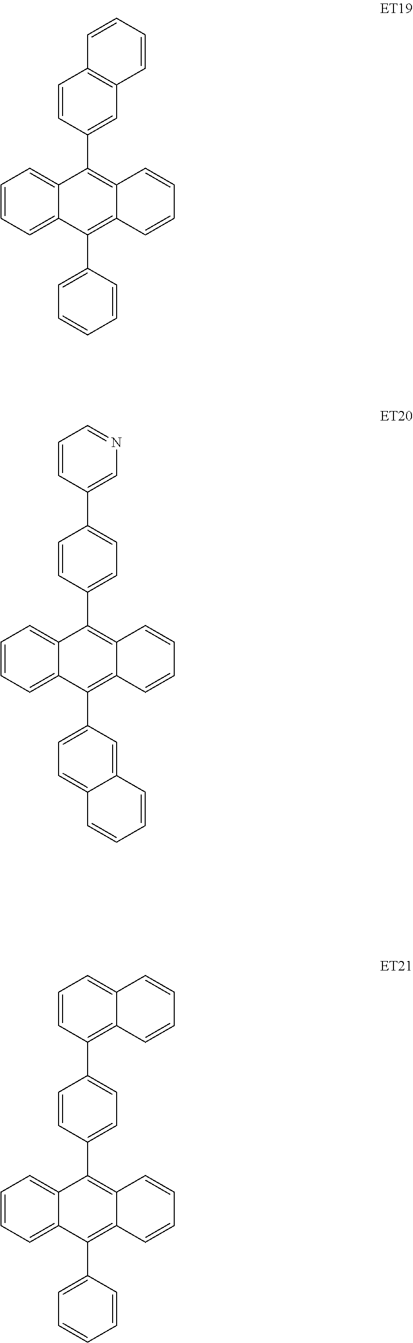

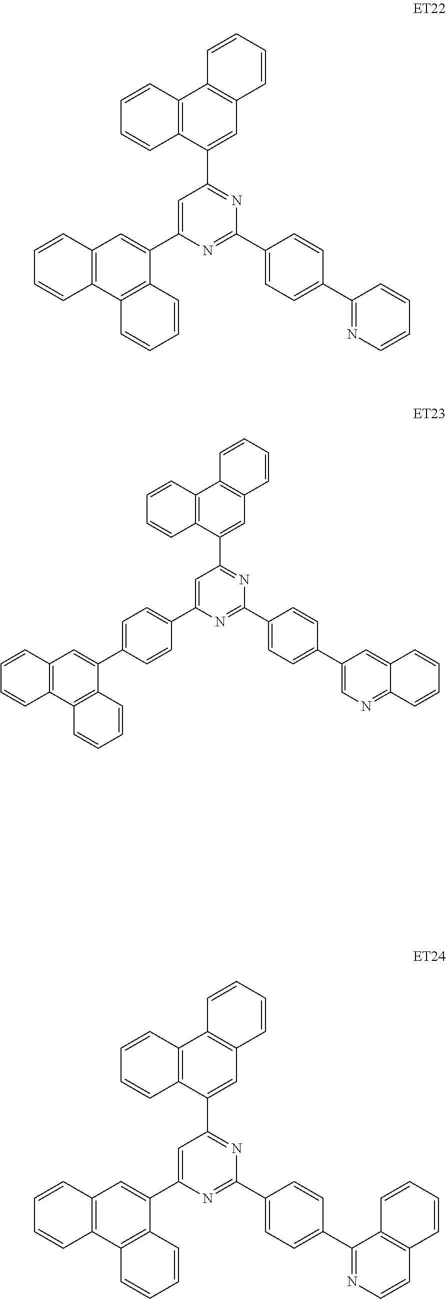 [SMILES: c1ccc(-c2ccc(-c3nc(-c4cc5ccccc5c5ccccc45)cc(-c4cc5ccccc5c5ccccc45)n3)cc2)nc1 ; c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1]

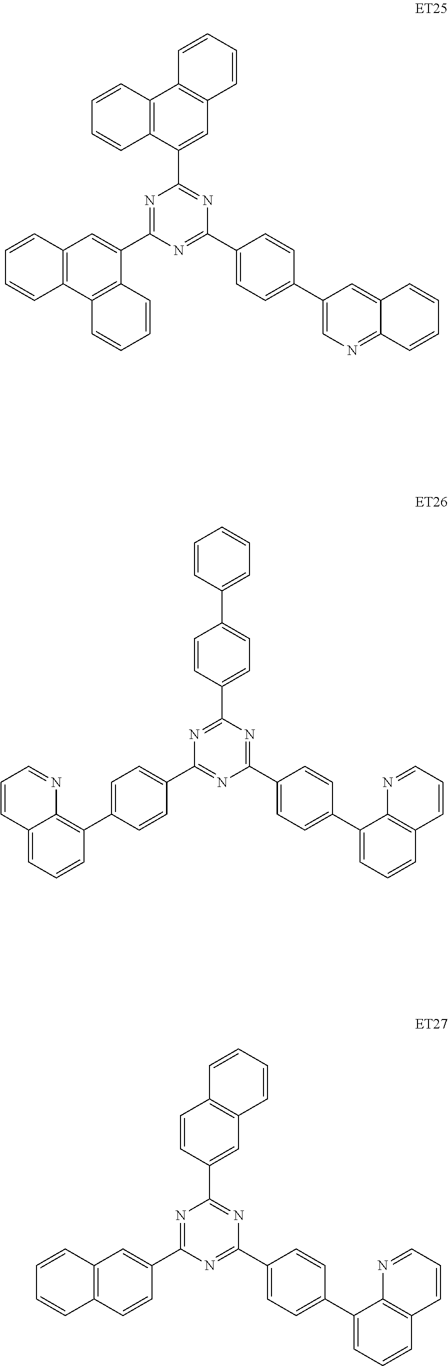 [SMILES: c1ccc2ncc(-c3ccc(-c4nc(-c5cc6ccccc6c6ccccc56)nc(-c5cc6ccccc6c6ccccc56)n4)cc3)cc2c1 ; c1ccc(-c2ccc(-c3nc(-c4ccc(-c5cccc6cccnc56)cc4)nc(-c4ccc(-c5cccc6cccnc56)cc4)n3)cc2)cc1 ; c1ccc2cc(-c3nc(-c4ccc(-c5cccc6cccnc56)cc4)nc(-c4ccc5ccccc5c4)n3)ccc2c1]

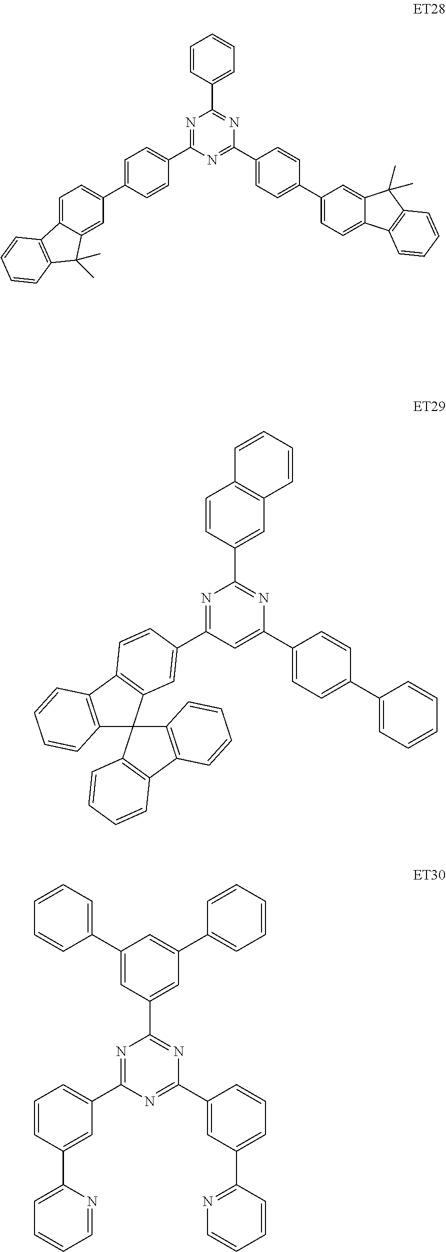 [SMILES: CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21 ; c1ccc(-c2ccc(-c3cc(-c4ccc5c(c4)C4(c6ccccc6-c6ccccc64)c4ccccc4-5)nc(-c4ccc5ccccc5c4)n3)cc2)cc1 ; c1ccc(-c2cc(-c3ccccc3)cc(-c3nc(-c4cccc(-c5ccccn5)c4)nc(-c4cccc(-c5ccccn5)c4)n3)c2)cc1]

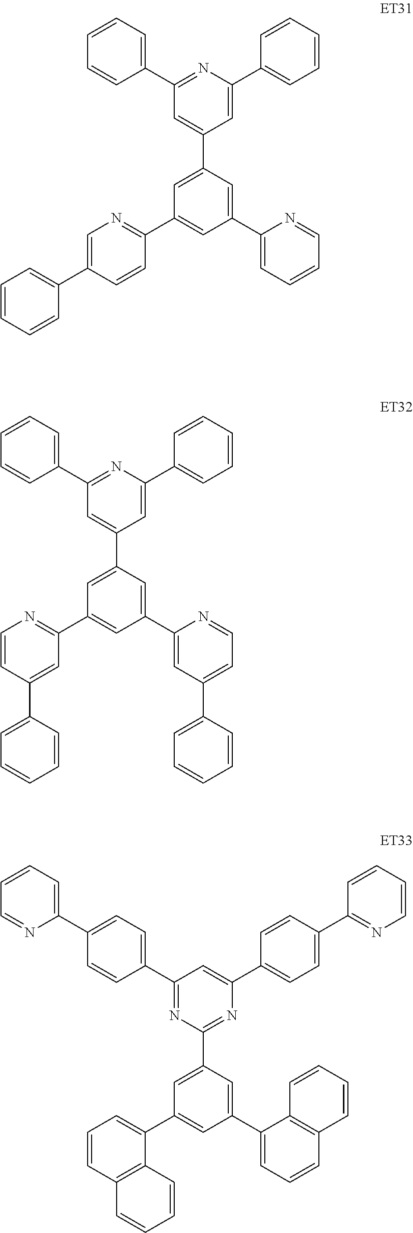 [SMILES: c1ccc(-c2ccc(-c3cc(-c4cc(-c5ccccc5)nc(-c5ccccc5)c4)cc(-c4ccccn4)c3)nc2)cc1 ; c1ccc(-c2ccnc(-c3cc(-c4cc(-c5ccccc5)nc(-c5ccccc5)c4)cc(-c4cc(-c5ccccc5)ccn4)c3)c2)cc1 ; c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1]

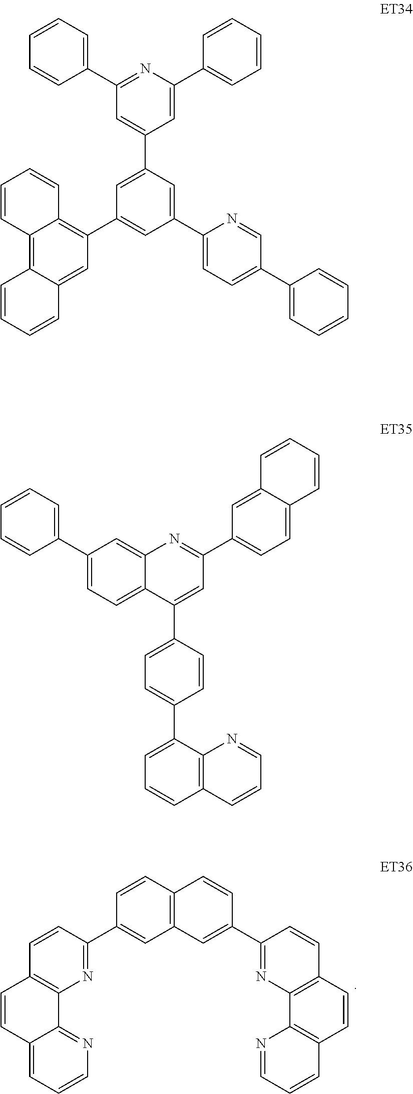 [SMILES: c1ccc(-c2ccc(-c3cc(-c4cc(-c5ccccc5)nc(-c5ccccc5)c4)cc(-c4cc5ccccc5c5ccccc45)c3)nc2)cc1 ; c1ccc(-c2ccc3c(-c4ccc(-c5cccc6cccnc56)cc4)cc(-c4ccc5ccccc5c4)nc3c2)cc1 ; c1cnc2c(c1)ccc1ccc(-c3ccc4ccc(-c5ccc6ccc7cccnc7c6n5)cc4c3)nc12]

In one or more embodiments, the electron transport region 17 may include 2,9-dimethyl-4,7-diphenyl-1,10-phenanthroline (BCP), 4,7-diphenyl-1,10-phenanthroline (Bphen), Alq₃, BAlq, 3-(biphenyl-4-yl)-5-(4-tert-butylphenyl)-4-phenyl-4H-1,2,4-triazole (TAZ), 4-(naphthalen-1-yl)-3,5-diphenyl-4H-1,2,4-triazole (NTAZ), 2,8-bis(diphenyl phosphineoxide)dibenzofuran (DBFPO), or a combination thereof. For example, when the electron transport region 17 includes a hole blocking layer, the hole blocking layer may include BCP or Bphen, but embodiments are not limited thereto:

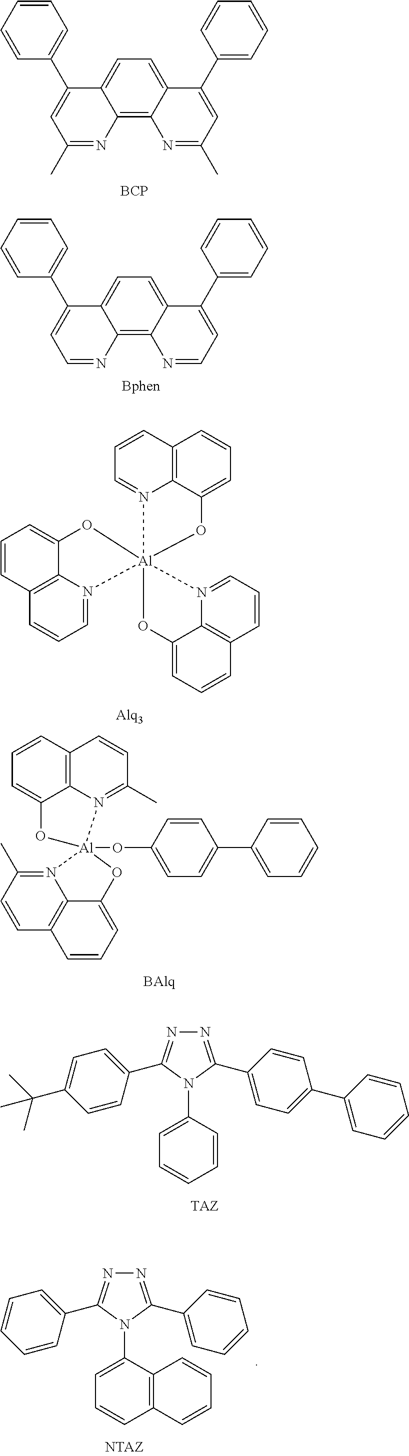

Thicknesses of the buffer layer, the hole blocking layer, and the electron control layer may each independently be about 20 Å to about 1,000 Å, for example, about 30 Å to about 300 Å. Without wishing to be bound to theory, when the thicknesses of the buffer layer, the hole blocking layer, and the electron control layer are within these ranges, excellent hole blocking characteristics or excellent electron control characteristics may be obtained without a substantial increase in driving voltage.

A thickness of the electron transport layer may be about 100 Å to about 1,000 Å, for example, about 150 Å to about 500 Å. Without wishing to be bound to theory, when the thickness of the electron transport layer is within these ranges, satisfactory electron transporting characteristics may be obtained without a substantial increase in driving voltage.

The electron transport region 17 (for example, the electron transport layer in the electron transport region 17) may further include, in addition to the aforementioned materials, a metal-containing material.

The metal-containing material may include an alkali metal complex, an alkaline earth metal complex, or a combination thereof. A metal ion of the alkali metal complex may include a Li ion, a Na ion, a K ion, a Rb ion, a Cs ion, or a combination thereof, and a metal ion of the alkaline earth metal complex may include a Be ion, a Mg ion, a Ca ion, a Sr ion, a Ba ion, or a combination thereof. A ligand coordinated with the metal ion of the alkali metal complex or the alkaline earth-metal complex may include a hydroxyquinoline, a hydroxyisoquinoline, a hydroxybenzoquinoline, a hydroxyacridine, a hydroxyphenanthridine, a hydroxyphenyloxazole, a hydroxyphenylthiazole, a hydroxydiphenyloxadiazole, a hydroxydiphenylthiadiazole, a hydroxyphenylpyridine, a hydroxyphenylbenzimidazole, a hydroxyphenylbenzothiazole, a bipyridine, a phenanthroline, a cyclopentadiene, or a combination thereof.

In one or more embodiments, the metal-containing material may include a Li complex. The Li complex may include, for example, Compound ET-D1 (LiQ) or ET-D2, but embodiments are not limited thereto:

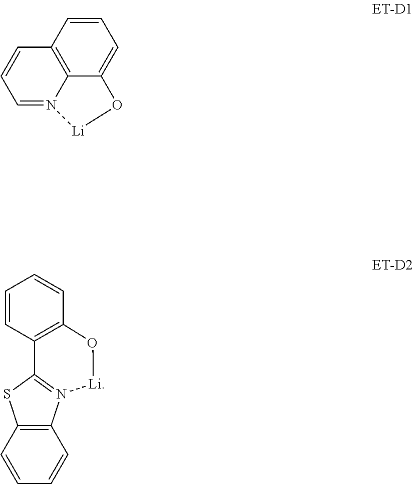

The electron transport region 17 may include an electron injection layer that facilitates the injection of electrons from the second electrode 19. The electron injection layer may directly contact the second electrode 19.

The electron injection layer may have i) a single-layer structure consisting of a single layer including a single material, ii) a single-layer structure consisting of a single layer including multiple materials that are different from each other, or iii) a multi-layer structure consisting of multiple layers including multiple materials that are different from each other.

The electron injection layer may include an alkali metal, an alkaline earth metal, a rare earth metal, an alkali metal compound, an alkaline earth-metal compound, a rare earth metal compound, an alkali metal complex, an alkaline earth-metal complex, a rare earth metal complex, or a combination thereof.

The alkali metal may include Li, Na, K, Rb, Cs, or a combination thereof. In one or more embodiments, the alkali metal may be Li, Na, or Cs. In one or more embodiments, the alkali metal may be Li or Cs.

The alkaline earth metal may include Mg, Ca, Sr, Ba, or a combination thereof.

The rare earth metal may include Sc, Y, Ce, Tb, Yb, Gd, or a combination thereof.

The alkali metal compound, the alkaline earth metal compound, and the rare earth metal compound may include oxides and halides (for example, fluorides, chlorides, bromides, or iodides) of the alkali metal, the alkaline earth metal, and the rare earth metal, or a combination thereof.

The alkali metal compound may include one of alkali metal oxides such as Li₂O, Cs₂O, K₂O, or the like, or a combination thereof; one of alkali metal halides such as LiF, NaF, CsF, KF, LiI, NaI, CsI, KI, or the like, or a combination thereof; or a combination thereof. In one or more embodiments, the alkali metal compound may include LiF, Li₂O, NaF, LiI, NaI, CsI, KI, or a combination thereof.

The alkaline earth-metal compound may include one of alkaline earth-metal compounds, such as BaO, SrO, CaO, BaₓSr₁₋ₓO (wherein 0<x<1), or BaₓCa₁₋ₓO (wherein 0<x<1), or the like, or a combination thereof. In one or more embodiments, the alkaline earth metal compound may include BaO, SrO, CaO, or the like, or a combination thereof.

The rare earth metal compound may include YbF₃, ScF₃, Sc₂O₃, Y₂O₃, Ce₂O₃, GdF₃, TbF₃, or the like, or a combination thereof. In one or more embodiments, the rare earth metal compound may include YbF₃, ScF₃, TbF₃, YbI₃, ScI₃, TbI₃, or the like, or a combination thereof.

The alkali metal complex, the alkaline earth metal complex, and the rare earth metal complex may include an ion of an alkali metal, an alkaline earth metal, and/or a rare earth metal as described above, and a ligand coordinated with a metal ion of the alkali metal complex, the alkaline earth metal complex, or the rare earth metal complex may include hydroxy quinoline, hydroxy isoquinoline, hydroxy benzoquinoline, hydroxy acridine, hydroxy phenanthridine, hydroxy phenyloxazole, hydroxy phenylthiazole, hydroxy diphenyloxadiazole, hydroxy diphenylthiadiazole, hydroxy phenylpyridine, hydroxy phenylbenzimidazole, hydroxy phenylbenzothiazole, bipyridine, phenanthroline, cyclopentadiene, or the like, or a combination thereof.

The electron injection layer may consist of an alkali metal, an alkaline earth metal, a rare earth metal, an alkali metal compound, an alkaline earth-metal compound, a rare earth metal compound, an alkali metal complex, an alkaline earth-metal complex, a rare earth metal complex, or a combinations thereof, as described above. In one or more embodiments, the electron injection layer may further include an organic material. When the electron injection layer further includes an organic material, an alkali metal, an alkaline earth metal, a rare earth metal, an alkali metal compound, an alkaline earth-metal compound, a rare earth metal compound, an alkali metal complex, an alkaline earth-metal complex, a rare earth metal complex, or a combination thereof may be homogeneously or non-homogeneously dispersed in a matrix including the organic material.

A thickness of the electron injection layer may be about 1 Å to about 100 Å, and, for example, about 3 Å to about 90 Å. Without wishing to be bound to theory, when the thickness of the electron injection layer is within these ranges, satisfactory electron injection characteristics may be obtained without a substantial increase in driving voltage.

### Second Electrode 19]

The second electrode 19 may be arranged on the aforementioned organic layer 10A. The second electrode 19 may be a cathode which is an electron injection electrode, and in this regard, a material for forming the second electrode 19 may be selected from a metal, an alloy, an electrically conductive compound, and a combination thereof, which have a relatively low work function.

The second electrode 19 may include Li, Ag, Mg, Al, Al—Li, Ca, Mg—In, Mg—Ag, ITO, IZO, or a combination thereof. The second electrode 19 may be a transmissive electrode, a semi-transmissive electrode, or a reflective electrode.

The second electrode 19 may have a single-layer structure having a single layer or a multi-layer structure including two or more layers.

### Explanation of Terms

The term “C₁-C₆₀ alkyl group” as used herein refers to a linear or branched saturated aliphatic hydrocarbons monovalent group having 1 to 60 carbon atoms, and the term “C₁-C₆₀ alkylene group, as used here refers to a divalent group having the same structure as the C₁-C₆₀ alkyl group.

Non-limiting examples of the C₁-C₆₀ alkyl group, the C₁-C₂₀ alkyl group, and/or the C₁-C₁₀ alkyl group include a methyl group, an ethyl group, an n-propyl group, an isopropyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, an n-hexyl group, an isohexyl group, a sec-hexyl group, a tert-hexyl group, an n-heptyl group, an isoheptyl group, a sec-heptyl group, a tert-heptyl group, an n-octyl group, an isooctyl group, a sec-octyl group, a tert-octyl group, an n-nonyl group, an isononyl group, a sec-nonyl group, a tert-nonyl group, an n-decyl group, an isodecyl group, a sec-decyl group, or a tert-decyl group, each unsubstituted or substituted with at least one of a methyl group, an ethyl group, an n-propyl group, an isopropyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, an n-hexyl group, an isohexyl group, a sec-hexyl group, a tert-hexyl group, an n-heptyl group, an isoheptyl group, a sec-heptyl group, a tert-heptyl group, an n-octyl group, an isooctyl group, a sec-octyl group, a tert-octyl group, an n-nonyl group, an isononyl group, a sec-nonyl group, a tert-nonyl group, an n-decyl group, an isodecyl group, a sec-decyl group, a tert-decyl group, or the like, or a combination thereof.

The term “C₁-C₆₀ alkoxy group” as used herein refers to a monovalent group represented by —OA₁₀₁ (wherein A₁₀₁ is the C₁-C₆₀ alkyl group), and non-limiting examples thereof include a methoxy group, an ethoxy group, a propoxy group, a butoxy group, a pentoxy group, or the like.

The term “C₁-C₆₀ alkylthio group” as used herein refers to a monovalent group represented by —SA₁₀₁, (wherein A₁₀₁, is the C₁-C₆₀ alkyl group).

The term “C₂-C₆₀ alkenyl group” as used herein refers to a hydrocarbon group formed by substituting at least one carbon-carbon double bond in the middle or at the terminus of the C₂-C₆₀ alkyl group, and non-limiting examples thereof include an ethenyl group, a propenyl group, a butenyl group, or the like. The term “C₂-C₆₀ alkenylene group” as used herein refers to a divalent group having the same structure as the C₂-C₆₀ alkenyl group.

The term “C₂-C₆₀ alkynyl group” as used herein refers to a hydrocarbon group formed by substituting at least one carbon-carbon triple bond in the middle or at the terminus of the C₂-C₆₀ alkyl group, and non-limiting examples thereof include an ethynyl group, a propynyl group, or the like. The term “C₂-C₆₀ alkynylene group” as used herein refers to a divalent group having the same structure as the C₂-C₆₀ alkynyl group.

The term “C₃-C₁₀ cycloalkyl group” as used herein refers to a monovalent saturated hydrocarbon ring group having 3 to 10 carbon atoms, and the term “C₃-C₁₀ cycloalkylene group” as used herein refers to a divalent group having the same structure as the C₃-C₁₀ cycloalkyl group.

Non-limiting examples of the C₃-C₁₀ cycloalkyl group include a cyclopropyl group, a cyclobutyl group, a cyclopentyl group, cyclohexyl group, a cycloheptyl group, a cyclooctyl group, an adamantanyl group, a norbornanyl group (a bicyclo[2.2.1]heptyl group), a bicyclo[1.1.1]pentyl group, a bicyclo[2.1.1]hexyl group, a bicyclo[2.2.2]octyl group, or the like.

The term “C₁-C₁₀ heterocycloalkyl group” as used herein refers to a monovalent monocyclic group that includes at least one heteroatom selected from N, O, P, Si, S, Se, Ge, and B as a ring-forming atom and 1 to 10 carbon atoms as ring-forming atom(s), and the term “the C₁-C₁₀ heterocycloalkylene group” as used herein refers to a divalent group having the same structure as the C₁-C₁₀ heterocycloalkyl group.

Non-limiting examples of the C₁-C₁₀ heterocycloalkyl group include a silolanyl group, a silinanyl group, tetrahydrofuranyl group, a tetrahydro-2H-pyranyl group, a tetrahydrothiophenyl group, or the like.

The term “C₃-C₁₀ cycloalkenyl group” as used herein refers to a monovalent cyclic group that includes 3 to 10 carbon atoms and at least one carbon-carbon double bond in the ring thereof and has no aromaticity, and non-limiting examples thereof include a cyclopentenyl group, a cyclohexenyl group, a cycloheptenyl group, or the like. The term “C₃-C₁₀ cycloalkenylene group” as used herein refers to a divalent group having the same structure as the C₃-C₁₀ cycloalkenyl group.

The term “C₁-C₁₀ heterocycloalkenyl group” as used herein refers to a monovalent monocyclic group that has at least one heteroatom selected from N, O, P, Si, S, Se, Ge, and B as a ring-forming atom, 1 to 10 carbon atoms as ring-forming atom(s), and at least one double bond in the ring thereof. Non-limiting examples of the C₁-C₁₀ heterocycloalkenyl group include a 2,3-dihydrofuranyl group, a 2,3-dihydrothiophenyl group, or the like. The term “C₁-C₁₀ heterocycloalkenylene group” as used herein refers to a divalent group having the same structure as the C₁-C₁₀ heterocycloalkenyl group.

The term “C₆-C₆₀ aryl group” as used herein refers to a monovalent group having a carbocyclic aromatic ring system having 6 to 60 carbon atoms, and the term “C₆-C₆₀ arylene group” as used herein refers to a divalent group having a carbocyclic aromatic ring system having 6 to 60 carbon atoms. Non-limiting examples of the C₆-C₆₀ aryl group include a phenyl group, a naphthyl group, an anthracenyl group, a phenanthrenyl group, a pyrenyl group, a chrysenyl group, or the like. When the C₆-C₆₀ aryl group and the C₆-C₆₀ arylene group each include two or more rings, the rings may be fused to each other.

The term “C₇-C₆₀ alkyl aryl group” as used herein refers to a C₆-C₆₀ aryl group substituted with at least one C₁-C₆₀ alkyl group. The term “C₇-C₆₀ aryl alkyl group” as used herein refers to a C₁-C₆₀ alkyl group substituted with at least one C₆-C₆₀ aryl group.

The term “C₁-C₆₀ heteroaryl group” as used herein refers to a monovalent group that includes at least one heteroatom selected from N, O, P, Si, S, Se, Ge, and B as a ring-forming atom and a heterocyclic aromatic ring system having 1 to 60 carbon atoms as ring-forming atom(s), and the term “C₁-C₆₀ heteroarylene group” as used herein refers to a divalent group that includes at least one heteroatom selected from N, O, P, Si, S, Se, Ge, and B as a ring-forming atom and a heterocyclic aromatic ring system having 1 to 60 carbon atoms as ring-forming atom(s).

Non-limiting examples of the C₁-C₆₀ heteroaryl group include a pyridinyl group, a pyrimidinyl group, a pyrazinyl group, a pyridazinyl group, a triazinyl group, a quinolinyl group, an isoquinolinyl group, or the like. When the C₁-C₆₀ heteroaryl group and the C₁-C₆₀ heteroarylene group each include two or more rings, the rings may be fused to each other.

The term “C₂-C₆₀ alkyl heteroaryl group” as used herein refers to a C₁-C₆₀ heteroaryl group substituted with at least one C₁-C₆₀ alkyl group. The term “C₂-C₆₀ heteroaryl alkyl group” as used herein refers to a C₁-C₆₀ alkyl group substituted with at least one C₁-C₆₀ heteroaryl group.

The term “C₆-C₆₀ aryloxy group” as used herein indicates —OA₁₀₂ (wherein A₁₀₂ is the C₆-C₆₀ aryl group), and the term “C₆-C₆₀ arylthio group” as used herein indicates —SA₁₀₃ (wherein A₁₀₃ is the C₆-C₆₀ aryl group).

The term “C₁-C₆₀ heteroaryloxy group” as used herein indicates —OA_{102′} (wherein A_{102′} is the C₁-C₆₀ heteroaryl group), and the term “C₁-C₆₀ heteroarylthio group” as used herein indicates —SA_{103′} (wherein A_{103′} is the C₁-C₆₀ heteroaryl group).

The term “monovalent non-aromatic condensed polycyclic group” as used herein refers to a monovalent group in which two or more rings are condensed with each other, only carbon is used as a ring-forming atom (for example, the number of carbon atoms may be 8 to 60), and the whole molecule is a non-aromatic group. Non-limiting examples of the monovalent non-aromatic condensed polycyclic group include a fluorenyl group or the like. The term “divalent non-aromatic condensed polycyclic group” as used herein refers to a divalent group having the same structure as the monovalent non-aromatic condensed polycyclic group described above.

The term “monovalent non-aromatic condensed heteropolycyclic group” as used herein refers to a monovalent group having two or more rings condensed with each other, a heteroatom selected from N, O, P, Si, S, Se, Ge, and B, other than carbon atoms (for example, having 1 to 60 carbon atoms), as a ring-forming atom, and no aromaticity in the entire molecular structure thereof. Non-limiting examples of the monovalent non-aromatic condensed heteropolycyclic group include a carbazolyl group or the like. The term “divalent non-aromatic condensed heteropolycyclic group” as used herein refers to a divalent group having the same structure as the monovalent non-aromatic condensed heteropolycyclic group described above.

The term “π electron-depleted nitrogen-containing C₁-C₆₀ cyclic group” as used herein refers to a ring group having 1 to 60 carbon atoms and including at least one *—N═*′ (wherein * and *′ each indicate a binding site to a neighboring atom) as a ring-forming moiety. For example, the π electron-depleted nitrogen-containing C₁-C₆₀ cyclic group may be a) a first ring, b) a condensed ring in which at least two first rings are condensed, or c) a condensed ring in which at least one first ring and at least one second ring are condensed.

The term “π electron-rich C₃-C₆₀ cyclic group” as used herein refers to a ring group having 3 to 60 carbon atoms and not including at least one *—N═*′ (wherein * and *′ each indicate a binding site to a neighboring atom) as a ring-forming moiety. For example, the π electron-rich C₃-C₆₀ cyclic group may be a) a second ring or b) a condensed ring in which at least two second rings are condensed.

The term “C₅-C₆₀ cyclic group” as used herein refers to a monocyclic or polycyclic group having 5 to 60 carbon atoms, and may be, for example, a) a third ring or b) a condensed ring group in which at least two third rings are condensed to each other.

The “C₁-C₆₀ heterocyclic group” as used herein refers to a monocyclic or polycyclic group including at least one heteroatom and 1 to 60 carbon atoms, and may be, for example, a) a fourth ring, b) a condensed ring in which at least two fourth rings are condensed, or c) a condensed ring in which at least one third ring is condensed with at least one fourth ring.

The “first ring” as used herein may be an imidazole group, a pyrazole group, a thiazole group, an isothiazole group, an oxazole group, an isoxazole group, a pyridine group, a pyrazine group, a pyridazine group, a pyrimidine group, a triazole group, a tetrazole group, an oxadiazole group, a triazine group, or a thiadiazole group.

The “second ring” as used herein may be a benzene group, a cyclopentadiene group, a pyrrole group, a furan group, a thiophene group, or a silole group.

The “third ring” as used herein may be a cyclopentane group, a cyclopentadiene group, an indene group, an adamantane group, a norbornene group, a bicyclo[1.1.1]pentane group, a bicyclo[2.1.1]hexane group, a bicyclo[2.2.1]heptane group (a norbornane group), a bicyclo[2.2.2]octane group, a cyclohexane group, a cyclohexene group, or a benzene group.

The “fourth ring” as used herein may be a furan group, a thiophene group, a pyrrole group, a silole group, an oxazole group, an isoxazole group, an oxadiazole group, an isoxadiazole group, oxatriazole group, an isoxatriazole group, a thiazole group, an isothiazole group, a thiadiazole group, an isothiadiazole group, a thiatriazole group, an isotriazole group, a pyrazole group, an imidazole group, a triazole group, a tetrazole group, an azasilole group, a diazasilole group, a triazasilole group, a pyridine group, a pyrimidine group, a pyrazine group, a pyridazine group, or a triazine group.

For example, the π electron-depleted nitrogen-containing C₁-C₆₀ cyclic group may be an imidazole group, a pyrazole group, a thiazole group, an isothiazole group, an oxazole group, an isoxazole group, a pyridine group, a pyrazine group, a pyridazine group, a pyrimidine group, an indazole group, a purine group, a quinoline group, an isoquinoline group, a benzoquinoline group, a benzoisoquinoline group, a phthalazine group, a naphthyridine group, a quinoxaline group, a benzoquinoxaline group, a quinazoline group, a cinnoline group, a phenanthridine group, an acridine group, a phenanthroline group, a phenazine group, a benzimidazole group, an isobenzothiazole group, a benzoxazole group, an isobenzoxazole group, a triazole group, a tetrazole group, an oxadiazole group, a triazine group, a thiadiazole group, an imidazopyridine group, an imidazopyrimidine group, an azacarbazole group, an azadibenzofuran group, an azadibenzothiophene group, an azadibenzosilole group, an acridine group, or a pyridinopyrazole group.

For example, the π electron-rich C₃-C₆₀ cyclic group may be a benzene group, a heptalene group, an indene group, a naphthalene group, an azulene group, an indacene group, an acenaphthylene group, a fluorene group, a spiro-bifluorene group, a benzofluorene group, a dibenzofluorene group, a phenalene group, a phenanthrene group, an anthracene group, a fluoranthene group, a triphenylene group, a pyrene group, a chrysene group, a naphthacene group, a picene group, a perylene group, a pentacene group, a hexacene group, a pentaphene group, a rubicene group, a coronene group, an ovalene group, a pyrrole group, a furan group, a thiophene group, an isoindole group, an indole group, an indene group, a benzofuran group, a benzothiophene group, a benzosilole group, a naphthopyrrole group, a naphthofuran group, a naphthothiophene group, a naphthosilole group, a benzocarbazole group, a dibenzocarbazole group, a dibenzofuran group, a dibenzothiophene group, a carbazole group, a dibenzosilole group, an indenocarbazole group, an indolocarbazole group, a benzofurocarbazole group, a benzothienocarbazole group, a benzosilolocarbazole group, a triindolobenzene group, a pyrrolophenanthrene group, a furanophenanthrene group, a thienophenanthrene group, a benzonaphthofuran group, a benzonaphthothiophene group, an (indolo)phenanthrene group, a (benzofurano)phenanthrene group, or a (benzothieno)phenanthrene group.

For example, the C₅-C₆₀ carbocyclic group may be a cyclopentane group, a cyclohexane group, a cyclohexene group, a benzene group, a naphthalene group, an anthracene group, a phenanthrene group, a triphenylene group, a pyrene group, a chrysene group, a 1,2,3,4-tetrahydronaphthalene group, cyclopentadiene group, an indene group, a fluorene group, a 5,6,7,8-tetrahydroisoquinoline group, a 5,6,7,8-tetrahydroquinoline group, an adamantane group, a norbornane group, or a norbornene group.

For example, the C₁-C₆₀ heterocyclic group may be a thiophene group, a furan group, a pyrrole group, a cyclopentadiene group, a silole group, a borole group, a phosphole group, a selenophene group, a germole group, a benzothiophene group, a benzofuran group, an indole group, an indene group, a benzosilole group, a benzoborole group, a benzophosphole group, a benzoselenophene group, a benzogermole group, a dibenzothiophene group, a dibenzofuran group, a carbazole group, a dibenzosilole group, a dibenzoborole group, a dibenzophosphole group, a dibenzoselenophene group, a dibenzogermole group, a dibenzothiophene 5-oxide group, a 9H-fluorene-9-one group, a dibenzothiophene 5,5-dioxide group, an azabenzothiophene group, an azabenzofuran group, an azaindole group, an azaindene group, an azabenzosilole group, an azabenzoborole group, an azabenzophosphole group, an azabenzoselenophene group, an azabenzogermole group, an azadibenzothiophene group, an azadibenzofuran group, an azacarbazole group, an azafluorene group, an azadibenzosilole group, an azadibenzoborole group, an azadibenzophosphole group, an azadibenzoselenophene group, an azadibenzogermole group, an azadibenzothiophene 5-oxide group, an aza-9H-fluorene-9-one group, an azadibenzothiophene 5,5-dioxide group, a pyridine group, a pyrimidine group, a pyrazine group, a pyridazine group, a triazine group, a quinoline group, an isoquinoline group, a quinoxaline group, a quinazoline group, a phenanthroline group, a pyrazole group, an imidazole group, a triazole group, an oxazole group, an isoxazole group, a thiazole group, an isothiazole group, an oxadiazole group, a thiadiazole group, a benzopyrazole group, a benzimidazole group, a benzoxazole group, a benzothiazole group, a benzoxadiazole group, or a benzothiadiazole group.

The terms “a π electron-deficient nitrogen-containing C₁-C₆₀ cyclic group, a π electron-rich C₃-C₆₀ cyclic group, a C₅-C₆₀ cyclic group, and a C₁-C₆₀ heterocyclic group” as used herein each refer to a part of a condensed ring or a monovalent, a divalent, a trivalent, a tetravalent, a pentavalent, or a hexavalent ring group, depending on the formula structure.

Substituents of the substituted π electron-deficient nitrogen-containing C₁-C₆₀ cyclic group, the substituted π electron-rich C₃-C₆₀ cyclic group, the substituted C₅-C₆₀ cyclic group, the substituted C₁-C₆₀ heterocyclic group, the substituted C₁-C₆₀ alkylene group, the substituted C₂-C₆₀ alkenylene group, the substituted C₂-C₆₀ alkynylene group, the substituted C₃-C₁₀ cycloalkylene group, the substituted C₁-C₁₀ heterocycloalkylene group, the substituted C₃-C₁₀ cycloalkenylene group, the substituted C₁-C₁₀ heterocycloalkenylene group, the substituted C₆-C₆₀ arylene group, the substituted C₁-C₆₀ heteroarylene group, the substituted divalent non-aromatic condensed polycyclic group, the substituted divalent non-aromatic condensed heteropolycyclic group, the substituted C₁-C₆₀ alkyl group, the substituted C₂-C₆₀ alkenyl group, the substituted C₂-C₆₀ alkynyl group, the substituted C₁-C₆₀ alkoxy group, the substituted C₁-C₆₀ alkylthio group, the substituted C₃-C₁₀ cycloalkyl group, the substituted C₁-C₁₀ heterocycloalkyl group, the substituted C₃-C₁₀ cycloalkenyl group, the substituted C₁-C₁₀ heterocycloalkenyl group, the substituted C₆-C₆₀ aryl group, the substituted C₇-C₆₀ alkyl aryl group, the substituted C₇-C₆₀ aryl alkyl group, the substituted C₆-C₆₀ aryloxy group, the substituted C₆-C₆₀ arylthio group, the substituted C₁-C₆₀ heteroaryl group, the substituted C₂-C₆₀ alkyl heteroaryl group, the substituted C₂-C₆₀ heteroaryl alkyl group, the substituted C₁-C₆₀ heteroaryloxy group, the substituted C₁-C₆₀ heteroarylthio group, the substituted monovalent non-aromatic condensed polycyclic group, and the substituted monovalent non-aromatic condensed heteropolycyclic group may each independently be:
deuterium, —F, —Cl, —Br, —I, —SF₅, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, or a C₁-C₆₀ alkylthio group;
a C₁-C₆₀ alkyl group, a C₂-C₆₀ alkenyl group, a C₂-C₆₀ alkynyl group, a C₁-C₆₀ alkoxy group, or a C₁-C₆₀ alkylthio group, each substituted with at least one of deuterium, —F, —Cl, —Br, —I, —SF₅, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₇-C₆₀ alkyl aryl group, a C₆-C₆₀ aryloxy group, a C₆-C₆₀ arylthio group, a C₁-C₆₀ heteroaryl group, a C₂-C₆₀ alkyl heteroaryl group, a C₁-C₆₀ heteroaryloxy group, a C₁-C₆₀ heteroarylthio group, a monovalent non-aromatic condensed polycyclic group, a monovalent non-aromatic condensed heteropolycyclic group, —N(Q₁₁)(Q₁₂), —Si(Q₁₃)(Q₁₄)(Q₁₅), —Ge(Q₁₃)(Q₁₄)(Q₁₅), —B(Q₁₆)(Q₁₇), —P(═O)(Q₁₈)(Q₁₉), —P(Q₁₈)(Q₁₉), or a combination thereof;
a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₇-C₆₀ alkyl aryl group, a C₆-C₆₀ aryloxy group, a C₆-C₆₀ arylthio group, a C₁-C₆₀ heteroaryl group, a C₂-C₆₀ alkyl heteroaryl group, a C₁-C₆₀ heteroaryloxy group, a C₁-C₆₀ heteroarylthio group, a monovalent non-aromatic condensed polycyclic group, or a monovalent non-aromatic condensed heteropolycyclic group, each unsubstituted or substituted with at least one of deuterium, —F, —Cl, —Br, —I, —SF₅, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₁-C₆₀ alkylthio group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₇-C₆₀ alkyl aryl group, a C₇-C₆₀ aryl alkyl group, a C₆-C₆₀ aryloxy group, a C₆-C₆₀ arylthio group, a C₁-C₆₀ heteroaryl group, a C₂-C₆₀ alkyl heteroaryl group, a C₂-C₆₀ heteroaryl alkyl group, a C₁-C₆₀ heteroaryloxy group, a C₁-C₆₀ heteroarylthio group, a monovalent non-aromatic condensed polycyclic group, a monovalent non-aromatic condensed heteropolycyclic group, —N(Q₂₁)(Q₂₂), —Si(Q₂₃)(Q₂₄)(Q₂₅), —Ge(Q₂₃)(Q₂₄)(Q₂₅), —B(Q₂₆)(Q₂₇), —P(═O)(Q₂₈)(Q₂₉), —P(Q₂₈)(Q₂₉), or a combination thereof;
—N(Q₃₁)(Q₃₂), —Si(Q₃₃)(Q₃₄)(Q₃₅), —Ge(Q₃₃)(Q₃₄)(Q₃₅), —B(Q₃₆)(Q₃₇), —P(═O)(Q₃₈)(Q₃₉), or —P(Q₃₈)(Q₃₉); or
a combination thereof.

Q₁ to Q₉, Q₁₁ to Q₁₉, Q₂₁ to Q₂₉, and Q₃₁ to Q₃₉ as described herein may each independently be hydrogen, deuterium, —F, —Cl, —Br, —I, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₁-C₆₀ alkylthio group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₇-C₆₀ alkyl aryl group, a substituted or unsubstituted C₇-C₆₀ aryl alkyl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted C₂-C₆₀ alkyl heteroaryl group, a substituted or unsubstituted C₂-C₆₀ heteroaryl alkyl group, a substituted or unsubstituted C₁-C₆₀ heteroaryloxy group, a substituted or unsubstituted C₁-C₆₀ heteroarylthio group, a substituted or unsubstituted monovalent non-aromatic condensed polycyclic group, or a substituted or unsubstituted monovalent non-aromatic condensed heteropolycyclic group.

For example, Q₁ to Q₉, Q₁₁ to Q₁₉, Q₂₁ to Q₂₉, and Q₃₁ to Q₃₉ as described herein may each independently be:
—CH₃, —CD₃, —CD₂H, —CDH₂, —CH₂CH₃, —CH₂CD₃, —CH₂CD₂H, —CH₂CDH₂, —CHDCH₃, —CHDCD₂H, —CHDCDH₂, —CHDCD₃, —CD₂CD₃, —CD₂CD₂H, or —CD₂CDH₂, or
an n-propyl group, an isopropyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, a phenyl group, a biphenyl group, or a naphthyl group, each unsubstituted or substituted with at least one of deuterium, a C₁-C₁₀ alkyl group, a phenyl group, or a combination thereof.

The term “room temperature” as used herein refers to a temperature of about 25° C.

The terms “a biphenyl group, a terphenyl group, and a tetraphenyl group” as used herein each refer to a monovalent group having two, three, or four phenyl groups linked via a single bond, respectively.

Hereinafter, a compound and an organic light-emitting device according to exemplary embodiments are described in further detail with reference to Synthesis Examples and Examples. However, the organic light-emitting device is not limited thereto. The wording “‘B’ was used instead of ‘A’” used in describing Synthesis Examples means that an amount of ‘A’ used was identical to an amount of ‘B’ used, in terms of a molar equivalent.

### EXAMPLES

### Synthesis Example 1: Synthesis of Compound 331

Compound 331 was synthesized according to the following reaction scheme:

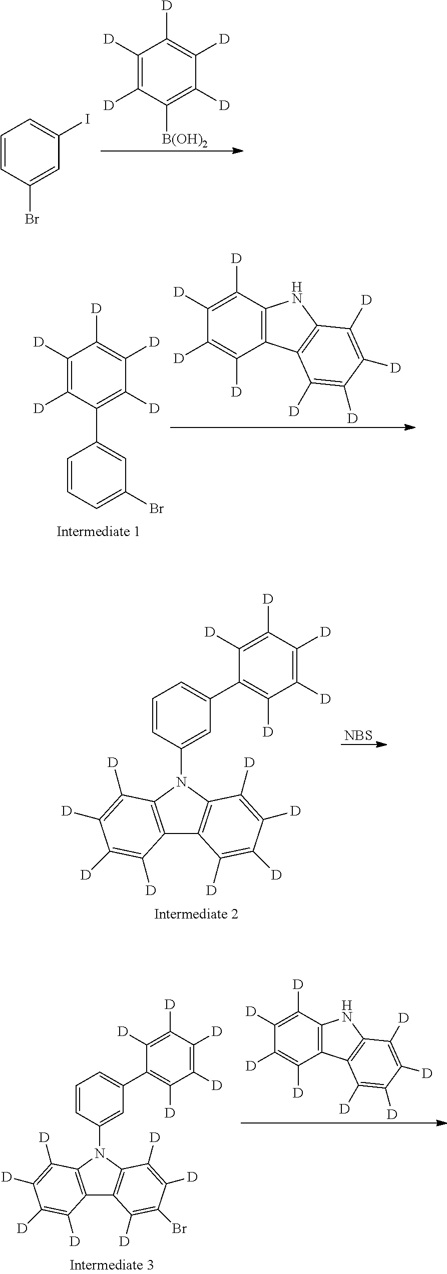

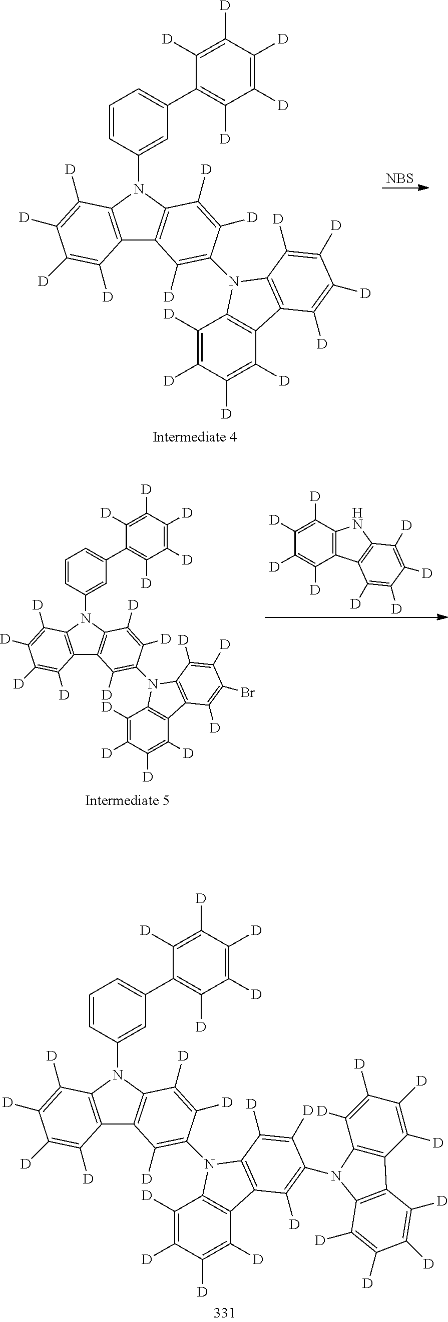

### (1) Synthesis of Intermediate 1

1-bromo-3-iodobenzene (35 grams (g), 123.7 millimoles (mmol)), (phenyl-d₅)boronic acid (13 g, 103 mmol), tetrakis(triphenylphosphine)palladium(0) (5.95 g, 5.15 mmol), and sodium bicarbonate (21.6 g, 257 mmol), were mixed with 400 milliliters (mL) of toluene, 100 mL of ethanol, and 100 mL of deionized (DI) water, and the mixed solution was heated at 120° C. for 16 hours. After completion of the reaction, the reaction solution was cooled to room temperature, and an organic layer was obtained by extraction using ethyl acetate, dried using anhydrous magnesium sulfate (MgSO₄), concentrated under reduced pressure, and then purified by a silica column, to synthesize Intermediate 1. (28.3 g, 118.8 mmol, yield of 96%). The product was identified by liquid chromatography-mass spectrometry (LC-MS).

LC-MS: calculated: 237.02, found (M+1): m/z=238.04.

### (2) Synthesis of Intermediate 2

Intermediate 1 (28.1 g, 118.8 mmol), 9H-carbazole-1,2,3,4,5,6,7,8-d8 (20.8 g, 118.8 mmol), sodium tert-butoxide (17.1 g, 178.3 mmol), tris(dibenzylideneacetone)dipalladium(0) (5.4 g, 5.9 mmol), and tri-tert-butylphosphine (4.8 mL, 11.9 mmol) were mixed with 550 mL of toluene, and the mixed solution was heated at 130° C. for 16 hours. After completion of the reaction, the reaction solution was cooled to room temperature, and an organic layer was obtained by extraction using ethyl acetate, dried using anhydrous MgSO₄, concentrated under reduced pressure, and then purified by a silica column, to synthesize Intermediate 2 (19.5 g, 60.5 mmol, yield of 51%). The product was identified by LC-MS.

LC-MS: calculated: 332.22, found (M+1): m/z=333.25.

### (3) Synthesis of Intermediate 3

Intermediate 2 (20.1 g, 60.5 mmol) was mixed with 600 mL of dimethyl formamide (DMF), and the mixed solution was stirred at 0° C. While maintaining the temperature at 0° C., N-bromosuccinimide (10.2 g, 57.5 mmol) dissolved in 50 mL of DMF was added dropwise thereto and stirred at room temperature for 16 hours. After completion of the reaction, an organic layer was obtained by extraction using ethyl acetate, dried using anhydrous MgSO₄, concentrated under reduced pressure, and then purified by a silica column, to synthesize Intermediate 3 (23.2 g, 56.6 mmol, yield of 93%). The product was identified by LC-MS.

LC-MS: calculated: 409.12, found (M+1): m/z=410.13.

### (4) Synthesis of Intermediate 4

Intermediate 3 (23.2 g, 56.6 mmol), 9H-carbazole-1,2,3,4,5,6,7,8-d8 (10.4 g, 59.4 mmol), sodium tert-butoxide (8.2 g, 84.9 mmol), tris(dibenzylideneacetone)dipalladium(0) (2.1 g, 2.3 mmol), and tri-tert-butylphosphine (1.8 mL, 4.5 mmol) were mixed with 280 mL of toluene, and the mixed solution was heated at 130° C. for 16 hours. After completion of the reaction, the reaction solution was cooled to room temperature, and an organic layer was obtained by extraction using ethyl acetate, dried using anhydrous MgSO₄, concentrated under reduced pressure, and then purified by a silica column, to synthesize Intermediate 4 (19.5 g, 38.6 mmol, yield of 68%). The product was identified by LC-MS.

LC-MS: calculated: 504.32, found (M+1): m/z=505.33.

### (5) Synthesis of Intermediate 5

Intermediate 5 was synthesized in a similar manner as used to synthesize Intermediate 3, except that Intermediate 4 was used instead of Intermediate 2 (yield of 72%). The product was identified by LC-MS.

LC-MS: calculated: 581.22, found (M+1): m/z=582.23.

### (6) Synthesis of Compound 331

Compound 331 was synthesized in a similar manner as used to synthesize Intermediate 4, except that Intermediate 5 was used instead of Intermediate 3 (yield of 52%). The product was identified by LC-MS.

LC-MS: calculated: 676.42, found (M+1): m/z=677.45.

### Synthesis Example 2: Synthesis of Compound 339

Compound 339 was synthesized according to the following reaction scheme:

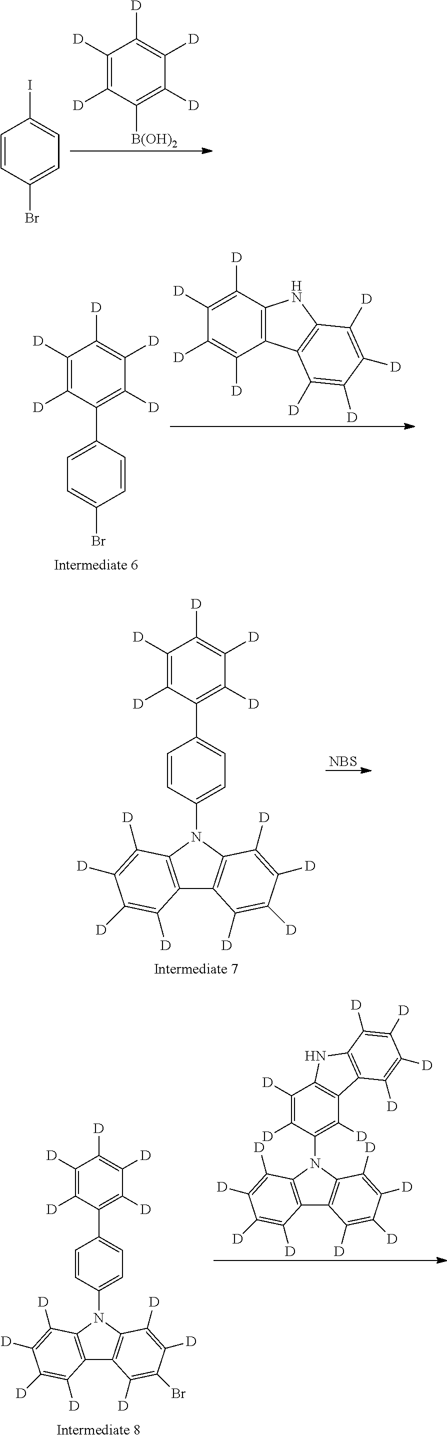

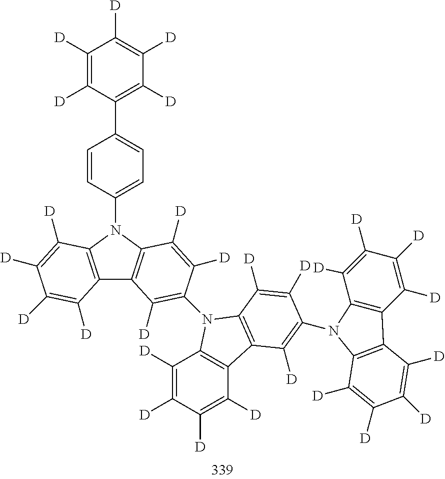 [SMILES: [2H]c1c([2H])c([2H])c(-c2ccc(-n3c4c([2H])c([2H])c([2H])c([2H])c4c4c([2H])c(-n5c6c([2H])c([2H])c([2H])c([2H])c6c6c([2H])c(-n7c8c([2H])c([2H])c([2H])c([2H])c8c8c([2H])c([2H])c([2H])c([2H])c87)c([2H])c([2H])c65)c([2H])c([2H])c43)cc2)c([2H])c1[2H]]

### (1) Synthesis of Intermediate 6

Intermediate 6 was synthesized (yield of 85%) in a similar manner as used to synthesize Intermediate 1 of Synthesis Example 1, except that 1-bromo-4-iodobenzene was used instead of 1-bromo-3-iodobenzene. The product was identified by LC-MS.

LC-MS: calculated: 237.02, found (M+1): m/z=238.04.

### (2) Synthesis of Compound 339

Compound 339 was synthesized in a similar manner as used to synthesize Intermediate 4 of Synthesis Example 1, except that Intermediate 6 was used instead of Intermediate 1, Intermediate 7 was used instead of Intermediate 2, Intermediate 7 was used instead of Intermediate 3, and 9H-3,9′-bicarbazole-1,1′,2,2′,3′,4,4′,5,5′,6,6′,7,7′,8,8′-d₁₅ was used instead of 9H-carbazole-1,2,3,4,5,6,7,8-d₈. The product was identified by LC-MS.

LC-MS: calculated: 676.42, found (M+1): m/z=677.45.

### Synthesis Example 3: Synthesis of Compound 183

Compound 183 was synthesized according to the following reaction scheme:

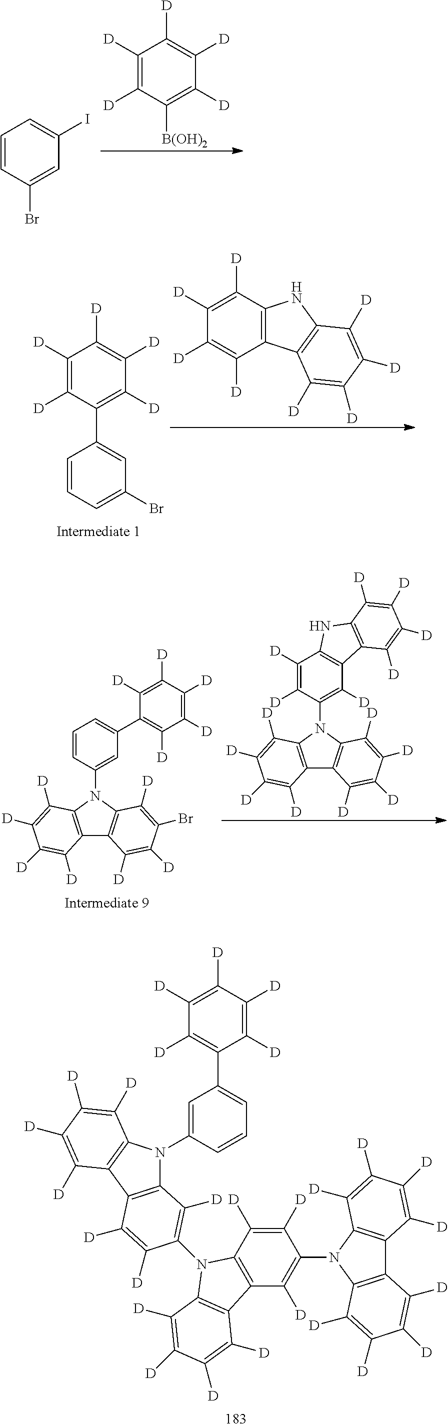

### (1) Synthesis of Intermediate 9

Intermediate 9 was synthesized (yield of 55%) in a similar manner as used to synthesize Intermediate 2 of Synthesis Example 1, except that 2-bromo-9H-carbazole-1,3,4,5,6,7,8-d₇ was used instead of 9H-carbazole-1,2,3,4,5,6,7,8-d₈. The product was identified by LC-MS.

LC-MS: calculated: 409.12, found (M+1): m/z=410.13.

### (2) Synthesis of Compound 183

Compound 183 was synthesized in a similar manner as used to synthesize Intermediate 4 of Synthesis Example 1, except that Intermediate 9 was used instead of Intermediate 3 and 9H-3,9′-bicarbazole-1,1′,2,2′,3′,4,4′,5,5′,6,6′,7,7′,8,8′-d₁₅ was used instead of H-carbazole-1,2,3,4,5,6,7,8-d₈. The product was identified by LC-MS.

LC-MS: calculated: 676.42, found (M+1): m/z=677.45.

### Example 1

A glass substrate on which a 1,500 Å-thick indium tin oxide (ITO) electrode (first electrode, anode) was formed was cleaned by ultrasonication using DI water. After the completion of ultrasonication using the DI water, cleaning by ultrasonication using a solvent, including each of isopropyl alcohol, acetone, and methanol, was performed, and the glass substrate was dried and transferred to a plasma cleaner. The glass substrate was cleaned by using oxygen plasma for 5 minutes, and then transferred to a vacuum laminator.

Compound HT3 and Compound HT-D2 were co-deposited by vacuum on the ITO electrode on the glass substrate to form a hole injection layer having a thickness of 100 Å, Compound HT3 was deposited by vacuum on the hole injection layer to form a hole transport layer having a thickness of 1,300 Å, and mCP was deposited by vacuum on the hole transport layer to form an electron blocking layer having a thickness of 100 Å, so as to form a hole transport region.

On the hole transport region, a host and Compound P31 as a dopant were co-deposited by vacuum at a weight ratio of 85:15 to form an emission layer having a thickness of 300 Å. For the host, a first host (Compound 331) and a second host (Compound E1) were used, and a weight ratio thereof was adjusted to 6:4.

BCP was vacuum-deposited on the emission layer to form a hole blocking layer having a thickness of 100 Å, Compound ET3 and LiQ were co-deposited by vacuum on the hole blocking layer to form an electron transport layer having a thickness of 300 Å, LiQ was deposited by vacuum on the electron transport layer to form an electron injection layer having a thickness of 10 Å, and an Al second electrode (cathode) having a thickness of 1,200 Å was formed on the electron injection layer, thereby completing the manufacture of an organic light-emitting device.

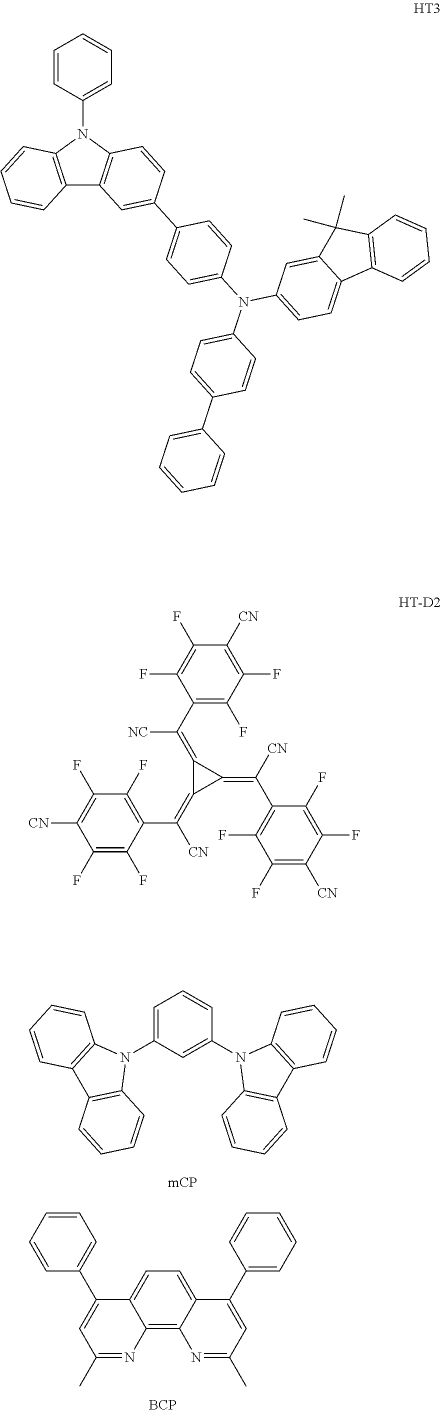

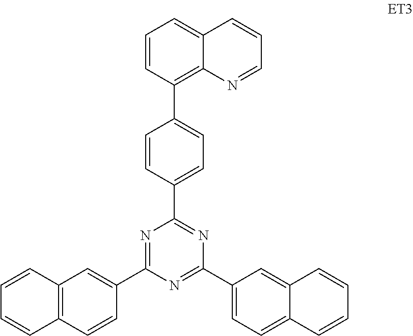 [SMILES: c1ccc2cc(-c3nc(-c4ccc(-c5cccc6cccnc56)cc4)nc(-c4ccc5ccccc5c4)n3)ccc2c1]

### Examples 2 and 3 and Comparative Examples 1 to 3

Organic light-emitting devices were manufactured in a similar manner as in Example 1, except that compounds shown in Table 2 were each used instead of Compound 331 in forming the emission layer.

### Evaluation Example 1: Characterization of Organic Light-Emitting Device

For each of the organic light-emitting devices of Examples 1 to 3 and Comparative Examples 1 to 3, the lifespan (T₉₅ at 1,000 cd/A², relative %) was evaluated with a current-voltage meter (Keithley 2400) and a luminance meter (Minolta Cs-1000A), and results thereof are shown in Table 2. Here, the lifespan (T₉₅) was a measure of the time required for the luminance to decline to 95% of the initial luminance of 100%. The T₉₅ lifespans of the organic light-emitting devices of Examples 1 to 3 were represented as relative values (%) compared to the T₉₅ lifespans of the organic light-emitting devices of Comparative Examples 1 to 3, respectively.

TABLE 2

| | Host in | | | |
|---|---|---|---|---|
| | emission | | | |
| | layer | | | |
| | First | Second | | Lifespan |
| | host | host | Dopant | (T₉₅, relative %) |
| Example 1 | 331 | E1 | P31 | 178 |
| | | | | (compared to |
| | | | | Comparative Example 1) |
| Comparative | CE1 | E1 | P31 | 100 |
| Example 1 | | | | |
| Example 2 | 339 | E1 | P31 | 157 |
| | | | | (compared to |
| | | | | Comparative Example 2) |
| Comparative | CE2 | E1 | P31 | 100 |
| Example 2 | | | | |
| Example 3 | 183 | E1 | P31 | 188 |
| | | | | (compared to |
| | | | | Comparative Example 3) |
| Comparative | CE3 | E1 | P31 | 100 |
| Example 3 | | | | |

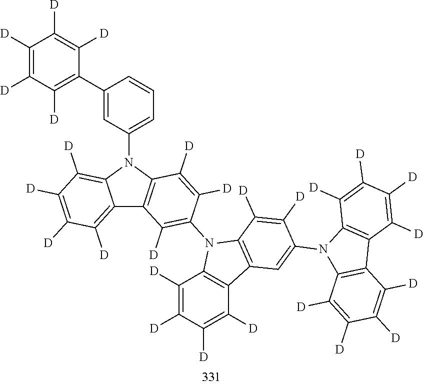 [SMILES: [2H]c1c([2H])c([2H])c(-c2cccc(-n3c4c([2H])c([2H])c([2H])c([2H])c4c4c([2H])c(-n5c6c([2H])c([2H])c(-n7c8c([2H])c([2H])c([2H])c([2H])c8c8c([2H])c([2H])c([2H])c([2H])c87)cc6c6c([2H])c([2H])c([2H])c([2H])c65)c([2H])c([2H])c43)c2)c([2H])c1[2H]]

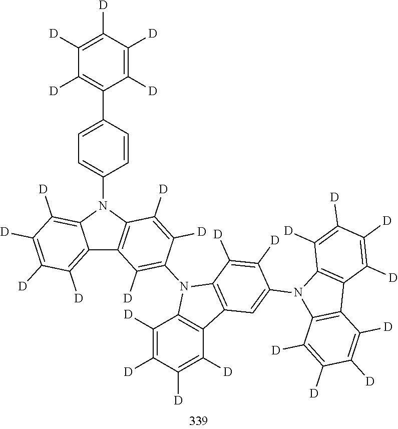 [SMILES: [2H]c1c([2H])c([2H])c(-c2ccc(-n3c4c([2H])c([2H])c([2H])c([2H])c4c4c([2H])c(-n5c6c([2H])c([2H])c(-n7c8c([2H])c([2H])c([2H])c([2H])c8c8c([2H])c([2H])c([2H])c([2H])c87)cc6c6c([2H])c([2H])c([2H])c([2H])c65)c([2H])c([2H])c43)cc2)c([2H])c1[2H]]

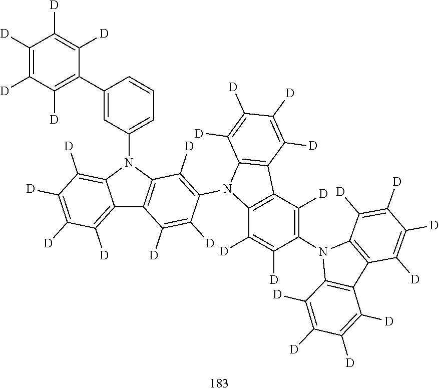 [SMILES: [2H]c1c([2H])c([2H])c(-c2cccc(-n3c4c([2H])c([2H])c([2H])c([2H])c4c4c([2H])c([2H])c(-n5c6c([2H])c([2H])c([2H])c([2H])c6c6c([2H])c(-n7c8c([2H])c([2H])c([2H])c([2H])c8c8c([2H])c([2H])c([2H])c([2H])c87)c([2H])c([2H])c65)c([2H])c43)c2)c([2H])c1[2H]]

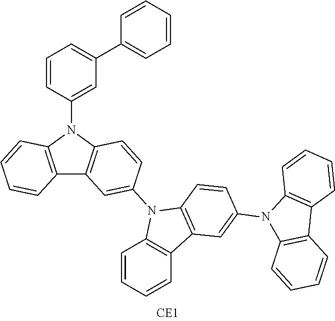 [SMILES: c1ccc(-c2cccc(-n3c4ccccc4c4cc(-n5c6ccccc6c6cc(-n7c8ccccc8c8ccccc87)ccc65)ccc43)c2)cc1]

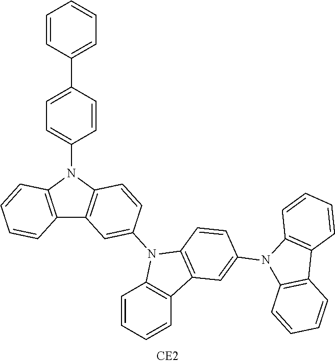 [SMILES: c1ccc(-c2ccc(-n3c4ccccc4c4cc(-n5c6ccccc6c6cc(-n7c8ccccc8c8ccccc87)ccc65)ccc43)cc2)cc1]

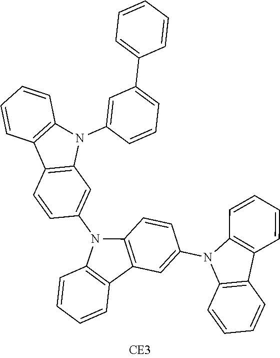 [SMILES: c1ccc(-c2cccc(-n3c4ccccc4c4ccc(-n5c6ccccc6c6cc(-n7c8ccccc8c8ccccc87)ccc65)cc43)c2)cc1]

Referring to Table 2, it was confirmed that the organic light-emitting device of Example 1 had a T₉₅ lifespan improvement of up to 178% compared to the organic light-emitting device of Comparative Example 1, the organic light-emitting device of Example 2 had a T₉₅ lifespan improvement of up to 157% compared to the organic light-emitting device of Comparative Example 2, and the organic light-emitting device of Example 3 had a T₉₅ lifespan improvement of up to 188% compared to the organic light-emitting device of Comparative Example 3.

### Example 4

A glass substrate on which a 1,500 Å-thick indium tin oxide (ITO) electrode (first electrode, anode) was formed was cleaned by ultrasonication using DI water. After the completion of ultrasonication using DI water, cleaning by ultrasonication using a solvent, including isopropyl alcohol, acetone, and methanol, was performed, and the glass substrate was dried and transferred to a plasma cleaner. The glass substrate was cleaned by using oxygen plasma for 5 minutes, and then transferred to a vacuum laminator.

Compound HT3 and Compound HT-D2 were co-deposited by vacuum on the ITO electrode on the glass substrate to form a hole injection layer having a thickness of 100 Å, Compound HT3 was deposited by vacuum on the hole injection layer to form a hole transport layer having a thickness of 1,300 Å, and mCP was deposited by vacuum on the hole transport layer to form an electron blocking layer having a thickness of 100 Å, so as to form a hole transport region.

On the hole transport region, a host, a phosphorescent dopant (Compound P31), and a fluorescent dopant (Compound D3) were co-deposited by vacuum at a weight ratio of 85:14:1 to form an emission layer having a thickness of 300 Å. For the host, a first host (Compound 331) and a second host (Compound E1) were used, and a weight ratio thereof was adjusted to 6:4.

BCP was vacuum-deposited on the emission layer to form a hole blocking layer having a thickness of 100 Å, Compound ET3 and LiQ were co-deposited by vacuum on the hole blocking layer to form an electron transport layer having a thickness of 300 Å, LiQ was deposited by vacuum on the electron transport layer to form an electron injection layer having a thickness of 10 Å, and an Al second electrode (cathode) having a thickness of 1,200 Å was formed on the electron injection layer, thereby completing the manufacture of an organic light-emitting device.

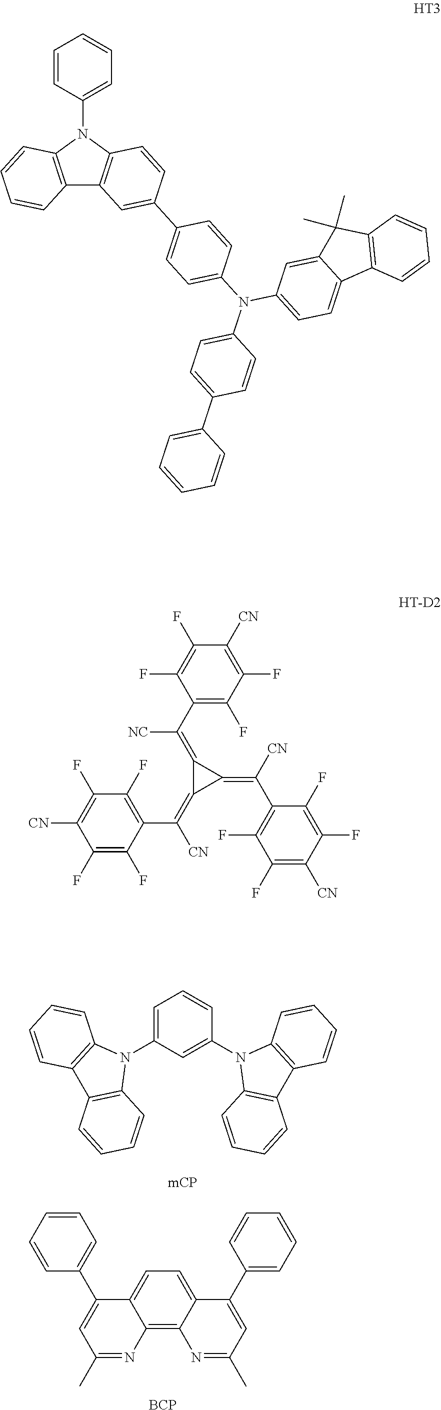

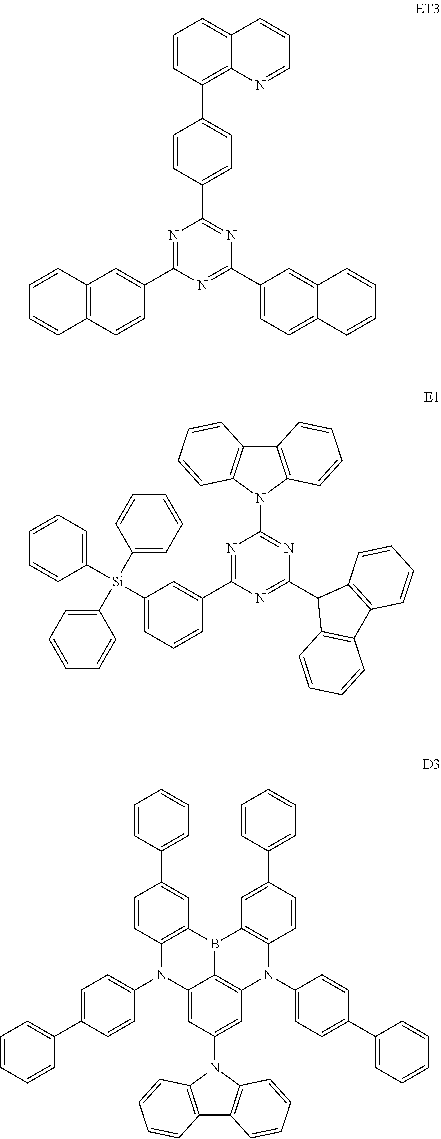 [SMILES: c1ccc2cc(-c3nc(-c4ccc(-c5cccc6cccnc56)cc4)nc(-c4ccc5ccccc5c4)n3)ccc2c1 ; c1ccc([Si](c2ccccc2)(c2ccccc2)c2cccc(-c3nc(C4c5ccccc5-c5ccccc54)nc(-n4c5ccccc5c5ccccc54)n3)c2)cc1 ; c1ccc(-c2ccc(N3c4ccc(-c5ccccc5)cc4B4c5cc(-c6ccccc6)ccc5N(c5ccc(-c6ccccc6)cc5)c5cc(-n6c7ccccc7c7ccccc76)cc3c54)cc2)cc1]

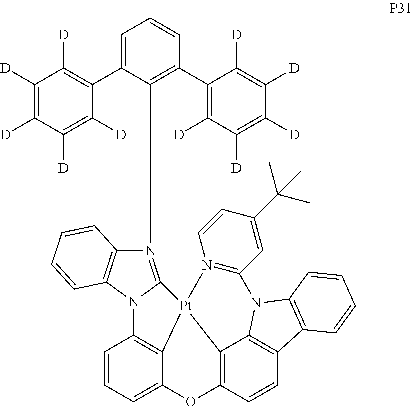

### Examples 5 and 6 and Comparative Examples 4 to 6

Organic light-emitting devices were manufactured in a similar manner as in Example 2, except that compounds shown in Table 3 were each used instead of Compound 331 in forming the emission layer.

### Evaluation Example 2: Characterization of Organic Light-Emitting Devices

For each of the organic light-emitting devices of Examples 4 to 6 and Comparative Examples 4 to 6, the lifespan (T₉₅ at 1,000 cd/cm², hours) was evaluated with a current-voltage meter (Keithley 2400) and a luminance meter (Minolta Cs-1000A), and results thereof are shown in Table 3. Here, the lifespan (T₉₅, relative %) was a measure of the time required for the luminance to decline to 95% of the initial luminance of 100%. The T₉₅ lifespans of the organic light-emitting devices of Examples 4 to 6 were represented as relative values (%) compared to the T₉₅ lifespans of the organic light-emitting devices of Comparative Examples 4 to 6, respectively.

TABLE 3

| | | | Dopant in | | |
|---|---|---|---|---|---|
| | Host in | | emission layer | | |
| | emission layer | | Phospho- | | Lifespan |
| | First | Second | rescent | Fluorescent | (T₉₅, |
| | host | host | dopant | dopant | relative %) |
| Example 4 | 331 | E1 | P31 | D3 | 158 |
| | | | | | (compared to |
| | | | | | Comparative |
| | | | | | Example 4) |
| Comparative | CE1 | E1 | P31 | D3 | 100 |
| Example 4 | | | | | |
| Example 5 | 339 | E1 | P31 | D3 | 158 |
| | | | | | (compared to |
| | | | | | Comparative |
| | | | | | Example 5) |
| Comparative | CE2 | E1 | P31 | D3 | 100 |
| Example 5 | | | | | |
| Example 6 | 183 | E1 | P31 | D3 | 149 |
| | | | | | (compared to |
| | | | | | Comparative |
| | | | | | Example 6) |
| Comparative | CE3 | E1 | P31 | D3 | 100 |
| Example 6 | | | | | |

Referring to Table 3, it was confirmed that the organic light-emitting device of Example 4 had a T₉₅ lifespan improvement of up to 158% compared to the organic light-emitting device of Comparative Example 4, the organic light-emitting device of Example 5 had a T₉₅ lifespan improvement of up to 158% compared to the organic light-emitting device of Comparative Example 5, and the organic light-emitting device of Example 6 had a T₉₅ lifespan improvement of up to 149% compared to the organic light-emitting device of Comparative Example 6.

According to the one or more exemplary embodiments, use of at least one of the heterocyclic compounds represented by Formula 1 may provide an organic light-emitting device having long lifespan characteristics, and an electronic apparatus including the organic light-emitting device may also have long lifespan characteristics.

It should be understood that the exemplary embodiments described herein should be considered in a descriptive sense only and not for purposes of limitation. Descriptions of features or aspects within each exemplary embodiment should typically be considered as available for other similar features or aspects in other embodiments. While one or more exemplary embodiments have been described with reference to the FIGURE, it will be understood by those of ordinary skill in the art that various changes in form and details may be made therein without departing from the spirit and scope as defined by the following claims.

What is claimed is:

1. A heterocyclic compound represented by Formula 1: 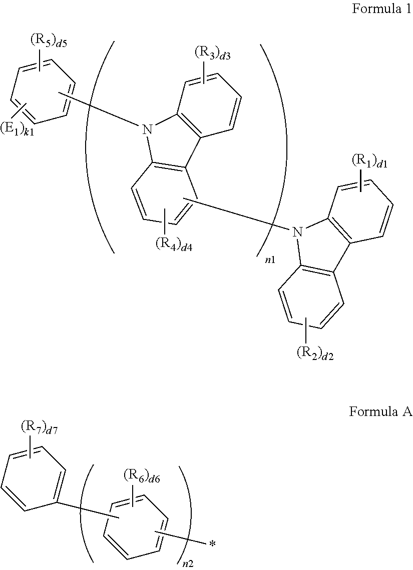
wherein, in Formulae 1 and A,
E₁ is a group represented by Formula A,
k1 is an integer from 1 to 5,
n1 is 2 or 3,
n2 is an integer from 0 to 3,
R₁ to R₆ are each independently hydrogen, deuterium, —F, —Cl, —Br, —I, —SF₅, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-Coo alkynyl group, a substituted or unsubstituted C₁-C₆₀ alkoxy group, a substituted or unsubstituted C₁-C₆₀ alkylthio group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a substituted or unsubstituted C₆-C₆₀ aryl group, a substituted or unsubstituted C₇-C₆₀ alkyl aryl group, a substituted or unsubstituted C₇-C₆₀ aryl alkyl group, a substituted or unsubstituted C₆-C₆₀ aryloxy group, a substituted or unsubstituted C₆-C₆₀ arylthio group, a substituted or unsubstituted C₁-C₆₀ heteroaryl group, a substituted or unsubstituted C₂-C₆₀ alkyl heteroaryl group, a substituted or unsubstituted C₂-C₆₀ heteroaryl alkyl group, a substituted or unsubstituted C₁-C₆₀ heteroaryloxy group, a substituted or unsubstituted C₁-C₆₀ heteroarylthio group, a substituted or unsubstituted monovalent non-aromatic condensed polycyclic group, a substituted or unsubstituted monovalent non-aromatic condensed heteropolycyclic group, —N(Q₁)(Q₂), —Si(Q₃)(Q₄)(Q₅), —Ge(Q₃)(Q₄)(Q₅), —B(Q₆)(Q₇), —P(═O)(Q₈)(Q₉), or —P(Q₈)(Q₉),
R₇ is hydrogen, deuterium, —F, —Cl, —Br, —I, —SF₅, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₁-C₆₀ alkylthio group, a substituted or unsubstituted C₃-C₁₀ cycloalkyl group, a substituted or unsubstituted C₁-C₁₀ heterocycloalkyl group, a substituted or unsubstituted C₃-C₁₀cycloalkenyl group, a substituted or unsubstituted C₁-C₁₀ heterocycloalkenyl group, a substituted or unsubstituted C₆-C₆₀ aryloxy group, a substituted or unsubstituted C₆-C₆₀ arylthio group, a substituted or unsubstituted C₁-C₆₀ heteroaryloxy group, a substituted or unsubstituted C₁-C₆₀ heteroarylthio group, —N(Q₁)(Q₂), —Si(Q₃)(Q₄)(Q₅), —Ge(Q₃)(Q₄)(Q₅), —B(Q₆)(Q₇), —P(═O)(Q₈)(Q₉), or —P(Q₈)(Q₉),
d1, d2, d3, d5, and d6 are each independently an integer from 0 to 4,
d4 is an integer from 0 to 3,
d7 is an integer from 0 to 5,
the heterocyclic compound represented by Formula 1 comprises at least one deuterium,
* indicates a binding site to an adjacent atom, and
at least one substituent of the substituted C₁-C₆₀ alkyl group, the substituted C₂-C₆₀ alkenyl group, the substituted C₂-C₆₀ alkynyl group, the substituted C₁-C₆₀ alkoxy group, the substituted C₁-C₆₀ alkylthio group, the substituted C₃-C₁₀ cycloalkyl group, the substituted C₁-C₁₀heterocycloalkyl group, the substituted C₃-C₁₀ cycloalkenyl group, the substituted C₁-C₁₀ heterocycloalkenyl group, the substituted C₆-C₆₀ aryl group, the substituted C₁-C₆₀ alkyl aryl group, the substituted C₇-C₆₀ aryl alkyl group, the substituted C₆-C₆₀ aryloxy group, the substituted C₆-C₆₀ arylthio group, the substituted C₁-C₆₀ heteroaryl group, the substituted C₂-C₆₀ alkyl heteroaryl group, the substituted C₁-C₆₀ heteroaryl alkyl group, the substituted C₁-C₆₀ heteroaryloxy group, the substituted C₁-C₆₀ heteroarylthio group, the substituted monovalent non-aromatic condensed polycyclic group, and the substituted monovalent non-aromatic condensed heteropolycyclic group is:
deuterium, —F, —Cl, —Br, —I, —SF₅, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, or a C₁-C₆₀ alkylthio group;
a C₁-C₆₀ alkyl group, a C₂-C₆₀ alkenyl group, a C₂-C₆₀ alkynyl group, a C₁-C₆₀ alkoxy group, or a C₁-C₆₀ alkylthio group, each substituted with at least one of deuterium, —F, —Cl, —Br, —I, —SF₅, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₇-C₆₀ alkyl aryl group, a C₆-C₆₀ aryloxy group, a C₆-C₆₀ arylthio group, a C₁-C₆₀ heteroaryl group, a C₂-C₆₀ alkyl heteroaryl group, a C₁-C₆₀ heteroaryloxy group, a C₁-C₆₀ heteroarylthio group, a monovalent non-aromatic condensed polycyclic group, a monovalent non-aromatic condensed heteropolycyclic group, —N(Q₁₁)(Q₁₂), —Si(Q₁₁)(Q₁₂)(Q₁₃), —Ge(Q₁₃)(Q₁₄)(Q₁₅), —B(Q₁₆)(Q₁₇), —P(═O)(Q₁₈)(Q₁₉), —P(Q₁₈)(Q₁₉), or a combination thereof;
a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₇-C₆₀ alkyl aryl group, a C₆-C₆₀ aryloxy group, a C₆-C₆₀ arylthio group, a C₁-C₆₀ heteroaryl group, a C₂-C₆₀ alkyl heteroaryl group, a C₁-C₆₀ heteroaryloxy group, a C₁-C₆₀ heteroarylthio group, a monovalent non-aromatic condensed polycyclic group, or a monovalent non-aromatic condensed heteropolycyclic group, each unsubstituted or substituted with at least one of deuterium, —F, —Cl, —Br, —I, —SF₅, —CD₃, —CD₂H, —CDH₂, —CF₃, —CF₂H, —CFH₂, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a C₁-C₆₀ alkyl group, a C₂-C₆₀ alkenyl group, a C₂-C₆₀ alkynyl group, a C₁-C₆₀ alkoxy group, a C₁-C₆₀ alkylthio group, a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₇-C₆₀ alkyl aryl group, a C₇-C₆₀ aryl alkyl group, a C₆-C₆₀ aryloxy group, a C₆-C₆₀ arylthio group, a C₁-C₆₀ heteroaryl group, a C₂-C₆₀ alkyl heteroaryl group, a C₂-C₆₀ heteroaryl alkyl group, a C₁-C₆₀ heteroaryloxy group, a C₁-C₆₀ heteroarylthio group, a monovalent non-aromatic condensed polycyclic group, a monovalent non-aromatic condensed heteropolycyclic group, —N(Q₂₁)(Q₂₂), —Si(Q₂₃)(Q₂₄)(Q₂₅), —Ge(Q₂₃)(Q₂₄)(Q₂₅), —B(Q₂₆)(Q₂₇), —P(═O)(Q₂₈)(Q₂₉), —P(Q₂₈)(Q₂₉) or a combination thereof;
—N(Q₃₁)(Q₃₂), —Si(Q₃₃)(Q₃₄)(Q₃₅), —Ge(Q₃₃)(Q₃₄)(Q₃₅), —B(Q₃₆)(Q₃₇), —P(═O)(Q₃₈)(Q₃₉), or —P(Q₃₈)(Q₃₉); or
a combination thereof, and
Q₁ to Q₉, Q₁₁ to Q₁₉, Q₂₁ to Q₂₉, and Q₃₁ to Q₃₉ are each independently hydrogen, deuterium, —F, —Cl, —Br, —I, —SF₅, a hydroxyl group, a cyano group, a nitro group, an amino group, an amidino group, a hydrazine group, a hydrazone group, a carboxylic acid group or a salt thereof, a sulfonic acid group or a salt thereof, a phosphoric acid group or a salt thereof, a substituted or unsubstituted C₁-C₆₀ alkyl group, a substituted or unsubstituted C₂-C₆₀ alkenyl group, a substituted or unsubstituted C₂-C₆₀ alkynyl group, a substituted or unsubstituted C₁-C₆₀ alkoxy group, a substituted or unsubstituted C₁-C₆₀ alkylthio group, a substituted or unsubstituted C₃-C₁₀ cycloalkyl group, a substituted or unsubstituted C₁-C₁₀ heterocycloalkyl group, a substituted or unsubstituted C₃-C₁₀ cycloalkenyl group, a substituted or unsubstituted C₁-C₁₀ heterocycloalkenyl group, a C₆-C₆₀ aryl group, a substituted or unsubstituted C₇-C₆₀ alkyl aryl group, a substituted or unsubstituted C₇-C₆₀ aryl alkyl group, a substituted or unsubstituted C₆-C₆₀ aryloxy group, a substituted or unsubstituted C₆-C₆₀ arylthio group, a C₁-C₆₀ heteroaryl group, a substituted or unsubstituted C₂-C₆₀ alkyl heteroaryl group, a substituted or unsubstituted C₂-C₆₀ heteroaryl alkyl group, a substituted or unsubstituted C₁-C₆₀ heteroaryloxy group, a substituted or unsubstituted C₁-C₆₀ heteroarylthio group, a substituted or unsubstituted monovalent non-aromatic condensed polycyclic group, or a substituted or unsubstituted monovalent non-aromatic condensed heteropolycyclic group.

2. The heterocyclic compound of claim 1, wherein the heterocyclic compound represented by Formula 1 satisfies at least one of Conditions 1 to 7:
Condition 1
at least one R₁ is deuterium;
Condition 2
at least one R₂ is deuterium;
Condition 3
at least one Ra is deuterium;
Condition 4
at least one R₄ is deuterium;
Condition 5
at least one R₅ is deuterium;
Condition 6
at least one Re is deuterium;
Condition 7
at least one R₇ is deuterium.

3. The heterocyclic compound of claim 1, wherein the heterocyclic compound represented by Formula 1 satisfies at least one of Conditions 1′ to 7′:
Condition 1′
each R₁ is deuterium, and
d1 is 4;
Condition 2′
each R₂ is deuterium, and
d2 is 4;
Condition 3′
each R₃ is deuterium, and
d3 is 4;
Condition 4′
each R₄ is deuterium, and
d4 is 3;
Condition 5′
each R₅ is deuterium, and
k1 is 1, and d5 is 4; or k1 is 2, and d5 is 3;
Condition 6′
each Re is deuterium, and
d6 is 4;
Condition 7′
each R₇ is deuterium, and
d7 is 4.

4. The heterocyclic compound of claim 1, wherein a substitution ratio of deuterium of the heterocyclic compound represented by Formula 1 is greater than or equal to 10%.

5. The heterocyclic compound of claim 1, wherein n2 is 0 or 1.

6. The heterocyclic compound of claim 1, wherein a moiety represented by 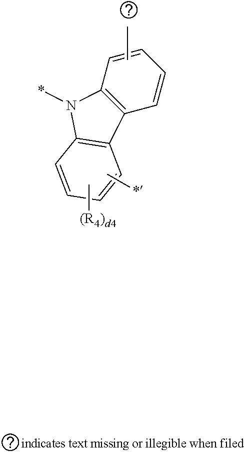
in Formula 1 is a group represented by one of Formulae 2-1 to 2-4: 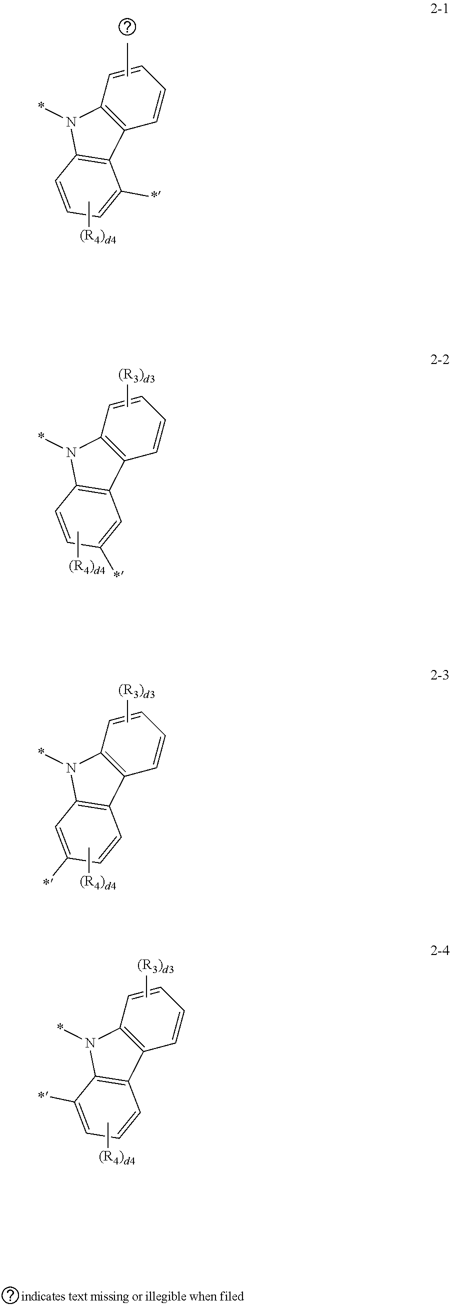
wherein, in Formulae 2-1 to 2-4,
R₃, R₄, d3, and d4 are each as defined in claim 1, and
* and *′ each indicate a binding site to a neighboring atom.

7. The heterocyclic compound of claim 1, wherein a moiety represented by 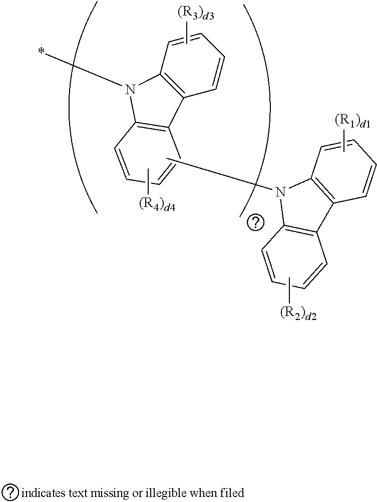
in Formula 1 is a group represented by one of Formulae 3-1 to 3-16: 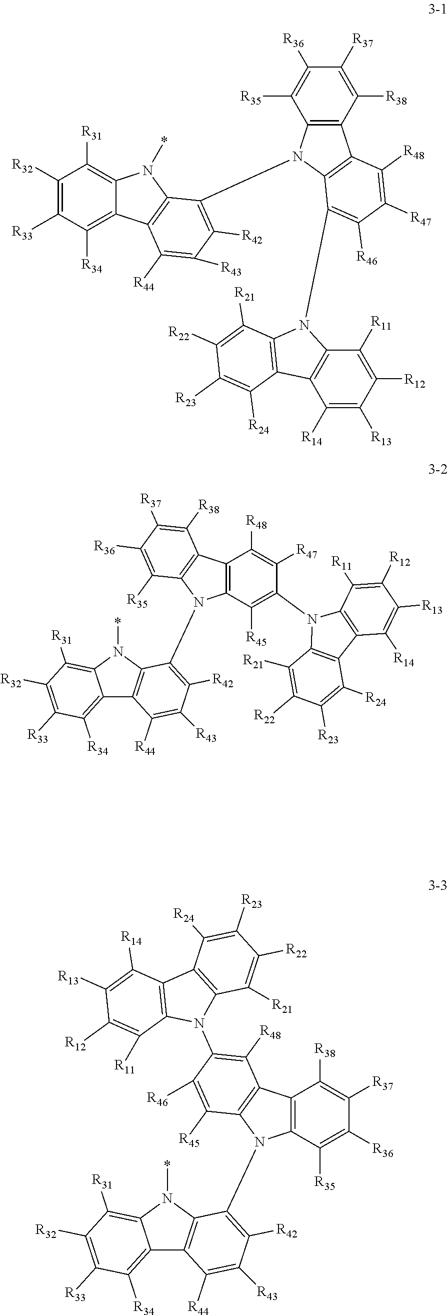 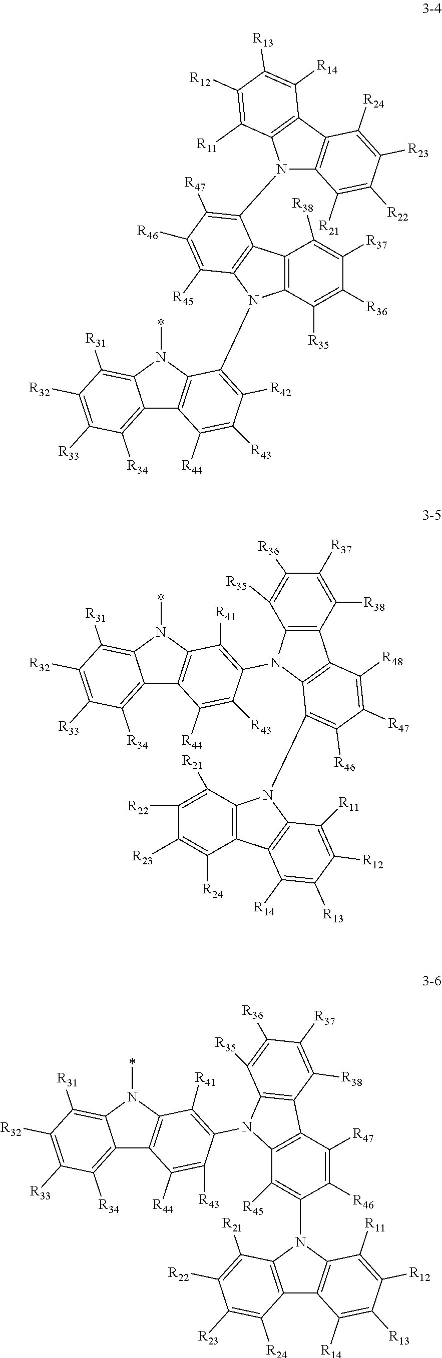 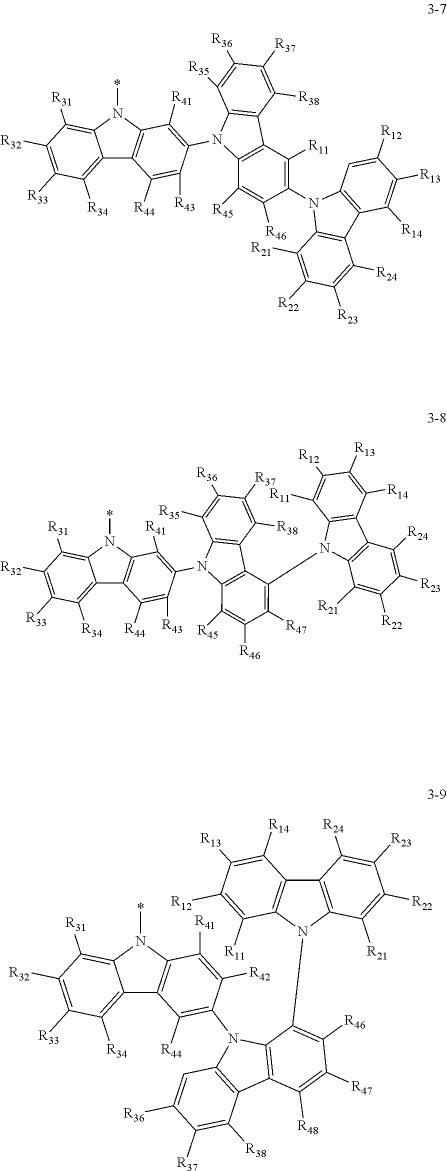 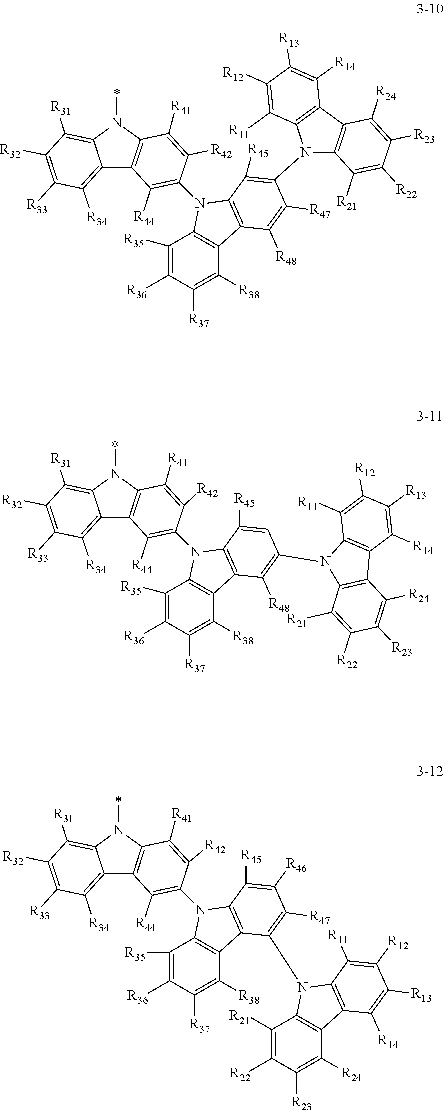 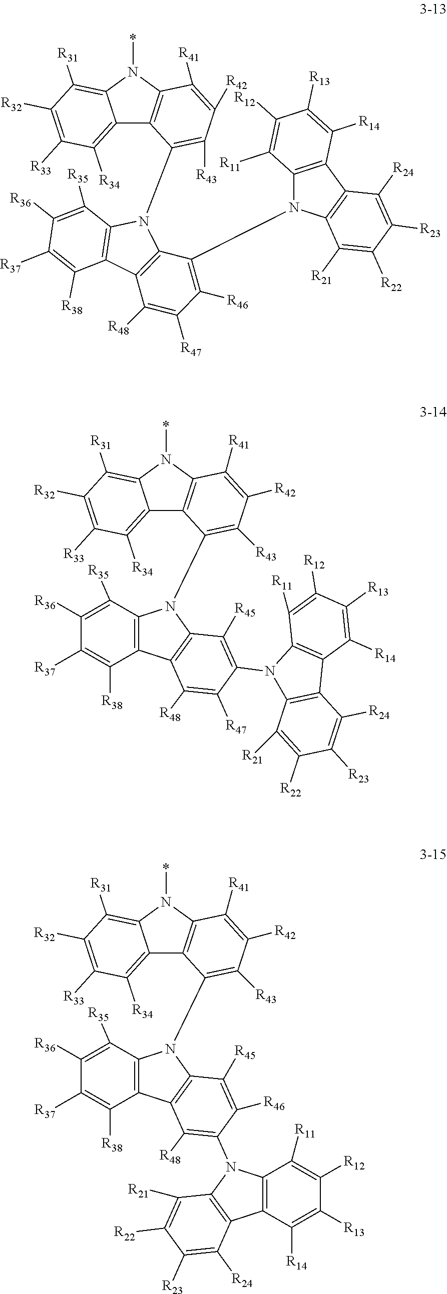 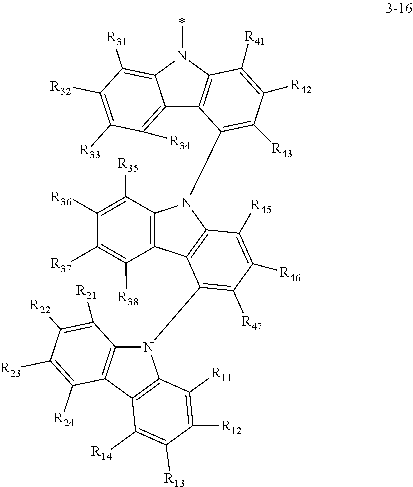
wherein, in Formulae 3-1 to 3-16,
R₁₁ to R₁₄ are each as defined for R₁ in claim 1,
R₂₁ to R₂₄ are each as defined for R₂ in claim 1,
R₃₁ to R₃₈ are each as defined for R₃ in claim 1,
R₄₁ to R₄₈ are each as defined for R₄ in claim 1, and
* indicates a binding site to a neighboring atom.

8. The heterocyclic compound of claim 1, wherein the group represented by Formula A may be represented by one of Formulae A-1 to A-13: 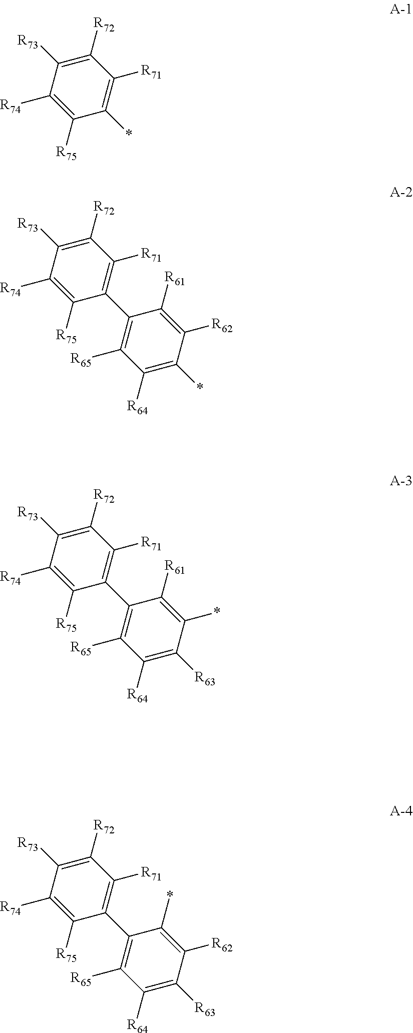 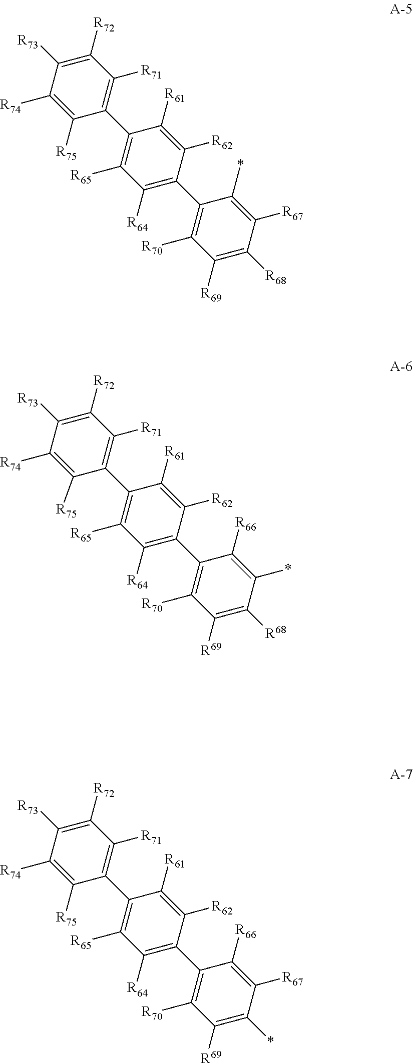 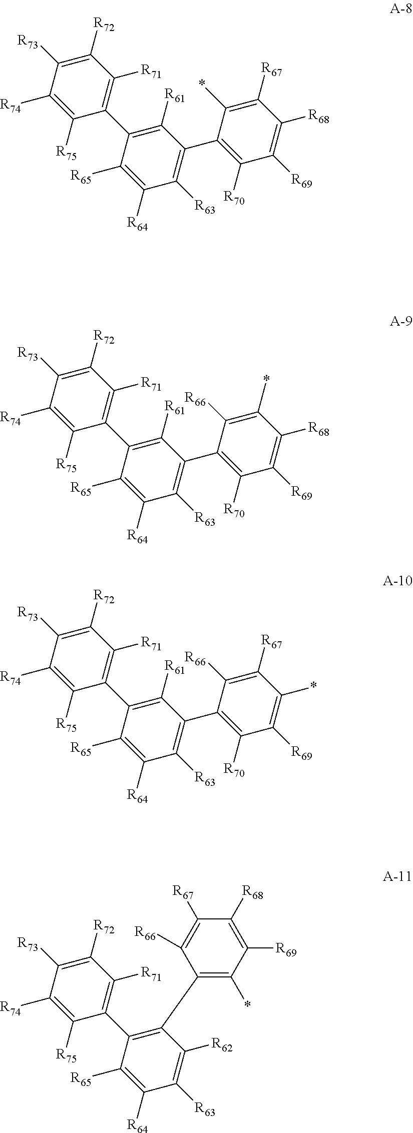 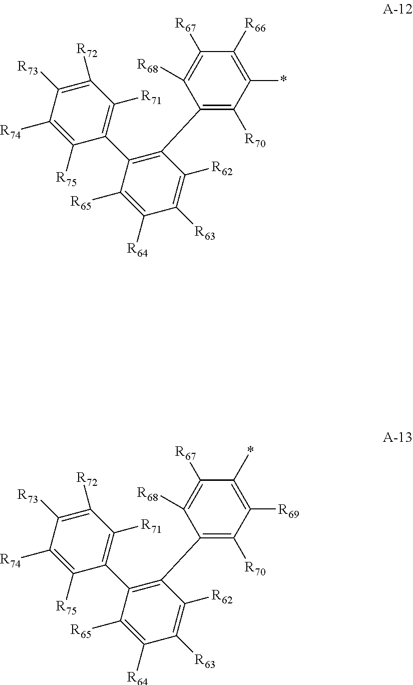
wherein, in Formulae A-1 to A-13,
R₇₁ to R₇₅ are each as defined for R₇ in claim 1,
R₆₁ to R₇₀ are each as defined for R₆ in claim 1,
* indicates a binding site to a neighboring atom.

9. The heterocyclic compound of claim 1, wherein a moiety represented by 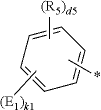
in Formula 1 is a group represented by one of Formulae 4-1 to 4-17: 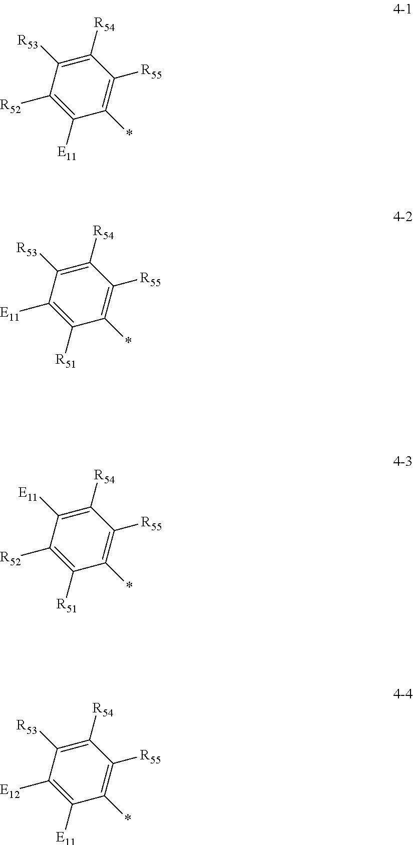 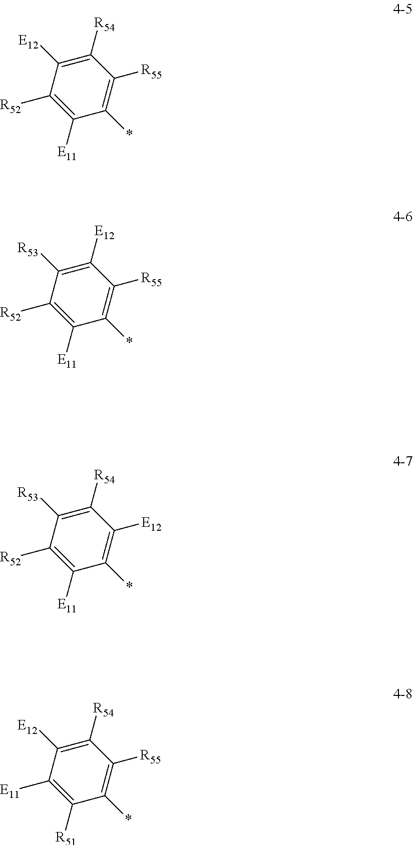 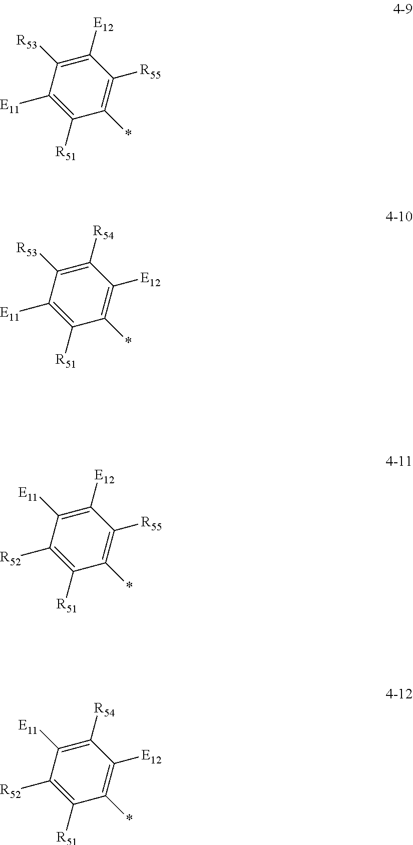 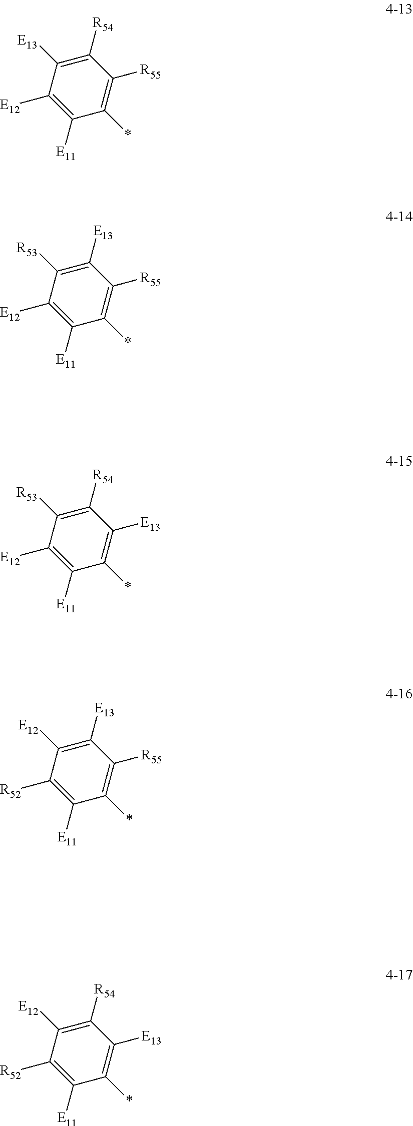
wherein, in Formulae 4-1 to 4-17,
E₁₁ to E₁₃ are each independently as defined for E₁ in claim 1,
R₅₁ to R₅₅ are each independently as defined for R₅ in claim 1, and
* indicates a binding site to a neighboring atom.

10. The heterocyclic compound of claim 1, wherein the heterocyclic compound represented by Formula 1 is represented by one of Formulae 1-1 to 1-36, 1-73 to 1-111, 1-145 to 1-180, or 1-217 to 1-252: 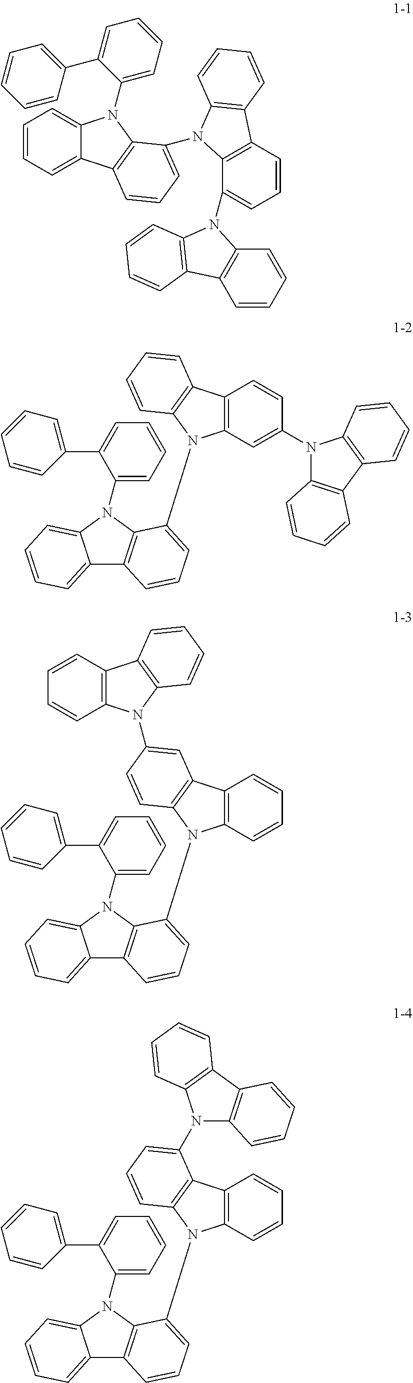 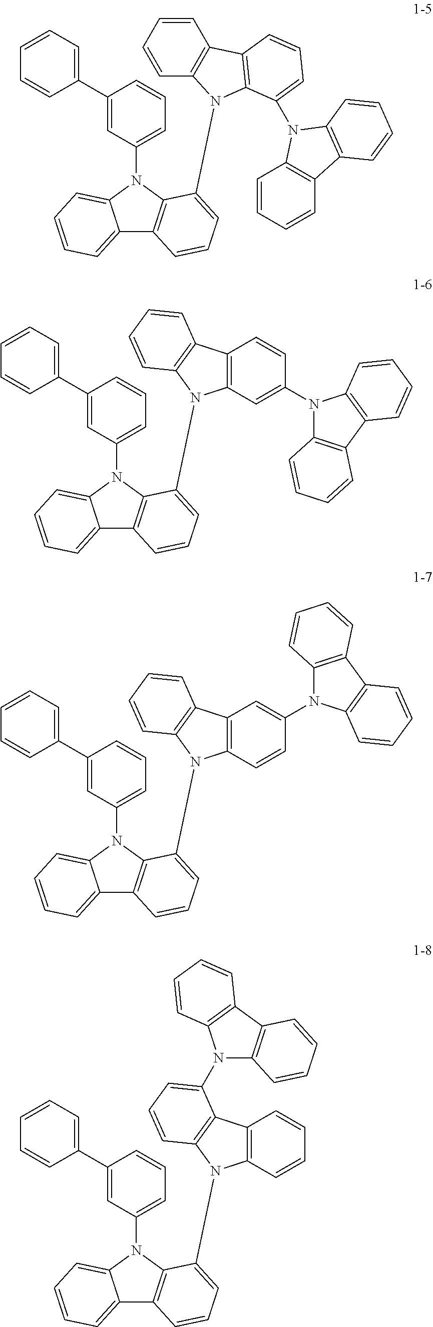 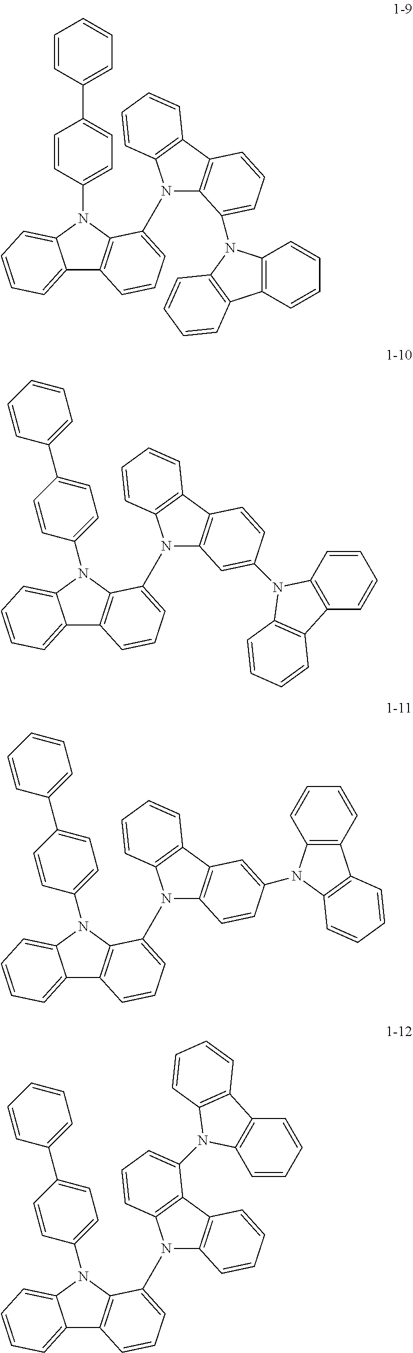 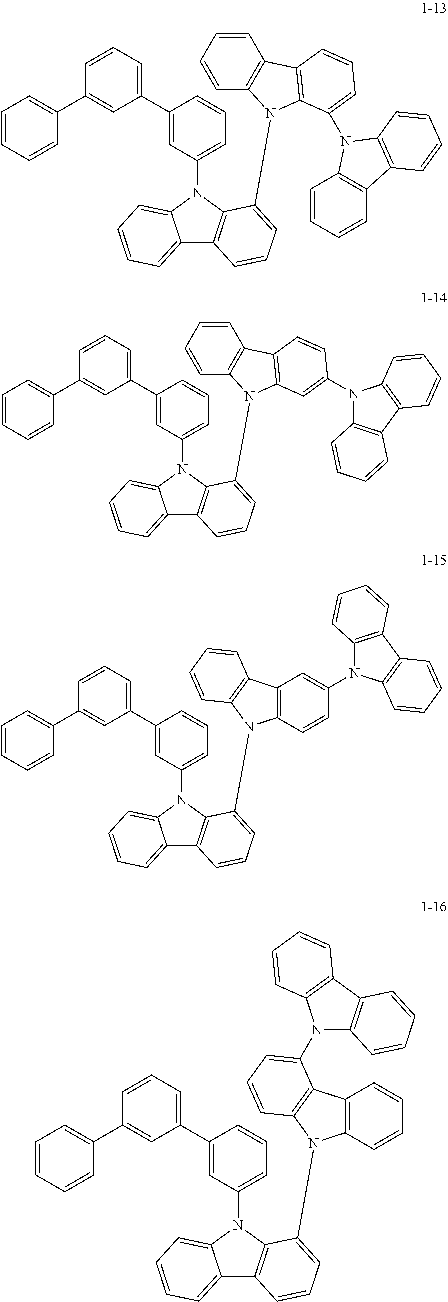 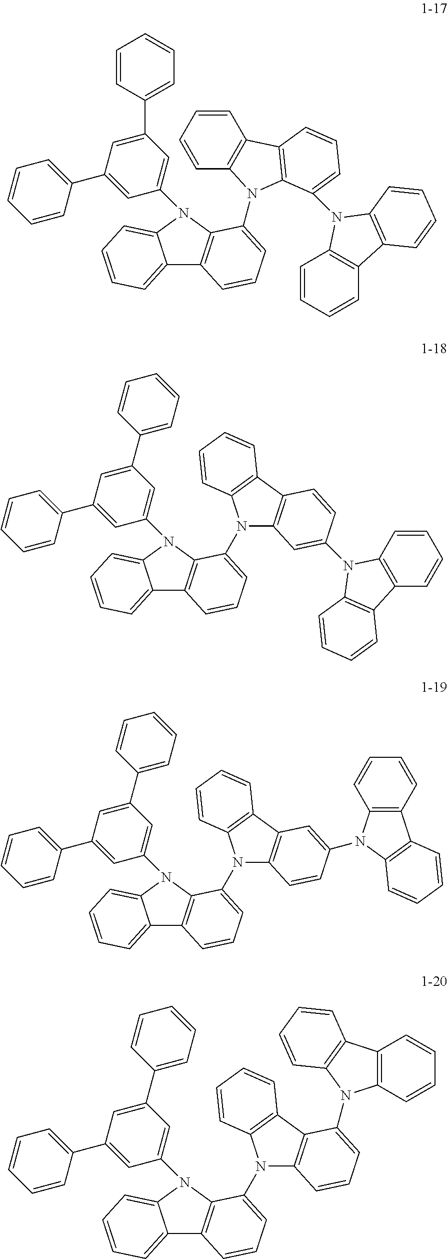 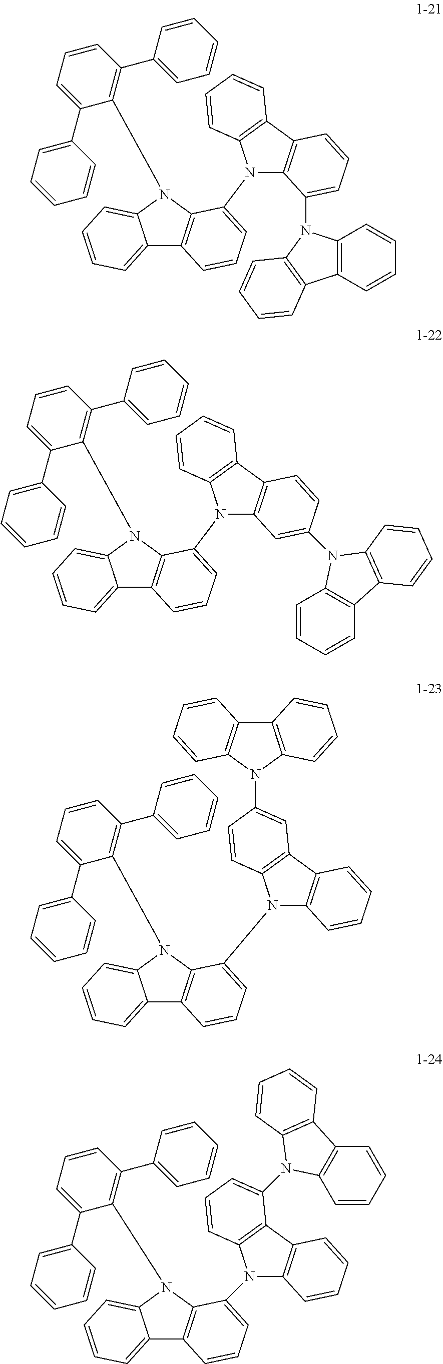 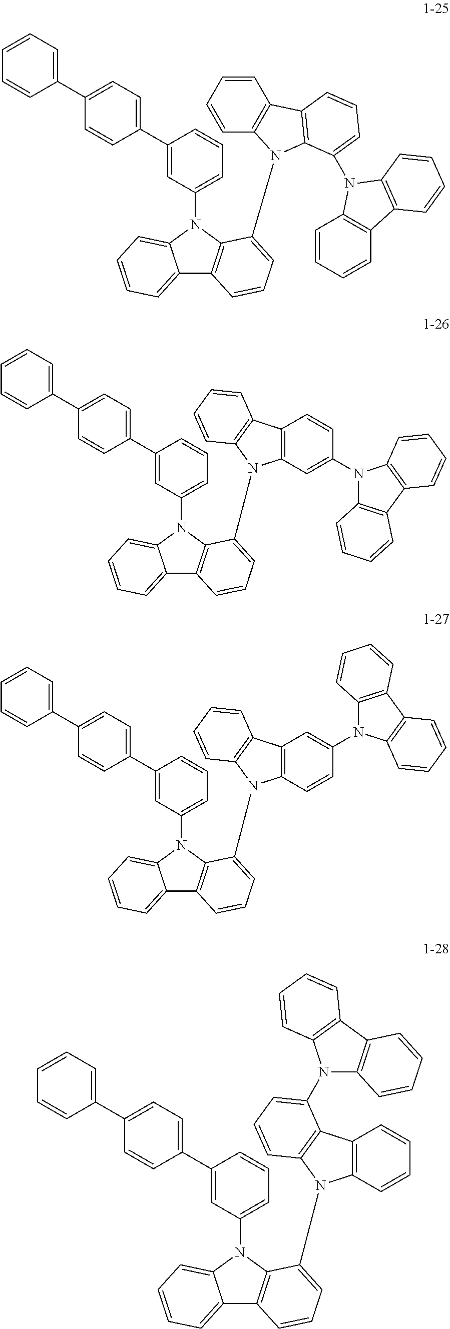 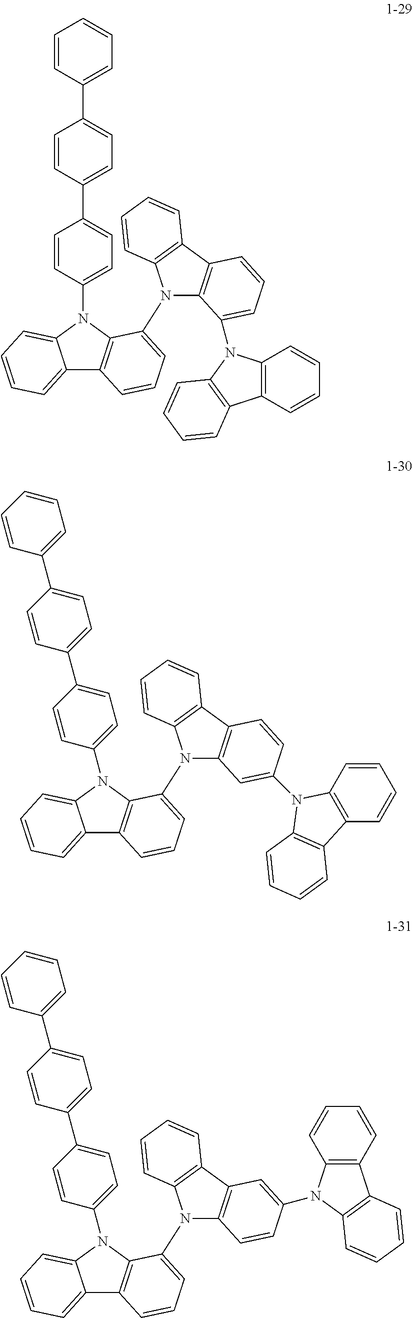 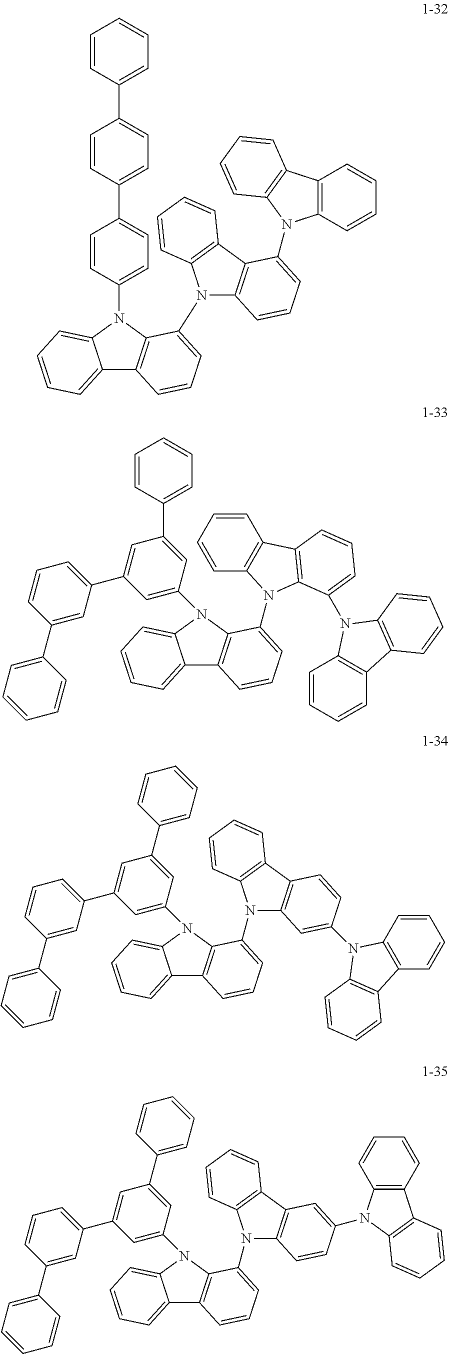 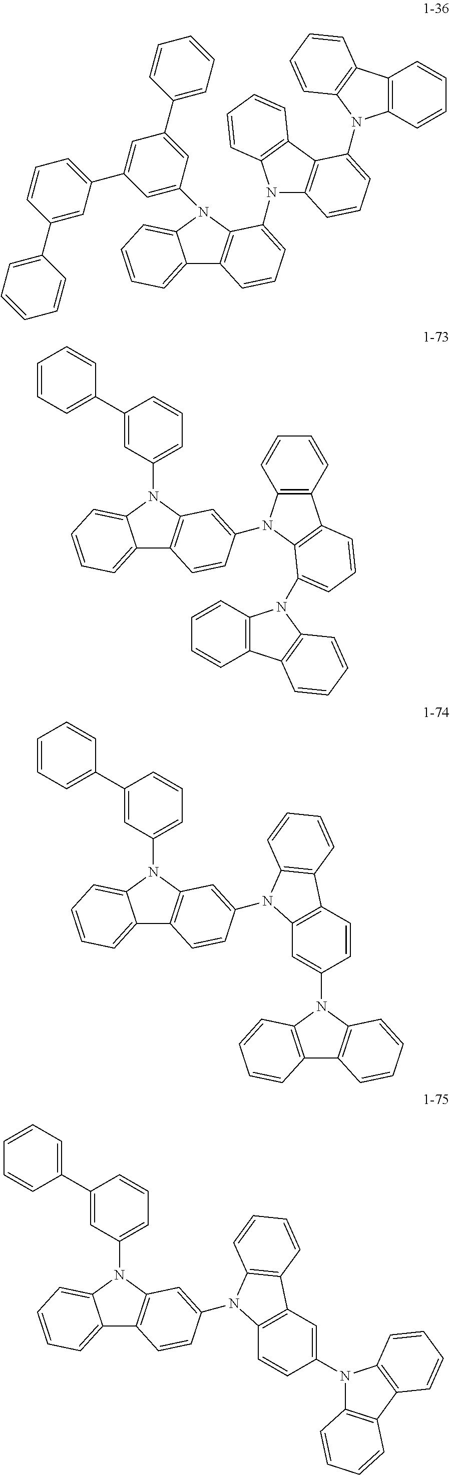 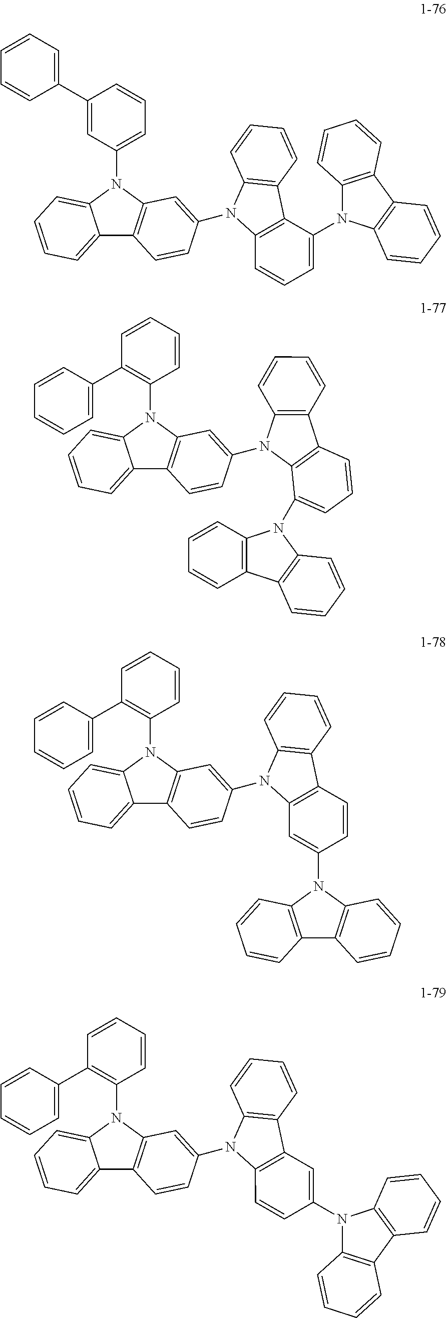 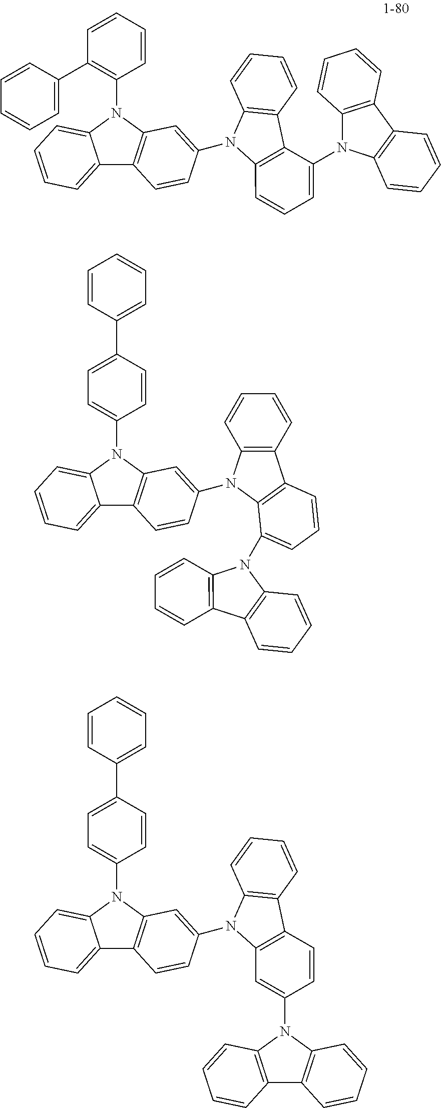 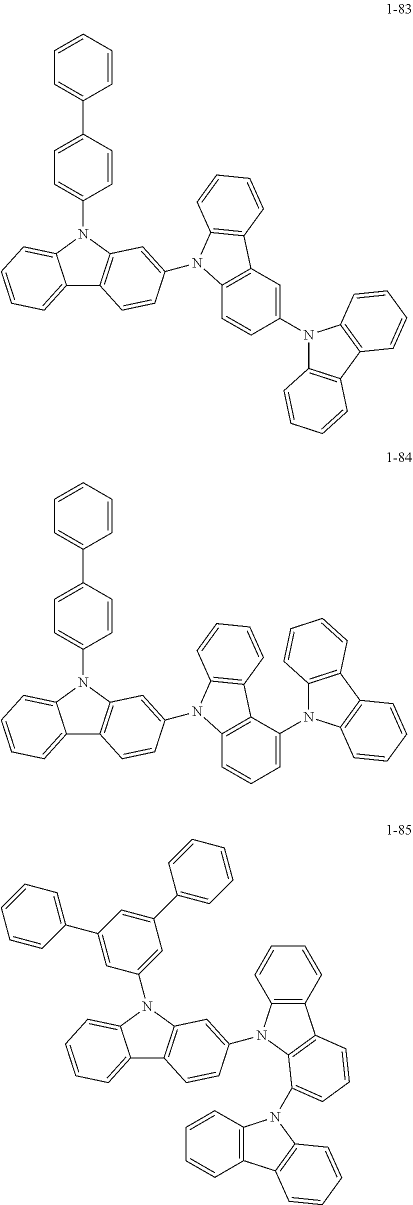 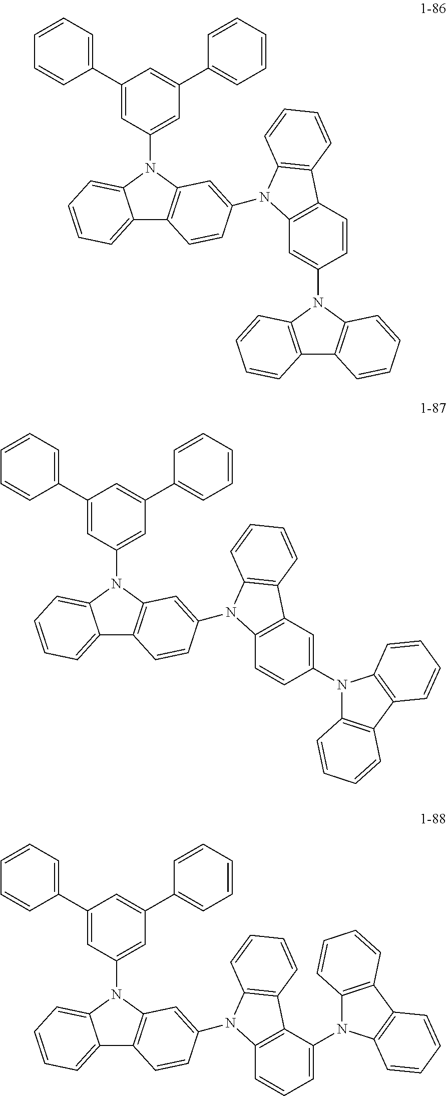 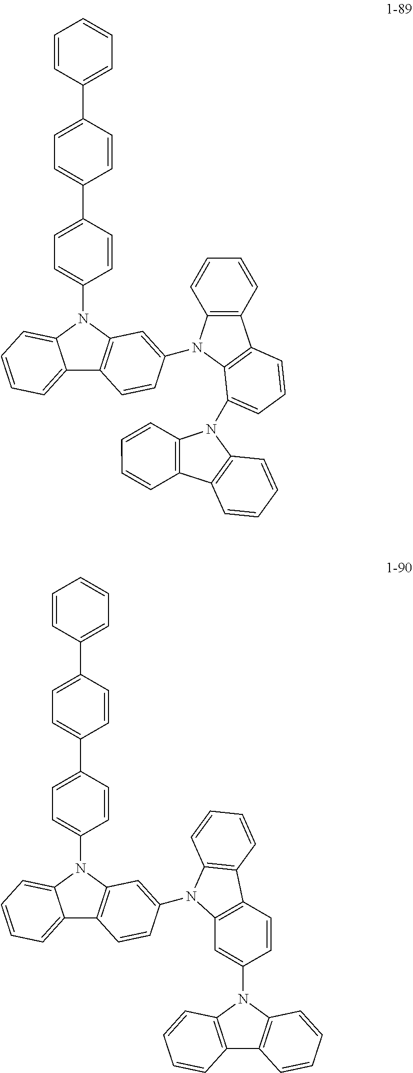 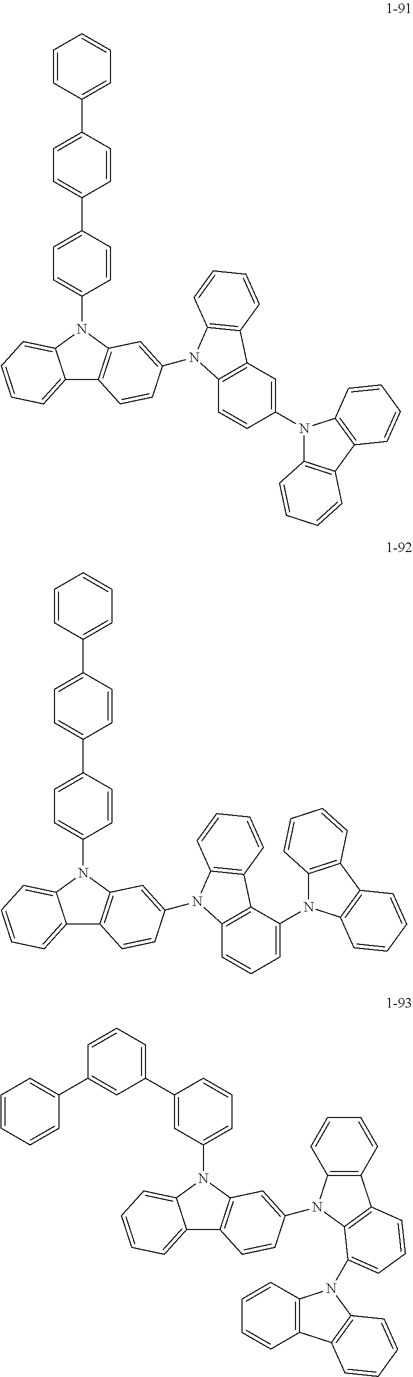 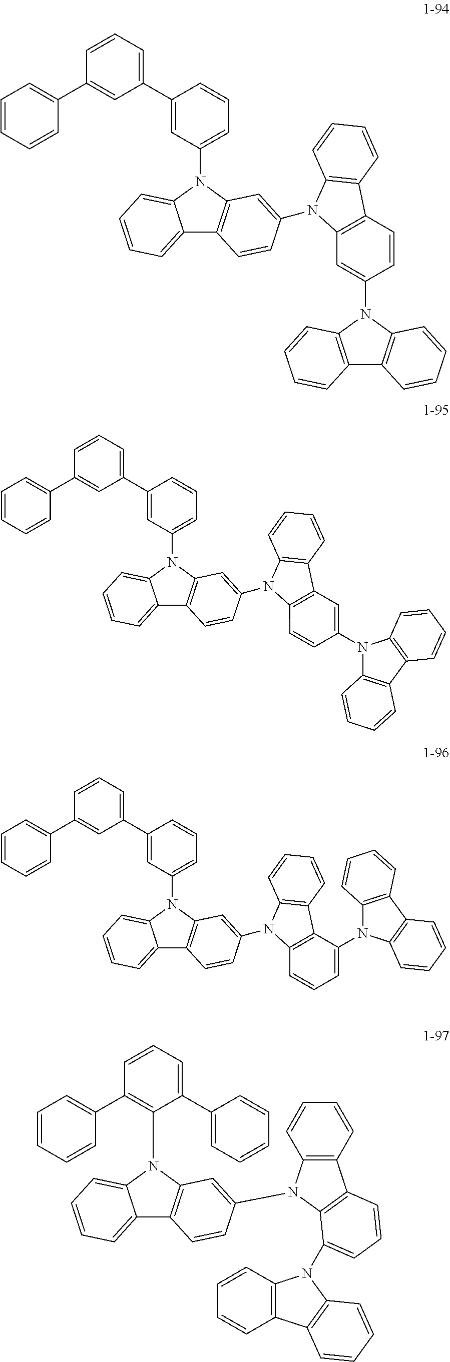 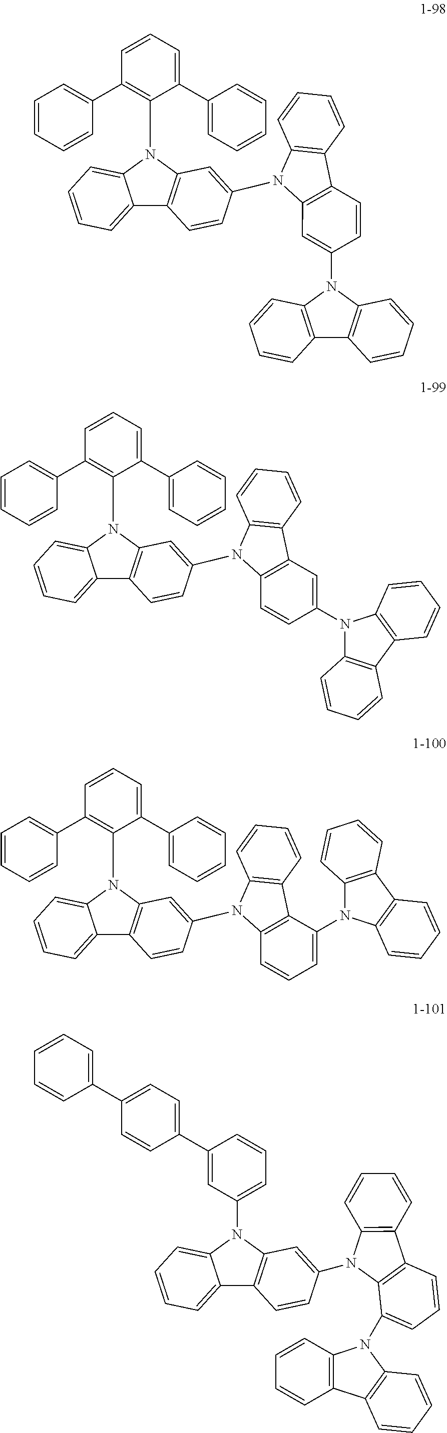 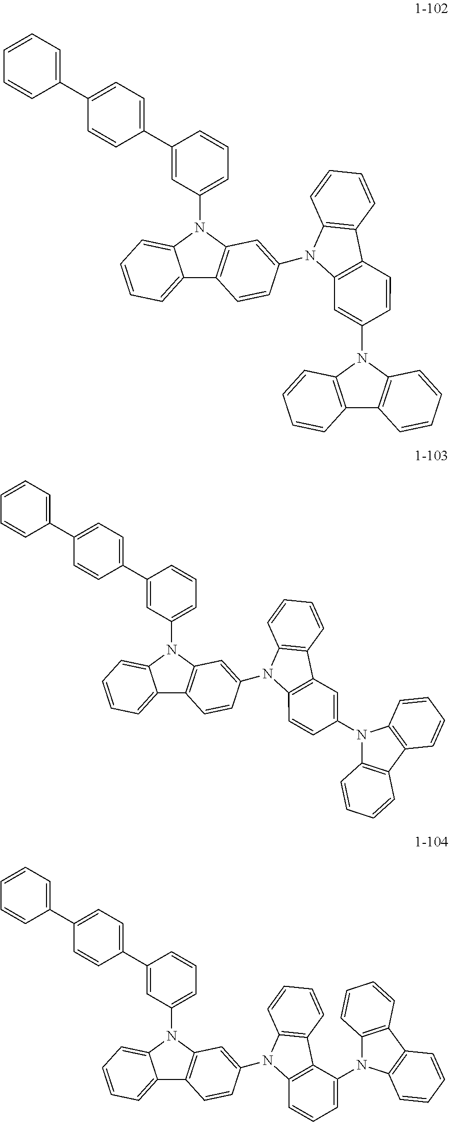 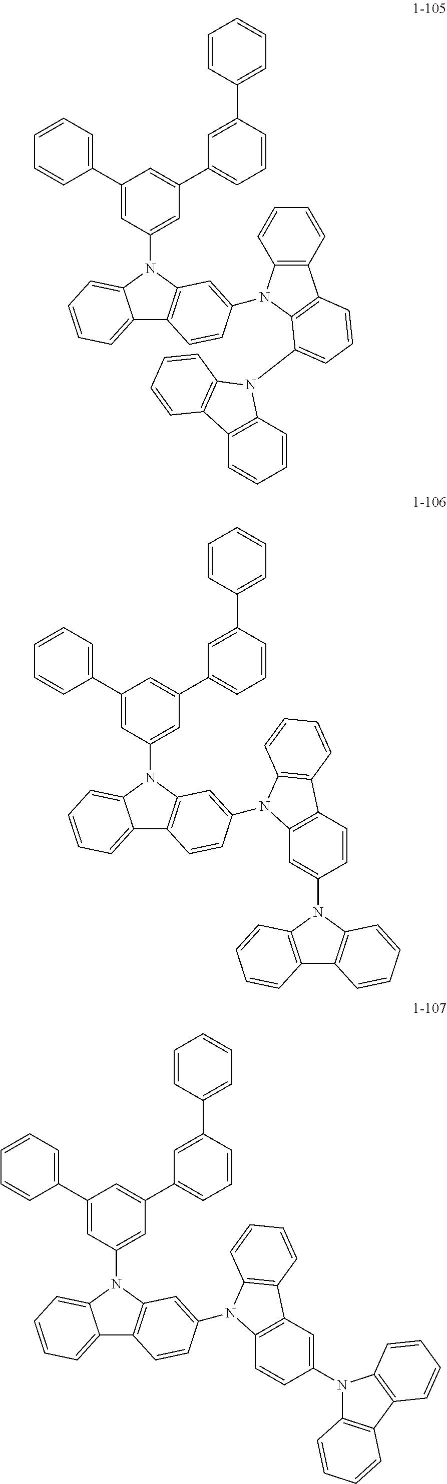 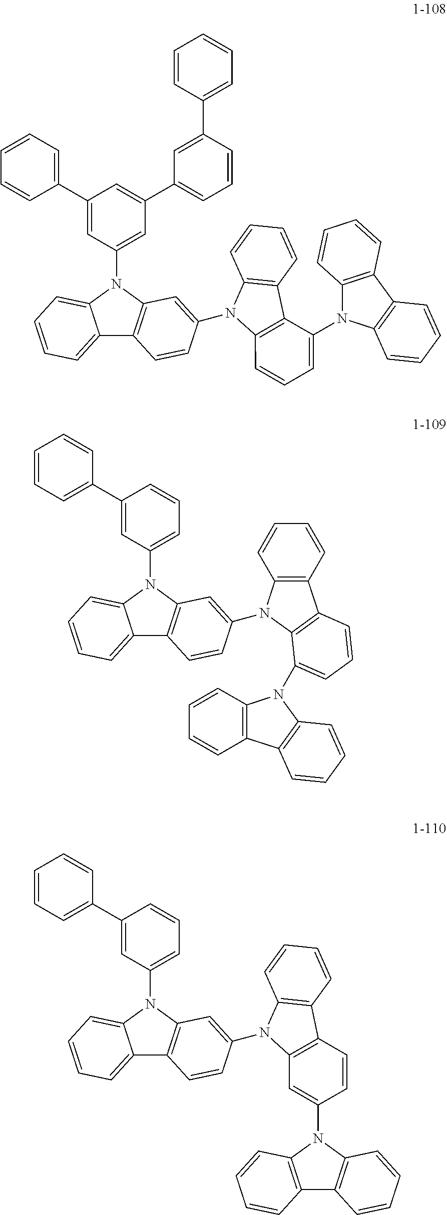 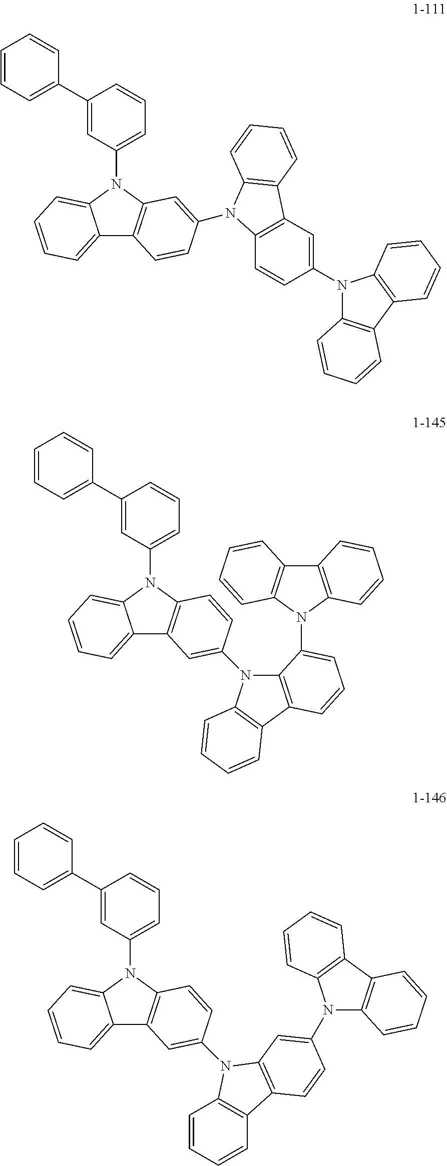 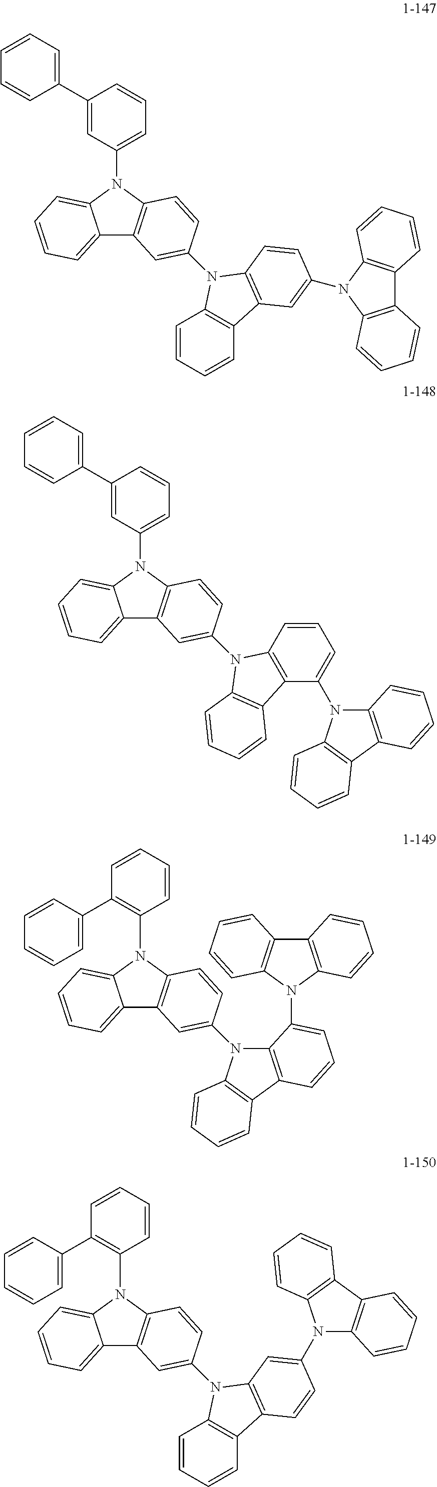 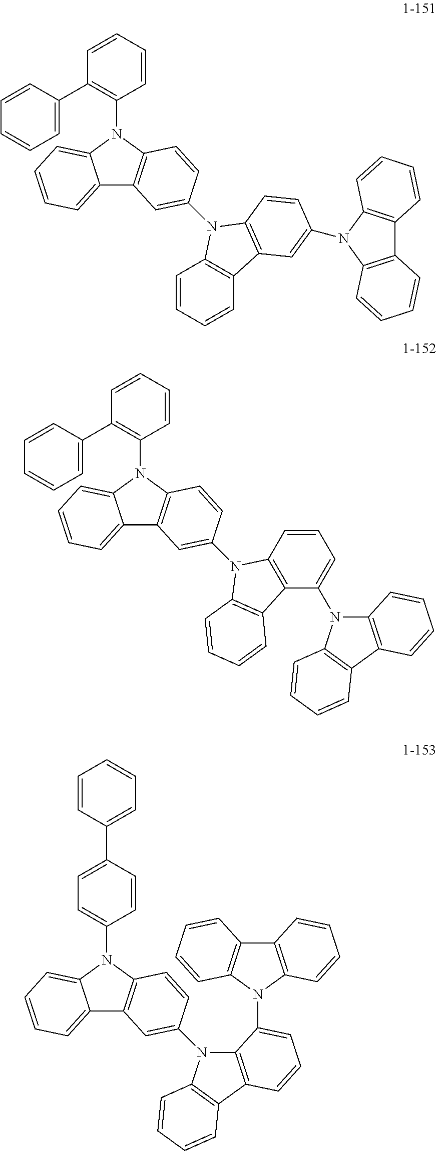 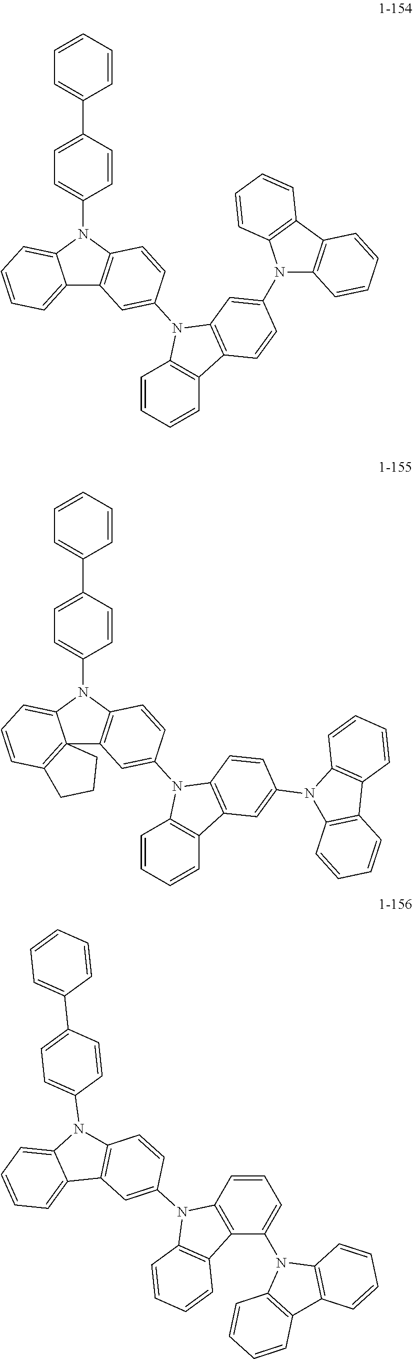 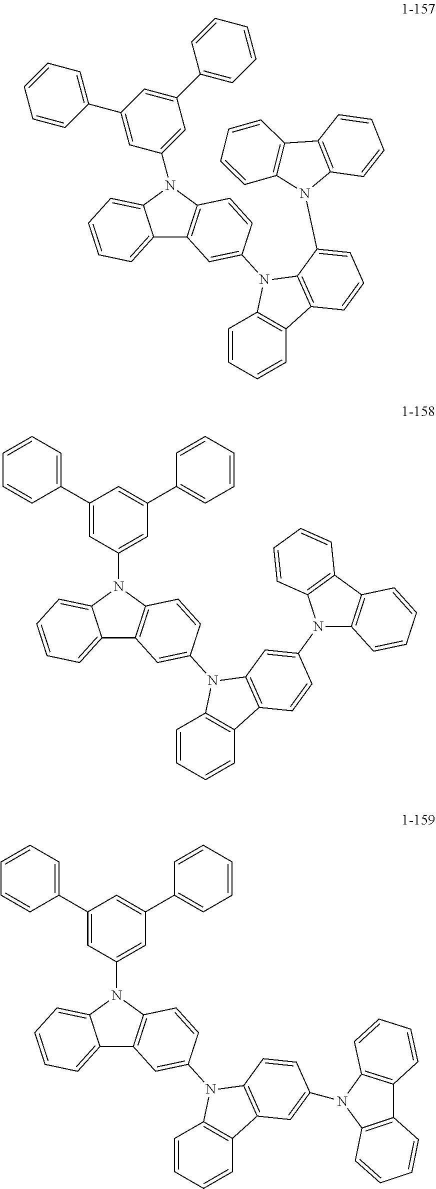 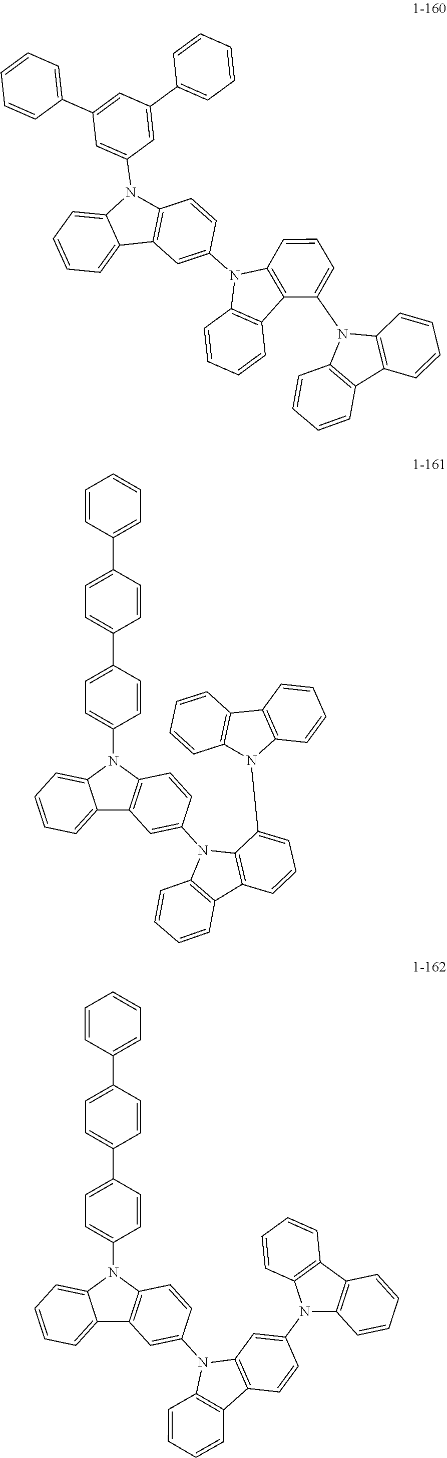 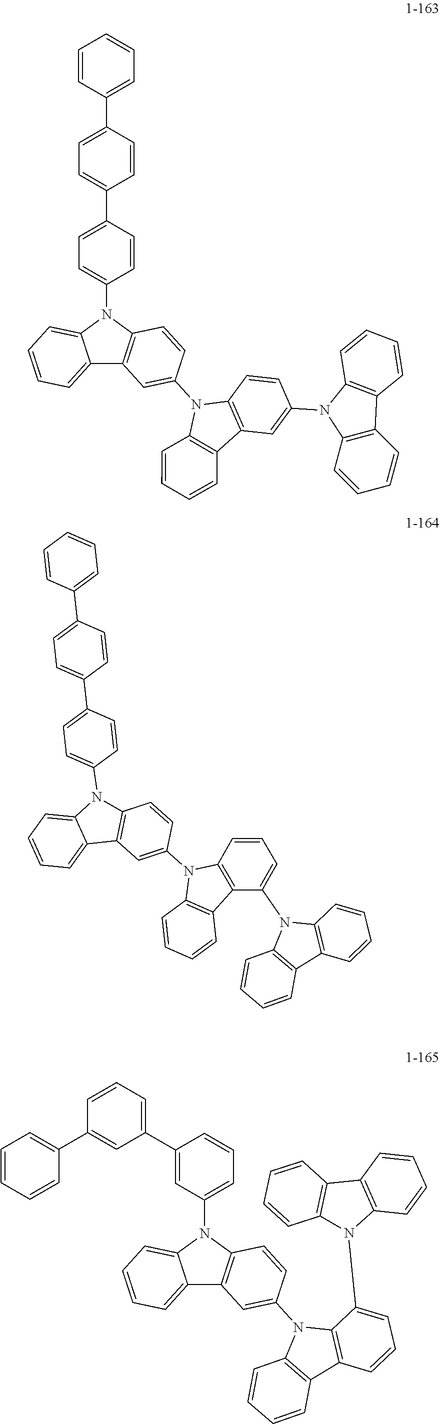 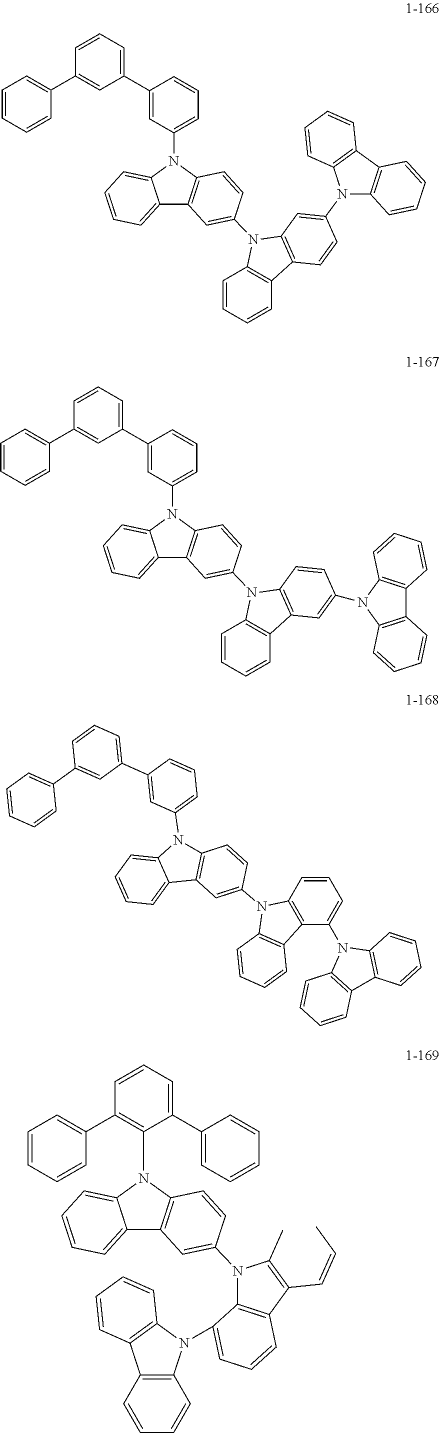 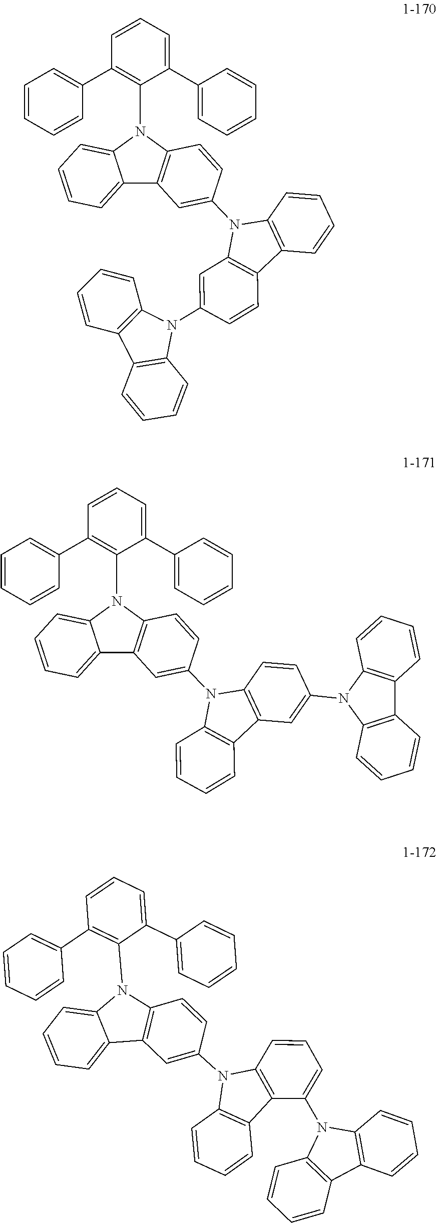 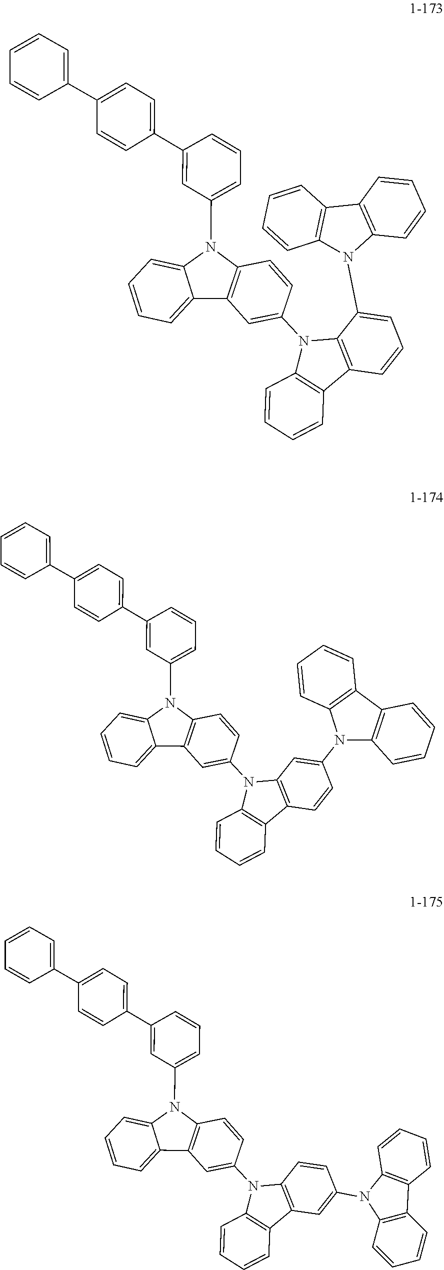 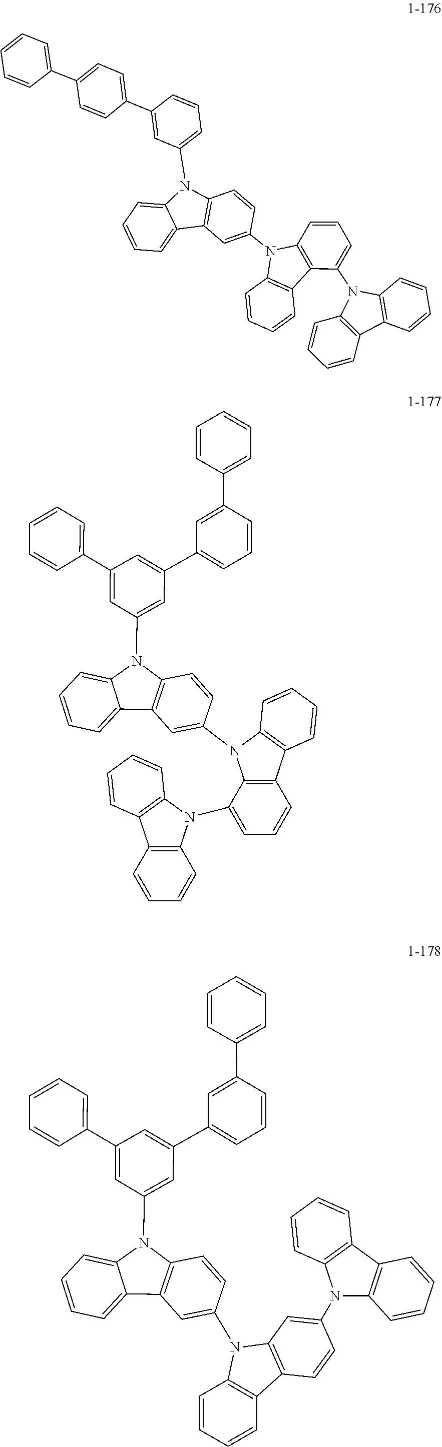 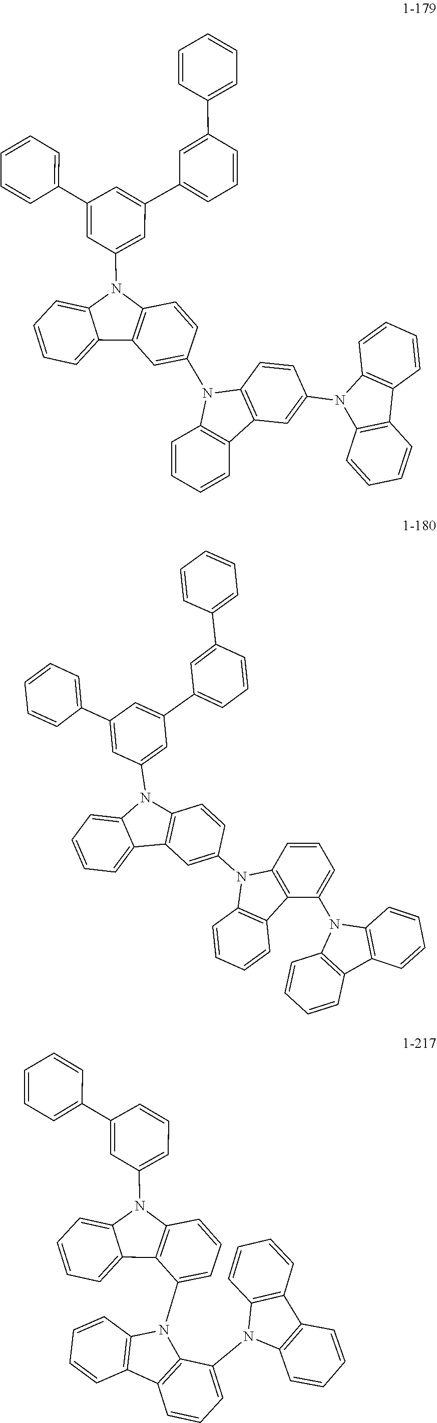 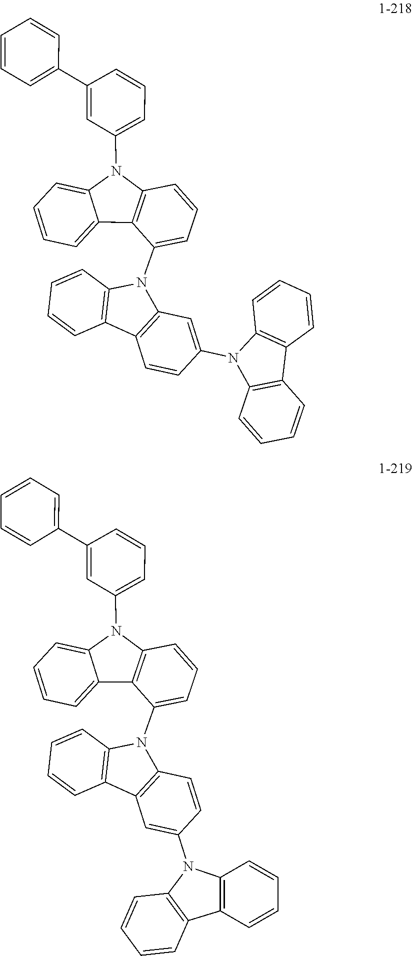 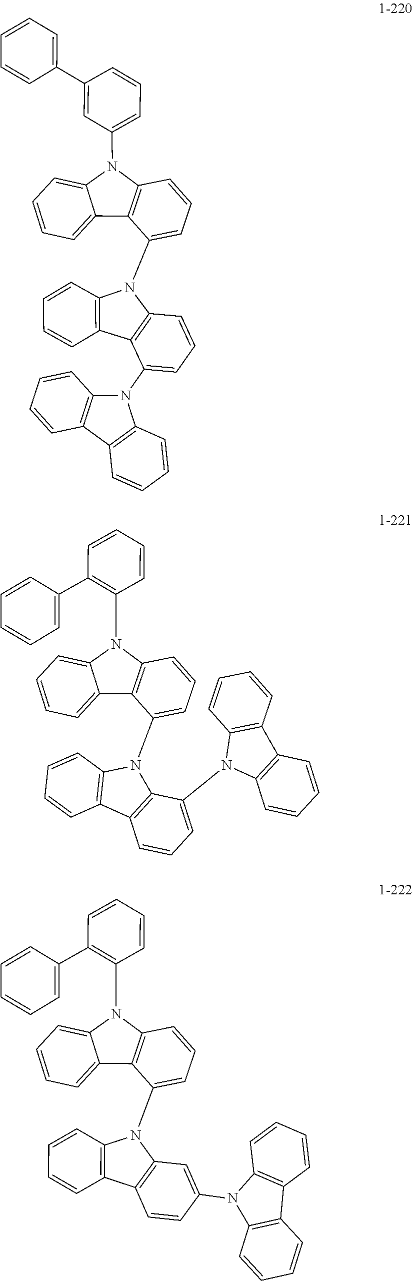 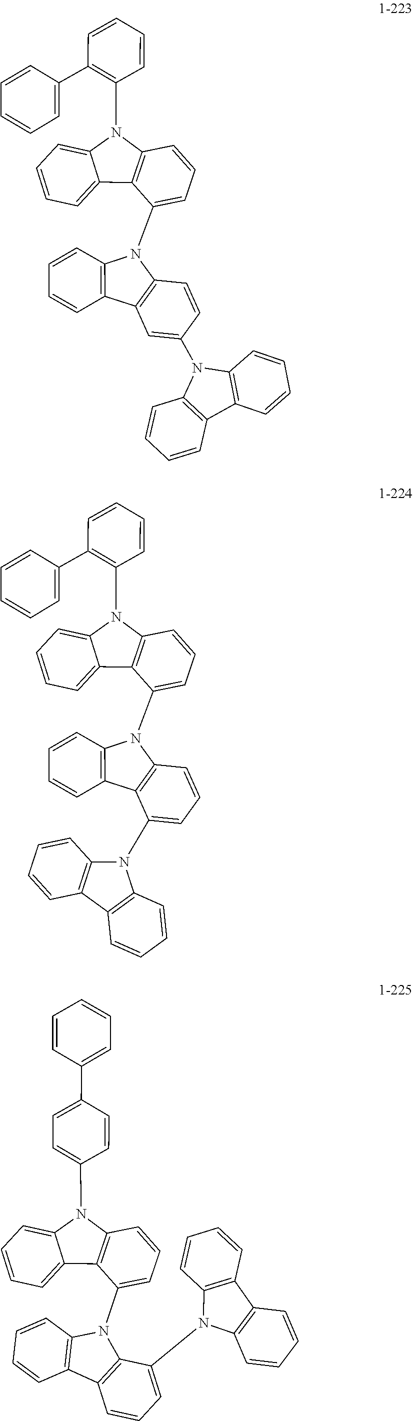 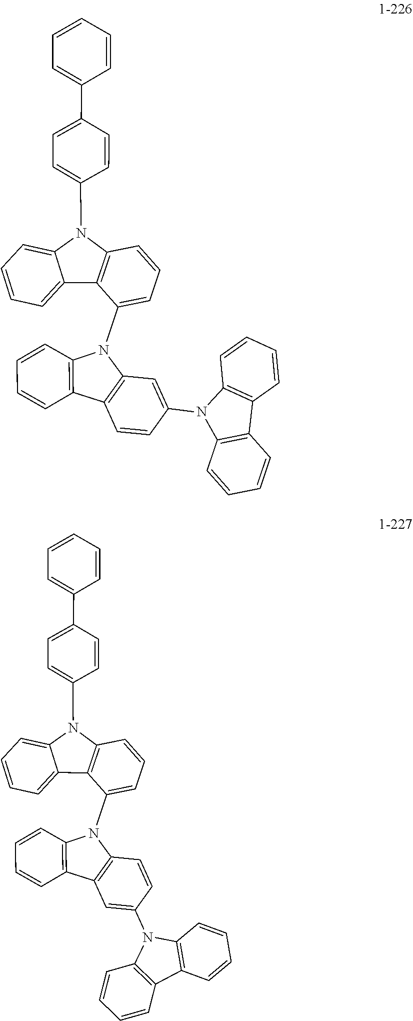 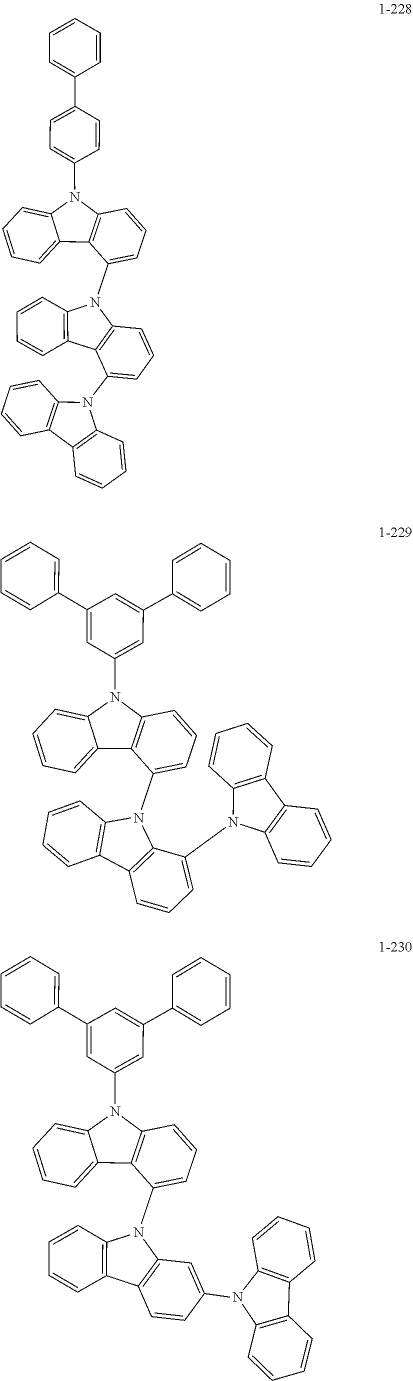 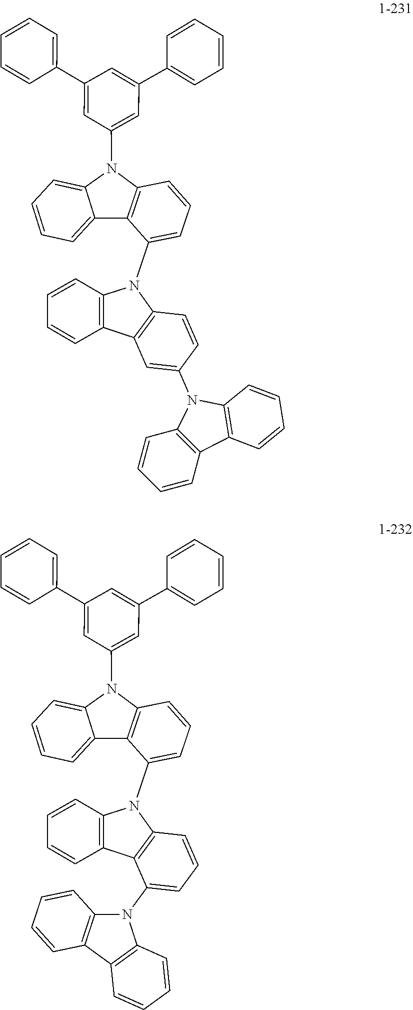 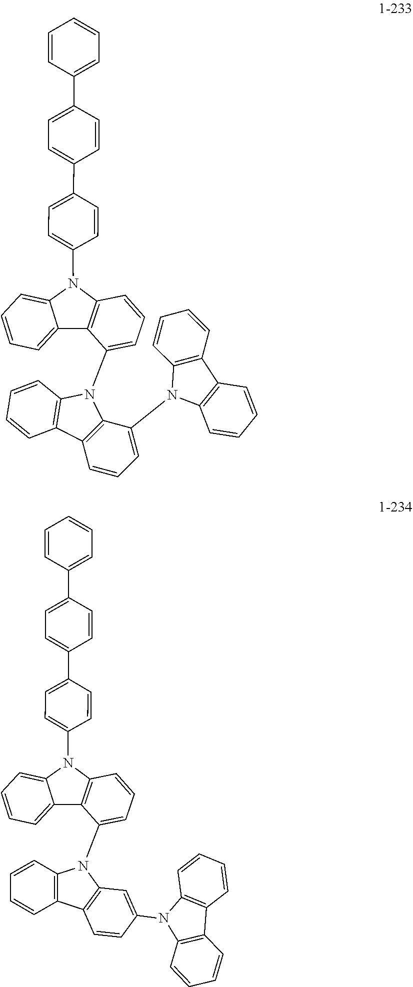 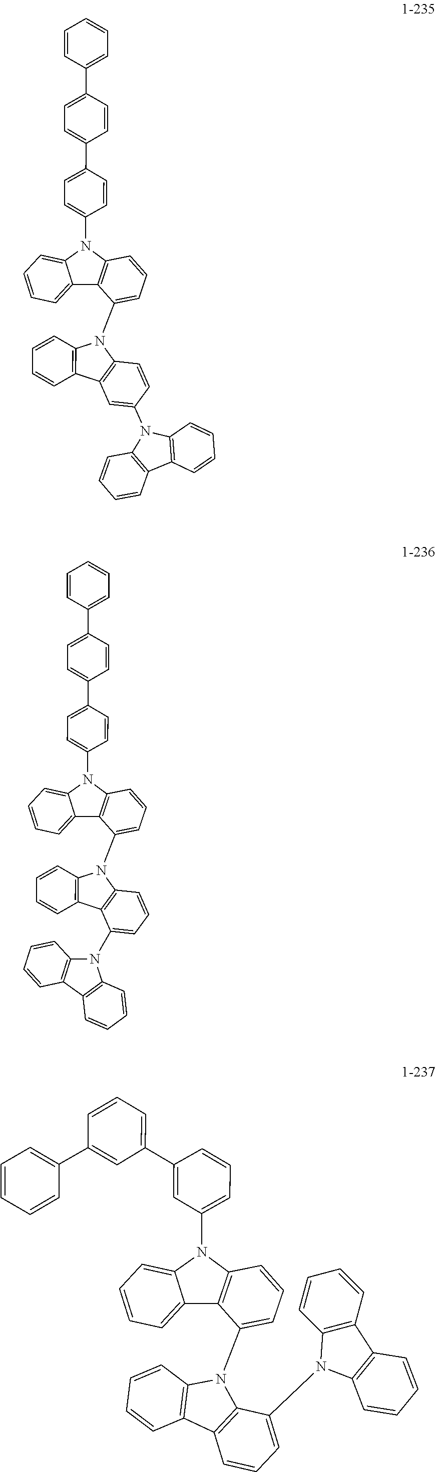 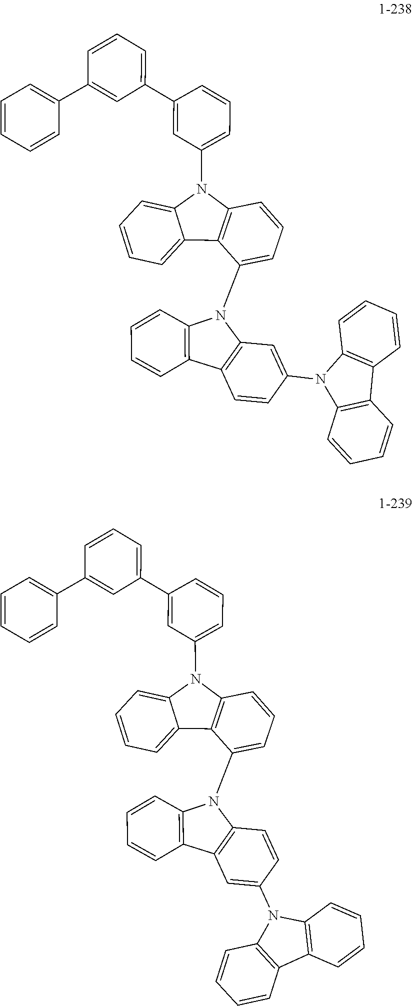 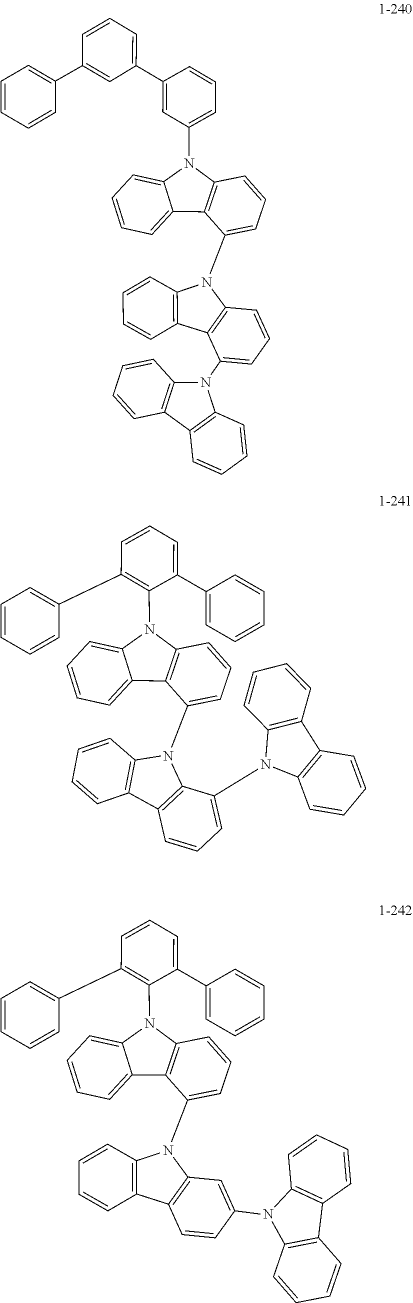 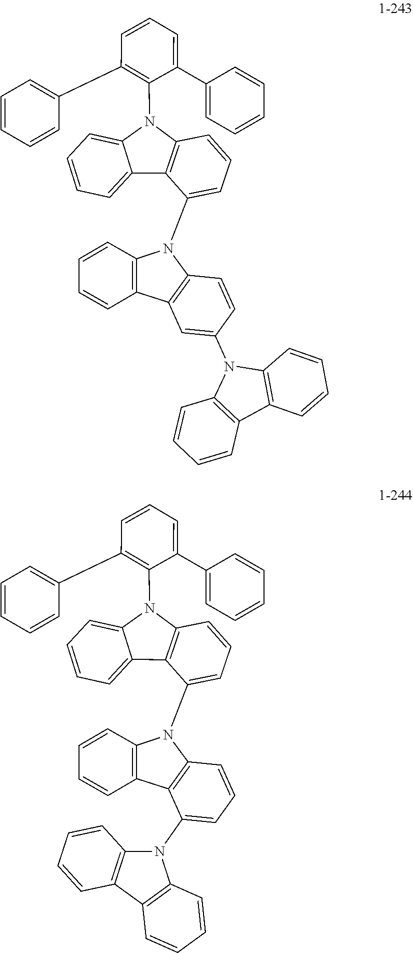 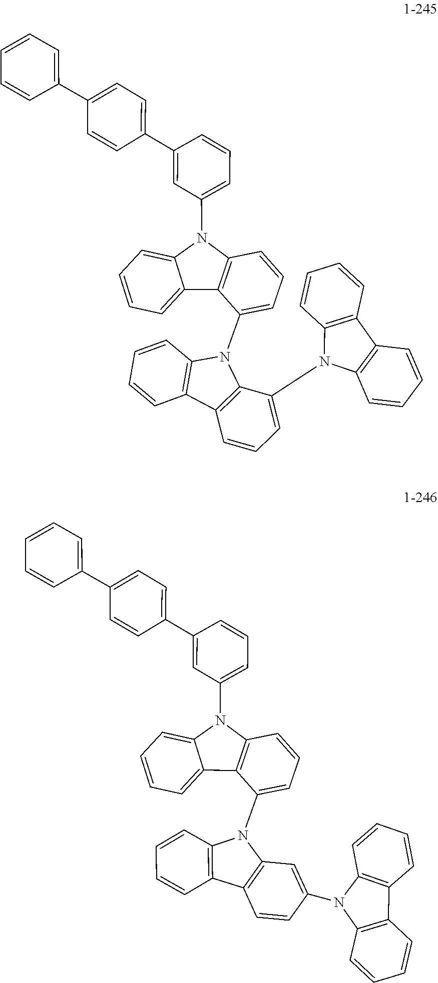 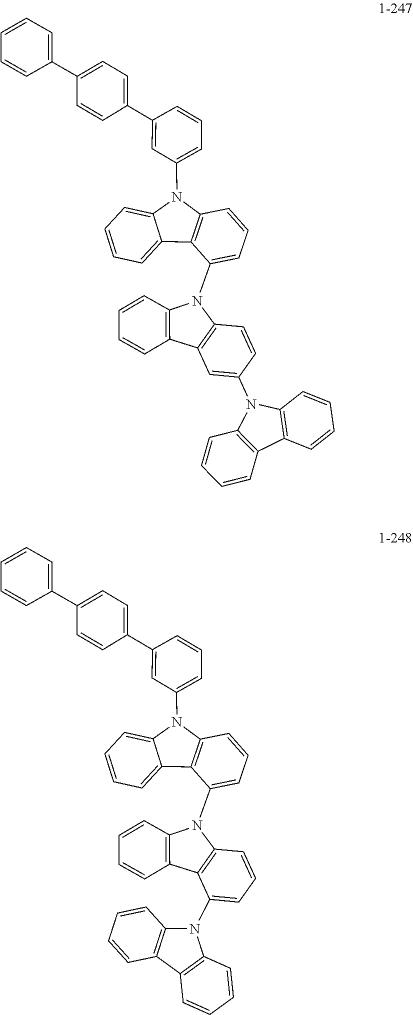 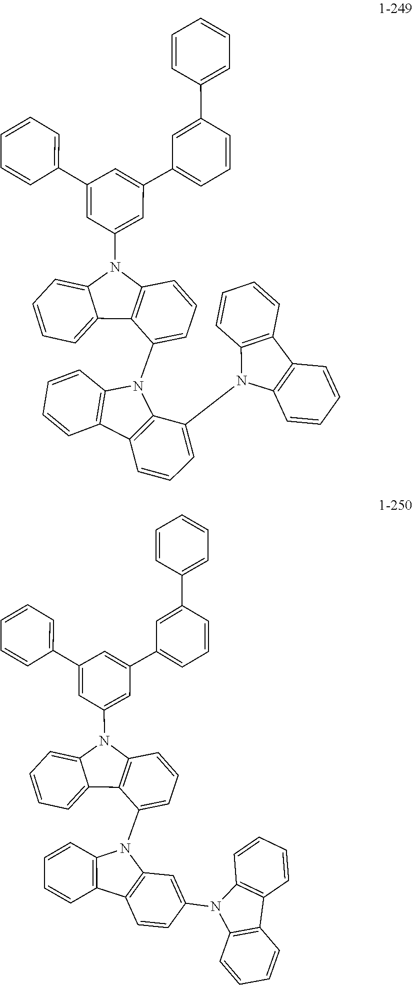 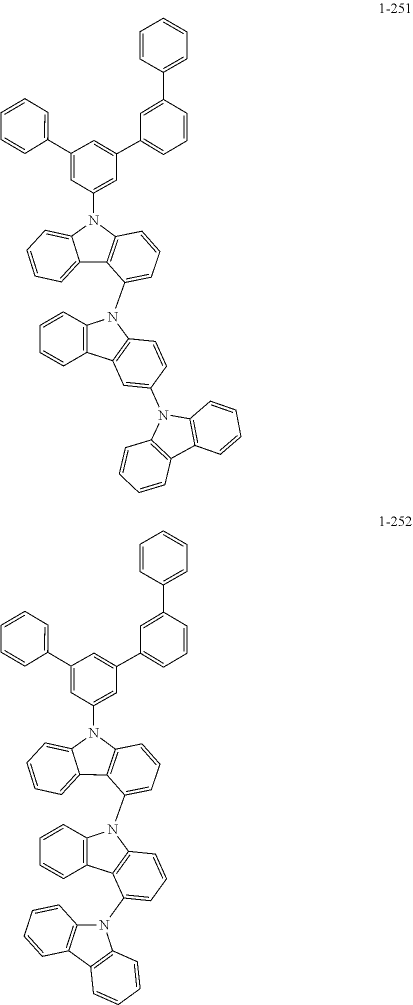
wherein, in Formulae 1-1 to 1-36, 1-73 to 1-111, 1-145 to 1-180, and 1-217 to 1-252,
at least one hydrogen is substituted with deuterium.

11. An organic light-emitting device, comprising:
a first electrode;
a second electrode; and
an organic layer arranged between the first electrode and the second electrode, wherein the organic layer comprises an emission layer,
wherein the organic layer further comprises at least one of the heterocyclic compound of claim 1.

12. The organic light-emitting device of claim 11, wherein
the organic layer further comprises a hole transport region arranged between the first electrode and the emission layer, and an electron transport region arranged between the emission layer and the second electrode,
the hole transport region comprises a hole injection layer, a hole transport layer, an electron blocking layer, an auxiliary layer, or a combination thereof, and
the electron transport region comprises a buffer layer, a hole blocking layer, an electron transport layer, an electron injection layer, or a combination thereof.

13. The organic light-emitting device of claim 11, wherein the emission layer comprises the at least one of the heterocyclic compound.

14. The organic light-emitting device of claim 12, wherein the hole transport region comprises the at least one of the heterocyclic compound.

15. The organic light-emitting device of claim 12, wherein the electron transport region comprises the at least one of the heterocyclic compound.

16. The organic light-emitting device of claim 13, wherein the emission layer further comprises a phosphorescent dopant.

17. The organic light-emitting device of claim 16, wherein the emission layer further comprises a fluorescent dopant.

18. The organic light-emitting device of claim 13, wherein the emission layer further comprises an electron-transporting host.

19. The organic light-emitting device of claim 18, wherein the electron-transporting host comprises a cyano group, a TT electron-deficient nitrogen-containing C₁-C₆₀ cyclic group, a group represented by one of the following formulae, or a combination thereof.

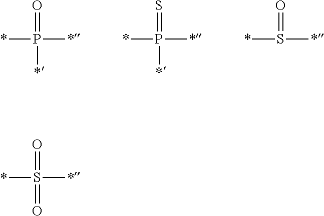

20. An electronic apparatus, comprising the organic light-emitting device of claim 12.
